(19) United States
(12) Patent Application Publication
(10) Pub. No.: US 2021/0115453 A1
(43) Pub. Date: Apr. 22, 2021

(54) TRANSPOSON SYSTEM AND METHODS OF USE

(71) Applicant: Poseida Therapeutics, Inc., San Diego, CA (US)

(72) Inventors: Devon SHEDLOCK, San Diego, CA (US); David HERMANSON, San Diego, CA (US); Eric OSTERTAG, San Diego, CA (US); Maximilian RICHTER, San Diego, CA (US); Stacey Ann CRANERT, San Diego, CA (US)

(21) Appl. No.: 16/640,807

(22) PCT Filed: Aug. 31, 2018
(86) PCT No.: PCT/US2018/049257

§ 371 Date: Feb. 21, 2020

Related U.S. Application Data
(60) Provisional application No. 62/608,546, filed on Dec. 20, 2017.
(60) Provisional application No. 62/558,286, filed on Sep. 13, 2017.
(60) Provisional application No. 62/552,861, filed on Aug. 31, 2017.

(51) Int. Cl. C12N 15/52; C12N 5/078; C12N 9/12

(52) U.S. Cl. CPC C12N 15/52; C12N 5/0634; C12N 9/1241; C12N 2501/2302; C12N 2501/2307; C12N 2501/26; C12N 2501/2321; C12N 2501/22; C12N 2501/125; C12N 2501/145; C12N 2501/2315

(57) ABSTRACT

Disclosed are methods for the ex-vivo genetic modification of an immune cell comprising delivering to the immune cell, (a) a nucleic acid or amino acid sequence comprising a sequence encoding a transposase enzyme and (b) a recombinant and non-naturally occurring DNA sequence comprising a DNA sequence encoding a transposon.

### RELATED APPLICATIONS

This application claims the benefit of provisional application U.S. Ser. No. 62/552,861, filed Aug. 31, 2017, U.S. Ser. No. 62/558,286, filed Sep. 13, 2017 and U.S. Ser. No. 62/608,546, filed Dec. 20, 2017, the contents of each of which are herein incorporated by reference in their entirety.

### INCORPORATION OF SEQUENCE LISTING

The contents of the text file named “POTH-029/001WO_SeqList.txt,” which was created on Aug. 31, 2018 and is 44,366 KB in size, are hereby incorporated by reference in their entirety.

### FIELD OF THE DISCLOSURE

The present invention is directed to compositions and methods for targeted gene modification.

### BACKGROUND

Ex vivo genetic modification of non-transformed primary human T lymphocytes using non-viral vector-based gene transfer delivery systems has been extremely difficult. As a result, most groups have generally used viral vector-based transduction such as retrovirus, including lentivirus. A number of non-viral methods have been tested and include antibody-targeted liposomes, nanoparticles, aptamer siRNA chimeras, electroporation, nucleofection, lipofection, and peptide transduction. Overall, these approaches have resulted in poor transfection efficiency, direct cell toxicity, or a lack of experimental throughput.

The use of plasmid vectors for genetic modification of human lymphocytes has been limited by low efficiency using currently available plasmid transfection systems and by the toxicity that many plasmid transfection reagents have on these cells. There is a long-felt and unmet need for a method of nonviral gene modification in immune cells.

### SUMMARY

When compared with viral transduction of immune cells, such as T lymphocytes, delivery of transgenes via DNA transposons, such as piggyBac and Sleeping Beauty, offers significant advantages in ease of use, ability to delivery much larger cargo, speed to clinic and cost of production. The piggyBac DNA transposon, in particular, offers additional advantages in giving long-term, high-level and stable expression of transgenes, and in being significantly less mutagenic than a retrovirus, being non-oncogenic and being fully reversible. Previous attempts to use DNA transposons to deliver transgenes to T cells have been unsuccessful at generating commercially viable products or manufacturing methods because the previous methods have been inefficient. For example, the poor efficiency demonstrated by previous methods of using DNA transposons to deliver transgenes to T cells has resulted in the need for prolonged expansion ex vivo. Previous unsuccessful attempts by others to solve this problem have all focused on increasing the amount of DNA transposon delivered to the immune cell, which has been a strategy that worked well for non-immune cells. This disclosure demonstrates that increasing the amount of DNA transposon makes the efficiency problem worse in immune cells by increasing DNA-mediated toxicity. To solve this problem, counterintuitively, the methods of the disclosure decrease the amount of DNA delivered to the immune cell. Using the methods of the disclosure, the data provided herein demonstrate not only that decreasing the amount of DNA transposon introduced into the cell increased viability but also that this method increased the percentage of cells that harbored a transposition event, resulting in a viable commercial process and a viable commercial product. Thus, the methods of the disclosure demonstrate success where others have failed.

The disclosure provides a nonviral method for the ex-vivo genetic modification of an immune cell or an immune cell precursor comprising delivering to the immune cell or the immune cell precursor, (a) a nucleic acid or amino acid sequence comprising a sequence encoding a transposase enzyme and (b) a recombinant and non-naturally occurring DNA sequence comprising a DNA sequence encoding a transposon. In certain embodiments, the method further comprises the step of stimulating the immune cell or the immune cell precursor with one or more cytokine(s).

In certain embodiments of the methods of the disclosure, the sequence encoding a transposase enzyme is an mRNA sequence. The mRNA sequence encoding a transposase enzyme may be produced in vitro.

In certain embodiments of the methods of the disclosure, the sequence encoding a transposase enzyme is a DNA sequence. The DNA sequence encoding a transposase enzyme may be produced in vitro. The DNA sequence may be a cDNA sequence.

In certain embodiments of the methods of the disclosure, the sequence encoding a transposase enzyme is an amino acid sequence. The amino acid sequence encoding a transposase enzyme may be produced in vitro. A protein Super piggybac transposase (SPB) may be delivered following pre-incubation with transposon DNA.

In certain embodiments of the methods of the disclosure, the delivering step comprises electroporation or nucleofection of the immune cell or the immune cell precursor.

In certain embodiments of the methods of the disclosure, the method further comprises the step of stimulating the immune cell or the immune cell precursor with one or more cytokines. In certain embodiments, the step of stimulating the immune cell or the immune cell precursor with one or more cytokine(s) occurs following the delivering step. Alternatively, or in addition, in certain embodiments, the step of stimulating the immune cell or the immune cell precursor with one or more cytokine(s) occurs prior to the delivering step. In certain embodiments, the one or more cytokine(s) comprise(s) IL-2, IL-21, IL-7 and/or IL-15.

In certain embodiments of the methods of the disclosure, the immune cell or the immune cell precursor is an autologous immune cell or immune cell precursor. The immune cell or immune cell precursor may be a human immune cell, a human immune cell precursor, an autologous immune cell, and/or an autologous immune cell precursor. The immune cell may be derived from a non-autologous source, including, but not limited to a primary cell, a cultured cell or cell line, an embryonic or adult stem cell, an induced pluripotent stem cell or a transdifferentiated cell. The immune cell may have been previously genetically modified or derived from a cell or cell line that has been genetically modified. The immune cell may be modified or may be derived from a cell or cell line that has been modified to suppress one or more apoptotic pathways. The immune cell may be modified or may be derived from a cell or cell line that has been modified to be “universally” allogenic by a majority of recipients in the context, for example, of a therapy involving an adoptive cell transfer.

In certain embodiments of the methods of the disclosure, the immune cell is an activated immune cell.

In certain embodiments of the methods of the disclosure, the immune cell is a resting immune cell.

In certain embodiments of the methods of the disclosure, the immune cell is a T-lymphocyte. In certain embodiments, the T-lymphocyte is an activated T-lymphocyte. In certain embodiments, the T-lymphocyte is a resting T-lymphocyte.

In certain embodiments of the methods of the disclosure, the immune cell is a Natural Killer (NK) cell.

In certain embodiments of the methods of the disclosure, the immune cell is a Cytokine-induced Killer (CIK) cell.

In certain embodiments of the methods of the disclosure, the immune cell is a Natural Killer T (NKT) cell.

In certain embodiments of the methods of the disclosure, the immune cell is isolated or derived from a human.

In certain embodiments of the methods of the disclosure, the immune cell precursor is a stem cell or stem-like cell capable of differentiation into an immune cell. In some embodiments, the immune cell precursor is a hematopoietic stem cell (HSC). In some embodiments, the immune cell precursor is a primitive hematopoietic stem cell. In some embodiments, the immune cell precursor is a human HSC or human primitive HSC.

In certain embodiments of the methods of the disclosure, the method further comprising the step of differentiating the immune cell precursor into an immune cell. In some embodiments, the immune cell is a T lymphocyte (T cell), a B lymphocyte (B cell), a Natural Killer (NK) cell, or a Cytokine-induced Killer (CIK) cell.

In certain embodiments of the methods of the disclosure, the immune cell is isolated or derived from a non-human mammal. In certain embodiments, the non-human mammal is a rodent, a rabbit, a cat, a dog, a pig, a horse, a cow, or a camel. In certain embodiments, the immune cell is isolated or derived from a non-human primate.

In certain embodiments of the methods of the disclosure, the mRNA sequence encoding the transposase enzyme is produced in vitro.

In certain embodiments, the transposon is a piggyBac transposon or a piggyBac-like transposon. In certain embodiments, and, in particular, those embodiments wherein the transposon is a piggyBac transposon, the transposase is a piggyBac transposase. In certain embodiments, and, in particular, those embodiments wherein the transposon is a piggyBac-like transposon, the transposase is a piggyBac-like transposase.

In certain embodiments, the piggyBac transposase comprises an amino acid sequence comprising SEQ ID NO: 14487. In certain embodiments, and, in particular, those embodiments wherein the transposon is a piggyBac transposon, the transposase is a piggyBac™ or a Super piggyBac™ (SPB) transposase. In certain embodiments, and, in particular, those embodiments wherein the transposase is a Super piggyBac™ (SPB) transposase, the sequence encoding the transposase is an mRNA sequence.

In certain embodiments of the methods of the disclosure, the transposase enzyme is a piggyBac™ (PB) transposase enzyme. The piggyBac (PB) transposase enzyme may comprise or consist of an amino acid sequence at least 75%, 80%, 85%, 90%, 95%, 99% or any percentage in between identical to:

| (SEQ ID NO: 14487) | |
|---|---|
| 1 | MGSSLDDEHI LSALLQSDDE LVGEDSDSEI SDHVSEDDVQ SDTEEAFIDE VHEVQPTSSG |
| 61 | SEILDEQNVI EQPGSSLASN RILTLPQRTI RGKNKHCWST SKSTRRSRVS ALNIVRSQRG |
| 121 | PTRMCRNIYD PLLCFKLFFT DEIISEIVKW TNAEISLKRR ESMTGATFRD TNEDEIYAFF |
| 181 | GILVMTAVRK DNHMSTDDLF DRSLSMVYVS VMSRDRFDFL IRCLRMDDKS IRPTLRENDV |
| 241 | FTPVRKIWDL FIHQCIQNYT PGAHLTIDEQ LLGFRGRCPF RMYIPNKPSK YGIKILMMCD |
| 301 | SGTKYMINGM PYLGRGTQTN GVPLGEYYVK ELSKPVHGSC RNITCDNWFT SIPLAKNLLQ |
| 361 | EPYKLTIVGT VRSNKREIPE VLKNSRSRPV GTSMFCFDGP LTLVSYKPKP AKMVYLLSSC |
| 421 | DEDASINEST GKPQMVMYYN QTKGGVDTLD QMCSVMTCSR KTNRWPMALL YGMINIACIN |
| 481 | SFIIYSHNVS SKGEKVQSRK KFMRNLYMSL TSSFMRKRLE APTLKRYLRD NISNILPNEV |
| 541 | PGTSDDSTEE PVMKKRTYCT YCPSKIRRKA NASCKKCKKV ICREHNIDMC QSCF. |

In certain embodiments of the methods of the disclosure, the transposase enzyme is a piggyBac™ (PB) transposase enzyme that comprises or consists of an amino acid sequence having an amino acid substitution at one or more of positions 30, 165, 282, or 538 of the sequence:

| (SEQ ID NO: 14487) | |
|---|---|
| 1 | MGSSLDDEHI LSALLQSDDE LVGEDSDSEI SDHVSEDDVQ SDTEEAFIDE VHEVQPTSSG |
| 61 | SEILDEQNVI EQPGSSLASN RILTLPQRTI RGKNKHCWST SKSTRRSRVS ALNIVRSQRG |
| 121 | PTRMCRNIYD PLLCFKLFFT DEIISEIVKW TNAEISLKRR ESMTGATFRD TNEDEIYAFF |
| 181 | GILVMTAVRK DNHMSTDDLF DRSLSMVYVS VMSRDRFDFL IRCLRMDDKS IRPTLRENDV |
| 241 | FTPVRKIWDL FIHQCIQNYT PGAHLTIDEQ LLGFRGRCPF RMYIPNKPSK YGIKILMMCD |
| 301 | SGTKYMINGM PYLGRGTQTN GVPLGEYYVK ELSKPVHGSC RNITCDNWFT SIPLAKNLLQ |
| 361 | EPYKLTIVGT VRSNKREIPE VLKNSRSRPV GTSMFCFDGP LTLVSYKPKP AKMVYLLSSC |
| 421 | DEDASINEST GKPQMVMYYN QTKGGVDTLD QMCSVMTCSR KTNRWPMALL YGMINIACIN |
| 481 | SFIIYSHNVS SKGEKVQSRK KFMRNLYMSL TSSFMRKRLE APTLKRYLRD NISNILPNEV |
| 541 | PGTSDDSTEE PVMKKRTYCT YCPSKIRRKA NASCKKCKKV ICREHNIDMC QSCF. |

In certain embodiments, the transposase enzyme is a piggyBac™ (PB) transposase enzyme that comprises or consists of an amino acid sequence having an amino acid substitution at two or more of positions 30, 165, 282, or 538 of the sequence of SEQ ID NO: 14487. In certain embodiments, the transposase enzyme is a piggyBac™ (PB) transposase enzyme that comprises or consists of an amino acid sequence having an amino acid substitution at three or more of positions 30, 165, 282, or 538 of the sequence of SEQ ID NO: 14487. In certain embodiments, the transposase enzyme is a piggyBac™ (PB) transposase enzyme that comprises or consists of an amino acid sequence having an amino acid substitution at each of the following positions 30, 165, 282, and 538 of the sequence of SEQ ID NO: 14487. In certain embodiments, the amino acid substitution at position 30 of the sequence of SEQ ID NO: 14487 is a substitution of a valine (V) for an isoleucine (I). In certain embodiments, the amino acid substitution at position 165 of the sequence of SEQ ID NO: 14487 is a substitution of a serine (S) for a glycine (G). In certain embodiments, the amino acid substitution at position 282 of the sequence of SEQ ID NO: 14487 is a substitution of a valine (V) for a methionine (M). In certain embodiments, the amino acid substitution at position 538 of the sequence of SEQ ID NO: 14487 is a substitution of a lysine (K) for an asparagine (N).

In certain embodiments of the methods of the disclosure, the transposase enzyme is a Super piggyBac™ (SPB) transposase enzyme. In certain embodiments, the Super piggyBac™ (SPB) transposase enzymes of the disclosure may comprise or consist of the amino acid sequence of the sequence of SEQ ID NO: 14487 wherein the amino acid substitution at position 30 is a substitution of a valine (V) for an isoleucine (I), the amino acid substitution at position 165 is a substitution of a serine (S) for a glycine (G), the amino acid substitution at position 282 is a substitution of a valine (V) for a methionine (M), and the amino acid substitution at position 538 is a substitution of a lysine (K) for an asparagine (N). In certain embodiments, the Super piggyBac™ (SPB) transposase enzyme may comprise or consist of an amino acid sequence at least 75%, 80%, 85%, 90%, 95%, 99% or any percentage in between identical to:

| (SEQ ID NO: 14484) | |
|---|---|
| 1 | MGSSLDDEHI LSALLQSDDE LVGEDSDSEV SDHVSEDDVQ SDTEEAFIDE VHEVQPTSSG |
| 61 | SEILDEQNVI EQPGSSLASN RILTLPQRTI RGKNKHCWST SKSTRRSRVS ALNIVRSQRG |
| 121 | PTRMCRNIYD PLLCFKLFFT DEIISEIVKW TNAEISLKRR ESMTSATFRD TNEDEIYAFF |
| 181 | GILVMTAVRK DNHMSTDDLF DRSLSMVYVS VMSRDRFDFL IRCLRMDDKS IRPTLRENDV |
| 241 | FTPVRKIWDL FIHQCIQNYT PGAHLTIDEQ LLGFRGRCPF RVYIPNKPSK YGIKILMMCD |
| 301 | SGTKYMINGM PYLGRGTQTN GVPLGEYYVK ELSKPVHGSC RNITCDNWFT SIPLAKNLLQ |
| 361 | EPYKLTIVGT VRSNKREIPE VLKNSRSRPV GTSMFCFDGP LTLVSYKPKP AKMVYLLSSC |
| 421 | DEDASINEST GKPQMVMYYN QTKGGVDTLD QMCSVMTCSR KTNRWPMALL YGMINIACIN |
| 481 | SFIIYSHNVS SKGEKVQSRK KFMRNLYMSL TSSFMRKRLE APTLKRYLRD NISNILPKEV |
| 541 | PGTSDDSTEE PVMKKRTYCT YCPSKIRRKA NASCKKCKKV ICREHNIDMC QSCF. |

In certain embodiments of the methods of the disclosure, including those embodiments wherein the transposase comprises the above-described mutations at positions 30, 165, 282 and/or 538, the piggyBac™ or Super piggyBac™ transposase enzyme may further comprise an amino acid substitution at one or more of positions 3, 46, 82, 103, 119, 125, 177, 180, 185, 187, 200, 207, 209, 226, 235, 240, 241, 243, 258, 296, 298, 311, 315, 319, 327, 328, 340, 421, 436, 456, 470, 486, 503, 552, 570 and 591 of the sequence of SEQ ID NO: 14487 or SEQ ID NO: 14484. In certain embodiments, including those embodiments wherein the transposase comprises the above-described mutations at positions 30, 165, 282 and/or 538, the piggyBac™ or Super piggyBac™ transposase enzyme may further comprise an amino acid substitution at one or more of positions 46, 119, 125, 177, 180, 185, 187, 200, 207, 209, 226, 235, 240, 241, 243, 296, 298, 311, 315, 319, 327, 328, 340, 421, 436, 456, 470, 485, 503, 552 and 570. In certain embodiments, the amino acid substitution at position 3 of SEQ ID NO: 14487 or SEQ ID NO: 14484 is a substitution of an asparagine (N) for a serine (S). In certain embodiments, the amino acid substitution at position 46 of SEQ ID NO: 14487 or SEQ ID NO: 14484 is a substitution of a serine (S) for an alanine (A). In certain embodiments, the amino acid substitution at position 46 of SEQ ID NO: 14487 or SEQ ID NO: 14484 is a substitution of a threonine (T) for an alanine (A). In certain embodiments, the amino acid substitution at position 82 of SEQ ID NO: 14487 or SEQ ID NO: 14484 is a substitution of a tryptophan (W) for an isoleucine (I). In certain embodiments, the amino acid substitution at position 103 of SEQ ID NO: 14487 or SEQ ID NO: 14484 is a substitution of a proline (P) for a serine (S). In certain embodiments, the amino acid substitution at position 119 of SEQ ID NO: 14487 or SEQ ID NO: 14484 is a substitution of a proline (P) for an arginine (R). In certain embodiments, the amino acid substitution at position 125 of SEQ ID NO: 14487 or SEQ ID NO: 14484 is a substitution of an alanine (A) a cysteine (C). In certain embodiments, the amino acid substitution at position 125 of SEQ ID NO: 14487 or SEQ ID NO: 14484 is a substitution of a leucine (L) for a cysteine (C). In certain embodiments, the amino acid substitution at position 177 of SEQ ID NO: 14487 or SEQ ID NO: 14484 is a substitution of a lysine (K) for a tyrosine (Y). In certain embodiments, the amino acid substitution at position 177 of SEQ ID NO: 14487 or SEQ ID NO: 14484 is a substitution of a histidine (H) for a tyrosine (Y). In certain embodiments, the amino acid substitution at position 180 of SEQ ID NO: 14487 or SEQ ID NO: 14484 is a substitution of a leucine (L) for a phenylalanine (F). In certain embodiments, the amino acid substitution at position 180 of SEQ ID NO: 14487 or SEQ ID NO: 14484 is a substitution of an isoleucine (I) for a phenylalanine (F). In certain embodiments, the amino acid substitution at position 180 of SEQ ID NO: 14487 or SEQ ID NO: 14484 is a substitution of a valine (V) for a phenylalanine (F). In certain embodiments, the amino acid substitution at position 185 of SEQ ID NO: 14487 or SEQ ID NO: 14484 is a substitution of a leucine (L) for a methionine (M). In certain embodiments, the amino acid substitution at position 187 of SEQ ID NO: 14487 or SEQ ID NO: 14484 is a substitution of a glycine (G) for an alanine (A). In certain embodiments, the amino acid substitution at position 200 of SEQ ID NO: 14487 or SEQ ID NO: 14484 is a substitution of a tryptophan (W) for a phenylalanine (F). In certain embodiments, the amino acid substitution at position 207 of SEQ ID NO: 14487 or SEQ ID NO: 14484 is a substitution of a proline (P) for a valine (V). In certain embodiments, the amino acid substitution at position 209 of SEQ ID NO: 14487 or SEQ ID NO: 14484 is a substitution of a phenylalanine (F) for a valine (V). In certain embodiments, the amino acid substitution at position 226 of SEQ ID NO: 14487 or SEQ ID NO: 14484 is a substitution of a phenylalanine (F) for a methionine (M). In certain embodiments, the amino acid substitution at position 235 of SEQ ID NO: 14487 or SEQ ID NO: 14484 is a substitution of an arginine (R) for a leucine (L). In certain embodiments, the amino acid substitution at position 240 of SEQ ID NO: 14487 or SEQ ID NO: 14484 is a substitution of a lysine (K) for a valine (V). In certain embodiments, the amino acid substitution at position 241 of SEQ ID NO: 14487 or SEQ ID NO: 14484 is a substitution of a leucine (L) for a phenylalanine (F). In certain embodiments, the amino acid substitution at position 243 of SEQ ID NO: 14487 or SEQ ID NO: 14484 is a substitution of a lysine (K) for a proline (P). In certain embodiments, the amino acid substitution at position 258 of SEQ ID NO: 14487 or SEQ ID NO: 14484 is a substitution of a serine (S) for an asparagine (N). In certain embodiments, the amino acid substitution at position 296 of SEQ ID NO: 14487 or SEQ ID NO: 14484 is a substitution of a tryptophan (W) for a leucine (L). In certain embodiments, the amino acid substitution at position 296 of SEQ ID NO: 14487 or SEQ ID NO: 14484 is a substitution of a tyrosine (Y) for a leucine (L). In certain embodiments, the amino acid substitution at position 296 of SEQ ID NO: 14487 or SEQ ID NO: 14484 is a substitution of a phenylalanine (F) for a leucine (L). In certain embodiments, the amino acid substitution at position 298 of SEQ ID NO: 14487 or SEQ ID NO: 14484 is a substitution of a leucine (L) for a methionine (M). In certain embodiments, the amino acid substitution at position 298 of SEQ ID NO: 14487 or SEQ ID NO: 14484 is a substitution of an alanine (A) for a methionine (M). In certain embodiments, the amino acid substitution at position 298 of SEQ ID NO: 14487 or SEQ ID NO: 14484 is a substitution of a valine (V) for a methionine (M). In certain embodiments, the amino acid substitution at position 311 of SEQ ID NO: 14487 or SEQ ID NO: 14484 is a substitution of an isoleucine (I) for a proline (P). In certain embodiments, the amino acid substitution at position 311 of SEQ ID NO: 14487 or SEQ ID NO: 14484 is a substitution of a valine for a proline (P). In certain embodiments, the amino acid substitution at position 315 of SEQ ID NO: 14487 or SEQ ID NO: 14484 is a substitution of a lysine (K) for an arginine (R). In certain embodiments, the amino acid substitution at position 319 of SEQ ID NO: 14487 or SEQ ID NO: 14484 is a substitution of a glycine (G) for a threonine (T). In certain embodiments, the amino acid substitution at position 327 of SEQ ID NO: 14487 or SEQ ID NO: 14484 is a substitution of an arginine (R) for a tyrosine (Y). In certain embodiments, the amino acid substitution at position 328 of SEQ ID NO: 14487 or SEQ ID NO: 14484 is a substitution of a valine (V) for a tyrosine (Y). In certain embodiments, the amino acid substitution at position 340 of SEQ ID NO: 14487 or SEQ ID NO: 14484 is a substitution of a glycine (G) for a cysteine (C). In certain embodiments, the amino acid substitution at position 340 of SEQ ID NO: 14487 or SEQ ID NO: 14484 is a substitution of a leucine (L) for a cysteine (C). In certain embodiments, the amino acid substitution at position 421 of SEQ ID NO: 14487 or SEQ ID NO: 14484 is a substitution of a histidine (H) for the aspartic acid (D). In certain embodiments, the amino acid substitution at position 436 of SEQ ID NO: 14487 or SEQ ID NO: 14484 is a substitution of an isoleucine (I) for a valine (V). In certain embodiments, the amino acid substitution at position 456 of SEQ ID NO: 14487 or SEQ ID NO: 14484 is a substitution of a tyrosine (Y) for a methionine (M). In certain embodiments, the amino acid substitution at position 470 of SEQ ID NO: 14487 or SEQ ID NO: 14484 is a substitution of a phenylalanine (F) for a leucine (L). In certain embodiments, the amino acid substitution at position 485 of SEQ ID NO: 14487 or SEQ ID NO: 14484 is a substitution of a lysine (K) for a serine (S). In certain embodiments, the amino acid substitution at position 503 of SEQ ID NO: 14487 or SEQ ID NO: 14484 is a substitution of a leucine (L) for a methionine (M). In certain embodiments, the amino acid substitution at position 503 of SEQ ID NO: 14487 or SEQ ID NO: 14484 is a substitution of an isoleucine (I) for a methionine (M). In certain embodiments, the amino acid substitution at position 552 of SEQ ID NO: 14487 or SEQ ID NO: 14484 is a substitution of a lysine (K) for a valine (V). In certain embodiments, the amino acid substitution at position 570 of SEQ ID NO: 14487 or SEQ ID NO: 14484 is a substitution of a threonine (T) for an alanine (A). In certain embodiments, the amino acid substitution at position 591 of SEQ ID NO: 14487 or SEQ ID NO: 14484 is a substitution of a proline (P) for a glutamine (Q). In certain embodiments, the amino acid substitution at position 591 of SEQ ID NO: 14487 or SEQ ID NO: 14484 is a substitution of an arginine (R) for a glutamine (Q).

In certain embodiments of the methods of the disclosure, including those embodiments wherein the transposase comprises the above-described mutations at positions 30, 165, 282 and/or 538, the piggyBac™ transposase enzyme may comprise or the Super piggyBac™ transposase enzyme may further comprise an amino acid substitution at one or more of positions 103, 194, 372, 375, 450, 509 and 570 of the sequence of SEQ ID NO: 14487 or SEQ ID NO: 14484. In certain embodiments of the methods of the disclosure, including those embodiments wherein the transposase comprises the above-described mutations at positions 30, 165, 282 and/or 538, the piggyBac™ transposase enzyme may comprise or the Super piggyBac™ transposase enzyme may further comprise an amino acid substitution at two, three, four, five, six or more of positions 103, 194, 372, 375, 450, 509 and 570 of the sequence of SEQ ID NO: 14487 or SEQ ID NO: 14484. In certain embodiments, including those embodiments wherein the transposase comprises the above-described mutations at positions 30, 165, 282 and/or 538, the piggyBac™ transposase enzyme may comprise or the Super piggyBac™ transposase enzyme may further comprise an amino acid substitution at positions 103, 194, 372, 375, 450, 509 and 570 of the sequence of SEQ ID NO: 14487 or SEQ ID NO: 14484. In certain embodiments, the amino acid substitution at position 103 of SEQ ID NO: 14487 or SEQ ID NO: 14484 is a substitution of a proline (P) for a serine (S). In certain embodiments, the amino acid substitution at position 194 of SEQ ID NO: 14487 or SEQ ID NO: 14484 is a substitution of a valine (V) for a methionine (M). In certain embodiments, the amino acid substitution at position 372 of SEQ ID NO: 14487 or SEQ ID NO: 14484 is a substitution of an alanine (A) for an arginine (R). In certain embodiments, the amino acid substitution at position 375 of SEQ ID NO: 14487 or SEQ ID NO: 14484 is a substitution of an alanine (A) for a lysine (K). In certain embodiments, the amino acid substitution at position 450 of SEQ ID NO: 14487 or SEQ ID NO: 14484 is a substitution of an asparagine (N) for an aspartic acid (D). In certain embodiments, the amino acid substitution at position 509 of SEQ ID NO: 14487 or SEQ ID NO: 14484 is a substitution of a glycine (G) for a serine (S). In certain embodiments, the amino acid substitution at position 570 of SEQ ID NO: 14487 or SEQ ID NO: 14484 is a substitution of a serine (S) for an asparagine (N). In certain embodiments, the piggyBac™ transposase enzyme may comprise a substitution of a valine (V) for a methionine (M) at position 194 of SEQ ID NO: 14487. In certain embodiments, including those embodiments wherein the piggyBac™ transposase enzyme may comprise a substitution of a valine (V) for a methionine (M) at position 194 of SEQ ID NO: 14487, the piggyBac™ transposase enzyme may further comprise an amino acid substitution at positions 372, 375 and 450 of the sequence of SEQ ID NO: 14487 or SEQ ID NO: 14484. In certain embodiments, the piggyBac™ transposase enzyme may comprise a substitution of a valine (V) for a methionine (M) at position 194 of SEQ ID NO: 14487, a substitution of an alanine (A) for an arginine (R) at position 372 of SEQ ID NO: 14487, and a substitution of an alanine (A) for a lysine (K) at position 375 of SEQ ID NO: 14487. In certain embodiments, the piggyBac™ transposase enzyme may comprise a substitution of a valine (V) for a methionine (M) at position 194 of SEQ ID NO: 14487, a substitution of an alanine (A) for an arginine (R) at position 372 of SEQ ID NO: 14487, a substitution of an alanine (A) for a lysine (K) at position 375 of SEQ ID NO: 14487 and a substitution of an asparagine (N) for an aspartic acid (D) at position 450 of SEQ ID NO: 14487.

In certain embodiments of the methods of the disclosure, the transposase enzyme is a Super piggyBac™ (SPB) transposase enzyme. The Super piggyBac (PB) transposase enzyme may comprise or consist of an amino acid sequence at least 75% identical to:

(SEQ ID NO: 14484)
MGSSLDDEHILSALLQSDDELVGEDSDSEVSDHVSEDDVQSDTEEAFI
DEVHEVQPTSSGSEILDEQNVIEQPGSSLASNRILTLPQRTIRGKNKH
CWSTSKSTRRSRVSALNIVRSQRGPTRMCRNIYDPLLCFKLFFTDEII
SEIVKWTNAEISLKRRESMTSATFRDTNEDEIYAFFGILVMTAVRKDN
HMSTDDLFDRSLSMVYVSVMSRDRFDFLIRCLRMDDKSIRPTLRENDV
FTPVRKIWDLFIHQCIQNYTPGAHLTIDEQLLGFRGRCPFRVYIPNKP
SKYGIKILMMCDSGTKYMINGMPYLGRGTQTNGVPLGEYYVKELSKPV
HGSCRNITCDNWFTSIPLAKNLLQEPYKLTIVGTVRSNKREIPEVLKN
SRSRPVGTSMFCFDGPLTLVSYKPKPAKMVYLLSSCDEDASINESTGK
PQMVMYYNQTKGGVDTLDQMCSVMTCSRKTNRWPMALLYGMINIACIN
SFIIYSHNVSSKGEKVQSRKKFMRNLYMSLTSSFMRKRLEAPTLKRYL
RDNISNILPKEVPGTSDDSTEEPVMKKRTYCTYCPSKIRRKANASCKK
CKKVICREHNIDMCQSCF.

In certain embodiments of the methods of the disclosure, the transposon is a Sleeping Beauty transposon. In certain embodiments of the methods of the disclosure, the transposase enzyme is a Sleeping Beauty transposase enzyme (see, for example, U.S. Pat. No. 9,228,180, the contents of which are incorporated herein in their entirety). In certain embodiments, the Sleeping Beauty transposase is a hyperactive Sleeping Beauty (SB100X) transposase. In certain embodiments, the Sleeping Beauty transposase enzyme comprises an amino acid sequence at least 75%, 80%, 85%, 90%, 95%, 99% or any percentage in between identical to:

| (SEQ ID NO: 14485) | |
|---|---|
| 1 | MGKSKEISQD LRKKIVDLHK SGSSLGAISK RLKVPRSSVQ TIVRKYKHHG TTQPSYRSGR |
| 61 | RRVLSPRDER TLVRKVQINP RTTAKDLVKM LEETGTKVSI STVKRVLYRH NLKGRSARKK |
| 121 | PLLQNRHKKA RLRFATAHGD KDRTFWRNVL WSDETKIELF GHNDHRYVWR KKGEACKPKN |
| 181 | TIPTVKHGGG SIMLWGCFAA GGTGALHKID GIMRKENYVD ILKQHLKTSV RKLKLGRKWV |
| 241 | FQMDNDPKHT SKVVAKWLKD NKVKVLEWPS QSPDLNPIEN LWAELKKRVR ARRPTNLTQL |
| 301 | HQLCQEEWAK IHPTYCGKLV EGYPKRLTQV KQFKGNATKY. |

In certain embodiments, including those wherein the Sleeping Beauty transposase is a hyperactive Sleeping Beauty (SB100X) transposase, the Sleeping Beauty transposase enzyme comprises an amino acid sequence at least at least 75%, 80%, 85%, 90%, 95%, 99% or any percentage in between identical to:

| (SEQ ID NO: 14486) | |
|---|---|
| 1 | MGKSKEISQD LRKRIVDLHK SGSSLGAISK RLAVPRSSVQ TIVRKYKHHG TTQPSYRSGR |
| 61 | RRVLSPRDER TLVRKVQINP RTTAKDLVKM LEETGTKVSI STVKRVLYRH NLKGHSARKK |
| 121 | PLLQNRHKKA RLRFATAHGD KDRTFWRNVL WSDETKIELF GHNDHRYVWR KKGEACKPKN |
| 181 | TIPTVKHGGG SIMLWGCFAA GGTGALHKID GIMDAVQYVD ILKQHLKTSV RKLKLGRKWV |
| 241 | FQHDNDPKHT SKVVAKWLKD NKVKVLEWPS QSPDLNPIEN LWAELKKRVR ARRPTNLTQL |
| 301 | HQLCQEEWAK IHPNYCGKLV EGYPKRLTQV KQFKGNATKY. |

In certain embodiments of the methods of the disclosure, the transposon is a Helraiser transposon. In certain embodiments of the Helraiser transposon sequence, the transposase is flanked by left and right terminal sequences termed LTS and RTS. In certain embodiments, these sequences terminate with a conserved 5′-TC/CTAG-3′ motif. In certain embodiments, a 19 bp palindromic sequence with the potential to form the hairpin termination structure is located 11 nucleotides upstream of the RTS and comprises the sequence

(SEQ ID NO: 14500)
GTGCACGAATTTCGTGCACCGGGCCACTAG.

In certain embodiments of the methods of the disclosure, and, in particular those embodiments wherein the transposon is a Helraiser transposon, the transposase enzyme is a Helitron transposase enzyme. In certain embodiments, the Helitron transposase enzyme of the disclosure comprises an amino acid sequence comprising:

| (SEQ ID NO: 14501) | |
|---|---|
| 1 | MSKEQLLIQR SSAAERCRRY RQKMSAEQRA SDLERRRRLQ QNVSEEQLLE KRRSEAEKQR |
| 61 | RHRQKMSKDQ RAFEVERRRW RRQNMSREQS STSTTNTGRN CLLSKNGVHE DAILEHSCGG |
| 121 | MTVRCEFCLS LNFSDEKPSD GKFTRCCSKG KVCPNDIHFP DYPAYLKRLM TNEDSDSKNF |
| 181 | MENIRSINSS FAFASMGANI ASPSGYGPYC FRIHGQVYHR TGTLHPSDGV SRKFAQLYIL |
| 241 | DTAEATSKRL AMPENQGCSE RLMININNLM HEINELTKSY KMLHEVEKEA QSEAAAKGIA |
| 301 | PTEVIMAIKY DRNSDPGRYN SPRVTEVAVI FRNEDGEPPF ERDLLIHCKP DPNNPNATKM |
| 361 | KQISILFPTL DAMTYPILFP HGEKGWGTDI ALRLRDNSVI DNNTRQNVRT RVTQMQYYGF |
| 421 | HLSVRDTFNP ILNAGKLTQQ FIVDSYSKME ANRINFIKAN QSKLRVEKYS GLMDYLKSRS |
| 481 | ENDNVPIGKM IILPSSFEGS PRNMQQRYQD AMAIVTKYGK PDLFITMTCN PKWADITNNL |
| 541 | QRWQKVENRP DLVARVFNIK LNALLNDICK FHLFGKVIAK IHVIEFQKRG LPHAHILLIL |
| 601 | DSESKLRSED DIDRIVKAEI PDEDQCPRLF QIVKSNMVHG PCGIQNPNSP CMENGKCSKG |
| 661 | YPKEFQNATI GNIDGYPKYK RRSGSTMSIG NKVVDNTWIV PYNPYLCLKY NCHINVEVCA |
| 721 | SIKSVKYLFK YIYKGHDCAN IQISEKNIIN HDEVQDFIDS RYVSAPEAVW RLFAMRMHDQ |
| 781 | SHAITRLAIH LPNDQNLYFH TDDFAEVLDR AKRHNSTLMA WFLLNREDSD ARNYYYWEIP |
| 841 | QHYVFNNSLW TKRRKGGNKV LGRLFTVSFR EPERYYLRLL LLHVKGAISF EDLRTVGGVT |
| 901 | YDTFHEAAKH RGLLLDDTIW KDTIDDAIIL NMPKQLRQLF AYICVFGCPS AADKLWDENK |
| 961 | SHFIEDFCWK LHRREGACVN CEMHALNEIQ EVFTLHGMKC SHFKLPDYPL LMNANTCDQL |
| 1021 | YEQQQAEVLI NSLNDEQLAA FQTITSAIED QTVHPKCFFL DGPGGSGKTY LYKVLTHYIR |
| 1081 | GRGGTVLPTA STGIAANLLL GGRTFHSQYK LPIPLNETSI SRLDIKSEVA KTIKKAQLLI |
| 1141 | IDECTMASSH AINAIDRLLR EIMNLNVAFG GKVLLLGGDF RQCLSIVPHA MRSAIVQTSL |
| 1201 | KYCNVWGCFR KLSLKTNMRS EDSAYSEWLV KLGDGKLDSS FHLGMDIIEI PHEMICNGSI |
| 1261 | IEATFGNSIS IDNIKNISKR AILCPKNEHV QKLNEEILDI LDGDFHTYLS DDSIDSTDDA |
| 1321 | EKENFPIEFL NSITPSGMPC HKLKLKVGAI IMLLRNLNSK WGLCNGTRFI IKRLRPNIIE |
| 1381 | AEVLTGSAEG EVVLIPRIDL SPSDTGLPFK LIRRQFPVMP AFAMTINKSQ GQTLDRVGIF |
| 1441 | LPEPVFAHGQ LYVAFSRVRR ACDVKVKVVN TSSQGKLVKH SESVFTLNVV YREILE. |

In certain embodiments of the methods of the disclosure, the transposon is a Tol2 transposon.

In certain embodiments of the methods of the disclosure, and, in particular those embodiments wherein the transposon is a Tol2 transposon, the transposase enzyme is a Tol2 transposase enzyme. In certain embodiments, the Tol2 transposase enzyme of the disclosure comprises an amino acid sequence comprising:

| (SEQ ID NO: 14502) | |
|---|---|
| 1 | MEEVCDSSAA ASSTVQNQPQ DQEHPWPYLR EFFSLSGVNK DSFKMKCVLC LPLNKEISAF |
| 61 | KSSPSNLRKH IERMHPNYLK NYSKLTAQKR KIGTSTHASS SKQLKVDSVF PVKHVSPVTV |
| 121 | NKAILRYIIQ GLHPFSTVDL PSFKELISTL QPGISVITRP TLRSKIAEAA LIMKQKVTAA |
| 181 | MSEVEWIATT TDCWTARRKS FIGVTAHWIN PGSLERHSAA LACKRLMGSH TFEVLASAMN |
| 241 | DIHSEYEIRD KVVCTTTDSG SNFMKAFRVF GVENNDIETE ARRCESDDTD SEGCGEGSDG |
| 301 | VEFQDASRVL DQDDGFEFQL PKHQKCACHL LNLVSSVDAQ KALSNEHYKK LYRSVFGKCQ |
| 361 | ALWNKSSRSA LAAEAVESES RLQLLRPNQT RWNSTFMAVD RILQICKEAG EGALRNICTS |
| 421 | LEVPMFNPAE MLFLTEWANT MRPVAKVLDI LQAETNTQLG WLLPSVHQLS LKLQRLHHSL |
| 481 | RYCDPLVDAL QQGIQTRFKH MFEDPEIiAA AILLPKFRTS WTNDETIIKR GMDYIRVHLE |
| 541 | PLDHKKELAN SSSDDEDFFA SLKPTTHEAS KELDGYLACV SDTRESLLTF PAICSLSIKT |
| 601 | NTPLPASAAC ERLFSTAGLL FSPKRARLDT NNFENQLLLK LNLRFYNFE. |

In certain embodiments of the methods of the disclosure, the piggyBac-like transposon comprises an amino acid sequence having at least 5%, 10%, 15%, 20%, 25%, 30%, 35%, 40%, 45%, 50%, 55%, 60%, 65%, 70%, 75%, 80%, 85%, 90%, 95% or any percentage in between of identity to the amino acid sequence of SEQ ID NO: 14487.

In certain embodiments of the methods of the disclosure, a vector comprises the recombinant and non-naturally occurring DNA sequence encoding the transposon. In some embodiments, the vector comprises any form of DNA and wherein the vector comprises at least 100 nucleotides (nts), 500 nts, 1000 nts, 1500 nts, 2000 nts, 2500 nts, 3000 nts, 3500 nts, 4000 nts, 4500 nts, 5000 nts, 6500 nts, 7000 nts, 7500 nts, 8000 nts, 8500 nts, 9000 nts, 9500 nts, 10,000 nts or any number of nucleotides in between. In some embodiments, the vector comprises single-stranded or double-stranded DNA. In some embodiments, the vector comprises circular DNA. In some embodiments, the vector is a plasmid vector. In some embodiments, the vector is a nanoplasmid vector. In some embodiments, the vector is a minicircle. In some embodiments, the vector comprises linear or linearized DNA. In some embodiments, the linear or linearized DNA is produced in vitro. In some embodiments, the linear or linearized DNA is a product of a restriction digest of a circular DNA. In some embodiments, the circular DNA is a plasmid vector, a nanoplasmid vector or a minicircle DNA vector. In some embodiments, the linear or linearized DNA is a product of a polymerase chain reaction (PCR). In some embodiments, the vector is a double-stranded Doggybone™ DNA sequence. In some embodiments, the Doggybone™ DNA sequence is produced by an enzymatic process that solely encodes an antigen expression cassette, comprising antigen, promoter, poly-A tail and telomeric ends.

In certain embodiments of the methods of the disclosure, the immune cell or the immune cell precursor is isolated or derived from a human. In certain embodiments, the immune cell or the immune cell precursor is isolated or derived from a non-human mammal. In certain embodiments, the non-human mammal is a rodent, a rabbit, a cat, a dog, a pig, a horse, a cow, a camel or a primate.

In certain embodiments of the methods of the disclosure, the recombinant and non-naturally occurring DNA sequence encoding a transposon further comprises a sequence encoding a chimeric antigen receptor or a portion thereof. In certain embodiments, the chimeric antigen receptor (CAR) comprises (a) an ectodomain comprising an antigen recognition region, (b) a transmembrane domain, and (c) an endodomain comprising at least one costimulatory domain. In certain embodiments, the antigen recognition region comprises one or more of an antibody or a fragment thereof a single chain antibody (scFv), a single domain antibody, an antibody mimetic, a protein scaffold, a Centyrin, a VHH, and a VH.

Chimeric antigen receptors (CARs) of the disclosure may comprise (a) an ectodomain comprising an antigen recognition region, (b) a transmembrane domain, and (c) an endodomain comprising at least one costimulatory domain. In certain embodiments, the ectodomain may further comprise a signal peptide. Alternatively, or in addition, in certain embodiments, the ectodomain may further comprise a hinge between the antigen recognition region and the transmembrane domain. In certain embodiments of the CARs of the disclosure, the signal peptide may comprise a sequence encoding a human CD2, CD3δ, CD3ε, CD3γ, CD3ζ, CD4, CD8α, CD19, CD28, 4-1BB or GM-CSFR signal peptide. In certain embodiments of the CARs of the disclosure, the signal peptide may comprise a sequence encoding a human CD8α signal peptide. In certain embodiments, the transmembrane domain may comprise a sequence encoding a human CD2, CD3δ, CD3ε, CD3γ, CD3ζ, CD4, CD8α, CD19, CD28, 4-1BB or GM-CSFR transmembrane domain. In certain embodiments of the CARs of the disclosure, the transmembrane domain may comprise a sequence encoding a human CD8α transmembrane domain. In certain embodiments of the CARs of the disclosure, the endodomain may comprise a human CD3 endodomain. In certain embodiments of the CARs of the disclosure, the at least one costimulatory domain may comprise a human 4-1BB, CD28, CD40, ICOS, MyD88, OX-40 intracellular segment, or any combination thereof. In certain embodiments of the CARs of the disclosure, the at least one costimulatory domain may comprise a CD28 and/or a 4-1BB costimulatory domain. In certain embodiments of the CARs of the disclosure, the hinge may comprise a sequence derived from a human CD8α, IgG4, and/or CD4 sequence. In certain embodiments of the CARs of the disclosure, the hinge may comprise a sequence derived from a human CD8α sequence.

In certain embodiments of the methods of the disclosure, the recombinant and non-naturally occurring DNA sequence encoding a transposon further comprises a sequence encoding a chimeric antigen receptor or a portion thereof. The portion of the sequence encoding a chimeric antigen receptor may encode an antigen recognition region. The antigen recognition region may comprise one or more complementarity determining region(s). The antigen recognition region may comprise an antibody, an antibody mimetic, a protein scaffold or a fragment thereof. In certain embodiments, the antibody is a chimeric antibody, a recombinant antibody, a humanized antibody or a human antibody. In certain embodiments, the antibody is affinity-tuned. Nonlimiting examples of antibodies of the disclosure include a single-chain variable fragment (scFv), a VHH, a single domain antibody (sdAB), a small modular immunopharmaceutical (SMIP) molecule, or a nanobody. In certain embodiments, the VHH is camelid. Alternatively, or in addition, in certain embodiments, the VHH is humanized. Nonlimiting examples of antibody fragments of the disclosure include a complementary determining region, a variable region, a heavy chain, a light chain, or any combination thereof. Nonlimiting examples of antibody mimetics of the disclosure include an affibody, an afflilin, an affimer, an affitin, an alphabody, an anticalin, and avimer, a DARPin, a Fynomer, a Kunitz domain peptide, or a monobody. Nonlimiting examples of protein scaffolds of the disclosure include a Centyrin.

In certain embodiments of the methods of the disclosure, the nucleic acid sequence encoding the transposase enzyme is a DNA sequence, and an amount of the DNA sequence encoding the transposase enzyme and an amount of the DNA sequence encoding the transposon is equal to or less than 10.0 μg per 100 μL of an electroporation or nucleofection reaction. In certain embodiments, a concentration of the amount of the DNA sequence encoding the transposase enzyme and an amount of the DNA sequence encoding the transposon in the electroporation or nucleofection reaction is equal to or less than 100 μg/mL.

In certain embodiments of the methods of the disclosure, the nucleic acid sequence encoding the transposase enzyme is a DNA sequence, and an amount of the DNA sequence encoding the transposase enzyme and an amount of the DNA sequence encoding the transposon is equal to or less than 7.5 μg per 100 μL of an electroporation or nucleofection reaction. In certain embodiments, a concentration of the amount of the DNA sequence encoding the transposase enzyme and an amount of the DNA sequence encoding the transposon in the electroporation or nucleofection reaction is equal to or less than 75 μg/mL.

In certain embodiments of the methods of the disclosure, the nucleic acid sequence encoding the transposase enzyme is a DNA sequence, and an amount of the DNA sequence encoding the transposase enzyme and an amount of the DNA sequence encoding the transposon is equal to or less than 6.0 μg per 100 μL of an electroporation or nucleofection reaction. In certain embodiments, a concentration of the amount of the DNA sequence encoding the transposase enzyme and an amount of the DNA sequence encoding the transposon in the electroporation or nucleofection reaction is equal to or less than 60 μg/mL. In certain embodiments, the transposase is a Sleeping Beauty transposase. In certain embodiments, the Sleeping Beauty transposase is a Sleeping Beauty 100X (SB100X) transposase.

In certain embodiments of the methods of the disclosure, the nucleic acid sequence encoding the transposase enzyme is a DNA sequence, and an amount of the DNA sequence encoding the transposase enzyme and an amount of the DNA sequence encoding the transposon is equal to or less than 5.0 μg per 100 μL of an electroporation or nucleofection reaction. In certain embodiments, a concentration of the amount of the DNA sequence encoding the transposase enzyme and an amount of the DNA sequence encoding the transposon in the electroporation or nucleofection reaction is equal to or less than 50 μg/mL.

In certain embodiments of the methods of the disclosure, the nucleic acid sequence encoding the transposase enzyme is a DNA sequence, and an amount of the DNA sequence encoding the transposase enzyme and an amount of the DNA sequence encoding the transposon is equal to or less than 2.5 μg per 100 μL of an electroporation or nucleofection reaction. In certain embodiments, a concentration of the amount of the DNA sequence encoding the transposase enzyme and an amount of the DNA sequence encoding the transposon in the electroporation or nucleofection reaction is equal to or less than 25 μg/mL.

In certain embodiments of the methods of the disclosure, the nucleic acid sequence encoding the transposase enzyme is a DNA sequence, and an amount of the DNA sequence encoding the transposase enzyme and an amount of the DNA sequence encoding the transposon is equal to or less than 1.67 μg per 100 μL of an electroporation or nucleofection reaction. In certain embodiments, a concentration of the amount of the DNA sequence encoding the transposase enzyme and an amount of the DNA sequence encoding the transposon in the electroporation or nucleofection reaction is equal to or less than 16.7 μg/mL. In certain embodiments, the transposase is a Super piggyBac (PB) transposase. In certain embodiments, the piggyBac transposase comprises an amino acid sequence comprising SEQ ID NO: 14487.

In certain embodiments of the methods of the disclosure, the transposase is a piggyBac transposase. In certain embodiments, the piggyBac transposase comprises an amino acid sequence comprising SEQ ID NO: 14487. In certain embodiments, the piggyBac transposase is a hyperactive variant and wherein the hyperactive variant comprises an amino acid substitution at one or more of positions 30, 165, 282 and 538 of SEQ ID NO: 14487. In certain embodiments, the amino acid substitution at position 30 of SEQ ID NO: 14487 is a substitution of a valine (V) for an isoleucine (I) (I30V). In certain embodiments, the amino acid substitution at position 165 of SEQ ID NO: 14487 is a substitution of a serine (S) for a glycine (G) (G165S). In certain embodiments, the amino acid substitution at position 282 of SEQ ID NO: 14487 is a substitution of a valine (V) for a methionine (M) (M282V). In certain embodiments, the amino acid substitution at position 538 of SEQ ID NO: 14487 is a substitution of a lysine (K) for an asparagine (N) (N538K).

In certain embodiments of the methods of the disclosure, the nucleic acid sequence encoding the transposase enzyme is a DNA sequence, and (b) wherein an amount of the DNA sequence encoding the transposase enzyme and an amount of the DNA sequence encoding the transposon is equal to or less than 1.67 μg per 100 μL of an electroporation or nucleofection reaction. In certain embodiments, a concentration of the amount of the DNA sequence encoding the transposase enzyme and the amount of the DNA sequence encoding the transposon in the electroporation or nucleofection reaction is equal to or less than 16.7 μg/mL. In certain embodiments, the transposase is a Super piggyBac (PB) transposase. In certain embodiments, the Super piggyBac (PB) transposase enzyme comprises an amino acid sequence at least 75% identical to:

(SEQ ID NO: 14484)
MGSSLDDEHILSALLQSDDELVGEDSDSEVSDHVSEDDVQSDTEEAFI
DEVHEVQPTSSGSEILDEQNVIEQPGSSLASNRILTLPQRTIRGKNKH
CWSTSKSTRRSRVSALNIVRSQRGPTRMCRNIYDPLLCFKLFFTDEII
SEIVKWTNAEISLKRRESMTSATFRDTNEDEIYAFFGILVMTAVRKDN
HMSTDDLFDRSLSMVYVSVMSRDRFDFLIRCLRMDDKSIRPTLRENDV
FTPVRKIWDLFIHQCIQNYTPGAHLTIDEQLLGFRGRCPFRVYIPNKP
SKYGIKILMMCDSGTKYMINGMPYLGRGTQTNGVPLGEYYVKELSKPV
HGSCRNITCDNWFTSIPLAKNLLQEPYKLTIVGTVRSNKREIPEVLKN
SRSRPVGTSMFCFDGPLTLVSYKPKPAKMVYLLSSCDEDASINESTGK
PQMVMYYNQTKGGVDTLDQMCSVMTCSRKTNRWPMALLYGMINIACIN
SFIIYSHNVSSKGEKVQSRKKFMRNLYMSLTSSFMRKRLEAPTLKRYL
RDNISNILPKEVPGTSDDSTEEPVMKKRTYCTYCPSKIRRKANASCKK
CKKVICREHNIDMCQSCF.

In certain embodiments of the methods of the disclosure, the nucleic acid sequence encoding the transposase enzyme is a DNA sequence, and an amount of the DNA sequence encoding the transposase enzyme and an amount of the DNA sequence encoding the transposon is equal to or less than 0.55 μg per 100 μL of an electroporation or nucleofection reaction. In certain embodiments, a concentration of the amount of the DNA sequence encoding the transposase enzyme and an amount of the DNA sequence encoding the transposon in the electroporation or nucleofection reaction is equal to or less than 5.5 μg/mL.

In certain embodiments of the methods of the disclosure, the nucleic acid sequence encoding the transposase enzyme is a DNA sequence, and an amount of the DNA sequence encoding the transposase enzyme and an amount of the DNA sequence encoding the transposon is equal to or less than 0.19 μg per 100 μL of an electroporation or nucleofection reaction. In certain embodiments, a concentration of the amount of the DNA sequence encoding the transposase enzyme and an amount of the DNA sequence encoding the transposon in the electroporation or nucleofection reaction is equal to or less than 1.9 μg/mL.

In certain embodiments of the methods of the disclosure, the nucleic acid sequence encoding the transposase enzyme is a DNA sequence, and an amount of the DNA sequence encoding the transposase enzyme and an amount of the DNA sequence encoding the transposon is equal to or less than 0.10 μg per 100 μL of an electroporation or nucleofection reaction. In certain embodiments, a concentration of the amount of the DNA sequence encoding the transposase enzyme and an amount of the DNA sequence encoding the transposon in the electroporation or nucleofection reaction is equal to or less than 1.0 μg/mL.

In certain embodiments of the methods of the disclosure, the nucleic acid sequence encoding the transposase enzyme is a RNA sequence, and an amount of the DNA sequence encoding the transposon is equal to or less than 10.0 μg per 100 μL of an electroporation or nucleofection reaction. In certain embodiments, a concentration of the amount of the DNA sequence encoding the transposon in the electroporation or nucleofection reaction is equal to or less than 100 μg/mL.

In certain embodiments of the methods of the disclosure, the nucleic acid sequence encoding the transposase enzyme is a RNA sequence, and an amount of the DNA sequence encoding the transposon is equal to or less than 7.5 μg per 100 μL of an electroporation or nucleofection reaction. In certain embodiments, a concentration of the amount of the DNA sequence encoding the transposon in the electroporation or nucleofection reaction is equal to or less than 75 μg/mL.

In certain embodiments of the methods of the disclosure, the nucleic acid sequence encoding the transposase enzyme is a RNA sequence, and an amount of the DNA sequence encoding the transposon is equal to or less than 6.0 μg per 100 μL of an electroporation or nucleofection reaction. In certain embodiments, a concentration of the amount of the DNA sequence encoding the transposon in the electroporation or nucleofection reaction is equal to or less than 60 μg/mL. In certain embodiments, the transposase is a Sleeping Beauty transposase. In certain embodiments, the Sleeping Beauty transposase is a Sleeping Beauty 100X (SB100X) transposase.

In certain embodiments of the methods of the disclosure, the nucleic acid sequence encoding the transposase enzyme is a RNA sequence, and an amount of the DNA sequence encoding the transposon is equal to or less than 5.0 μg per 100 μL of an electroporation or nucleofection reaction. In certain embodiments, a concentration of the amount of the DNA sequence encoding the transposon in the electroporation or nucleofection reaction is equal to or less than 50 μg/mL.

In certain embodiments of the methods of the disclosure, the nucleic acid sequence encoding the transposase enzyme is a RNA sequence, and an amount of the DNA sequence encoding the transposon is equal to or less than 2.5 μg per 100 μL of an electroporation or nucleofection reaction. In certain embodiments, a concentration of the amount of the DNA sequence encoding the transposon in the electroporation or nucleofection reaction is equal to or less than 25 μg/mL.

In certain embodiments of the methods of the disclosure, the nucleic acid sequence encoding the transposase enzyme is a RNA sequence, and an amount of the DNA sequence encoding the transposon is equal to or less than 1.67 μg per 100 μL of an electroporation or nucleofection reaction. In certain embodiments, a concentration of the amount of the DNA sequence encoding the transposon in the electroporation or nucleofection reaction is equal to or less than 16.7 μg/mL. In certain embodiments, the transposase is a Super piggyBac (PB) transposase.

In certain embodiments of the methods of the disclosure, the nucleic acid sequence encoding the transposase enzyme is a RNA sequence, and an amount of the DNA sequence encoding the transposon is equal to or less than 0.55 μg per 100 μL of an electroporation or nucleofection reaction. In certain embodiments, a concentration of the amount of the DNA sequence encoding the transposon in the electroporation or nucleofection reaction is equal to or less than 5.5 μg/mL.

In certain embodiments of the methods of the disclosure, the nucleic acid sequence encoding the transposase enzyme is a RNA sequence, and an amount of the DNA sequence encoding the transposon is equal to or less than 0.19 μg per 100 μL of an electroporation or nucleofection reaction. In certain embodiments, a concentration of the amount of the DNA sequence encoding the transposon in the electroporation or nucleofection reaction is equal to or less than 1.9 μg/mL.

In certain embodiments of the methods of the disclosure, the nucleic acid sequence encoding the transposase enzyme is a RNA sequence, and an amount of the DNA sequence encoding the transposon is equal to or less than 0.1 μg per 100 μL of an electroporation or nucleofection reaction. In certain embodiments, a concentration of the amount of the DNA sequence encoding the transposon in the electroporation or nucleofection reaction is equal to or less than 1.0 μg/mL.

The disclosure provides an immune cell modified according to the method of the disclosure. The immune cell may be a T-lymphocyte, a Natural Killer (NK) cell, a Cytokine-induced Killer (CIK) cell or a Natural Killer T (NKT) cell. The immune cell may be further modified by a second gene editing tool, including, but not limited to those gene editing tools comprising an endonuclease operably-linked to either a Cas9 or a TALE sequence. In certain embodiments of the second gene editing tool, the endonuclease is operably-linked to either a Cas9 or a TALE sequence covalently. In certain embodiments of the second gene editing tool, the endonuclease is operably-linked to either a Cas9 or a TALE sequence non-covalently. In certain embodiments, the endonuclease comprises a Clo051 domain. In certain embodiments, Clo051 domain comprises a sequence of

(SEQ ID NO: 14503)
EGIKSNISLLKDELRGQISHISHEYLSLIDLAFDSKQNRLFEMKVLEL
LVNEYGFKGRHLGGSRKPDGIVYSTTLEDNFGIIVDTKAYSEGYSLPI
SQADEMERYVRENSNRDEEVNPNKWWENFSEEVKKYYFVFISGSFKGK
FEEQLRRLSMTTGVNGSAVNVVNLLLGAEKIRSGEMTIEELERAMFNN
SEFILKY.

In certain embodiments, the Cas9 is an inactivated Cas9 (dCas9). In certain embodiments, the inactivated Cas9 is isolated or derived from Staphylococcus aureus and comprises D10A and N580A within the catalytic site. In certain embodiments, the Cas9 is a small and inactivated Cas9 (dSaCas9). In certain embodiments, the dSaCas9 comprises the amino acid sequence of

| (SEQ ID NO: 14497) | |
|---|---|
| 1 | MKRNYILGLA IGITSVGYGI IDYETRDVID AGVRLFKEAN VENNEGRRSK RGARRLKRRR |
| 61 | RHRIQRVKKL LFDYNLLTDH SELSGINPYE ARVKGLSQKL SEEEFSAALL HLAKRRGVHN |
| 121 | VNEVEEDTGN ELSTKEQISR NSKALEEKYV AELQLERLKK DGEVRGSINR FKTSDYVKEA |
| 181 | KQLLKVQKAY HQLDQSFIDT YIDLLETRRT YYEGPGEGSP FGWKDIKEWY EMLMGHCTYF |
| 241 | PEELRSVKYA YNADLYNALN DLNNLVITRD ENEKLEYYEK FQIIENVFKQ KKKPTLKQIA |
| 301 | KEILVNEEDI KGYRVTSTGK PEFTNLKVYH DIKDITARKE IIENAELLDQ IAKILTIYQS |
| 361 | SEDIQEELTN LNSELTQEEI EQISNLKGYT GTHNLSLKAI NLILDELWHT NDNQIAIFNR |
| 421 | LKLVPKKVDL SQQKEIPTTL VDDFILSPVV KRSFIQSIKV INAIIKKYGL PNDIIIELAR |
| 481 | EKNSKDAQKM INEMQKRNRQ TNERIEEIIR TTGKENAKYL IEKIKLHDMQ EGKCLYSLEA |
| 541 | IPLEDLLNNP FNYEVDHIIP RSVSFDNSFN NKVLVKQEEA SKKGNRTPFQ YLSSSDSKIS |
| 601 | YETFKKHILN LAKGKGRISK TKKEYLLEER DINRFSVQKD FINRNLVDTR YATRGLMNLL |
| 661 | RSYFRVNNLD VKVKSINGGF TSFLRRKWKF KKERNKGYKH HAEDALIIAN ADFIFKEWKK |
| 721 | LDKAKKVMEN QMFEEKQAES MPEIETEQEY KEIFITPHQI KHIKDFKDYK YSHRVDKKPN |
| 781 | RELINDTLYS TRKDDKGNTL IVNNLNGLYD KDNDKLKKLI NKSPEKLLMY HHDPQTYQKL |
| 841 | KLIMEQYGDE KNPLYKYYEE TGNYLTKYSK KDNGPVIKKI KYYGNKLNAH LDITDDYPNS |
| 901 | RNKVVKLSLK PYRFDVYLDN GVYKFVTVKN LDVIKKENYY EVNSKCYEEA KKLKKISNQA |
| 961 | EFIASFYNND LIKINGELYR VIGVNNDLLN RIEVNMIDIT YREYLENMND KRPPRIIKTI |
| 1021 | ASKTQSIKKY STDILGNLYE VKSKKHPQII KKG. |

In certain embodiments, the Cas9 is an inactivated Cas9 (dCas9). In certain embodiments, the inactivated Cas9 (dCas9) is isolated or derived from Staphylococcus pyogenes and comprises D10A and H840A within the catalytic site. In certain embodiments, the dCas9 comprises the amino acid sequence of:

| (SEQ ID NO: 14498) | |
|---|---|
| 1 | XDKKYSIGLA IGTNSVGWAV ITDEYKVPSK KFKVLGNTDR HSIKKNLIGA LLFDSGETAE |
| 61 | ATRLKRTARR RYTRRKNRIC YLQEIFSNEM AKVDDSFFHR LEESFLVEED KKHERHPIFG |
| 121 | NIVDEVAYHE KYPTIYHLRK KLVDSTDKAD LRLIYLALAH MIKFRGHFLI EGDLNPDNSD |
| 181 | VDKLFIQLVQ TYNQLFEENP INASGVDAKA ILSARLSKSR RLENLIAQLP GEKKNGLFGN |
| 241 | LIALSLGLTP NFKSNFDLAE DAKLQLSKDT YDDDLDNLLA QIGDQYADLF LAAKNLSDAI |
| 301 | LLSDILRVNT EITKAPLSAS MIKRYDEHHQ DLTLLKALVR QQLPEKYKEI FFDQSKNGYA |
| 361 | GYIDGGASQE EFYKFIKPIL EKMDGTEELL VKLNREDLLR KQRTFDNGSI PHQIHLGELH |
| 421 | AILRRQEDFY PFLKDNREKI EKILTFRIPY YVGPLARGNS RFAWMTRKSE ETITPWNFEE |
| 481 | VVDKGASAQS FIERMTNFDK NLPNEKVLPK HSLLYEYFTV YNELTKVKYV TEGMRKPAFL |
| 541 | SGEQKKAIVD LLFKTNRKVT VKQLKEDYFK KIECFDSVEI SGVEDRFNAS LGTYHDLLKI |
| 601 | IKDKDFLDNE ENEDILEDIV LTLTLFEDRE MIEERLKTYA HLFDDKVMKQ LKRRRYTGWG |
| 661 | RLSRKLINGI RDKQSGKTIL DFLKSDGFAN RNFMQLIHDD SLTFKEDIQK AQVSGQGDSL |
| 721 | HEHIANLAGS PAIKKGILQT VKVVDELVKV MGRHKPENIV IEMARENQTT QKGQKNSRER |
| 781 | MKRIEEGIKE LGSQILKEHP VENTQLQNEK LYLYYLQNGR DMYVDQELDI NRLSDYDVDA |
| 841 | IVPQSFLKDD SIDNKVLTRS DKNRGKSDNV PSEEVVKKMK NYWRQLLNAK LITQRKFDNL |
| 901 | TKAERGGLSE LDKAGFIKRQ LVETRQITKH VAQILDSRMN TKYDENDKLI REVKVITLKS |
| 961 | KLVSDFRKDF QFYKVREINN YHHAHDAYLN AVVGTALIKK YPKLESEFVY GDYKVYDVRK |
| 1021 | MIAKSEQEIG KATAKYFFYS NIMNFFKTEI TLANGEIRKR PLIETNGETG EIVWDKGRDF |
| 1081 | ATVRKVLSMP QVNIVKKTEV QTGGFSKESI LPKRNSDKLI ARKKDWDPKK YGGFDSPTVA |
| 1141 | YSVLVVAKVE KGKSKKLKSV KELLGITIME RSSFEKNPID FLEAKGYKEV KKDLIIKLPK |
| 1201 | YSLFELENGR KRMLASAGEL QKGNELALPS KYVNFLYLAS HYEKLKGSPE DNEQKQLFVE |
| 1261 | QHKHYLDEII EQISEFSKRV ILADANLDKV LSAYNKHRDK PIREQAENII HLFTLTNLGA |
| 1321 | PAAFKYFDTT IDRKRYTSTK EVLDATLIHQ SITGLYETRI DLSQLGGD. |

In certain embodiments, the Cas9 is an inactivated Cas9 (dCas9). In certain embodiments, the inactivated Cas9 (dCas9) is isolated or derived from Staphylococcus pyogenes and comprises D10A and H840A within the catalytic site. In certain embodiments, the dCas9 comprises the amino acid sequence of:

| (SEQ ID NO: 14499) | |
|---|---|
| 1 | MDKKYSIGLA IGTNSVGWAV ITDEYKVPSK KFKVLGNTDR HSIKKNLIGA LLFDSGETAE |
| 61 | ATRLKRTARR RYTRRKNRIC YLQEIFSNEM AKVDDSFFHR LEESFLVEED KKHERHPIFG |
| 121 | NIVDEVAYHE KYPTIYHLRK KLVDSTDKAD LRLIYLALAH MIKFRGHFLI EGDLNPDNSD |
| 181 | VDKLFIQLVQ TYNQLFEENP INASGVDAKA ILSARLSKSR RLENLIAQLP GEKKNGLFGN |
| 241 | LIALSLGLTP NFKSNFDLAE DAKLQLSKDT YDDDLDNLLA QIGDQYADLF LAAKNLSDAI |
| 301 | LLSDILRVNT EITKAPLSAS MIKRYDEHHQ DLTLLKALVR QQLPEKYKEI FFDQSKNGYA |
| 361 | GYIDGGASQE EFYKFIKPIL EKMDGTEELL VKLNREDLLR KQRTFDNGSI PHQIHLGELH |
| 421 | AILRRQEDFY PFLKDNREKI EKILTFRIPY YVGPLARGNS RFAWMTRKSE ETITPWNFEE |
| 481 | VVDKGASAQS FIERMTNFDK NLPNEKVLPK HSLLYEYFTV YNELTKVKYV TEGMRKPAFL |
| 541 | SGEQKKAIVD LLFKTNRKVT VKQLKEDYFK KIECFDSVEI SGVEDRFNAS LGTYHDLLKI |
| 601 | IKDKDFLDNE ENEDILEDIV LTLTLFEDRE MIEERLKTYA HLFDDKVMKQ LKRRRYTGWG |
| 661 | RLSRKLINGI RDKQSGKTIL DFLKSDGFAN RNFMQLIHDD SLTFKEDIQK AQVSGQGDSL |
| 721 | HEHIANLAGS PAIKKGILQT VKVVDELVKV MGRHKPENIV IEMARENQTT QKGQKNSRER |
| 781 | MKRIEEGIKE LGSQILKEHP VENTQLQNEK LYLYYLQNGR DMYVDQELDI NRLSDYDVDA |
| 841 | IVPQSFLKDD SIDNKVLTRS DKNRGKSDNV PSEEVVKKMK NYWRQLLNAK LITQRKFDNL |
| 901 | TKAERGGLSE LDKAGFIKRQ LVETRQITKH VAQILDSRMN TKYDENDKLI REVKVITLKS |
| 961 | KLVSDFRKDF QFYKVREINN YHHAHDAYLN AVVGTALIKK YPKLESEFVY GDYKVYDVRK |
| 1021 | MIAKSEQEIG KATAKYFFYS NIMNFFKTEI TLANGEIRKR PLIETNGETG EIVWDKGRDF |
| 1081 | ATVRKVLSMP QVNIVKKTEV QTGGFSKESI LPKRNSDKLI ARKKDWDPKK YGGFDSPTVA |
| 1141 | YSVLVVAKVE KGKSKKLKSV KELLGITIME RSSFEKNPID FLEAKGYKEV KKDLIIKLPK |
| 1201 | YSLFELENGR KRMLASAGEL QKGNELALPS KYVNFLYLAS HYEKLKGSPE DNEQKQLFVE |
| 1261 | QHKHYLDEII EQISEFSKRV ILADANLDKV LSAYNKHRDK PIREQAENII HLFTLTNLGA |
| 1321 | PAAFKYFDTT IDRKRYTSTK EVLDATLIHQ SITGLYETRI DLSQLGGD. |

The disclosure provides an immune cell modified according to the method of the disclosure. The immune cell may be a T-lymphocyte, a Natural Killer (NK) cell, a Cytokine-induced Killer (CIK) cell or a Natural Killer T (NKT) cell. The immune cell may be further modified by a second gene editing tool, including, but not limited to those gene editing tools comprising an endonuclease operably-linked to either a Cas9 or a TALE sequence. Alternatively or in addition, the second gene editing tool may include an excision-only piggyBac transposase to re-excise the inserted sequences or any portion thereof. For example, the excision-only piggyBac transposase may be used to “re-excise” the transposon.

In certain embodiments, the transposon is a piggyBac transposon. In certain embodiments, and, in particular, those embodiments wherein the transposon is a piggyBac transposon, the transposase is a piggyBac™ or a Super piggyBac™ (SPB) transposase. In certain embodiments, and, in particular, those embodiments wherein the transposase is a Super piggyBac™ (SPB) transposase, the sequence encoding the transposase is an mRNA sequence.

In certain embodiments of the methods of the disclosure, the transposase enzyme is a piggyBac™ (PB) transposase enzyme. The piggyBac (PB) transposase enzyme may comprise or consist of an amino acid sequence at least 75%, 80%, 85%, 90%, 95%, 99% or any percentage in between identical to:

(SEQ ID NO: 14487)

| | |
|---|---|
| 1 | MGSSLDDEHI LSALLQSDDE LVGEDSDSEI SDHVSEDDVQ |
| | SDTEEAFIDE VHEVQPTSSG |
| 61 | SEILDEQNVI EQPGSSLASN RILTLPQRTI RGKNKHCWST |
| | SKSTRRSRVS ALNIVRSQRG |
| 121 | PTRMCRNIYD PLLCFKLFFT DEIISEIVKW TNAEISLKRR |
| | ESMTGATFRD TNEDEIYAFF |
| 181 | GILVMTAVRK DNHMSTDDLF DRSLSMVYVS VMSRDRFDFL |
| | IRCLRMDDKS IRPTLRENDV |
| 241 | FTPVRKIWDL FIHQCIQNYT PGAHLTIDEQ LLGFRGRCPF |
| | RMYIPNKPSK YGIKILMMCD |
| 301 | SGTKYMINGM PYLGRGTQTN GVPLGEYYVK ELSKPVHGSC |
| | RNITCDNWFT SIPLAKNLLQ |
| 361 | EPYKLTIVGT VRSNKREIPE VLKNSRSRPV GTSMFCFDGP |
| | LTLVSYKPKP AKMVYLLSSC |
| 421 | DEDASINEST GKPQMVMYYN QTKGGVDTLD QMCSVMTCSR |
| | KTNRWPMALL YGMINIACIN |
| 481 | SFIIYSHNVS SKGEKVQSRK KFMRNLYMSL TSSFMRKRLE |
| | APTLKRYLRD NISNILPNEV |
| 541 | PGTSDDSTEE PVMKKRTYCT YCPSKIRRKA NASCKKCKKV |
| | ICREHNIDMC QSCF. |

In certain embodiments of the methods of the disclosure, the transposase enzyme is a piggyBac™ (PB) transposase enzyme that comprises or consists of an amino acid sequence having an amino acid substitution at one or more of positions 30, 165, 282, or 538 of the sequence:

(SEQ ID NO: 14487)

| | |
|---|---|
| 1 | MGSSLDDEHI LSALLQSDDE LVGEDSDSEI SDHVSEDDVQ |
| | SDTEEAFIDE VHEVQPTSSG |
| 61 | SEILDEQNVI EQPGSSLASN RILTLPQRTI RGKNKHCWST |
| | SKSTRRSRVS ALNIVRSQRG |
| 121 | PTRMCRNIYD PLLCFKLFFT DEIISEIVKW TNAEISLKRR |
| | ESMTGATFRD TNEDEIYAFF |
| 181 | GILVMTAVRK DNHMSTDDLF DRSLSMVYVS VMSRDRFDFL |
| | IRCLRMDDKS IRPTLRENDV |
| 241 | FTPVRKIWDL FIHQCIQNYT PGAHLTIDEQ LLGFRGRCPF |
| | RMYIPNKPSK YGIKILMMCD |
| 301 | SGTKYMINGM PYLGRGTQTN GVPLGEYYVK ELSKPVHGSC |
| | RNITCDNWFT SIPLAKNLLQ |
| 361 | EPYKLTIVGT VRSNKREIPE VLKNSRSRPV GTSMFCFDGP |
| | LTLVSYKPKP AKMVYLLSSC |
| 421 | DEDASINEST GKPQMVMYYN QTKGGVDTLD QMCSVMTCSR |
| | KTNRWPMALL YGMINIACIN |
| 481 | SFIIYSHNVS SKGEKVQSRK KFMRNLYMSL TSSFMRKRLE |
| | APTLKRYLRD NISNILPNEV |
| 541 | PGTSDDSTEE PVMKKRTYCT YCPSKIRRKA NASCKKCKKV |
| | ICREHNIDMC QSCF. |

In certain embodiments, the transposase enzyme is a piggyBac™ (PB) transposase enzyme that comprises or consists of an amino acid sequence having an amino acid substitution at two or more of positions 30, 165, 282, or 538 of the sequence of SEQ ID NO: 14487. In certain embodiments, the transposase enzyme is a piggyBac™ (PB) transposase enzyme that comprises or consists of an amino acid sequence having an amino acid substitution at three or more of positions 30, 165, 282, or 538 of the sequence of SEQ ID NO: 14487. In certain embodiments, the transposase enzyme is a piggyBac™ (PB) transposase enzyme that comprises or consists of an amino acid sequence having an amino acid substitution at each of the following positions 30, 165, 282, and 538 of the sequence of SEQ ID NO: 14487. In certain embodiments, the amino acid substitution at position 30 of the sequence of SEQ ID NO: 14487 is a substitution of a valine (V) for an isoleucine (I). In certain embodiments, the amino acid substitution at position 165 of the sequence of SEQ ID NO: 14487 is a substitution of a serine (S) for a glycine (G). In certain embodiments, the amino acid substitution at position 282 of the sequence of SEQ ID NO: 14487 is a substitution of a valine (V) for a methionine (M). In certain embodiments, the amino acid substitution at position 538 of the sequence of SEQ ID NO: 14487 is a substitution of a lysine (K) for an asparagine (N).

In certain embodiments of the methods of the disclosure, the transposase enzyme is a Super piggyBac™ (SPB) transposase enzyme. In certain embodiments, the Super piggyBac™ (SPB) transposase enzymes of the disclosure may comprise or consist of the amino acid sequence of the sequence of SEQ ID NO: 14487 wherein the amino acid substitution at position 30 is a substitution of a valine (V) for an isoleucine (I), the amino acid substitution at position 165 is a substitution of a serine (S) for a glycine (G), the amino acid substitution at position 282 is a substitution of a valine (V) for a methionine (M), and the amino acid substitution at position 538 is a substitution of a lysine (K) for an asparagine (N). In certain embodiments, the Super piggyBac™ (SPB) transposase enzyme may comprise or consist of an amino acid sequence at least 75%, 80%, 85%, 90%, 95%, 99% or any percentage in between identical to:

(SEQ ID NO: 14484)

| | |
|---|---|
| 1 | MGSSLDDEHI LSALLQSDDE LVGEDSDSEV SDHVSEDDVQ |
| | SDTEEAFIDE VHEVQPTSSG |
| 61 | SEILDEQNVI EQPGSSLASN RILTLPQRTI RGKNKHCWST |
| | SKSTRRSRVS ALNIVRSQRG |
| 121 | PTRMCRNIYD PLLCFKLFFT DEIISEIVKW TNAEISLKRR |
| | ESMTSATFRD TNEDEIYAFF |
| 181 | GILVMTAVRK DNHMSTDDLF DRSLSMVYVS VMSRDRFDFL |
| | IRCLRMDDKS IRPTLRENDV |
| 241 | FTPVRKIWDL FIHQCIQNYT PGAHLTIDEQ LLGFRGRCPF |
| | RVYIPNKPSK YGIKILMMCD |
| 301 | SGTKYMINGM PYLGRGTQTN GVPLGEYYVK ELSKPVHGSC |
| | RNITCDNWFT SIPLAKNLLQ |
| 361 | EPYKLTIVGT VRSNKREIPE VLKNSRSRPV GTSMFCFDGP |
| | LTLVSYKPKP AKMVYLLSSC |
| 421 | DEDASINEST GKPQMVMYYN QTKGGVDTLD QMCSVMTCSR |
| | KTNRWPMALL YGMINIACIN |
| 481 | SFIIYSHNVS SKGEKVQSRK KFMRNLYMSL TSSFMRKRLE |
| | APTLKRYLRD NISNILPKEV |
| 541 | PGTSDDSTEE PVMKKRTYCT YCPSKIRRKA NASCKKCKKV |
| | ICREHNIDMC QSCF. |

In certain embodiments of the methods of the disclosure, including those embodiments wherein the transposase comprises the above-described mutations at positions 30, 165, 282 and/or 538, the piggyBac™ or Super piggyBac™ transposase enzyme may further comprise an amino acid substitution at one or more of positions 3, 46, 82, 103, 119, 125, 177, 180, 185, 187, 200, 207, 209, 226, 235, 240, 241, 243, 258, 296, 298, 311, 315, 319, 327, 328, 340, 421, 436, 456, 470, 486, 503, 552, 570 and 591 of the sequence of SEQ ID NO: 14487 or SEQ ID NO: 14484. In certain embodiments, including those embodiments wherein the transposase comprises the above-described mutations at positions 30, 165, 282 and/or 538, the piggyBac™ or Super piggyBac™ transposase enzyme may further comprise an amino acid substitution at one or more of positions 46, 119, 125, 177, 180, 185, 187, 200, 207, 209, 226, 235, 240, 241, 243, 296, 298, 311, 315, 319, 327, 328, 340, 421, 436, 456, 470, 485, 503, 552 and 570. In certain embodiments, the amino acid substitution at position 3 of SEQ ID NO: 14487 or SEQ ID NO: 14484 is a substitution of an asparagine (N) for a serine (S). In certain embodiments, the amino acid substitution at position 46 of SEQ ID NO: 14487 or SEQ ID NO: 14484 is a substitution of a serine (S) for an alanine (A). In certain embodiments, the amino acid substitution at position 46 of SEQ ID NO: 14487 or SEQ ID NO: 14484 is a substitution of a threonine (T) for an alanine (A). In certain embodiments, the amino acid substitution at position 82 of SEQ ID NO: 14487 or SEQ ID NO: 14484 is a substitution of a tryptophan (W) for an isoleucine (I). In certain embodiments, the amino acid substitution at position 103 of SEQ ID NO: 14487 or SEQ ID NO: 14484 is a substitution of a proline (P) for a serine (S). In certain embodiments, the amino acid substitution at position 119 of SEQ ID NO: 14487 or SEQ ID NO: 14484 is a substitution of a proline (P) for an arginine (R). In certain embodiments, the amino acid substitution at position 125 of SEQ ID NO: 14487 or SEQ ID NO: 14484 is a substitution of an alanine (A) a cysteine (C). In certain embodiments, the amino acid substitution at position 125 of SEQ ID NO: 14487 or SEQ ID NO: 14484 is a substitution of a leucine (L) for a cysteine (C). In certain embodiments, the amino acid substitution at position 177 of SEQ ID NO: 14487 or SEQ ID NO: 14484 is a substitution of a lysine (K) for a tyrosine (Y). In certain embodiments, the amino acid substitution at position 177 of SEQ ID NO: 14487 or SEQ ID NO: 14484 is a substitution of a histidine (H) for a tyrosine (Y). In certain embodiments, the amino acid substitution at position 180 of SEQ ID NO: 14487 or SEQ ID NO: 14484 is a substitution of a leucine (L) for a phenylalanine (F). In certain embodiments, the amino acid substitution at position 180 of SEQ ID NO: 14487 or SEQ ID NO: 14484 is a substitution of an isoleucine (I) for a phenylalanine (F). In certain embodiments, the amino acid substitution at position 180 of SEQ ID NO: 14487 or SEQ ID NO: 14484 is a substitution of a valine (V) for a phenylalanine (F). In certain embodiments, the amino acid substitution at position 185 of SEQ ID NO: 14487 or SEQ ID NO: 14484 is a substitution of a leucine (L) for a methionine (M). In certain embodiments, the amino acid substitution at position 187 of SEQ ID NO: 14487 or SEQ ID NO: 14484 is a substitution of a glycine (G) for an alanine (A). In certain embodiments, the amino acid substitution at position 200 of SEQ ID NO: 14487 or SEQ ID NO: 14484 is a substitution of a tryptophan (W) for a phenylalanine (F). In certain embodiments, the amino acid substitution at position 207 of SEQ ID NO: 14487 or SEQ ID NO: 14484 is a substitution of a proline (P) for a valine (V). In certain embodiments, the amino acid substitution at position 209 of SEQ ID NO: 14487 or SEQ ID NO: 14484 is a substitution of a phenylalanine (F) for a valine (V). In certain embodiments, the amino acid substitution at position 226 of SEQ ID NO: 14487 or SEQ ID NO: 14484 is a substitution of a phenylalanine (F) for a methionine (M). In certain embodiments, the amino acid substitution at position 235 of SEQ ID NO: 14487 or SEQ ID NO: 14484 is a substitution of an arginine (R) for a leucine (L). In certain embodiments, the amino acid substitution at position 240 of SEQ ID NO: 14487 or SEQ ID NO: 14484 is a substitution of a lysine (K) for a valine (V). In certain embodiments, the amino acid substitution at position 241 of SEQ ID NO: 14487 or SEQ ID NO: 14484 is a substitution of a leucine (L) for a phenylalanine (F). In certain embodiments, the amino acid substitution at position 243 of SEQ ID NO: 14487 or SEQ ID NO: 14484 is a substitution of a lysine (K) for a proline (P). In certain embodiments, the amino acid substitution at position 258 of SEQ ID NO: 14487 or SEQ ID NO: 14484 is a substitution of a serine (S) for an asparagine (N). In certain embodiments, the amino acid substitution at position 296 of SEQ ID NO: 14487 or SEQ ID NO: 14484 is a substitution of a tryptophan (W) for a leucine (L). In certain embodiments, the amino acid substitution at position 296 of SEQ ID NO: 14487 or SEQ ID NO: 14484 is a substitution of a tyrosine (Y) for a leucine (L). In certain embodiments, the amino acid substitution at position 296 of SEQ ID NO: 14487 or SEQ ID NO: 14484 is a substitution of a phenylalanine (F) for a leucine (L). In certain embodiments, the amino acid substitution at position 298 of SEQ ID NO: 14487 or SEQ ID NO: 14484 is a substitution of a leucine (L) for a methionine (M). In certain embodiments, the amino acid substitution at position 298 of SEQ ID NO: 14487 or SEQ ID NO: 14484 is a substitution of an alanine (A) for a methionine (M). In certain embodiments, the amino acid substitution at position 298 of SEQ ID NO: 14487 or SEQ ID NO: 14484 is a substitution of a valine (V) for a methionine (M). In certain embodiments, the amino acid substitution at position 311 of SEQ ID NO: 14487 or SEQ ID NO: 14484 is a substitution of an isoleucine (I) for a proline (P). In certain embodiments, the amino acid substitution at position 311 of SEQ ID NO: 14487 or SEQ ID NO: 14484 is a substitution of a valine for a proline (P). In certain embodiments, the amino acid substitution at position 315 of SEQ ID NO: 14487 or SEQ ID NO: 14484 is a substitution of a lysine (K) for an arginine (R). In certain embodiments, the amino acid substitution at position 319 of SEQ ID NO: 14487 or SEQ ID NO: 14484 is a substitution of a glycine (G) for a threonine (T). In certain embodiments, the amino acid substitution at position 327 of SEQ ID NO: 14487 or SEQ ID NO: 14484 is a substitution of an arginine (R) for a tyrosine (Y). In certain embodiments, the amino acid substitution at position 328 of SEQ ID NO: 14487 or SEQ ID NO: 14484 is a substitution of a valine (V) for a tyrosine (Y). In certain embodiments, the amino acid substitution at position 340 of SEQ ID NO: 14487 or SEQ ID NO: 14484 is a substitution of a glycine (G) for a cysteine (C). In certain embodiments, the amino acid substitution at position 340 of SEQ ID NO: 14487 or SEQ ID NO: 14484 is a substitution of a leucine (L) for a cysteine (C). In certain embodiments, the amino acid substitution at position 421 of SEQ ID NO: 14487 or SEQ ID NO: 14484 is a substitution of a histidine (H) for the aspartic acid (D). In certain embodiments, the amino acid substitution at position 436 of SEQ ID NO: 14487 or SEQ ID NO: 14484 is a substitution of an isoleucine (I) for a valine (V). In certain embodiments, the amino acid substitution at position 456 of SEQ ID NO: 14487 or SEQ ID NO: 14484 is a substitution of a tyrosine (Y) for a methionine (M). In certain embodiments, the amino acid substitution at position 470 of SEQ ID NO: 14487 or SEQ ID NO: 14484 is a substitution of a phenylalanine (F) for a leucine (L). In certain embodiments, the amino acid substitution at position 485 of SEQ ID NO: 14487 or SEQ ID NO: 14484 is a substitution of a lysine (K) for a serine (S). In certain embodiments, the amino acid substitution at position 503 of SEQ ID NO: 14487 or SEQ ID NO: 14484 is a substitution of a leucine (L) for a methionine (M). In certain embodiments, the amino acid substitution at position 503 of SEQ ID NO: 14487 or SEQ ID NO: 14484 is a substitution of an isoleucine (I) for a methionine (M). In certain embodiments, the amino acid substitution at position 552 of SEQ ID NO: 14487 or SEQ ID NO: 14484 is a substitution of a lysine (K) for a valine (V). In certain embodiments, the amino acid substitution at position 570 of SEQ ID NO: 14487 or SEQ ID NO: 14484 is a substitution of a threonine (T) for an alanine (A). In certain embodiments, the amino acid substitution at position 591 of SEQ ID NO: 14487 or SEQ ID NO: 14484 is a substitution of a proline (P) for a glutamine (Q). In certain embodiments, the amino acid substitution at position 591 of SEQ ID NO: 14487 or SEQ ID NO: 14484 is a substitution of an arginine (R) for a glutamine (Q).

In certain embodiments of the methods of the disclosure, including those embodiments wherein the transposase comprises the above-described mutations at positions 30, 165, 282 and/or 538, the piggyBac™ transposase enzyme may comprise or the Super piggyBac™ transposase enzyme may further comprise an amino acid substitution at one or more of positions 103, 194, 372, 375, 450, 509 and 570 of the sequence of SEQ ID NO: 14487 or SEQ ID NO: 14484. In certain embodiments of the methods of the disclosure, including those embodiments wherein the transposase comprises the above-described mutations at positions 30, 165, 282 and/or 538, the piggyBac™ transposase enzyme may comprise or the Super piggyBac™ transposase enzyme may further comprise an amino acid substitution at two, three, four, five, six or more of positions 103, 194, 372, 375, 450, 509 and 570 of the sequence of SEQ ID NO: 14487 or SEQ ID NO: 14484. In certain embodiments, including those embodiments wherein the transposase comprises the above-described mutations at positions 30, 165, 282 and/or 538, the piggyBac™ transposase enzyme may comprise or the Super piggyBac™ transposase enzyme may further comprise an amino acid substitution at positions 103, 194, 372, 375, 450, 509 and 570 of the sequence of SEQ ID NO: 14487 or SEQ ID NO: 14484. In certain embodiments, the amino acid substitution at position 103 of SEQ ID NO: 14487 or SEQ ID NO: 14484 is a substitution of a proline (P) for a serine (S). In certain embodiments, the amino acid substitution at position 194 of SEQ ID NO: 14487 or SEQ ID NO: 14484 is a substitution of a valine (V) for a methionine (M). In certain embodiments, the amino acid substitution at position 372 of SEQ ID NO: 14487 or SEQ ID NO: 14484 is a substitution of an alanine (A) for an arginine (R). In certain embodiments, the amino acid substitution at position 375 of SEQ ID NO: 14487 or SEQ ID NO: 14484 is a substitution of an alanine (A) for a lysine (K). In certain embodiments, the amino acid substitution at position 450 of SEQ ID NO: 14487 or SEQ ID NO: 14484 is a substitution of an asparagine (N) for an aspartic acid (D). In certain embodiments, the amino acid substitution at position 509 of SEQ ID NO: 14487 or SEQ ID NO: 14484 is a substitution of a glycine (G) for a serine (S). In certain embodiments, the amino acid substitution at position 570 of SEQ ID NO: 14487 or SEQ ID NO: 14484 is a substitution of a serine (S) for an asparagine (N). In certain embodiments, the piggyBac™ transposase enzyme may comprise a substitution of a valine (V) for a methionine (M) at position 194 of SEQ ID NO: 14487. In certain embodiments, including those embodiments wherein the piggyBac™ transposase enzyme may comprise a substitution of a valine (V) for a methionine (M) at position 194 of SEQ ID NO: 14487, the piggyBac™ transposase enzyme may further comprise an amino acid substitution at positions 372, 375 and 450 of the sequence of SEQ ID NO: 14487 or SEQ ID NO: 14484. In certain embodiments, the piggyBac™ transposase enzyme may comprise a substitution of a valine (V) for a methionine (M) at position 194 of SEQ ID NO: 14487, a substitution of an alanine (A) for an arginine (R) at position 372 of SEQ ID NO: 14487, and a substitution of an alanine (A) for a lysine (K) at position 375 of SEQ ID NO: 14487. In certain embodiments, the piggyBac™ transposase enzyme may comprise a substitution of a valine (V) for a methionine (M) at position 194 of SEQ ID NO: 14487, a substitution of an alanine (A) for an arginine (R) at position 372 of SEQ ID NO: 14487, a substitution of an alanine (A) for a lysine (K) at position 375 of SEQ ID NO: 14487 and a substitution of an asparagine (N) for an aspartic acid (D) at position 450 of SEQ ID NO: 14487.

The disclosure provides a culture media for enhancing viability of a modified immune cell comprising IL-2, IL-21, IL-7, IL-15 or any combination thereof. The modified immune cell may be a T-lymphocyte, a Natural Killer (NK) cell, a Cytokine-induced Killer (CIK) cell or a Natural Killer T (NKT) cell. In some embodiments, the modified immune cell is a T-lymphocyte. In some embodiments, the T-lymphocyte is an early memory T-cell. In some embodiments, the T-lymphocyte is a stem cell-like T-cell. In some embodiments, the T-lymphocyte is a stem memory T cell (T_{SCM}). In some embodiments, the T-lymphocyte is a central memory T cell (T_{CM}). The modified immune cell may contain one or more exogenous DNA sequences. The modified immune cell may contain one or more exogenous RNA sequences. The modified immune cell may have been electroporated or nucleofected.

### BRIEF DESCRIPTION OF THE DRAWINGS

FIG. 1 is a series of graphs depicting transfection efficiency and cell viability following plasmid DNA nucleofection in primary human T lymphocytes.

FIG. 2 is a series of graphs depicting DNA cytotoxicity to T cells.

FIG. 3 is a series of graphs showing that DNA-mediated cytotoxicity in T cells is dose dependent.

FIG. 4 is a series of graphs showing that extracellular plasmid DNA is not cytotoxic.

FIG. 5 is a series of graphs depicting efficient transposition using SPB mRNA in Jurkat cells.

FIG. 6 is a series of graphs depicting efficient transposition in T lymphocytes using SPB mRNA.

FIG. 7 is a series of graphs depicting efficient delivery of linearized DNA transposon products.

FIG. 8 is a series of graphs showing that addition of that IL-7 and IL-15 and immediate stimulation of T cells post-nucleofection enhances cell viability.

FIG. 9 is a series of graphs showing that IL-7 and IL-15 rescue T cells from DNA mediated toxicity

FIG. 10 is a series of graphs showing that immediate stimulation of T cells post-nucleofection enhances cell viability.

FIG. 11A-C is a series of graphs depicting T cell transposition with varying amounts of DNA. Primary human pan T cells were nucleofected with varying amounts of DNA using piggyBac™. T cells were nucleofected with the indicated amounts of transposon and 5 μg SPB mRNA. Cells were then stimulated on day 2 post-nucleofection through CD3 and CD28. As expected, T cells nucleofected with high amounts of DNA exhibited high episomal expression at day 1 post nucleofection whereas almost no episomal expression was observed at low DNA doses. In contrast, following expansion at day 21 post nucleofection the greatest percentage of transgene positive cells were observed in lower DNA amounts peaking at 1.67 μg for this transposon. (A) Flow analysis for transgene positive cells at day 1 and 21. (B) Percentage of transgene positive T cells. (C) Percentage of viable T cells at day 1 and 21. For all graphs shown in this figure, the Y-axis ranges from 0 to 100% in increments of 20% and the X-axis ranges from 0 to 10⁵ by powers of 10.

FIG. 12A-B is a series of graphs depicting T cell transposition with low DNA amounts using the Sleeping Beauty™ 100X (SB100X) transposase. Primary human pan T cells were nucleofected with GFP plasmids encoding either the piggyBac™ (PB) or Sleeping Beauty™ (SB) ITRs. (A) Cells were nucleofected with the indicated amounts of SB transposon and 1 μg SB transposase mRNA. (B) Cells were nucleofected with the indicated amounts of SB transposase and 0.75 μg SB transposon. Flow analysis was performed on day 14 post nucleofection for all samples. For all graphs shown in this figure, the Y-axis ranges from 0 to 250K in increments of 50K and the X-axis ranges from 0 to 10⁵ by powers of 10.

FIG. 13A is a series of plots depicting T cells transposed with a plasmid containing a sequence encoding a transposon comprising a sequence encoding an inducible caspase polypeptide (a safety switch, “iC9”), a CARTyrin (anti-BCMA), and a selectable marker. Left-hand plots depict live T cells exposed to transposase in the absence of the plasmid. Right-hand plots depict live T cells exposed to transposase in the presence of the plasmid. Cells were exposed to either a hyperactive transposase (the “Super piggyBac”) or a wild type piggyBac transposase.

FIG. 13B is a series of plots depicting T cells transposed with a plasmid containing a sequence encoding a green fluorescent protein (GFP). Left-hand plots depict live T cells exposed to transposase in the absence of the plasmid. Right-hand plots depict live T cells exposed to transposase in the presence of the plasmid. Cells were exposed to either a hyperactive transposase (the “Super piggyBac”) or a wild type piggyBac transposase.

FIG. 13C is a table depicting the percent of transformed T cells resulting from transposition with WT versus hyperactive piggyBac transposase. T cells contacted with the hyperactive piggyBac transposase (the Super piggyBac transposase) were transformed at a rate 4-fold greater than WT transposase.

FIG. 13D is a graph depicting the percent of transformed T cells resulting from transposition with WT versus hyperactive piggyBac transposase 5 days after nucleofection. T cells contacted with the hyperactive piggyBac transposase (the Super piggyBac transposase) were transformed at a rate far greater than WT transposase.

FIG. 14 is a graph depicting transposition in natural killer (NK) cells. Transposition of non-activated NK cells derived from CD3-depleted leukopheresis (containing CD14/CD19/CD56+ cells) is shown. Cells were electroporated (EP) with plasmid piggyBac transposon DNA encoding GFP and mRNA encoding super piggyBac. The program from Lonza 4D nucleofector or BTX ECM 830 (500V, 700 usec pulse length, 0.2 mm electrode gap, one pulse) is indicated on the X-axis. Transposed cells were co-cultured (stimulated) at day 2 with artificial antigen presenting cells (aAPCs). Fluorescent activated cell sorting (FACS) analysis of percent GFP positive cells at day 7 post-EP (day 5 post-stim) is indicated on the Y-axis with gray bars. Percent viability as shown by percent 7-Aminoactinomycin D (7AAD)-negative cells at day 2 post-EP is indicated on the Y-axis with gray bars.

FIG. 15A-B are a series of 10 FACs plots (FIG. 15A) and a graph (FIG. 15B) showing transposon titration for transposition in natural killer (NK) cells. Transposition of non-activated NK cells from CD3-depleted leukopheresis (containing CD14/CD19/CD56+ cells) is shown. Cells were electroporated with a plasmid piggyBac transposon encoding GFP at amounts ranging from 0 to 10 ug of DNA and 5 ug mRNA encoding Super piggyBac using the indicated Maxcyte electroporator program. Transposed cells were stimulated at day 2 with artificial antigen presenting cells (aAPCs). FIG. 15A FACs plots top row shows CD56+(y-axis) versus GFP+(x-axis) expression, while the bottom row shows 7AAD (y-axis) versus forward scatter (FSC, x-axis). FIG. 15B is a bar graph analysis of the percentage of GFP+ cells of CD56+ cells at day 6 post-electroporation (EP) and day 4 post-stimulation (black bars), and the percent viability as shown by 7AAD-negative cells at day 2 post EP (gray bars).

FIG. 16A-B are a series of 7 FACs plots (FIG. 16A) and a graph (FIG. 16B) showing dose-dependent DNA-mediated cytotoxicity in NK cells. FACS analysis of live cells (7AAD-negative/FSC) at day 2 post-EP using the Lonza 4D Nucleofector program DN-100 are shown (FIG. 16A). FACS plots (FIG. 16A) are quantified in a graph (FIG. 16B). 5E6 cells per EP were electroporated in 100 uL P3 buffer in cuvettes. Cells were electroporated with no DNA (Mock) or varying amounts of piggyBac GFP transposon co-delivered with 5 ug Super piggyBac mRNA.

FIG. 17 is a series of 5 graphs showing the in vitro differentiation of piggyBac modified hematopoietic stem and precursor cells (HSPCs) into B cells. Human CD34+ HSPCs were electroporated with mRNA encoding Super piggyBac along with a piggyBac transposon encoding GFP. After electroporation, HSPCs were primed for B cell differentiation in presence of human IL-3, Flt3L, TPO, SCF, and G-CSF for 5 days. On day 6, cells were transferred to a layer of MS-5 feeder cells and fed bi-weekly, along with transfer to a fresh layer of feeders once per week. On day 34 of the in vitro differentiation process, CD19+ B cells were generated and detectable in the culture. Top row: FACs plots showing CD19 (y-axis) and CD34 (x-axis) in, from left to right, human primary bone marrow cells, at day 6 of in vitro differentiation, and at day 34 of in vitro differentiation. Bottom row: graphs depicting GFP expression in the indicated boxed populations of cells from the FACs plots in the top row at days 6 and 34 of in vitro differentiation.

FIG. 18 is a schematic depiction of the Csy4-T2A-Clo051-G4Slinker-dCas9 construct map.

FIG. 19 is a schematic depiction of the pRT1-Clo051-dCas9 Double NLS construct map.

### DETAILED DESCRIPTION

Disclosed are compositions and methods for the ex-vivo genetic modification of an immune cell or a precursor thereof comprising delivering to the immune cell or immune precursor cell, (a) a nucleic acid or amino acid sequence comprising a sequence encoding a transposase enzyme and (b) a recombinant and non-naturally occurring DNA sequence comprising a DNA sequence encoding a transposon. In certain embodiments, the method further comprises the step of stimulating the immune cell or immune precursor cell with one or more cytokine(s).

### Immune and Immune Precursor Cells

In certain embodiments, immune cells of the disclosure comprise lymphoid progenitor cells, natural killer (NK) cells, T lymphocytes (T-cell), stem memory T cells (T_{SCM} cells), Stem cell-like T cells, B lymphocytes (B-cells), myeloid progenitor cells, neutrophils, basophils, eosinophils, monocytes, macrophages, platelets, erythrocytes, red blood cells (RBCs), megakaryocytes or osteoclasts.

In certain embodiments, immune precursor cells comprise any cells which can differentiate into one or more types of immune cells. In certain embodiments, immune precursor cells comprise multipotent stem cells that can self renew and develop into immune cells. In certain embodiments, immune precursor cells comprise hematopoietic stem cells (HSCs) or descendants thereof. In certain embodiments, immune precursor cells comprise precursor cells that can develop into immune cells. In certain embodiments, the immune precursor cells comprise hematopoietic progenitor cells (HPCs).

### Hematopoietic Stem Cells (HSCs)

Hematopoietic stem cells (HSCs) are multipotent, self-renewing cells. All differentiated blood cells from the lymphoid and myeloid lineages arise from HSCs. HSCs can be found in adult bone marrow, peripheral blood, mobilized peripheral blood, peritoneal dialysis effluent and umbilical cord blood.

HSCs of the disclosure may be isolated or derived from a primary or cultured stem cell. HSCs of the disclosure may be isolated or derived from an embryonic stem cell, a multipotent stem cell, a pluripotent stem cell, an adult stem cell, or an induced pluripotent stem cell (iPSC).

Immune precursor cells of the disclosure may comprise an HSC or an HSC descendent cell. Exemplary HSC descendent cells of the disclosure include, but are not limited to, multipotent stem cells, lymphoid progenitor cells, natural killer (NK) cells, T lymphocyte cells (T-cells), B lymphocyte cells (B-cells), myeloid progenitor cells, neutrophils, basophils, eosinophils, monocytes, and macrophages.

HSCs produced by the methods of the disclosure may retain features of “primitive” stem cells that, while isolated or derived from an adult stem cell and while committed to a single lineage, share characteristics of embryonic stem cells. For example, the “primitive” HSCs produced by the methods of the disclosure retain their “stemness” following division and do not differentiate. Consequently, as an adoptive cell therapy, the “primitive” HSCs produced by the methods of the disclosure not only replenish their numbers, but expand in vivo. “Primitive” HSCs produced by the methods of the disclosure may be therapeutically-effective when administered as a single dose. In some embodiments, primitive HSCs of the disclosure are CD34+. In some embodiments, primitive HSCs of the disclosure are CD34+ and CD38−. In some embodiments, primitive HSCs of the disclosure are CD34+, CD38− and CD90+. In some embodiments, primitive HSCs of the disclosure are CD34+, CD38−, CD90+ and CD45RA−. In some embodiments, primitive HSCs of the disclosure are CD34+, CD38−, CD90+, CD45RA−, and CD49f+. In some embodiments, the most primitive HSCs of the disclosure are CD34+, CD38−, CD90+, CD45RA−, and CD49f+.

In some embodiments of the disclosure, primitive HSCs, HSCs, and/or HSC descendent cells may be modified according to the methods of the disclosure to express an exogenous sequence (e.g. a chimeric antigen receptor or therapeutic protein). In some embodiments of the disclosure, modified primitive HSCs, modified HSCs, and/or modified HSC descendent cells may be forward differentiated to produce a modified immune cell including, but not limited to, a modified T cell, a modified natural killer cell and/or a modified B-cell of the disclosure.

### T Cells

Modified T cells of the disclosure may be derived from modified hematopoietic stem and progenitor cells (HSPCs) or modified HSCs.

Unlike traditional biologics and chemotherapeutics, modified-T cells of the disclosure possess the capacity to rapidly reproduce upon antigen recognition, thereby potentially obviating the need for repeat treatments. To achieve this, in some embodiments, modified-T cells of the disclosure not only drive an initial response, but also persist in the patient as a stable population of viable memory T cells to prevent potential relapses. Alternatively, in some embodiments, when it is not desired, modified-T cells of the disclosure do not persist in the patient.

Intensive efforts have been focused on the development of antigen receptor molecules that do not cause T cell exhaustion through antigen-independent (tonic) signaling, as well as of a modified-T cell product containing early memory T cells, especially stem cell memory (T_{SCM}) or stem cell-like T cells. Stem cell-like modified-T cells of the disclosure exhibit the greatest capacity for self-renewal and multipotent capacity to derive central memory (T_{CM}) T cells or T_{CM} like cells, effector memory (T_{EM}) and effector T cells (T_{E}), thereby producing better tumor eradication and long-term modified-T cell engraftment. A linear pathway of differentiation may be responsible for generating these cells: Naïve T cells (T_{N})>T_{SCM}>T_{CM}>T_{EM}>T_{E}>T_{TE}, whereby T_{N} is the parent precursor cell that directly gives rise to T_{SCM}, which then, in turn, directly gives rise to T_{CM}, etc. Compositions of T cells of the disclosure may comprise one or more of each parental T cell subset with T_{SCM} cells being the most abundant (e.g. T_{SCM}>T_{CM}>T_{EM}>T_{E}>T_{TE}).

In some embodiments of the methods of the disclosure, the immune cell precursor is differentiated into or is capable of differentiating into an early memory T cell, a stem cell like T-cell, a Naïve T cells (T_{N}), a T_{SCM}, a T_{CM}, a T_{EM}, a T_{E}, or a T_{TE}. In some embodiments, the immune cell precursor is a primitive HSC, an HSC, or a HSC descendent cell of the disclosure.

In some embodiments of the methods of the disclosure, the immune cell is an early memory T cell, a stem cell like T-cell, a Naïve T cells (T_{N}), a T_{SCM}, a T_{CM}, a T_{EM}, a T_{E}, or a T_{TE}.

In some embodiments of the methods of the disclosure, the immune cell is an early memory T cell.

In some embodiments of the methods of the disclosure, the immune cell is a stem cell like T-cell.

In some embodiments of the methods of the disclosure, the immune cell is a T_{SCM}.

In some embodiments of the methods of the disclosure, the immune cell is a T_{CM}.

In some embodiments of the methods of the disclosure, the methods modify and/or the methods produce a plurality of modified T cells, wherein at least 2%, 5%, 10%, 15%, 20%, 25%, 30%, 35%, 40%, 45%, 50%, 60%, 65%, 70%, 75%, 80%, 85%, 90%, 95%, 99% or any percentage in between of the plurality of modified T cells expresses one or more cell-surface marker(s) of an early memory T cell. In certain embodiments, the plurality of modified early memory T cells comprises at least one modified stem cell-like T cell. In certain embodiments, the plurality of modified early memory T cells comprises at least one modified T_{SCM}. In certain embodiments, the plurality of modified early memory T cells comprises at least one modified T_{CM}.

In some embodiments of the methods of the disclosure, the methods modify and/or the methods produce a plurality of modified T cells, wherein at least 2%, 5%, 10%, 15%, 20%, 25%, 30%, 35%, 40%, 45%, 50%, 60%, 65%, 70%, 75%, 80%, 85%, 90%, 95%, 99% or any percentage in between of the plurality of modified T cells expresses one or more cell-surface marker(s) of a stem cell-like T cell. In certain embodiments, the plurality of modified stem cell-like T cells comprises at least one modified T_{SCM}. In certain embodiments, the plurality of modified stem cell-like T cells comprises at least one modified T_{CM}.

In some embodiments of the methods of the disclosure, the methods modify and/or the methods produce a plurality of modified T cells, wherein at least 2%, 5%, 10%, 15%, 20%, 25%, 30%, 35%, 40%, 45%, 50%, 60%, 65%, 70%, 75%, 80%, 85%, 90%, 95%, 99% or any percentage in between of the plurality of modified T cells expresses one or more cell-surface marker(s) of a stem memory T cell (T_{SCM}). In certain embodiments, the cell-surface markers comprise CD62L and CD45RA. In certain embodiments, the cell-surface markers comprise one or more of CD62L, CD45RA, CD28, CCR7, CD127, CD45RO, CD95, CD95 and IL-2Rβ. In certain embodiments, the cell-surface markers comprise one or more of CD45RA, CD95, CCR7, and CD62L.

In some embodiments of the methods of the disclosure, the methods modify and/or the methods produce a plurality of modified T cells, wherein at least 2%, 5%, 10%, 15%, 20%, 25%, 30%, 35%, 40%, 45%, 50%, 60%, 65%, 70%, 75%, 80%, 85%, 90%, 95%, 99% or any percentage in between of the plurality of modified T cells expresses one or more cell-surface marker(s) of a central memory T cell (T_{CM}). In certain embodiments, the cell-surface markers comprise one or more of CD45RO, CD95, CCR7, and CD62L.

In some embodiments of the methods of the disclosure, the methods modify and/or the methods produce a plurality of modified T cells, wherein at least 2%, 5%, 10%, 15%, 20%, 25%, 30%, 35%, 40%, 45%, 50%, 60%, 65%, 70%, 75%, 80%, 85%, 90%, 95%, 99% or any percentage in between of the plurality of modified T cells expresses one or more cell-surface marker(s) of a naïve T cell (T_{N}). In certain embodiments, the cell-surface markers comprise one or more of CD45RA, CCR7 and CD62L.

In some embodiments of the methods of the disclosure, the methods modify and/or the methods produce a plurality of modified T cells, wherein at least 2%, 5%, 10%, 15%, 20%, 25%, 30%, 35%, 40%, 45%, 50%, 60%, 65%, 70%, 75%, 80%, 85%, 90%, 95%, 99% or any percentage in between of the plurality of modified T cells expresses one or more cell-surface marker(s) of an effector T-cell (modified T_{EFF}). In certain embodiments, the cell-surface markers comprise one or more of CD45RA, CD95, and IL-2Rβ.

In some embodiments of the methods of the disclosure, the methods modify and/or the methods produce a plurality of modified T cells, wherein at least 2%, 5%, 10%, 15%, 20%, 25%, 30%, 35%, 40%, 45%, 50%, 60%, 65%, 70%, 75%, 80%, 85%, 90%, 95%, 99% or any percentage in between of the plurality of modified T cells expresses one or more cell-surface marker(s) of a stem cell-like T cell, a stem memory T cell (T_{SCM}) or a central memory T cell (T_{CM}).

In some embodiments of the methods of the disclosure, a buffer comprises the immune cell or precursor thereof. The buffer maintains or enhances a level of cell viability and/or a stem-like phenotype of the immune cell or precursor thereof, including T-cells. In certain embodiments, the buffer maintains or enhances a level of cell viability and/or a stem-like phenotype of the primary human T cells prior to the nucleofection. In certain embodiments, the buffer maintains or enhances a level of cell viability and/or a stem-like phenotype of the primary human T cells during the nucleofection. In certain embodiments, the buffer maintains or enhances a level of cell viability and/or a stem-like phenotype of the primary human T cells following the nucleofection. In certain embodiments, the buffer comprises one or more of KCl, MgCl₂, ClNa, Glucose and Ca (NO₃)₂ in any absolute or relative abundance or concentration, and, optionally, the buffer further comprises a supplement selected from the group consisting of HEPES, Tris/HCl, and a phosphate buffer. In certain embodiments, the buffer comprises 5 mM KCl, 15 mM MgCl₂, 90 mM ClNa, 10 mM Glucose and 0.4 mM Ca(NO₃)₂. In certain embodiments, the buffer comprises 5 mM KCl, 15 mM MgCl₂, 90 mM ClNa, 10 mM Glucose and 0.4 mM Ca(NO₃)₂ and a supplement comprising 20 mM HEPES and 75 mM Tris/HCl. In certain embodiments, the buffer comprises 5 mM KCl, 15 mM MgCl₂, 90 mM ClNa, 10 mM Glucose and 0.4 mM Ca(NO₃)₂ and a supplement comprising 40 mM Na₂HPO₄/NaH₂PO₄ at pH 7.2. In certain embodiments, the composition comprising primary human T cells comprises 100 μl of the buffer and between 5×10⁶ and 25×10⁶ cells. In certain embodiments, the composition comprises a scalable ratio of 250e6 primary human T cells per milliliter of buffer or other media during the introduction step.

In some embodiments of the methods of the disclosure, the introducing step may comprise delivery of transposon and/or transposase by a method other than electroporation or nucleofection. In some embodiments, a composition comprises a scalable ratio of 250e6 primary human T cells per milliliter of buffer or other media during the introduction step.

In some embodiments of the methods of the disclosure, the introducing step comprises one or more of topical delivery, adsorption, absorption, electroporation, spin-fection, co-culture, transfection, mechanical delivery, sonic delivery, vibrational delivery, magnetofection or by nanoparticle-mediated delivery.

In some embodiments of the methods of the disclosure, the introducing step comprises liposomal transfection, calcium phosphate transfection, fugene transfection, and dendrimer-mediated transfection.

In some embodiments of the methods of the disclosure, the introducing step comprises mechanical transfection comprises cell squeezing, cell bombardment, or gene gun techniques.

In some embodiments of the methods of the disclosure, the introducing step comprises nanoparticle-mediated transfection comprises liposomal delivery, delivery by micelles, and delivery by polymerosomes.

In some embodiments of the methods of the disclosure, the methods comprise contacting an immune cell of the disclosure, including a T cell of the disclosure, and a T-cell expansion composition. In some embodiments of the methods of the disclosure, the step of introducing a transposon and/or transposase of the disclosure into an immune cell of the disclosure may further comprise contacting the immune cell and a T-cell expansion composition. In some embodiments, including those in which the introducing step of the methods comprises an electroporation or a nucleofection step, the electroporation or a nucleofection step may be performed with the immune cell contacting T-cell expansion composition of the disclosure.

In some embodiments of the methods of the disclosure, the T-cell expansion composition comprises, consists essentially of or consists of phosphorus; one or more of an octanoic acid, a palmitic acid, a linoleic acid, and an oleic acid; a sterol; and an alkane.

In certain embodiments of the methods of producing a modified T cell of the disclosure, the expansion supplement comprises one or more cytokine(s). The one or more cytokine(s) may comprise any cytokine, including but not limited to, lymphokines. Exemplary lympokines include, but are not limited to, interleukin-2 (IL-2), interleukin-3 (IL-3), interleukin-4 (IL-4), interleukin-5 (IL-5), interleukin-6 (IL-6), interleukin-7 (IL-7), interleukin-15 (IL-15), interleukin-21 (IL-21), granulocyte-macrophage colony-stimulating factor (GM-CSF) and interferon-gamma (INFγ). The one or more cytokine(s) may comprise IL-2.

In some embodiments of the methods of the disclosure, the T-cell expansion composition comprises human serum albumin, recombinant human insulin, human transferrin, 2-Mercaptoethanol, and an expansion supplement. In certain embodiments of this method, the T-cell expansion composition further comprises one or more of octanoic acid, nicotinamide, 2,4,7,9-tetramethyl-5-decyn-4,7-diol (TMDD), diisopropyl adipate (DIPA), n-butyl-benzenesulfonamide, 1,2-benzenedicarboxylic acid, bis(2-methylpropyl) ester, palmitic acid, linoleic acid, oleic acid, stearic acid hydrazide, oleamide, a sterol and an alkane. In certain embodiments of this method, the T-cell expansion composition further comprises one or more of octanoic acid, palmitic acid, linoleic acid, oleic acid and a sterol. In certain embodiments of this method, the T-cell expansion composition further comprises one or more of octanoic acid at a concentration of between 0.9 mg/kg to 90 mg/kg, inclusive of the endpoints; palmitic acid at a concentration of between 0.2 mg/kg to 20 mg/kg, inclusive of the endpoints; linoleic acid at a concentration of between 0.2 mg/kg to 20 mg/kg, inclusive of the endpoints; oleic acid at a concentration of 0.2 mg/kg to 20 mg/kg, inclusive of the endpoints; and a sterol at a concentration of about 0.1 mg/kg to 10 mg/kg, inclusive of the endpoints. In certain embodiments of this method, the T-cell expansion composition further comprises one or more of octanoic acid at a concentration of about 9 mg/kg, palmitic acid at a concentration of about 2 mg/kg, linoleic acid at a concentration of about 2 mg/kg, oleic acid at a concentration of about 2 mg/kg and a sterol at a concentration of about 1 mg/kg. In certain embodiments of this method, the T-cell expansion composition further comprises one or more of octanoic acid at a concentration of between 6.4 μmol/kg and 640 μmol/kg, inclusive of the endpoints; palmitic acid at a concentration of between 0.7 μmol/kg and 70 μmol/kg, inclusive of the endpoints; linoleic acid at a concentration of between 0.75 μmol/kg and 75 μmol/kg, inclusive of the endpoints; oleic acid at a concentration of between 0.75 μmol/kg and 75 μmol/kg, inclusive of the endpoints; and a sterol at a concentration of between 0.25 μmol/kg and 25 μmol/kg, inclusive of the endpoints. In certain embodiments of this method, the T-cell expansion composition further comprises one or more of octanoic acid at a concentration of about 64 μmol/kg, palmitic acid at a concentration of about 7 μmol/kg, linoleic acid at a concentration of about 7.5 μmol/kg, oleic acid at a concentration of about 7.5 μmol/kg and a sterol at a concentration of about 2.5 μmol/kg.

In certain embodiments, the T-cell expansion composition comprises one or more of human serum albumin, recombinant human insulin, human transferrin, 2-Mercaptoethanol, and an expansion supplement to produce a plurality of expanded modified T-cells, wherein at least 2% of the plurality of modified T-cells expresses one or more cell-surface marker(s) of an early memory T cell, a stem cell-like T cell, a stem memory T cell (T_{SCM}) and/or a central memory T cell (T_{CM}). In certain embodiments, the T-cell expansion composition comprises or further comprises one or more of octanoic acid, nicotinamide, 2,4,7,9-tetramethyl-5-decyn-4,7-diol (TMDD), diisopropyl adipate (DIPA), n-butyl-benzenesulfonamide, 1,2-benzenedicarboxylic acid, bis(2-methylpropyl) ester, palmitic acid, linoleic acid, oleic acid, stearic acid hydrazide, oleamide, a sterol and an alkane. In certain embodiments, the T-cell expansion composition comprises one or more of octanoic acid, palmitic acid, linoleic acid, oleic acid and a sterol (e.g. cholesterol). In certain embodiments, the T-cell expansion composition comprises one or more of octanoic acid at a concentration of between 0.9 mg/kg to 90 mg/kg, inclusive of the endpoints; palmitic acid at a concentration of between 0.2 mg/kg to 20 mg/kg, inclusive of the endpoints; linoleic acid at a concentration of between 0.2 mg/kg to 20 mg/kg, inclusive of the endpoints; oleic acid at a concentration of 0.2 mg/kg to 20 mg/kg, inclusive of the endpoints; and a sterol at a concentration of about 0.1 mg/kg to 10 mg/kg, inclusive of the endpoints (wherein mg/kg=parts per million). In certain embodiments, the T-cell expansion composition comprises one or more of octanoic acid at a concentration of about 9 mg/kg, palmitic acid at a concentration of about 2 mg/kg, linoleic acid at a concentration of about 2 mg/kg, oleic acid at a concentration of about 2 mg/kg, and a sterol at a concentration of about 1 mg/kg (wherein mg/kg=parts per million). In certain embodiments, the T-cell expansion composition comprises one or more of octanoic acid at a concentration of 9.19 mg/kg, palmitic acid at a concentration of 1.86 mg/kg, linoleic acid at a concentration of about 2.12 mg/kg, oleic acid at a concentration of about 2.13 mg/kg, and a sterol at a concentration of about 1.01 mg/kg (wherein mg/kg=parts per million). In certain embodiments, the T-cell expansion composition comprises octanoic acid at a concentration of 9.19 mg/kg, palmitic acid at a concentration of 1.86 mg/kg, linoleic acid at a concentration of 2.12 mg/kg, oleic acid at a concentration of about 2.13 mg/kg, and a sterol at a concentration of 1.01 mg/kg (wherein mg/kg=parts per million). In certain embodiments, the T-cell expansion composition comprises one or more of octanoic acid at a concentration of between 6.4 μmol/kg and 640 μmol/kg, inclusive of the endpoints; palmitic acid at a concentration of between 0.7 μmol/kg and 70 μmol/kg, inclusive of the endpoints; linoleic acid at a concentration of between 0.75 μmol/kg and 75 μmol/kg, inclusive of the endpoints; oleic acid at a concentration of between 0.75 μmol/kg and 75 μmol/kg, inclusive of the endpoints; and a sterol at a concentration of between 0.25 μmol/kg and 25 μmol/kg, inclusive of the endpoints. In certain embodiments, the T-cell expansion composition comprises one or more of octanoic acid at a concentration of about 64 μmol/kg, palmitic acid at a concentration of about 7 μmol/kg, linoleic acid at a concentration of about 7.5 μmol/kg, oleic acid at a concentration of about 7.5 μmol/kg and a sterol at a concentration of about 2.5 μmol/kg. In certain embodiments, the T-cell expansion composition comprises one or more of octanoic acid at a concentration of about 63.75 μmol/kg, palmitic acid at a concentration of about 7.27 μmol/kg, linoleic acid at a concentration of about 7.57 μmol/kg, oleic acid at a concentration of about 7.56 μmol/kg and a sterol at a concentration of about 2.61 μmol/kg. In certain embodiments, the T-cell expansion composition comprises octanoic acid at a concentration of about 63.75 μmol/kg, palmitic acid at a concentration of about 7.27 μmol/kg, linoleic acid at a concentration of about 7.57 μmol/kg, oleic acid at a concentration of 7.56 μmol/kg and a sterol at a concentration of 2.61 μmol/kg.

As used herein, the terms “supplemented T-cell expansion composition” or “T-cell expansion composition” may be used interchangeably with a media comprising one or more of human serum albumin, recombinant human insulin, human transferrin, 2-Mercaptoethanol, and an expansion supplement at 37° C. Alternatively, or in addition, the terms “supplemented T-cell expansion composition” or “T-cell expansion composition” may be used interchangeably with a media comprising one or more of phosphorus, an octanoic fatty acid, a palmitic fatty acid, a linoleic fatty acid and an oleic acid. In certain embodiments, the media comprises an amount of phosphorus that is 10-fold higher than may be found in, for example, Iscove's Modified Dulbecco's Medium ((IMDM); available at ThermoFisher Scientific as Catalog number 12440053).

As used herein, the terms “supplemented T-cell expansion composition” or “T-cell expansion composition” may be used interchangeably with a media comprising one or more of human serum albumin, recombinant human insulin, human transferrin, 2-Mercaptoethanol, Iscove's MDM, and an expansion supplement at 37° C. Alternatively, or in addition, the terms “supplemented T-cell expansion composition” or “T-cell expansion composition” may be used interchangeably with a media comprising one or more of the following elements: boron, sodium, magnesium, phosphorus, potassium, and calcium. In certain embodiments, the terms “supplemented T-cell expansion composition” or “T-cell expansion composition” may be used interchangeably with a media comprising one or more of the following elements present in the corresponding average concentrations: boron at 3.7 mg/L, sodium at 3000 mg/L, magnesium at 18 mg/L, phosphorus at 29 mg/L, potassium at 15 mg/L and calcium at 4 mg/L.

As used herein, the terms “supplemented T-cell expansion composition” or “T-cell expansion composition” may be used interchangeably with a media comprising one or more of human serum albumin, recombinant human insulin, human transferrin, 2-Mercaptoethanol, and an expansion supplement at 37° C. Alternatively, or in addition, the terms “supplemented T-cell expansion composition” or “T-cell expansion composition” may be used interchangeably with a media comprising one or more of the following components: octanoic acid (CAS No. 124-07-2), nicotinamide (CAS No. 98-92-0), 2,4,7,9-tetramethyl-5-decyn-4,7-diol (TMDD) (CAS No. 126-86-3), diisopropyl adipate (DIPA) (CAS No. 6938-94-9), n-butyl-benzenesulfonamide (CAS No. 3622-84-2), 1,2-benzenedicarboxylic acid, bis(2-methylpropyl) ester (CAS No. 84-69-5), palmitic acid (CAS No. 57-10-3), linoleic acid (CAS No. 60-33-3), oleic acid (CAS No. 112-80-1), stearic acid hydrazide (CAS No. 4130-54-5), oleamide (CAS No. 3322-62-1), sterol (e.g., cholesterol) (CAS No. 57-88-5), and alkanes (e.g., nonadecane) (CAS No. 629-92-5). In certain embodiments, the terms “supplemented T-cell expansion composition” or “T-cell expansion composition” may be used interchangeably with a media comprising one or more of the following components: octanoic acid (CAS No. 124-07-2), nicotinamide (CAS No. 98-92-0), 2,4,7,9-tetramethyl-5-decyn-4,7-diol (TMDD) (CAS No. 126-86-3), diisopropyl adipate (DIPA) (CAS No. 6938-94-9), n-butyl-benzenesulfonamide (CAS No. 3622-84-2), 1,2-benzenedicarboxylic acid, bis(2-methylpropyl) ester (CAS No. 84-69-5), palmitic acid (CAS No. 57-10-3), linoleic acid (CAS No. 60-33-3), oleic acid (CAS No. 112-80-1), stearic acid hydrazide (CAS No. 4130-54-5), oleamide (CAS No. 3322-62-1), sterol (e.g., cholesterol) (CAS No. 57-88-5), alkanes (e.g., nonadecane) (CAS No. 629-92-5), and phenol red (CAS No. 143-74-8). In certain embodiments, the terms “supplemented T-cell expansion composition” or “T-cell expansion composition” may be used interchangeably with a media comprising one or more of the following components: octanoic acid (CAS No. 124-07-2), nicotinamide (CAS No. 98-92-0), 2,4,7,9-tetramethyl-5-decyn-4,7-diol (TMDD) (CAS No. 126-86-3), diisopropyl adipate (DIPA) (CAS No. 6938-94-9), n-butyl-benzenesulfonamide (CAS No. 3622-84-2), 1,2-benzenedicarboxylic acid, bis(2-methylpropyl) ester (CAS No. 84-69-5), palmitic acid (CAS No. 57-10-3), linoleic acid (CAS No. 60-33-3), oleic acid (CAS No. 112-80-1), stearic acid hydrazide (CAS No. 4130-54-5), oleamide (CAS No. 3322-62-1), phenol red (CAS No. 143-74-8) and lanolin alcohol.

In certain embodiments, the terms “supplemented T-cell expansion composition” or “T-cell expansion composition” may be used interchangeably with a media comprising one or more of human serum albumin, recombinant human insulin, human transferrin, 2-Mercaptoethanol, and an expansion supplement at 37° C. Alternatively, or in addition, the terms “supplemented T-cell expansion composition” or “T-cell expansion composition” may be used interchangeably with a media comprising one or more of the following ions: sodium, ammonium, potassium, magnesium, calcium, chloride, sulfate and phosphate.

As used herein, the terms “supplemented T-cell expansion composition” or “T-cell expansion composition” may be used interchangeably with a media comprising one or more of human serum albumin, recombinant human insulin, human transferrin, 2-Mercaptoethanol, and an expansion supplement at 37° C. Alternatively, or in addition, the terms “supplemented T-cell expansion composition” or “T-cell expansion composition” may be used interchangeably with a media comprising one or more of the following free amino acids: histidine, asparagine, serine, glutamate, arginine, glycine, aspartic acid, glutamic acid, threonine, alanine, proline, cysteine, lysine, tyrosine, methionine, valine, isoleucine, leucine, phenylalanine and tryptophan. In certain embodiments, the terms “supplemented T-cell expansion composition” or “T-cell expansion composition” may be used interchangeably with a media comprising one or more of the following free amino acids in the corresponding average mole percentages: histidine (about 1%), asparagine (about 0.5%), serine (about 1.5%), glutamine (about 67%), arginine (about 1.5%), glycine (about 1.5%), aspartic acid (about 1%), glutamic acid (about 2%), threonine (about 2%), alanine (about 1%), proline (about 1.5%), cysteine (about 1.5%), lysine (about 3%), tyrosine (about 1.5%), methionine (about 1%), valine (about 3.5%), isoleucine (about 3%), leucine (about 3.5%), phenylalanine (about 1.5%) and tryptophan (about 0.5%). In certain embodiments, the terms “supplemented T-cell expansion composition” or “T-cell expansion composition” may be used interchangeably with a media comprising one or more of the following free amino acids in the corresponding average mole percentages: histidine (about 0.78%), asparagine (about 0.4%), serine (about 1.6%), glutamine (about 67.01%), arginine (about 1.67%), glycine (about 1.72%), aspartic acid (about 1.00%), glutamic acid (about 1.93%), threonine (about 2.38%), alanine (about 1.11%), proline (about 1.49%), cysteine (about 1.65%), lysine (about 2.84%), tyrosine (about 1.62%), methionine (about 0.85%), valine (about 3.45%), isoleucine (about 3.14%), leucine (about 3.3%), phenylalanine (about 1.64%) and tryptophan (about 0.37%).

As used herein, the terms “supplemented T-cell expansion composition” or “T-cell expansion composition” may be used interchangeably with a media comprising one or more of human serum albumin, recombinant human insulin, human transferrin, 2-Mercaptoethanol, Iscove's MDM, and an expansion supplement at 37° C. Alternatively, or in addition, the terms “supplemented T-cell expansion composition” or “T-cell expansion composition” may be used interchangeably with a media comprising one or more of phosphorus, an octanoic fatty acid, a palmitic fatty acid, a linoleic fatty acid and an oleic acid. In certain embodiments, the media comprises an amount of phosphorus that is 10-fold higher than may be found in, for example, Iscove's Modified Dulbecco's Medium ((IMDM); available at ThermoFisher Scientific as Catalog number 12440053).

In certain embodiments, the terms “supplemented T-cell expansion composition” or “T-cell expansion composition” may be used interchangeably with a media comprising one or more of octanoic acid, palmitic acid, linoleic acid, oleic acid and a sterol (e.g. cholesterol). In certain embodiments, the terms “supplemented T-cell expansion composition” or “T-cell expansion composition” may be used interchangeably with a media comprising one or more of octanoic acid at a concentration of between 0.9 mg/kg to 90 mg/kg, inclusive of the endpoints; palmitic acid at a concentration of between 0.2 mg/kg to 20 mg/kg, inclusive of the endpoints; linoleic acid at a concentration of between 0.2 mg/kg to 20 mg/kg, inclusive of the endpoints; oleic acid at a concentration of 0.2 mg/kg to 20 mg/kg, inclusive of the endpoints; and a sterol at a concentration of about 0.1 mg/kg to 10 mg/kg, inclusive of the endpoints (wherein mg/kg=parts per million). In certain embodiments, the terms “supplemented T-cell expansion composition” or “T-cell expansion composition” may be used interchangeably with a media comprising one or more of octanoic acid at a concentration of about 9 mg/kg, palmitic acid at a concentration of about 2 mg/kg, linoleic acid at a concentration of about 2 mg/kg, oleic acid at a concentration of about 2 mg/kg, and a sterol at a concentration of about 1 mg/kg (wherein mg/kg=parts per million). In certain embodiments, the terms “supplemented T-cell expansion composition” or “T-cell expansion composition” may be used interchangeably with a media comprising one or more of octanoic acid at a concentration of 9.19 mg/kg, palmitic acid at a concentration of 1.86 mg/kg, linoleic acid at a concentration of about 2.12 mg/kg, oleic acid at a concentration of about 2.13 mg/kg, and a sterol at a concentration of about 1.01 mg/kg (wherein mg/kg=parts per million). In certain embodiments, the terms “supplemented T-cell expansion composition” or “T-cell expansion composition” may be used interchangeably with a media comprising one or more of octanoic acid at a concentration of 9.19 mg/kg, palmitic acid at a concentration of 1.86 mg/kg, linoleic acid at a concentration of 2.12 mg/kg, oleic acid at a concentration of about 2.13 mg/kg, and a sterol at a concentration of 1.01 mg/kg (wherein mg/kg=parts per million). In certain embodiments, the terms “supplemented T-cell expansion composition” or “T-cell expansion composition” may be used interchangeably with a media comprising one or more of octanoic acid at a concentration of between 6.4 μmol/kg and 640 μmol/kg, inclusive of the endpoints; palmitic acid at a concentration of between 0.7 μmol/kg and 70 μmol/kg, inclusive of the endpoints; linoleic acid at a concentration of between 0.75 μmol/kg and 75 μmol/kg, inclusive of the endpoints; oleic acid at a concentration of between 0.75 μmol/kg and 75 μmol/kg, inclusive of the endpoints; and a sterol at a concentration of between 0.25 μmol/kg and 25 μmol/kg, inclusive of the endpoints. In certain embodiments, the terms “supplemented T-cell expansion composition” or “T-cell expansion composition” may be used interchangeably with a media comprising one or more of octanoic acid at a concentration of about 64 μmol/kg, palmitic acid at a concentration of about 7 μmol/kg, linoleic acid at a concentration of about 7.5 μmol/kg, oleic acid at a concentration of about 7.5 μmol/kg and a sterol at a concentration of about 2.5 μmol/kg.

In certain embodiments, the terms “supplemented T-cell expansion composition” or “T-cell expansion composition” may be used interchangeably with a media comprising one or more of octanoic acid at a concentration of about 63.75 μmol/kg, palmitic acid at a concentration of about 7.27 μmol/kg, linoleic acid at a concentration of about 7.57 μmol/kg, oleic acid at a concentration of about 7.56 μmol/kg and a sterol at a concentration of about 2.61 μmol/kg. In certain embodiments, the terms “supplemented T-cell expansion composition” or “T-cell expansion composition” may be used interchangeably with a media comprising one or more of octanoic acid at a concentration of about 63.75 μmol/kg, palmitic acid at a concentration of about 7.27 μmol/kg, linoleic acid at a concentration of about 7.57 μmol/kg, oleic acid at a concentration of 7.56 μmol/kg and a sterol at a concentration of 2.61 μmol/kg.

Modified T-cells of the disclosure, including modified stem cell-like T cells, T_{SCM} and/or T_{CM} of the disclosure, may be incubated, cultured, grown, stored, or otherwise, combined at any step in the methods of the procedure with a growth medium comprising one or more inhibitors a component of a PI3K pathway. Exemplary inhibitors a component of a PI3K pathway include, but are not limited to, an inhibitor of GSK3β such as TWS119 (also known as GSK 3B inhibitor XII; CAS Number 601514-19-6 having a chemical formula C₁₈H₁₄N₄O₂). Exemplary inhibitors of a component of a PI3K pathway include, but are not limited to, bb007 (BLUEBIRDBIO™).

In some embodiments of the methods of the disclosure, the methods comprise contacting an immune cell of the disclosure and a T-cell activator composition. In some embodiments of the methods of the disclosure, the methods comprise contacting an immune cell precursor of the disclosure and a T-cell activator composition. In some embodiments of the methods of the disclosure, the methods comprise contacting a modified T cell of the disclosure and a T-cell activator composition. In some embodiments, the T-cell activator composition comprises one or more of an anti-human CD3 monospecific tetrameric antibody complex, an anti-human CD28 monospecific tetrameric antibody complex and an activation supplement to produce an activated modified T-cell or a plurality of activated modified T-cells. In some embodiments, the activated modified T-cell expresses one or more cell-surface marker(s) of an early memory T cell, a stem cell-like T cell, a T_{SCM} or a T_{CM}. In some embodiments, at least 2%, 5%, 10%, 15%, 20%, 25%, 30%, 35%, 40%, 45%, 50%, 60%, 65%, 70%, 75%, 80%, 85%, 90%, 95%, 99% or any percentage in between of the plurality of activated modified T-cells express one or more cell-surface marker(s) of an early memory T cell, a stem cell-like T cell, a T_{SCM} or a T_{CM}.

In certain embodiments of the methods of producing a modified T cell (e.g. a stem cell-like T cell, a T_{SCM} and/or a T_{CM}) of the disclosure, the activation supplement may comprise one or more cytokine(s). The one or more cytokine(s) may comprise any cytokine, including but not limited to, lymphokines. Exemplary lympokines include, but are not limited to, interleukin-2 (IL-2), interleukin-3 (IL-3), interleukin-4 (IL-4), interleukin-5 (IL-5), interleukin-6 (IL-6), interleukin-7 (IL-7), interleukin-15 (IL-15), interleukin-21 (IL-21), granulocyte-macrophage colony-stimulating factor (GM-CSF) and interferon-gamma (INFγ). The one or more cytokine(s) may comprise IL-2.

### Natural Killer (NK) Cells

In certain embodiments, the modified immune or immune precursor cells of the disclosure are natural killer (NK) cells. In certain embodiments, NK cells are cytotoxic lymphocytes that differentiate from lymphoid progenitor cells.

Modified NK cells of the disclosure may be derived from modified hematopoietic stem and progenitor cells (HSPCs) or modified HSCs.

In certain embodiments, non-activated NK cells are derived from CD3-depleted leukopheresis (containing CD14/CD19/CD56+ cells).

In certain embodiments, NK cells are electroporated using a Lonza 4D nucleofector or BTX ECM 830 (500V, 700 usec pulse length, 0.2 mm electrode gap, one pulse). All Lonza 4D nucleofector programs are contemplated as within the scope of the methods of the disclosure.

In certain embodiments, 5×10E6 cells were electroporated per electroporation in 100 μL P3 buffer in cuvettes. However, this ratio of cells per volume is scalable for commercial manufacturing methods.

In certain embodiments, NK cells were stimulated by co-culture with an additional cell line. In certain embodiments, the additional cell line comprises artificial antigen presenting cells (aAPCs). In certain embodiments, stimulation occurs at day 1, 2, 3, 4, 5, 6, or 7 following electroporation. In certain embodiments, stimulation occurs at day 2 following electroporation.

In certain embodiments, NK cells express CD56.

B cells

In certain embodiments, the modified immune or immune precursor cells of the disclosure are B cells. B cells are a type of lymphocyte that express B cell receptors on the cell surface. B cell receptors bind to specific antigens.

Modified B cells of the disclosure may be derived from modified hematopoietic stem and progenitor cells (HSPCs) or modified HSCs.

In certain embodiments, HSPCs are modified using the methods of the disclosure, and then primed for B cell differentiation in presence of human IL-3, Flt3L, TPO, SCF, and G-CSF for at least 3 days, at least 4 days, at least 5 days, at least 6 days or at least 7 days. In certain embodiments, HSPCs are modified using the methods of the disclosure, and then primed for B cell differentiation in presence of human IL-3, Flt3L, TPO, SCF, and G-CSF for 5 days.

In certain embodiments, following priming, modified HSPC cells are transferred to a layer of feeder cells and fed bi-weekly, along with transfer to a fresh layer of feeders once per week. In certain embodiments, the feeder cells are MS-5 feeder cells.

In certain embodiments, modified HSPC cells are cultured with MS-5 feeder cells for at least 7, 14, 21, 28, 30, 33, 35, 42 or 48 days. In certain embodiments, modified HSPC cells were cultured with MS-5 feeder cells for 33 days.

### Chimeric Antigen Receptors

In certain embodiments, a modified immune or pre-immune cell of the disclosure comprises a chimeric antigen receptor.

In certain embodiments of the methods of the disclosure, the recombinant and non-naturally occurring DNA sequence encoding a transposon further comprises a sequence encoding a chimeric antigen receptor or a portion thereof. Chimeric antigen receptors (CARs) of the disclosure may comprise (a) an ectodomain comprising an antigen recognition region, (b) a transmembrane domain, and (c) an endodomain comprising at least one costimulatory domain. In certain embodiments, the ectodomain may further comprise a signal peptide. Alternatively, or in addition, in certain embodiments, the ectodomain may further comprise a hinge between the antigen recognition region and the transmembrane domain. In certain embodiments of the CARs of the disclosure, the signal peptide may comprise a sequence encoding a human CD2, CD3δ, CD3ε, CD3γ, CD3ζ, CD4, CD8α, CD19, CD28, 4-1BB or GM-CSFR signal peptide. In certain embodiments of the CARs of the disclosure, the signal peptide may comprise a sequence encoding a human CD8α signal peptide. In certain embodiments, the transmembrane domain may comprise a sequence encoding a human CD2, CD3δ, CD3ε, CD3γ, CD3ζ, CD4, CD8α, CD19, CD28, 4-1BB or GM-CSFR transmembrane domain. In certain embodiments of the CARs of the disclosure, the transmembrane domain may comprise a sequence encoding a human CD8α transmembrane domain. In certain embodiments of the CARs of the disclosure, the endodomain may comprise a human CD3 endodomain.

In certain embodiments of the CARs of the disclosure, the at least one costimulatory domain may comprise a human 4-1BB, CD28, CD40, ICOS, MyD88, OX-40 intracellular segment, or any combination thereof. In certain embodiments of the CARs of the disclosure, the at least one costimulatory domain may comprise a CD28 and/or a 4-1BB costimulatory domain. In certain embodiments of the CARs of the disclosure, the hinge may comprise a sequence derived from a human CD8α, IgG4, and/or CD4 sequence. In certain embodiments of the CARs of the disclosure, the hinge may comprise a sequence derived from a human CD8α sequence.

The CD28 costimulatory domain may comprise an amino acid sequence comprising

(SEQ ID NO: 14659)
RVKFSRSADAPAYKQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

or a sequence having at least 70%, 80%, 90%, 95%, or 99% identity to the amino acid sequence comprising

(SEQ ID NO: 14659)
RVKFSRSADAPAYKQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR.

The CD28 costimulatory domain may be encoded by the nucleic acid sequence comprising

(SEQ ID NO: 14660)
cgcgtgaagtttagtcgatcagcagatgccccagcttacaaacagggaca
gaaccagctgtataacgagctgaatctgggccgccgagaggaatatgacg
tgctggataagcggagaggacgcgaccccgaaatgggaggcaagcccagg
cgcaaaaaccctcaggaaggcctgtataacgagctgcagaaggacaaaat
ggcagaagcctattctgagatcggcatgaagggggagcgacggagaggca
aagggcacgatgggctgtaccagggactgagcaccgccacaaaggacacc
tatgatgctctgcatatgcaggcactgcctccaagg.

The 4-1BB costimulatory domain may comprise an amino acid sequence comprising

(SEQ ID NO: 14661)
KRGRKKLLYIFKQPFMRPVQTTQEEDGCSCRFPEEEEGGCEL

or a sequence having at least 70%, 80%, 90%, 95%, or 99% identity to the amino acid sequence comprising

(SEQ ID NO: 14661)
KRGRKKLLYIFKQPFMRPVQTTQEEDGCSCRFPEEEEGGCEL.

The 4-1BB costimulatory domain may be encoded by the nucleic acid sequence comprising

(SEQ ID NO: 14662)
aagagaggcaggaagaaactgctgtatattttcaaacagcccttcatgcg
ccccgtgcagactacccaggaggaagacgggtgctcctgtcgattccctg
aggaagaggaaggcgggtgtgagctg.

The 4-1BB costimulatory domain may be located between the transmembrane domain and the CD28 costimulatory domain.

In certain embodiments of the CARs of the disclosure, the hinge may comprise a sequence derived from a human CD8α, IgG4, and/or CD4 sequence. In certain embodiments of the CARs of the disclosure, the hinge may comprise a sequence derived from a human CD8α sequence. The hinge may comprise a human CD8α amino acid sequence comprising

(SEQ ID NO: 14663)
TTTPAPRPPTPAPTIASQPLSLRPEACRPAAGGAVHTRGLDFACD

or a sequence having at least 70%, 80%, 90%, 95%, or 99% identity to the amino acid sequence comprising

(SEQ ID NO: 14663)
TTTPAPRPPTPAPTIASQPLSLRPEACRPAAGGAVHTRGLDFACD.

The human CD8α hinge amino acid sequence may be encoded by the nucleic acid sequence comprising

(SEQ ID NO: 14664)
actaccacaccagcacctagaccaccaactccagctccaaccatcgcgag
tcagcccctgagtctgagacctgaggcctgcaggccagctgcaggaggag
ctgtgcacaccaggggcctggacttcgcctgcgac.

### ScFv

The disclosure provides single chain variable fragment (scFv) compositions and methods for use of these compositions to recognize and bind to a specific target protein. ScFv compositions comprise a heavy chain variable region and a light chain variable region of an antibody. ScFv compositions may be incorporated into an antigen recognition region of a chimeric antigen receptor of the disclosure. ScFvs are fusion proteins of the variable regions of the heavy (VH) and light (VL) chains of immunoglobulins, and the VH and VL domains are connected with a short peptide linker. ScFvs retain the specificity of the original immunoglobulin, despite removal of the constant regions and the introduction of the linker. An exemplary linker comprises a sequence of GGGGSGGGGSGGGGS (SEQ ID NO: 14665).

### Centyrins

Centyrins of the disclosure specifically bind to an antigen. Chimeric antigen receptors of the disclosure comprising one or more Centyrins that specifically bind an antigen may be used to direct the specificity of a cell, (e.g. a cytotoxic immune cell) towards the specific antigen.

Centyrins of the disclosure may comprise a protein scaffold, wherein the scaffold is capable of specifically binding an antigen. Centyrins of the disclosure may comprise a protein scaffold comprising a consensus sequence of at least one fibronectin type III (FN3) domain, wherein the scaffold is capable of specifically binding an antigen. The at least one fibronectin type III (FN3) domain may be derived from a human protein. The human protein may be Tenascin-C. The consensus sequence may comprise

(SEQ ID NO: 14488)
LPAPKNLVVSEVTEDSLRLSWTAPDAAFDSFLIQYQESEKVGEAINLTVP
GSERSYDLTGLKPGTEYTVSIYGVKGGHRSNPLSAEFTT
or
MLPAPKNLVVSEVTEDSLRLSWTAPDAAFDSFLIQYQESEKVGEAINLTV
PGSERSYD

The consensus sequence may comprise an amino sequence at least 74% identical to

(SEQ ID NO: 14488)
LPAPKNLVVSEVTEDSLRLSWTAPDAAFDSFLIQYQESEKVGEAINLTVP
GSERSYDLTGLKPGTEYTVSIYGVKGGHRSNPLSAEFTT
or
(SEQ ID NO: 14489)
MLPAPKNLVVSEVTEDSLRLSWTAPDAAFDSFLIQYQESEKVGEAINLTV
PGSERSYDLTGLKPGTEYTVSIYGVKGGHRSNPLSAEFTT.

The consensus sequence may encoded by a nucleic acid sequence comprising

(SEQ ID NO: 14490)
atgctgcctgcaccaaagaacctggtggtgtctcatgtgacagaggatag
tgccagactgtcatggactgctcccgacgcagccttcgatagttttatca
tcgtgtaccgggagaacatcgaaaccggcgaggccattgtcctgacagtg
ccagggtccgaacgctcttatgacctgacagatctgaagcccggaactga
gtactatgtgcagatcgccggcgtcaaaggaggcaatatcagcttccctc
tgtccgcaatcttcaccaca.

The consensus sequence may be modified at one or more positions within (a) a A-B loop comprising or consisting of the amino acid residues TEDS (SEQ ID NO: 14491) at positions 13-16 of the consensus sequence; (b) a B-C loop comprising or consisting of the amino acid residues TAPDAAF (SEQ ID NO: 14492) at positions 22-28 of the consensus sequence; (c) a C-D loop comprising or consisting of the amino acid residues SEKVGE (SEQ ID NO: 14493) at positions 38-43 of the consensus sequence; (d) a D-E loop comprising or consisting of the amino acid residues GSER (SEQ ID NO: 14494) at positions 51-54 of the consensus sequence; (e) a E-F loop comprising or consisting of the amino acid residues GLKPG (SEQ ID NO: 14495) at positions 60-64 of the consensus sequence; (f) a F-G loop comprising or consisting of the amino acid residues KGGHRSN (SEQ ID NO: 14496) at positions 75-81 of the consensus sequence; or (g) any combination of (a)-(f). Centyrins of the disclosure may comprise a consensus sequence of at least 5 fibronectin type III (FN3) domains, at least 10 fibronectin type III (FN3) domains or at least 15 fibronectin type III (FN3) domains. The scaffold may bind an antigen with at least one affinity selected from a K_{D} of less than or equal to 10M, less than or equal to 10⁻¹⁰ M, less than or equal to 10⁻¹¹ M, less than or equal to 10⁻¹²M, less than or equal to 10⁻¹³M, less than or equal to 10⁻¹⁴M, and less than or equal to 10⁻¹⁵M. The K_{D} may be determined by surface plasmon resonance.

The term “antibody mimetic” is intended to describe an organic compound that specifically binds a target sequence and has a structure distinct from a naturally-occurring antibody. Antibody mimetics may comprise a protein, a nucleic acid, or a small molecule. The target sequence to which an antibody mimetic of the disclosure specifically binds may be an antigen. Antibody mimetics may provide superior properties over antibodies including, but not limited to, superior solubility, tissue penetration, stability towards heat and enzymes (e.g. resistance to enzymatic degradation), and lower production costs. Exemplary antibody mimetics include, but are not limited to, an affibody, an afflilin, an affimer, an affitin, an alphabody, an anticalin, and avimer (also known as avidity multimer), a DARPin (Designed Ankyrin Repeat Protein), a Fynomer, a Kunitz domain peptide, and a monobody.

Affibody molecules of the disclosure comprise a protein scaffold comprising or consisting of one or more alpha helix without any disulfide bridges. Preferably, affibody molecules of the disclosure comprise or consist of three alpha helices. For example, an affibody molecule of the disclosure may comprise an immunoglobulin binding domain. An affibody molecule of the disclosure may comprise the Z domain of protein A.

Affilin molecules of the disclosure comprise a protein scaffold produced by modification of exposed amino acids of, for example, either gamma-B crystallin or ubiquitin. Affilin molecules functionally mimic an antibody's affinity to antigen, but do not structurally mimic an antibody. In any protein scaffold used to make an affilin, those amino acids that are accessible to solvent or possible binding partners in a properly-folded protein molecule are considered exposed amino acids. Any one or more of these exposed amino acids may be modified to specifically bind to a target sequence or antigen.

Affimer molecules of the disclosure comprise a protein scaffold comprising a highly stable protein engineered to display peptide loops that provide a high affinity binding site for a specific target sequence. Exemplary affimer molecules of the disclosure comprise a protein scaffold based upon a cystatin protein or tertiary structure thereof. Exemplary affimer molecules of the disclosure may share a common tertiary structure of comprising an alpha-helix lying on top of an anti-parallel beta-sheet.

Affitin molecules of the disclosure comprise an artificial protein scaffold, the structure of which may be derived, for example, from a DNA binding protein (e.g. the DNA binding protein Sac7d). Affitins of the disclosure selectively bind a target sequence, which may be the entirety or part of an antigen. Exemplary affitins of the disclosure are manufactured by randomizing one or more amino acid sequences on the binding surface of a DNA binding protein and subjecting the resultant protein to ribosome display and selection. Target sequences of affitins of the disclosure may be found, for example, in the genome or on the surface of a peptide, protein, virus, or bacteria. In certain embodiments of the disclosure, an affitin molecule may be used as a specific inhibitor of an enzyme. Affitin molecules of the disclosure may include heat-resistant proteins or derivatives thereof.

Alphabody molecules of the disclosure may also be referred to as Cell-Penetrating Alphabodies (CPAB). Alphabody molecules of the disclosure comprise small proteins (typically of less than 10 kDa) that bind to a variety of target sequences (including antigens). Alphabody molecules are capable of reaching and binding to intracellular target sequences. Structurally, alphabody molecules of the disclosure comprise an artificial sequence forming single chain alpha helix (similar to naturally occurring coiled-coil structures). Alphabody molecules of the disclosure may comprise a protein scaffold comprising one or more amino acids that are modified to specifically bind target proteins. Regardless of the binding specificity of the molecule, alphabody molecules of the disclosure maintain correct folding and thermostability.

Anticalin molecules of the disclosure comprise artificial proteins that bind to target sequences or sites in either proteins or small molecules. Anticalin molecules of the disclosure may comprise an artificial protein derived from a human lipocalin. Anticalin molecules of the disclosure may be used in place of, for example, monoclonal antibodies or fragments thereof. Anticalin molecules may demonstrate superior tissue penetration and thermostability than monoclonal antibodies or fragments thereof. Exemplary anticalin molecules of the disclosure may comprise about 180 amino acids, having a mass of approximately 20 kDa. Structurally, anticalin molecules of the disclosure comprise a barrel structure comprising antiparallel beta-strands pairwise connected by loops and an attached alpha helix. In preferred embodiments, anticalin molecules of the disclosure comprise a barrel structure comprising eight antiparallel beta-strands pairwise connected by loops and an attached alpha helix.

Avimer molecules of the disclosure comprise an artificial protein that specifically binds to a target sequence (which may also be an antigen). Avimers of the disclosure may recognize multiple binding sites within the same target or within distinct targets. When an avimer of the disclosure recognize more than one target, the avimer mimics function of a bi-specific antibody. The artificial protein avimer may comprise two or more peptide sequences of approximately 30-35 amino acids each. These peptides may be connected via one or more linker peptides. Amino acid sequences of one or more of the peptides of the avimer may be derived from an A domain of a membrane receptor. Avimers have a rigid structure that may optionally comprise disulfide bonds and/or calcium. Avimers of the disclosure may demonstrate greater heat stability compared to an antibody.

DARPins (Designed Ankyrin Repeat Proteins) of the disclosure comprise genetically-engineered, recombinant, or chimeric proteins having high specificity and high affinity for a target sequence. In certain embodiments, DARPins of the disclosure are derived from ankyrin proteins and, optionally, comprise at least three repeat motifs (also referred to as repetitive structural units) of the ankyrin protein. Ankyrin proteins mediate high-affinity protein-protein interactions. DARPins of the disclosure comprise a large target interaction surface.

Fynomers of the disclosure comprise small binding proteins (about 7 kDa) derived from the human Fyn SH3 domain and engineered to bind to target sequences and molecules with equal affinity and equal specificity as an antibody.

Kunitz domain peptides of the disclosure comprise a protein scaffold comprising a Kunitz domain. Kunitz domains comprise an active site for inhibiting protease activity. Structurally, Kunitz domains of the disclosure comprise a disulfide-rich alpha+beta fold. This structure is exemplified by the bovine pancreatic trypsin inhibitor. Kunitz domain peptides recognize specific protein structures and serve as competitive protease inhibitors. Kunitz domains of the disclosure may comprise Ecallantide (derived from a human lipoprotein-associated coagulation inhibitor (LACI)).

Monobodies of the disclosure are small proteins (comprising about 94 amino acids and having a mass of about 10 kDa) comparable in size to a single chain antibody. These genetically engineered proteins specifically bind target sequences including antigens. Monobodies of the disclosure may specifically target one or more distinct proteins or target sequences. In preferred embodiments, monobodies of the disclosure comprise a protein scaffold mimicking the structure of human fibronectin, and more preferably, mimicking the structure of the tenth extracellular type III domain of fibronectin. The tenth extracellular type III domain of fibronectin, as well as a monobody mimetic thereof, contains seven beta sheets forming a barrel and three exposed loops on each side corresponding to the three complementarity determining regions (CDRs) of an antibody. In contrast to the structure of the variable domain of an antibody, a monobody lacks any binding site for metal ions as well as a central disulfide bond. Multispecific monobodies may be optimized by modifying the loops BC and FG. Monobodies of the disclosure may comprise an adnectin.

### VHH

In certain embodiments, the CAR comprises a single domain antibody (SdAb). In certain embodiments, the SdAb is a VHH.

The disclosure provides chimeric antigen receptors (CARs) comprising at least one VHH (a VCAR). Chimeric antigen receptors of the disclosure may comprise more than one VHH. For example, a bi-specific VCAR may comprise two VHHs that specifically bind two distinct antigens.

VHH proteins of the disclosure specifically bind to an antigen. Chimeric antigen receptors of the disclosure comprising one or more VHHs that specifically bind an antigen may be used to direct the specificity of a cell, (e.g. a cytotoxic immune cell) towards the specific antigen.

At least one VHH protein or VCAR of the disclosure can be optionally produced by a cell line, a mixed cell line, an immortalized cell or clonal population of immortalized cells, as well known in the art. See, e.g., Ausubel, et al., ed., Current Protocols in Molecular Biology, John Wiley & Sons, Inc., NY, N.Y. (1987-2001); Sambrook, et al., Molecular Cloning: A Laboratory Manual, 2nd Edition, Cold Spring Harbor, N.Y. (1989); Harlow and Lane, Antibodies, a Laboratory Manual, Cold Spring Harbor, N.Y. (1989); Colligan, et al., eds., Current Protocols in Immunology, John Wiley & Sons, Inc., NY (1994-2001); Colligan et al., Current Protocols in Protein Science, John Wiley & Sons, NY, N.Y., (1997-2001).

Amino acids from a VHH protein can be altered, added and/or deleted to reduce immunogenicity or reduce, enhance or modify binding, affinity, on-rate, off-rate, avidity, specificity, half-life, stability, solubility or any other suitable characteristic, as known in the art.

Optionally, VHH proteins can be engineered with retention of high affinity for the antigen and other favorable biological properties. To achieve this goal, the VHH proteins can be optionally prepared by a process of analysis of the parental sequences and various conceptual engineered products using three-dimensional models of the parental and engineered sequences. Three-dimensional models are commonly available and are familiar to those skilled in the art. Computer programs are available which illustrate and display probable three-dimensional conformational structures of selected candidate sequences and can measure possible immunogenicity (e.g., Immunofilter program of Xencor, Inc. of Monrovia, Calif.). Inspection of these displays permits analysis of the likely role of the residues in the functioning of the candidate sequence, i.e., the analysis of residues that influence the ability of the candidate VHH protein to bind its antigen. In this way, residues can be selected and combined from the parent and reference sequences so that the desired characteristic, such as affinity for the target antigen(s), is achieved. Alternatively, or in addition to, the above procedures, other suitable methods of engineering can be used.

Screening VHH for specific binding to similar proteins or fragments can be conveniently achieved using nucleotide (DNA or RNA display) or peptide display libraries, for example, in vitro display. This method involves the screening of large collections of peptides for individual members having the desired function or structure. The displayed nucleotide or peptide sequences can be from 3 to 5000 or more nucleotides or amino acids in length, frequently from 5-100 amino acids long, and often from about 8 to 25 amino acids long. In addition to direct chemical synthetic methods for generating peptide libraries, several recombinant DNA methods have been described. One type involves the display of a peptide sequence on the surface of a bacteriophage or cell. Each bacteriophage or cell contains the nucleotide sequence encoding the particular displayed peptide sequence. The VHH proteins of the disclosure can bind human or other mammalian proteins with a wide range of affinities (KD). In a preferred embodiment, at least one VHH of the present invention can optionally bind to a target protein with high affinity, for example, with a KD equal to or less than about 10⁻⁷ M, such as but not limited to, 0.1-9.9 (or any range or value therein)×10⁻⁸, 10⁻⁹, 10⁻¹⁰, 10⁻¹¹, 10⁻¹², 10⁻¹³, 10⁻¹⁴, 10⁻¹⁵ or any range or value therein, as determined by surface plasmon resonance or the Kinexa method, as practiced by those of skill in the art.

The affinity or avidity of a VHH or a VCAR for an antigen can be determined experimentally using any suitable method. (See, for example, Berzofsky, et al., “Antibody-Antigen Interactions,” In Fundamental Immunology, Paul, W. E., Ed., Raven Press: New York, N.Y. (1984); Kuby, Janis Immunology, W.H. Freeman and Company: New York, N.Y. (1992); and methods described herein). The measured affinity of a particular VHH-antigen or VCAR-antigen interaction can vary if measured under different conditions (e.g., salt concentration, pH). Thus, measurements of affinity and other antigen-binding parameters (e.g., KD, Kon, Koff) are preferably made with standardized solutions of VHH or VCAR and antigen, and a standardized buffer, such as the buffer described herein.

Competitive assays can be performed with the VHH or VCAR of the disclosure in order to determine what proteins, antibodies, and other antagonists compete for binding to a target protein with the VHH or VCAR of the present invention and/or share the epitope region. These assays as readily known to those of ordinary skill in the art evaluate competition between antagonists or ligands for a limited number of binding sites on a protein. The protein and/or antibody is immobilized or insolubilized before or after the competition and the sample bound to the target protein is separated from the unbound sample, for example, by decanting (where the protein/antibody was preinsolubilized) or by centrifuging (where the protein/antibody was precipitated after the competitive reaction). Also, the competitive binding may be determined by whether function is altered by the binding or lack of binding of the VHH or VCAR to the target protein, e.g., whether the VCAR molecule inhibits or potentiates the enzymatic activity of, for example, a label. ELISA and other functional assays may be used, as well known in the art.

### VH

In certain embodiments, the CAR comprises a single domain antibody (SdAb). In certain embodiments, the SdAb is a VH.

The disclosure provides chimeric antigen receptors (CARs) comprising a single domain antibody (VCARs). In certain embodiments, the single domain antibody comprises a VH. In certain embodiments, the VH is isolated or derived from a human sequence. In certain embodiments, VH comprises a human CDR sequence and/or a human framework sequence and a non-human or humanized sequence (e.g. a rat Fc domain). In certain embodiments, the VH is a fully humanized VH. In certain embodiments, the VH s neither a naturally occurring antibody nor a fragment of a naturally occurring antibody. In certain embodiments, the VH is not a fragment of a monoclonal antibody. In certain embodiments, the VH is a UniDab™ antibody (TeneoBio).

In certain embodiments, the VH is fully engineered using the UniRat™ (TeneoBio) system and “NGS-based Discovery” to produce the VH. Using this method, the specific VH are not naturally-occurring and are generated using fully engineered systems. The VH are not derived from naturally-occurring monoclonal antibodies (mAbs) that were either isolated directly from the host (for example, a mouse, rat or human) or directly from a single clone of cells or cell line (hybridoma). These VHs were not subsequently cloned from said cell lines. Instead, VH sequences are fully-engineered using the UniRat™ system as transgenes that comprise human variable regions (VH domains) with a rat Fc domain, and are thus human/rat chimeras without a light chain and are unlike the standard mAb format. The native rat genes are knocked out and the only antibodies expressed in the rat are from transgenes with VH domains linked to a Rat Fc (UniAbs). These are the exclusive Abs expressed in the UniRat. Next generation sequencing (NGS) and bioinformatics are used to identify the full antigen-specific repertoire of the heavy-chain antibodies generated by UniRat™ after immunization. Then, a unique gene assembly method is used to convert the antibody repertoire sequence information into large collections of fully-human heavy-chain antibodies that can be screened in vitro for a variety of functions. In certain embodiments, fully humanized VH are generated by fusing the human VH domains with human Fcs in vitro (to generate a non-naturally occurring recombinant VH antibody). In certain embodiments, the VH are fully humanized, but they are expressed in vivo as human/rat chimera (human VH, rat Fc) without a light chain. Fully humanized VHs are expressed in vivo as human/rat chimera (human VH, rat Fc) without a light chain are about 80 kDa (vs 150 kDa).

VCARs of the disclosure may comprise at least one VH of the disclosure. In certain embodiments, the VH of the disclosure may be modified to remove an Fc domain or a portion thereof. In certain embodiments, a framework sequence of the VH of the disclosure may be modified to, for example, improve expression, decrease immunogenicity or to improve function.

As used throughout the disclosure, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more standard deviations. Alternatively, “about” can mean a range of up to 20%, or up to 10%, or up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The disclosure provides isolated or substantially purified polynucleotide or protein compositions. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the protein of the invention or biologically active portion thereof is recombinantly produced, optimally culture medium represents less than about 30%, 20%, 10%, 5%, or 1% (by dry weight) of chemical precursors or non-protein-of-interest chemicals.

The disclosure provides fragments and variants of the disclosed DNA sequences and proteins encoded by these DNA sequences. As used throughout the disclosure, the term “fragment” refers to a portion of the DNA sequence or a portion of the amino acid sequence and hence protein encoded thereby. Fragments of a DNA sequence comprising coding sequences may encode protein fragments that retain biological activity of the native protein and hence DNA recognition or binding activity to a target DNA sequence as herein described. Alternatively, fragments of a DNA sequence that are useful as hybridization probes generally do not encode proteins that retain biological activity or do not retain promoter activity. Thus, fragments of a DNA sequence may range from at least about 20 nucleotides, about 50 nucleotides, about 100 nucleotides, and up to the full-length polynucleotide of the invention.

Nucleic acids or proteins of the disclosure can be constructed by a modular approach including preassembling monomer units and/or repeat units in target vectors that can subsequently be assembled into a final destination vector. Polypeptides of the disclosure may comprise repeat monomers of the disclosure and can be constructed by a modular approach by preassembling repeat units in target vectors that can subsequently be assembled into a final destination vector. The disclosure provides polypeptide produced by this method as well nucleic acid sequences encoding these polypeptides. The disclosure provides host organisms and cells comprising nucleic acid sequences encoding polypeptides produced this modular approach.

The term “antibody” is used in the broadest sense and specifically covers single monoclonal antibodies (including agonist and antagonist antibodies) and antibody compositions with polyepitopic specificity. It is also within the scope hereof to use natural or synthetic analogs, mutants, variants, alleles, homologs and orthologs (herein collectively referred to as “analogs”) of the antibodies hereof as defined herein. Thus, according to one embodiment hereof, the term “antibody hereof” in its broadest sense also covers such analogs. Generally, in such analogs, one or more amino acid residues may have been replaced, deleted and/or added, compared to the antibodies hereof as defined herein.

“Antibody fragment”, and all grammatical variants thereof, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (l) single-chain Fv (scFv) molecules (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific or multivalent structures formed from antibody fragments. In an antibody fragment comprising one or more heavy chains, the heavy chain(s) can contain any constant domain sequence (e.g. CHI in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s). The term further includes single domain antibodies (“sdAB”) which generally refers to an antibody fragment having a single monomeric variable antibody domain, (for example, from camelids). Such antibody fragment types will be readily understood by a person having ordinary skill in the art.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific.

The term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of shall mean excluding more than trace elements of other ingredients and substantial method steps. Embodiments defined by each of these transition terms are within the scope of this invention.

The term “epitope” refers to an antigenic determinant of a polypeptide. An epitope could comprise three amino acids in a spatial conformation, which is unique to the epitope. Generally, an epitope consists of at least 4, 5, 6, or 7 such amino acids, and more usually, consists of at least 8, 9, or 10 such amino acids. Methods of determining the spatial conformation of amino acids are known in the art, and include, for example, x-ray crystallography and two-dimensional nuclear magnetic resonance.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

“Gene expression” refers to the conversion of the information, contained in a gene, into a gene product. A gene product can be the direct transcriptional product of a gene (e.g., mRNA, tRNA, rRNA, antisense RNA, ribozyme, shRNA, micro RNA, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” or “regulation” of gene expression refers to a change in the activity of a gene. Modulation of expression can include, but is not limited to, gene activation and gene repression.

The term “operatively linked” or its equivalents (e.g., “linked operatively”) means two or more molecules are positioned with respect to each other such that they are capable of interacting to affect a function attributable to one or both molecules or a combination thereof.

Non-covalently linked components and methods of making and using non-covalently linked components, are disclosed. The various components may take a variety of different forms as described herein. For example, non-covalently linked (i.e., operatively linked) proteins may be used to allow temporary interactions that avoid one or more problems in the art. The ability of non-covalently linked components, such as proteins, to associate and dissociate enables a functional association only or primarily under circumstances where such association is needed for the desired activity. The linkage may be of duration sufficient to allow the desired effect.

A method for directing proteins to a specific locus in a genome of an organism is disclosed. The method may comprise the steps of providing a DNA localization component and providing an effector molecule, wherein the DNA localization component and the effector molecule are capable of operatively linking via a non-covalent linkage.

The term “scFv” refers to a single-chain variable fragment. scFv is a fusion protein of the variable regions of the heavy (VH) and light chains (VL) of immunoglobulins, connected with a linker peptide. The linker peptide may be from about 5 to 40 amino acids or from about 10 to 30 amino acids or about 5, 10, 15, 20, 25, 30, 35, or 40 amino acids in length. Single-chain variable fragments lack the constant Fc region found in complete antibody molecules, and, thus, the common binding sites (e.g., Protein G) used to purify antibodies. The term further includes a scFv that is an intrabody, an antibody that is stable in the cytoplasm of the cell, and which may bind to an intracellular protein.

The term “single domain antibody” means an antibody fragment having a single monomeric variable antibody domain which is able to bind selectively to a specific antigen. A single-domain antibody generally is a peptide chain of about 110 amino acids long, comprising one variable domain (VH) of a heavy-chain antibody, or of a common IgG, which generally have similar affinity to antigens as whole antibodies, but are more heat-resistant and stable towards detergents and high concentrations of urea. Examples are those derived from camelid or fish antibodies. Alternatively, single-domain antibodies can be made from common murine or human IgG with four chains.

The terms “specifically bind” and “specific binding” as used herein refer to the ability of an antibody, an antibody fragment or a nanobody to preferentially bind to a particular antigen that is present in a homogeneous mixture of different antigens. In certain embodiments, a specific binding interaction will discriminate between desirable and undesirable antigens in a sample. In certain embodiments more than about ten- to 100-fold or more (e.g., more than about 1000- or 10,000-fold). “Specificity” refers to the ability of an immunoglobulin or an immunoglobulin fragment, such as a nanobody, to bind preferentially to one antigenic target versus a different antigenic target and does not necessarily imply high affinity.

A “target site” or “target sequence” is a nucleic acid sequence that defines a portion of a nucleic acid to which a binding molecule will bind, provided sufficient conditions for binding exist.

The terms “nucleic acid” or “oligonucleotide” or “polynucleotide” refer to at least two nucleotides covalently linked together. The depiction of a single strand also defines the sequence of the complementary strand. Thus, a nucleic acid may also encompass the complementary strand of a depicted single strand. A nucleic acid of the disclosure also encompasses substantially identical nucleic acids and complements thereof that retain the same structure or encode for the same protein.

Probes of the disclosure may comprise a single stranded nucleic acid that can hybridize to a target sequence under stringent hybridization conditions. Thus, nucleic acids of the disclosure may refer to a probe that hybridizes under stringent hybridization conditions.

Nucleic acids of the disclosure may be single- or double-stranded. Nucleic acids of the disclosure may contain double-stranded sequences even when the majority of the molecule is single-stranded. Nucleic acids of the disclosure may contain single-stranded sequences even when the majority of the molecule is double-stranded. Nucleic acids of the disclosure may include genomic DNA, cDNA, RNA, or a hybrid thereof. Nucleic acids of the disclosure may contain combinations of deoxyribo- and ribo-nucleotides. Nucleic acids of the disclosure may contain combinations of bases including uracil, adenine, thymine, cytosine, guanine, inosine, xanthine hypoxanthine, isocytosine and isoguanine. Nucleic acids of the disclosure may be synthesized to comprise non-natural amino acid modifications. Nucleic acids of the disclosure may be obtained by chemical synthesis methods or by recombinant methods.

Nucleic acids of the disclosure, either their entire sequence, or any portion thereof, may be non-naturally occurring. Nucleic acids of the disclosure may contain one or more mutations, substitutions, deletions, or insertions that do not naturally-occur, rendering the entire nucleic acid sequence non-naturally occurring. Nucleic acids of the disclosure may contain one or more duplicated, inverted or repeated sequences, the resultant sequence of which does not naturally-occur, rendering the entire nucleic acid sequence non-naturally occurring. Nucleic acids of the disclosure may contain modified, artificial, or synthetic nucleotides that do not naturally-occur, rendering the entire nucleic acid sequence non-naturally occurring.

Given the redundancy in the genetic code, a plurality of nucleotide sequences may encode any particular protein. All such nucleotides sequences are contemplated herein.

As used throughout the disclosure, the term “operably linked” refers to the expression of a gene that is under the control of a promoter with which it is spatially connected. A promoter can be positioned 5′ (upstream) or 3′ (downstream) of a gene under its control. The distance between a promoter and a gene can be approximately the same as the distance between that promoter and the gene it controls in the gene from which the promoter is derived. Variation in the distance between a promoter and a gene can be accommodated without loss of promoter function.

As used throughout the disclosure, the term “promoter” refers to a synthetic or naturally-derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, EF-1 Alpha promoter, CAG promoter, SV40 early promoter or SV40 late promoter and the CMV IE promoter.

As used throughout the disclosure, the term “substantially complementary” refers to a first sequence that is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 180, 270, 360, 450, 540, or more nucleotides or amino acids, or that the two sequences hybridize under stringent hybridization conditions.

As used throughout the disclosure, the term “substantially identical” refers to a first and second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 180, 270, 360, 450, 540 or more nucleotides or amino acids, or with respect to nucleic acids, if the first sequence is substantially complementary to the complement of the second sequence.

As used throughout the disclosure, the term “variant” when used to describe a nucleic acid, refers to (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

As used throughout the disclosure, the term “vector” refers to a nucleic acid sequence containing an origin of replication. A vector can be a viral vector, bacteriophage, bacterial artificial chromosome or yeast artificial chromosome. A vector can be a DNA or RNA vector. A vector can be a self-replicating extrachromosomal vector, and preferably, is a DNA plasmid. A vector may comprise a combination of an amino acid with a DNA sequence, an RNA sequence, or both a DNA and an RNA sequence.

As used throughout the disclosure, the term “variant” when used to describe a peptide or polypeptide, refers to a peptide or polypeptide that differs in amino acid sequence by the insertion, deletion, or conservative substitution of amino acids, but retain at least one biological activity. Variant can also mean a protein with an amino acid sequence that is substantially identical to a referenced protein with an amino acid sequence that retains at least one biological activity.

A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157: 105-132 (1982). The hydropathic index of an amino acid is based on a consideration of its hydrophobicity and charge. Amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference.

Substitution of amino acids having similar hydrophilicity values can result in peptides retaining biological activity, for example immunogenicity. Substitutions can be performed with amino acids having hydrophilicity values within ±2 of each other. Both the hyrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

As used herein, “conservative” amino acid substitutions may be defined as set out in Tables A, B, or C below. In some embodiments, fusion polypeptides and/or nucleic acids encoding such fusion polypeptides include conservative substitutions have been introduced by modification of polynucleotides encoding polypeptides of the invention. Amino acids can be classified according to physical properties and contribution to secondary and tertiary protein structure. A conservative substitution is a substitution of one amino acid for another amino acid that has similar properties. Exemplary conservative substitutions are set out in Table A.

TABLE A
Conservative Substitutions I

| Side chain characteristics | | Amino Acid |
|---|---|---|
| Aliphatic | Non-polar | G A P I L V F |
| | Polar-uncharged | C S T M N Q |
| | Polar-charged | D E K R |
| Aromatic | | H F W Y |
| Other | | N Q D E |

Alternately, conservative amino acids can be grouped as described in Lehninger, (Biochemistry, Second Edition; Worth Publishers, Inc. NY, N.Y. (1975), pp. 71-77) as set forth in Table B.

TABLE B
Conservative Substitutions II

| Side Chain Characteristic | | Amino Acid |
|---|---|---|
| Non-polar | Aliphatic: | A L I V P |
| (hydrophobic) | Aromatic: | F W Y |
| | Sulfur-containing: | M |
| | Borderline: | G Y |
| Uncharged-polar | Hydroxyl: | S T Y |
| | Amides: | N Q |
| | Sulfhydryl: | C |
| | Borderline: | G Y |

| | |
|---|---|
| Positively Charged (Basic): | K R H |
| Negatively Charged (Acidic): | D E |

Alternately, exemplary conservative substitutions are set out in Table C.

TABLE C
Conservative Substitutions III

| Original Residue | Exemplary Substitution |
|---|---|
| Ala (A) | Val Leu Ile Met |
| Arg (R) | Lys His |
| Asn (N) | Gln |
| Asp (D) | Glu |
| Cys (C) | Ser Thr |
| Gln (Q) | Asn |
| Glu (E) | Asp |
| Gly (G) | Ala Val Leu Pro |
| His (H) | Lys Arg |
| Ile (I) | Leu Val Met Ala Phe |
| Leu (L) | Ile Val Met Ala Phe |
| Lys (K) | Arg His |
| Met (M) | Leu Ile Val Ala |
| Phe (F) | Trp Tyr Ile |
| Pro (P) | Gly Ala Val Leu Ile |
| Ser (S) | Thr |
| Thr (T) | Ser |
| Trp (W) | Tyr Phe Ile |
| Tyr (Y) | Trp Phe Thr Ser |
| Val (V) | Ile Leu Met Ala |

It should be understood that the polypeptides of the disclosure are intended to include polypeptides bearing one or more insertions, deletions, or substitutions, or any combination thereof, of amino acid residues as well as modifications other than insertions, deletions, or substitutions of amino acid residues. Polypeptides or nucleic acids of the disclosure may contain one or more conservative substitution.

As used throughout the disclosure, the term “more than one” of the aforementioned amino acid substitutions refers to 2, 3, 4, 5, 6, 7, 8, 9, 10, 11, 12, 13, 14, 15, 16, 17, 18, 19, or 20 or more of the recited amino acid substitutions. The term “more than one” may refer to 2, 3, 4, or 5 of the recited amino acid substitutions.

Polypeptides and proteins of the disclosure, either their entire sequence, or any portion thereof, may be non-naturally occurring. Polypeptides and proteins of the disclosure may contain one or more mutations, substitutions, deletions, or insertions that do not naturally-occur, rendering the entire amino acid sequence non-naturally occurring. Polypeptides and proteins of the disclosure may contain one or more duplicated, inverted or repeated sequences, the resultant sequence of which does not naturally-occur, rendering the entire amino acid sequence non-naturally occurring. Polypeptides and proteins of the disclosure may contain modified, artificial, or synthetic amino acids that do not naturally-occur, rendering the entire amino acid sequence non-naturally occurring.

As used throughout the disclosure, “sequence identity” may be determined by using the stand-alone executable BLAST engine program for blasting two sequences (bl2seq), which can be retrieved from the National Center for Biotechnology Information (NCBI) ftp site, using the default parameters (Tatusova and Madden, FEMS Microbiol Lett., 1999, 174, 247-250; which is incorporated herein by reference in its entirety). The terms “identical” or “identity” when used in the context of two or more nucleic acids or polypeptide sequences, refer to a specified percentage of residues that are the same over a specified region of each of the sequences. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

As used throughout the disclosure, the term “endogenous” refers to nucleic acid or protein sequence naturally associated with a target gene or a host cell into which it is introduced.

As used throughout the disclosure, the term “exogenous” refers to nucleic acid or protein sequence not naturally associated with a target gene or a host cell into which it is introduced, including non-naturally occurring multiple copies of a naturally occurring nucleic acid, e.g., DNA sequence, or naturally occurring nucleic acid sequence located in a non-naturally occurring genome location.

The disclosure provides methods of introducing a polynucleotide construct comprising a DNA sequence into a host cell. By “introducing” is intended presenting to the plant the polynucleotide construct in such a manner that the construct gains access to the interior of the host cell. The methods of the invention do not depend on a particular method for introducing a polynucleotide construct into a host cell, only that the polynucleotide construct gains access to the interior of one cell of the host. Methods for introducing polynucleotide constructs into bacteria, plants, fungi and animals are known in the art including, but not limited to, stable transformation methods, transient transformation methods, and virus-mediated methods.

### Transposons/Transposases

Exemplary transposon/transposase systems of the disclosure include, but are not limited to, piggyBac transposons and transposases, Sleeping Beauty transposons and transposases, Helraiser transposons and transposases and Tol2 transposons and transposases.

The piggyBac transposase recognizes transposon-specific inverted terminal repeat sequences (ITRs) on the ends of the transposon, and moves the contents between the ITRs into TTAA chromosomal sites. The piggyBac transposon system has no payload limit for the genes of interest that can be included between the ITRs. In certain embodiments, and, in particular, those embodiments wherein the transposon is a piggyBac transposon, the transposase is a piggyBac™ or a Super piggyBac™ (SPB) transposase. In certain embodiments, and, in particular, those embodiments wherein the transposase is a Super piggyBac™ (SPB) transposase, the sequence encoding the transposase is an mRNA sequence.

In certain embodiments of the methods of the disclosure, the transposase enzyme is a piggyBac™ (PB) transposase enzyme. The piggyBac (PB) transposase enzyme may comprise or consist of an amino acid sequence at least 75%, 80%, 85%, 90%, 95%, 99% or any percentage in between identical to:

| (SEQ ID NO: 14487) | |
|---|---|
| 1 | MGSSLDDEHI LSALLQSDDE LVGEDSDSEI SDHVSEDDVQ SDTEEAFIDE VHEVQPTSSG |
| 61 | SEILDEQNVI EQPGSSLASN RILTLPQRTI RGKNKHCWST SKSTRRSRVS ALNIVRSQRG |
| 121 | PTRMCRNIYD PLLCFKLFFT DEIISEIVKW TNAEISLKRR ESMTGATFRD TNEDEIYAFF |
| 181 | GILVMTAVRK DNHMSTDDLF DRSLSMVYVS VMSRDRFDFL IRCLRMDDKS IRPTLRENDV |
| 241 | FTPVRKIWDL FIHQCIQNYT PGAHLTIDEQ LLGFRGRCPF RMYIPNKPSK YGIKILMMCD |
| 301 | SGTKYMINGM PYLGRGTQTN GVPLGEYYVK ELSKPVHGSC RNITCDNWFT SIPLAKNLLQ |
| 361 | EPYKLTIVGT VRSNKREIPE VLKNSRSRPV GTSMFCFDGP LTLVSYKPKP AKMVYLLSSC |
| 421 | DEDASINEST GKPQMVMYYN QTKGGVDTLD QMCSVMTCSR KTNRWPMALL YGMINIACIN |
| 481 | SFIIYSHNVS SKGEKVQSRK KFMRNLYMSL TSSFMRKRLE APTLKRYLRD NISNILPNEV |
| 541 | PGTSDDSTEE PVMKKRTYCT YCPSKIRRKA NASCKKCKKV ICREHNIDMC QSCF. |

In certain embodiments of the methods of the disclosure, the transposase enzyme is a piggyBac™ (PB) transposase enzyme that comprises or consists of an amino acid sequence having an amino acid substitution at one or more of positions 30, 165, 282, or 538 of the sequence:

| (SEQ ID NO: 14487) | |
|---|---|
| 1 | MGSSLDDEHI LSALLQSDDE LVGEDSDSEI SDHVSEDDVQ SDTEEAFIDE VHEVQPTSSG |
| 61 | SEILDEQNVI EQPGSSLASN RILTLPQRTI RGKNKHCWST SKSTRRSRVS ALNIVRSQRG |
| 121 | PTRMCRNIYD PLLCFKLFFT DEIISEIVKW TNAEISLKRR ESMTGATFRD TNEDEIYAFF |
| 181 | GILVMTAVRK DNHMSTDDLF DRSLSMVYVS VMSRDRFDFL IRCLRMDDKS IRPTLRENDV |
| 241 | FTPVRKIWDL FIHQCIQNYT PGAHLTIDEQ LLGFRGRCPF RMYIPNKPSK YGIKILMMCD |
| 301 | SGTKYMINGM PYLGRGTQTN GVPLGEYYVK ELSKPVHGSC RNITCDNWFT SIPLAKNLLQ |
| 361 | EPYKLTIVGT VRSNKREIPE VLKNSRSRPV GTSMFCFDGP LTLVSYKPKP AKMVYLLSSC |
| 421 | DEDASINEST GKPQMVMYYN QTKGGVDTLD QMCSVMTCSR KTNRWPMALL YGMINIACIN |
| 481 | SFIIYSHNVS SKGEKVQSRK KFMRNLYMSL TSSFMRKRLE APTLKRYLRD NISNILPNEV |
| 541 | PGTSDDSTEE PVMKKRTYCT YCPSKIRRKA NASCKKCKKV ICREHNIDMC QSCF. |

In certain embodiments, the transposase enzyme is a piggyBac™ (PB) transposase enzyme that comprises or consists of an amino acid sequence having an amino acid substitution at two or more of positions 30, 165, 282, or 538 of the sequence of SEQ ID NO: 14487. In certain embodiments, the transposase enzyme is a piggyBac™ (PB) transposase enzyme that comprises or consists of an amino acid sequence having an amino acid substitution at three or more of positions 30, 165, 282, or 538 of the sequence of SEQ ID NO: 14487. In certain embodiments, the transposase enzyme is a piggyBac™ (PB) transposase enzyme that comprises or consists of an amino acid sequence having an amino acid substitution at each of the following positions 30, 165, 282, and 538 of the sequence of SEQ ID NO: 14487. In certain embodiments, the amino acid substitution at position 30 of the sequence of SEQ ID NO: 14487 is a substitution of a valine (V) for an isoleucine (I). In certain embodiments, the amino acid substitution at position 165 of the sequence of SEQ ID NO: 14487 is a substitution of a serine (S) for a glycine (G). In certain embodiments, the amino acid substitution at position 282 of the sequence of SEQ ID NO: 14487 is a substitution of a valine (V) for a methionine (M). In certain embodiments, the amino acid substitution at position 538 of the sequence of SEQ ID NO: 14487 is a substitution of a lysine (K) for an asparagine (N).

In certain embodiments of the methods of the disclosure, the transposase enzyme is a Super piggyBac™ (SPB) transposase enzyme. In certain embodiments, the Super piggyBac™ (SPB) transposase enzymes of the disclosure may comprise or consist of the amino acid sequence of the sequence of SEQ ID NO: 14487 wherein the amino acid substitution at position 30 is a substitution of a valine (V) for an isoleucine (I), the amino acid substitution at position 165 is a substitution of a serine (S) for a glycine (G), the amino acid substitution at position 282 is a substitution of a valine (V) for a methionine (M), and the amino acid substitution at position 538 is a substitution of a lysine (K) for an asparagine (N). In certain embodiments, the Super piggyBac™ (SPB) transposase enzyme may comprise or consist of an amino acid sequence at least 75%, 80%, 85%, 90%, 95%, 99% or any percentage in between identical to:

| (SEQ ID NO: 14484) | |
|---|---|
| 1 | MGSSLDDEHI LSALLQSDDE LVGEDSDSEV SDHVSEDDVQ SDTEEAFIDE VHEVQPTSSG |
| 61 | SEILDEQNVI EQPGSSLASN RILTLPQRTI RGKNKHCWST SKSTRRSRVS ALNIVRSQRG |
| 121 | PTRMCRNIYD PLLCFKLFFT DEIISEIVKW TNAEISLKRR ESMTSATFRD TNEDEIYAFF |
| 181 | GILVMTAVRK DNHMSTDDLF DRSLSMVYVS VMSRDREDFL IRCLRMDDKS IRPTLRENDV |
| 241 | FTPVRKIWDL FIHQCIQNYT PGAHLTIDEQ LLGFRGRCPF RVYIPNKPSK YGIKILMMCD |
| 301 | SGTKYMINGM PYLGRGTQTN GVPLGEYYVK ELSKPVHGSC RNITCDNWFT SIPLAKNLLQ |
| 361 | EPYKLTIVGT VRSNKREIPE VLKNSRSRPV GTSMFCFDGP LTLVSYKPKP AKMVYLLSSC |
| 421 | DEDASINEST GKPQMVMYYN QTKGGVDTLD QMCSVMTCSR KTNRWPMALL YGMINIACIN |
| 481 | SFIIYSHNVS SKGEKVQSRK KFMRNLYMSL TSSFMRKRLE APTLKRYLRD NISNILPKEV |
| 541 | PGTSDDSTEE PVMKKRTYCT YCPSKIRRKA NASCKKCKKV ICREHNIDMC QSCF. |

In certain embodiments of the methods of the disclosure, including those embodiments wherein the transposase comprises the above-described mutations at positions 30, 165, 282 and/or 538, the piggyBac™ or Super piggyBac™ transposase enzyme may further comprise an amino acid substitution at one or more of positions 3, 46, 82, 103, 119, 125, 177, 180, 185, 187, 200, 207, 209, 226, 235, 240, 241, 243, 258, 296, 298, 311, 315, 319, 327, 328, 340, 421, 436, 456, 470, 486, 503, 552, 570 and 591 of the sequence of SEQ ID NO: 14487 or SEQ ID NO: 14484. In certain embodiments, including those embodiments wherein the transposase comprises the above-described mutations at positions 30, 165, 282 and/or 538, the piggyBac™ or Super piggyBac™ transposase enzyme may further comprise an amino acid substitution at one or more of positions 46, 119, 125, 177, 180, 185, 187, 200, 207, 209, 226, 235, 240, 241, 243, 296, 298, 311, 315, 319, 327, 328, 340, 421, 436, 456, 470, 485, 503, 552 and 570. In certain embodiments, the amino acid substitution at position 3 of SEQ ID NO: 14487 or SEQ ID NO: 14484 is a substitution of an asparagine (N) for a serine (S). In certain embodiments, the amino acid substitution at position 46 of SEQ ID NO: 14487 or SEQ ID NO: 14484 is a substitution of a serine (S) for an alanine (A). In certain embodiments, the amino acid substitution at position 46 of SEQ ID NO: 14487 or SEQ ID NO: 14484 is a substitution of a threonine (T) for an alanine (A). In certain embodiments, the amino acid substitution at position 82 of SEQ ID NO: 14487 or SEQ ID NO: 14484 is a substitution of a tryptophan (W) for an isoleucine (I). In certain embodiments, the amino acid substitution at position 103 of SEQ ID NO: 14487 or SEQ ID NO: 14484 is a substitution of a proline (P) for a serine (S). In certain embodiments, the amino acid substitution at position 119 of SEQ ID NO: 14487 or SEQ ID NO: 14484 is a substitution of a proline (P) for an arginine (R). In certain embodiments, the amino acid substitution at position 125 of SEQ ID NO: 14487 or SEQ ID NO: 14484 is a substitution of an alanine (A) a cysteine (C). In certain embodiments, the amino acid substitution at position 125 of SEQ ID NO: 14487 or SEQ ID NO: 14484 is a substitution of a leucine (L) for a cysteine (C). In certain embodiments, the amino acid substitution at position 177 of SEQ ID NO: 14487 or SEQ ID NO: 14484 is a substitution of a lysine (K) for a tyrosine (Y). In certain embodiments, the amino acid substitution at position 177 of SEQ ID NO: 14487 or SEQ ID NO: 14484 is a substitution of a histidine (H) for a tyrosine (Y). In certain embodiments, the amino acid substitution at position 180 of SEQ ID NO: 14487 or SEQ ID NO: 14484 is a substitution of a leucine (L) for a phenylalanine (F). In certain embodiments, the amino acid substitution at position 180 of SEQ ID NO: 14487 or SEQ ID NO: 14484 is a substitution of an isoleucine (I) for a phenylalanine (F). In certain embodiments, the amino acid substitution at position 180 of SEQ ID NO: 14487 or SEQ ID NO: 14484 is a substitution of a valine (V) for a phenylalanine (F). In certain embodiments, the amino acid substitution at position 185 of SEQ ID NO: 14487 or SEQ ID NO: 14484 is a substitution of a leucine (L) for a methionine (M). In certain embodiments, the amino acid substitution at position 187 of SEQ ID NO: 14487 or SEQ ID NO: 14484 is a substitution of a glycine (G) for an alanine (A). In certain embodiments, the amino acid substitution at position 200 of SEQ ID NO: 14487 or SEQ ID NO: 14484 is a substitution of a tryptophan (W) for a phenylalanine (F). In certain embodiments, the amino acid substitution at position 207 of SEQ ID NO: 14487 or SEQ ID NO: 14484 is a substitution of a proline (P) for a valine (V). In certain embodiments, the amino acid substitution at position 209 of SEQ ID NO: 14487 or SEQ ID NO: 14484 is a substitution of a phenylalanine (F) for a valine (V). In certain embodiments, the amino acid substitution at position 226 of SEQ ID NO: 14487 or SEQ ID NO: 14484 is a substitution of a phenylalanine (F) for a methionine (M). In certain embodiments, the amino acid substitution at position 235 of SEQ ID NO: 14487 or SEQ ID NO: 14484 is a substitution of an arginine (R) for a leucine (L). In certain embodiments, the amino acid substitution at position 240 of SEQ ID NO: 14487 or SEQ ID NO: 14484 is a substitution of a lysine (K) for a valine (V). In certain embodiments, the amino acid substitution at position 241 of SEQ ID NO: 14487 or SEQ ID NO: 14484 is a substitution of a leucine (L) for a phenylalanine (F). In certain embodiments, the amino acid substitution at position 243 of SEQ ID NO: 14487 or SEQ ID NO: 14484 is a substitution of a lysine (K) for a proline (P). In certain embodiments, the amino acid substitution at position 258 of SEQ ID NO: 14487 or SEQ ID NO: 14484 is a substitution of a serine (S) for an asparagine (N). In certain embodiments, the amino acid substitution at position 296 of SEQ ID NO: 14487 or SEQ ID NO: 14484 is a substitution of a tryptophan (W) for a leucine (L). In certain embodiments, the amino acid substitution at position 296 of SEQ ID NO: 14487 or SEQ ID NO: 14484 is a substitution of a tyrosine (Y) for a leucine (L). In certain embodiments, the amino acid substitution at position 296 of SEQ ID NO: 14487 or SEQ ID NO: 14484 is a substitution of a phenylalanine (F) for a leucine (L). In certain embodiments, the amino acid substitution at position 298 of SEQ ID NO: 14487 or SEQ ID NO: 14484 is a substitution of a leucine (L) for a methionine (M). In certain embodiments, the amino acid substitution at position 298 of SEQ ID NO: 14487 or SEQ ID NO: 14484 is a substitution of an alanine (A) for a methionine (M). In certain embodiments, the amino acid substitution at position 298 of SEQ ID NO: 14487 or SEQ ID NO: 14484 is a substitution of a valine (V) for a methionine (M). In certain embodiments, the amino acid substitution at position 311 of SEQ ID NO: 14487 or SEQ ID NO: 14484 is a substitution of an isoleucine (I) for a proline (P). In certain embodiments, the amino acid substitution at position 311 of SEQ ID NO: 14487 or SEQ ID NO: 14484 is a substitution of a valine for a proline (P). In certain embodiments, the amino acid substitution at position 315 of SEQ ID NO: 14487 or SEQ ID NO: 14484 is a substitution of a lysine (K) for an arginine (R). In certain embodiments, the amino acid substitution at position 319 of SEQ ID NO: 14487 or SEQ ID NO: 14484 is a substitution of a glycine (G) for a threonine (T). In certain embodiments, the amino acid substitution at position 327 of SEQ ID NO: 14487 or SEQ ID NO: 14484 is a substitution of an arginine (R) for a tyrosine (Y). In certain embodiments, the amino acid substitution at position 328 of SEQ ID NO: 14487 or SEQ ID NO: 14484 is a substitution of a valine (V) for a tyrosine (Y). In certain embodiments, the amino acid substitution at position 340 of SEQ ID NO: 14487 or SEQ ID NO: 14484 is a substitution of a glycine (G) for a cysteine (C). In certain embodiments, the amino acid substitution at position 340 of SEQ ID NO: 14487 or SEQ ID NO: 14484 is a substitution of a leucine (L) for a cysteine (C). In certain embodiments, the amino acid substitution at position 421 of SEQ ID NO: 14487 or SEQ ID NO: 14484 is a substitution of a histidine (H) for the aspartic acid (D). In certain embodiments, the amino acid substitution at position 436 of SEQ ID NO: 14487 or SEQ ID NO: 14484 is a substitution of an isoleucine (I) for a valine (V). In certain embodiments, the amino acid substitution at position 456 of SEQ ID NO: 14487 or SEQ ID NO: 14484 is a substitution of a tyrosine (Y) for a methionine (M). In certain embodiments, the amino acid substitution at position 470 of SEQ ID NO: 14487 or SEQ ID NO: 14484 is a substitution of a phenylalanine (F) for a leucine (L). In certain embodiments, the amino acid substitution at position 485 of SEQ ID NO: 14487 or SEQ ID NO: 14484 is a substitution of a lysine (K) for a serine (S). In certain embodiments, the amino acid substitution at position 503 of SEQ ID NO: 14487 or SEQ ID NO: 14484 is a substitution of a leucine (L) for a methionine (M). In certain embodiments, the amino acid substitution at position 503 of SEQ ID NO: 14487 or SEQ ID NO: 14484 is a substitution of an isoleucine (I) for a methionine (M). In certain embodiments, the amino acid substitution at position 552 of SEQ ID NO: 14487 or SEQ ID NO: 14484 is a substitution of a lysine (K) for a valine (V). In certain embodiments, the amino acid substitution at position 570 of SEQ ID NO: 14487 or SEQ ID NO: 14484 is a substitution of a threonine (T) for an alanine (A). In certain embodiments, the amino acid substitution at position 591 of SEQ ID NO: 14487 or SEQ ID NO: 14484 is a substitution of a proline (P) for a glutamine (Q). In certain embodiments, the amino acid substitution at position 591 of SEQ ID NO: 14487 or SEQ ID NO: 14484 is a substitution of an arginine (R) for a glutamine (Q).

In certain embodiments of the methods of the disclosure, including those embodiments wherein the transposase comprises the above-described mutations at positions 30, 165, 282 and/or 538, the piggyBac™ transposase enzyme may comprise or the Super piggyBac™ transposase enzyme may further comprise an amino acid substitution at one or more of positions 103, 194, 372, 375, 450, 509 and 570 of the sequence of SEQ ID NO: 14487 or SEQ ID NO: 14484. In certain embodiments of the methods of the disclosure, including those embodiments wherein the transposase comprises the above-described mutations at positions 30, 165, 282 and/or 538, the piggyBac™ transposase enzyme may comprise or the Super piggyBac™ transposase enzyme may further comprise an amino acid substitution at two, three, four, five, six or more of positions 103, 194, 372, 375, 450, 509 and 570 of the sequence of SEQ ID NO: 14487 or SEQ ID NO: 14484. In certain embodiments, including those embodiments wherein the transposase comprises the above-described mutations at positions 30, 165, 282 and/or 538, the piggyBac™ transposase enzyme may comprise or the Super piggyBac™ transposase enzyme may further comprise an amino acid substitution at positions 103, 194, 372, 375, 450, 509 and 570 of the sequence of SEQ ID NO: 14487 or SEQ ID NO: 14484. In certain embodiments, the amino acid substitution at position 103 of SEQ ID NO: 14487 or SEQ ID NO: 14484 is a substitution of a proline (P) for a serine (S). In certain embodiments, the amino acid substitution at position 194 of SEQ ID NO: 14487 or SEQ ID NO: 14484 is a substitution of a valine (V) for a methionine (M). In certain embodiments, the amino acid substitution at position 372 of SEQ ID NO: 14487 or SEQ ID NO: 14484 is a substitution of an alanine (A) for an arginine (R). In certain embodiments, the amino acid substitution at position 375 of SEQ ID NO: 14487 or SEQ ID NO: 14484 is a substitution of an alanine (A) for a lysine (K). In certain embodiments, the amino acid substitution at position 450 of SEQ ID NO: 14487 or SEQ ID NO: 14484 is a substitution of an asparagine (N) for an aspartic acid (D). In certain embodiments, the amino acid substitution at position 509 of SEQ ID NO: 14487 or SEQ ID NO: 14484 is a substitution of a glycine (G) for a serine (S). In certain embodiments, the amino acid substitution at position 570 of SEQ ID NO: 14487 or SEQ ID NO: 14484 is a substitution of a serine (S) for an asparagine (N). In certain embodiments, the piggyBac™ transposase enzyme may comprise a substitution of a valine (V) for a methionine (M) at position 194 of SEQ ID NO: 14487. In certain embodiments, including those embodiments wherein the piggyBac™ transposase enzyme may comprise a substitution of a valine (V) for a methionine (M) at position 194 of SEQ ID NO: 14487, the piggyBac™ transposase enzyme may further comprise an amino acid substitution at positions 372, 375 and 450 of the sequence of SEQ ID NO: 14487 or SEQ ID NO: 14484. In certain embodiments, the piggyBac™ transposase enzyme may comprise a substitution of a valine (V) for a methionine (M) at position 194 of SEQ ID NO: 14487, a substitution of an alanine (A) for an arginine (R) at position 372 of SEQ ID NO: 14487, and a substitution of an alanine (A) for a lysine (K) at position 375 of SEQ ID NO: 14487. In certain embodiments, the piggyBac™ transposase enzyme may comprise a substitution of a valine (V) for a methionine (M) at position 194 of SEQ ID NO: 14487, a substitution of an alanine (A) for an arginine (R) at position 372 of SEQ ID NO: 14487, a substitution of an alanine (A) for a lysine (K) at position 375 of SEQ ID NO: 14487 and a substitution of an asparagine (N) for an aspartic acid (D) at position 450 of SEQ ID NO: 14487.

The sleeping beauty transposon is transposed into the target genome by the Sleeping Beauty transposase that recognizes ITRs, and moves the contents between the ITRs into TA chromosomal sites. In various embodiments, SB transposon-mediated gene transfer, or gene transfer using any of a number of similar transposons, may be used in the compositions and methods of the disclosure.

In certain embodiments, and, in particular, those embodiments wherein the transposon is a Sleeping Beauty transposon, the transposase is a Sleeping Beauty transposase or a hyperactive Sleeping Beauty transposase (SB100X).

In certain embodiments of the methods of the disclosure, the Sleeping Beauty transposase enzyme comprises an amino acid sequence at least 75%, 80%, 85%, 90%, 95%, 99% or any percentage in between identical to:

| (SEQ ID NO: 14485) | | |
|---|---|---|
| 1 | MGKSKEISQD LRKKIVDLHK | SGSSLGAISK RLKVPRSSVQ TIVRKYKHHG TTQPSYRSGR |
| 61 | RRVLSPRDER TLVRKVQINP | RTTAKDLVKM LEETGTKVSI STVKRVLYRH NLKGRSARKK |
| 121 | PLLQNRHKKA RLRFATAHGD | KDRTFWRNVL WSDETKIELF GHNDHRYVWR KKGEACKPKN |
| 181 | TIPTVKHGGG SIMLWGCFAA | GGTGALHKID GIMRKENYVD ILKQHLKTSV RKLKLGRKWV |
| 241 | FQMDNDPKHT SKVVAKWLKD | NKVKVLEWPS QSPDLNPIEN LWAELKKRVR ARRPTNLTQL |
| 301 | HQLCQEEWAK IHPTYCGKLV | EGYPKRLTQV KQFKGNATKY. |

In certain embodiments of the methods of the disclosure, the hyperactive Sleeping Beauty (SB100X) transposase enzyme comprises an amino acid sequence at least 75%, 80%, 85%, 90%, 95%, 99% or any percentage in between identical to:

| (SEQ ID NO: 14486) | |
|---|---|
| 1 | MGKSKEISQD LRKRIVDLHK SGSSLGAISK RLAVPRSSVQ TIVRKYKHHG TTQPSYRSGR |
| 61 | RRVLSPRDER TLVRKVQINP RTTAKDLVKM LEETGTKVSI STVKRVLYRH NLKGHSARKK |
| 121 | PLLQNRHKKA RLRFATAHGD KDRTFWRNVL WSDETKIELF GHNDHRYVWR KKGEACKPKN |
| 181 | TIPTVKHGGG SIMLWGCFAA GGTGALHKID GIMDAVQYVD ILKQHLKTSV RKLKLGRKWV |
| 241 | FQHDNDPKHT SKVVAKWLKD NKVKVLEWPS QSPDLNPIEN LWAELKKRVR ARRPTNLTQL |
| 301 | HQLCQEEWAK IHPNYCGKLV EGYPKRLTQV KQFKGNATKY. |

The Helraiser transposon is transposed by the Helitron transposase. Helitron transposases mobilize the Helraiser transposon, an ancient element from the bat genome that was active about 30 to 36 million years ago. An exemplary Helraiser transposon of the disclosure includes Helibat1, which comprises a nucleic acid sequence comprising:

| (SEQ ID NO: 14652) | |
|---|---|
| 1 | TCCTATATAA TAAAAGAGAA ACATGCAAAT TGACCATCCC TCCGCTACGC TCAAGCCACG |
| 61 | CCCACCAGCC AATCAGAAGT GACTATGCAA ATTAACCCAA CAAAGATGGC AGTTAAATTT |
| 121 | GCATACGCAG GTGTCAAGCG CCCCAGGAGG CAACGGCGGC CGCGGGCTCC CAGGACCTTC |
| 181 | GCTGGCCCCG GGAGGCGAGG CCGGCCGCGC CTAGCCACAC CCGCGGGCTC CCGGGACCTT |
| 241 | CGCCAGCAGA GAGCAGAGCG GGAGAGCGGG CGGAGAGCGG GAGGTTTGGA GGACTTGGCA |
| 301 | GAGCAGGAGG CCGCTGGACA TAGAGCAGAG CGAGAGAGAG GGTGGCTTGG AGGGCGTGGC |
| 361 | TCCCTCTGTC ACCCCAGCTT CCTCATCACA GCTGTGGAAA CTGACAGCAG GGAGGAGGAA |
| 421 | GTCCCACCCC CACAGAATCA GCCAGAATCA GCCGTTGGTC AGACAGCTCT CAGCGGCCTG |
| 481 | ACAGCCAGGA CTCTCATTCA CCTGCATCTC AGACCGTGAC AGTAGAGAGG TGGGACTATG |
| 541 | TCTAAAGAAC AACTGTTGAT ACAACGTAGC TCTGCAGCCG AAAGATGCCG GCGTTATCGA |
| 601 | CAGAAAATGT CTGCAGAGCA ACGTGCGTCT GATCTTGAAA GAAGGCGGCG CCTGCAACAG |
| 661 | AATGTATCTG AAGAGCAGCT ACTGGAAAAA CGTCGCTCTG AAGCCGAAAA ACAGCGGCGT |
| 721 | CATCGACAGA AAATGTCTAA AGACCAACGT GCCTTTGAAG TTGAAAGAAG GCGGTGGCGA |
| 781 | CGACAGAATA TGTCTAGAGA ACAGTCATCA ACAAGTACTA CCAATACCGG TAGGAACTGC |
| 841 | CTTCTCAGCA AAAATGGAGT ACATGAGGAT GCAATTCTCG AACATAGTTG TGGTGGAATG |
| 901 | ACTGTTCGAT GTGAATTTTG CCTATCACTA AATTTCTCTG ATGAAAAACC ATCCGATGGG |
| 961 | AAATTTACTC GATGTTGTAG CAAAGGGAAA GTCTGTCCAA ATGATATACA TTTTCCAGAT |
| 1021 | TACCCGGCAT ATTTAAAAAG ATTAATGACA AACGAAGATT CTGACAGTAA AAATTTCATG |
| 1081 | GAAAATATTC GTTCCATAAA TAGTTCTTTT GCTTTTGCTT CCATGGGTGC AAATATTGCA |
| 1141 | TCGCCATCAG GATATGGGCC ATACTGTTTT AGAATACACG GACAAGTTTA TCACCGTACT |
| 1201 | GGAACTTTAC ATCCTTCGGA TGGTGTTTCT CGGAAGTTTG CTCAACTCTA TATTTTGGAT |
| 1261 | ACAGCCGAAG CTACAAGTAA AAGATTAGCA ATGCCAGAAA ACCAGGGCTG CTCAGAAAGA |
| 1321 | CTCATGATCA ACATCAACAA CCTCATGCAT GAAATAAATG AATTAACAAA ATCGTACAAG |
| 1381 | ATGCTACATG AGGTAGAAAA GGAAGCCCAA TCTGAAGCAG CAGCAAAAGG TATTGCTCCC |
| 1441 | ACAGAAGTAA CAATGGCGAT TAAATACGAT CGTAACAGTG ACCCAGGTAG ATATAATTCT |
| 1501 | CCCCGTGTAA CCGAGGTTGC TGTCATATTC AGAAACGAAG ATGGAGAACC TCCTTTTGAA |
| 1561 | AGGGACTTGC TCATTCATTG TAAACCAGAT CCCAATAATC CAAATGCCAC TAAAATGAAA |
| 1621 | CAAATCAGTA TCCTGTTTCC TACATTAGAT GCAATGACAT ATCCTATTCT TTTTCCACAT |
| 1681 | GGTGAAAAAG GCTGGGGAAC AGATATTGCA TTAAGACTCA GAGACAACAG TGTAATCGAC |
| 1741 | AATAATACTA GACAAAATGT AAGGACACGA GTCACACAAA TGCAGTATTA TGGATTTCAT |
| 1801 | CTCTCTGTGC GGGACACGTT CAATCCTATT TTAAATGCAG GAAAATTAAC TCAACAGTTT |
| 1861 | ATTGTGGATT CATATTCAAA AATGGAGGCC AATCGGATAA ATTTCATCAA AGCAAACCAA |
| 1921 | TCTAAGTTGA GAGTTGAAAA ATATAGTGGT TTGATGGATT ATCTCAAATC TAGATCTGAA |
| 1981 | AATGACAATG TGCCGATTGG TAAAATGATA ATACTTCCAT CATCTTTTGA GGGTAGTCCC |
| 2041 | AGAAATATGC AGCAGCGATA TCAGGATGCT ATGGCAATTG TAACGAAGTA TGGCAAGCCC |
| 2101 | GATTTATTCA TAACCATGAC ATGCAACCCC AAATGGGCAG ATATTACAAA CAATTTACAA |
| 2161 | CGCTGGCAAA AAGTTGAAAA CAGACCTGAC TTGGTAGCCA GAGTTTTTAA TATTAAGCTG |
| 2221 | AATGCTCTTT TAAATGATAT ATGTAAATTC CATTTATTTG GCAAAGTAAT AGCTAAAATT |
| 2281 | CATGTCATTG AATTTCAGAA ACGCGGACTG CCTCACGCTC ACATATTATT GATATTAGAT |
| 2341 | AGTGAGTCCA AATTACGTTC AGAAGATGAC ATTGACCGTA TAGTTAAGGC AGAAATTCCA |
| 2401 | GATGAAGACC AGTGTCCTCG ACTTTTTCAA ATTGTAAAAT CAAATATGGT ACATGGACCA |
| 2461 | TGTGGAATAC AAAATCCAAA TAGTCCATGT ATGGAAAATG GAAAATGTTC AAAGGGATAT |
| 2521 | CCAAAAGAAT TTCAAAATGC GACCATTGGA AATATTGATG GATATCCCAA ATACAAACGA |
| 2581 | AGATCTGGTA GCACCATGTC TATTGGAAAT AAAGTTGTCG ATAACACTTG GATTGTCCCT |
| 2641 | TATAACCCGT ATTTGTGCCT TAAATATAAC TGTCATATAA ATGTTGAAGT CTGTGCATCA |
| 2701 | ATTAAAAGTG TCAAATATTT ATTTAAATAC ATCTATAAAG GGCACGATTG TGCAAATATT |
| 2761 | CAAATTTCTG AAAAAAATAT TATCAATCAT GACGAAGTAC AGGACTTCAT TGACTCCAGG |
| 2821 | TATGTGAGCG CTCCTGAGGC TGTTTGGAGA CTTTTTGCAA TGCGAATGCA TGACCAATCT |
| 2881 | CATGCAATCA CAAGATTAGC TATTCATTTG CCAAATGATC AGAATTTGTA TTTTCATACC |
| 2941 | GATGATTTTG CTGAAGTTTT AGATAGGGCT AAAAGGCATA ACTCGACTTT GATGGCTTGG |
| 3001 | TTCTTATTGA ATAGAGAAGA TTCTGATGCA CGTAATTATT ATTATTGGGA GATTCCACAG |
| 3061 | CATTATGTGT TTAATAATTC TTTGTGGACA AAACGCCGAA AGGGTGGGAA TAAAGTATTA |
| 3121 | GGTAGACTGT TCACTGTGAG CTTTAGAGAA CCAGAACGAT ATTACCTTAG ACTTTTGCTT |
| 3181 | CTGCATGTAA AAGGTGCGAT AAGTTTTGAG GATCTGCGAA CTGTAGGAGG TGTAACTTAT |
| 3241 | GATACATTTC ATGAAGCTGC TAAACACCGA GGATTATTAC TTGATGACAC TATCTGGAAA |
| 3301 | GATACGATTG ACGATGCAAT CATCCTTAAT ATGCCCAAAC AACTACGGCA ACTTTTTGCA |
| 3361 | TATATATGTG TGTTTGGATG TCCTTCTGCT GCAGACAAAT TATGGGATGA GAATAAATCT |
| 3421 | CATTTTATTG AAGATTTCTG TTGGAAATTA CACCGAAGAG AAGGTGCCTG TGTGAACTGT |
| 3481 | GAAATGCATG CCCTTAACGA AATTCAGGAG GTATTCACAT TGCATGGAAT GAAATGTTCA |
| 3541 | CATTTCAAAC TTCCGGACTA TCCTTTATTA ATGAATGCAA ATACATGTGA TCAATTGTAC |
| 3601 | GAGCAACAAC AGGCAGAGGT TTTGATAAAT TCTCTGAATG ATGAACAGTT GGCAGCCTTT |
| 3661 | CAGACTATAA CTTCAGCCAT CGAAGATCAA ACTGTACACC CCAAATGCTT TTTCTTGGAT |
| 3721 | GGTCCAGGTG GTAGTGGAAA AACATATCTG TATAAAGTTT TAACACATTA TATTAGAGGT |
| 3781 | CGTGGTGGTA CTGTTTTACC CACAGCATCT ACAGGAATTG CTGCAAATTT ACTTCTTGGT |
| 3841 | GGAAGAACCT TTCATTCCCA ATATAAATTA CCAATTCCAT TAAATGAAAC TTCAATTTCT |
| 3901 | AGACTCGATA TAAAGAGTGA AGTTGCTAAA ACCATTAAAA AGGCCCAACT TCTCATTATT |
| 3961 | GATGAATGCA CCATGGCATC CAGTCATGCT ATAAACGCCA TAGATAGATT ACTAAGAGAA |
| 4021 | ATTATGAATT TGAATGTTGC ATTTGGTGGG AAAGTTCTCC TTCTCGGAGG GGATTTTCGA |
| 4081 | CAATGTCTCA GTATTGTACC ACATGCTATG CGATCGGCCA TAGTACAAAC GAGTTTAAAG |
| 4141 | TACTGTAATG TTTGGGGATG TTTCAGAAAG TTGTCTCTTA AAACAAATAT GAGATCAGAG |
| 4201 | GATTCTGCTT ATAGTGAATG GTTAGTAAAA CTTGGAGATG GCAAACTTGA TAGCAGTTTT |
| 4261 | CATTTAGGAA TGGATATTAT TGAAATCCCC CATGAAATGA TTTGTAACGG ATCTATTATT |
| 4321 | GAAGCTACCT TTGGAAATAG TATATCTATA GATAATATTA AAAATATATC TAAACGTGCA |
| 4381 | ATTCTTTGTC CAAAAAATGA GCATGTTCAA AAATTAAATG AAGAAATTTT GGATATACTT |
| 4441 | GATGGAGATT TTCACACATA TTTGAGTGAT GATTCCATTG ATTCAACAGA TGATGCTGAA |
| 4501 | AAGGAAAATT TTCCCATCGA ATTTCTTAAT AGTATTACTC CTTCGGGAAT GCCGTGTCAT |
| 4561 | AAATTAAAAT TGAAAGTGGG TGCAATCATC ATGCTATTGA GAAATCTTAA TAGTAAATGG |
| 4621 | GGTCTTTGTA ATGGTACTAG ATTTATTATC AAAAGATTAC GACCTAACAT TATCGAAGCT |
| 4681 | GAAGTATTAA CAGGATCTGC AGAGGGAGAG GTTGTTCTGA TTCCAAGAAT TGATTTGTCC |
| 4741 | CCATCTGACA CTGGCCTCCC ATTTAAATTA ATTCGAAGAC AGTTTCCCGT GATGCCAGCA |
| 4801 | TTTGCGATGA CTATTAATAA ATCACAAGGA CAAACTCTAG ACAGAGTAGG AATATTCCTA |
| 4861 | CCTGAACCCG TTTTCGCACA TGGTCAGTTA TATGTTGCTT TCTCTCGAGT TCGAAGAGCA |
| 4921 | TGTGACGTTA AAGTTAAAGT TGTAAATACT TCATCACAAG GGAAATTAGT CAAGCACTCT |
| 4981 | GAAAGTGTTT TTACTCTTAA TGTGGTATAC AGGGAGATAT TAGAATAAGT TTAATCACTT |
| 5041 | TATCAGTCAT TGTTTGCATC AATGTTGTTT TTATATCATG TTTTTGTTGT TTTTATATCA |
| 5101 | TGTCTTTGTT GTTGTTATAT CATGTTGTTA TTGTTTATTT ATTAATAAAT TTATGTATTA |
| 5161 | TTTTCATATA CATTTTACTC ATTTCCTTTC ATCTCTCACA CTTCTATTAT AGAGAAAGGG |
| 5221 | CAAATAGCAA TATTAAAATA TTTCCTCTAA TTAATTCCCT TTCAATGTGC ACGAATTTCG |
| 5281 | TGCACCGGGC CACTAG. |

Unlike other transposases, the Helitron transposase does not contain an RNase-H like catalytic domain, but instead comprises a RepHel motif made up of a replication initiator domain (Rep) and a DNA helicase domain. The Rep domain is a nuclease domain of the HUH superfamily of nucleases.

An exemplary Helitron transposase of the disclosure comprises an amino acid sequence comprising:

| (SEQ ID NO: 14501) | |
|---|---|
| 1 | MSKEQLLIQR SSAAERCRRY RQKMSAEQRA SDLERRRRLQ QNVSEEQLLE KRRSEAEKQR |
| 61 | RHRQKMSKDQ RAFEVERRRW RRQNMSREQS STSTTNTGRN CLLSKNGVHE DAILEHSCGG |
| 121 | MTVRCEFCLS LNFSDEKPSD GKFTRCCSKG KVCPNDIHFP DYPAYLKRLM TNEDSDSKNF |
| 181 | MENIRSINSS FAFASMGANI ASPSGYGPYC FRIHGQVYHR TGTLHPSDGV SRKFAQLYIL |
| 241 | DTAEATSKRL AMPENQGCSE RLMININNLM HEINELTKSY KMLHEVEKEA QSEAAAKGIA |
| 301 | PTEVIMAIKY DRNSDPGRYN SPRVTEVAVI FRNEDGEPPF ERDLLIHCKP DPNNPNATKM |
| 361 | KQISILFPTL DAMTYPILFP HGEKGWGTDI ALRLRDNSVI DNNTRQNVRT RVTQMQYYGF |
| 421 | HLSVRDTFNP ILNAGKLTQQ FIVDSYSKME ANRINFIKAN QSKLRVEKYS GLMDYLKSRS |
| 481 | ENDNVPIGKM IILPSSFEGS PRNMQQRYQD AMAIVTKYGK PDLFITMTCN PKWADITNNL |
| 541 | QRWQKVENRP DLVARVFNIK LNALLNDICK FHLFGKVIAK IHVIEFQKRG LPHAHILLIL |
| 601 | DSESKLRSED DIDRIVKAEI PDEDQCPRLF QIVKSNMVHG PCGIQNPNSP CMENGKCSKG |
| 661 | YPKEFQNATI GNIDGYPKYK RRSGSTMSIG NKVVDNTWIV PYNPYLCLKY NCHINVEVCA |
| 721 | SIKSVKYLFK YIYKGHDCAN IQISEKNIIN HDEVQDFIDS RYVSAPEAVW RLFAMRMHDQ |
| 781 | SHAITRLAIH LPNDQNLYFH TDDFAEVLDR AKRHNSTLMA WFLLNREDSD ARNYYYWEIP |
| 841 | QHYVFNNSLW TKRRKGGNKV LGRLFTVSFR EPERYYLRLL LLHVKGAISF EDLRTVGGVT |
| 901 | YDTFHEAAKH RGLLLDDTIW KDTIDDAIIL NMPKQLRQLF AYICVFGCPS AADKLWDENK |
| 961 | SHFIEDFCWK LHRREGACVN CEMHALNEIQ EVFTLHGMKC SHFKLPDYPL LMNANTCDQL |
| 1021 | YEQQQAEVLI NSLNDEQLAA FQTITSAIED QTVHPKCFFL DGPGGSGKTY LYKVLTHYIR |
| 1081 | GRGGTVLPTA STGIAANLLL GGRTFHSQYK LPIPLNETSI SRLDIKSEVA KTIKKAQLLI |
| 1141 | IDECTMASSH AINAIDRLLR EIMNLNVAFG GKVLLLGGDF RQCLSIVPHA MRSAIVQTSL |
| 1201 | KYCNVWGCFR KLSLKTNMRS EDSAYSEWLV KLGDGKLDSS FHLGMDIIEI PHEMICNGSI |
| 1261 | IEATFGNSIS IDNIKNISKR AILCPKNEHV QKLNEEILDI LDGDFHTYLS DDSIDSTDDA |
| 1321 | EKENFPIEFL NSITPSGMPC HKLKLKVGAI IMLLRNLNSK WGLCNGTRFI IKRLRPNIIE |
| 1381 | AEVLTGSAEG EVVLIPRIDL SPSDTGLPFK LIRRQFPVMP AFAMTINKSQ GQTLDRVGIF |
| 1441 | LPEPVFAHGQ LYVAFSRVRR ACDVKVKVVN TSSQGKLVKH SESVFTLNVV YREILE. |

In Helitron transpositions, a hairpin close to the 3′ end of the transposon functions as a terminator. However, this hairpin can be bypassed by the transposase, resulting in the transduction of flanking sequences. In addition, Helraiser transposition generates covalently closed circular intermediates. Furthermore, Helitron transpositions can lack target site duplications. In the Helraiser sequence, the transposase is flanked by left and right terminal sequences termed LTS and RTS. These sequences terminate with a conserved 5′-TC/CTAG-3′ motif. A 19 bp palindromic sequence with the potential to form the hairpin termination structure is located 11 nucleotides upstream of the RTS and consists of the sequence

(SEQ ID NO: 14500)
GTGCACGAATTTCGTGCACCGGGCCACTAG.

Tol2 transposons may be isolated or derived from the genome of the medaka fish, and may be similar to transposons of the hAT family. Exemplary Tol2 transposons of the disclosure are encoded by a sequence comprising about 4.7 kilobases and contain a gene encoding the Tol2 transposase, which contains four exons. An exemplary Tol2 transposase of the disclosure comprises an amino acid sequence comprising the following:

| (SEQ ID NO: 14502) | |
|---|---|
| 1 | MEEVCDSSAA ASSTVQNQPQ DQEHPWPYLR EFFSLSGVNK DSFKMKCVLC LPLNKEISAF |
| 61 | KSSPSNLRKH IERMHPNYLK NYSKLTAQKR KIGTSTHASS SKQLKVDSVF PVKHVSPVTV |
| 121 | NKAILRYIIQ GLHPFSTVDL PSFKELISTL QPGISVITRP TLRSKIAEAA LIMKQKVTAA |
| 181 | MSEVEWIATT TDCWTARRKS FIGVTAHWIN PGSLERHSAA LACKRLMGSH TFEVLASAMN |
| 241 | DIHSEYEIRD KVVCTTTDSG SNFMKAFRVF GVENNDIETE ARRCESDDTD SEGCGEGSDG |
| 301 | VEFQDASRVL DQDDGFEFQL PKHQKCACHL LNLVSSVDAQ KALSNEHYKK LYRSVFGKCQ |
| 361 | ALWNKSSRSA LAAEAVESES RLQLLRPNQT RWNSTFMAVD RILQICKEAG EGALRNICTS |
| 421 | LEVPMFNPAE MLFLTEWANT MRPVAKVLDI LQAETNTQLG WLLPSVHQLS LKLQRLHHSL |
| 481 | RYCDPLVDAL QQGIQTRFKH MFEDPEITAA AILLPKFRTS WTNDETIIKR GMDYIRVHLE |
| 541 | PLDHKKELAN SSSDDEDFFA SLKPTTHEAS KELDGYLACV SDTRESLLTF PAICSLSIKT |
| 601 | NTPLPASAAC ERLFSTAGLL FSPKRARLDT NNFENQLLLK LNLRFYNFE. |

An exemplary Tol2 transposon of the disclosure, including inverted repeats, subterminal sequences and the Tol2 transposase, is encoded by a nucleic acid sequence comprising the following:

| (SEQ ID NO: 14653) | |
|---|---|
| 1 | CAGAGGTGTA AAGTACTTGA GTAATTTTAC TTGATTACTG TACTTAAGTA TTATTTTTGG |
| 61 | GGATTTTTAC TTTACTTGAG TACAATTAAA AATCAATACT TTTACTTTTA CTTAATTACA |
| 121 | TTTTTTTAGA AAAAAAAGTA CTTTTTACTC CTTACAATTT TATTTACAGT CAAAAAGTAC |
| 181 | TTATTTTTTG GAGATCACTT CATTCTATTT TCCCTTGCTA TTACCAAACC AATTGAATTG |
| 241 | CGCTGATGCC CAGTTTAATT TAAATGTTAT TTATTCTGCC TATGAAAATC GTTTTCACAT |
| 301 | TATATGAAAT TGGTCAGACA TGTTCATTGG TCCTTTGGAA GTGACGTCAT GTCACATCTA |
| 361 | TTACCACAAT GCACAGCACC TTGACCTGGA AATTAGGGAA ATTATAACAG TCAATCAGTG |
| 421 | GAAGAAAATG GAGGAAGTAT GTGATTCATC AGCAGCTGCG AGCAGCACAG TCCAAAATCA |
| 481 | GCCACAGGAT CAAGAGCACC CGTGGCCGTA TCTTCGCGAA TTCTTTTCTT TAAGTGGTGT |
| 541 | AAATAAAGAT TCATTCAAGA TGAAATGTGT CCTCTGTCTC CCGCTTAATA AAGAAATATC |
| 601 | GGCCTTCAAA AGTTCGCCAT CAAACCTAAG GAAGCATATT GAGGTAAGTA CATTAAGTAT |
| 661 | TTTGTTTTAC TGATAGTTTT TTTTTTTTTT TTTTTTTTTT TTTTTGGGTG TGCATGTTTT |
| 721 | GACGTTGATG GCGCGCCTTT TATATGTGTA GTAGGCCTAT TTTCACTAAT GCATGCGATT |
| 781 | GACAATATAA GGCTCACGTA ATAAAATGCT AAAATGCATT TGTAATTGGT AACGTTAGGT |
| 841 | CCACGGGAAA TTTGGCGCCT ATTGCAGCTT TGAATAATCA TTATCATTCC GTGCTCTCAT |
| 901 | TGTGTTTGAA TTCATGCAAA ACACAAGAAA ACCAAGCGAG AAATTTTTTT CCAAACATGT |
| 961 | TGTATTGTCA AAACGGTAAC ACTTTACAAT GAGGTTGATT AGTTCATGTA TTAACTAACA |
| 1021 | TTAAATAACC ATGAGCAATA CATTTGTTAC TGTATCTGTT AATCTTTGTT AACGTTAGTT |
| 1081 | AATAGAAATA CAGATGTTCA TTGTTTGTTC ATGTTAGTTC ACAGTGCATT AACTAATGTT |
| 1141 | AACAAGATAT AAAGTATTAG TAAATGTTGA AATTAACATG TATACGTGCA GTTCATTATT |
| 1201 | AGTTCATGTT AACTAATGTA GTTAACTAAC GAACCTTATT GTAAAAGTGT TACCATCAAA |
| 1261 | ACTAATGTAA TGAAATCAAT TCACCCTGTC ATGTCAGCCT TACAGTCCTG TGTTTTTGTC |
| 1321 | AATATAATCA GAAATAAAAT TAATGTTTGA TTGTCACTAA ATGCTACTGT ATTTCTAAAA |
| 1381 | TCAACAAGTA TTTAACATTA TAAAGTGTGC AATTGGCTGC AAATGTCAGT TTTATTAAAG |
| 1441 | GGTTAGTTCA CCCAAAAATG AAAATAATGT CATTAATGAC TCGCCCTCAT GTCGTTCCAA |
| 1501 | GCCCGTAAGA CCTCCGTTCA TCTTCAGAAC ACAGTTTAAG ATATTTTAGA TTTAGTCCGA |
| 1561 | GAGCTTTCTG TGCCTCCATT GAGAATGTAT GTACGGTATA CTGTCCATGT CCAGAAAGGT |
| 1621 | AATAAAAACA TCAAAGTAGT CCATGTGACA TCAGTGGGTT AGTTAGAATT TTTTGAAGCA |
| 1681 | TCGAATACAT TTTGGTCCAA AAATAACAAA ACCTACGACT TTATTCGGCA TTGTATTCTC |
| 1741 | TTCCGGGTCT GTTGTCAATC CGCGTTCACG ACTTCGCAGT GACGCTACAA TGCTGAATAA |
| 1801 | AGTCGTAGGT TTTGTTATTT TTGGACCAAA ATGTATTTTC GATGCTTCAA ATAATTCTAC |
| 1861 | CTAACCCACT GATGTCACAT GGACTACTTT GATGTTTTTA TTACCTTTCT GGACATGGAC |
| 1921 | AGTATACCGT ACATACATTT TCAGTGGAGG GACAGAAAGC TCTCGGACTA AATCTAAAAT |
| 1981 | ATCTTAAACT GTGTTCCGAA GATGAACGGA GGTGTTACGG GCTTGGAACG ACATGAGGGT |
| 2041 | GAGTCATTAA TGACATCTTT TCATTTTTGG GTGAACTAAC CCTTTAATGC TGTAATCAGA |
| 2101 | GAGTGTATGT GTAATTGTTA CATTTATTGC ATACAATATA AATATTTATT TGTTGTTTTT |
| 2161 | ACAGAGAATG CACCCAAATT ACCTCAAAAA CTACTCTAAA TTGACAGCAC AGAAGAGAAA |
| 2221 | GATCGGGACC TCCACCCATG CTTCCAGCAG TAAGCAACTG AAAGTTGACT CAGTTTTCCC |
| 2281 | AGTCAAACAT GTGTCTCCAG TCACTGTGAA CAAAGCTATA TTAAGGTACA TCATTCAAGG |
| 2341 | ACTTCATCCT TTCAGCACTG TTGATCTGCC ATCATTTAAA GAGCTGATTA GTACACTGCA |
| 2401 | GCCTGGCATT TCTGTCATTA CAAGGCCTAC TTTACGCTCC AAGATAGCTG AAGCTGCTCT |
| 2461 | GATCATGAAA CAGAAAGTGA CTGCTGCCAT GAGTGAAGTT GAATGGATTG CAACCACAAC |
| 2521 | GGATTGTTGG ACTGCACGTA GAAAGTCATT CATTGGTGTA ACTGCTCACT GGATCAACCC |
| 2581 | TGGAAGTCTT GAAAGACATT CCGCTGCACT TGCCTGCAAA AGATTAATGG GCTCTCATAC |
| 2641 | TTTTGAGGTA CTGGCCAGTG CCATGAATGA TATCCACTCA GAGTATGAAA TACGTGACAA |
| 2701 | GGTTGTTTGC ACAACCACAG ACAGTGGTTC CAACTTTATG AAGGCTTTCA GAGTTTTTGG |
| 2761 | TGTGGAAAAC AATGATATCG AGACTGAGGC AAGAAGGTGT GAAAGTGATG ACACTGATTC |
| 2821 | TGAAGGCTGT GGTGAGGGAA GTGATGGTGT GGAATTCCAA GATGCCTCAC GAGTCCTGGA |
| 2881 | CCAAGACGAT GGCTTCGAAT TCCAGCTACC AAAACATCAA AAGTGTGCCT GTCACTTACT |
| 2941 | TAACCTAGTC TCAAGCGTTG ATGCCCAAAA AGCTCTCTCA AATGAACACT ACAAGAAACT |
| 3001 | CTACAGATCT GTCTTTGGCA AATGCCAAGC TTTATGGAAT AAAAGCAGCC GATCGGCTCT |
| 3061 | AGCAGCTGAA GCTGTTGAAT CAGAAAGCCG GCTTCAGCTT TTAAGGCCAA ACCAAACGCG |
| 3121 | GTGGAATTCA ACTTTTATGG CTGTTGACAG AATTCTTCAA ATTTGCAAAG AAGCAGGAGA |
| 3181 | AGGCGCACTT CGGAATATAT GCACCTCTCT TGAGGTTCCA ATGTAAGTGT TTTTCCCCTC |
| 3241 | TATCGATGTA AACAAATGTG GGTTGTTTTT GTTTAATACT CTTTGATTAT GCTGATTTCT |
| 3301 | CCTGTAGGTT TAATCCAGCA GAAATGCTGT TCTTGACAGA GTGGGCCAAC ACAATGCGTC |
| 3361 | CAGTTGCAAA AGTACTCGAC ATCTTGCAAG CGGAAACGAA TACACAGCTG GGGTGGCTGC |
| 3421 | TGCCTAGTGT CCATCAGTTA AGCTTGAAAC TTCAGCGACT CCACCATTCT CTCAGGTACT |
| 3481 | GTGACCCACT TGTGGATGCC CTACAACAAG GAATCCAAAC ACGATTCAAG CATATGTTTG |
| 3541 | AAGATCCTGA GATCATAGCA GCTGCCATCC TTCTCCCTAA ATTTCGGACC TCTTGGACAA |
| 3601 | ATGATGAAAC CATCATAAAA CGAGGTAAAT GAATGCAAGC AACATACACT TGACGAATTC |
| 3661 | TAATCTGGGC AACCTTTGAG CCATACCAAA ATTATTCTTT TATTTATTTA TTTTTGCACT |
| 3721 | TTTTAGGAAT GTTATATCCC ATCTTTGGCT GTGATCTCAA TATGAATATT GATGTAAAGT |
| 3781 | ATTCTTGCAG CAGGTTGTAG TTATCCCTCA GTGTTTCTTG AAACCAAACT CATATGTATC |
| 3841 | ATATGTGGTT TGGAAATGCA GTTAGATTTT ATGCTAAAAT AAGGGATTTG CATGATTTTA |
| 3901 | GATGTAGATG ACTGCACGTA AATGTAGTTA ATGACAAAAT CCATAAAATT TGTTCCCAGT |
| 3961 | CAGAAGCCCC TCAACCAAAC TTTTCTTTGT GTCTGCTCAC TGTGCTTGTA GGCATGGACT |
| 4021 | ACATCAGAGT GCATCTGGAG CCTTTGGACC ACAAGAAGGA ATTGGCCAAC AGTTCATCTG |
| 4081 | ATGATGAAGA TTTTTTCGCT TCTTTGAAAC CGACAACACA TGAAGCCAGC AAAGAGTTGG |
| 4141 | ATGGATATCT GGCCTGTGTT TCAGACACCA GGGAGTCTCT GCTCACGTTT CCTGCTATTT |
| 4201 | GCAGCCTCTC TATCAAGACT AATACACCTC TTCCCGCATC GGCTGCCTGT GAGAGGCTTT |
| 4261 | TCAGCACTGC AGGATTGCTT TTCAGCCCCA AAAGAGCTAG GCTTGACACT AACAATTTTG |
| 4321 | AGAATCAGCT TCTACTGAAG TTAAATCTGA GGTTTTACAA CTTTGAGTAG CGTGTACTGG |
| 4381 | CATTAGATTG TCTGTCTTAT AGTTTGATAA TTAAATACAA ACAGTTCTAA AGCAGGATAA |
| 4441 | AACCTTGTAT GCATTTCATT TAATGTTTTT TGAGATTAAA AGCTTAAACA AGAATCTCTA |
| 4501 | GTTTTCTTTC TTGCTTTTAC TTTTACTTCC TTAATACTCA AGTACAATTT TAATGGAGTA |
| 4561 | CTTTTTTACT TTTACTCAAG TAAGATTCTA GCCAGATACT TTTACTTTTA ATTGAGTAAA |
| 4621 | ATTTTCCCTA AGTACTTGTA CTTTCACTTG AGTAAAATTT TTGAGTACTT TTTACACCTC |
| 4681 | TG. |

Exemplary transposon/transposase systems of the disclosure include, but are not limited to, piggyBac and piggyBac-like transposons and transposases.

PiggyBac and piggyBac-like transposases recognizes transposon-specific inverted terminal repeat sequences (ITRs) on the ends of the transposon, and moves the contents between the ITRs into TTAA or TTAT chromosomal sites. The piggyBac or piggyBac-like transposon system has no payload limit for the genes of interest that can be included between the ITRs.

In certain embodiments, and, in particular, those embodiments wherein the transposon is a piggyBac transposon, the transposase is a piggyBac™, Super piggyBac™ (SPB) transposase. In certain embodiments, and, in particular, those embodiments wherein the transposase is a piggyBac™, Super piggyBac™ (SPB), the sequence encoding the transposase is an mRNA sequence.

In certain embodiments of the methods of the disclosure, the transposase enzyme is a piggyBac or piggyBac-like transposase enzyme.

In certain embodiments of the methods of the disclosure, the transposase enzyme is a piggyBac or a piggyBac-like transposase enzyme. The piggyBac (PB) or piggyBac-like transposase enzyme may comprise or consist of an amino acid sequence at least 5%, 10%, 15%, 20%, 25%, 30%, 35%, 40%, 45%, 50%, 55%, 60%, 65%, 70%, 75%, 80%, 85%, 90%, 95%, 99% or any percentage in between identical to:

| (SEQ ID NO: 14487) | |
|---|---|
| 1 | MGSSLDDEHI LSALLQSDDE LVGEDSDSEI SDHVSEDDVQ SDTEEAFIDE VHEVQPTSSG |
| 61 | SEILDEQNVI EQPGSSLASN RILTLPQRTI RGKNKHCWST SKSTRRSRVS ALNIVRSQRG |
| 121 | PTRMCRNIYD PLLCFKLFFT DEIISEIVKW TNAEISLKRR ESMTGATFRD TNEDEIYAFF |
| 181 | GILVMTAVRK DNHMSTDDLF DRSLSMVYVS VMSRDRFDFL IRCLRMDDKS IRPTLRENDV |
| 241 | FTPVRKIWDL FIHQCIQNYT PGAHLTIDEQ LLGFRGRCPF RMYIPNKPSK YGIKILMMCD |
| 301 | SGTKYMINGM PYLGRGTQTN GVPLGEYYVK ELSKPVHGSC RNITCDNWFT SIPLAKNLLQ |
| 361 | EPYKLTIVGT VRSNKREIPE VLKNSRSRPV GTSMFCFDGP LTLVSYKPKP AKMVYLLSSC |
| 421 | DEDASINEST GKPQMVMYYN QTKGGVDTLD QMCSVMTCSR KTNRWPMALL YGMINIACIN |
| 481 | SFIIYSHNVS SKGEKVQSRK KFMRNLYMSL TSSFMRKRLE APTLKRYLRD NISNILPNEV |
| 541 | PGTSDDSTEE PVMKKRTYCT YCPSKIRRKA NASCKKCKKV ICREHNIDMC QSCF. |

In certain embodiments of the methods of the disclosure, the transposase enzyme is a piggyBac or piggyBac-like transposase enzyme that comprises or consists of an amino acid sequence having an amino acid substitution at one or more of positions 30, 165, 282, or 538 of the sequence:

| (SEQ ID NO: 14487) | |
|---|---|
| 1 | MGSSLDDEHI LSALLQSDDE LVGEDSDSEI SDHVSEDDVQ SDTEEAFIDE VHEVQPTSSG |
| 61 | SEILDEQNVI EQPGSSLASN RILTLPQRTI RGKNKHCWST SKSTRRSRVS ALNIVRSQRG |
| 121 | PTRMCRNIYD PLLCFKLFFT DEIISEIVKW TNAEISLKRR ESMTGATFRD TNEDEIYAFF |
| 181 | GILVMTAVRK DNHMSTDDLF DRSLSMVYVS VMSRDRFDFL IRCLRMDDKS IRPTLRENDV |
| 241 | FTPVRKIWDL FIHQCIQNYT PGAHLTIDEQ LLGFRGRCPF RMYIPNKPSK YGIKILMMCD |
| 301 | SGTKYMINGM PYLGRGTQTN GVPLGEYYVK ELSKPVHGSC RNITCDNWFT SIPLAKNLLQ |
| 361 | EPYKLTIVGT VRSNKREIPE VLKNSRSRPV GTSMFCFDGP LTLVSYKPKP AKMVYLLSSC |
| 421 | DEDASINEST GKPQMVMYYN QTKGGVDTLD QMCSVMTCSR KTNRWPMALL YGMINIACIN |
| 481 | SFIIYSHNVS SKGEKVQSRK KFMRNLYMSL TSSFMRKRLE APTLKRYLRD NISNILPNEV |
| 541 | PGTSDDSTEE PVMKKRTYCT YCPSKIRRKA NASCKKCKKV ICREHNIDMC QSCF. |

In certain embodiments, the transposase enzyme is a piggyBac or piggyBac-like transposase enzyme that comprises or consists of an amino acid sequence having an amino acid substitution at two or more of positions 30, 165, 282, or 538 of the sequence of SEQ ID NO: 14487. In certain embodiments, the transposase enzyme is a piggyBac or piggyBac-like transposase enzyme that comprises or consists of an amino acid sequence having an amino acid substitution at three or more of positions 30, 165, 282, or 538 of the sequence of SEQ ID NO: 14487. In certain embodiments, the transposase enzyme is a piggyBac or piggyBac-like transposase enzyme that comprises or consists of an amino acid sequence having an amino acid substitution at each of the following positions 30, 165, 282, and 538 of the sequence of SEQ ID NO: 14487. In certain embodiments, the amino acid substitution at position 30 of the sequence of SEQ ID NO: 14487 is a substitution of a valine (V) for an isoleucine (I). In certain embodiments, the amino acid substitution at position 165 of the sequence of SEQ ID NO: 14487 is a substitution of a serine (S) for a glycine (G). In certain embodiments, the amino acid substitution at position 282 of the sequence of SEQ ID NO: 14487 is a substitution of a valine (V) for a methionine (M). In certain embodiments, the amino acid substitution at position 538 of the sequence of SEQ ID NO: 14487 is a substitution of a lysine (K) for an asparagine (N).

In certain embodiments of the methods of the disclosure, the transposase enzyme is a Super piggyBac™ (SPB) or piggyBac-like transposase enzyme. In certain embodiments, the Super piggyBac™ (SPB) or piggyBac-like transposase enzyme of the disclosure may comprise or consist of the amino acid sequence of the sequence of SEQ ID NO: 14487 wherein the amino acid substitution at position 30 is a substitution of a valine (V) for an isoleucine (I), the amino acid substitution at position 165 is a substitution of a serine (S) for a glycine (G), the amino acid substitution at position 282 is a substitution of a valine (V) for a methionine (M), and the amino acid substitution at position 538 is a substitution of a lysine (K) for an asparagine (N). In certain embodiments, the Super piggyBac™ (SPB) or piggyBac-like transposase enzyme may comprise or consist of an amino acid sequence at least 5%, 10%, 15%, 20%, 25%, 30%, 35%, 40%, 45%, 50%, 55%, 60%, 65%, 70%, 75%, 80%, 85%, 90%, 95%, 99% or any percentage in between identical to:

| (SEQ ID NO: 14484) | |
|---|---|
| 1 | MGSSLDDEHI LSALLQSDDE LVGEDSDSEV SDHVSEDDVQ SDTEEAFIDE VHEVQPTSSG |
| 61 | SEILDEQNVI EQPGSSLASN RILTLPQRTI RGKNKHCWST SKSTRRSRVS ALNIVRSQRG |
| 121 | PTRMCRNIYD PLLCFKLFFT DEIISEIVKW TNAEISLKRR ESMTSATFRD TNEDEIYAFF |
| 181 | GILVMTAVRK DNHMSTDDLF DRSLSMVYVS VMSRDRFDFL IRCLRMDDKS IRPTLRENDV |
| 241 | FTPVRKIWDL FIHQCIQNYT PGAHLTIDEQ LLGFRGRCPF RVYIPNKPSK YGIKILMMCD |
| 301 | SGTKYMINGM PYLGRGTQTN GVPLGEYYVK ELSKPVHGSC RNITCDNWFT SIPLAKNLLQ |
| 361 | EPYKLTIVGT VRSNKREIPE VLKNSRSRPV GTSMFCFDGP LTLVSYKPKP AKMVYLLSSC |
| 421 | DEDASINEST GKPQMVMYYN QTKGGVDTLD QMCSVMTCSR KTNRWPMALL YGMINIACIN |
| 481 | SFIIYSHNVS SKGEKVQSRK KFMRNLYMSL TSSFMRKRLE APTLKRYLRD NISNILPKEV |
| 541 | PGTSDDSTEE PVMKKRTYCT YCPSKIRRKA NASCKKCKKV ICREHNIDMC QSCF. |

In certain embodiments of the methods of the disclosure, including those embodiments wherein the transposase comprises the above-described mutations at positions 30, 165, 282 and/or 538, the piggyBac™, Super piggyBac™ or piggyBac-like transposase enzyme may further comprise an amino acid substitution at one or more of positions 3, 46, 82, 103, 119, 125, 177, 180, 185, 187, 200, 207, 209, 226, 235, 240, 241, 243, 258, 296, 298, 311, 315, 319, 327, 328, 340, 421, 436, 456, 470, 486, 503, 552, 570 and 591 of the sequence of SEQ ID NO: 14487 or SEQ ID NO: 14484. In certain embodiments, including those embodiments wherein the transposase comprises the above-described mutations at positions 30, 165, 282 and/or 538, the piggyBac™, Super piggyBac™ or piggyBac-like transposase enzyme may further comprise an amino acid substitution at one or more of positions 46, 119, 125, 177, 180, 185, 187, 200, 207, 209, 226, 235, 240, 241, 243, 296, 298, 311, 315, 319, 327, 328, 340, 421, 436, 456, 470, 485, 503, 552 and 570. In certain embodiments, the amino acid substitution at position 3 of SEQ ID NO: 14487 or SEQ ID NO: 14484 is a substitution of an asparagine (N) for a serine (S). In certain embodiments, the amino acid substitution at position 46 of SEQ ID NO: 14487 or SEQ ID NO: 14484 is a substitution of a serine (S) for an alanine (A). In certain embodiments, the amino acid substitution at position 46 of SEQ ID NO: 14487 or SEQ ID NO: 14484 is a substitution of a threonine (T) for an alanine (A). In certain embodiments, the amino acid substitution at position 82 of SEQ ID NO: 14487 or SEQ ID NO: 14484 is a substitution of a tryptophan (W) for an isoleucine (I). In certain embodiments, the amino acid substitution at position 103 of SEQ ID NO: 14487 or SEQ ID NO: 14484 is a substitution of a proline (P) for a serine (S). In certain embodiments, the amino acid substitution at position 119 of SEQ ID NO: 14487 or SEQ ID NO: 14484 is a substitution of a proline (P) for an arginine (R). In certain embodiments, the amino acid substitution at position 125 of SEQ ID NO: 14487 or SEQ ID NO: 14484 is a substitution of an alanine (A) a cysteine (C). In certain embodiments, the amino acid substitution at position 125 of SEQ ID NO: 14487 or SEQ ID NO: 14484 is a substitution of a leucine (L) for a cysteine (C). In certain embodiments, the amino acid substitution at position 177 of SEQ ID NO: 14487 or SEQ ID NO: 14484 is a substitution of a lysine (K) for a tyrosine (Y). In certain embodiments, the amino acid substitution at position 177 of SEQ ID NO: 14487 or SEQ ID NO: 14484 is a substitution of a histidine (H) for a tyrosine (Y). In certain embodiments, the amino acid substitution at position 180 of SEQ ID NO: 14487 or SEQ ID NO: 14484 is a substitution of a leucine (L) for a phenylalanine (F). In certain embodiments, the amino acid substitution at position 180 of SEQ ID NO: 14487 or SEQ ID NO: 14484 is a substitution of an isoleucine (I) for a phenylalanine (F). In certain embodiments, the amino acid substitution at position 180 of SEQ ID NO: 14487 or SEQ ID NO: 14484 is a substitution of a valine (V) for a phenylalanine (F). In certain embodiments, the amino acid substitution at position 185 of SEQ ID NO: 14487 or SEQ ID NO: 14484 is a substitution of a leucine (L) for a methionine (M). In certain embodiments, the amino acid substitution at position 187 of SEQ ID NO: 14487 or SEQ ID NO: 14484 is a substitution of a glycine (G) for an alanine (A). In certain embodiments, the amino acid substitution at position 200 of SEQ ID NO: 14487 or SEQ ID NO: 14484 is a substitution of a tryptophan (W) for a phenylalanine (F). In certain embodiments, the amino acid substitution at position 207 of SEQ ID NO: 14487 or SEQ ID NO: 14484 is a substitution of a proline (P) for a valine (V). In certain embodiments, the amino acid substitution at position 209 of SEQ ID NO: 14487 or SEQ ID NO: 14484 is a substitution of a phenylalanine (F) for a valine (V). In certain embodiments, the amino acid substitution at position 226 of SEQ ID NO: 14487 or SEQ ID NO: 14484 is a substitution of a phenylalanine (F) for a methionine (M). In certain embodiments, the amino acid substitution at position 235 of SEQ ID NO: 14487 or SEQ ID NO: 14484 is a substitution of an arginine (R) for a leucine (L). In certain embodiments, the amino acid substitution at position 240 of SEQ ID NO: 14487 or SEQ ID NO: 14484 is a substitution of a lysine (K) for a valine (V). In certain embodiments, the amino acid substitution at position 241 of SEQ ID NO: 14487 or SEQ ID NO: 14484 is a substitution of a leucine (L) for a phenylalanine (F). In certain embodiments, the amino acid substitution at position 243 of SEQ ID NO: 14487 or SEQ ID NO: 14484 is a substitution of a lysine (K) for a proline (P). In certain embodiments, the amino acid substitution at position 258 of SEQ ID NO: 14487 or SEQ ID NO: 14484 is a substitution of a serine (S) for an asparagine (N). In certain embodiments, the amino acid substitution at position 296 of SEQ ID NO: 14487 or SEQ ID NO: 14484 is a substitution of a tryptophan (W) for a leucine (L). In certain embodiments, the amino acid substitution at position 296 of SEQ ID NO: 14487 or SEQ ID NO: 14484 is a substitution of a tyrosine (Y) for a leucine (L). In certain embodiments, the amino acid substitution at position 296 of SEQ ID NO: 14487 or SEQ ID NO: 14484 is a substitution of a phenylalanine (F) for a leucine (L). In certain embodiments, the amino acid substitution at position 298 of SEQ ID NO: 14487 or SEQ ID NO: 14484 is a substitution of a leucine (L) for a methionine (M). In certain embodiments, the amino acid substitution at position 298 of SEQ ID NO: 14487 or SEQ ID NO: 14484 is a substitution of an alanine (A) for a methionine (M). In certain embodiments, the amino acid substitution at position 298 of SEQ ID NO: 14487 or SEQ ID NO: 14484 is a substitution of a valine (V) for a methionine (M). In certain embodiments, the amino acid substitution at position 311 of SEQ ID NO: 14487 or SEQ ID NO: 14484 is a substitution of an isoleucine (I) for a proline (P). In certain embodiments, the amino acid substitution at position 311 of SEQ ID NO: 14487 or SEQ ID NO: 14484 is a substitution of a valine for a proline (P). In certain embodiments, the amino acid substitution at position 315 of SEQ ID NO: 14487 or SEQ ID NO: 14484 is a substitution of a lysine (K) for an arginine (R). In certain embodiments, the amino acid substitution at position 319 of SEQ ID NO: 14487 or SEQ ID NO: 14484 is a substitution of a glycine (G) for a threonine (T). In certain embodiments, the amino acid substitution at position 327 of SEQ ID NO: 14487 or SEQ ID NO: 14484 is a substitution of an arginine (R) for a tyrosine (Y). In certain embodiments, the amino acid substitution at position 328 of SEQ ID NO: 14487 or SEQ ID NO: 14484 is a substitution of a valine (V) for a tyrosine (Y). In certain embodiments, the amino acid substitution at position 340 of SEQ ID NO: 14487 or SEQ ID NO: 14484 is a substitution of a glycine (G) for a cysteine (C). In certain embodiments, the amino acid substitution at position 340 of SEQ ID NO: 14487 or SEQ ID NO: 14484 is a substitution of a leucine (L) for a cysteine (C). In certain embodiments, the amino acid substitution at position 421 of SEQ ID NO: 14487 or SEQ ID NO: 14484 is a substitution of a histidine (H) for the aspartic acid (D). In certain embodiments, the amino acid substitution at position 436 of SEQ ID NO: 14487 or SEQ ID NO: 14484 is a substitution of an isoleucine (I) for a valine (V). In certain embodiments, the amino acid substitution at position 456 of SEQ ID NO: 14487 or SEQ ID NO: 14484 is a substitution of a tyrosine (Y) for a methionine (M). In certain embodiments, the amino acid substitution at position 470 of SEQ ID NO: 14487 or SEQ ID NO: 14484 is a substitution of a phenylalanine (F) for a leucine (L). In certain embodiments, the amino acid substitution at position 485 of SEQ ID NO: 14487 or SEQ ID NO: 14484 is a substitution of a lysine (K) for a serine (S). In certain embodiments, the amino acid substitution at position 503 of SEQ ID NO: 14487 or SEQ ID NO: 14484 is a substitution of a leucine (L) for a methionine (M). In certain embodiments, the amino acid substitution at position 503 of SEQ ID NO: 14487 or SEQ ID NO: 14484 is a substitution of an isoleucine (I) for a methionine (M). In certain embodiments, the amino acid substitution at position 552 of SEQ ID NO: 14487 or SEQ ID NO: 14484 is a substitution of a lysine (K) for a valine (V). In certain embodiments, the amino acid substitution at position 570 of SEQ ID NO: 14487 or SEQ ID NO: 14484 is a substitution of a threonine (T) for an alanine (A). In certain embodiments, the amino acid substitution at position 591 of SEQ ID NO: 14487 or SEQ ID NO: 14484 is a substitution of a proline (P) for a glutamine (Q). In certain embodiments, the amino acid substitution at position 591 of SEQ ID NO: 14487 or SEQ ID NO: 14484 is a substitution of an arginine (R) for a glutamine (Q).

In certain embodiments of the methods of the disclosure, including those embodiments wherein the transposase comprises the above-described mutations at positions 30, 165, 282 and/or 538, the piggyBac™ or piggyBac-like transposase enzyme or may comprise or the Super piggyBac™ transposase enzyme may further comprise an amino acid substitution at one or more of positions 103, 194, 372, 375, 450, 509 and 570 of the sequence of SEQ ID NO: 14487 or SEQ ID NO: 14484. In certain embodiments of the methods of the disclosure, including those embodiments wherein the transposase comprises the above-described mutations at positions 30, 165, 282 and/or 538, the piggyBac™ or piggyBac-like transposase enzyme may comprise or the Super piggyBac™ transposase enzyme may further comprise an amino acid substitution at two, three, four, five, six or more of positions 103, 194, 372, 375, 450, 509 and 570 of the sequence of SEQ ID NO: 14487 or SEQ ID NO: 14484. In certain embodiments, including those embodiments wherein the transposase comprises the above-described mutations at positions 30, 165, 282 and/or 538, the piggyBac™ or piggyBac-like transposase enzyme may comprise or the Super piggyBac™ transposase enzyme may further comprise an amino acid substitution at positions 103, 194, 372, 375, 450, 509 and 570 of the sequence of SEQ ID NO: 14487 or SEQ ID NO: 14484. In certain embodiments, the amino acid substitution at position 103 of SEQ ID NO: 14487 or SEQ ID NO: 14484 is a substitution of a proline (P) for a serine (S). In certain embodiments, the amino acid substitution at position 194 of SEQ ID NO: 14487 or SEQ ID NO: 14484 is a substitution of a valine (V) for a methionine (M). In certain embodiments, the amino acid substitution at position 372 of SEQ ID NO: 14487 or SEQ ID NO: 14484 is a substitution of an alanine (A) for an arginine (R). In certain embodiments, the amino acid substitution at position 375 of SEQ ID NO: 14487 or SEQ ID NO: 14484 is a substitution of an alanine (A) for a lysine (K). In certain embodiments, the amino acid substitution at position 450 of SEQ ID NO: 14487 or SEQ ID NO: 14484 is a substitution of an asparagine (N) for an aspartic acid (D). In certain embodiments, the amino acid substitution at position 509 of SEQ ID NO: 14487 or SEQ ID NO: 14484 is a substitution of a glycine (G) for a serine (S). In certain embodiments, the amino acid substitution at position 570 of SEQ ID NO: 14487 or SEQ ID NO: 14484 is a substitution of a serine (S) for an asparagine (N). In certain embodiments, the piggyBac™ or piggyBac-like transposase enzyme may comprise a substitution of a valine (V) for a methionine (M) at position 194 of SEQ ID NO: 14487. In certain embodiments, including those embodiments wherein the piggyBac™ or piggyBac-like transposase enzyme may comprise a substitution of a valine (V) for a methionine (M) at position 194 of SEQ ID NO: 14487, the piggyBac™ or piggyBac-like transposase enzyme may further comprise an amino acid substitution at positions 372, 375 and 450 of the sequence of SEQ ID NO: 14487 or SEQ ID NO: 14484. In certain embodiments, the piggyBac™ or piggyBac-like transposase enzyme may comprise a substitution of a valine (V) for a methionine (M) at position 194 of SEQ ID NO: 14487, a substitution of an alanine (A) for an arginine (R) at position 372 of SEQ ID NO: 14487, and a substitution of an alanine (A) for a lysine (K) at position 375 of SEQ ID NO: 14487. In certain embodiments, the piggyBac™ or piggyBac-like transposase enzyme may comprise a substitution of a valine (V) for a methionine (M) at position 194 of SEQ ID NO: 14487, a substitution of an alanine (A) for an arginine (R) at position 372 of SEQ ID NO: 14487, a substitution of an alanine (A) for a lysine (K) at position 375 of SEQ ID NO: 14487 and a substitution of an asparagine (N) for an aspartic acid (D) at position 450 of SEQ ID NO: 14487.

In certain embodiments, the piggyBac or piggyBac-like transposase enzyme is isolated or derived from an insect. In certain embodiments, the insect is Trichoplusia ni (GenBank Accession No. AAA87375; SEQ ID NO: 14666), Argyrogramma agnata (GenBank Accession No. GU477713; SEQ ID NO: 14534, SEQ ID NO: 14667), Anopheles gambiae (GenBank Accession No. XP 312615 (SEQ ID NO: 14668); GenBank Accession No. XP 320414 (SEQ ID NO: 14669); GenBank Accession No. XP 310729 (SEQ ID NO: 14670)), Aphis gossypii (GenBank Accession No. GU329918; SEQ ID NO: 14671, SEQ ID NO: 14672), Acyrthosiphon pisum (GenBank Accession No. XP 001948139; SEQ ID NO: 14673), Agrotis ipsilon (GenBank Accession No. GU477714; SEQ ID NO: 14537, SEQ ID NO: 14674), Bombyx mori (GenBank Accession No. BAD11135; SEQ ID NO: 14505), Chilo suppressalis (GenBank Accession No. JX294476; SEQ ID NO: 14675, SEQ ID NO: 14676), Drosophila melanogaster (GenBank Accession No. AAL39784; SEQ ID NO: 14677), Helicoverpa armigera (GenBank Accession No. ABS18391; SEQ ID NO: 14525), Heliothis virescens (GenBank Accession No. ABD76335; SEQ ID NO: 14678), Macdunnoughia crassisigna (GenBank Accession No. EU287451; SEQ ID NO: 14679, SEQ ID NO: 14680), Pectinophora gossypiella (GenBank Accession No. GU270322; SEQ ID NO: 14530, SEQ ID NO: 14681), Tribolium castaneum (GenBank Accession No. XP 001814566; SEQ ID NO: 14682), Ctenoplusia agnata (also called Argyrogramma agnata), Messour bouvieri, Megachile rotundata, Bombus impatiens, Mamestra brassicae, Mayetiola destructor or Apis mellifera.

In certain embodiments, the piggyBac or piggyBac-like transposase enzyme is isolated or derived from an insect. In certain embodiments, the insect is Trichoplusia ni (AAA87375).

In certain embodiments, the piggyBac or piggyBac-like transposase enzyme is isolated or derived from an insect. In certain embodiments, the insect is Bombyx mori (BAD11135).

In certain embodiments, the piggyBac or piggyBac-like transposase enzyme is isolated or derived from a crustacean. In certain embodiments, the crustacean is Daphnia pulicaria (AAM76342, SEQ ID NO: 14683).

In certain embodiments, the piggyBac or piggyBac-like transposase enzyme is isolated or derived from a vertebrate. In certain embodiments, the vertebrate is Xenopus tropicalis (GenBank Accession No. BAF82026; SEQ ID NO: 14518), Homo sapiens (GenBank Accession No. NP 689808; SEQ ID NO: 14684), Mus musculus (GenBank Accession No. NP 741958; SEQ ID NO: 14685), Macaca fascicularis (GenBank Accession No. AB179012; SEQ ID NO: 14686, SEQ ID NO: 14687), Rattus norvegicus (GenBank Accession No. XP 220453; SEQ ID NO: 14688) or Myotis lucifugus.

In certain embodiments, the piggyBac or piggyBac-like transposase enzyme is isolated or derived from a urochordate. In certain embodiments, the urochordate is Ciona intestinalis (GenBank Accession No. XP 002123602; SEQ ID NO: 14689).

In certain embodiments, the piggyBac or piggyBac-like transposase inserts a transposon at the sequence 5′-TTAT-3′ within a chromosomal site (a TTAT target sequence).

In certain embodiments, the piggyBac or piggyBac-like transposase inserts a transposon at the sequence 5′-TTAA-3′ within a chromosomal site (a TTAA target sequence).

In certain embodiments, the target sequence of the piggyBac or piggyBac-like transposon comprises or consists of 5′-CTAA-3′, 5′-TTAG-3′, 5′-ATAA-3′, 5′-TCAA-3′, 5′AGTT-3′, 5′-ATTA-3′, 5′-GTTA-3′, 5′-TTGA-3′, 5′-TTTA-3′, 5′-TTAC-3′, 5′-ACTA-3′, 5′-AGGG-3′, 5′-CTAG-3′, 5′-TGAA-3′, 5′-AGGT-3′, 5′-ATCA-3′, 5′-CTCC-3′, 5′-TAAA-3′, 5′-TCTC-3′, 5′TGAA-3′, 5′-AAAT-3′, 5′-AATC-3′, 5′-ACAA-3′, 5′-ACAT-3′, 5′-ACTC-3′, 5′-AGTG-3′, 5′-ATAG-3′, 5′-CAAA-3′, 5′-CACA-3′, 5′-CATA-3′, 5′-CCAG-3′, 5′-CCCA-3′, 5′-CGTA-3′, 5′-GTCC-3′, 5′-TAAG-3′, 5′-TCTA-3′, 5′-TGAG-3′, 5′-TGTT-3′, 5′-TTCA-3′5′-TTCT-3′ and 5′-TTTT-3′.

In certain embodiments of the methods of the disclosure, the transposase enzyme is a piggyBac or piggyBac-like transposase enzyme. In certain embodiments, the piggyBac or piggyBac-like transposase enzyme is isolated or derived from Bombyx mori. The piggyBac or piggyBac-like transposase enzyme may comprise or consist of an amino acid sequence at least 5%, 10%, 15%, 20%, 25%, 30%, 35%, 40%, 45%, 50%, 55%, 60%, 65%, 70%, 75%, 80%, 85%, 90%, 95%, 99% or any percentage in between identical to:

| (SEQ ID NO: 14504) | |
|---|---|
| 1 | MDIERQEERI RAMLEEELSD YSDESSSEDE TDHCSEHEVN YDTEEERIDS VDVPSNSRQE |
| 61 | EANAIIANES DSDPDDDLPL SLVRQRASAS RQVSGPFYTS KDGTKWYKNC QRPNVRLRSE |
| 121 | NIVTEQAQVK NIARDASTEY ECWNIFVTSD MLQEILTHTN SSIRHRQTKT AAENSSAETS |
| 181 | FYMQETTLCE LKALIALLYL AGLIKSNRQS LKDLWRTDGT GVDIFRTTMS LQRFQFLQNN |
| 241 | IRFDDKSTRD ERKQTDNMAA FRSIFDQFVQ CCQNAYSPSE FLTIDEMLLS FRGRCLFRVY |
| 301 | IPNKPAKYGI KILALVDAKN FDVVNLEVYA GKQPSGPYAV SNRPFEVVER LIQPVARSHR |
| 361 | NVTFDNWFTG YELMLHLLNE YRLTSVGTVR KNKRQIPESF IRTDRQPNSS VFGFQKDITL |
| 421 | VSYAPKKNKV VVVMSTMHHD NSIDESTGEK QKPEMITFYN STKAGVDVVD ELSANYNVSR |
| 481 | NSKRWPMTLF YGVLNMAAIN ACIIYRANKN VTIKRTEFIR SLGLSMIYEH LHSRNKKKNI |
| 541 | PTYLRQRIEK QLGEPSPRHV NVPGRYVRCQ DCPYKKDRKT KHSCNACAKP ICMEHAKFLC |
| 601 | ENCAELDSSL. |

The piggyBac (PB) or piggyBac-like transposase enzyme may comprise or consist of an amino acid sequence at least 5%, 10%, 15%, 20%, 25%, 30%, 35%, 40%, 45%, 50%, 55%, 60%, 65%, 70%, 75%, 80%, 85%, 90%, 95%, 99% or any percentage in between identical to:

| (SEQ ID NO: 14505) | |
|---|---|
| 1 | MDIERQEERI RAMLEEELSD YSDESSSEDE TDHCSEHEVN YDTEEERIDS VDVPSNSRQE |
| 61 | EANAIIANES DSDPDDDLPL SLVRQRASAS RQVSGPFYTS KDGTKWYKNC QRPNVRLRSE |
| 121 | NIVTEQAQVK NIARDASTEY ECWNIFVTSD MLQEILTHTN SSIRHRQTKT AAENSSAETS |
| 181 | FYMQETTLCE LKALIALLYL AGLIKSNRQS LKDLWRTDGT GVDIFRTTMS LQRFQFLQNN |
| 241 | IRFDDKSTRD ERKQTDNMAA FRSIFDQFVQ CCQNAYSPSE FLTIDEMLLS FRGRCLFRVY |
| 301 | IPNKPAKYGI KILALVDAKN FYVVNLEVYA GKQPSGPYAV SNRPFEVVER LIQPVARSHR |
| 361 | NVTFDNWFTG YELMLHLLNE YRLTSVGTVR KNKRQIPESF IRTDRQPNSS VFGFQKDITL |
| 421 | VSYAPKKNKV VVVMSTMHHD NSIDESTGEK QKPEMITFYN STKAGVDVVD ELCANYNVSR |
| 481 | NSKRWPMTLF YGVLNMAAIN ACIIYRTNKN VTIKRTEFIR SLGLSMIYEH LHSRNKKKNI |
| 541 | PTYLRQRIEK QLGEPSPRHV NVPGRYVRCQ DCPYKKDRKT KRSCNACAKP ICMEHAKFLC |
| 601 | ENCAELDSSL. |

In certain embodiments, the piggyBac or piggyBac-like transposase is fused to a nuclear localization signal. In certain embodiments, the amino acid sequence of the piggyBac or piggyBac-like transposase fused to a nuclear localization signal is encoded by a polynucleotide sequence comprising:

| (SEQ ID NO: 14629) | |
|---|---|
| 1 | atggcaccca aaaagaaacg taaagtgatg gacattgaaa gacaggaaga aagaatcagg |
| 61 | gcgatgctcg aagaagaact gagcgactac tccgacgaat cgtcatcaga ggatgaaacc |
| 121 | gaccactgta gcgagcatga ggttaactac gacaccgagg aggagagaat cgactctgtg |
| 181 | gatgtgccct ccaactcacg ccaagaagag gccaatgcaa ttatcgcaaa cgaatcggac |
| 241 | agcgatccag acgatgatct gccactgtcc ctcgtgcgcc agcgggccag cgcttcgaga |
| 301 | caagtgtcag gtccattcta cacttcgaag gacggcacta agtggtacaa gaattgccag |
| 361 | cgacctaacg tcagactccg ctccgagaat atcgtgaccg aacaggctca ggtcaagaat |
| 421 | atcgcccgcg acgcctcgac tgagtacgag tgttggaata tcttcgtgac ttcggacatg |
| 481 | ctgcaagaaa ttctgacgca caccaacagc tcgattaggc atcgccagac caagactgca |
| 541 | gcggagaact catcggccga aacctccttc tatatgcaag agactactct gtgcgaactg |
| 601 | aaggcgctga ttgcactgct gtacttggcc ggcctcatca aatcaaatag gcagagcctc |
| 661 | aaagatctct ggagaacgga tggaactgga gtggatatct ttcggacgac tatgagcttg |
| 721 | cagcggttcc agtttctgca aaacaatatc agattcgacg acaagtccac ccgggacgaa |
| 781 | aggaaacaga ctgacaacat ggctgcgttc cggtcaatat tcgatcagtt tgtgcagtgc |
| 841 | tgccaaaacg cttatagccc atcggaattc ctgaccatcg acgaaatgct tctctccttc |
| 901 | cgggggcgct gcctgttccg agtgtacatc ccgaacaagc cggctaaata cggaatcaaa |
| 961 | atcctggccc tggtggacgc caagaatttc tacgtcgtga atctcgaagt gtacgcagga |
| 1021 | aagcaaccgt cgggaccgta cgctgtttcg aaccgcccgt ttgaagtcgt cgagcggctt |
| 1081 | attcagccgg tggccagatc ccaccgcaat gttaccttcg acaattggtt caccggctac |
| 1141 | gagctgatgc ttcaccttct gaacgagtac cggctcacta gcgtggggac tgtcaggaag |
| 1201 | aacaagcggc agatcccaga atccttcatc cgcaccgacc gccagcctaa ctcgtccgtg |
| 1261 | ttcggatttc aaaaggatat cacgcttgtc tcgtacgccc ccaagaaaaa caaggtcgtg |
| 1321 | gtcgtgatga gcaccatgca tcacgacaac agcatcgacg agtcaaccgg agaaaagcaa |
| 1381 | aagcccgaga tgatcacctt ctacaattca actaaggccg gcgtcgacgt cgtggatgaa |
| 1441 | ctgtgcgcga actataacgt gtcccggaac tctaagcggt ggcctatgac tctcttctac |
| 1501 | ggagtgctga atatggccgc aatcaacgcg tgcatcatct accgcaccaa caagaacgtg |
| 1561 | accatcaagc gcaccgagtt catcagatcg ctgggtttga gcatgatcta cgagcacctc |
| 1621 | cattcacgga acaagaagaa gaatatccct acttacctga ggcagcgtat cgagaagcag |
| 1681 | ttgggagaac caagcccgcg ccacgtgaac gtgccggggc gctacgtgcg gtgccaagat |
| 1741 | tgcccgtaca aaaaggaccg caaaaccaaa agatcgtgta acgcgtgcgc caaacctatc |
| 1801 | tgcatggagc atgccaaatt tctgtgtgaa aattgtgctg aactcgattc ctccctg. |

In certain embodiments, the piggyBac or piggyBac-like transposase is hyperactive. A hyperactive piggyBac or piggyBac-like transposase is a transposase that is more active than the naturally occurring variant from which it is derived. In certain embodiments, the hyperactive piggyBac or piggyBac-like transposase enzyme is isolated or derived from Bombyx mori. In certain embodiments, the piggyBac or piggyBac-like transposase is a hyperactive variant of SEQ ID NO: 14505. In certain embodiments, the hyperactive piggyBac or piggyBac-like transposase comprises a sequence that is at least 90% identical to:

| (SEQ ID NO: 14576) | |
|---|---|
| 1 | MDIERQEERI RAMLEEELSD YSDESSSEDE TDHCSEHEVN YDTEEERIDS VDVPSNSRQE |
| 61 | EANAIIANES DSDPDDDLPL SLVRQRASAS RQMSGPHYTS KDGTKWYKNC QRPNVRLRSE |
| 121 | NIVTEQAQVK NIARDASTEY ECWNIFVTSD MLQEILTHTN SSIRWRQTKT AAENSSASTS |
| 181 | FYMQETTLCE LKALIGLLYI AGLIKSNRQS LKDLWRTDGT GVDIFRTTMS LQRFQFLQNN |
| 241 | IRFDDKSTRD ERKQTDNMAA FRSIFDQFVQ SCQNAYSPSE FLTIDEMLLS FRGRCLFRVY |
| 301 | IPNKPAKYGI KILALVDAKN FYVKNLEVYA GKQPSGPYAV SNRPFEVVER LIQPVARSHR |
| 361 | NVTFDNWFTG YELMLHLLNE YRLTSVGTVR KNKRQIPESF IRTDRQPNSS VFGFQKDITL |
| 421 | VSYAPKKNKV VVVMSTMHHD NSIDESTGEK QKPEMITFYN STKAGVDVVD ELCANYNVSR |
| 481 | NSKRWPMTLF YGVLNMAAIN ACIIYRTNKN VTIKRTEFIR SLGLSMIYEH LHSRNKKKNI |
| 541 | PTYLRQRIEK QLGEPSPRHV NVPGRYVRCQ DCPYKKDRKT KRSCNACAKP ICMEHAKFLC |
| 601 | ENCAELDSHL. |

In certain embodiments, the hyperactive piggyBac or piggyBac-like transposase comprises SEQ ID NO: 14576. In certain embodiments, the hyperactive piggyBac or piggyBac-like transposase comprises a sequence of:

| (SEQ ID NO: 14630) | |
|---|---|
| 1 | MDIERQEERI RAMLEEELSD YSDESSSEDE TDHCSEHEVN YDTEEERIDS VDVPSNSRQE |
| 61 | EANAIIANES DSDPDDDLPL SLVRQRASAS RQVSGPFYTS KDGTKWYKNC QRPNVRLRSE |
| 121 | NIVTEQAQVK NIARDASTEY ECWNIFVTSD MLQEILTHTN SSIRWRQTKT AAENSSAETS |
| 181 | FYMQETTLCE LKALIGLLYI AGLIKSNRQS LKDLWRTDGT GVDIFRTTMS LQRFQFLLNN |
| 241 | IRFDDKSTRD ERKQTDNMAA FRSIFDQFVQ SCQNAYSPSE FLTIDEMLLS FRGRCLFRVY |
| 301 | IPNKPAKYGI KILALVDAKN FYVHNLEVYA GKQPSGPYAV SNRPFEVVER LIQPVARSHR |
| 361 | NVTFDNWFTG YEVMLHLLNE YRLTSVGTVR KNKRQIPESF IRTDRQPNSS VFGFQKDITL |
| 421 | VSYAPKKNKV VVVMSTMHHD NSIDESTGEK QKPEMITFYN STKAGVDVVD ELCANYNVSR |
| 481 | NSKRWPMTLF YGVLNMAAIN ACIIYRTNKN VTIKRTEFIR SLGLSMIYEH LHSRNKKKNI |
| 541 | PTYLRQRIEK QLGEPSPRHV NVPGRYVRCQ DCPYKKDRKT KRSCNACAKP ICMEHAKFLC |
| 601 | ENCAHLDS. |

In certain embodiments, the hyperactive piggyBac or piggyBac-like transposase comprises a sequence of:

| (SEQ ID NO: 14631) | |
|---|---|
| 1 | MDIERQEERI RAMLEEELSD YSDESSSEDE TDHCSEHEVN YDTEEERIDS VDVPSNSRQE |
| 61 | EANAIIANES DSDPDDDLPL SLVRQRASAS RQVSGPFYTS KDGTKWYKNC QRPNVRLRSE |
| 121 | NIVTEQAQVK NIARDASTEY ECWNIFVTSD MLQEILTHTN SSIRWRQTKT AAENSSASTS |
| 181 | FYMQETTLCE LKALIGLLYI AGLIKSNRQS LKDLWRTDGT GVDIFRTTMS LQRFQFLLNN |
| 241 | IRFDDKSTRD ERKQTDNMAA FRSIFDQFVQ SCQNAYSPSE FLTIDEMLLS FRGRCLFRVY |
| 301 | IPNKPAKYGI KILALVDAKN FYVKNLEVYA GKQPSGPYAV SNRPFEVVER LIQPVARSHR |
| 361 | NVTFDNWFTG YELMLHLLNE YRLTSVGTVR KNKRQIPESF IRTDRQPNSS VFGFQKDITL |
| 421 | VSYAPKKNKV VVVMSTMHHD NSIDESTGEK QKPEMITFYN STKAGVDVVD ELCANYNVSR |
| 481 | NSKRWPMTLF YGVLNMAAIN ACIIYRTNKN VTIKRTEFIR SLGLSMIYEH LHSRNKKKNI |
| 541 | PTYLRQRIAM QLGEPSPRHV NVPGRYVRCQ DCPYKKDRKT KRSCNACAKP ICMEHAKFLC |
| 601 | ENCAELDSSL. |

In certain embodiments, the hyperactive piggyBac or piggyBac-like transposase comprises a sequence of:

| (SEQ ID NO: 14632) | |
|---|---|
| 1 | MDIERQEERI RAMLEEELSD YSDESSSEDE TDHCSEHEVN YDTEEERIDS VDVPSNSRQE |
| 61 | EANAIIANES DSDPDDDLPL SLVRQRASAS RQVSGPFYTS KDGTKWYKNC QRPNVRLRSE |
| 121 | NIVTEQAQVK NIARDASTEY ECWNIFVTSD MLQEILTHTN SSIRWRQTKT AAENSSAETS |
| 181 | FYMQETTLCE LKALIGLLYI AGLIKSNRQS LKDLWRTDGT GVDIFRTTMS LQRFQFLLNN |
| 241 | IRFDDKSTRD ERKQTDNMAA FRSIFDQFVQ SCQNAYSPSE FLTIDEMLLS FRGRCLFRVY |
| 301 | IPNKPAKYGI KILALVDAKN FYVKNLEVYA GKQPSGPYAV SNRPFEVVER LIQPVARSHR |
| 361 | NVTFDNWFTG YELMLHLLNE YRLTSVGTVR KNKTQIPENF IRTDRQPNSS VFGFQKDITL |
| 421 | VSYAPKKNKV VVVMSTMHHD NSIDESTGEK QKPEMITFYN STKAGVDVVD ELQANYNVSR |
| 481 | NSKRWPMTLF YGVLNMAAIN ACIIYRTNKN VTIKRTEFIR SLGLSMIYEH LHSRNKKKNI |
| 541 | PTYLRQRIEK QLGEPSPRHV NVPGRYVRCQ DCPYKKDRKT KRSCNACAKP ICMEHAKFLC |
| 601 | ENCAELDSSL. |

In certain embodiments, the hyperactive piggyBac or piggyBac-like transposase comprises a sequence of:

| (SEQ ID NO: 14633) | |
|---|---|
| 1 | MDIERQEERI RAMLEEELSD YSDESSSEDE TDHCSEHEVN YDTEEERIDS VDVPSNSRQE |
| 61 | EANAIIANES DSDPDDDLPL SLVRQRASAS RQVSGPFYTS KDGTKWYKNC QRPNVRLRSE |
| 121 | NIVTEQAQVK NIARDASTEY ECWNIFVTSD MLQEILTHTN SSIRWRQTKT AAENSSAETS |
| 181 | FYMQETTLCE LKALIGLLYI AGLIKSNRQS LKDLWRTDGT GVDIFRTTMS LQRFQFLQNN |
| 241 | IRFDDKSTRD ERKQTDNMAA FRSIFDQFVQ SCQNAYSPSE FLTIDEMLLS FRGRCLFRVY |
| 301 | IPNKPAKYGI KILALVDAKN FYVKNLEVYA GKQPSGPYAV SNRPFEVVER LIQPVARSHR |
| 361 | NVTFDNWFTG YELMLHLLNE YRLTSVGTVR KNKRQIPESF IRTDRQPNSS VFGFQKDITL |
| 421 | VSYAPKKNKV VVVMSTMHHD NSIDESTGEK QKPEMITFYN STKAGVDVVD ELCANYNVSR |
| 481 | NSKRWPMTLF YGVLNMAAIN ACIIYRTNKN VTIKRTEFIR SLGLSMIYEH LHSRNKKKNI |
| 541 | PTYLRQRIEK QLGEPSPRHV NVPGRYVRCQ DCPYKKDRKT KRSCNACAKP ICMEHAKFLC |
| 601 | ENCAELDSSL. |

In certain embodiments, the hyperactive piggyBac or piggyBac-like transposase comprises a sequence of:

| (SEQ ID NO: 14634) | |
|---|---|
| 1 | MDIERQEERI RAMLEEELSD YSDESSSEDE TDHCSEHEVN YDTEEERIDS VDVPSNSRQE |
| 61 | EANAIIANES DSDPDDDLPL SLVRQRASAS RQVSGPFYTS KDGTKWYKNC QRPNVRLRSE |
| 121 | NIVTEQAQVK NIARDASTEY ECWNIFVTSD MLQEILTHTN SSIRHRQTKT AAENSSAETS |
| 181 | FYMQETTLCE LKALIALLYL AGLIKSNRQS LKDLWRTDGT GVDIFRTTMS LQRFQFLQNN |
| 241 | IRFDDKSTRD ERKQTDNMAA FRSIFDQFVQ CCQNAYSPSE FLTIDEMLLS FRGRCLFRVY |
| 301 | IPNKPAKYGI KILALVDAKN DYVVNLEVYA GKQPSGPYAV SNRPFEVVER LIQPVARSHR |
| 361 | NVTFDNWFTG YELMLHLLNE YRLTSVGTVR KNKRQIPESF IRTDRQPNSS VFGFQKDITL |
| 421 | VSYAPKKNKV VVVMSTMHHD NSIDESTGEK QKPEMITFYN STKAGVDVVD ELCANYNVSR |
| 481 | NSKRWPMTLF YGVLNMAAIN ACIIYRTNKN VTIKRTEFIR SLGLSMIYEH LHSRNKKKNI |
| 541 | PTYLRQRIEK QLGEPSSRHV NVKGRYVRCQ DCPYKKDRKT KRSCNACAKP ICMEHAKFLC |
| 601 | ENCAELDSSL. |

In certain embodiments, the hyperactive piggyBac or piggyBac-like transposase is more active than the transposase of SEQ ID NO: 14505. In certain embodiments, the hyperactive piggyBac or piggyBac-like transposase is at least 90%, at least 95%, at least 96%, at least 97%, at least 98%, or at least 99% or any percentage in between identical to SEQ ID NO: 14505.

In certain embodiments, the hyperactive piggyBac or piggyBac-like transposase comprises an amino acid substitution at a position selected from 92, 93, 96, 97, 165, 178, 189, 196, 200, 201, 211, 215, 235, 238, 246, 253, 258, 261, 263, 271, 303, 321, 324, 330, 373, 389, 399, 402, 403, 404, 448, 473, 484, 507, 523, 527, 528, 543, 549, 550, 557, 601, 605, 607, 609, 610 or a combination thereof (relative to SEQ ID NO: 14505). In certain embodiments, the hyperactive piggyBac or piggyBac-like transposase comprises an amino acid substitution of Q92A, V93L, V93M, P96G, F97H, F97C, H165E, H165W, E178S, E178H, C189P, A196G, L200I, A201Q, L211A, W215Y, G2195, Q235Y, Q235G, Q238L, K246I, K253V, M258V, F261L, S263K, C271S, N303R, F321W, F321D, V324K, V324H, A330V, L373C, L373V, V389L, S399N, R402K, T403L, D404Q, D404S, D404M, N441R, G448W, E449A, V469T, C473Q, R484K T507C, G523A, I527M, Y528K Y543I, E549A, K550M, P557S, E601V, E605H, E605W, D607H, 5609H, L610I or any combination thereof. In certain embodiments, the hyperactive piggyBac or piggyBac-like transposase comprises an amino acid substitution of Q92A, V93L, V93M, P96G, F97H, F97C, H165E, H165W, E178S, E178H, C189P, A196G, L200I, A201Q, L211A, W215Y, G2195, Q235Y, Q235G, Q238L, K246I, K253V, M258V, F261L, S263K, C271S, N303R, F321W, F321D, V324K, V324H, A330V, L373C, L373V, V389L, S399N, R402K, T403L, D404Q, D404S, D404M, N441R, G448W, E449A, V469T, C473Q, R484K T507C, G523A, I527M, Y528K Y543I, E549A, K550M, P557S, E601V, E605H, E605W, D607H, 5609H and L610I.

In certain embodiments, the hyperactive piggyBac or piggyBac-like transposase comprises one or more substitutions of an amino acid that is not wild type, wherein the one or more substitutions a for wild type amino acid comprises a substitution of E4X, A12X, M13X, L14X, E15X, D20X, E24X, S25X, S26X, S27X, D32X, H33X, E36X, E44X, E45X, E46X, I48X, D49X, R58X, A62X, N63X, A64X, I65X, I66X, N68X, E69X, D71X, S72X, D76X, P79X, R84X, Q85X, A87X, S88X, Q92X, V93X, S94X, G95X, P96X, F97X, Y98X, T99X, I145X, S149X, D150X, L152X, E154X, T157X, N160X, S161X, S162X, H165X, R166X, T168X, K169X, T170X, A171X, E173X, S175X, S176X, E178X, T179X, M183X, Q184X, T186X, T187X, L188X, C189X, L194X, I195X, A196X, L198X, L200X, A201X, L203X, I204X, K205X, A206X, N207X, Q209X, S210X, L211X, K212X, D213X, L214X, W215X, R216X, T217X, G219X, V222X, D223X, I224X, T227X, M229X, Q235X, L237X, Q238X, N239X, N240X, P302X, N303X, P305X, A306X, K307X, Y308X, I310X, K311X, I312X, L313X, A314X, L315X, V316X, D317X, A318X, K319X, N320X, F321X, Y322X, V323X, V324X, L326X, E327X, V328X, A330X, Q333X, P334X, S335X, G336X, P337X, A339X, V340X, S341X, N342X, R343X, P344X, F345X, E346X, V347X, E349X, I352X, Q353X, V355X, A356X, R357X, N361X, D365X, W367X, T369X, G370X, L373X, M374X, L375X, H376X, N379X, E380X, R382X, V386X, V389X, N392X, R394X, Q395X, S399X, F400X, I401X, R402XT403X, D404X, R405X, Q406X, P407X, N408X, S409X, S410X, V411X, F412X, F414X, Q415X, I418X, T419X, L420X, N428XV432X, M434X, D440X, N441X, S442X, I443X, D444X, E445X, G448X, E449X, Q451X, K452X, M455X, I456X, T457X, F458X, S461X, A464X, V466X, Q468X, V469X, E471X, L472X, C473X, A474X, K483X, W485X, T488X, L489X, Y491X, G492X, V493X, M496X, I499X, C502X, I503X, T507X, K509X, N510X, V511X, T512X, I513X, R515X, E517X, S521X, G523X, L524X, S525X, I527X, Y528X, E529X, H532X, S533X, N535X, K536X, K537X, N539X, I540X, T542X, Y543X, Q546X, E549X, K550X, Q551X, G553X, E554X, P555X, S556X, P557X, R558X, H559X, V560X, N561X, V562X, P563X, G564X, R565X, Y566X, V567X, Q570X, D571X, P573X, Y574X, K576X, K581X, S583X, A586X, A588X, E594X, F598X, L599X, E601X, N602X, C603X, A604X, E605X, L606X, D607X, S608X, S609X or L610X (relative to SEQ ID NO: 14505). A list of hyperactive amino acid substitutions can be found in U.S. Pat. No. 10,041,077, the contents of which are incorporated herein by reference in their entirety.

In certain embodiments, the piggyBac or piggyBac-like transposase is integration deficient. In certain embodiments, an integration deficient piggyBac or piggyBac-like transposase is a transposase that can excise its corresponding transposon, but that integrates the excised transposon at a lower frequency than a corresponding wild type transposase. In certain embodiments, the piggyBac or piggyBac-like transposase is an integration deficient variant of SEQ ID NO: 14505.

In certain embodiments, the excision competent, integration deficient piggyBac or piggyBac-like transposase comprises one or more substitutions of an amino acid that is not wild type, wherein the one or more substitutions a for wild type amino acid comprises a substitution of R9X, A12X, M13X, D20X, Y21K, D23X, E24X, 525X, S26X, S27X, E28X, E30X, D32X, H33X, E36X, H37X, A39X, Y41X, D42X, T43X, E44X, E45X, E46X, R47X, D49X, S50X, 555X, A62X, N63X, A64X, I66X, A67X, N68X, E69X, D70X, D71X, S72X, D73X, P74X, D75X, D76X, D77X, I78X, 581X, V83X, R84X, Q85X, A87X, S88X, A89X, 590X, R91X, Q92X, V93X, S94X, G95X, P96X, F97X, Y98X, T99X, W012X, G103X, Y107X, K108X, L117X, I122X, Q128X, I312X, D135X, 5137X, E139X, Y140X, I145X, 5149X, D150X, Q153X, E154X, T157X, 5161X, 5162X, R164X, H165X, R166X, Q167X, T168X, K169X, T170X, A171X, A172X, E173X, R174X, 5175X, 5176X, A177X, E178X, T179X, 5180X, Y182X, Q184X, E185X, T187X, L188X, C189X, L194X, I195X, A196X, L198X, L200X, A201X, L203X, I204X, K205X, N207X, Q209X, L211X, D213X, L214X, W215X, R216X, T217X, G219X, T220X, V222X, D223X, I224X, T227X, T228X, F234X, Q235X, L237X, Q238X, N239X, N240X, N303X, K304X, I310X, I312X, L313X, A314X, L315X, V316X, D317X, A318X, K319X, N320X, F321X, Y322X, V323X, V324X, N325X, L326X, E327X, V328X, A330X, G331X, K332X, Q333X, 5335X, P337X, P344X, F345X, E349X, H359X, N361X, V362X, D365X, F368X, Y371X, E372X, L373X, H376X, E380X, R382X, R382X, V386X, G387X, T388X, V389X, K391X, N392X, R394X, Q395X, E398X, 5399X, F400X, I401X, R402XT403X, D404X, R405X, Q406X, P407X, N408X, 5409X, 5410X, Q415X, K416X, A424X, K426X, N428X, V430X, V432X, V433X, M434X, D436X, D440X, N441X, 5442X, I443X, D444X, E445X, 5446X, T447X, G448X, E449X, K450X, Q451X, E454X, M455X, I456X, T457X, F458X, 5461X, A464X, V466X, Q468X, V469X, C473X, A474X, N475X, N477X, K483X, R484X, P486X, T488X, L489X, G492X, V493X, M496X, I499X, I503X, Y505X, T507X, N510X, V511X, T512X, I513X, K514X, T516X, E517X, 5521X, G523X, L524X, 5525X, I527X, Y528X, L531X, H532X, S533X, N535X, I540X, T542X, Y543X, R545X, Q546X, E549X, L552X, G553X, E554X, P555X, S556X, P557X, R558X, H559X, V560X, N561X, V562X, P563X, G564X, V567X, Q570X, D571X, P573X, Y574X, K575X, K576X, N585X, A586X, M593X, K596X, E601X, N602X, A604X, E605X, L606X, D607X, S608X, S609X or L610X (relative to SEQ ID NO: 14505). A list of integration deficient amino acid substitutions can be found in U.S. Pat. No. 10,041,077, the contents of which are incorporated by reference in their entirety.

In certain embodiments, the integration deficient piggyBac or piggyBac-like transposase comprises a sequence of:

| (SEQ ID NO: 14606) | |
|---|---|
| 1 | MDIERQEERI RAMLEEELSD YSDESSSEDE TDHCSEHEVN YDTEEERIDS VDVPSNSRQE |
| 61 | EANAIIANES DSDPDDDLPL SLVRQRASAS RQVSGPFYTS KDGTKWYKNC QRPNVRLRSE |
| 121 | NIVTEQAQVK NIARDASTEY ECWNIFVTSD MLQEILTHTN SSIRHRQTKT AAENSSAETS |
| 181 | FYMQETTLCE LKALIALLYL AGLIKSNRQS LKDLWRKDGT GVDIFRTTMS LQRFQFLLNN |
| 241 | IRFDDISTRD ERKQTDNMAA FRSIFDQFVQ CCQNAYSPSE FLTIDEMLLS FRGRCLFRVY |
| 301 | IPNKPAKYGI KILALVDAKN FYVVNLEVYA GKQPSGPYAV SNRPFEVVER LIQPVARSHR |
| 361 | NVTFDNWFTG YELMLHLLNE YRLTSVGTVR KNKRQIPESF IRTDRQPNSS VFGFQKDITL |
| 421 | VSYAPKKNKV VVVMSTMHHD NSIDESTGEK QKPEMITFYN STKAGVDVVD ELCANYNVSR |
| 481 | NSKKWPMTLF YGVLNMAAIN ACIIYRTNKN VTIKRTEFIR SLGLSMMYEH LHSRNKKKNI |
| 541 | PTYLRQRIEK QLGEPVPRHV NVPGRYVRCQ DCPYKKDRKT KRSCNACAKP ICMEHAKFLC |
| 601 | ENCAELDSSL. |

In certain embodiments, the integration deficient piggyBac or piggyBac-like transposase comprises a sequence of:

| (SEQ ID NO: 14607) | |
|---|---|
| 1 | MDIERQEERI RAMLEEELSD YSDESSSEDE TDHCSEHEVN YDTEEERIDS VDVPSNSRQE |
| 61 | EANAIIANES DSDPDDDLPL SLVRQRASAS RQVSGPFYTS KDGTKWYKNC QRPNVRLRSE |
| 121 | NIVTEQAQVK NIARDASTEY ECWNIFVTSD MLQEILTHTN SSIRHRQTKT AAENSSAETS |
| 181 | FYMQETTLCE LKALIGLLYL AGLIKSNRQS LKDLWRTDGT GVDIFRTTMS LQRFYFLQNN |
| 241 | IRFDDKSTLD ERKQTDNMAA FRSIFDQFVQ SCQNAYSPSE FLTIDEMLLS FRGRCLFRVY |
| 301 | IPNKPAKYGI KILALVDAKN FYVVNLEVYA GKQPSGPYAV SNRPFEVVER LIQPVARSHR |
| 361 | NVTFDNWFTG YELMLHLLNE YRLTSVGTVR KNKRQIPESF IRTDRQPNSS VFGFQKDITL |
| 421 | VSYAPKKNKV VVVMSTMHHD NSIDESTGEK QKPEMITFYN STKAGVDVVD ELCANYNVSR |
| 481 | NSKRWPMTLF YGVLNMAAIN ACIIYRTNKN VTIKRTEFIR SLGLSMIYEH LHSRNKKKNI |
| 541 | PTYLRQRIEK QLGEPSPRHV NYPGRYVRCQ DCPYKKDRKT KRSCNACAKP ICMEHAKFLC |
| 601 | VNCAELDSSL. |

In certain embodiments, the piggyBac or piggyBac-like transposase that is is integration deficient comprises a sequence of:

| (SEQ ID NO: 14608) | |
|---|---|
| 1 | MDIERQEERI RAMLEEELSD YSDESSSEDE TDHCSEHEVN YDTEEERIDS VDVPSNSRQE |
| 61 | EANAIIANES DSDPDDDLPL SLVRQRASAS RQVSGPFYTS KDGTKWYKNC QRPNVRLRSE |
| 121 | NIVTEQAQVK NIARDASTEY ECWNIFVTSD MLQEILTHTN SSIRHRQTKT AAENSSAETS |
| 181 | FYMQETTLCE LKALIALLYL AGLIKSNRQS LKDLWRKDGT GVDIFRTTMS LQRFQFLLNN |
| 241 | IRFDDKSTRD ERKQTDNMAA FRSIFDQFVQ CCQNAYSPSE FLTIDEMLLS FRGRCLFRVY |
| 301 | IPNKPAKYGI KILALVDAKN DYVVNLEVYA GKQPSGPYAV SNRPFEVVER LIQPVARSHR |
| 361 | NVTFDNWFTG YECMLHLLNE YRLTSVGTVR KNKRQIPESF IRTDRQPNSS VFGFQKDITL |
| 421 | VSYAPKKNKV VVVMSTMHHD NSIDESTGEK QKPEMITFYN STKAGVDVVD ELCANYNVSR |
| 481 | NSKKWPMTLF YGVLNMAAIN ACIIYRTNKN VTIKRTEFIR SLGLSMIKEH LHSRNKKKNI |
| 541 | PTYLRQRIEK QLGEPSPRHV NVPGRYVRCQ DCPYKKDRKT KRSCNACAKP ICMEHAKFLC |
| 601 | ENCAELDSSL. |

In certain embodiments, the integration deficient transposase comprises a sequence that is at least 90% identical to SEQ ID NO: 14608.

In certain embodiments, the piggyBac or piggyBac-like transposon is isolated or derived from Bombyx mori. In certain embodiments, the piggyBac or piggyBac-like transposon comprises a sequence of:

| (SEQ ID NO: 14506) | |
|---|---|
| 1 | ttatcccggc gagcatgagg cagggtatct cataccctgg taaaatttta aagttgtgta |
| 61 | ttttataaaa ttttcgtctg acaacactag cgcgctcagt agctggaggc aggagcgtgc |
| 121 | gggaggggat agtggcgtga tcgcagtgtg gcacgggaca ccggcgagat attcgtgtgc |
| 181 | aaacctgttt cgggtatgtt ataccctgcc tcattgttga cgtatttttt ttatgtaatt |
| 241 | tttccgatta ttaatttcaa ctgttttatt ggtattttta tgttatccat tgttcttttt |
| 301 | ttatgattta ctgtatcggt tgtctttcgt tcctttagtt gagttttttt ttattatttt |
| 361 | cagtttttga tcaaa. |

In certain embodiments, the piggyBac or piggyBac-like transposon comprises a sequence of:

| (SEQ ID NO: 14507) | |
|---|---|
| 1 | tcatattttt agtttaaaaa aataattata tgttttataa tgaaaagaat ctcattatct |
| 61 | ttcagtatta ggttgattta tattccaaag aataatattt ttgttaaatt gttgattttt |
| 121 | gtaaacctct aaatgtttgt tgctaaaatt actgtgttta agaaaaagat taataaataa |
| 181 | taataatttc ataattaaaa acttctttca ttgaatgcca ttaaataaac cattatttta |
| 241 | caaaataaga tcaacataat tgagtaaata ataataagaa caatattata gtacaacaaa |
| 301 | atatgggtat gtcataccct gccacattct tgatgtaact ttttttcacc tcatgctcgc |
| 361 | cgggttat. |

In certain embodiments, the piggyBac or piggyBac-like transposon comprises a sequence of:

| (SEQ ID NO: 14508) | |
|---|---|
| 1 | ttatcccggc gagcatgagg cagggtatct cataccctgg taaaatttta aagttgtgta |
| 61 | ttttataaaa ttttcgtctg acaacactag cgcgctcagt agctggaggc aggagcgtgc |
| 121 | gggaggggat agtggcgtga tcgcagtgtg gcacgggaca ccggcgagat attcgtgtgc |
| 181 | aaacctgttt cgggtatgtt ataccctgcc tcat. |

In certain embodiments, the piggyBac™ (PB) or piggyBac-like transposon comprises a sequence of:

| (SEQ ID NO: 14509) | |
|---|---|
| 1 | taaataataa taatttcata attaaaaact tctttcattg aatgccatta aataaaccat |
| 61 | tattttacaa aataagatca acataattga gtaaataata ataagaacaa tattatagta |
| 121 | caacaaaata tgggtatgtc ataccctgcc acattcttga tgtaactttt tttcacctca |
| 181 | tgctcgccgg gttat. |

In certain embodiments, the piggyBac or piggyBac-like transposon comprises a left sequence corresponding to SEQ ID NO: 14506 and a right sequence corresponding to SEQ ID NO: 14507. In certain embodiments, one piggyBac or piggyBac-like transposon end is at least 85%, at least 90%, at least 95%, at least 98%, at least 99% identical or any percentage in between identical to SEQ ID NO: 14506 and the other piggyBac or piggyBac-like transposon end is at least 85%, at least 90%, at least 95%, at least 98%, at least 99% or any percentage in between identical to SEQ ID NO: 14507. In certain embodiments, the piggyBac or piggyBac-like transposon comprises SEQ ID NO: 14506 and SEQ ID NO: 14507 or SEQ ID NO: 14509. In certain embodiments, the piggyBac or piggyBac-like transposon comprises SEQ ID NO: 14508 and SEQ ID NO: 14507 or SEQ ID NO: 14509. In certain embodiments, the left and right transposon ends share a 16 bp repeat sequence at their ends of CCCGGCGAGCATGAGG (SEQ ID NO: 14510) immediately adjacent to the 5′-TTAT-3 target insertion site, which is inverted in the orientation in the two ends. In certain embodiments, left transposon end begins with a sequence comprising 5′-TTATCCCGGCGAGCATGAGG-3 (SEQ ID NO: 14511), and the right transposon ends with a sequence comprising the reverse complement of this sequence:

(SEQ ID NO: 14512)
5′-CCTCATGCTCGCCGGGTTAT-3′.

In certain embodiments, the piggyBac or piggyBac-like transposon comprises one end comprising at least 14, 16, 18, 20, 30 or 40 contiguous nucleotides of SEQ ID NO: 14506 or SEQ ID NO: 14508. In certain embodiments, the piggyBac or piggyBac-like transposon comprises one end comprising at least 14, 16, 18, 20, 30 or 40 contiguous nucleotides of SEQ ID NO: 14507 or SEQ ID NO: 14509. In certain embodiments, the piggyBac or piggyBac-like transposon comprises one end with at least 90% identity to SEQ ID NO: 14506 or SEQ ID NO: 14508. In certain embodiments, the piggyBac or piggyBac-like transposon comprises one end with at least 90% identity to SEQ ID NO: 14507 or SEQ ID NO: 14509.

In certain embodiments, the piggyBac or piggyBac-like transposon comprises a sequence of:

| (SEQ ID NO: 14515) | |
|---|---|
| 1 | ttaacccggc gagcatgagg cagggtatct cataccctgg taaaatttta aagttgtgta |
| 61 | ttttataaaa ttttcgtctg acaacactag cgcgctcagt agctggaggc aggagcgtgc |
| 121 | gggaggggat agtggcgtga tcgcagtgtg gcacgggaca ccggcgagat attcgtgtgc |
| 181 | aaacctgttt cgggtatgtt ataccctgcc tcattgttga cgtatttttt ttatgtaatt |
| 241 | tttccgatta ttaatttcaa ctgttttatt ggtattttta tgttatccat tgttcttttt |
| 301 | ttatgattta ctgtatcggt tgtctttcgt tcctttagtt gagttttttt ttattatttt |
| 361 | cagtttttga tcaaa. |

In certain embodiments, the piggyBac or piggyBac-like transposon comprises a sequence of:

| (SEQ ID NO: 14516) | |
|---|---|
| 1 | tcatattttt agtttaaaaa aataattata tgttttataa tgaaaagaat ctcattatct |
| 61 | ttcagtatta ggttgattta tattccaaag aataatattt ttgttaaatt gttgattttt |
| 121 | gtaaacctct aaatgtttgt tgctaaaatt actgtgttta agaaaaagat taataaataa |
| 181 | taataatttc ataattaaaa acttctttca ttgaatgcca ttaaataatt cattatttta |
| 241 | caaaataaga tcaacataat tgagtaaata ataataagaa caatattata gtacaacaaa |
| 301 | atatgggtat gtcataccct tttttttttt tttttttttt ttttttcggg tagagggccg |
| 361 | aacctcctac gaggtccccg cgcaaaaggg gcgcgcgggg tatgtgagac tcaacgatct |
| 421 | gcatggtgtt gtgagcagac cgcgggccca aggattttag agcccaccca ctaaacgact |
| 481 | cctctgcact cttacacccg acgtccgatc ccctccgagg tcagaacccg gatgaggtag |
| 541 | gggggctacc gcggtcaaca ctacaaccag acggcgcggc tcaccccaag gacgcccagc |
| 601 | cgacggagcc ttcgaggcga atcgaaggct ctgaaacgtc ggccgtctcg gtacggcagc |
| 661 | ccgtcgggcc gcccagacgg tgccgctggt gtcccggaat accccgctgg accagaacca |
| 721 | gcctgccggg tcgggacgcg atacaccgtc gaccggtcgc tctaatcact ccacggcagc |
| 781 | gcgctagagt gctggta. |

In certain embodiments, the piggyBac or piggyBac-like transposon comprises a sequence of CCCGGCGAGCATGAGG (SEQ ID NO: 14510). In certain embodiments, the piggyBac or piggyBac-like transposon comprises an ITR sequence of SEQ ID NO: 14510. In certain embodiments, the piggyBac or piggyBac-like transposon comprises a sequence of TTATCCCGGCGAGCATGAGG (SEQ ID NO: 14511). In certain embodiments, the piggyBac or piggyBac-like transposon comprises at least 16 contiguous nucleotides from SEQ ID NO: 14511. In certain embodiments, the piggyBac or piggyBac-like transposon comprises a sequence of CCTCATGCTCGCCGGGTTAT (SEQ ID NO: 14512). In certain embodiments, the piggyBac or piggyBac-like transposon comprises at least 16 contiguous nucleotides from SEQ ID NO: 14512. In certain embodiments, the piggyBac or piggyBac-like transposon comprises one end comprising at least 16 contiguous nucleotides from SEQ ID NO: 14511 and one end comprising at least 16 contiguous nucleotides from SEQ ID NO: 14512. In certain embodiments, the piggyBac or piggyBac-like transposon comprises SEQ ID NO: 14511 and SEQ ID NO: 14512. In certain embodiments, the piggyBac or piggyBac-like transposon comprises a sequence of TTAACCCGGCGAGCATGAGG (SEQ ID NO: 14513). In certain embodiments, the piggyBac or piggyBac-like transposon comprises a sequence of CCTCATGCTCGCCGGGTTAA (SEQ ID NO: 14514).

In certain embodiments, the piggyBac or piggyBac-like transposon may have ends comprising SEQ ID NO: 14506 and SEQ ID NO: 14507, or a variant of either or both of these having at least 90% sequence identity to SEQ ID NO: 14506 or SEQ ID NO: 14507, and the piggyBac or piggyBac-like transposase has the sequence of SEQ ID NO: 14504 or SEQ ID NO: 14505, or a sequence at least 5%, 10%, 15%, 20%, 25%, 30%, 35%, 40%, 45%, 50%, 55%, 60%, 65%, 70%, 75%, 80%, 85%, 90%, 95%, 99% or any percentage in between identity to SEQ ID NO: 14504 or SEQ ID NO: 14505. In certain embodiments, the piggyBac or piggyBac-like transposon comprises a heterologous polynucleotide inserted between a pair of inverted repeats, where the transposon is capable of transposition by a piggyBac or piggyBac-like transposase having at least 5%, 10%, 15%, 20%, 25%, 30%, 35%, 40%, 45%, 50%, 55%, 60%, 65%, 70%, 75%, 80%, 85%, 90%, 95%, 99% or any percentage in between identity to SEQ ID NO: 14504 or SEQ ID NO: 14505. In certain embodiments, the transposon comprises two transposon ends, each of which comprises SEQ ID NO: 14510 in inverted orientations in the two transposon ends. In certain embodiments, each inverted terminal repeat (ITR) is at least 90% identical to SEQ ID NO: 14510.

In certain embodiments, the piggyBac or piggyBac-like transposon is capable of insertion by a piggyBac or piggyBac-like transposase at the sequence 5′-TTAT-3 within a target nucleic acid. In certain embodiments, one end of the piggyBac or piggyBac-like transposon comprises at least 16 contiguous nucleotides from SEQ ID NO: 14506 and the other transposon end comprises at least 16 contiguous nucleotides from SEQ ID NO: 14507. In certain embodiments, one end of the piggyBac or piggyBac-like transposon comprises at least 17, at least 18, at least 19, at least 20, at least 22, at least 25, at least 30 contiguous nucleotides from SEQ ID NO: 14506 and the other transposon end comprises at least 17, at least 18, at least 19, at least 20, at least 22, at least 25, at least 30 contiguous nucleotides from SEQ ID NO: 14507.

In certain embodiments, the piggyBac or piggyBac-like transposon comprises transposon ends (each end comprising an ITR) corresponding to SEQ ID NO: 14506 and SEQ ID NO: 14507, and has a target sequence corresponding to 5′-TTAT3′. In certain embodiments, the piggyBac or piggyBac-like transposon also comprises a sequence encoding a transposase (e.g. SEQ ID NO: 14505). In certain embodiments, the piggyBac or piggyBac-like transposon comprises one transposon end corresponding to SEQ ID NO: 14506 and a second transposon end corresponding to SEQ ID NO: 14516. SEQ ID NO: 14516 is very similar to SEQ ID NO: 14507, but has a large insertion shortly before the ITR. Although the ITR sequences for the two transposon ends are identical (they are both identical to SEQ ID NO: 14510), they have different target sequences: the second transposon has a target sequence corresponding to 5′-TTAA-3′, providing evidence that no change in ITR sequence is necessary to modify the target sequence specificity. The piggyBac or piggyBac-like transposase (SEQ ID NO: 14504), which is associated with the 5′-TTAA-3′ target site differs from the 5′-TTAT-3′-associated transposase (SEQ ID NO: 14505) by only 4 amino acid changes (D322Y, S473C, A507T, H582R). In certain embodiments, the piggyBac or piggyBac-like transposase (SEQ ID NO: 14504), which is associated with the 5′-TTAA-3′ target site is less active than the 5′-TTAT-3′-associated piggyBac or piggyBac-like transposase (SEQ ID NO: 14505) on the transposon with 5′-TTAT-3′ ends. In certain embodiments, piggyBac or piggyBac-like transposons with 5′-TTAA-3′ target sites can be converted to piggyBac or piggyBac-like transposases with 5′-TTAT-3 target sites by replacing 5′-TTAA-3′ target sites with 5′-TTAT-3′. Such transposons can be used either with a piggyBac or piggyBac-like transposase such as SEQ ID NO: 14504 which recognizes the 5′-TTAT-3′ target sequence, or with a variant of a transposase originally associated with the 5′-TTAA-3′ transposon. In certain embodiments, the high similarity between the 5′-TTAA-3′ and 5′-TTAT-3′ piggyBac or piggyBac-like transposases demonstrates that very few changes to the amino acid sequence of a piggyBac or piggyBac-like transposase alter target sequence specificity. In certain embodiments, modification of any piggyBac or piggyBac-like transposon-transposase gene transfer system, in which 5′-TTAA-3′ target sequences are replaced with 5′-TTAT-3′-target sequences, the ITRs remain the same, and the transposase is the original piggyBac or piggyBac-like transposase or a variant thereof resulting from using a low-level mutagenesis to introduce mutations into the transposase. In certain embodiments, piggyBac or piggyBac-like transposon transposase transfer systems can be formed by the modification of a 5′-TTAT-3′-active piggyBac or piggyBac-like transposon-transposase gene transfer systems in which 5′-TTAT-3′ target sequences are replaced with 5′-TTAA-3′-target sequences, the ITRs remain the same, and the piggyBac or piggyBac-like transposase is the original transposase or a variant thereof.

In certain embodiments, the piggyBac or piggyBac-like transposon is isolated or derived from Bombyx mori. In certain embodiments, the piggyBac or piggyBac-like transposon comprises a sequence of:

| (SEQ ID NO: 14577) | |
|---|---|
| 1 | cccggcgagc atgaggcagg gtatctcata ccctggtaaa attttaaagt tgtgtatttt |
| 61 | ataaaatttt cgtctgacaa cactagcgcg ctcagtagct ggaggcagga gcgtgcggga |
| 121 | ggggatagtg gcgtgatcgc agtgtggcac gggacaccgg cgagatattc gtgtgcaaac |
| 181 | ctgtttcggg tatgttatac cctgcctcat tgttgacgta t. |

In certain embodiments, the piggyBac or piggyBac-like transposon comprises a sequence of:

| (SEQ ID NO: 14578) | |
|---|---|
| 1 | tttaagaaaa agattaataa ataataataa tttcataatt aaaaacttct ttcattgaat |
| 61 | gccattaaat aaaccattat tttacaaaat aagatcaaca taattgagta aataataata |
| 121 | agaacaatat tatagtacaa caaaatatgg gtatgtcata ccctgccaca ttcttgatgt |
| 181 | aacttttttt cacctcatgc tcgccggg. |

In certain embodiments, the transposon comprises at least 16 contiguous bases from SEQ ID NO: 14577 and at least 16 contiguous bases from SEQ ID NO: 14578, and inverted terminal repeats that are at least 87% identical to CCCGGCGAGCATGAGG (SEQ ID NO: 14510). In certain embodiments, the piggyBac or piggyBac-like transposon comprises a sequence of:

| (SEQ ID NO: 14595) | |
|---|---|
| 1 | cccggcgagc atgaggcagg gtatctcata ccctggtaaa attttaaagt tgtgtatttt |
| 61 | ataaaatttt cgtctgacaa cactagcgcg ctcagtagct ggaggcagga gcgtgcggga |
| 121 | ggggatagtg gcgtgatcgc agtgtggcac gggacaccgg cgagatattc gtgtgcaaac |
| 181 | ctgtttcggg tatgttatac cctgcctcat tgttgacgta ttttttttat gtaatttttc |
| 241 | cgattattaa tttcaactgt tttattggta tttttatgtt atccattgtt ctttttttat |
| 301 | gatttactgt atcggttgtc tttcgttcct ttagttgagt ttttttttat tattttcagt |
| 361 | ttttgatcaa a. |

In certain embodiments, the piggyBac or piggyBac-like transposon comprises a sequence of:

| (SEQ ID NO: 14596) | |
|---|---|
| 1 | tcatattttt agtttaaaaa aataattata tgttttataa tgaaaagaat ctcattatct |
| 61 | ttcagtatta ggttgattta tattccaaag aataatattt ttgttaaatt gttgattttt |
| 121 | gtaaacctct aaatgtttgt tgctaaaatt actgtgttta agaaaaagat taataaataa |
| 181 | taataatttc ataattaaaa acttctttca ttgaatgcca ttaaataaac cattatttta |
| 241 | caaaataaga tcaacataat tgagtaaata ataataagaa caatattata gtacaacaaa |
| 301 | atatgggtat gtcataccct gccacattct tgatgtaact ttttttcacc tcatgctcgc |
| 361 | cggg. |

In certain embodiments, the piggyBac or piggyBac-like transposon comprises SEQ ID NO: 14595 and SEQ ID NO: 14596, and is transposed by the piggyBac or piggyBac-like transposase of SEQ ID NO: 14505. In certain embodiments, the ITRs of SEQ ID NO: 14595 and SEQ ID: 14596 are not flanked by a 5′-TTAA-3′ sequence. In certain embodiments, the ITRs of SEQ ID NO: 14595 and SEQ ID: 14596 are flanked by a 5′-TTAT-3′ sequence.

In certain embodiments, the piggyBac or piggyBac-like transposon comprises a sequence of:

| (SEQ ID NO: 14597) | |
|---|---|
| 1 | cccggcgagc atgaggcagg gtatctcata ccctggtaaa attttaaagt tgtgtatttt |
| 61 | ataaaatttt cgtctgacaa cactagcgcg ctcagtagct ggaggcagga gcgtgcggga |
| 121 | ggggatagtg gcgtgatcgc agtgtggcac gggacaccgg cgagatattc gtgtgcaaac |
| 181 | ctgtttcggg tatgttatac cctgcctcat tgttgacgta ttttttttat gtaatttttc |
| 241 | cgattattaa tttcaactgt tttattggta tttttatgtt atccattgtt ctttttttat |
| 301 | g. |

In certain embodiments, the piggyBac or piggyBac-like transposon comprises a sequence of:

| (SEQ ID NO: 14598) | |
|---|---|
| 1 | cagggtatct cataccctgg taaaatttta aagttgtgta ttttataaaa ttttcgtctg |
| 61 | acaacactag cgcgctcagt agctggaggc aggagcgtgc gggaggggat agtggcgtga |
| 121 | tcgcagtgtg gcacgggaca ccggcgagat attcgtgtgc aaacctgttt cgggtatgtt |
| 181 | ataccctgcc tcattgttga cgtatttttt ttatgtaatt tttccgatta ttaatttcaa |
| 241 | ctgttttatt ggtattttta tgttatccat tgttcttttt ttatg. |

In certain embodiments, the piggyBac or piggyBac-like transposon comprises a sequence of:

| (SEQ ID NO: 14599) | |
|---|---|
| 1 | cagggtatct cataccctgg taaaatttta aagttgtgta ttttataaaa ttttcgtctg |
| 61 | acaacactag cgcgctcagt agctggaggc aggagcgtgc gggaggggat agtggcgtga |
| 121 | tcgcagtgtg gcacgggaca ccggcgagat attcgtgtgc aaacctgttt cgggtatgtt |
| 181 | ataccctgcc tcattgttga cgtat. |

In certain embodiments, the left end of the piggyBac or piggyBac-like transposon comprises a sequence of SEQ ID NO: 14577, SEQ ID NO: 14595, or SEQ ID NOs: 14597-14599. In certain embodiments, the left end of the piggyBac or piggyBac-like transposon is preceded by a left target sequence.
In certain embodiments, the piggyBac or piggyBac-like transposon comprises a sequence of:

| (SEQ ID NO: 14600) | |
|---|---|
| 1 | tcatattttt agtttaaaaa aataattata tgttttataa tgaaaagaat ctcattatct |
| 61 | ttcagtatta ggttgattta tattccaaag aataatattt ttgttaaatt gttgattttt |
| 121 | gtaaacctct aaatgtttgt tgctaaaatt actgtgttta agaaaaagat taataaataa |
| 181 | taataatttc ataattaaaa acttctttca ttgaatgcca ttaaataaac cattatttta |
| 241 | caaaataaga tcaacataat tgagtaaata ataataagaa caatattata gtacaacaaa |
| 301 | atatgggtat gtcataccct gccacattct tgatgtaact ttttttcacc tcatgctcgc |
| 361 | cggg. |

In certain embodiments, the piggyBac or piggyBac-like transposon comprises a sequence of:

| (SEQ ID NO: 14601) | |
|---|---|
| 1 | tttaagaaaa agattaataa ataataataa tttcataatt aaaaacttct ttcattgaat |
| 61 | gccattaaat aaaccattat tttacaaaat aagatcaaca taattgagta aataataata |
| 121 | agaacaatat tatagtacaa caaaatatgg gtatgtcata ccctgccaca ttcttgatgt |
| 181 | aacttttttt ca. |

In certain embodiments, the piggyBac or piggyBac-like transposon comprises a sequence of:

| (SEQ ID NO: 14602) | |
|---|---|
| 1 | cccggcgagc atgaggcagg gtatctcata ccctggtaaa attttaaagt tgtgtatttt |
| 61 | ataaaatttt cgtctgacaa cactagcgcg ctcagtagct ggaggcagga gcgtgcggga |
| 121 | ggggatagtg gcgtgatcgc agtgtggcac gggacaccgg cgagatattc gtgtgcaaac |
| 181 | ctgtttcggg tatgttatac cctgcctcat tgttgacgta ttttttttat gtaatttttc |
| 241 | cgattattaa tttcaactgt tttattggta tttttatgtt atccattgtt ctttttttat |
| 301 | gatttactgt atcggttgtc tttcgttcct ttagttgagt ttttttttat tattttcagt |
| 361 | ttttgatcaa a. |

In certain embodiments, the right end of the piggyBac or piggyBac-like transposon comprises a sequence of SEQ ID NO: 14578, SEQ ID NO: 14596, or SEQ ID NOs: 14600-14601. In certain embodiments, the right end of the piggyBac or piggyBac-like transposon is followed by a right target sequence. In certain embodiments, the transposon is transposed by the transposase of SEQ ID NO: 14505. In certain embodiments, the left and right ends of the piggyBac or piggyBac-like transposon share a 16 bp repeat sequence of SEQ ID NO: 14510 in inverted orientation and immediately adjacent to the target sequence. In certain embodiments, the left transposon end begins with SEQ ID NO: 14510, and the right transposon end ends with the reverse complement of SEQ ID NO: 14510, 5′-CCTCATGCTCGCCGGG-3′ (SEQ ID NO: 14603). In certain embodiments, the piggyBac or piggyBac-like transposon comprises an ITR with at least 93%, at least 87%, or at least 81% or any percentage in between identity to SEQ ID NO: 14510 or SEQ ID NO: 14603. In certain embodiments, the piggyBac or piggyBac-like transposon comprises a target sequence followed by a left transposon end comprising a sequence selected from SEQ ID NOs: 88, 105 or 107 and a right transposon end comprising SEQ ID NO: 14578 or 106 followed by a target sequence. in certain embodiments, the piggyBac or piggyBac like transposon comprises one end that comprises a sequence that is at least 90%, at least 95% or at least 99% or any percentage in between identical to SEQ ID NO: 14577 and one end that comprises a sequence that is at least 90%, at least 95% or at least 99% or any percentage in between identical to SEQ ID NO: 14578. In certain embodiments, one transposon end comprises at least 14, at least 16, at least 18 or at least 20 contiguous bases from SEQ ID NO: 14577 and one transposon end comprises at least 14, at least 16, at least 18 or at least 20 contiguous bases from SEQ ID NO: 14578.

In certain embodiments, the piggyBac or piggyBac-like transposon comprises two transposon ends wherein each transposon ends comprises a sequence that is at least 81% identical, at least 87% identical or at least 93% identical or any percentage in between identical to SEQ ID NO: 14510 in inverted orientation in the two transposon ends. One end may further comprise at least 14, at least 16, at least 18 or at least 20 contiguous bases from SEQ ID NO: 14599, and the other end may further comprise at least 14, at least 16, at least 18 or at least 20 contiguous bases from SEQ ID NO: 14601. The piggyBac or piggyBac-like transposon may be transposed by the transposase of SEQ ID NO: 14505, and the transposase may optionally be fused to a nuclear localization signal.

In certain embodiments, the piggyBac or piggyBac-like transposon comprises SEQ ID NO: 14595 and SEQ ID NO: 14596 and the piggyBac or piggyBac-like transposase comprises SEQ ID NO: 14504 or SEQ ID NO: 14505. In certain embodiments, the piggyBac or piggyBac-like transposon comprises SEQ ID NO: 14597 and SEQ ID NO: 14596 and the piggyBac or piggyBac-like transposase comprises SEQ ID NO: 14504 or SEQ ID NO: 14505. In certain embodiments, the piggyBac or piggyBac-like transposon comprises SEQ ID NO: 14595 and SEQ ID NO: 14578 and the piggyBac or piggyBac-like transposase comprises SEQ ID NO: 14504 or SEQ ID NO: 14505. In certain embodiments, the piggyBac or piggyBac-like transposon comprises SEQ ID NO: 14602 and SEQ ID NO: 14600 and the piggyBac or piggyBac-like transposase comprises SEQ ID NO: 14504 or SEQ ID NO: 14505.

In certain embodiments, the piggyBac or piggyBac-like transposon comprises a left end comprising 1, 2, 3, 4, 5, 6, or 7 sequences selected from ATGAGGCAGGGTAT (SEQ ID NO: 14614), ATACCCTGCCTCAT (SEQ ID NO: 14615), GGCAGGGTAT (SEQ ID NO: 14616), ATACCCTGCC (SEQ ID NO: 14617), TAAAATTTTA (SEQ ID NO: 14618), ATTTTATAAAAT (SEQ ID NO: 14619), TCATACCCTG (SEQ ID NO: 14620) and TAAATAATAATAA (SEQ ID NO: 14621). In certain embodiments, the piggyBac or piggyBac-like transposon comprises a right end comprising 1, 2 or 3 sequences selected from SEQ ID NO: 14617, SEQ ID NO: 14620 and SEQ ID NO: 14621.

In certain embodiments of the methods of the disclosure, the transposase enzyme is a piggyBac or piggyBac-like transposase enzyme. In certain embodiments, the piggyBac or piggyBac-like transposase enzyme is isolated or derived from Xenopus tropicalis. The piggyBac or piggyBac-like transposase enzyme may comprise or consist of an amino acid sequence at least 5%, 10%, 15%, 20%, 25%, 30%, 35%, 40%, 45%, 50%, 55%, 60%, 65%, 70%, 75%, 80%, 85%, 90%, 95%, 99% or any percentage in between identical to:

| (SEQ ID NO: 14517) | |
|---|---|
| 1 | MAKRFYSAEE AAAHCMASSS EEFSGSDSEY VPPASESDSS TEESWCSSST VSALEEPMEV |
| 61 | DEDVDDLEDQ EAGDRADAAA GGEPAWGPPC NFPPEIPPFT TVPGVKVDTS NFEPINFFQL |
| 121 | FMTEAILQDM VLYTNVYAEQ YLTQNPLPRY ARAHAWHPTD IAEMKRFVGL TLAMGLIKAN |
| 181 | SLESYWDTTT VLSIPVFSAT MSRNRYQLLL RFLHFNNNAT AVPPDQPGHD RLHKLRPLID |
| 241 | SLSERFAAVY TPCQNICIDE SLLLFKGRLQ FRQYIPSKRA RYGIKFYKLC ESSSGYTSYF |
| 301 | LIYEGKDSKL DPPGCPPDLT VSGKIVWELI SPLLGQGFHL YVDNFYSSIP LFTALYCLDT |
| 361 | PACGTINRNR KGLPRALLDK KLNRGETYAL RKNELLAIKF FDKKNVFMLT SIHDESVIRE |
| 421 | QRVGRPPKNK PLCSKEYSKY MGGVDRTDQL QHYYNATRKT RAWYKKVGIY LIQMALRNSY |
| 481 | IVYKAAVPGP KLSYYKYQLQ ILPALLFGGV EEQTVPEMPP SDNVARLIGK HFIDTLPPTP |
| 541 | GKQRPQKGCK VCRKRGIRRD TRYYCPKCPR NPGLCFKPCF EIYHTQLHY. |

In some embodiments, the piggyBac or piggyBac-like transposase is a hyperactive variant of SEQ ID NO: 14517. In certain embodiments, the piggyBac or piggyBac-like transposase is an integration defective variant of SEQ ID NO: 14517. The piggyBac or piggyBac-like transposase enzyme may comprise or consist of an amino acid sequence at least 5%, 10%, 15%, 20%, 25%, 30%, 35%, 40%, 45%, 50%, 55%, 60%, 65%, 70%, 75%, 80%, 85%, 90%, 95%, 99% or any percentage in between identical to:

| (SEQ ID NO: 14518) | |
|---|---|
| 1 | MAKRFYSAEE AAAHCMAPSS EEFSGSDSEY VPPASESDSS TEESWCSSST VSALEEPMEV |
| 61 | DEDVDDLEDQ EAGDRADAAA GGEPAWGPPC NFPPEIPPFT TVPGVKVDTS NFEPINFFQL |
| 121 | FMTEAILQDM VLYTNVYAEQ YLTQNPLPRY ARAHAWHPTD IAEMKRFVGL TLAMGLIKAN |
| 181 | SLESYWNTTT VLSIPVFSAT MSRNRYQLLL RFLHFNNNAT AVPPDQPDHD RLHKLRPLID |
| 241 | SLSERFAAVY TPCQNICIDE SLLLFKGRLR FRQYIPSKRA RYGIKFYKLC ESSSGYTSYF |
| 301 | LIYEGKDSKL DPPGCPPDLT VSGKIVWELI SPLLGQGFHL YVDNFYSSIP LFTALYCLDT |
| 361 | PACGTINRTR KGLPRALLDK KLNRGETYAL RKNELLAIKF FDKKNVFMLT SIHDESVIRE |
| 421 | QRVGRPPKNK PLCSKEYSKY MGGVDRTDQL QHYYNATRKT SAWYKKVGIY LIQMALRNSY |
| 481 | IVYKAAVPGP KLSYYKYQLQ ILPALLFGGV EEQTVPEMLP SDNVARLIGK HFIDTLPPTP |
| 541 | GKQRPQKGCK VCRKRGIRRD TRYYCPKCPR NPGLCFKPCF EIYHTQLHY. |

In certain embodiments, the piggyBac or piggyBac-like transposase is isolated or derived from Xenopus tropicalis. In certain embodiments, the piggyBac or piggyBac-like transposase is a hyperactive piggyBac or piggyBac-like transposase. In certain embodiments, the hyperactive piggyBac or piggyBac-like transposase comprises a sequence at least 90% identical to:

| (SEQ ID NO: 14572) | |
|---|---|
| 1 | MAKRFYSAEE AAAHCSASSS EEFSGSDSEY VPPASESDSS TEESWCSSST VSALEEPMEV |
| 61 | DEDVDDLEDQ EAGDRADAAA GGEPAWGPPC NFPPEIPPFT TVPGVKVDTS NFEPINFFQL |
| 121 | FMTEAILQDM VLYTNVYAEQ YLTQNPLTRG ARAHAWHPTD IAEMKRFVGL TLAMGLIKAN |
| 181 | SIESYWDTTT VLSIPVFGAT MSRNRYQLLL RFLHFNNNAT AVPPDQPGHD RLHKLRPLID |
| 241 | SLSERFANVY TPCQNICIDE SLMLFKGRLQ FRQYIPSKRA RYGIKFYKLC ESSTGYTSYF |
| 301 | LIYEGKDSKL DPPGCPPDLT VSGKIVWELI SPLLGQGFHL YVDNFYSSIP LFTALYCLNT |
| 361 | PACGTINRNR KGLPRALLDK KLNRGETYAL RKNELLAIKF FDKKNVFMLT SIHDESVIRE |
| 421 | QRVGRPPKNK PLCSKEYSKY MGGVDRTDQL QHYYNATRKT RHWYKKVGIY LIQMALRNSY |
| 481 | IVYKAAVPGP KLSYYKYQLQ ILPALLFGGV EEQTVPEMPD SDNVARLIGK HFIDTLPPTP |
| 541 | GKQRPQKGCK VCRKRGIRRD TRYYCPKCPR NPGLCRKPCF EIYHTQLHY. |

In certain embodiments, piggyBac or piggyBac-like transposase is a hyperactive piggyBac or piggyBac-like transposase. A hyperactive piggyBac or piggyBac-like transposase is a transposase that is more active than the naturally occurring variant from which it is derived. In certain embodiments, a hyperactive piggyBac or piggyBac-like transposase is more active than the transposase of SEQ ID NO: 14517. In certain embodiments, the hyperactive piggyBac or piggyBac-like transposase comprises a sequence of:

| (SEQ ID NO: 14572) | |
|---|---|
| 1 | MAKRFYSAEE AAAHCSASSS EEFSGSDSEY VPPASESDSS TEESWCSSST VSALEEPMEV |
| 61 | DEDVDDLEDQ EAGDRADAAA GGEPAWGPPC NFPPEIPPFT TVPGVKVDTS NFEPINFFQL |
| 121 | FMTEAILQDM VLYTNVYAEQ YLTQNPLTRG ARAHAWHPTD IAEMKRFVGL TLAMGLIKAN |
| 181 | SIESYWDTTT VLSIPVFGAT MSRNRYQLLL RFLHFNNNAT AVPPDQPGHD RLHKLRPLID |
| 241 | SLSERFANVY TPCQNICIDE SLMLFKGRLQ FRQYIPSKRA RYGIKFYKLC ESSTGYTSYF |
| 301 | LIYEGKDSKL DPPGCPPDLT VSGKIVWELI SPLLGQGFHL YVDNFYSSIP LFTALYCLNT |
| 361 | PACGTINRNR KGLPRALLDK KLNRGETYAL RKNELLAIKF FDKKNVFMLT SIHDESVIRE |
| 421 | QRVGRPPKNK PLCSKEYSKY MGGVDRTDQL QHYYNATRKT RHWYKKVGIY LIQMALRNSY |
| 481 | IVYKAAVPGP KLSYYKYQLQ ILPALLFGGV EEQTVPEMPD SDNVARLIGK HFIDTLPPTP |
| 541 | GKQRPQKGCK VCRKRGIRRD TRYYCPKCPR NPGLCRKPCF EIYHTQLHY. |

In certain embodiments, the hyperactive piggyBac or piggyBac-like transposase comprises a sequence of:

| (SEQ ID NO: 14624) | |
|---|---|
| 1 | MAKRFYSAEE AAAHCMASSS EEFSGSDSEY VPPASESDSS TEESWCSSST VSALEEPMEV |
| 61 | DEDVDDLEDQ EAGDRADAAA GGEPAWGPPC NFPPEIPPFT TVPGVKVDTS NFEPINFFQL |
| 121 | FMTEAILQDM VLYTNVYAEQ YLTQNPLTRY ARAHAWHPTD IAEMKRFVGL TLAMGLIKAN |
| 181 | SLESYWDTTT VLSIPVFSAT MSRNRYQLLL RFLHFNNNAT AVPPDQPGHD RLHKLRPLID |
| 241 | SLSERFAAVY TPCQNICIDE SLLLFKGRLQ FRQYIPSKRA RYGIKFYKLC ESSSGYTSYF |
| 301 | LIYEGKDSKL DPPGCPPDLT VSGKIVWELI SPLLGQGFHL YVDNFYSSIP LFTALYCLNT |
| 361 | PACGTINRNR KGLPRALLDK KLNRGETYAL RKNELLAIKF FDKKNVFMLT SIHDESVIRE |
| 421 | QRVGRPPKNK PLCSKEYSKY MGGVDRTDQL QHYYNATRKT RHWYKKVGIY LIQMALRNSY |
| 481 | IVYKAAVPGP KLSYYKYQLQ ILPALLFGGV EEQTVPEMPP SDNVARLIGK HFIDTLPPTP |
| 541 | GKQRPQKGCK VCRKRGIRRD TRYYCPKCPR NPGLCRKPCF EIYHTQLHY. |

In certain embodiments, the hyperactive piggyBac or piggyBac-like transposase comprises a sequence of:

| (SEQ ID NO: 14625) | |
|---|---|
| 1 | MAKRFYSAEE AAAHCMASSS EEFSGSDSEY VPPASESDSS TEESWCSSST VSALEEPMEV |
| 61 | DEDVDDLEDQ EAGDRADAAA GGEPAWGPPC NFPPEIPPFT TVPGVKVDTS NFEPINFFQL |
| 121 | FMTEAILQDM VLYTNVYAEQ YLTQNPLPRY ARAHAWHPTD IAEMKRFVGL TLAMGLIKAN |
| 181 | SLESYWDTTT VLKIPVFSAT MSRNRYQLLL RFLHFNNNAT AVPPDQPGHD RLHKLRPLID |
| 241 | SLSERFAAVY TPCQNICIDE SLLLFKGRLQ FRQYIPSKRA RYGIKFYKLC ESSSGYTSYF |
| 301 | LIYEGKDSKL DPPGCPPDLT VSGKIVWELI SPLLGQGFHL YVDNFYSSIP LFTALYCLNT |
| 361 | PACGTINRNR KGLPRALLDK KLNRGETYAL RKNELLAIKF FDKKNVFMLT SIHDESVIRE |
| 421 | QRVGRPPKNK PLCSKEYSKY MGGVDRTDQL QHYYNATRKT RHWYKKVGIY LIQMALRNSY |
| 481 | IVYKAAVPGP KLSYYKYQLQ ILPALLFGGV EEQTVPEMPP SDNVARLIGK HFIDTLPPTP |
| 541 | GKQRPQKGCK VCRKRGIRRD TRYYCPKCPR NPGLCFKPCF EIYHTQLHY. |

In certain embodiments, the hyperactive piggyBac or piggyBac-like transposase comprises a sequence of:

| (SEQ ID NO: 14627) | |
|---|---|
| 1 | MAKRFYSAEE AAAHCMASSS EQTSGSDSEY VPPASESDSS TEESWCSSST VSALEEPMEV |
| 61 | DEDVDDLEDQ EAGDRADAAA GGEPAWGPPC NFPPEIPPFT TVPGVKVDTS NFEPINFFQL |
| 121 | FMTEAILQDM VLYTNVYAEQ YLTQNPLTRY ARAHAWHPTD IAEMKRFVGL TLAMGLIKAN |
| 181 | SIESYWDTTT VLSIPVFGAT MSRNRYQLLL RFLHFNNNAT AVPPDQPGHD RLHKLRPLID |
| 241 | SLSERFANVY TPCQNICIDE SLLLFKGRLQ FRQYIPSKRA RYGIKFYKLC ESSSGYTSYF |
| 301 | LIYEGKDSKL DPPGCPPDLT VSGKIVWELI SPLLGQGFHL YVDNFYSSIP LFTALYCLNT |
| 361 | PACGTINRNR KGLPRALLDK KLNRGETYAL RKNELLAIKF FDKKNVFMLT SIHDESVIRE |
| 421 | QRVGRKPKNK PLCSKEYSKY MGGVDRTDQL QHYYNATRKT RHWYKKVGIY LIQMALRNSY |
| 481 | IVYKAAVPGP KLSYYKYQLQ ILPALLFGGV EEQTVPEMPP SDNVARLIGK HFIDTLPPTP |
| 541 | GKQRPQKGCK VCRKRGIRRD TRYYCPKCPR NPGLCRKPCF EIYHTQLHY. |

In certain embodiments, the hyperactive piggyBac or piggyBac-like transposase comprises a sequence of:

| (SEQ ID NO: 14628) | |
|---|---|
| 1 | MAKRFYSAEE AAAHCSASSS EEFSGSDSEY VPPASESDSS TEESWCSSST VSALEEPMEV |
| 61 | DEDVDDLEDQ EAGDRADAAA GGEPAWGPPC NFPPEIPPFT TVPGVKVDTS NFEPINFFQL |
| 121 | FMTEAILQDM VLYTNVYAEQ YLTQNPLTRG ARAHAWHPTD IAEMKRFVGL TLAMGLIKAN |
| 181 | SLESYWDTTT VLSIPVFGAT MSRNRYQLLL RFLHFNNNAT AVPPDQPGHD RLHKLRPLID |
| 241 | SLSERFANVY TPCQNICIDE SLMLFKGRLQ FRQYIPSKRA RYGIKFYKLC ESSTGYTSYF |
| 301 | LIYEGKDSKL DPPGCPPDLT VSGKIVWELI SPLLGQGFHL YVDNFYSSIP LFTALYCLNT |
| 361 | PACGTINRNR KGLPRALLDK KLNRGETYAL RKNELLAIKF FDKKNVFMLT SIHDESVIRE |
| 421 | QRVGRPPKNK PLCSKEYSKY MGGVDRTDQL QHYYNATRKT RHWYKKVGIY LIQMALRNSY |
| 481 | IVYKAAVPGP KLSYYKYQLQ ILPALLFGGV EEQTVPEMPP SDNVARLIGK HFIDTLPPTP |
| 541 | GKQRPQKGCK VCRKRGIRRD TRYYCPKCPR NPGLCRKPCF EIYHTQLHY. |

In certain embodiments, the hyperactive piggyBac or piggyBac-like transposase comprises a sequence of:

| (SEQ ID NO: 149) | |
|---|---|
| 1 | MAKRFYSAEE AAAHCMASSS EEFSGSDSEY VPPASESDSS TEESWCSSST VSALEEPMEV |
| 61 | DEDVDDLEDQ EAGDRADAAA GGEPAWGPPC NFPPEIPPFT TVPGVKVDTS NFEPINFFQL |
| 121 | FMTEAILQDM VLYTNVYAEQ YLTQNPLTRY ARAHAWHPTD IAEMKRFVGL TLAMGLIKAN |
| 181 | SLESYWDTTT VLSIPVFGAT MSRNRYQLLL RFLHFNNNAT AVPPDQPGHD RLHKLRPLID |
| 241 | SLSERFANVY TPCQNICIDE SLLLFKGRLQ FRQYIPSKRA RYGIKFYKLC ESSSGYTSYF |
| 301 | LIYEGKDSKL DPPGCPPDLT VSGKIVWELI SPLLGQGFHL YVDNFYSSIP LFTALYCLNT |
| 361 | PACGTINRNR KGLPRALLDK KLNRGETYAL RKNELLAIKF FDKKNVFMLT SIHDESVIRE |
| 421 | QRVGRPPKNK PLCSKEYSKY MGGVDRTDQL QHYYNATRKT RHWYKKVGIY LIQMALRNSY |
| 481 | IVYKAAVPGP KLSYYKYQLQ ILPALLFGGV EEQTVPEMPP SDNVARLIGK HFIDTLPPTP |
| 541 | GKQRPQKGCK VCRKRGIRRD TRYYCPKCPR NPGLCRKPCF EIYHTQLHY. |

In certain embodiments, the hyperactive piggyBac or piggyBac-like transposase comprises an amino acid substitution at a position selected from amino acid 6, 7, 16, 19, 20, 21, 22, 23, 24, 26, 28, 31, 34, 67, 73, 76, 77, 88, 91, 141, 145, 146, 148, 150, 157, 162, 179, 182, 189, 192, 193, 196, 198, 200, 210, 212, 218, 248, 263, 270, 294, 297, 308, 310, 333, 336, 354, 357, 358, 359, 377, 423, 426, 428, 438, 447, 450, 462, 469, 472, 498, 502, 517, 520, 523, 533, 534, 576, 577, 582, 583 or 587 (relative to SEQ ID NO: 14517). In certain embodiments, the hyperactive piggyBac or piggyBac-like transposase comprises an amino acid substitution of Y6C, S7G, M165, S19G, 520Q, 520G, 520D, E21D, E22Q, F23T, F23P, S24Y, S26V, S28Q, V31K, A34E, L67A, G73H, A76V, D77N, P88A, N91D, Y141Q, Y141A, N145E, N145V, P146T, P146V, P146K, P148T, P148H, Y150G, Y1505, Y150C, H157Y, A162C, A179K, L182I, L182V, T189G, L192H, S193N, S193K, V196I, S198G, T200W, L210H, F212N, N218E, A248N, L263M, Q270L, S294T, T297M, 5308R, L310R, L333M, Q336M, A354H, C357V, L358F, D359N, L377I, V423H, P426K, K428R, S438A, T447G, T447A, L450V, A462H, A462Q, I469V, I472L, Q498M, L502V, E5171, P520D, P520G, N523S, 1533E, D534A, F576R, F576E, K5771, I582R, Y583F, L587Y or L587W, or any combination thereof including at least 1, 2, 3, 4, 5, 6, 7, 8, 9, 10 or all of these mutations (relative to SEQ ID NO: 14517).

In certain embodiments, the hyperactive piggyBac or piggyBac-like transposase comprises one or more substitutions of an amino acid that is not wild type, wherein the one or more substitutions a for wild type amino acid comprises a substitution of A2X, K3X, R4X, FSX, Y6X, S7X, A11X, A13X, C15X, M16X, A17X, 518X, 519X, 520X, E21X, E22X, F23X, S24X, G25X, 26X, D27X, S28X, E29X, E42X, E43X, S44X, C46X, S47X, S48X, S49X, T50X, V51X, S52X, A53X, L54X, E55X, E56X, P57X, M58X, E59X, E62X, D63X, V64X, D65X, D66X, L67X, E68X, D69X, Q70X, E71X, A72X, G73X, D74X, R75X, A76X, D77X, A78X, A79X, A80X, G81X, G82X, E83X, P84X, A85X, W86X, G87X, P88X, P89X, C90X, N91X, F92X, P93X, E95X, I96X, P97X, P98X, F99X, T100X, T101X, P103X, G104X, V105X, K106X, V107X, D108X, T109X, N111X, P114X, Il 15X, N116X, F117X, F118X, Q119X, M122X, T123X, E124X, A125X, I126X, L127X, Q128X, D129X, M130X, L132X, Y133X, V126X, Y127X, A138X, E139X, Q140X, Y141X, L142X, Q144X, N145X, P146X, L147X, P148X, Y150X, A151X, A155X, H157X, P158X, I161X, A162X, V168X, T171X, L172X, A173X, M174X, I177X, A179X, L182X, D187X, T188X, T189X, T190X, L192X, S193X, I194X, P195X, V196X, S198X, A199X, T200X, S202X, L208X, L209X, L210X, R211X, F212X, F215X, N217X, N218X, A219X, T220X, A221X, V222X, P224X, D225X, Q226X, P227X, H229X, R231X, H233X, L235X, P237X, I239X, D240X, L242X, S243X, E244X, R244X, F246X, A247X, A248X, V249X, Y250X, T251X, P252X, C253X, Q254X, I256X, C257X, I258X, D259X, E260X, S261X, L262X, L263X, L264X, F265X, K266X, G267X, R268X, L269X, Q270X, F271X, R272X, Q273X, Y274X, I275X, P276X, S277X, K278X, R279X, A280X, R281X, Y282X, G283X, I284X, K285X, F286X, Y287X, K288X, L289X, C290X, E291X, S292X, S293XS294X, G295X, Y296X, T297X, S298X, Y299X, F300X, E304X, L310X, P313X, G314X, P316X, P317X, D318X, L319X, T320X, V321X, K324X, E328X, I330X, S331X, P332X, L333X, L334X, G335X, Q336X, F338X, L340X, D343X, N344X, F345X, Y346X, S347X, L351X, F352X, A354X, L355X, Y356X, C357X, L358X, D359X, T360X, R422X, Y423X, G424X, P426X, K428X, N429X, K430X, P431X, L432X, S434X, K435X, E436X, S438X, K439X, Y440X, G443X, R446X, T447X, L450X, Q451X, N455X, T460X, R461X, A462X, K465X, V467X, G468X, I469X, Y470X, L471X, I472X, M474X, A475X, L476X, R477X, S479X, Y480X, V482XY483X, K484X, A485X, A486X, V487X, P488X, P490X, K491X, S493X, Y494X, Y495X, K496X, Y497T, Q498X, L499X, Q500X, I501X, L502X, P503X, A504X, L505X, L506X, F507X, G508X, G509X, V510X, E511X, E512X, Q513X, T514X, V515X, E517X, M518X, P519X, P520X, S521X, D522X, N523X, V524X, A525X, L527X, I528X, K530X, H531X, F532X, I533X, D534X, T535X, L536X, T539X, P540X, Q546X, K550X, R553X, K554X, R555X, G556X, I557X, R558X, R559X, D560X, T561X, Y564X, P566X, K567X, P569X, R570X, N571X, L574X, C575X, F576X, K577X, P578X, F580X, E581X, I582X, Y583X, T585X, Q586X, L587X, H588X or Y589X (relative to SEQ ID NO: 14517). A list of hyperactive amino acid substitutions can be found in U.S. Pat. No. 10,041,077, the contents of which are incorporated by reference in their entirety.

In certain embodiments, the piggyBac or piggyBac-like transposase is integration deficient. In certain embodiments, an integration deficient piggyBac or piggyBac-like transposase is a transposase that can excise its corresponding transposon, but that integrates the excised transposon at a lower frequency than a corresponding naturally occurring transposase. In certain embodiments, the piggyBac or piggyBac-like transposase is an integration deficient variant of SEQ ID NO: 14517. In certain embodiments, the integration deficient piggyBac or piggyBac-like transposase is deficient relative to SEQ ID NO: 14517.

In certain embodiments, the piggyBac or piggyBac-like transposase is active for excision but deficient in integration. In certain embodiments, the integration deficient piggyBac or piggyBac-like transposase comprises a sequence that is at least 90% identical to a sequence of:

| (SEQ ID NO: 14605) | |
|---|---|
| 1 | MAKRFYSAEE AAAHCMASSS EEFSGSDSEY VPPASESDSS TEESWCSSST VSALEEPMEV |
| 61 | DEDVDDLEDQ EAGDRVDAAA GGEPAWGPPC NFPPEIPPFT TVPGVKVDTS NFEPINFFQL |
| 121 | FMTEAILQDM VLYTNVYAEQ YLTQNPLPRY ARAHAWHPTD IAEMKRFVGL TLAMGLIKAN |
| 181 | SLESYWDTTT VLSIPVFSAT MSRNRYQLLL KFLHFNNEAT AVPPDQPGHD RLHKLRPLID |
| 241 | SLSERFAAVY TPCQNICIDE SLLLFKGRLQ FRQYIPSKRA RYGIKFYKLC ESSSGYTSYF |
| 301 | LIYEGKDSKL DPPGCPPDLT VSGKIVWELI SPLLGQGFHL YVDNFYSSIP LFTALYCLDT |
| 361 | PACGTINRNR KGLPRALLDK KLNRGETYAL RKNELLAIKF FDKKNVFMLT SIHDESVIRE |
| 421 | QRVGRPPKNK PLCSKEYSKY MGGVDRTDQL QHYYNATRKT RAWYKKVGIY LIQMALRNSY |
| 481 | IVYKAAVPGP KLSYYKYQLQ ILPALLFGGV EEQTVPEMPP SDNVARLIGK HFIDTLPPTP |
| 541 | GKQRPQKGCK VCRKRGIRRD TRYYCPKCPR NPGLCFKPCF EIYHTQLHYG RR. |

In certain embodiments, the integration deficient piggyBac or piggyBac-like transposase comprises a sequence that is at least 90% identical to a sequence of:

| (SEQ ID NO: 14604) | |
|---|---|
| 1 | MAKRFYSAEE AAAHCMASSS EEFSGSDSEY VPPASESDSS TEESWCSSST VSALEEPMEV |
| 61 | DEDVDDLEDQ EAGDRADAAA GGEPAWGPPC NFPPEIPPFT TVPGVKVDTS NFEPINFFQL |
| 121 | FMTEAILQDM VLYTNVYAEQ YLTQVPLPRY ARAHAWHPTD IAEMKRFVGL TLAMGLIKAN |
| 181 | SLESYWDTTT VLNIPVFSAT MSRNRYQLLL RFLEFNNEAT AVPPDQPGHD RLHKLRPLID |
| 241 | SLSERFAAVY TPCQNICIDE SLLLFKGRLQ FRQYIPSKRA RYGIKFYKLC ESSSGYTSYF |
| 301 | LIYEGKDSKL DPPGCPPDLT VSGKIVWELI SPLLGQGFHL YVDNFYSSIP LFTALYCLDT |
| 361 | PACGTINRNR KGLPRALLDK KLNRGETYAL RKNELLAIKF FDKKNVFMLT SIHDESVIRE |
| 421 | QRVGRPPKNK PLCSKEYSKY MGGVDRTDQL QHYYNATRKT RAWYKKVGIY LIQMALRNSY |
| 481 | IVYKAAVPGP KLSYYKYQLQ ILPALLFGGV EEQTVPEMPP SDNVARLIGK HFIDTLPPTP |
| 541 | GKQRPQKGCK VCRKRGIRRD TRYYCPKCPR NPGLCFKPCF EIYHTQLHY. |

In certain embodiments, the integration deficient piggyBac or piggyBac-like transposase comprises a sequence that is at least 90% identical to a sequence of:

| (SEQ ID NO: 14611) | |
|---|---|
| 1 | MAKRFYSAEE AAAHCMASSS EEFSGSDSEY VPPASESDSS TEESWCSSST VSALEEPMEV |
| 61 | DEDVDDLEDQ EAGDRADAAA GGEPAWGPPC NFPPEIPPFT TVPGVKVDTS NFEPINFFQL |
| 121 | FMTEAILQDM VLYTNVYAEQ YLTQNVLPRY ARAHAWHPTD IAEMKRFVGL TLAMGLIKAN |
| 181 | SLESYWDTTT VLSIPVFSAT MSRNRYQLLL RFLHFNNDAT AVPPDQPGHD RLHKLRPLID |
| 241 | SLTERFAAVY TPCQNICIDE SLLLFKGRLQ FRQYIPSKRA RYGIKFYKLC ESSSGYTSYF |
| 301 | LIYEGKDSKL DPPGCPPDLT VSGKIVWELI SPLLGQGFHL YVDNFYSSIP LFTALYCLDT |
| 361 | PACGTINRNR KGLPRALLDK KLNRGETYAL RKNELLAIKF FDKKNVFMLT SIHDESVIRE |
| 421 | QRVGRPPKNK PLCSKEYSKY MGGVDRTDQL QHYYNATRKT RAWYKKVGIY LIQMALRNSY |
| 481 | IVYKAAVPGP KLSYYKYQLQ ILPALLFGGV EEQTVPEMPP SDNVARLIGK HFIDTLPPTP |
| 541 | GKQRPQKGCK VCRKRGIRRD TRYYCPKCPR NPGLCFKPCF EIYHTQLHYG RR. |

In certain embodiments, the integration deficient piggyBac or piggyBac-like transposase comprises SEQ ID NO: 14611. In certain embodiments, the integration deficient piggyBac or piggyBac-like transposase comprises a sequence that is at least 90% identical to a sequence of:

| (SEQ ID NO: 14612) | |
|---|---|
| 1 | MAKRFYSAEE AAAHCMASSS EEFSGSDSEY VPPASESDSS TEESWCSSST VSALEEPMEV |
| 61 | DEDVDDLEDQ EAGDRADAAP GGEPAWGPPC NFPPEIPPFT TVPGVKVDTS NFEPINFFQL |
| 121 | FMTEAILQDM VLYTNVYAEQ YLTQVPLPRY ARAHAWHPTD IAEMKRFVGL TLAMGLIKAN |
| 181 | SLESYWDTTT VLSIPVFSAT MSRNRYQLLL RFLHFNNEAT AVPPDQPGHD RLHKLRPLID |
| 241 | SLSERFAAVY TPCQNICIDE SLLLFKGRLQ FRQYIPSKRA RYGIKFYKLC ESSSGYTSYF |
| 301 | LIYEGKDSKL DPPGCPPDLT VSGKIVWELI SPLLGQGFHL YVDNFYSSIP LFTALYCLDT |
| 361 | PACGTINRNR KGLPRALLDK KLNRGETYAL RKNELLAIKF FDKKNVFMLT SIHDESVIRE |
| 421 | QRVGRPPKNK PLCSKEYSKY MGGVDRTDQL QHYYNATRKT RAWYKKVGIY LIQMALRNSY |
| 481 | IVYKAAVPGP KLSYYKYQLQ ILPALLFGGV EEQTVPEMPP SDNVARLIGK HFIDTLPPTP |
| 541 | GKQRPQKGCK VCRKRGIRRD TRYYCPKCPR NPGLCFKPCF EIYHTQLHYG RR. |

In certain embodiments, the integration deficient piggyBac or piggyBac-like transposase comprises SEQ ID NO: 14612. In certain embodiments, the integration deficient piggyBac or piggyBac-like transposase comprises a sequence that is at least 90% identical to a sequence of:

| (SEQ ID NO: 14613) | |
|---|---|
| 1 | MAKRFYSAEE AAAHCMASSS EEFSGSDSEY VPPASESDSS TEESWCSSST VSALEEPMEV |
| 61 | DEDVDDLEDQ EAGDRADAAA GGEPAWGPPC NFPPEIPPFT TVPGVKVDTS NFEPINFFQL |
| 121 | FMTEAILQDM VLYTNVYAEQ YLTQVPLPRY ARAHAWHPTD IAEMKRFVGL TLAMGLIKAN |
| 181 | SLESYWDTTT VLNIPVFSAT MSRNRYQLLL RFLEFNNNAT AVPPDQPGHD RLHKLRPLID |
| 241 | SLSERFAAVY TPCQNICIDE SLLLFKGRLQ FRQYIPSKRA RYGIKFYKLC ESSSGYTSYF |
| 301 | LIYEGKDSKL DPPGCPPDLT VSGKIVWELI SPLLGQGFHL YVDNFYSSIP LFTALYCLDT |
| 361 | PACGTINRNR KGLPRALLDK KLNRGETYAL RKNELLAIKF FDKKNVFMLT SIHDESVIRE |
| 421 | QRVGRPPKNK PLCSKEYSKY MGGVDRTDQL QHYYNATRKT RAWYKKVGIY LIQMALRNSY |
| 481 | IVYKAAVPGP KLSYYKYQLQ ILPALLFGGV EEQTVPEMPP SDNVARLIGK HFIDTLPPTP |
| 541 | GKQRPQKGCK VCRKRGIRRD TRYYCPKCPR NPGLCFKPCF EIYHTQLHYG RR. |

In certain embodiments, the integration deficient piggyBac or piggyBac-like transposase comprises SEQ ID NO: 14613. In certain embodiments, the integration deficient piggyBac or piggyBac-like transposase comprises an amino acid substitution wherein the Asn at position 218 is replaced by a Glu or an Asp (N218D or N218E) (relative to SEQ ID NO: 14517).

In certain embodiments, the excision competent, integration deficient piggyBac or piggyBac-like transposase comprises one or more substitutions of an amino acid that is not wild type, wherein the one or more substitutions a for wild type amino acid comprises a substitution of A2X, K3X, R4X, FSX, Y6X, S7X, ABX, E9X, E10X, A11X, A12X, A13X, H14X, C15X, M16X, A17X, 518X, 519X, 520X, E21X, E22X, F23X, S24X, G25X, 26X, D27X, S28X, E29X, V31X, P32X, P33X, A34X, 535X, E36X, S37X, D38X, S39X, 540X, T41X, E42X, E43X, S44X, W45X, C46X, S47X, S48X, S49X, T50X, V51X, S52X, A53X, L54X, E55X, E56X, P57X, M58X, E59X, V60X, M122X, T123X, E124X, A125X, L127X, Q128X, D129X, L132X, Y133X, V126X, Y127X, E139X, Q140X, Y141X, L142X, T143X, Q144X, N145X, P146X, L147X, P148X, R149X, Y150X, A151X, H154X, H157X, P158X, T159X, D160X, I161X, A162X, E163X, M164X, K165X, R166X, F167X, V168X, G169X, L170X, T171X, L172X, A173X, M174X, G175X, L176X, I177X, K178X, A179X, N180X, 5181X, L182X, 5184X, Y185X, D187X, T188X, T189X, T190X, V191X, L192X, 5193X, I194X, P195X, V196X, F197X, 5198X, A199X, T200X, M201X, 5202X, R203X, N204X, R205X, Y206X, Q207X, L208X, L209X, L210X, R211X, F212X, L213X, H241X, F215X, N216X, N217X, N218X, A219X, T220X, A221X, V222X, P223X, P224X, D225X, Q226X, P227X, G228X, H229X, D230X, R231X, H233X, K234X, L235X, R236X, L238X, I239X, D240X, L242X, S243X, E244X, R244X, F246X, A247X, A248X, V249X, Y250X, T251X, P252X, C253X, Q254X, N255X, I256X, C257X, I258X, D259X, E260X, S261X, L262X, L263X, L264X, F265X, K266X, G267X, R268X, L269X, Q270X, F271X, R272X, Q273X, Y274X, I275X, P276X, S277X, K278X, R279X, A280X, R281X, Y282X, G283X, I284X, K285X, F286X, Y287X, K288X, L289X, C290X, E291X, S292X, S293X, S294X, G295X, Y296X, T297X, S298X, Y299X, F300X, I302X, E304X, G305X, K306X, D307X, S308X, K309X, L310X, D311X, P312X, P313X, G314X, C315X, P316X, P317X, D318X, L319X, T320X, V321X, S322X, G323X, K324X, I325X, V326X, W327X, E328X, L329X, 1330X, S331X, P332X, L333X, L334X, G335X, Q336X, F338X, H339X, L340X, V342X, N344X, F345X, Y346X, S347X, S348X, I349X, L351X, T353X, A354X, Y356X, C357X, L358X, D359X, T360X, P361X, A362X, C363X, G364X, I366X, N367X, R368X, D369X, K371X, G372X, L373X, R375X, A376X, L377X, L378X, D379X, K380X, K381X, L382X, N383X, R384XG385X, T387X, Y388X, A389X, L390X, K392X, N393X, E394X, A397X, K399X, F400X, F401X, D402X, N405X, L406X, L409X, R422X, Y423X, G424X, E425X, P426X, K428X, N429X, K430X, P431X, L432X, S434X, K435X, E436X, S438X, K439X, Y440X, G442X, G443X, V444X, R446X, T447X, L450X, Q451X, H452X, N455X, T457X, R458X, T460X, R461X, A462X, Y464X, K465X, V467X, G468X, I469X, L471X, I472X, Q473X, M474X, L476X, R477X, N478X, S479X, Y480X, V482XY483X, K484X, A485X, A486X, V487X, P488X, G489X, P490X, K491X, L492X, S493X, Y494X, Y495X, K496X, Q498X, L499X, Q500X, I501X, L502X, P503X, A504X, L505X, L506X, F507X, G508X, G509X, V510X, E511X, E512X, Q513X, T514X, V515X, E517X, M518X, P519X, P520X, S521X, D522X, N523X, V524X, A525X, L527X, I528X, G529X, K530X, F532X, I533X, D534X, T535X, L536X, P537X, P538X, T539X, P540X, G541X, F542X, Q543X, R544X, P545X, Q546X, K547X, G548X, C549X, K550X, V551X, C552X, R553X, K554X, R555X, G556X, I557X, R558X, R559X, D560X, T561X, R562X, Y563X, Y564X, C565X, P566X, K567X, C568X, P569X, R570X, N571X, P572X, G573X, L574X, C575X, F576X, K577X, P578X, C579X, F580X, E581X, I582X, Y583X, H584X, T585X, Q586X, L587X, H588X or Y589X (relative to SEQ ID NO: 14517). A list of excision competent, integration deficient amino acid substitutions can be found in U.S. Pat. No. 10,041,077, the contents of which are incorporated by reference in their entirety.

In certain embodiments, the piggyBac or piggyBac-like transposase is fused to a nuclear localization signal. In certain embodiments, SEQ ID NO: 14517 or SEQ ID NO: 14518 is fused to a nuclear localization signal. In certain embodiments, the amino acid sequence of the piggyBac or piggyBac like transposase fused to a nuclear localization signal is encoded by a polynucleotide sequence comprising:

| (SEQ ID NO: 14626) | |
|---|---|
| 1 | atggcaccca aaaagaaacg taaagtgatg gccaaaagat tttacagcgc cgaagaagca |
| 61 | gcagcacatt gcatggcatc gtcatccgaa gaattctcgg ggagcgattc cgaatatgtc |
| 121 | ccaccggcct cggaaagcga ttcgagcact gaggagtcgt ggtgttcctc ctcaactgtc |
| 181 | tcggctcttg aggagccgat ggaagtggat gaggatgtgg acgacttgga ggaccaggaa |
| 241 | gccggagaca gggccgacgc tgccgcggga ggggagccgg cgtggggacc tccatgcaat |
| 301 | tttcctcccg aaatcccacc gttcactact gtgccgggag tgaaggtcga cacgtccaac |
| 361 | ttcgaaccga tcaatttctt tcaactcttc atgactgaag cgatcctgca agatatggtg |
| 421 | ctctacacta atgtgtacgc cgagcagtac ctgactcaaa acccgctgcc tcgctacgcg |
| 481 | agagcgcatg cgtggcaccc gaccgatatc gcggagatga agcggttcgt gggactgacc |
| 541 | ctcgcaatgg gcctgatcaa ggccaacagc ctcgagtcat actgggatac cacgactgtg |
| 601 | cttagcattc cggtgttctc cgctaccatg tcccgtaacc gctaccaact cctgctgcgg |
| 661 | ttcctccact tcaacaacaa tgcgaccgct gtgccacctg accagccagg acacgacaga |
| 721 | ctccacaagc tgcggccatt gatcgactcg ctgagcgagc gattcgccgc ggtgtacacc |
| 781 | ccttgccaaa acatttgcat cgacgagtcg cttctgctgt ttaaaggccg gcttcagttc |
| 841 | cgccagtaca tcccatcgaa gcgcgctcgc tatggtatca aattctacaa actctgcgag |
| 901 | tcgtccagcg gctacacgtc atacttcttg atctacgagg ggaaggactc taagctggac |
| 961 | ccaccggggt gtccaccgga tcttactgtc tccggaaaaa tcgtgtggga actcatctca |
| 1021 | cctctcctcg gacaaggctt tcatctctac gtcgacaatt tctactcatc gatccctctg |
| 1081 | ttcaccgccc tctactgcct ggatactcca gcctgtggga ccattaacag aaaccggaag |
| 1141 | ggtctgccga gagcactgct ggataagaag ttgaacaggg gagagactta cgcgctgaga |
| 1201 | aagaacgaac tcctcgccat caaattcttc gacaagaaaa atgtgtttat gctcacctcc |
| 1261 | atccacgacg aatccgtcat ccgggagcag cgcgtgggca ggccgccgaa aaacaagccg |
| 1321 | ctgtgctcta aggaatactc caagtacatg gggggtgtcg accggaccga tcagctgcag |
| 1381 | cattactaca acgccactag aaagacccgg gcctggtaca agaaagtcgg catctacctg |
| 1441 | atccaaatgg cactgaggaa ttcgtatatt gtctacaagg ctgccgttcc gggcccgaaa |
| 1501 | ctgtcatact acaagtacca gcttcaaatc ctgccggcgc tgctgttcgg tggagtggaa |
| 1561 | gaacagactg tgcccgagat gccgccatcc gacaacgtgg cccggttgat cggaaagcac |
| 1621 | ttcattgata ccctgcctcc gacgcctgga aagcagcggc cacagaaggg atgcaaagtt |
| 1681 | tgccgcaagc gcggaatacg gcgcgatacc cgctactatt gcccgaagtg cccccgcaat |
| 1741 | cccggactgt gtttcaagcc ctgttttgaa atctaccaca cccagttgca ttac. |

In certain embodiments, the piggyBac or piggyBac-like transposon is isolated or derived from Xenopus tropicalis. In certain embodiments, the piggyBac or piggyBac-like transposon comprises a sequence of:

| (SEQ ID NO: 14519) | |
|---|---|
| 1 | ttaacctttt tactgccaat gacgcatggg atacgtcgtg gcagtaaaag ggcttaaatg |
| 61 | ccaacgacgc gtcccatacg ttgttggcat tttaagtctt ctctctgcag cggcagcatg |
| 121 | tgccgccgct gcagagagtt tctagcgatg acagcccctc tgggcaacga gccggggggg |
| 181 | ctgtc. |

In certain embodiments, the piggyBac or piggyBac-like transposon comprises a sequence of:

| (SEQ ID NO: 14520) | |
|---|---|
| 1 | tttgcatttt tagacattta gaagcctata tcttgttaca gaattggaat tacacaaaaa |
| 61 | ttctaccata ttttgaaagc ttaggttgtt ctgaaaaaaa caatatattg ttttcctggg |
| 121 | taaactaaaa gtcccctcga ggaaaggccc ctaaagtgaa acagtgcaaa acgttcaaaa |
| 181 | actgtctggc aatacaagtt ccactttgac caaaacggct ggcagtaaaa gggttaa. |

In certain embodiments, the piggyBac or piggyBac-like transposon comprises SEQ ID NO: 14519 and SEQ ID NO: 14520. In certain embodiments, the piggyBac or piggyBac-like transposon comprises a sequence of:

| (SEQ ID NO: 14521) | |
|---|---|
| 1 | ttaacccttt gcctgccaat cacgcatggg atacgtcgtg gcagtaaaag ggcttaaatg |
| 61 | ccaacgacgc gtcccatacg ttgttggcat tttaagtctt ctctctgcag cggcagcatg |
| 121 | tgccgccgct gcagagagtt tctagcgatg acagcccctc tgggcaacga gccggggggg |
| 181 | ctgtc. |

In certain embodiments, the piggyBac or piggyBac-like transposon comprises a sequence of:

| (SEQ ID NO: 14522) | |
|---|---|
| 1 | tttgcatttt tagacattta gaagcctata tcttgttaca gaattggaat tacacaaaaa |
| 61 | ttctaccata ttttgaaagc ttaggttgtt ctgaaaaaaa caatatattg ttttcctggg |
| 121 | taaactaaaa gtcccctcga ggaaaggccc ctaaagtgaa acagtgcaaa acgttcaaaa |
| 181 | actgtctggc aatacaagtt ccactttggg acaaatcggc tggcagtgaa agggttaa. |

In certain embodiments, the piggyBac or piggyBac-like transposon comprises a sequence of:

| (SEQ ID NO: 14523) | |
|---|---|
| 1 | ttaacctttt tactgccaat gacgcatggg atacgtcgtg gcagtaaaag ggcttaaatg |
| 61 | ccaacgacgc gtcccatacg ttgttggcat tttaattctt ctctctgcag cggcagcatg |
| 121 | tgccgccgct gcagagagtt tctagcgatg acagcccctc tgggcaacga gccggggggg |
| 181 | ctgtc. |

In certain embodiments, the piggyBac or piggyBac-like transposon comprises SEQ ID NO: 14520 and SEQ ID NO: 14519, SEQ ID NO: 14521 or SEQ ID NO: 14523. In certain embodiments, the piggyBac or piggyBac-like transposon comprises SEQ ID NO: 14522 and SEQ ID NO: 14519, SEQ ID NO: 14521 or SEQ ID NO: 14523. In certain embodiments, the piggyBac or piggyBac-like transposon comprises one end comprising at least 14, 16, 18, 20, 30 or 40 contiguous nucleotides from SEQ ID NO: 14519, SEQ ID NO: 14521 or SEQ ID NO: 14523. In certain embodiments, the piggyBac or piggyBac-like transposon comprises one end comprising at least 14, 16, 18, 20, 30 or 40 contiguous nucleotides from SEQ ID NO: 14520 or SEQ ID NO: 14522. In certain embodiments, the piggyBac or piggyBac-like transposon comprises one end with at least 90% identity to SEQ ID NO: 14519, SEQ ID NO: 14521 or SEQ ID NO: 14523. In certain embodiments, the piggyBac or piggyBac-like transposon comprises one end with at least 90% identity to SEQ ID NO: 14520 or SEQ ID NO: 14522. In one embodiment, one transposon end is at least 90% identical to SEQ ID NO: 14519 and the other transposon end is at least 90% identical to SEQ ID NO: 14520.

In certain embodiments, the piggyBac or piggyBac-like transposon comprises a sequence of TTAACCTTTTTACTGCCA (SEQ ID NO: 14524). In certain embodiments, the piggyBac or piggyBac-like transposon comprises a sequence of TTAACCCTTTGCCTGCCA (SEQ ID NO: 14526). In certain embodiments, the piggyBac or piggyBac-like transposon comprises a sequence of TTAACCYTTTTACTGCCA (SEQ ID NO: 14527). In certain embodiments, the piggyBac or piggyBac-like transposon comprises a sequence of TGGCAGTAAAAGGGTTAA (SEQ ID NO: 14529). In certain embodiments, the piggyBac or piggyBac-like transposon comprises a sequence of TGGCAGTGAAAGGGTTAA (SEQ ID NO: 14531). In certain embodiments, the piggyBac or piggyBac-like transposon comprises a sequence of TTAACCYTTTKMCTGCCA (SEQ ID NO: 14533). In certain embodiments, one end of the piggyBac or piggyBac-like transposon comprises a sequence selected from SEQ ID NO: 14524, SEQ ID NO: 14526 and SEQ ID NO: 14527. In certain embodiments, one end of the piggyBac™ (PB) or piggyBac-like transposon comprises a sequence selected from SEQ ID NO: 14529 and SEQ ID NO: 14531. In certain embodiments, each inverted terminal repeat of the piggyBac or piggyBac-like transposon comprises a sequence of ITR sequence of CCYTTTKMCTGCCA (SEQ ID NO: 14563). In certain embodiments, each end of the piggyBac™ (PB) or piggyBac-like transposon comprises SEQ ID NO: 14563 in inverted orientations. In certain embodiments, one ITR of the piggyBac or piggyBac-like transposon comprises a sequence selected from SEQ ID NO: 14524, SEQ ID NO: 14526 and SEQ ID NO: 14527. In certain embodiments, one ITR of the piggyBac or piggyBac-like transposon comprises a sequence selected from SEQ ID NO: 14529 and SEQ ID NO: 14531. In certain embodiments, the piggyBac or piggyBac like transposon comprises SEQ ID NO: 14533 in inverted orientation in the two transposon ends.

In certain embodiments, The piggyBac or piggyBac-like transposon may have ends comprising SEQ ID NO: 14519 and SEQ ID NO: 14520 or a variant of either or both of these having at least 90% sequence identity to SEQ ID NO: 14519 or SEQ ID NO: 14520, and the piggyBac or piggyBac-like transposase has the sequence of SEQ ID NO: 14517 or a variant showing at least %, 10%, 15%, 20%, 25%, 30%, 35%, 40%, 45%, 50%, 55%, 60%, 65%, 70%, 75%, 80%, 85%, 90%, 95%, 99% or any percentage in between sequence identity to SEQ ID NO: 14517 or SEQ ID NO: 14518. In certain embodiments, one piggyBac or piggyBac-like transposon end comprises at least 14 contiguous nucleotides from SEQ ID NO: 14519, SEQ ID NO: 14521 or SEQ ID NO: 14523, and the other transposon end comprises at least 14 contiguous nucleotides from SEQ ID NO: 14520 or SEQ ID NO: 14522. In certain embodiments, one transposon end comprises at least 15, at least 16, at least 17, at least 18, at least 19, at least 20, at least 22, at least 25, at least 30 contiguous nucleotides from SEQ ID NO: 14519, SEQ ID NO: 14521 or SEQ ID NO: 14523, and the other transposon end comprises at least 15, at least 16, at least 17, at least 18, at least 19, at least 20, at least 22, at least 25 or at least 30 contiguous nucleotides from SEQ ID NO: 14520 or SEQ ID NO: 14522.

In certain embodiments, the piggyBac or piggyBac-like transposase recognizes a transposon end with a left sequence corresponding to SEQ ID NO: 14519, and a right sequence corresponding to SEQ ID NO: 14520. It will excise the transposon from one DNA molecule by cutting the DNA at the 5′-TTAA-3′ sequence at the left end of one transposon end to the 5′-TTAA-3′ at the right end of the second transposon end, including any heterologous DNA that is placed between them, and insert the excised sequence into a second DNA molecule. In certain embodiments, truncated and modified versions of the left and right transposon ends will also function as part of a transposon that can be transposed by the piggyBac or piggyBac-like transposase. For example, the left transposon end can be replaced by a sequence corresponding to SEQ ID NO: 14521 or SEQ ID NO: 14523, the right transposon end can be replaced by a shorter sequence corresponding to SEQ ID NO: 14522. In certain embodiments, the left and right transposon ends share an 18 bp almost perfectly repeated sequence at their ends (5′-TTAACCYTTTKMCTGCCA: SEQ ID NO: 14533) that includes the 5′-TTAA-3′ insertion site, which sequence is inverted in the orientation in the two ends. That is in SEQ ID NO: 14519 and SEQ ID NO: 14523 the left transposon end begins with the sequence 5′-TTAACCTTTTTACTGCCA-3′ (SEQ ID NO: 14524), or in SEQ ID NO: 14521 the left transposon end begins with the sequence 5′-TTAACCCTTTGCCTGCCA-3′ (SEQ ID NO: 14526); the right transposon ends with approximately the reverse complement of this sequence: in SEQ ID NO: 14520 it ends 5′ TGGCAGTAAAAGGGTTAA-3′ (SEQ ID NO: 14529), in SEQ ID NO: 14522 it ends 5′-TGGCAGTGAAAGGGTTAA-3′ (SEQ ID NO: 14531.) One embodiment of the invention is a transposon that comprises a heterologous polynucleotide inserted between two transposon ends each comprising SEQ ID NO: 14533 in inverted orientations in the two transposon ends. In certain embodiments, one transposon end comprises a sequence selected from SEQ ID NOS: 14524, SEQ ID NO: 14526 and SEQ ID NO: 14527. In some embodiments, one transposon end comprises a sequence selected from SEQ ID NO: 14529 and SEQ ID NO: 14531.

In certain embodiments, the piggyBac™ (PB) or piggyBac-like transposon is isolated or derived from Xenopus tropicalis. In certain embodiments, the piggyBac or piggyBac-like transposon comprises at a sequence of:

| (SEQ ID NO: 14573) | |
|---|---|
| 1 | ccctttgcct gccaatcacg catgggatac gtcgtggcag taaaagggct taaatgccaa |
| 61 | cgacgcgtcc catacgtt. |

In certain embodiments, the piggyBac or piggyBac-like transposon comprises at a sequence of:

| (SEQ ID NO: 14574) | |
|---|---|
| 1 | cctgggtaaa ctaaaagtcc cctcgaggaa aggcccctaa agtgaaacag tgcaaaacgt |
| 61 | tcaaaaactg tctggcaata caagttccac tttgggacaa atcggctggc agtgaaaggg. |

In certain embodiments, the piggyBac or piggyBac-like transposon comprises at least 16 contiguous bases from SEQ ID NO: 14573 or SEQ ID NO: 14574, and inverted terminal repeat of CCYTTTBMCTGCCA (SEQ ID NO: 14575).

In certain embodiments, the piggyBac or piggyBac-like transposon comprises at a sequence of:

| (SEQ ID NO: 14579) | |
|---|---|
| 1 | ccctttgcct gccaatcacg catgggatac gtcgtggcag taaaagggct taaatgccaa |
| 61 | cgacgcgtcc catacgttgt tggcatttta agtcttctct ctgcagcggc agcatgtgcc |
| 121 | gccgctgcag agagtttcta gcgatgacag cccctctggg caacgagccg ggggggctgt |
| 181 | c. |

In certain embodiments, the piggyBac or piggyBac-like transposon comprises at a sequence of:

| (SEQ ID NO: 14580) | |
|---|---|
| 1 | cctttttact gccaatgacg catgggatac gtcgtggcag taaaagggct taaatgccaa |
| 61 | cgacgcgtcc catacgttgt tggcatttta attcttctct ctgcagcggc agcatgtgcc |
| 121 | gccgctgcag agagtttcta gcgatgacag cccctctggg caacgagccg ggggggctgt |
| 181 | c. |

In certain embodiments, the piggyBac or piggyBac-like transposon comprises at a sequence of:

| (SEQ ID NO: 14581) | |
|---|---|
| 1 | cctttttact gccaatgacg catgggatac gtcgtggcag taaaagggct taaatgccaa |
| 61 | cgacgcgtcc catacgttgt tggcatttta agtcttctct ctgcagcggc agcatgtgcc |
| 121 | gccgctgcag agagtttcta gcgatgacag cccctctggg caacgagccg ggggggctgt |
| 181 | c. |

In certain embodiments, the piggyBac or piggyBac-like transposon comprises at a sequence of:

| (SEQ ID NO: 14582) | |
|---|---|
| 1 | cctttttact gccaatgacg catgggatac gtcgtggcag taaaagggct taaatgccaa |
| 61 | cgacgcgtcc catacgttgt tggcatttta agtcttctct ctgcagcggc agcatgtgcc |
| 121 | gccgctgcag agag. |

In certain embodiments, the piggyBac or piggyBac-like transposon comprises at a sequence of:

| (SEQ ID NO: 14583) | |
|---|---|
| 1 | cctttttact gccaatgacg catgggatac gtcgtggcag taaaagggct taaatgccaa |
| 61 | cgacgcgtcc catacgttgt tggcatttta agtctt. |

In certain embodiments, the piggyBac or piggyBac-like transposon comprises at a sequence of:

| (SEQ ID NO: 14584) | |
|---|---|
| 1 | ccctttgcct gccaatcacg catgggatac gtcgtggcag taaaagggct taaatgccaa |
| 61 | cgacgcgtcc catacgttgt tggcatttta agtctt . |

In certain embodiments, the piggyBac or piggyBac-like transposon comprises at a sequence of:

| (SEQ ID NO: 14585) | |
|---|---|
| 1 | ttatcctttt tactgccaat gacgcatggg atacgtcgtg gcagtaaaag ggcttaaatg |
| 61 | ccaacgacgc gtcccatacg ttgttggcat tttaagtctt ctctctgcag cggcagcatg |
| 121 | tgccgccgct gcagagagtt tctagcgatg acagcccctc tgggcaacga gccggggggg |
| 181 | ctgtc. |

In certain embodiments, the piggyBac or piggyBac-like transposon comprises at a sequence of:

| (SEQ ID NO: 14586) | |
|---|---|
| 1 | tttgcatttt tagacattta gaagcctata tcttgttaca gaattggaat tacacaaaaa |
| 61 | ttctaccata ttttgaaagc ttaggttgtt ctgaaaaaaa caatatattg ttttcctggg |
| 121 | taaactaaaa gtcccctcga ggaaaggccc ctaaagtgaa acagtgcaaa acgttcaaaa |
| 181 | actgtctggc aatacaagtt ccactttggg acaaatcggc tggcagtgaa aggg. |

In certain embodiments, the piggyBac or piggyBac-like transposon comprises a left transposon end sequence selected from SEQ ID NO: 14573 and SEQ ID NOs: 14579-14585. In certain embodiments, the left transposon end sequence is preceded by a left target sequence. In certain embodiments, the piggyBac or piggyBac-like transposon comprises at a sequence of:

| (SEQ ID NO: 14587) | |
|---|---|
| 1 | tttgcatttt tagacattta gaagcctata tcttgttaca gaattggaat tacacaaaaa |
| 61 | ttctaccata ttttgaaagc ttaggttgtt ctgaaaaaaa caatatattg ttttcctggg |
| 121 | taaactaaaa gtcccctcga ggaaaggccc ctaaagtgaa acagtgcaaa acgttcaaaa |
| 181 | actgtctggc aatacaagtt ccactttgac caaaacggct ggcagtaaaa ggg. |

In certain embodiments, the piggyBac or piggyBac-like transposon comprises at a sequence of:

| (SEQ ID NO: 14588) | |
|---|---|
| 1 | ttgttctgaa aaaaacaata tattgttttc ctgggtaaac taaaagtccc ctcgaggaaa |
| 61 | ggcccctaaa gtgaaacagt gcaaaacgtt caaaaactgt ctggcaatac aagttccact |
| 121 | ttgaccaaaa cggctggcag taaaaggg. |

In certain embodiments, the piggyBac or piggyBac-like transposon comprises at a sequence of:

| (SEQ ID NO: 14589) | |
|---|---|
| 1 | tttgcatttt tagacattta gaagcctata tcttgttaca gaattggaat tacacaaaaa |
| 61 | ttctaccata ttttgaaagc ttaggttgtt ctgaaaaaaa caatatattg ttttcctggg |
| 121 | taaactaaaa gtcccctcga ggaaaggccc ctaaagtgaa acagtgcaaa acgttcaaaa |
| 181 | actgtctggc aatacaagtt ccactttgac caaaacggct ggcagtaaaa gggttat. |

In certain embodiments, the piggyBac or piggyBac-like transposon comprises at a sequence of:

| (SEQ ID NO: 14590) | |
|---|---|
| 1 | ttgttctgaa aaaaacaata tattgttttc ctgggtaaac taaaagtccc ctcgaggaaa |
| 61 | ggcccctaaa gtgaaacagt gcaaaacgtt caaaaactgt ctggcaatac aagttccact |
| 121 | ttgggacaaa tcggctggca gtgaaaggg. |

In certain embodiments, the piggyBac or piggyBac-like transposon comprises a right transposon end sequence selected from SEQ ID NO: 14574 and SEQ ID NOs: 14587-14590. In certain embodiments, the right transposon end sequence is followed by a right target sequence. In certain embodiments, the left and right transposon ends share a 14 repeated sequence inverted in orientation in the two ends (SEQ ID NO: 14575) adjacent to the target sequence. In certain embodiments, the piggyBac or piggyBac-like transposon comprises a left transposon end comprising a target sequence and a sequence that is selected from SEQ ID NOs: 14582-14584 and 14573, and a right transposon end comprising a sequence selected from SEQ ID NOs: 14588-14590 and 14574 followed by a right target sequence.

In certain embodiments, the left transposon end of the piggyBac or piggyBac-like transposon comprises

| | |
|---|---|
| 1 | atcacgcatg ggatacgtcg tggcagtaaa agggcttaaa tgccaacgac gcgtcccata |
| 61 | cgtt |

(SEQ ID NO: 14591), and an ITR. In certain embodiments, the left transposon end comprises

| | |
|---|---|
| 1 | atgacgcatg ggatacgtcg tggcagtaaa agggcttaaa tgccaacgac gcgtcccata |
| 61 | cgttgttggc attttaagtc tt |

(SEQ ID NO: 14592) and an ITR. In certain embodiments, the right transposon end of the piggyBac or piggyBac-like transposon comprises

| | |
|---|---|
| 1 | cctgggtaaa ctaaaagtcc cctcgaggaa aggcccctaa agtgaaacag tgcaaaacgt |
| 61 | tcaaaaactg tctggcaata caagttccac tttgggacaa atcggc |

(SEQ ID NO: 14593) and an ITR. In certain embodiments, the right transposon end comprises

| | |
|---|---|
| 1 | ttgttctgaa aaaaacaata tattgttttc ctgggtaaac taaaagtccc ctcgaggaaa |
| 61 | ggcccctaaa gtgaaacagt gcaaaacgtt caaaaactgt ctggcaatac aagttccact |
| 121 | ttgaccaaaa cggc |

### (SEQ ID NO: 14594) and an ITR.

In certain embodiments, one transposon end comprises a sequence that is at least 90%, at least 95%, at least 99% or any percentage in between identical to SEQ ID NO: 14573 and the other transposon end comprises a sequence that is at least 90%, at least 95%, at least 99% or any percentage in between identical to SEQ ID NO: 14574. In certain embodiments, one transposon end comprises at least 14, at least 16, at least 18, at least 20 or at least 25 contiguous nucleotides from SEQ ID NO: 14573 and one transposon end comprises at least 14, at least 16, at least 18, at least 20 or at least 25 contiguous nucleotides from SEQ ID NO: 14574. In certain embodiments, one transposon end comprises at least 14, at least 16, at least 18, at least 20 from SEQ ID NO: 14591, and the other end comprises at least 14, at least 16, at least 18, at least 20 from SEQ ID NO: 14593. In certain embodiments, each transposon end comprises SEQ ID NO: 14575 in inverted orientations.

In certain embodiments, the piggyBac or piggyBac-like transposon comprises a sequence selected from of SEQ ID NO: 14573, SEQ ID NO: 14579, SEQ ID NO: 14581, SEQ ID NO: 14582, SEQ ID NO: 14583, and SEQ ID NO: 14588, and a sequence selected from SEQ ID NO: 14587, SEQ ID NO: 14588, SEQ ID NO: 14589 and SEQ ID NO: 14586 and the piggyBac or piggyBac-like transposase comprises SEQ ID NO: 14517 or SEQ ID NO: 14518.

In certain embodiments, the piggyBac or piggyBac-like transposon comprises ITRs of CCCTTTGCCTGCCA (SEQ ID NO: 14622) (left ITR) and TGGCAGTGAAAGGG (SEQ ID NO: 14623) (right ITR) adjacent to the target sequences.

In certain embodiments of the methods of the disclosure, the transposase enzyme is a piggyBac or piggyBac-like transposase enzyme. In certain embodiments, the piggyBac or piggyBac-like transposase enzyme is isolated or derived from Helicoverpa armigera. The piggyBac or piggyBac-like transposase enzyme may comprise or consist of an amino acid sequence at least 5%, 10%, 15%, 20%, 25%, 30%, 35%, 40%, 45%, 50%, 55%, 60%, 65%, 70%, 75%, 80%, 85%, 90%, 95%, 99% or any percentage in between identical to:

| (SEQ ID NO: 14525) | |
|---|---|
| 1 | MASRQRLNHD EIATILENDD DYSPLDSESE KEDCVVEDDV WSDNEDAIVD FVEDTSAQED |
| 61 | PDNNIASRES PNLEVTSLTS HRIITLPQRS IRGKNNHVWS TTKGRTTGRT SAINIIRTNR |
| 121 | GPTRMCRNIV DPLLCFQLFI TDEIIHEIVK WTNVEIIVKR QNLKDISASY RDINTMEIWA |
| 181 | LVGILTLTAV MKDNHLSTDE LFDATFSGTR YVSVMSRERF EFLIRCIRMD DKTLRPTLRS |
| 241 | DDAFLPVRKI WEIFINQCRQ NHVPGSNLTV DEQLLGFRGR CPFRMYIPNK PDKYGIKFPM |
| 301 | MCAAATKYMI DAIPYLGKST KTNGLPLGEF YVKDLTKTVH GTNRNITCDN WFTSIPLAKN |
| 361 | MLQAPYNLTI VGTIRSNKRE MPEEIKNSRS RPVGSSMFCF DGPLTLVSYK PKPSKMVFLL |
| 421 | SSCDENAVIN ESNGKPDMIL FYNQTKGGVD SFDQMCKSMS ANRKTNRWPM AVFYGMLNMA |
| 481 | FVNSYIIYCH NKINKQEKPI SRKEFMKKLS IQLTTPWMQE RLQAPTLKRT LRDNITNVLK |
| 541 | NVVPASSENI SNEPEPKKRR YCGVCSYKKR RMTKAQCCKC KKAICGEHNI DVCQDCI. |

In certain embodiments, the piggyBac or piggyBac-like transposon is isolated or derived from Helicoverpa armigera. In certain embodiments, the piggyBac or piggyBac-like transposon comprises a sequence of:

| (SEQ ID NO: 14570) | |
|---|---|
| 1 | ttaaccctag aagcccaatc tacgtaaatt tgacgtatac cgcggcgaaa tatctctgtc |
| 61 | tctttcatgt ttaccgtcgg atcgccgcta acttctgaac caactcagta gccattggga |
| 121 | cctcgcagga cacagttgcg tcatctcggt aagtgccgcc attttgttgt actctctatt |
| 181 | acaacacacg tcacgtcacg tcgttgcacg tcattttgac gtataattgg gctttgtgta |
| 241 | acttttgaat ttgtttcaaa ttttttatgt ttgtgattta tttgagttaa tcgtattgtt |
| 301 | tcgttacatt tttcatataa taataatatt ttcaggttga gtacaaa. |

14570). In certain embodiments, the piggyBac or piggyBac-like transposon comprises a sequence of:

| (SEQ ID NO: 14528) | |
|---|---|
| 1 | agactgtttt tttctaagag acttctaaaa tattattacg agttgattta attttatgaa |
| 61 | aacatttaaa actagttgat tttttttata attacataat tttaagaaaa agtgttagag |
| 121 | gcttgatttt tttgttgatt ttttctaaga tttgattaaa gtgccataat agtattaata |
| 181 | aagagtattt tttaacttaa aatgtatttt atttattaat taaaacttca attatgataa |
| 241 | ctcatgcaaa aatatagttc attaacagaa aaaaatagga aaactttgaa gttttgtttt |
| 301 | tacacgtcat ttttacgtat gattgggctt tatagctagt taaatatgat tgggcttcta |
| 361 | gggttaa . |

in certain embodiments of the methods of the disclosure, the transposase enzyme is a piggyBac or piggyBac-like transposase enzyme. In certain embodiments, the piggyBac or piggyBac-like transposase enzyme is isolated or derived from Pectinophora gossypiella. The piggyBac or piggyBac-like transposase enzyme may comprise or consist of an amino acid sequence at least 5%, 10%, 15%, 20%, 25%, 30%, 35%, 40%, 45%, 50%, 55%, 60%, 65%, 70%, 75%, 80%, 85%, 90%, 95%, 99% or any percentage in between identical to:

| (SEQ ID NO: 14530) | |
|---|---|
| 1 | MDLRKQDEKI RQWLEQDIEE DSKGESDNSS SETEDIVEME VHKNTSSESE VSSESDYEPV |
| 61 | CPSKRQRTQI IESEESDNSE SIRPSRRQTS RVIDSDETDE DVMSSTPQNI PRNPNVIQPS |
| 121 | SRFLYGKNKH KWSSAAKPSS VRTSRRNIIH FIPGPKERAR EVSEPIDIFS LFISEDMLQQ |
| 181 | VVTFTNAEML IRKNKYKTET FTVSPTNLEE IRALLGLLFN AAAMKSNHLP TRMLFNTHRS |
| 241 | GTIFKACMSA ERLNFLIKCL RFDDKLTRNV RQRDDRFAPI RDLWQALISN FQKWYTPGSY |
| 301 | ITVDEQLVGF RGRCSFRMYI PNKPNKYGIK LVMAADVNSK YIVNAIPYLG KGTDPQNQPL |
| 361 | ATFFIKEITS TLHGTNRNIT MDNWFTSVPL ANELLMAPYN LTLVGTLRSN KREIPEKLKN |
| 421 | SKSRAIGTSM FCYDGDKTLV SYKAKSNKVV FILSTIHDQP DINQETGKPE MIHFYNSTKG |
| 481 | AVDTVDQMCS SISTNRKTQR WPLCVFYNML NLSIINAYVV YVYNNVRNNK KPMSRRDFVI |
| 541 | KLGDQLMEPW LRQRLQTVTL RRDIKVMIQD ILGESSDLEA PVPSVSNVRK IYYLCPSKAR |
| 601 | RMTKHRCIKC KQAICGPHNI DICSRCIE. |

In certain embodiments, the piggyBac or piggyBac-like transposon is isolated or derived from Pectinophora gossypiella. In certain embodiments, the piggyBac or piggyBac-like transposon comprises a sequence of:

| (SEQ ID NO: 14532) | |
|---|---|
| 1 | ttaaccctag ataactaaac attcgtccgc tcgacgacgc gctatgccgc gaaattgaag |
| 61 | tttacctatt attccgcgtc ccccgccccc gccgcttttt ctagcttcct gatttgcaaa |
| 121 | atagtgcatc gcgtgacacg ctcgaggtca cacgacaatt aggtcgaaag ttacaggaat |
| 181 | ttcgtcgtcc gctcgacgaa agtttagtaa ttacgtaagt ttggcaaagg taagtgaatg |
| 241 | aagtattttt ttataattat tttttaattc tttatagtga taacgtaagg tttatttaaa |
| 301 | tttattactt ttatagttat ttagccaatt gttataaatt ccttgttatt gctgaaaaat |
| 361 | ttgcctgttt tagtcaaaat ttattaactt ttcgatcgtt ttttag. |

In certain embodiments, the piggyBac or piggyBac-like transposon comprises a sequence of:

| (SEQ ID NO: 14571) | |
|---|---|
| 1 | tttcactaag taattttgtt cctatttagt agataagtaa cacataatta ttgtgatatt |
| 61 | caaaacttaa gaggtttaat aaataataat aaaaaaaaaa tggtttttat ttcgtagtct |
| 121 | gctcgacgaa tgtttagtta ttacgtaacc gtgaatatag tttagtagtc tagggttaa. |

In certain embodiments of the methods of the disclosure, the transposase enzyme is a piggyBac or piggyBac-like transposase enzyme. In certain embodiments, the piggyBac or piggyBac-like transposase enzyme is isolated or derived from Ctenoplusia agnata. The piggyBac or piggyBac-like transposase enzyme may comprise or consist of an amino acid sequence at least 5%, 10%, 15%, 20%, 25%, 30%, 35%, 40%, 45%, 50%, 55%, 60%, 65%, 70%, 75%, 80%, 85%, 90%, 95%, 99% or any percentage in between identical to:

| (SEQ ID NO: 14534) | |
|---|---|
| 1 | MASRQHLYQD EIAAILENED DYSPHDTDSE MEDCVTQDDV RSDVEDEMVD NIGNGTSPAS |
| 61 | RHEDPETPDP SSEASNLEVT LSSHRIIILP QRSIREKNNH IWSTTKGQSS GRTAAINIVR |
| 121 | TNRGPTRMCR NIVDPLLCFQ LFIKEEIVEE IVKWTNVEMV QKRVNLKDIS ASYRDTNEME |
| 181 | IWAIISMLTL SAVMKDNHLS TDELFNVSYG TRYVSVMSRE RFEFLLRLLR MGDKLLRPNL |
| 241 | RQEDAFTPVR KIWEIFINQC RLNYVPGTNL TVDEQLLGFR GRCPFRMYIP NKPDKYGIKF |
| 301 | PMVCDAATKY MVDAIPYLGK STKTQGLPLG EFYVKELTQT VHGTNRNVTC DNWFTSVPLA |
| 361 | KSLLNSPYNL TLVGTIRSNK REIPEEVKNS RSRQVGSSMF CFDGPLTLVS YKPKPSKMVF |
| 421 | LLSSCNEDAV VNQSNGKPDM ILFYNQTKGG VDSFDQMCSS MSTNRKTNRW PMAVFYGMLN |
| 481 | MAFVNSYIIY CHNMLAKKEK PLSRKDFMKK LSTDLTTPSM QKRLEAPTLK RSLRDNITNV |
| 541 | LKIVPQAAID TSFDEPEPKK RRYCGFCSYK KKRMTKTQCF KCKKPVCGEH NIDVCQDCI. |

In certain embodiments, the piggyBac or piggyBac-like transposon is isolated or derived from Ctenoplusia agnata. In certain embodiments, the piggyBac or piggyBac-like transposon comprises a sequence of:

| (SEQ ID NO: 14535) | |
|---|---|
| 1 | ttaaccctag aagcccaatc tacgtcattc tgacgtgtat gtcgccgaaa atactctgtc |
| 61 | tctttctcct gcacgatcgg attgccgcga acgctcgatt caacccagtt ggcgccgaga |
| 121 | tctattggag gactgcggcg ttgattcggt aagtcccgcc attttgtcat agtaacagta |
| 181 | ttgcacgtca gcttgacgta tatttgggct ttgtgttatt tttgtaaatt ttcaacgtta |
| 241 | gtttattatt gcatcttttt gttacattac tggtttattt gcatgtatta ctcaaatatt |
| 301 | atttttattt tagcgtagaa aataca. |

In certain embodiments, the piggyBac or piggyBac-like transposon comprises a sequence of:

```
(SEQ ID NO: 14536)
    1 agactgtttt ttttgtattt gcattatata
        ttatattcta aagttgattt aattctaaga
  61 aaaacattaa aataagtttc tttttgtaaa
        atttaattaa ttataagaaa aagtttaagt
121 tgatctcatt ttttataaaa atttgcaatg
        tttccaaagt tattattgta aaagaataaa
181 taaaagtaaa ctgagtttta attgatgttt
        tattatatca ttatactata tattacttaa
241 ataaaacaat aactgaatgt atttctaaaa
        ggaatcacta gaaaatatag tgatcaaaaa
301 tttacacgtc atttttgcgt atgattgggc
        tttataggtt ctaaaaatat gattgggcct
361 ctagggttaa.
```

In certain embodiments, the piggyBac or piggyBac-like transposon comprises an ITR sequence of CCCTAGAAGCCCAATC (SEQ ID NO: 14564).

In certain embodiments of the methods of the disclosure, the transposase enzyme is a piggyBac or piggyBac-like transposase enzyme. In certain embodiments, the piggyBac or piggyBac-like transposase enzyme is isolated or derived from Agrotis ipsilon. The piggyBac (PB) or piggyBac-like transposase enzyme may comprise or consist of an amino acid sequence at least 5%, 10%, 15%, 20%, 25%, 30%, 35%, 40%, 45%, 50%, 55%, 60%, 65%, 70%, 75%, 80%, 85%, 90%, 95%, 99% or any percentage in between identical to:

```
(SEQ ID NO: 14537)
    1 MESRQRLNQD EIATILENDD DYSPLDSDSE
        AEDRVVEDDV WSDNEDAMID YVEDTSRQED
  61 PDNNIASQES ANLEVTSLTS HRIISLPQRS
        ICGKNNHVWS TTKGRTTGRT SAINIIRTNR
121 GPTRMCRNIV DPLLCFQLFI TDEIIHEIVK
        WTNVEMIVKR QNLIDISASY RDTNTMEMWA
181 LVGILTLTAV MKDNHLSTDE LFDATFSGTR
        YVSVMSRERF EFLIRCMRMD DKTLRPTLRS
241 DDAFIPVRKL WEIFINQCRL NYVPGGNLTV
        DEQLLGFRGR CPFRMYIPNK PDKYGIRFPM
301 MCDAATKYMI DAIPYLGKST KTNGLPLGEF
        YVKELTKTVH GTNRNVTCDN WFTSIPLAKN
361 MLQAPYNLTI VGTIRSNKRE IPEEIKNSRS
        RPVGSSMFCF DGPLTLVSYK PKPSRMVFLL
421 SSCDENAVIN ESNGKPDMIL FYNQTKGGVD
        SFDQMCKSMS ANRKTNRWPM AVFYGMLNMA
481 FVNSYIIYCH NKINKQKKPI NRKEFMKNLS
        TDLTTPWMQE RLKAPTLKRT LRDNITNVLK
541 NVVPPSPANN SEEPGPKKRS YCGFCSYKKR
        RMTKTQFYKC KKAICGEHNI DVCQDCV.
```

In certain embodiments, the piggyBac or piggyBac-like transposon is isolated or derived from Agrotis ipsilon. In certain embodiments, the piggyBac or piggyBac-like transposon comprises a sequence of:

```
(SEQ ID NO: 14538)
    1 ttaaccctag aagcccaatc tacgtaaatt
        tgacgtatac cgcggcgaaa tatatctgtc
  61 tctttcacgt ttaccgtcgg attcccgcta
        acttcggaac caactcagta gccattgaga
121 actcccagga cacagttgcg tcatctcggt
        aagtgccgcc attttgttgt aatagacagg
181 ttgcacgtca ttttgacgta taattgggct
        ttgtgtaact tttgaaatta tttataattt
241 ttattgatgt gatttatttg agttaatcgt
        attgtttcgt tacatttttc atatgatatt
301 aatattttca gattgaatat aaa.
```

In certain embodiments, the piggyBac or piggyBac-like transposon comprises a sequence of:

```
(SEQ ID NO: 14539)
    1 agactgtttt ttttaaaagg cttataaagt
        attactattg cgtgatttaa ttttataaaa
  61 atatttaaaa ccagttgatt tttttaataa
        ttacctaatt ttaagaaaaa atgttagaag
121 cttgatattt ttgttgattt ttttctaaga
        tttgattaaa aggccataat tgtattaata
181 aagagtattt ttaacttcaa atttatttta
        tttattaatt aaaacttcaa ttatgataat
241 acatgcaaaa atatagttca tcaacagaaa
        aatataggaa aactctaata gttttatttt
301 tacacgtcat ttttacgtat gattgggctt
        tatagctagt caaatatgat tgggcttcta
361 gggttaa.
```

In certain embodiments of the methods of the disclosure, the transposase enzyme is a piggyBac or piggyBac-like transposase enzyme. In certain embodiments, the piggyBac or piggyBac-like transposase enzyme is isolated or derived from Megachile rotundata. The piggyBac (PB) or piggyBac-like transposase enzyme may comprise or consist of an amino acid sequence at least 5%, 10%, 15%, 20%, 25%, 30%, 35%, 40%, 45%, 50%, 55%, 60%, 65%, 70%, 75%, 80%, 85%, 90%, 95%, 99% or any percentage in between identical to:

```
(SEQ ID NO: 14540)
    1 MNGKDSLGEF YLDDLSDCLD CRSASSTDDE
        SDSSNIAIRK RCPIPLIYSD SEDEDMNNNV
  61 EDNNHFVKES NRYHYQIVEK YKITSKTKKW
        KDVTVTEMKK FLGLIILMGQ VKKDVLYDYW
121 STDPSIETPF FSKVMSRNRF LQIMQSWHFY
        NNNDISPNSH RLVKIQPVID YFKEKFNNVY
181 KSDQQLSLDE CLIPWRGRLS IKTYNPAKIT
        KYGILVRVLS EARTGYVSNF CVYAADGKKI
241 EETVLSVIGP YKNMWHHVYQ DNYYNSVNIA
        KIFLKNKLRV CGTIRKNRSL PQILQTVKLS
301 RGQHQFLRNG HTLLEVWNNG KRNVNMISTI
        HSAQMAESRN RSRTSDCPIQ KPISIIDYNK
361 YMKGVDRADQ YLSYYSIFRK TKKWTKRVVM
        FFINCALFNS FKVYTTLNGQ KITYKNFLHK
421 AALSLIEDCG TEEQGTDLPN SEPTTTRTTS
        RVDHPGRLEN FGKHKLVNIV TSGQCKKPLR
481 QCRVCASKKK LSRTGFACKY CNVPLHKGDC
        FERYHSLKKY.
```

In certain embodiments, the piggyBac or piggyBac-like transposon is isolated or derived from Megachile rotundata. In certain embodiments, the piggyBac or piggyBac-like transposon comprises a sequence of:

```
(SEQ ID NO: 14541)
    1 ttaaataatg cccactctag atgaacttaa
        cactttaccg accggccgtc gattattcga
  61 cgtttgctcc ccagcgctta ccgaccggcc
        atcgattatt cgacgtttgc ttcccagcgc
121 ttaccgaccg gtcatcgact tttgatcttt
        ccgttagatt tggttaggtc agattgacaa
181 gtagcaagca tttcgcattc tttattcaaa
        taatcggtgc ttttttctaa gctttagccc
241 ttagaa.
```

In certain embodiments, the the piggyBac or piggyBac-like transposon comprises a sequence of:

```
(SEQ ID NO: 14542)
    1 acaacttctt ttttcaacaa atattgttat
        atggattatt tatttattta tttatttatg
  61 gtatatttta tgtttattta tttatggtta
        ttatggtata ttttatgtaa ataataaact
121 gaaaacgatt gtaatagatg aaataaatat
        tgttttaaca ctaatataat taaagtaaaa
181 gattttaata aatttcgtta ccctacaata
        acacgaagcg tacaatttta ccagagttta
241 ttaa.
```

In certain embodiments of the methods of the disclosure, the transposase enzyme is a piggyBac or piggyBac-like transposase enzyme. In certain embodiments, the piggyBac or piggyBac-like transposase enzyme is isolated or derived from Bombus impatiens. The piggyBac (PB) or piggyBac-like transposase enzyme may comprise or consist of an amino acid sequence at least 5%, 10%, 15%, 20%, 25%, 30%, 35%, 40%, 45%, 50%, 55%, 60%, 65%, 70%, 75%, 80%, 85%, 90%, 95%, 99% or any percentage in between identical to:

```
(SEQ ID NO: 14543)
    1 MNEKNGIGEF YLDDLSDCPD SYSRSNSGDE
        SDGSDTIIRK RGSVLPPRYS DSEDDEINNV
  61 EDNANNVENN DDIWSTNDEA IILEPFEGSP
        GLKIMPSSAE SVTDNVNLFF GDDFFEHLVR
121 ESNRYHYQVM EKYKIPSKAK KWTDITVPEM
        KKFLGLIVLM GQIKKDVLYD YWSTDPSIET
181 PFFSQVMSRN RFVQIMQSWH FCNNDNIPHD
        SHRLAKIQPV IDYFRRKFND VYKPCQQLSL
241 DESIIPWRGR LSIKTYNPAK ITKYGILVRV
        LSEAVTGYVC NFDVYAADGK KLEDTAVIEP
301 YKNIWHQIYQ DNYYNSVKMA RILLKNKVRV
        CGTIRKNRGL PRSLKTIQLS RGQYEFRRNH
361 QILLEVWNNG RRNVNMISTI HSAQLMESRS
        KSKRSDVPIQ KPNSIIDYNK YMKGVDRADQ
421 YLAYYSIFRK TKKWTKRVVM FFINCALFNS
        FRVYTILNGK NITYKNFLHK VAVSWIEDGE
481 TNCTEQDDNL PNSEPTRRAP RLDHPGRLSN
        YGKHKLINIV TSGRSLKPQR QCRVCAVQKK
541 RSRTCFVCKF CNVPLHKGDC FERYHTLKKY.
```

In certain embodiments, the piggyBac or piggyBac-like transposon is isolated or derived from Bombus impatiens. In certain embodiments, the piggyBac or piggyBac-like transposon comprises a sequence of:

```
(SEQ ID NO: 14544)
    1 ttaatttttt aacattttac cgaccgatag
        ccgattaatc gggtttttgc cgctgacgct
  61 taccgaccga taacctatta atcggctttt
        tgtcgtcgaa gcttaccaac ctatagccta
121 cctatagtta atcggttgcc atggcgataa
        acaatctttc tcattatatg agcagtaatt
181 tgttatttag tactaaggta ccttgctcag
        ttgcgtcagt tgcgttgctt tgtaagctcc
241 cacagtttta taccaattcg aaaaacttac
        cgttcgcg.
```

In certain embodiments, the piggyBac or piggyBac-like transposon comprises a sequence of:

```
(SEQ ID NO: 14545)
    1 actatttcac atttgaacta aaaaccgttg
        taatagataa aataaatata atttagtatt
  61 aatattatgg aaacaaaaga ttttattcaa
        tttaattatc ctatagtaac aaaaagcggc
121 caattttatc tgagcatacg aaaagcacag
        atactcccgc ccgacagtct aaaccgaaac
181 agagccggcg ccagggagaa tctgcgcctg
        agcagccggt cggacgtgcg tttgctgttg
241 aaccgctagt ggtcagtaaa ccagaaccag
        tcagtaagcc agtaactgat cagttaacta
301 gattgtatag ttcaaattga acttaatcta
        gtttttaagc gtttgaatgt tgtctaactt
361 cgttatatat tatattcttt ttaa.
```

In certain embodiments of the methods of the disclosure, the transposase enzyme is a piggyBac or piggyBac-like transposase enzyme. In certain embodiments, the piggyBac or piggyBac-like transposase enzyme is isolated or derived from Mamestra brassicae. The piggyBac (PB) or piggyBac-like transposase enzyme may comprise or consist of an amino acid sequence at least 5%, 10%, 15%, 20%, 25%, 30%, 35%, 40%, 45%, 50%, 55%, 60%, 65%, 70%, 75%, 80%, 85%, 90%, 95%, 99% or any percentage in between identical to:

```
(SEQ ID NO: 14546)
    1 MFSFVPNKEQ TRTVLIFCFH LKTTAAESHR
        PLVEAFGEQV PTVKTCERWF QRFKSGDFDV
  61 DDKEHGKPPK RYEDAELQAL LDEDDAQTQK
        QLAEQLEVSQ QAVSNRLREG GKIQKVGRWV
121 PHELNERQRE RRKNTCEILL SRYKRKSFLH
        RIVTGEEKWI FFVNPKRKKS YVDPGQPATS
181 TARPNRFGKK TRLCVWWDQS GVIYYELLKP
        GETVNTARYQ QQLINLNRAL QRKRPEYQKR
241 QHRVIFLHDN APSHTARAVR DTLETLNWEV
        LPHAAYSPDL APSDYHLFAS MGHALAEQRF
301 DSYESVEEWL DEWFAAKDDE FYWRGIHKLP
        ERWDNCVASD GKYFE.
```

In certain embodiments, the piggyBac or piggyBac-like transposon is isolated or derived from Mamestra brassicae. In certain embodiments, the piggyBac or piggyBac-like transposon comprises a sequence of:

```
(SEQ ID NO: 14547)
    1 ttattgggtt gcccaaaaag taattgcgga
        tttttcatat acctgtcttt taaacgtaca
  61 tagggatcga actcagtaaa actttgacct
        tgtgaaataa caaacttgac tgtccaacca
121 ccatagtttg gcgcgaattg agcgtcataa
        ttgttttgac tttttgcagt caac.
```

In certain embodiments, the piggyBac or piggyBac-like transposon comprises a sequence of:

```
(SEQ ID NO: 14548)
  1 atgatttttt ctttttaaac caattttaat
      tagttaattg atataaaaat ccgcaattac
61 tttttgggca acccaataa.
```

In certain embodiments of the methods of the disclosure, the transposase enzyme is a piggyBac or piggyBac-like transposase enzyme. In certain embodiments, the piggyBac or piggyBac-like transposase enzyme is isolated or derived from Mayetiola destructor. The piggyBac (PB) or piggyBac-like transposase enzyme may comprise or consist of an amino acid sequence at least 5%, 10%, 15%, 20%, 25%, 30%, 35%, 40%, 45%, 50%, 55%, 60%, 65%, 70%, 75%, 80%, 85%, 90%, 95%, 99% or any percentage in between identical to:

```
(SEQ ID NO: 14549)
    1 MENFENWRKR RHLREVLLGH FFAKKTAAES
        HRLLVEVYGE HALAKTQCFE WFQRFKSGDF
  61 DTEDKERPGQ PKKFEDEELE ALLDEDCCQT
        QEELAKSLGV TQQAISKRLK AAGYIQKQGN
121 WVPHELKPRD VERRFCMSEM LLQRHKKKSF
        LSRIITGDEK WIHYDNSKRK KSYVKRGGRA
181 KSTPKSNLHG AKVMLCIWWD QRGVLYYELL
        EPGQTITGDL YRTQLIRLKQ ALAEKRPEYA
241 KRHGAVIFHH DNARPHVALP VKNYLENSGW
        EVLPHPPYSP DLAPSDYHLF RSMQNDLAGK
301 RFTSEQGIRK WLDSFLAAKP AKFFEKGIHE
        LSERWEKVIA SDGQYFE.
```

In certain embodiments, the piggyBac or piggyBac-like transposon is isolated or derived from Mayetiola destructor. In certain embodiments, the piggyBac or piggyBac-like transposon comprises a sequence of:

```
(SEQ ID NO: 14550)
    1 taagacttcc aaaatttcca cccgaacttt
        accttccccg cgcattatgt ctctcttttc
  61 accctctgat ccctggtatt gttgtcgagc
        acgatttata ttgggtgtac aacttaaaaa
121 ccggaattgg acgctagatg tccacactaa
        cgaatagtgt aaaagcacaa atttcatata
181 tacgtcattt tgaaggtaca tttgacagct
        atcaaaatca gtcaataaaa ctattctatc
241 tgtgtgcatc atattttttt attaact.
```

In certain embodiments, the piggyBac or piggyBac-like transposon comprises a sequence of:

| (SEQ ID NO: 14551) | |
|---|---|
| 1 | tgcattcatt cattttgtta tcgaaataaa gcattaattt tcactaaaaa attccggttt |
| 61 | ttaagttgta cacccaatat catccttagt gacaattttc aaatggcttt cccattgagc |
| 121 | tgaaaccgtg gctctagtaa gaaaaacgcc caacccgtca tcatatgcct tttttttctc |
| 181 | aacatccg. |

In certain embodiments of the methods of the disclosure, the transposase enzyme is a piggyBac or piggyBac-like transposase enzyme. In certain embodiments, the piggyBac or piggyBac-like transposase enzyme is isolated or derived from Apis mellifera. The piggyBac (PB) or piggyBac-like transposase enzyme may comprise or consist of an amino acid sequence at least 5%, 10%, 15%, 20%, 25%, 30%, 35%, 40%, 45%, 50%, 55%, 60%, 65%, 70%, 75%, 80%, 85%, 90%, 95%, 99% or any percentage in between identical to:

| (SEQ ID NO: 14552) | |
|---|---|
| 1 | MENQKEHYRH ILLFYFRKGK NASQAHKKLC AVYGDEALKE RQCQNWFDKF RSGDFSLKDE |
| 61 | KRSGRPVEVD DDLIKAIIDS DRHSTTREIA EKLHVSHTCI ENHLKQLGYV QKLDTWVPHE |
| 121 | LKEKHLTQRI NSCDLLKKRN ENDPFLKRLI TGDEKWVVYN NIKRKRSWSR PREPAQTTSK |
| 181 | AGIHRKKVLL SVWWDYKGIV YFELLPPNRT INSVVYIEQL TKLNNAVEEK RPELTNRKGV |
| 241 | VFHHDNARPH TSLVTRQKLL ELGWDVLPHP PYSPDLAPSD YFLFRSLQNS LNGKNFNNDD |
| 301 | DIKSYLIQFF ANKNQKFYER GIMMLPERWQ KVIDQNGQHI TE. |

In certain embodiments, the piggyBac or piggyBac-like transposon is isolated or derived from Apis mellifera. In certain embodiments, the piggyBac or piggyBac-like transposon comprises a sequence of:

| (SEQ ID NO: 14553) | |
|---|---|
| 1 | ttgggttggc aactaagtaa ttgcggattt cactcataga tggcttcagt tgaattttta |
| 61 | ggtttgctgg cgtagtccaa atgtaaaaca cattttgtta tttgatagtt ggcaattcag |
| 121 | ctgtcaatca gtaaaaaaag ttttttgatc ggttgcgtag ttttcgtttg gcgttcgttg |
| 181 | aaaa. |

In certain embodiments, the piggyBac or piggyBac-like transposon comprises a sequence of:

| (SEQ ID NO: 14554) | |
|---|---|
| 1 | agttatttag ttccatgaaa aaattgtctt tgattttcta aaaaaaatcc gcaattactt |
| 61 | agttgccaat ccaa. |

In certain embodiments of the methods of the disclosure, the transposase enzyme is a piggyBac or piggyBac-like transposase enzyme. In certain embodiments, the piggyBac or piggyBac-like transposase enzyme is isolated or derived from Messor bouvieri. The piggyBac (PB) or piggyBac-like transposase enzyme may comprise or consist of an amino acid sequence at least 5%, 10%, 15%, 20%, 25%, 30%, 35%, 40%, 45%, 50%, 55%, 60%, 65%, 70%, 75%, 80%, 85%, 90%, 95%, 99% or any percentage in between identical to:

| (SEQ ID NO: 14555) | |
|---|---|
| 1 | MSSFVPENVH LRHALLFLFH QKKRAAESHR LLVETYGEHA PTIRTCETWF RQFKCGDFNV |
| 61 | QDKERPGRPK TFEDAELQEL LDEDSTQTQK QLAEKLNVSR VAICERLQAM GKIQKMGRWV |
| 121 | PHELNDRQME NRKIVSEMLL QRYERKSFLH RIVTGDEKWI YFENPKRKKS WLSPGEAGPS |
| 181 | TARPNRFGRK TMLCVWWDQI GVVYYELLKP GETVNTDRYR QQMINLNCAL IEKRPQYAQR |
| 241 | HDKVILQHDN APSHTAKPVK EMLKSLGWEV LSHPPYSPDL APSDYHLFAS MGHALAEQHF |
| 301 | ADFEEVKKWL DEWFSSKEKL FFWNGIHKLS ERWTKCIESN GQYFE. |

In certain embodiments, the piggyBac or piggyBac-like transposon is isolated or derived from Messor bouvieri. In certain embodiments, the piggyBac or piggyBac-like transposon comprises a sequence of:

| (SEQ ID NO: 14556) | |
|---|---|
| 1 | agtcagaaat gacacctcga tcgacgacta atcgacgtct aatcgacgtc gattttatgt |
| 61 | caacatgtta ccaggtgtgt cggtaattcc tttccggttt ttccggcaga tgtcactagc |
| 121 | cataagtatg aaatgttatg atttgataca tatgtcattt tattctactg acattaacct |
| 181 | taaaactaca caagttacgt tccgccaaaa taacagcgtt atagatttat aattttttga |
| 241 | aa. |

In certain embodiments, the piggyBac or piggyBac-like transposon comprises a sequence of:

| (SEQ ID NO: 14557) | |
|---|---|
| 1 | ataaatttga actatccatt ctaagtaacg tgttttcttt aacgaaaaaa ccggaaaaga |
| 61 | attaccgaca ctcctggtat gtcaacatgt tattttcgac attgaatcgc gtcgattcga |
| 121 | agtcgatcga ggtgtcattt ctgact. |

In certain embodiments of the methods of the disclosure, the transposase enzyme is a piggyBac or piggyBac-like transposase enzyme. In certain embodiments, the piggyBac or piggyBac-like transposase enzyme is isolated or derived from Trichoplusia ni. The piggyBac (PB) or piggyBac-like transposase enzyme may comprise or consist of an amino acid sequence at least 5%, 10%, 15%, 20%, 25%, 30%, 35%, 40%, 45%, 50%, 55%, 60%, 65%, 70%, 75%, 80%, 85%, 90%, 95%, 99% or any percentage in between identical to:

| (SEQ ID NO: 14558) | |
|---|---|
| 1 | MGSSLDDEHI LSALLQSDDE LVGEDSDSEV SDHVSEDDVQ SDTEEAFIDE VHEVQPTSSG |
| 61 | SEILDEQNVI EQPGSSLASN RILTLPQRTI RGKNKHCWST SKSTRRSRVS ALNIVRSQRG |
| 121 | PTRMCRNIYD PLLCFKLFFT DEIISEIVKW TNAEISLKRR ESMTSATFRD TNEDEIYAFF |
| 181 | GILVMTAVRK DNHMSTDDLF DRSLSMVYVS VMSRDRFDFL IRCLRMDDKS IRPTLRENDV |
| 241 | FTPVRKIWDL FIHQCIQNYT PGAHLTIDEQ LLGFRGRCPF RVYIPNKPSK YGIKILMMCD |
| 301 | SGTKYMINGM PYLGRGTQTN GVPLGEYYVK ELSKPVHGSC RNITCDNWFT SIPLAKNLLQ |
| 361 | EPYKLTIVGT VRSNKREIPE VLKNSRSRPV GTSMFCFDGP LTLVSYKPKP AKMVYLLSSC |
| 421 | DEDASINEST GKPQMVMYYN QTKGGVDTLD QMCSVMTCSR KTNRWPMALL YGMINIACIN |
| 481 | SFIIYSHNVS SKGEKVQSRK KFMRNLYMSL TSSFMRKRLE APTLKRYLRD NISNILPKEV |
| 541 | PGTSDDSTEE PVMKKRTYCT YCPSKIRRKA NASCKKCKKV ICREHNIDMC QSCF. |

In certain embodiments, the piggyBac or piggyBac-like transposon is isolated or derived from Trichoplusia ni. In certain embodiments, the piggyBac or piggyBac-like transposon comprises a sequence of:

| (SEQ ID NO: 14559) | |
|---|---|
| 1 | ttaaccctag aaagatagtc tgcgtaaaat tgacgcatgc attcttgaaa tattgctctc |
| 61 | tctttctaaa tagcgcgaat ccgtcgctgt gcatttagga catctcagtc gccgcttgga |
| 121 | gctcccgtga ggcgtgcttg tcaatgcggt aagtgtcact gattttgaac tataacgacc |
| 181 | gcgtgagtca aaatgacgca tgattatctt ttacgtgact tttaagattt aactcatacg |
| 241 | ataattatat tgttatttca tgttctactt acgtgataac ttattatata tatattttct |
| 301 | tgttatagat atc. |

In certain embodiments, the piggyBac or piggyBac-like transposon comprises a sequence of:

| (SEQ ID NO: 14560) | |
|---|---|
| 1 | tttgttactt tatagaagaa attttgagtt tttgtttttt tttaataaat aaataaacat |
| 61 | aaataaattg tttgttgaat ttattattag tatgtaagtg taaatataat aaaacttaat |
| 121 | atctattcaa attaataaat aaacctcgat atacagaccg ataaaacaca tgcgtcaatt |
| 181 | ttacgcatga ttatctttaa cgtacgtcac aatatgatta tctttctagg gttaa. |

In certain embodiments, the piggyBac or piggyBac-like transposon comprises a sequence of:

| (SEQ ID NO: 14561) | |
|---|---|
| 1 | ccctagaaag atagtctgcg taaaattgac gcatgcattc ttgaaatatt gctctctctt |
| 61 | tctaaatagc gcgaatccgt cgctgtgcat ttaggacatc tcagtcgccg cttggagctc |
| 121 | ccgtgaggcg tgcttgtcaa tgcggtaagt gtcactgatt ttgaactata acgaccgcgt |
| 181 | gagtcaaaat gacgcatgat tatcttttac gtgactttta agatttaact catacgataa |
| 241 | ttatattgtt atttcatgtt ctacttacgt gataacttat tatatatata ttttcttgtt |
| 301 | atagatatc. |

In certain embodiments, the piggyBac or piggyBac-like transposon comprises a sequence of:

| (SEQ ID NO: 14562) | |
|---|---|
| 1 | tttgttactt tatagaagaa attttgagtt tttgtttttt tttaataaat aaataaacat |
| 61 | aaataaattg tttgttgaat ttattattag tatgtaagtg taaatataat aaaacttaat |
| 121 | atctattcaa attaataaat aaacctcgat atacagaccg ataaaacaca tgcgtcaatt |
| 181 | ttacgcatga ttatctttaa cgtacgtcac aatatgatta tctttctagg g. |

In certain embodiments, the piggyBac or piggyBac-like transposon comprises a sequence of:

| (SEQ ID NO: 14609) | |
|---|---|
| 1 | tctaaatagc gcgaatccgt cgctgtgcat ttaggacatc tcagtcgccg cttggagctc |
| 61 | ccgtgaggcg tgcttgtcaa tgcggtaagt gtcactgatt ttgaactata acgaccgcgt |
| 121 | gagtcaaaat gacgcatgat tatcttttac gtgactttta agatttaact catacgataa |
| 181 | ttatattgtt atttcatgtt ctacttacgt gataacttat tatatatata ttttcttgtt |
| 241 | atagatatc. |

In certain embodiments, the piggyBac or piggyBac-like transposon comprises a sequence of:

| (SEQ ID NO: 14610) | |
|---|---|
| 1 | tttgttactt tatagaagaa attttgagtt tttgtttttt tttaataaat aaataaacat |
| 61 | aaataaattg tttgttgaat ttattattag tatgtaagtg taaatataat aaaacttaat |
| 121 | atctattcaa attaataaat aaacctcgat atacagaccg ataaaacaca tgcgtcaatt |
| 181 | ttacgcatga ttatctttaa cgtacgtcac aatatgatta tctttctagg g. |

In certain embodiments, the piggyBac or piggyBac-like transposon comprises SEQ ID NO: 14561 and SEQ ID NO: 14562, and the piggyBac or piggyBac-like transposase comprises SEQ ID NO: 14558. In certain embodiments, the piggyBac or piggyBac-like transposon comprises SEQ ID NO: 14609 and SEQ ID NO: 14610, and the piggyBac or piggyBac-like transposase comprises SEQ ID NO: 14558.

In certain embodiments, the piggyBac or piggyBac-like transposon is isolated or derived from Aphis gossypii. In certain embodiments, the piggyBac or piggyBac-like transposon comprises an ITR sequence of CCTTCCAGCGGGCGCGC (SEQ ID NO: 14565).

In certain embodiments, the piggyBac or piggyBac-like transposon is isolated or derived from Chilo suppressalis. In certain embodiments, the piggyBac or piggyBac-like transposon comprises an ITR sequence of CCCAGATTAGCCT (SEQ ID NO: 14566).

In certain embodiments, the piggyBac or piggyBac-like transposon is isolated or derived from Heliothis virescens. In certain embodiments, the piggyBac or piggyBac-like transposon comprises an ITR sequence of CCCTTAATTACTCGCG (SEQ ID NO: 14567).

In certain embodiments, the piggyBac or piggyBac-like transposon is isolated or derived from Pectinophora gossypiella. In certain embodiments, the piggyBac or piggyBac-like transposon comprises an ITR sequence of CCCTAGATAACTAAAC (SEQ ID NO: 14568).

In certain embodiments, the piggyBac or piggyBac-like transposon is isolated or derived from Anopheles stephensi. In certain embodiments, the piggyBac or piggyBac-like transposon comprises an ITR sequence of CCCTAGAAAGATA (SEQ ID NO: 14569).

### Gene Editing

In various embodiments, nucleases that may be used as cutting enzymes include, but are not limited to, Cas9, transcription activator-like effector nucleases (TALENs) and zinc finger nucleases. In certain embodiments, the Cas9 is a catalytically inactive or “inactivated” Cas9 (dCas9). In certain embodiments, the Cas9 is a catalytically inactive or “inactivated” nuclease domain of Cas9. In certain embodiments, the dCas9 is encoded by a shorter sequence that is derived from a full length, catalytically inactivated, Cas9, referred to herein as a “small” dCas9 or dSaCas9.

In certain embodiments, the inactivated, small, Cas9 (dSaCas9) operatively-linked to an active nuclease. In certain embodiments, the disclosure provides a fusion protein comprising, consisting essentially of or consisting of a DNA binding domain and molecule nuclease, wherein the nuclease comprises a small, inactivated Cas9 (dSaCas9). In certain embodiments, the dSaCas9 of the disclosure comprises the mutations D10A and N580A (underlined and bolded) which inactivate the catalytic site. In certain embodiments, the dSaCas9 (isolated or derived from Staphylococcus aureus) of the disclosure comprises the amino acid sequence of:

| (SEQ ID NO: 14497) | |
|---|---|
| 1 | MKRNYILGLA IGITSVGYGI IDYETRDVID AGVRLFKEAN VENNEGRRSK RGARRLKRRR |
| 61 | RHRIQRVKKL LFDYNLLTDH SELSGINPYE ARVKGLSQKL SEEEFSAALL HLAKRRGVHN |
| 121 | VNEVEEDTGN ELSTKEQISR NSKALEEKYV AELQLERLKK DGEVRGSINR FKTSDYVKEA |
| 181 | KQLLKVQKAY HQLDQSFIDT YIDLLETRRT YYEGPGEGSP FGWKDIKEWY EMLMGHCTYF |
| 241 | PEELRSVKYA YNADLYNALN DLNNLVITRD ENEKLEYYEK FQIIENVFKQ KKKPTLKQIA |
| 301 | KEILVNEEDI KGYRVTSTGK PEFTNLKVYH DIKDITARKE IIENAELLDQ IAKILTIYQS |
| 361 | SEDIQEELTN LNSELTQEEI EQISNLKGYT GTHNLSLKAI NLILDELWHT NDNQIAIFNR |
| 421 | LKLVPKKVDL SQQKEIPTTL VDDFILSPVV KRSFIQSIKV INAIIKKYGL PNDIIIELAR |
| 481 | EKNSKDAQKM INEMQKRNRQ TNERIEEIIR TTGKENAKYL IEKIKLHDMQ EGKCLYSLEA |
| 541 | IPLEDLLNNP FNYEVDHIIP RSVSFDNSFN NKVLVKQEEA SKKGNRTPFQ YLSSSDSKIS |
| 601 | YETFKKHILN LAKGKGRISK TKKEYLLEER DINRFSVQKD FINRNLVDTR YATRGLMNLL |
| 661 | RSYFRVNNLD VKVKSINGGF TSFLRRKWKF KKERNKGYKH HAEDALIIAN ADFIFKEWKK |
| 721 | LDKAKKVMEN QMFEEKQAES MPEIETEQEY KEIFITPHQI KHIKDFKDYK YSHRVDKKPN |
| 781 | RELINDTLYS TRKDDKGNTL IVNNLNGLYD KDNDKLKKLI NKSPEKLLMY HHDPQTYQKL |
| 841 | KLIMEQYGDE KNPLYKYYEE TGNYLTKYSK KDNGPVIKKI KYYGNKLNAH LDITDDYPNS |
| 901 | RNKVVKLSLK PYRFDVYLDN GVYKFVTVKN LDVIKKENYY EVNSKCYEEA KKLKKISNQA |
| 961 | EFIASFYNND LIKINGELYR VIGVNNDLLN RIEVNMIDIT YREYLENMND KRPPRIIKTI |
| 1021 | ASKTQSIKKY STDILGNLYE VKSKKHPQII KKG. |

In certain embodiments of the gene editing systems of the disclosure, the dCas9 of the disclosure comprises a dCas9 isolated or derived from Streptococcus pyogenes. In certain embodiments, the dCas9 comprises a dCas9 with substitutions at positions 10 and 840 of the amino acid sequence of the dCas9 which inactivate the catalytic site. In certain embodiments, these substitutions are D10A and H840A. In certain embodiments, the amino acid sequence of the dCas9 (isolated or derived from Streptococcus pyogenes) comprises the sequence of:

```
(SEQ ID NO: 14498)
      1 XDKKYSIGLA IGTNSVGWAV ITDEYKVPSK KFKVLGNTDR HSIKKNLIGA LLFDSGETAE
    61 ATRLKRTARR RYTRRKNRIC YLQEIFSNEM AKVDDSFFHR LEESFLVEED KKHERHPIFG
  121 NIVDEVAYHE KYPTIYHLRK KLVDSTDKAD LRLIYLALAH MIKFRGHFLI EGDLNPDNSD
  181 VDKLFIQLVQ TYNQLFEENP INASGVDAKA ILSARLSKSR RLENLIAQLP GEKKNGLFGN
  241 LIALSLGLTP NFKSNFDLAE DAKLQLSKDT YDDDLDNLLA QIGDQYADLF LAAKNLSDAI
  301 LLSDILRVNT EITKAPLSAS MIKRYDEHHQ DLTLLKALVR QQLPEKYKEI FFDQSKNGYA
  361 GYIDGGASQE EFYKFIKPIL EKMDGTEELL VKLNREDLLR KQRTFDNGSI PHQIHLGELH
  421 AILRRQEDFY PFLKDNREKI EKILTFRIPY YVGPLARGNS RFAWMTRKSE ETITPWNFEE
  481 VVDKGASAQS FIERMTNFDK NLPNEKVLPK HSLLYEYFTV YNELTKVKYV TEGMRKPAFL
  541 SGEQKKAIVD LLFKTNRKVT VKQLKEDYFK KIECFDSVEI SGVEDRFNAS LGTYHDLLKI
  601 IKDKDFLDNE ENEDILEDIV LTLTLFEDRE MIEERLKTYA HLFDDKVMKQ LKRRRYTGWG
  661 RLSRKLINGI RDKQSGKTIL DFLKSDGFAN RNFMQLIHDD SLTFKEDIQK AQVSGQGDSL
  721 HEHIANLAGS PAIKKGILQT VKVVDELVKV MGRHKPENIV IEMARENQTT QKGQKNSRER
  781 MKRIEEGIKE LGSQILKEHP VENTQLQNEK LYLYYLQNGR DMYVDQELDI NRLSDYDVDA
  841 IVPQSFLKDD SIDNKVLTRS DKNRGKSDNV PSEEVVKKMK NYWRQLLNAK LITQRKFDNL
  901 TKAERGGLSE LDKAGFIKRQ LVETRQITKH VAQILDSRMN TKYDENDKLI REVKVITLKS
  961 KLVSDFRKDF QFYKVREINN YHHAHDAYLN AVVGTALIKK YPKLESEFVY GDYKVYDVRK
1021 MIAKSEQEIG KATAKYFFYS NIMNFFKTEI TLANGEIRKR PLIETNGETG EIVWDKGRDF
1081 ATVRKVLSMP QVNIVKKTEV QTGGFSKESI LPKRNSDKLI ARKKDWDPKK YGGFDSPTVA
1141 YSVLVVAKVE KGKSKKLKSV KELLGITIME RSSFEKNPID FLEAKGYKEV KKDLIIKLPK
1201 YSLFELENGR KRMLASAGEL QKGNELALPS KYVNFLYLAS HYEKLKGSPE DNEQKQLFVE
1261 QHKHYLDEII EQISEFSKRV ILADANLDKV LSAYNKHRDK PIREQAENII HLFTLTNLGA
1321 PAAFKYFDTT IDRKRYTSTK EVLDATLIHQ SITGLYETRI DLSQLGGD.
```

In certain embodiments of the gene editing systems of the disclosure, the nuclease domain may comprise, consist essentially of or consist of a dCas9 or a dSaCas9 and a type IIS endonuclease. In certain embodiments of the disclosure, the nuclease domain may comprise, consist essentially of or consist of a dSaCas9 and a type IIS endonuclease, including, but not limited to, AciI, Mn1I, AlwI, BbvI, BccI, BceAI, BsmAI, BsmFI, BspCNI, BsrI, BtsCI, HgaI, HphI, HpyAV, MbolI, My1I, PleI, SfaNI, AcuI, BciVI, BfuAI, BmgBI, BmrI, BpmI, BpuEI, BsaI, BseRI, BsgI, BsmI, BspMI, BsrBI, BsrBI, BsrDI, BtgZI, BtsI, EarI, EciI, MmeI, NmeAIII, BbvCI, Bpu10I, BspQI, SapI, BaeI, BsaXI, CspCI, BfiI, MboII, Acc36I, FokI or Clo051. In certain embodiments of the disclosure, the nuclease domain may comprise, consist essentially of or consist of a dSaCas9 and Clo051. An exemplary Clo051 nuclease domain may comprise, consist essentially of or consist of, the amino acid sequence of:

(SEQ ID NO: 14503)
EGIKSNISLLKDELRGQISHISHEYLSLIDLAFDSKQNRLFEMKVLELLV
NEYGFKGRHLGGSRKPDGIVYSTTLEDNFGIIVDTKAYSEGYSLPISQAD
EMERYVRENSNRDEEVNPNKWWENFSEEVKKYYFVFISGSFKGKFEEQLR
RLSMTTGVNGSAVNVVNLLLGAEKIRSGEMTIEELERAMFNNSEFILKY.

An exemplary dCas9-Clo051 nuclease domain may comprise, consist essentially of or consist of, the amino acid sequence of (Clo051 sequence underlined, linker bold italics, dCas9 (Staphylococcus pyogenes) sequence in italics):

(SEQ ID NO: 14654)
MAPKKKRKVEGIKSNISLLKDELRGQISHISHEYLSLIDLAFDSKQNRLF
EMKVLELLVNEYGFKGRHLGGSRKPDGIVYSTTLEDNFGIIVDTKAYSEG
YSLPISQADEMERYVRENSNRDEEVNPNKWWENFSEEVKKYYFVFISGSF
KGKFEEQLRRLSMTTGVNGSAVNVVNLLLGAEKIRSGEMTIEELERAMFN
NSEFILKY [symbol] DKKYSIGLAIGTNSVGWAVITDEYKVPSKKFKVLGNT
DRHSIKKNLIGALLFDSGETAEATRLKRTARRRYTRRKNRICYLQEIFSN
EIVIAKVDDSFFHRLEESFLVEEDKKHERHPIFGNIVDEVAYHEKYPTIY
HLRKKLVDSTDKADLRLIYLALAHMIKFRGHFLIEGDLNPDNSDVDKLFI
QLVQTYNQLFEENPINASGVDAICAILSARLSKSRRLENLIAQLPGEKKN
GLFGNLIALSLGLTPNFKSNFDLAEDAKLQLSKDTYDDDLDNLLAQIGDQ
YADLFLAAKNLSDAILLSDILRVNTEITKAPLSASMIKRYDEHHQDLTLL
KALVRQQLPEKYKEIFFDQSKNGYAGYIDGGASQEEFYKFIKPILEICMD
GTEELLVKLNREDLLRKQRTFDNGSIPHQIHLGELHAILRRQEDFYPFLK
DNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGM
RKPAFLSGEQKKAIVDLLFKTNRKVTVKQLKEDYFKKIECFDSVEISGVE
DRFNASLGTYHDLLKIIKDKDFLDNEENEDILEDIVLTLTLFEDREAKEE
RLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIK
KGILQTVKVVDELVKVMGRHKPENIVIEIVIARENQTTQKGQKNSRERMI
CRIEEGIKELGSQILKEHPVENTQLQNEKLYLYYLQNGRDMYVDQELDIN
RLSDYDVDAIVPQSFLKDDSIDNKVLTRSDKNRGKSDNVPSEEVVKKMKN
YWRQLLNAKLITQRKFDNLTKAERGGLSELDKAGFIKRQLVETRQITKHV
AQILDSRMNTKYDENDKLIREVKVITLKSKLVSDFRKDFQFYKVREINNY
HHAHDAYLNAVVGTALIKKYPKLESEFVYGDYKVYDVRICAKAKSEQEIG
KATAKYFFYSNIMNFFKTEITLANGEIRKRPLIETNGETGEIVWDKGRDF
ATVRKVLSMPQVNIVKKTEVQTGGFSKESILPKRNSDKLIARKKDWDPKK
YGGFDSPTVAYSVLVVAKVEKGKSKKLKSVKELLGITIMERSSFEKNPID
FLEAKGYKEVKKDLIIKLPKYSLFELENGRKRMLASAGELQKGNELALPS
KYVNFLYLASHYEKLKGSPEDNEQKQLFVEQHKHYLDEHEQISEFSKRVI
LADANLDKVLSAYNKHRDKPIREQAENIHILFTLTNLGAPAAFKYFDTTI
DRKRYTSTKEVLDATLIHQSITGLYETRIDLSQLGGDGSPKKKRKVSS.

Gene editing compositions of the disclosure may comprise a nuclease protein or a nuclease domain thereof. In certain embodiments, the gene editing composition comprises a sequence encoding a nuclease protein or a sequence encoding a nuclease domain thereof. In certain embodiments, the sequence encoding a nuclease protein or the sequence encoding a nuclease domain thereof comprises a DNA sequence, an RNA sequence, or a combination thereof. In certain embodiments, the nuclease or the nuclease domain thereof comprises one or more of a CRISPR/Cas protein, a Transcription Activator-Like Effector Nuclease (TALEN), a Zinc Finger Nuclease (ZFN), and an endonuclease. In certain embodiments, the nuclease or the nuclease domain thereof comprises one or more of a nuclease-inactivated Cas (dCas) protein, a Transcription Activator-Like Effector Nuclease (TALEN), a Zinc Finger Nuclease (ZFN), and an endonuclease. In certain embodiments, the nuclease or the nuclease domain thereof comprises a nuclease-inactivated Cas (dCas) protein and an endonuclease. In certain embodiments, the nuclease or the nuclease domain thereof comprises a nuclease-inactivated Cas9 (dCas9) protein and an endonuclease, wherein the endonuclease comprises a Clo051 nuclease or a nuclease domain thereof. In certain embodiments, the gene editing composition comprises a fusion protein. In certain embodiments, the fusion protein comprises a nuclease-inactivated Cas9 (dCas9) protein and a Clo051 nuclease or a Clo051 nuclease domain. In certain embodiments, the gene editing composition further comprises a guide sequence. In certain embodiments, the guide sequence comprises an RNA sequence.

In certain embodiments, the gene editing composition comprises a fusion protein. In certain embodiments, the fusion protein comprises a nuclease-inactivated Cas9 (dCas9) protein and a Clo051 nuclease or a Clo051 nuclease domain. In certain embodiments, the gene editing composition further comprises a guide sequence. In certain embodiments, the guide sequence comprises an RNA sequence. In certain embodiments, the fusion protein comprises or consists of the amino acid sequence:

(SEQ ID NO: 14654)
MAPKKKRKVEGIKSNISLLKDELRGQISHISHEYLSLIDLAFDSKQNRLF
EMKVLELLVNEYGFKGRHLGGSRKPDGIVYSTTLEDNEGIIVDTKAYSEG
YSLPISQADEMERYVRENSNRDEEVNPNKWWENFSEEVKKYYFVFISGSF
KGKFEEQLRRLSMTTGVNGSAVNVVNLLLGAEKIRSGEMTIEELERAMEN
NSEFILKYGGGGSDKKYSIGLAIGTNSVGWAVITDEYKVPSKKEKVLGNT
DRHSIKKNLIGALLEDSGETAEATRLKRTARRRYTRRKNRICYLQEIFSN
EMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQL
VQTYNQLFEENPINASGVDAKAILSARLSKSRRLENLIAQLPGEKKNGLF
GNLIALSLGLIPNEKSNEDLAEDAKLQLSKDTYDDDLDNLLAQIGDQYAD
LFLAAKNLSDAILLSDILRVNTEITKAPLSASMIKRYDEHHQDLTLLKAL
VRQQLPEKYKEIFFDQSKNGYAGYIDGGASQEEFYKFIKPILEKMDGTEE
LLVKLNREDLLRKQRTFDNGSIPHQIHLGELHAILRRQEDFYPFLKDNRE
KIEKILTERIPYYVGPLARGNSRFAWMTRKSEETITPWNFEEVVDKGASA
QSFIERMTNEDKNLPNEKVLPKHSLLYEYFTVYNELTKVKYVTEGMRKPA
FLSGEQKKAIVDLLEKTNRKVIVKQLKEDYFKKIECEDSVEISGVEDRFN
ASLGTYHDLLKIIKDKDFLDNEENEDILEDIVLTLTLFEDREMIEERLKT
YAHLFDDKVMKQLKRRRYTGWGRLSRKLINGIRDKQSGKTILDFLKSDGF
ANRNFMQLIHDDSLTFKEDIQKAQVSGQGDSLHEHIANLAGSPAIKKGIL
QTVKVVDELVKVMGRHKPENIVIEMARENQTTQKGQKNSRERMKRIEEGI
KELGSQILKEHPVENTQLQNEKLYLYYLQNGRDMYVDQELDINRLSDYDV
DAIVPQSFLKDDSIDNKVLTRSDKNRGKSDNVPSEEVVKKMKNYWRQLLN
AKLITQRKFDNLTKAERGGLSELDKAGFIKRQLVETRQITKHVAQILDSR
MNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFF
YSNIMNFEKTEITLANGEIRKRPLIETNGETGEIVWDKGRDFATVRKVLS
MPQVNIVKKTEVQTGGFSKESILPKRNSDKLIARKKDWDPKKYGGFDSPT
VAYSVLVVAKVEKGKSKKLKSVKELLGITIMERSSFEKNPIDFLEAKGYK
EVKKDLIIKLPKYSLFELENGRKRMLASAGELQKGNELALPSKYVNFLYL
ASHYEKLKGSPEDNEQKQLFVEQHKHYLDEIIEQISEFSKRVILADANLD
KVLSAYNKHRDKPIREQAENIIHLFTLTNLGAPAAFKYFDTTIDRKRYTS
TKEVLDATLIHQSITGLYETRIDLSQLGGDGSPKKKRKVSS.

In certain embodiments, the fusion protein is encoded by a nucleic acid comprising or consisting of the sequence:

```
(SEQ ID NO: 14655)
      1 atggcaccaa agaagaaaag aaaagtggag ggcatcaagt caaacatcag cctgctgaaa
    61 gacgaactgc ggggacagat tagtcacatc agtcacgagt acctgtcact gattgatctg
  121 gccttcgaca gcaagcagaa tagactgttt gagatgaaag tgctggaact gctggtcaac
  181 gagtatggct tcaagggcag acatctgggc gggtctagga aacctgacgg catcgtgtac
  241 agtaccacac tggaagacaa cttcggaatc attgtcgata ccaaggctta ttccgagggc
  301 tactctctgc caattagtca ggcagatgag atggaaaggt acgtgcgcga aaactcaaat
  361 agggacgagg aagtcaaccc caataagtgg tgggagaatt tcagcgagga agtgaagaaa
  421 tactacttcg tctttatctc aggcagcttc aaagggaagt ttgaggaaca gctgcggaga
  481 ctgtccatga ctaccggggt gaacggatct gctgtcaacg tggtcaatct gctgctgggc
  541 gcagaaaaga tcaggtccgg ggagatgaca attgaggaac tggaacgcgc catgttcaac
  601 aattctgagt ttatcctgaa gtatggaggc gggggaagcg ataagaaata ctccatcgga
  661 ctggccattg gcaccaattc cgtgggctgg gctgtcatca cagacgagta caaggtgcca
  721 agcaagaagt tcaaggtcct ggggaacacc gatcgccaca gtatcaagaa aaatctgatt
  781 ggagccctgc tgttcgactc aggcgagact gctgaagcaa cccgactgaa gcggactgct
  841 aggcgccgat atacccggag aaaaaatcgg atctgctacc tgcaggaaat tttcagcaac
  901 gagatggcca aggtggacga tagtttcttt caccgcctgg aggaatcatt cctggtggag
  961 gaagataaga aacacgagcg gcatcccatc tttggcaaca ttgtggacga agtcgcttat
1021 cacgagaagt accctactat ctatcatctg aggaagaaac tggtggactc caccgataag
1081 gcagacctgc gcctgatcta tctggccctg gctcacatga tcaagttccg ggggcatttt
1141 ctgatcgagg gagatctgaa ccctgacaat tctgatgtgg acaagctgtt catccagctg
1201 gtccagacat acaatcagct gtttgaggaa aacccaatta atgcctcagg cgtggacgca
1261 aaggccatcc tgagcgccag actgtccaaa tctaggcgcc tggaaaacct gatcgctcag
1321 ctgccaggag agaagaaaaa cggcctgttt gggaatctga ttgcactgtc cctgggcctg
1381 acacccaact tcaagtctaa ttttgatctg gccgaggacg ctaagctgca gctgtccaaa
1441 gacacttatg acgatgacct ggataacctg ctggctcaga tcggcgatca gtacgcagac
1501 ctgttcctgg ccgctaagaa tctgagtgac gccatcctgc tgtcagatat tctgcgcgtg
1561 aacacagaga ttactaaggc cccactgagt gcttcaatga tcaaaagata tgacgagcac
1621 catcaggatc tgaccctgct gaaggctctg gtgaggcagc agctgcccga gaaatacaag
1681 gaaatcttct ttgatcagag caagaatgga tacgccggct atattgacgg cggggcttcc
1741 caggaggagt tctacaagtt catcaagccc attctggaaa agatggacgg caccgaggaa
1801 ctgctggtga agctgaatcg ggaggacctg ctgagaaaac agaggacatt tgataacgga
1861 agcatccctc accagattca tctgggcgaa ctgcacgcca tcctgcgacg gcaggaggac
1921 ttctacccat ttctgaagga taaccgcgag aaaatcgaaa agatcctgac cttcagaatc
1981 ccctactatg tggggcctct ggcacgggga aatagtagat ttgcctggat gacaagaaag
2041 tcagaggaaa ctatcacccc ctggaacttc gaggaagtgg tcgataaagg cgctagcgca
2101 cagtccttca ttgaaaggat gacaaatttt gacaagaacc tgccaaatga gaaggtgctg
2161 cccaaacaca gcctgctgta cgaatatttc acagtgtata acgagctgac taaagtgaag
2221 tacgtcaccg aagggatgcg caagcccgca ttcctgtccg gagagcagaa gaaagccatc
2281 gtggacctgc tgtttaagac aaatcggaaa gtgactgtca aacagctgaa ggaagactat
2341 ttcaagaaaa ttgagtgttt cgattcagtg gaaatcagcg gcgtcgagga caggtttaac
2401 gcctccctgg ggacctacca cgatctgctg aagatcatca aggataagga cttcctggac
2461 aacgaggaaa atgaggacat cctggaggac attgtgctga cactgactct gtttgaggat
2521 cgcgaaatga tcgaggaacg actgaagact tatgcccatc tgttcgatga caaagtgatg
2581 aagcagctga aaagaaggcg ctacaccgga tggggacgcc tgagccgaaa actgatcaat
2641 gggattagag acaagcagag cggaaaaact atcctggact ttctgaagtc cgatggcttc
2701 gccaacagga acttcatgca gctgattcac gatgactctc tgaccttcaa ggaggacatc
2761 cagaaagcac aggtgtctgg ccagggggac agtctgcacg agcatatcgc aaacctggcc
2821 ggcagccccg ccatcaagaa agggattctg cagaccgtga aggtggtgga cgaactggtc
2881 aaggtcatgg gacgacacaa acctgagaac atcgtgattg agatggcccg cgaaaatcag
2941 acaactcaga agggccagaa aaacagtcga gaacggatga agagaatcga ggaaggcatc
3001 aaggagctgg ggtcacagat cctgaaggag catcctgtgg aaaacactca gctgcagaat
3061 gagaaactgt atctgtacta tctgcagaat ggacgggata tgtacgtgga ccaggagctg
3121 gatattaaca gactgagtga ttatgacgtg gatgccatcg tccctcagag cttcctgaag
3181 gatgactcca ttgacaacaa ggtgctgacc aggtccgaca agaaccgcgg caaatcagat
3241 aatgtgccaa gcgaggaagt ggtcaagaaa atgaagaact actggaggca gctgctgaat
3301 gccaagctga tcacacagcg gaaatttgat aacctgacta aggcagaaag aggaggcctg
3361 tctgagctgg acaaggccgg cttcatcaag cggcagctgg tggagacaag acagatcact
3421 aagcacgtcg ctcagattct ggatagcaga atgaacacaa agtacgatga aaacgacaag
3481 ctgatcaggg aggtgaaagt cattactctg aaatccaagc tggtgtctga ctttagaaag
3541 gatttccagt tttataaagt cagggagatc aacaactacc accatgctca tgacgcatac
3601 ctgaacgcag tggtcgggac cgccctgatt aagaaatacc ccaagctgga gtccgagttc
3661 gtgtacggag actataaagt gtacgatgtc cggaagatga tcgccaaatc tgagcaggaa
3721 attggcaagg ccaccgctaa gtatttcttt tacagtaaca tcatgaattt ctttaagacc
3781 gaaatcacac tggcaaatgg ggagatcaga aaaaggcctc tgattgagac caacggggag
3841 acaggagaaa tcgtgtggga caagggaagg gattttgcta ccgtgcgcaa agtcctgtcc
3901 atgccccaag tgaatattgt caagaaaact gaagtgcaga ccgggggatt ctctaaggag
3961 agtattctgc ctaagcgaaa ctctgataaa ctgatcgccc ggaagaaaga ctgggacccc
4021 aagaagtatg gcgggttcga ctctccaaca gtggcttaca gtgtcctggt ggtcgcaaag
4081 gtggaaaagg ggaagtccaa gaaactgaag tctgtcaaag agctgctggg aatcactatt
4141 atggaacgca gctccttcga gaagaatcct atcgattttc tggaagccaa gggctataaa
4201 gaggtgaaga aagacctgat cattaagctg ccaaaatact cactgtttga gctggaaaac
4261 ggacgaaagc gaatgctggc aagcgccgga gaactgcaga agggcaatga gctggccctg
4321 ccctccaaat acgtgaactt cctgtatctg gctagccact acgagaaact gaaggggtcc
4381 cctgaggata acgaacagaa gcagctgttt gtggagcagc acaaacatta tctggacgag
4441 atcattgaac agatttcaga gttcagcaag agagtgatcc tggctgacgc aaatctggat
4501 aaagtcctga gcgcatacaa caagcaccga gacaaaccaa tccgggagca ggccgaaaat
4561 atcattcatc tgttcaccct gacaaacctg ggcgcccctg cagccttcaa gtattttgac
4621 accacaatcg atcggaagag atacacttct accaaagagg tgctggatgc taccctgatc
4681 caccagagta ttaccggcct gtatgagaca cgcatcgacc tgtcacagct gggaggcgat
4741 gggagcccca agaaaaagcg gaaggtgtct agttaa.
```

In certain embodiments, the gene editing composition comprises a fusion protein. In certain embodiments, the fusion protein comprises a nuclease-inactivated Cas9 (dCas9) protein and a Clo051 nuclease or a Clo051 nuclease domain. In certain embodiments, the gene editing composition further comprises a guide sequence. In certain embodiments, the guide sequence comprises an RNA sequence. In certain embodiments, the fusion protein comprises or consists of the amino acid sequence:

```
(SEQ ID NO: 14656)
      1 MPKKKRKVEG IKSNISLLKD ELRGQISHIS HEYLSLIDLA FDSKQNRLFE MKVLELLVNE
    61 YGFKGRHLGG SRKPDGIVYS TTLEDNFGII VDTKAYSEGY SLPISQADEM ERYVRENSNR
  121 DEEVNPNKWW ENFSEEVKKY YFVFISGSFK GKFEEQLRRL SMTTGVNGSA VNVVNLLLGA
  181 EKIRSGEMTI EELERAMFNN SEFILKYGGG GSDKKYSIGL AIGTNSVGWA VITDEYKVPS
  241 KKFKVLGNTD RHSIKKNLIG ALLFDSGETA EATRLKRTAR RRYTRRKNRI CYLQEIFSNE
  301 MAKVDDSFFH RLEESFLVEE DKKHERHPIF GNIVDEVAYH EKYPTIYHLR KKLVDSTDKA
  361 DLRLIYLALA HMIKFRGHFL IEGDLNPDNS DVDKLFIQLV QTYNQLFEEN PINASGVDAK
  421 AILSARLSKS RRLENLIAQL PGEKKNGLFG NLIALSLGLT PNFKSNFDLA EDAKLQLSKD
  481 TYDDDLDNLL AQIGDQYADL FLAAKNLSDA ILLSDILRVN TEITKAPLSA SMIKRYDEHH
  541 QDLTLLKALV RQQLPEKYKE IFFDQSKNGY AGYIDGGASQ EEFYKFIKPI LEKMDGTEEL
  601 LVKLNREDLL RKQRTFDNGS IPHQIHLGEL HAILRRQEDF YPFLKDNREK IEKILTFRIP
  661 YYVGPLARGN SRFAWMTRKS EETITPWNFE EVVDKGASAQ SFIERMTNFD KNLPNEKVLP
  721 KHSLLYEYFT VYNELTKVKY VTEGMRKPAF LSGEQKKAIV DLLFKTNRKV TVKQLKEDYF
  781 KKIECFDSVE ISGVEDRFNA SLGTYHDLLK IIKDKDFLDN EENEDILEDI VLTLTLFEDR
  841 EMIEERLKTY AHLFDDKVMK QLKRRRYTGW GRLSRKLING IRDKQSGKTI LDFLKSDGFA
  901 NRNFMQLIHD DSLTFKEDIQ KAQVSGQGDS LHEHIANLAG SPAIKKGILQ TVKVVDELVK
  961 VMGRHKPENI VIEMARENQT TQKGQKNSRE RMKRIEEGIK ELGSQILKEH PVENTQLQNE
1021 KLYLYYLQNG RDMYVDQELD INRLSDYDVD AIVPQSFLKD DSIDNKVLTR SDKNRGKSDN
1081 VPSEEVVKKM KNYWRQLLNA KLITQRKFDN LTKAERGGLS ELDKAGFIKR QLVETRQITK
1141 HVAQILDSRM NTKYDENDKL IREVKVITLK SKLVSDFRKD FQFYKVREIN NYHHAHDAYL
1201 NAVVGTALIK KYPKLESEFV YGDYKVYDVR KMIAKSEQEI GKATAKYFFY SNIMNFFKTE
1261 ITLANGEIRK RPLIETNGET GEIVWDKGRD FATVRKVLSM PQVNIVKKTE VQTGGFSKES
1321 ILPKRNSDKL IARKKDWDPK KYGGFDSPTV AYSVLVVAKV EKGKSKKLKS VKELLGITIM
1381 ERSSFEKNPI DFLEAKGYKE VKKDLIIKLP KYSLFELENG RKRMLASAGE LQKGNELALP
1441 SKYVNFLYLA SHYEKLKGSP EDNEQKQLFV EQHKHYLDEI IEQISEFSKR VILADANLDK
1501 VLSAYNKHRD KPIREQAENI IHLFTLINLG APAAFKYFDT TIDRKRYTST KEVLDATLIH
1561 QSITGLYETR IDLSQLGGDG SPKKKRKV.
```

In certain embodiments, the fusion protein is encoded by a nucleic acid comprising or consisting of the sequence:

```
(SEQ ID NO: 14657)
      1 atgcctaaga agaagcggaa ggtggaaggc atcaaaagca acatctccct cctgaaagac
    61 gaactccggg ggcagattag ccacattagt cacgaatacc tctccctcat cgacctggct
  121 ttcgatagca agcagaacag gctctttgag atgaaagtgc tggaactgct cgtcaatgag
  181 tacgggttca agggtcgaca cctcggcgga tctaggaaac cagacggcat cgtgtatagt
  241 accacactgg aagacaactt tgggatcatt gtggatacca aggcatactc tgagggttat
  301 agtctgccca tttcacaggc cgacgagatg gaacggtacg tgcgcgagaa ctcaaataga
  361 gatgaggaag tcaaccctaa caagtggtgg gagaacttct ctgaggaagt gaagaaatac
  421 tacttcgtct ttatcagcgg gtccttcaag ggtaaatttg aggaacagct caggagactg
  481 agcatgacta ccggcgtgaa tggcagcgcc gtcaacgtgg tcaatctgct cctgggcgct
  541 gaaaagattc ggagcggaga gatgaccatc gaagagctgg agagggcaat gtttaataat
  601 agcgagttta tcctgaaata cggtggcggt ggatccgata aaaagtattc tattggttta
  661 gccatcggca ctaattccgt tggatgggct gtcataaccg atgaatacaa agtaccttca
  721 aagaaattta aggtgttggg gaacacagac cgtcattcga ttaaaaagaa tcttatcggt
  781 gccctcctat tcgatagtgg cgaaacggca gaggcgactc gcctgaaacg aaccgctcgg
  841 agaaggtata cacgtcgcaa gaaccgaata tgttacttac aagaaatttt tagcaatgag
  901 atggccaaag ttgacgattc tttctttcac cgtttggaag agtccttcct tgtcgaagag
  961 gacaagaaac atgaacggca ccccatcttt ggaaacatag tagatgaggt ggcatatcat
1021 gaaaagtacc caacgattta tcacctcaga aaaaagctag ttgactcaac tgataaagcg
1081 gacctgaggt taatctactt ggctcttgcc catatgataa agttccgtgg gcactttctc
1141 attgagggtg atctaaatcc ggacaactcg gatgtcgaca aactgttcat ccagttagta
1201 caaacctata atcagttgtt tgaagagaac cctataaatg caagtggcgt ggatgcgaag
1261 gctattctta gcgcccgcct ctctaaatcc cgacggctag aaaacctgat cgcacaatta
1321 cccggagaga agaaaaatgg gttgttcggt aaccttatag cgctctcact aggcctgaca
1381 ccaaatttta agtcgaactt cgacttagct gaagatgcca aattgcagct tagtaaggac
1441 acgtacgatg acgatctcga caatctactg gcacaaattg gagatcagta tgcggactta
1501 tttttggctg ccaaaaacct tagcgatgca atcctcctat ctgacatact gagagttaat
1561 actgagatta ccaaggcgcc gttatccgct tcaatgatca aaaggtacga tgaacatcac
1621 caagacttga cacttctcaa ggccctagtc cgtcagcaac tgcctgagaa atataaggaa
1681 atattctttg atcagtcgaa aaacgggtac gcaggttata ttgacggcgg agcgagtcaa
1741 gaggaattct acaagtttat caaacccata ttagagaaga tggatgggac ggaagagttg
1801 cttgtaaaac tcaatcgcga agatctactg cgaaagcagc ggactttcga caacggtagc
1861 attccacatc aaatccactt aggcgaattg catgctatac ttagaaggca ggaggatttt
1921 tatccgttcc tcaaagacaa tcgtgaaaag attgagaaaa tcctaacctt tcgcatacct
1981 tactatgtgg gacccctggc ccgagggaac tctcggttcg catggatgac aagaaagtcc
2041 gaagaaacga ttactccatg gaattttgag gaagttgtcg ataaaggtgc gtcagctcaa
2101 tcgttcatcg agaggatgac caactttgac aagaatttac cgaacgaaaa agtattgcct
2161 aagcacagtt tactttacga gtatttcaca gtgtacaatg aactcacgaa agttaagtat
2221 gtcactgagg gcatgcgtaa acccgccttt ctaagcggag aacagaagaa agcaatagta
2281 gatctgttat tcaagaccaa ccgcaaagtg acagttaagc aattgaaaga ggactacttt
2341 aagaaaattg aatgcttcga ttctgtcgag atctccgggg tagaagatcg atttaatgcg
2401 tcacttggta cgtatcatga cctcctaaag ataattaaag ataaggactt cctggataac
2461 gaagagaatg aagatatctt agaagatata gtgttgactc ttaccctctt tgaagatcgg
2521 gaaatgattg aggaaagact aaaaacatac gctcacctgt tcgacgataa ggttatgaaa
2581 cagttaaaga ggcgtcgcta tacgggctgg ggacgattgt cgcggaaact tatcaacggg
2641 ataagagaca agcaaagtgg taaaactatt ctcgattttc taaagagcga cggcttcgcc
2701 aataggaact ttatgcagct gatccatgat gactctttaa ccttcaaaga ggatatacaa
2761 aaggcacagg tttccggaca aggggactca ttgcacgaac atattgcgaa tcttgctggt
2821 tcgccagcca tcaaaaaggg catactccag acagtcaaag tagtggatga gctagttaag
2881 gtcatgggac gtcacaaacc ggaaaacatt gtaatcgaga tggcacgcga aaatcaaacg
2941 actcagaagg ggcaaaaaaa cagtcgagag cggatgaaga gaatagaaga gggtattaaa
3001 gaactgggca gccagatctt aaaggagcat cctgtggaaa atacccaatt gcagaacgag
3061 aaactttacc tctattacct acaaaatgga agggacatgt atgttgatca ggaactggac
3121 ataaaccgtt tatctgatta cgacgtcgat gccattgtac cccaatcctt tttgaaggac
3181 gattcaatcg acaataaagt gcttacacgc tcggataaga accgagggaa aagtgacaat
3241 gttccaagcg aggaagtcgt aaagaaaatg aagaactatt ggcggcagct cctaaatgcg
3301 aaactgataa cgcaaagaaa gttcgataac ttaactaaag ctgagagggg tggcttgtct
3361 gaacttgaca aggccggatt tattaaacgt cagctcgtgg aaacccgcca aatcacaaag
3421 catgttgcac agatactaga ttcccgaatg aatacgaaat acgacgagaa cgataagctg
3481 attcgggaag tcaaagtaat cactttaaag tcaaaattgg tgtcggactt cagaaaggat
3541 tttcaattct ataaagttag ggagataaat aactaccacc atgcgcacga cgcttatctt
3601 aatgccgtcg tagggaccgc actcattaag aaatacccga agctagaaag tgagtttgtg
3661 tatggtgatt acaaagttta tgacgtccgt aagatgatcg cgaaaagcga acaggagata
3721 ggcaaggcta cagccaaata cttcttttat tctaacatta tgaatttctt taagacggaa
3781 atcactctgg caaacggaga gatacgcaaa cgacctttaa ttgaaaccaa tggggagaca
3841 ggtgaaatcg tatgggataa gggccgggac ttcgcgacgg tgagaaaagt tttgtccatg
3901 ccccaagtca acatagtaaa gaaaactgag gtgcagaccg gagggttttc aaaggaatcg
3961 attcttccaa aaaggaatag tgataagctc atcgctcgta aaaaggactg ggacccgaaa
4021 aagtacggtg gcttcgatag ccctacagtt gcctattctg tcctagtagt ggcaaaagtt
4081 gagaagggaa aatccaagaa actgaagtca gtcaaagaat tattggggat aacgattatg
4141 gagcgctcgt cttttgaaaa gaaccccatc gacttccttg aggcgaaagg ttacaaggaa
4201 gtaaaaaagg atctcataat taaactacca aagtatagtc tgtttgagtt agaaaatggc
4261 cgaaaacgga tgttggctag cgccggagag cttcaaaagg ggaacgaact cgcactaccg
4321 tctaaatacg tgaatttcct gtatttagcg tcccattacg agaagttgaa aggttcacct
4381 gaagataacg aacagaagca actttttgtt gagcagcaca aacattatct cgacgaaatc
4441 atagagcaaa tttcggaatt cagtaagaga gtcatcctag ctgatgccaa tctggacaaa
4501 gtattaagcg catacaacaa gcacagggat aaacccatac gtgagcaggc ggaaaatatt
4561 atccatttgt ttactcttac caacctcggc gctccagccg cattcaagta ttttgacaca
4621 acgatagatc gcaaacgata cacttctacc aaggaggtgc tagacgcgac actgattcac
4681 caatccatca cgggattata tgaaactcgg atagatttgt cacagcttgg gggtgacgga
4741 tcccccaaga agaagaggaa agtctga.
```

In certain embodiments, the dCas9 of the disclosure comprises a dCas9 isolated or derived from Staphyloccocus pyogenes. In certain embodiments, the dCas9 comprises a dCas9 with substitutions at positions 10 and 840 of the amino acid sequence of the dCas9, which inactivate the catalytic site. In certain embodiments, these substitutions are D10A and H840A. In certain embodiments, the “X” residue at position 1 of the dCas9 sequence is a methionine (M). In certain embodiments, the amino acid sequence of the dCas9 comprises the sequence of:

```
(SEQ ID NO: 14498)
      1 XDKKYSIGLA IGTNSVGWAV ITDEYKVPSK KFKVLGNTDR HSIKKNLIGA LLFDSGETAE
    61 ATRLKRTARR RYTRRKNRIC YLQEIFSNEM AKVDDSFFHR LEESFLVEED KKHERHPIFG
  121 NIVDEVAYHE KYPTIYHLRK KLVDSTDKAD LRLIYLALAH MIKFRGHFLI EGDLNPDNSD
  181 VDKLFIQLVQ TYNQLFEENP INASGVDAKA ILSARLSKSR RLENLIAQLP GEKKNGLFGN
  241 LIALSLGLTP NFKSNFDLAE DAKLQLSKDT YDDDLDNLLA QIGDQYADLF LAAKNLSDAI
  301 LLSDILRVNT EITKAPLSAS MIKRYDEHHQ DLTLLKALVR QQLPEKYKEI FFDQSKNGYA
  361 GYIDGGASQE EFYKFIKPIL EKMDGTEELL VKLNREDLLR KQRTFDNGSI PHQIHLGELH
  421 AILRRQEDFY PFLKDNREKI EKILTFRIPY YVGPLARGNS RFAWMTRKSE ETITPWNFEE
  481 VVDKGASAQS FIERMTNFDK NLPNEKVLPK HSLLYEYFTV YNELTKVKYV TEGMRKPAFL
  541 SGEQKKAIVD LLFKTNRKVT VKQLKEDYFK KIECFDSVEI SGVEDRFNAS LGTYHDLLKI
  601 IKDKDFLDNE ENEDILEDIV LTLTLFEDRE MIEERLKTYA HLFDDKVMKQ LKRRRYTGWG
  661 RLSRKLINGI RDKQSGKTIL DFLKSDGFAN RNFMQLIHDD SLTFKEDIQK AQVSGQGDSL
  721 HEHIANLAGS PAIKKGILQT VKVVDELVKV MGRHKPENIV IEMARENQTT QKGQKNSRER
  781 MKRIEEGIKE LGSQILKEHP VENTQLQNEK LYLYYLQNGR DMYVDQELDI NRLSDYDVDA
  841 IVPQSFLKDD SIDNKVLTRS DKNRGKSDNV PSEEVVKKMK NYWRQLLNAK LITQRKFDNL
  901 TKAERGGLSE LDKAGFIKRQ LVETRQITKH VAQILDSRMN TKYDENDKLI REVKVITLKS
  961 KLVSDFRKDF QFYKVREINN YHHAHDAYLN AVVGTALIKK YPKLESEFVY GDYKVYDVRK
1021 MIAKSEQEIG KATAKYFFYS NIMNFFKTEI TLANGEIRKR PLIETNGETG EIVWDKGRDF
1081 ATVRKVLSMP QVNIVKKTEV QTGGFSKESI LPKRNSDKLI ARKKDWDPKK YGGFDSPTVA
1141 YSVLVVAKVE KGKSKKLKSV KELLGITIME RSSFEKNPID FLEAKGYKEV KKDLIIKLPK
1201 YSLFELENGR KRMLASAGEL QKGNELALPS KYVNFLYLAS HYEKLKGSPE DNEQKQLFVE
1261 QHKHYLDEII EQISEFSKRV ILADANLDKV LSAYNKHRDK PIREQAENII HLFTLTNLGA
1321 PAAFKYFDTT IDRKRYTSTK EVLDATLIHQ SITGLYETRI DLSQLGGD.
```

In certain embodiments, the dCas9 of the disclosure comprises a dCas9 isolated or derived from Staphylococcus aureus. In certain embodiments, the dCas9 comprises a dCas9 with substitutions at positions 10 and 580 of the amino acid sequence of the dCas9 which inactivate the catalytic site. In certain embodiments, these substitutions are D10A and N580A. In certain embodiments, the dCas9 is a small and inactive Cas9 (dSaCas9). In certain embodiments, the amino acid sequence of the dSaCas9 comprises the sequence of:

```
(SEQ ID NO: 14658)
      1 mkrnyilglA igitsvgygi idyetrdvid agvrlfkean vennegrrsk rgarrlkrrr
    61 rhriqrvkkl lfdynlltdh selsginpye arvkglsqkl seeefsaall hlakrrgvhn
  121 vneveedtgn elstkeqisr nskaleekyv aelqlerlkk dgevrgsinr fktsdyvkea
  181 kqllkvqkay hqldqsfidt yidlletrrt yyegpgegsp fgwkdikewy emlmghctyf
  241 peelrsvkya ynadlynaln dlnnlvitrd enekleyyek fqiienvfkq kkkptlkqia
  301 keilvneedi kgyrvtstgk peftnlkvyh dikditarke iienaelldq iakiltiyqs
  361 sediqeeltn lnseltqeei eqisnlkgyt gthnlslkai nlildelwht ndnqiaifnr
  421 lklvpkkvd1 sqqkeipttl vddfilspvv krsfiqsikv inaiikkygl pndiiielar
  481 eknskdaqkm inemqkrnrq tnerieeiir ttgkenakyl iekiklhdmq egkclyslea
  541 ipledllnnp fnyevdhiip rsysfdnsfn nkvlvkqeeA skkgnrtpfq ylsssdskis
  601 yetfkkhiln lakgkgrisk tkkeylleer dinrfsvqkd finrnlvdtr yatrglmnll
  661 rsyfrvnnld vkvksinggf tsflrrkwkf kkernkgykh haedaliian adfifkewkk
  721 ldkakkvmen qmfeekqaes mpeieteqey keifitphqi khikdfkdyk yshrvdkkpn
  781 relindtlys trkddkgntl ivnnlnglyd kdndklkkli nkspekllmy hhdpqtyqkl
  841 klimeqygde knplykyyee tgnyltkysk kdngpvikki kyygnklnah lditddypns
  901 rnkvvklslk pyrfdvyldn gvykfvtvkn ldvikkenyy evnskcyeea kklkkisnqa
  961 efiasfynnd likingelyr vigvnndlln rievnmidit yreylenmnd krppriikti
1021 asktqsikky stdilgnlye vkskkhpqii kkg.
```

In certain embodiments of the gene editing systems described herein, the nuclease may comprise, consist essentially of or consist of, a homodimer or a heterodimer. Nuclease domains of the disclosure may comprise, consist essentially of or consist of a nuclease domain isolated, derived or recombined from a transcription-activator-like effector nuclease (TALEN). TALENs are transcription factors with programmable DNA binding domains that provide a means to create designer proteins that bind to pre-determined DNA sequences or individual nucleic acids. Modular DNA binding domains have been identified in transcriptional activator-like (TAL) proteins, or, more specifically, transcriptional activator-like effector nucleases (TALENs), thereby allowing for the de novo creation of synthetic transcription factors that bind to DNA sequences of interest and, if desirable, also allowing a second domain present on the protein or polypeptide to perform an activity related to DNA. TAL proteins have been derived from the organisms Xanthomonas and Ralstonia.

In certain embodiments of the gene editing systems described herein, the nuclease domain may comprise, consist essentially of or consist of a nuclease domain isolated, derived or recombined from a TALEN and a type IIS endonuclease. In certain embodiments of the disclosure, the type IIS endonuclease may comprise, consist essentially of or consist of AciI, Mn1I, AlwI, BbvI, BccI, BceAI, BsmAI, BsmFI, BspCNI, BsrI, BtsCI, HgaI, HphI, HpyAV, MbolI, My1I, PleI, SfaNI, AcuI, BciVI, BfuAI, BmgBI, BmrI, BpmI, BpuEI, BsaI, BseRI, BsgI, BsmI, BspMI, BsrBI, BsrBI, BsrDI, BtgZI, BtsI, EarI, EciI, MmeI, NmeAIII, BbvCI, Bpu10I, BspQI, SapI, BaeI, BsaXI, CspCI, BfiI, MboII, Acc36I, FokI or Clo051. In certain embodiments of the disclosure, the type IIS endonuclease may comprise, consist essentially of or consist of Clo051 (SEQ ID NO: 14503).

In certain embodiments of the gene editing systems described herein, the nuclease domain of may comprise, consist essentially of or consist of a nuclease domain isolated, derived or recombined from a zinc finger nuclease (ZFN) and a type IIS endonuclease. In certain embodiments of the disclosure, the type IIS endonuclease may comprise, consist essentially of or consist of AciI, Mn1I, AlwI, BbvI, BccI, BceAI, BsmAI, BsmFI, BspCNI, BsrI, BtsCI, HgaI, HphI, HpyAV, Mbo1I, My1I, PleI, SfaNI, AcuI, BciVI, BfuAI, BmgBI, BmrI, BpmI, BpuEI, BsaI, BseRI, BsgI, BsmI, BspMI, BsrBI, BsrBI, BsrDI, BtgZI, BtsI, EarI, EciI, MmeI, NmeAIII, BbvCI, Bpu10I, BspQI, SapI, BaeI, BsaXI, CspCI, BfiI, MboII, Acc36I, FokI or Clo051. In certain embodiments of the disclosure, the type IIS endonuclease may comprise, consist essentially of or consist of Clo051 (SEQ ID NO: 14503).

In certain embodiments of the gene editing systems described herein, the DNA binding domain and the nuclease domain may be covalently linked. For example, a fusion protein may comprise the DNA binding domain and the nuclease domain. In certain embodiments of the genomic editing compositions or constructs of the disclosure, the DNA binding domain and the nuclease domain may be operably linked through a non-covalent linkage.

### Therapeutic Proteins

In certain embodiments of the composition and methods of the disclosure, modified immune or immune precursor cells express therapeutic proteins. Therapeutic proteins of the disclosure include secreted proteins. Preferably, in a therapeutic context, the therapeutic protein is a human protein, including a secreted human protein. When expressed or secreted by immune or immune precursor cells of the disclosure, the combination comprising the immune or immune precursor cell and the therapeutic protein secreted therefrom may be considered a monotherapy. However, the immune or immune precursor cells of the disclosure may be administered as a combination therapy with a second agent. Human therapeutic proteins of the disclosure include, but are not limited to, those provided at Table 1.

TABLE 1
Exemplary Human Secreted Proteins

| Gene Name | Gene Description | Protein SEQ ID NO |
|---|---|---|
| A1BG | Alpha-1-B glycoprotein | SEQ ID NOS: 1-2 |
| A2M | Alpha-2-macroglobulin | SEQ ID NOS: 3-6 |
| A2ML1 | Alpha-2-macroglobulin-like 1 | SEQ ID NOS: 7-12 |
| A4GNT | Alpha-1,4-N-acetylglucosaminyltransferase | SEQ ID NO: 13 |
| AADACL2 | Arylacetamide deacetylase-like 2 | SEQ ID NOS: 14-15 |
| AANAT | Aralkylamine N-acetyltransferase | SEQ ID NOS: 16-19 |
| ABCG1 | ATP-binding cassette, sub-family G (WHITE), | SEQ ID NOS: 20-26 |
| | member 1 | |
| ABHD1 | Abhydrolase domain containing 1 | SEQ ID NOS: 27-31 |
| ABHD10 | Abhydrolase domain containing 10 | SEQ ID NOS: 32-35 |
| ABHD14A | Abhydrolase domain containing 14A | SEQ ID NOS: 36-40 |
| ABHD15 | Abhydrolase domain containing 15 | SEQ ID NO: 41 |
| ABI3BP | ABI family, member 3 (NESH) binding protein | SEQ ID NOS: 42-63 |
| FAM175A | Family with sequence similarity 175, member A | SEQ ID NOS: 64-71 |
| LA16c- | | SEQ ID NO: 72 |
| 380H5.3 | | |
| AC008641.1 | | SEQ ID NO: 73 |
| CTB- | | SEQ ID NOS: 74-75 |
| 601318.6 | | |
| AC009133.22 | | SEQ ID NO: 76 |
| AC009491.2 | | SEQ ID NO: 77 |
| RP11- | | SEQ ID NOS: 78-80 |
| 977G19.10 | | |
| CTD- | | SEQ ID NOS: 81-84 |
| 2370N5.3 | | |
| RP11- | | SEQ ID NOS: 85-87 |
| 196G11.1 | | |
| AC136352.5 | | SEQ ID NO: 88 |
| RP11- | | SEQ ID NO: 89 |
| 812E19.9 | | |
| AC145212.4 | MaFF-interacting protein | SEQ ID NO: 90 |
| AC233755.1 | | SEQ ID NO: 91 |
| AC011513.3 | | SEQ ID NOS: 92-93 |
| ACACB | Acetyl-CoA carboxylase beta | SEQ ID NOS: 94-100 |
| ACAN | Aggrecan | SEQ ID NOS: 101-108 |
| ACE | Angiotensin I converting enzyme | SEQ ID NOS: 109-121 |
| ACHE | Acetylcholinesterase (Yt blood group) | SEQ ID NOS: 122-134 |
| ACP2 | Acid phosphatase 2, lysosomal | SEQ ID NOS: 135-142 |
| ACP5 | Acid phosphatase 5, tartrate resistant | SEQ ID NOS: 143-151 |
| ACP6 | Acid phosphatase 6, lysophosphatidic | SEQ ID NOS: 152-158 |
| PAPL | Iron/zinc purple acid phosphatase-like protein | SEQ ID NOS: 159-162 |
| ACPP | Acid phosphatase, prostate | SEQ ID NOS: 163-167 |
| ACR | Acrosin | SEQ ID NOS: 168-169 |
| ACRBP | Acrosin binding protein | SEQ ID NOS: 170-174 |
| ACRV1 | Acrosomal vesicle protein 1 | SEQ ID NOS: 175-178 |
| ACSF2 | Acyl-CoA synthetase family member 2 | SEQ ID NOS: 179-187 |
| ACTL10 | Actin-like 10 | SEQ ID NO: 188 |
| ACVR1 | Activin A receptor, type I | SEQ ID NOS: 189-197 |
| ACVR1C | Activin A receptor, type IC | SEQ ID NOS: 198-201 |
| ACVRL1 | Activin A receptor type II-like 1 | SEQ ID NOS: 202-207 |
| ACYP1 | Acylphosphatase 1, erythrocyte (common) type | SEQ ID NOS: 208-213 |
| ACYP2 | Acylphosphatase 2, muscle type | SEQ ID NOS: 214-221 |
| CECR1 | Cat eye syndrome chromosome region, candidate 1 | SEQ ID NOS: 222-229 |
| ADAM10 | ADAM metallopeptidase domain 10 | SEQ ID NOS: 230-237 |
| ADAM12 | ADAM metallopeptidase domain 12 | SEQ ID NOS: 238-240 |
| ADAM15 | ADAM metallopeptidase domain 15 | SEQ ID NOS: 241-252 |
| ADAM17 | ADAM metallopeptidase domain 17 | SEQ ID NOS: 253-255 |
| ADAM18 | ADAM metallopeptidase domain 18 | SEQ ID NOS: 256-260 |
| ADAM22 | ADAM metallopeptidase domain 22 | SEQ ID NOS: 261-269 |
| ADAM28 | ADAM metallopeptidase domain 28 | SEQ ID NOS: 270-275 |
| ADAM29 | ADAM metallopeptidase domain 29 | SEQ ID NOS: 276-284 |
| ADAM32 | ADAM metallopeptidase domain 32 | SEQ ID NOS: 285-291 |
| ADAM33 | ADAM metallopeptidase domain 33 | SEQ ID NOS: 292-296 |
| ADAM7 | ADAM metallopeptidase domain 7 | SEQ ID NOS: 297-300 |
| ADAM8 | ADAM metallopeptidase domain 8 | SEQ ID NOS: 301-305 |
| ADAM9 | ADAM metallopeptidase domain 9 | SEQ ID NOS: 306-311 |
| ADAMDEC1 | ADAM-like, decysin 1 | SEQ ID NOS: 312-314 |
| ADAMTS1 | ADAM metallopeptidase with thrombospondin type | SEQ ID NOS: 315-318 |
| | 1 motif, 1 | |
| ADAMTS10 | ADAM metallopeptidase with thrombospondin type | SEQ ID NOS: 319-324 |
| | 1 motif, 10 | |
| ADAMTS12 | ADAM metallopeptidase with thrombospondin type | SEQ ID NOS: 325-327 |
| | 1 motif, 12 | |
| ADAMTS13 | ADAM metallopeptidase with thrombospondin type | SEQ ID NOS: 328-335 |
| | 1 motif, 13 | |
| ADAMTS14 | ADAM metallopeptidase with thrombospondin type | SEQ ID NOS: 336-337 |
| | 1 motif, 14 | |
| ADAMTS15 | ADAM metallopeptidase with thrombospondin type | SEQ ID NO: 338 |
| | 1 motif, 15 | |
| ADAMTS16 | ADAM metallopeptidase with thrombospondin type | SEQ ID NOS: 339-340 |
| | 1 motif, 16 | |
| ADAMTS17 | ADAM metallopeptidase with thrombospondin type | SEQ ID NOS: 341-344 |
| | 1 motif, 17 | |
| ADAMTS18 | ADAM metallopeptidase with thrombospondin type | SEQ ID NOS: 345-348 |
| | 1 motif, 18 | |
| ADAMTS19 | ADAM metallopeptidase with thrombospondin type | SEQ ID NOS: 349-352 |
| | 1 motif, 19 | |
| ADAMTS2 | ADAM metallopeptidase with thrombospondin type | SEQ ID NOS: 353-355 |
| | 1 motif, 2 | |
| ADAMTS20 | ADAM metallopeptidase with thrombospondin type | SEQ ID NOS: 356-359 |
| | 1 motif, 20 | |
| ADAMTS3 | ADAM metallopeptidase with thrombospondin type | SEQ ID NOS: 360-361 |
| | 1 motif, 3 | |
| ADAMTS5 | ADAM metallopeptidase with thrombospondin type | SEQ ID NO: 362 |
| | 1 motif, 5 | |
| ADAMTS6 | ADAM metallopeptidase with thrombospondin type | SEQ ID NOS: 363-364 |
| | 1 motif, 6 | |
| ADAMTS7 | ADAM metallopeptidase with thrombospondin type | SEQ ID NO: 365 |
| | 1 motif, 7 | |
| ADAMTS8 | ADAM metallopeptidase with thrombospondin type | SEQ ID NO: 366 |
| | 1 motif, 8 | |
| ADAMTS9 | ADAM metallopeptidase with thrombospondin type | SEQ ID NOS: 367-371 |
| | 1 motif, 9 | |
| ADAMTSL1 | ADAMTS-like 1 | SEQ ID NOS: 372-382 |
| ADAMTSL2 | ADAMTS-like 2 | SEQ ID NOS: 383-385 |
| ADAMTSL3 | ADAMTS-like 3 | SEQ ID NOS: 386-387 |
| ADAMTSL4 | ADAMTS-like 4 | SEQ ID NOS: 388-391 |
| ADAMTSL5 | ADAMTS-like 5 | SEQ ID NOS: 392-397 |
| ADCK1 | AarF domain containing kinase 1 | SEQ ID NOS: 398-402 |
| ADCYAP1 | Adenylate cyclase activating polypeptide 1 | SEQ ID NOS: 403-404 |
| | (pituitary) | |
| ADCYAP1R1 | Adenylate cyclase activating polypeptide 1 | SEQ ID NOS: 405-411 |
| | (pituitary) receptor type I | |
| ADGRA3 | Adhesion G protein-coupled receptor A3 | SEQ ID NOS: 412-416 |
| ADGRB2 | Adhesion G protein-coupled receptor B2 | SEQ ID NOS: 417-425 |
| ADGRD1 | Adhesion G protein-coupled receptor D1 | SEQ ID NOS: 426-431 |
| ADGRE3 | Adhesion G protein-coupled receptor E3 | SEQ ID NOS: 432-436 |
| ADGRE5 | Adhesion G protein-coupled receptor E5 | SEQ ID NOS: 437-442 |
| ADGRF1 | Adhesion G protein-coupled receptor F1 | SEQ ID NOS: 443-447 |
| ADGRG1 | Adhesion G protein-coupled receptor G1 | SEQ ID NOS: 448-512 |
| ADGRG5 | Adhesion G protein-coupled receptor G5 | SEQ ID NOS: 513-515 |
| ADGRG6 | Adhesion G protein-coupled receptor G6 | SEQ ID NOS: 516-523 |
| ADGRV1 | Adhesion G protein-coupled receptor V1 | SEQ ID NOS: 524-540 |
| ADI1 | Acireductone dioxygenase 1 | SEQ ID NOS: 541-543 |
| ADIG | Adipogenin | SEQ ID NOS: 544-547 |
| ADIPOQ | Adiponectin, C1Q and collagen domain containing | SEQ ID NOS: 548-549 |
| ADM | Adrenomedullin | SEQ ID NOS: 550-557 |
| ADM2 | Adrenomedullin 2 | SEQ ID NOS: 558-559 |
| ADM5 | Adrenomedullin 5 (putative) | SEQ ID NO: 560 |
| ADPGK | ADP-dependent glucokinase | SEQ ID NOS: 561-570 |
| ADPRHL2 | ADP-ribosylhydrolase like 2 | SEQ ID NO: 571 |
| AEBP1 | AE binding protein 1 | SEQ ID NOS: 572-579 |
| LACE1 | Lactation elevated 1 | SEQ ID NOS: 580-583 |
| AFM | Afamin | SEQ ID NO: 584 |
| AFP | Alpha-fetoprotein | SEQ ID NOS: 585-586 |
| AGA | Aspartylglucosaminidase | SEQ ID NOS: 587-589 |
| AGER | Advanced glycosylation end product-specific | SEQ ID NOS: 590-600 |
| | receptor | |
| AGK | Acylglycerol kinase | SEQ ID NOS: 601-606 |
| AGPS | Alkylglycerone phosphate synthase | SEQ ID NOS: 607-610 |
| AGR2 | Anterior gradient 2, protein disulphide isomerase | SEQ ID NOS: 611-614 |
| | family member | |
| AGR3 | Anterior gradient 3, protein disulphide isomerase | SEQ ID NOS: 615-617 |
| | family member | |
| AGRN | Agrin | SEQ ID NOS: 618-621 |
| AGRP | Agouti related neuropeptide | SEQ ID NO: 622 |
| AGT | Angiotensinogen (serpin peptidase inhibitor, clade A, | SEQ ID NO: 623 |
| | member 8) | |
| AGTPBP1 | ATP/GTP binding protein 1 | SEQ ID NOS: 624-627 |
| AGTRAP | Angiotensin 11 receptor-associated protein | SEQ ID NOS: 628-635 |
| AHCYL2 | Adenosylhomocysteinase-like 2 | SEQ ID NOS: 636-642 |
| AHSG | Alpha-2-HS-glycoprotein | SEQ ID NOS: 643-644 |
| AIG1 | Androgen-induced 1 | SEQ ID NOS: 645-653 |
| AK4 | Adenylate kinase 4 | SEQ ID NOS: 654-657 |
| AKAP10 | A kinase (PRKA) anchor protein 10 | SEQ ID NOS: 658-666 |
| AKR1C1 | Aldo-keto reductase family 1, member C1 | SEQ ID NOS: 667-669 |
| RP4- | | SEQ ID NOS: 670-672 |
| 576H24.4 | | |
| SERPINA3 | Serpin peptidase inhibitor, clade A (alpha-1 | SEQ ID NO: 673 |
| | antiproteinase, antitrypsin), member 3 | |
| RP11-14J7.7 | | SEQ ID NOS: 674-675 |
| RP11- | | SEQ ID NO: 676 |
| 903H12.5 | | |
| AL356289.1 | | SEQ ID NO: 677 |
| AL589743.1 | | SEQ ID NO: 678 |
| XXbac- | | SEQ ID NOS: 679-680 |
| BPG116M5.17 | | |
| XXbac- | | SEQ ID NO: 681 |
| BPG181M17.5 | | |
| XXbac- | | SEQ ID NO: 682 |
| BPG32J3.20 | | |
| RP11- | | SEQ ID NO: 683 |
| 350O14.18 | | |
| ALAS2 | 5′-aminolevulinate synthase 2 | SEQ ID NOS: 684-691 |
| ALB | Albumin | SEQ ID NOS: 692-701 |
| ALDH9A1 | Aldehyde dehydrogenase 9 family, member A1 | SEQ ID NO: 702 |
| ALDOA | Aldolase A, fructose-bisphosphate | SEQ ID NOS: 703-717 |
| ALG1 | ALG1, chitobiosyldiphosphodolichol beta- | SEQ ID NOS: 718-723 |
| | mannosyltransferase | |
| ALG5 | ALG5, dolichyl-phosphate beta-glucosyltransferase | SEQ ID NOS: 724-725 |
| ALG9 | ALG9, alpha-1,2-mannosyltransferase | SEQ ID NOS: 726-736 |
| FAM150A | Family with sequence similarity 150, member A | SEQ ID NOS: 737-738 |
| FAM150B | Family with sequence similarity 150, member B | SEQ ID NOS: 739-745 |
| ALKBH1 | AlkB homolog 1, histone H2A dioxygenase | SEQ ID NOS: 746-748 |
| ALKBH5 | AlkB homolog 5, RNA demethylase | SEQ ID NOS: 749-750 |
| ALPI | Alkaline phosphatase, intestinal | SEQ ID NOS: 751-752 |
| ALPL | Alkaline phosphatase, liver/bone/kidney | SEQ ID NOS: 753-757 |
| ALPP | Alkaline phosphatase, placental | SEQ ID NO: 758 |
| ALPPL2 | Alkaline phosphatase, placental-like 2 | SEQ ID NO: 759 |
| AMBN | Ameloblastin (enamel matrix protein) | SEQ ID NOS: 760-762 |
| AMBP | Alpha-1-microglobulin/bikunin precursor | SEQ ID NOS: 763-765 |
| AMELX | Amelogenin, X-linked | SEQ ID NOS: 766-768 |
| AMELY | Amelogenin, Y-linked | SEQ ID NOS: 769-770 |
| AMH | Anti-Mullerian hormone | SEQ ID NO: 771 |
| AMPD1 | Adenosine monophosphate deaminase 1 | SEQ ID NOS: 772-774 |
| AMTN | Amelotin | SEQ ID NOS: 775-776 |
| AMY1A | Amylase, alpha 1A (salivary) | SEQ ID NOS: 777-779 |
| AMY1B | Amylase, alpha 1B (salivary) | SEQ ID NOS: 780-783 |
| AMY1C | Amylase, alpha 1C (salivary) | SEQ ID NO: 784 |
| AMY2A | Amylase, alpha 2A (pancreatic) | SEQ ID NOS: 785-787 |
| AMY2B | Amylase, alpha 2B (pancreatic) | SEQ ID NOS: 788-792 |
| ANG | Angiogenin, ribonuclease, RNase A family, 5 | SEQ ID NOS: 793-794 |
| ANGEL1 | Angel homolog 1 (Drosophila) | SEQ ID NOS: 795-798 |
| ANGPT1 | Angiopoietin 1 | SEQ ID NOS: 799-803 |
| ANGPT2 | Angiopoietin 2 | SEQ ID NOS: 804-807 |
| ANGPT4 | Angiopoietin 4 | SEQ ID NO: 808 |
| ANGPTL1 | Angiopoietin-like 1 | SEQ ID NOS: 809-811 |
| ANGPTL2 | Angiopoietin-like 2 | SEQ ID NOS: 812-813 |
| ANGPTL3 | Angiopoietin-like 3 | SEQ ID NO: 814 |
| ANGPTL4 | Angiopoietin-like 4 | SEQ ID NOS: 815-822 |
| ANGPTL5 | Angiopoietin-like 5 | SEQ ID NOS: 823-824 |
| ANGPTL6 | Angiopoietin-like 6 | SEQ ID NOS: 825-827 |
| ANGPTL7 | Angiopoietin-like 7 | SEQ ID NO: 828 |
| C19orf80 | Chromosome 19 open reading frame 80 | SEQ ID NOS: 829-832 |
| ANK1 | Ankyrin 1, erythrocytic | SEQ ID NOS: 833-843 |
| ANKDD1A | Ankyrin repeat and death domain containing 1A | SEQ ID NOS: 844-850 |
| ANKRD54 | Ankyrin repeat domain 54 | SEQ ID NOS: 851-859 |
| ANKRD60 | Ankyrin repeat domain 60 | SEQ ID NO: 860 |
| ANO7 | Anoctamin 7 | SEQ ID NOS: 861-864 |
| ANOS1 | Anosmin 1 | SEQ ID NO: 865 |
| ANTXR1 | Anthrax toxin receptor 1 | SEQ ID NOS: 866-869 |
| AOAH | Acyloxyacyl hydrolase (neutrophil) | SEQ ID NOS: 870-874 |
| AOC1 | Amine oxidase, copper containing 1 | SEQ ID NOS: 875-880 |
| AOC2 | Amine oxidase, copper containing 2 (retina-specific) | SEQ ID NOS: 881-882 |
| AOC3 | Amine oxidase, copper containing 3 | SEQ ID NOS: 883-889 |
| AP000721.4 | | SEQ ID NO: 890 |
| APBB1 | Amyloid beta (A4) precursor protein-binding, family | SEQ ID NOS: 891-907 |
| | B, member 1 (Fe65) | |
| APCDD1 | Adenomatosis polyposis coli down-regulated 1 | SEQ ID NOS: 908-913 |
| APCS | Amyloid P component, serum | SEQ ID NO: 914 |
| APELA | Apelin receptor early endogenous ligand | SEQ ID NOS: 915-917 |
| APLN | Apelin | SEQ ID NO: 918 |
| APLP2 | Amyloid beta (A4) precursor-like protein 2 | SEQ ID NOS: 919-928 |
| APOA1 | Apolipoprotein A-I | SEQ ID NOS: 929-933 |
| APOA2 | Apolipoprotein A-II | SEQ ID NOS: 934-942 |
| APOA4 | Apolipoprotein A-IV | SEQ ID NO: 943 |
| APOA5 | Apolipoprotein A-V | SEQ ID NOS: 944-946 |
| APOB | Apolipoprotein B | SEQ ID NOS: 947-948 |
| APOC1 | Apolipoprotein C-I | SEQ ID NOS: 949-957 |
| APOC2 | Apolipoprotein C-II | SEQ ID NOS: 958-962 |
| APOC3 | Apolipoprotein C-III | SEQ ID NOS: 963-966 |
| APOC4 | Apolipoprotein C-IV | SEQ ID NOS: 967-968 |
| APOC4- | APOC4-APOC2 readthrough (NMD candidate) | SEQ ID NOS: 969-970 |
| APOC2 | | |
| APOD | Apolipoprotein D | SEQ ID NOS: 971-974 |
| APOE | Apolipoprotein E | SEQ ID NOS: 975-978 |
| APOF | Apolipoprotein F | SEQ ID NO: 979 |
| APOH | Apolipoprotein H (beta-2-glycoprotein I) | SEQ ID NOS: 980-983 |
| APOL1 | Apolipoprotein L, 1 | SEQ ID NOS: 984-994 |
| APOL3 | Apolipoprotein L, 3 | SEQ ID NOS: 995-1009 |
| APOM | Apolipoprotein M | SEQ ID NOS: 1010-1012 |
| APOOL | Apolipoprotein O-like | SEQ ID NOS: 1013-1015 |
| ARCN1 | Archain 1 | SEQ ID NOS: 1016-1020 |
| ARFIP2 | ADP-ribosylation factor interacting protein 2 | SEQ ID NOS: 1021-1027 |
| ARHGAP36 | Rho GTPase activating protein 36 | SEQ ID NOS: 1028-1033 |
| HMHA1 | Histocompatibility (minor) HA-1 | SEQ ID NOS: 1034-1042 |
| ARHGAP6 | Rho GTPase activating protein 6 | SEQ ID NOS: 1043-1048 |
| ARHGEF4 | Rho guanine nucleotide exchange factor (GEF) 4 | SEQ ID NOS: 1049-1059 |
| ARL16 | ADP-ribosylation factor-like 16 | SEQ ID NOS: 1060-1068 |
| ARMC5 | Armadillo repeat containing 5 | SEQ ID NOS: 1069-1075 |
| ARNTL | Aryl hydrocarbon receptor nuclear translocator-like | SEQ ID NOS: 1076-1090 |
| ARSA | Arylsulfatase A | SEQ ID NOS: 1091-1096 |
| ARSB | Arylsulfatase B | SEQ ID NOS: 1097-1100 |
| ARSE | Arylsulfatase E (chondrodysplasia punctata 1) | SEQ ID NOS: 1101-1104 |
| ARSG | Arylsulfatase G | SEQ ID NOS: 1105-1108 |
| ARSI | Arylsulfatase family, member I | SEQ ID NOS: 1109-1111 |
| ARSK | Arylsulfatase family, member K | SEQ ID NOS: 1112-1116 |
| ART3 | ADP-ribosyltransferase 3 | SEQ ID NOS: 1117-1124 |
| ART4 | ADP-ribosyltransferase 4 (Dombrock blood group) | SEQ ID NOS: 1125-1128 |
| ART5 | ADP-ribosyltransferase 5 | SEQ ID NOS: 1129-1133 |
| ARTN | Artemin | SEQ ID NOS: 1134-1144 |
| ASAH1 | N-acylsphingosine amidohydrolase (acid | SEQ ID NOS: 1145-1195 |
| | ceramidase) 1 | |
| ASAH2 | N-acylsphingosine amidohydrolase (non-lysosomal | SEQ ID NOS: 1196-1201 |
| | ceramidase) 2 | |
| ASCL1 | Achaete-scute family bHLH transcription factor 1 | SEQ ID NO: 1202 |
| ASIP | Agouti signaling protein | SEQ ID NOS: 1203-1204 |
| ASPN | Asporin | SEQ ID NOS: 1205-1206 |
| ASTL | Astacin-like metallo-endopeptidase (M12 family) | SEQ ID NO: 1207 |
| ATAD5 | ATPase family, AAA domain containing 5 | SEQ ID NOS: 1208-1209 |
| ATAT1 | Alpha tubulin acetyltransferase 1 | SEQ ID NOS: 1210-1215 |
| ATG2A | Autophagy related 2A | SEQ ID NOS: 1216-1218 |
| ATG5 | Autophagy related 5 | SEQ ID NOS: 1219-1227 |
| ATMIN | ATM interactor | SEQ ID NOS: 1228-1231 |
| ATP13A1 | ATPase type 13A1 | SEQ ID NOS: 1232-1234 |
| ATP5F1 | ATP synthase, H+ transporting, mitochondrial Fo | SEQ ID NOS: 1235-1236 |
| | complex, subunit B1 | |
| ATP6AP1 | ATPase, H+ transporting, lysosomal accessory | SEQ ID NOS: 1237-1244 |
| | protein 1 | |
| ATP6AP2 | ATPase, H+ transporting, lysosomal accessory | SEQ ID NOS: 1245-1267 |
| | protein 2 | |
| ATPAF1 | ATP synthase mitochondrial F1 complex assembly | SEQ ID NOS: 1268-1278 |
| | factor 1 | |
| AUH | AU RNA binding protein/enoyl-CoA hydratase | SEQ ID NOS: 1279-1280 |
| AVP | Arginine vasopressin | SEQ ID NO: 1281 |
| AXIN2 | Axin 2 | SEQ ID NOS: 1282-1289 |
| AZGP1 | Alpha-2-glycoprotein 1, zinc-binding | SEQ ID NOS: 1290-1292 |
| AZU1 | Azurocidin 1 | SEQ ID NOS: 1293-1294 |
| B2M | Beta-2-microglobulin | SEQ ID NOS: 1295-1301 |
| B3GALNT1 | Beta-1,3-N-acetylgalactosaminyltransferase 1 | SEQ ID NOS: 1302-1314 |
| | (globoside blood group) | |
| B3GALNT2 | Beta-1,3-N-acetylgalactosaminyltransferase 2 | SEQ ID NOS: 1315-1317 |
| B3GALT1 | UDP-Gal:betaGlcNAc beta 1,3-galactosyltransferase, | SEQ ID NO: 1318 |
| | polypeptide 1 | |
| B3GALT4 | UDP-Gal:betaGlcNAc beta 1,3-galactosyltransferase, | SEQ ID NO: 1319 |
| | polypeptide 4 | |
| B3GALT5 | UDP-Gal:betaGlcNAc beta 1,3-galactosyltransferase, | SEQ ID NOS: 1320-1324 |
| | polypeptide 5 | |
| B3GALT6 | UDP-Gal:betaGal beta 1,3-galactosyltransferase | SEQ ID NO: 1325 |
| | polypeptide 6 | |
| B3GAT3 | Beta-1,3-glucuronyltransferase 3 | SEQ ID NOS: 1326-1330 |
| B3GLCT | Beta 3-glucosyltransferase | SEQ ID NO: 1331 |
| B3GNT3 | UDP-GlcNAc:betaGal beta-1,3-N- | SEQ ID NOS: 1332-1335 |
| | acetylglucosaminyltransferase 3 | |
| B3GNT4 | UDP-GlcNAc:betaGal beta-1,3-N- | SEQ ID NOS: 1336-1339 |
| | acetylglucosaminyltransferase 4 | |
| B3GNT6 | UDP-GlcNAc:betaGal beta-1,3-N- | SEQ ID NOS: 1340-1341 |
| | acetylglucosaminyltransferase 6 | |
| B3GNT7 | UDP-GlcNAc:betaGal beta-1,3-N- | SEQ ID NO: 1342 |
| | acetylglucosaminyltransferase 7 | |
| B3GNT8 | UDP-GlcNAc:betaGal beta-1,3-N- | SEQ ID NO: 1343 |
| | acetylglucosaminyltransferase 8 | |
| B3GNT9 | UDP-GlcNAc:betaGal beta-1,3-N- | SEQ ID NO: 1344 |
| | acetylglucosaminyltransferase 9 | |
| B4GALNT1 | Beta-1,4-N-acetyl-galactosaminyl transferase 1 | SEQ ID NOS: 1345-1356 |
| B4GALNT3 | Beta-1,4-N-acetyl-galactosaminyl transferase 3 | SEQ ID NOS: 1357-1358 |
| B4GALNT4 | Beta-1,4-N-acetyl-galactosaminyl transferase 4 | SEQ ID NOS: 1359-1361 |
| B4GALT4 | UDP-Gal:betaGlcNAc beta 1,4-galactosyltransferase, | SEQ ID NOS: 1362-1375 |
| | polypeptide 4 | |
| B4GALT5 | UDP-Gal:betaGlcNAc beta 1,4-galactosyltransferase, | SEQ ID NO: 1376 |
| | polypeptide 5 | |
| B4GALT6 | UDP-Gal:betaGlcNAc beta 1,4-galactosyltransferase, | SEQ ID NOS: 1377-1380 |
| | polypeptide 6 | |
| B4GAT1 | Beta-1,4-glucuronyltransferase 1 | SEQ ID NO: 1381 |
| B9D1 | B9 protein domain 1 | SEQ ID NOS: 1382-1398 |
| BACE2 | Beta-site APP-cleaving enzyme 2 | SEQ ID NOS: 1399-1401 |
| BAGE5 | B melanoma antigen family, member 5 | SEQ ID NO: 1402 |
| BCAM | Basal cell adhesion molecule (Lutheran blood group) | SEQ ID NOS: 1403-1406 |
| BCAN | Brevican | SEQ ID NOS: 1407-1413 |
| BCAP29 | B-cell receptor-associated protein 29 | SEQ ID NOS: 1414-1426 |
| BCAR1 | Breast cancer anti-estrogen resistance 1 | SEQ ID NOS: 1427-1444 |
| BCHE | Butyrylcholinesterase | SEQ ID NOS: 1445-1449 |
| BCKDHB | Branched chain keto acid dehydrogenase E1, beta | SEQ ID NOS: 1450-1452 |
| | polypeptide | |
| BDNF | Brain-derived neurotrophic factor | SEQ ID NOS: 1453-1470 |
| BGLAP | Bone gamma-carboxyglutamate (gla) protein | SEQ ID NO: 1471 |
| BGN | Biglycan | SEQ ID NOS: 1472-1473 |
| BLVRB | Biliverdin reductase B | SEQ ID NOS: 1474-1478 |
| BMP1 | Bone morphogenetic protein 1 | SEQ ID NOS: 1479-1490 |
| BMP10 | Bone morphogenetic protein 10 | SEQ ID NO: 1491 |
| BMP15 | Bone morphogenetic protein 15 | SEQ ID NO: 1492 |
| BMP2 | Bone morphogenetic protein 2 | SEQ ID NO: 1493 |
| BMP3 | Bone morphogenetic protein 3 | SEQ ID NO: 1494 |
| BMP4 | Bone morphogenetic protein 4 | SEQ ID NOS: 1495-1502 |
| BMP6 | Bone morphogenetic protein 6 | SEQ ID NO: 1503 |
| BMP7 | Bone morphogenetic protein 7 | SEQ ID NOS: 1504-1507 |
| BMP8A | Bone morphogenetic protein 8a | SEQ ID NO: 1508 |
| BMP8B | Bone morphogenetic protein 8b | SEQ ID NO: 1509 |
| BMPER | BMP binding endothelial regulator | SEQ ID NOS: 1510-1513 |
| BNC1 | Basonuclin 1 | SEQ ID NOS: 1514-1515 |
| BOC | BOC cell adhesion associated, oncogene regulated | SEQ ID NOS: 1516-1526 |
| BOD1 | Biorientation of chromosomes in cell division 1 | SEQ ID NOS: 1527-1531 |
| BOLA1 | BolA family member 1 | SEQ ID NOS: 1532-1534 |
| BPI | Bactericidal/permeability-increasing protein | SEQ ID NOS: 1535-1538 |
| BPIFA1 | BPI fold containing family A, member 1 | SEQ ID NOS: 1539-1542 |
| BPIFA2 | BPI fold containing family A, member 2 | SEQ ID NOS: 1543-1544 |
| BPIFA3 | BPI fold containing family A, member 3 | SEQ ID NOS: 1545-1546 |
| BPIFB1 | BPI fold containing family B, member 1 | SEQ ID NOS: 1547-1548 |
| BPIFB2 | BPI fold containing family B, member 2 | SEQ ID NO: 1549 |
| BPIFB3 | BPI fold containing family B, member 3 | SEQ ID NO: 1550 |
| BPIFB4 | BPI fold containing family B, member 4 | SEQ ID NOS: 1551-1552 |
| BPIFB6 | BPI fold containing family B, member 6 | SEQ ID NOS: 1553-1554 |
| BPIFC | BPI fold containing family C | SEQ ID NOS: 1555-1558 |
| BRF1 | BRF1, RNA polymerase III transcription initiation | SEQ ID NOS: 1559-1574 |
| | factor 90 kDa subunit | |
| BRINP1 | Bone morphogenetic protein/retinoic acid inducible | SEQ ID NOS: 1575-1576 |
| | neural-specific 1 | |
| BRINP2 | Bone morphogenetic protein/retinoic acid inducible | SEQ ID NO: 1577 |
| | neural-specific 2 | |
| BRINP3 | Bone morphogenetic protein/retinoic acid inducible | SEQ ID NOS: 1578-1580 |
| | neural-specific 3 | |
| BSG | Basigin (Ok blood group) | SEQ ID NOS: 1581-1591 |
| BSPH1 | Binder of sperm protein homolog 1 | SEQ ID NO: 1592 |
| BST1 | Bone marrow stromal cell antigen 1 | SEQ ID NOS: 1593-1597 |
| BTBD17 | BTB (POZ) domain containing 17 | SEQ ID NO: 1598 |
| BTD | Biotinidase | SEQ ID NOS: 1599-1608 |
| BTN2A2 | Butyrophilin, subfamily 2, member A2 | SEQ ID NOS: 1609-1622 |
| BTN3A1 | Butyrophilin, subfamily 3, member A1 | SEQ ID NOS: 1623-1629 |
| BTN3A2 | Butyrophilin, subfamily 3, member A2 | SEQ ID NOS: 1630-1640 |
| BTN3A3 | Butyrophilin, subfamily 3, member A3 | SEQ ID NOS: 1641-1649 |
| RP4- | Complement factor H-related protein 2 | SEQ ID NO: 1650 |
| 608O15.3 | | |
| C10orf99 | Chromosome 10 open reading frame 99 | SEQ ID NO: 1651 |
| C11orf1 | Chromosome 11 open reading frame 1 | SEQ ID NOS: 1652-1656 |
| C11orf24 | Chromosome 11 open reading frame 24 | SEQ ID NOS: 1657-1659 |
| C11orf45 | Chromosome 11 open reading frame 45 | SEQ ID NOS: 1660-1661 |
| C11orf94 | Chromosome 11 open reading frame 94 | SEQ ID NO: 1662 |
| C12orf10 | Chromosome 12 open reading frame 10 | SEQ ID NOS: 1663-1666 |
| C12orf49 | Chromosome 12 open reading frame 49 | SEQ ID NOS: 1667-1670 |
| C12orf73 | Chromosome 12 open reading frame 73 | SEQ ID NOS: 1671-1680 |
| C12orf76 | Chromosome 12 open reading frame 76 | SEQ ID NOS: 1681-1688 |
| C14orf93 | Chromosome 14 open reading frame 93 | SEQ ID NOS: 1689-1704 |
| C16orf89 | Chromosome 16 open reading frame 89 | SEQ ID NOS: 1705-1707 |
| C16orf90 | Chromosome 16 open reading frame 90 | SEQ ID NOS: 1708-1709 |
| C17orf67 | Chromosome 17 open reading frame 67 | SEQ ID NO: 1710 |
| C17orf75 | Chromosome 17 open reading frame 75 | SEQ ID NOS: 1711-1719 |
| C17orf99 | Chromosome 17 open reading frame 99 | SEQ ID NOS: 1720-1722 |
| C18orf54 | Chromosome 18 open reading frame 54 | SEQ ID NOS: 1723-1727 |
| C19orf47 | Chromosome 19 open reading frame 47 | SEQ ID NOS: 1728-1735 |
| C19orf70 | Chromosome 19 open reading frame 70 | SEQ ID NOS: 1736-1739 |
| C1GALT1 | Core 1 synthase, glycoprotein-N- | SEQ ID NOS: 1740-1744 |
| | acetylgalactosamine 3-beta-galactosyltransferase 1 | |
| C1orf127 | Chromosome 1 open reading frame 127 | SEQ ID NOS: 1745-1748 |
| C1orf159 | Chromosome 1 open reading frame 159 | SEQ ID NOS: 1749-1761 |
| C1orf198 | Chromosome 1 open reading frame 198 | SEQ ID NOS: 1762-1766 |
| C1orf54 | Chromosome 1 open reading frame 54 | SEQ ID NOS: 1767-1769 |
| C1orf56 | Chromosome 1 open reading frame 56 | SEQ ID NO: 1770 |
| C1QA | Complement component 1, q subcomponent, A | SEQ ID NOS: 1771-1773 |
| | chain | |
| C1QB | Complement component 1, q subcomponent, B | SEQ ID NOS: 1774-1777 |
| | chain | |
| C1QC | Complement component 1, q subcomponent, C | SEQ ID NOS: 1778-1780 |
| | chain | |
| C1QL1 | Complement component 1, q subcomponent-like 1 | SEQ ID NO: 1781 |
| C1QL2 | Complement component 1, q subcomponent-like 2 | SEQ ID NO: 1782 |
| C1QL3 | Complement component 1, q subcomponent-like 3 | SEQ ID NOS: 1783-1784 |
| C1QL4 | Complement component 1, q subcomponent-like 4 | SEQ ID NO: 1785 |
| C1QTNF1 | C1q and tumor necrosis factor related protein 1 | SEQ ID NOS: 1786-1795 |
| FAM132A | Family with sequence similarity 132, member A | SEQ ID NO: 1796 |
| C1QTNF2 | C1q and tumor necrosis factor related protein 2 | SEQ ID NO: 1797 |
| C1QTNF3 | C1q and tumor necrosis factor related protein 3 | SEQ ID NOS: 1798-1799 |
| C1QTNF4 | C1q and tumor necrosis factor related protein 4 | SEQ ID NOS: 1800-1801 |
| C1QTNF5 | C1q and tumor necrosis factor related protein 5 | SEQ ID NOS: 1802-1804 |
| C1QTNF7 | C1q and tumor necrosis factor related protein 7 | SEQ ID NOS: 1805-1809 |
| C1QTNF8 | C1q and tumor necrosis factor related protein 8 | SEQ ID NOS: 1810-1811 |
| C1QTNF9 | C1q and tumor necrosis factor related protein 9 | SEQ ID NOS: 1812-1813 |
| C1QTNF9B | C1q and tumor necrosis factor related protein 9B | SEQ ID NOS: 1814-1816 |
| C1R | Complement component 1, r subcomponent | SEQ ID NOS: 1817-1825 |
| C1RL | Complement component 1, r subcomponent-like | SEQ ID NOS: 1826-1834 |
| C1S | Complement component 1, s subcomponent | SEQ ID NOS: 1835-1844 |
| C2 | Complement component 2 | SEQ ID NOS: 1845-1859 |
| C21orf33 | Chromosome 21 open reading frame 33 | SEQ ID NOS: 1860-1868 |
| C21orf62 | Chromosome 21 open reading frame 62 | SEQ ID NOS: 1869-1872 |
| C22orf15 | Chromosome 22 open reading frame 15 | SEQ ID NOS: 1873-1875 |
| C22orf46 | Chromosome 22 open reading frame 46 | SEQ ID NO: 1876 |
| C2CD2 | C2 calcium-dependent domain containing 2 | SEQ ID NOS: 1877-1879 |
| C2orf40 | Chromosome 2 open reading frame 40 | SEQ ID NOS: 1880-1882 |
| C2orf66 | Chromosome 2 open reading frame 66 | SEQ ID NO: 1883 |
| C2orf69 | Chromosome 2 open reading frame 69 | SEQ ID NO: 1884 |
| C2orf78 | Chromosome 2 open reading frame 78 | SEQ ID NO: 1885 |
| C3 | Complement component 3 | SEQ ID NOS: 1886-1890 |
| C3orf33 | Chromosome 3 open reading frame 33 | SEQ ID NOS: 1891-1895 |
| C3orf58 | Chromosome 3 open reading frame 58 | SEQ ID NOS: 1896-1899 |
| C4A | Complement component 4A (Rodgers blood group) | SEQ ID NOS: 1900-1901 |
| C4B | Complement component 4B (Chido blood group) | SEQ ID NOS: 1902-1903 |
| C4BPA | Complement component 4 binding protein, alpha | SEQ ID NOS: 1904-1906 |
| C4BPB | Complement component 4 binding protein, beta | SEQ ID NOS: 1907-1911 |
| C4orf48 | Chromosome 4 open reading frame 48 | SEQ ID NOS: 1912-1913 |
| C5 | Complement component 5 | SEQ ID NO: 1914 |
| C5orf46 | Chromosome 5 open reading frame 46 | SEQ ID NOS: 1915-1916 |
| C6 | Complement component 6 | SEQ ID NOS: 1917-1920 |
| C6orf120 | Chromosome 6 open reading frame 120 | SEQ ID NO: 1921 |
| C6orf15 | Chromosome 6 open reading frame 15 | SEQ ID NO: 1922 |
| C6orf58 | Chromosome 6 open reading frame 58 | SEQ ID NO: 1923 |
| C7 | Complement component 7 | SEQ ID NO: 1924 |
| C7orf57 | Chromosome 7 open reading frame 57 | SEQ ID NOS: 1925-1929 |
| C8A | Complement component 8, alpha polypeptide | SEQ ID NO: 1930 |
| C8B | Complement component 8, beta polypeptide | SEQ ID NOS: 1931-1933 |
| C8G | Complement component 8, gamma polypeptide | SEQ ID NOS: 1934-1935 |
| C9 | Complement component 9 | SEQ ID NO: 1936 |
| C9orf47 | Chromosome 9 open reading frame 47 | SEQ ID NOS: 1937-1939 |
| CA10 | Carbonic anhydrase X | SEQ ID NOS: 1940-1946 |
| CA11 | Carbonic anhydrase XI | SEQ ID NOS: 1947-1948 |
| CA6 | Carbonic anhydrase VI | SEQ ID NOS: 1949-1953 |
| CA9 | Carbonic anhydrase IX | SEQ ID NOS: 1954-1955 |
| CABLES1 | Cdk5 and Abl enzyme substrate 1 | SEQ ID NOS: 1956-1961 |
| CABP1 | Calcium binding protein 1 | SEQ ID NOS: 1962-1965 |
| CACNA2D1 | Calcium channel, voltage-dependent, alpha 2/delta | SEQ ID NOS: 1966-1969 |
| | subunit 1 | |
| CACNA2D4 | Calcium channel, voltage-dependent, alpha 2/delta | SEQ ID NOS: 1970-1983 |
| | subunit 4 | |
| CADM3 | Cell adhesion molecule 3 | SEQ ID NOS: 1984-1986 |
| CALCA | Calcitonin-related polypeptide alpha | SEQ ID NOS: 1987-1991 |
| CALCB | Calcitonin-related polypeptide beta | SEQ ID NOS: 1992-1994 |
| CALCR | Calcitonin receptor | SEQ ID NOS: 1995-2001 |
| CALCRL | Calcitonin receptor-like | SEQ ID NOS: 2002-2006 |
| FAM26D | Family with sequence similarity 26, member D | SEQ ID NOS: 2007-2011 |
| CALR | Calreticulin | SEQ ID NOS: 2012-2015 |
| CALR3 | Calreticulin 3 | SEQ ID NOS: 2016-2017 |
| CALU | Calumenin | SEQ ID NOS: 2018-2023 |
| CAMK2D | Calcium/calmodulin-dependent protein kinase II | SEQ ID NOS: 2024-2035 |
| | delta | |
| CAMP | Cathelicidin antimicrobial peptide | SEQ ID NO: 2036 |
| CANX | Calnexin | SEQ ID NOS: 2037-2051 |
| CARM1 | Coactivator-associated arginine methyltransferase 1 | SEQ ID NOS: 2052-2059 |
| CARNS1 | Carnosine synthase 1 | SEQ ID NOS: 2060-2062 |
| CARTPT | CART prepropeptide | SEQ ID NO: 2063 |
| CASQ1 | Calsequestrin 1 (fast-twitch, skeletal muscle) | SEQ ID NOS: 2064-2065 |
| CASQ2 | Calsequestrin 2 (cardiac muscle) | SEQ ID NO: 2066 |
| CATSPERG | Catsper channel auxiliary subunit gamma | SEQ ID NOS: 2067-2074 |
| CBLN1 | Cerebellin 1 precursor | SEQ ID NOS: 2075-2077 |
| CBLN2 | Cerebellin 2 precursor | SEQ ID NOS: 2078-2081 |
| CBLN3 | Cerebellin 3 precursor | SEQ ID NOS: 2082-2083 |
| CBLN4 | Cerebellin 4 precursor | SEQ ID NO: 2084 |
| CCBE1 | Collagen and calcium binding EGF domains 1 | SEQ ID NOS: 2085-2087 |
| CCDC112 | Coiled-coil domain containing 112 | SEQ ID NOS: 2088-2091 |
| CCDC129 | Coiled-coil domain containing 129 | SEQ ID NOS: 2092-2099 |
| CCDC134 | Coiled-coil domain containing 134 | SEQ ID NOS: 2100-2101 |
| CCDC149 | Coiled-coil domain containing 149 | SEQ ID NOS: 2102-2105 |
| CCDC3 | Coiled-coil domain containing 3 | SEQ ID NOS: 2106-2107 |
| CCDC80 | Coiled-coil domain containing 80 | SEQ ID NOS: 2108-2111 |
| CCDC85A | Coiled-coil domain containing 85A | SEQ ID NO: 2112 |
| CCDC88B | Coiled-coil domain containing 88B | SEQ ID NOS: 2113-2115 |
| CCER2 | Coiled-coil glutamate-rich protein 2 | SEQ ID NOS: 2116-2117 |
| CCK | Cholecystokinin | SEQ ID NOS: 2118-2120 |
| CCL1 | Chemokine (C-C motif) ligand 1 | SEQ ID NO: 2121 |
| CCL11 | Chemokine (C-C motif) ligand 11 | SEQ ID NO: 2122 |
| CCL13 | Chemokine (C-C motif) ligand 13 | SEQ ID NOS: 2123-2124 |
| CCL14 | Chemokine (C-C motif) ligand 14 | SEQ ID NOS: 2125-2128 |
| CCL15 | Chemokine (C-C motif) ligand 15 | SEQ ID NOS: 2129-2130 |
| CCL16 | Chemokine (C-C motif) ligand 16 | SEQ ID NOS: 2131-2133 |
| CCL17 | Chemokine (C-C motif) ligand 17 | SEQ ID NOS: 2134-2135 |
| CCL18 | Chemokine (C-C motif) ligand 18 (pulmonary and | SEQ ID NO: 2136 |
| | activation-regulated) | |
| CCL19 | Chemokine (C-C motif) ligand 19 | SEQ ID NOS: 2137-2138 |
| CCL2 | Chemokine (C-C motif) ligand 2 | SEQ ID NOS: 2139-2140 |
| CCL20 | Chemokine (C-C motif) ligand 20 | SEQ ID NOS: 2141-2143 |
| CCL21 | Chemokine (C-C motif) ligand 21 | SEQ ID NOS: 2144-2145 |
| CCL22 | Chemokine (C-C motif) ligand 22 | SEQ ID NO: 2146 |
| CCL23 | Chemokine (C-C motif) ligand 23 | SEQ ID NOS: 2147-2149 |
| CCL24 | Chemokine (C-C motif) ligand 24 | SEQ ID NOS: 2150-2151 |
| CCL25 | Chemokine (C-C motif) ligand 25 | SEQ ID NOS: 2152-2155 |
| CCL26 | Chemokine (C-C motif) ligand 26 | SEQ ID NOS: 2156-2157 |
| CCL27 | Chemokine (C-C motif) ligand 27 | SEQ ID NO: 2158 |
| CCL28 | Chemokine (C-C motif) ligand 28 | SEQ ID NOS: 2159-2161 |
| CCL3 | Chemokine (C-C motif) ligand 3 | SEQ ID NO: 2162 |
| CCL3L3 | Chemokine (C-C motif) ligand 3-like 3 | SEQ ID NO: 2163 |
| CCL4 | Chemokine (C-C motif) ligand 4 | SEQ ID NOS: 2164-2165 |
| CCL4L2 | Chemokine (C-C motif) ligand 4-like 2 | SEQ ID NOS: 2166-2175 |
| CCL5 | Chemokine (C-C motif) ligand 5 | SEQ ID NOS: 2176-2178 |
| CCL7 | Chemokine (C-C motif) ligand 7 | SEQ ID NOS: 2179-2181 |
| CCL8 | Chemokine (C-C motif) ligand 8 | SEQ ID NO: 2182 |
| CCNB1IP1 | Cyclin B1 interacting protein 1, E3 ubiquitin protein | SEQ ID NOS: 2183-2194 |
| | ligase | |
| CCNL1 | Cyclin L1 | SEQ ID NOS: 2195-2203 |
| CCNL2 | Cyclin L2 | SEQ ID NOS: 2204-2211 |
| CD14 | CD14 molecule | SEQ ID NOS: 2212-2216 |
| CD160 | CD160 molecule | SEQ ID NOS: 2217-2221 |
| CD164 | CD164 molecule, sialomucin | SEQ ID NOS: 2222-2227 |
| CD177 | CD177 molecule | SEQ ID NOS: 2228-2230 |
| CD1E | CD1e molecule | SEQ ID NOS: 2231-2244 |
| CD2 | CD2 molecule | SEQ ID NOS: 2245-2246 |
| CD200 | CD200 molecule | SEQ ID NOS: 2247-2253 |
| CD200R1 | CD200 receptor 1 | SEQ ID NOS: 2254-2258 |
| CD22 | CD22 molecule | SEQ ID NOS: 2259-2276 |
| CD226 | CD226 molecule | SEQ ID NOS: 2277-2284 |
| CD24 | CD24 molecule | SEQ ID NOS: 2285-2291 |
| CD276 | CD276 molecule | SEQ ID NOS: 2292-2307 |
| CD300A | CD300a molecule | SEQ ID NOS: 2308-2312 |
| CD300LB | CD300 molecule-like family member b | SEQ ID NOS: 2313-2314 |
| CD300LF | CD300 molecule-like family member f | SEQ ID NOS: 2315-2323 |
| CD300LG | CD300 molecule-like family member g | SEQ ID NOS: 2324-2329 |
| CD3D | CD3d molecule, delta (CD3-TCR complex) | SEQ ID NOS: 2330-2333 |
| CD4 | CD4 molecule | SEQ ID NOS: 2334-2336 |
| CD40 | CD40 molecule, TNF receptor superfamily member 5 | SEQ ID NOS: 2337-2340 |
| CD44 | CD44 molecule (Indian blood group) | SEQ ID NOS: 2341-2367 |
| CD48 | CD48 molecule | SEQ ID NOS: 2368-2370 |
| CD5 | CD5 molecule | SEQ ID NOS: 2371-2372 |
| CD55 | CD55 molecule, decay accelerating factor for | SEQ ID NOS: 2373-2383 |
| | complement (Cromer blood group) | |
| CD59 | CD59 molecule, complement regulatory protein | SEQ ID NOS: 2384-2394 |
| CD5L | CD5 molecule-like | SEQ ID NO: 2395 |
| CD6 | CD6 molecule | SEQ ID NOS: 2396-2403 |
| CD68 | CD68 molecule | SEQ ID NOS: 2404-2407 |
| CD7 | CD7 molecule | SEQ ID NOS: 2408-2413 |
| CD79A | CD79a molecule, immunoglobulin-associated alpha | SEQ ID NOS: 2414-2416 |
| CD80 | CD80 molecule | SEQ ID NOS: 2417-2419 |
| CD86 | CD86 molecule | SEQ ID NOS: 2420-2426 |
| CD8A | CD8a molecule | SEQ ID NOS: 2427-2430 |
| CD8B | CD8b molecule | SEQ ID NOS: 2431-2436 |
| CD99 | CD99 molecule | SEQ ID NOS: 2437-2445 |
| CDC23 | Cell division cycle 23 | SEQ ID NOS: 2446-2450 |
| CDC40 | Cell division cycle 40 | SEQ ID NOS: 2451-2453 |
| CDC45 | Cell division cycle 45 | SEQ ID NOS: 2454-2460 |
| CDCP1 | CUB domain containing protein 1 | SEQ ID NOS: 2461-2462 |
| CDCP2 | CUB domain containing protein 2 | SEQ ID NOS: 2463-2464 |
| CDH1 | Cadherin 1, type 1 | SEQ ID NOS: 2465-2472 |
| CDH11 | Cadherin 11, type 2, OB-cadherin (osteoblast) | SEQ ID NOS: 2473-2482 |
| CDH13 | Cadherin 13 | SEQ ID NOS: 2483-2492 |
| CDH17 | Cadherin 17, LI cadherin (liver-intestine) | SEQ ID NOS: 2493-2497 |
| CDH18 | Cadherin 18, type 2 | SEQ ID NOS: 2498-2504 |
| CDH19 | Cadherin 19, type 2 | SEQ ID NOS: 2505-2509 |
| CDH23 | Cadherin-related 23 | SEQ ID NOS: 2510-2525 |
| CDH5 | Cadherin 5, type 2 (vascular endothelium) | SEQ ID NOS: 2526-2533 |
| CDHR1 | Cadherin-related family member 1 | SEQ ID NOS: 2534-2539 |
| CDHR4 | Cadherin-related family member 4 | SEQ ID NOS: 2540-2544 |
| CDHR5 | Cadherin-related family member 5 | SEQ ID NOS: 2545-2551 |
| CDKN2A | Cyclin-dependent kinase inhibitor 2A | SEQ ID NOS: 2552-2562 |
| CDNF | Cerebral dopamine neurotrophic factor | SEQ ID NOS: 2563-2564 |
| CDON | Cell adhesion associated, oncogene regulated | SEQ ID NOS: 2565-2572 |
| CDSN | Corneodesmosin | SEQ ID NO: 2573 |
| CEACAM16 | Carcinoembryonic antigen-related cell adhesion | SEQ ID NOS: 2574-2575 |
| | molecule 16 | |
| CEACAM18 | Carcinoembryonic antigen-related cell adhesion | SEQ ID NO: 2576 |
| | molecule 18 | |
| CEACAM19 | Carcinoembryonic antigen-related cell adhesion | SEQ ID NOS: 2577-2583 |
| | molecule 19 | |
| CEACAM5 | Carcinoembryonic antigen-related cell adhesion | SEQ ID NOS: 2584-2591 |
| | molecule 5 | |
| CEACAM7 | Carcinoembryonic antigen-related cell adhesion | SEQ ID NOS: 2592-2594 |
| | molecule 7 | |
| CEACAM8 | Carcinoembryonic antigen-related cell adhesion | SEQ ID NOS: 2595-2596 |
| | molecule 8 | |
| CEL | Carboxyl ester lipase | SEQ ID NO: 2597 |
| CELA2A | Chymotrypsin-like elastase family, member 2A | SEQ ID NO: 2598 |
| CELA2B | Chymotrypsin-like elastase family, member 2B | SEQ ID NOS: 2599-2600 |
| CELA3A | Chymotrypsin-like elastase family, member 3A | SEQ ID NOS: 2601-2603 |
| CELA3B | Chymotrypsin-like elastase family, member 3B | SEQ ID NOS: 2604-2606 |
| CEMIP | Cell migration inducing protein, hyaluronan binding | SEQ ID NOS: 2607-2611 |
| CEP89 | Centrosomal protein 89 kDa | SEQ ID NOS: 2612-2617 |
| CER1 | Cerberus 1, DAN family BMP antagonist | SEQ ID NO: 2618 |
| CERCAM | Cerebral endothelial cell adhesion molecule | SEQ ID NOS: 2619-2626 |
| CERS1 | Ceramide synthase 1 | SEQ ID NOS: 2627-2631 |
| CES1 | Carboxylesterase 1 | SEQ ID NOS: 2632-2637 |
| CES3 | Carboxylesterase 3 | SEQ ID NOS: 2638-2642 |
| CES4A | Carboxylesterase 4A | SEQ ID NOS: 2643-2648 |
| CES5A | Carboxylesterase 5A | SEQ ID NOS: 2649-2656 |
| CETP | Cholesteryl ester transfer protein, plasma | SEQ ID NOS: 2657-2659 |
| CCDC108 | Coiled-coil domain containing 108 | SEQ ID NOS: 2660-2669 |
| CFB | Complement factor B | SEQ ID NOS: 2670-2674 |
| CFC1 | Cripto, FRL-1, cryptic family 1 | SEQ ID NOS: 2675-2677 |
| CFC1B | Cripto, FRL-1, cryptic family 1B | SEQ ID NOS: 2678-2680 |
| CFD | Complement factor D (adipsin) | SEQ ID NOS: 2681-2682 |
| CFDP1 | Craniofacial development protein 1 | SEQ ID NOS: 2683-2686 |
| CFH | Complement factor H | SEQ ID NOS: 2687-2689 |
| CFHR1 | Complement factor H-related 1 | SEQ ID NOS: 2690-2691 |
| CFHR2 | Complement factor H-related 2 | SEQ ID NOS: 2692-2693 |
| CFHR3 | Complement factor H-related 3 | SEQ ID NOS: 2694-2698 |
| CFHR4 | Complement factor H-related 4 | SEQ ID NOS: 2699-2702 |
| CFHR5 | Complement factor H-related 5 | SEQ ID NO: 2703 |
| CFI | Complement factor I | SEQ ID NOS: 2704-2708 |
| CFP | Complement factor properdin | SEQ ID NOS: 2709-2712 |
| CGA | Glycoprotein hormones, alpha polypeptide | SEQ ID NOS: 2713-2717 |
| CGB1 | Chorionic gonadotropin, beta polypeptide 1 | SEQ ID NOS: 2718-2719 |
| CGB2 | Chorionic gonadotropin, beta polypeptide 2 | SEQ ID NOS: 2720-2721 |
| CGB | Chorionic gonadotropin, beta polypeptide | SEQ ID NO: 2722 |
| CGB5 | Chorionic gonadotropin, beta polypeptide 5 | SEQ ID NO: 2723 |
| CGB7 | Chorionic gonadotropin, beta polypeptide 7 | SEQ ID NOS: 2724-2726 |
| CGB8 | Chorionic gonadotropin, beta polypeptide 8 | SEQ ID NO: 2727 |
| CGREF1 | Cell growth regulator with EF-hand domain 1 | SEQ ID NOS: 2728-2735 |
| CHAD | Chondroadherin | SEQ ID NOS: 2736-2738 |
| CHADL | Chondroadherin-like | SEQ ID NOS: 2739-2741 |
| CHEK2 | Checkpoint kinase 2 | SEQ ID NOS: 2742-2763 |
| CHGA | Chromogranin A | SEQ ID NOS: 2764-2766 |
| CHGB | Chromogranin B | SEQ ID NOS: 2767-2768 |
| CHI3L1 | Chitinase 3-like 1 (cartilage glycoprotein-39) | SEQ ID NOS: 2769-2770 |
| CHI3L2 | Chitinase 3-like 2 | SEQ ID NOS: 2771-2784 |
| CHIA | Chitinase, acidic | SEQ ID NOS: 2785-2793 |
| CHID1 | Chitinase domain containing 1 | SEQ ID NOS: 2794-2812 |
| CHIT1 | Chitinase 1 (chitotriosidase) | SEQ ID NOS: 2813-2816 |
| CHL1 | Cell adhesion molecule L1-like | SEQ ID NOS: 2817-2825 |
| CHN1 | Chimerin 1 | SEQ ID NOS: 2826-2836 |
| CHPF | Chondroitin polymerizing factor | SEQ ID NOS: 2837-2839 |
| CHPF2 | Chondroitin polymerizing factor 2 | SEQ ID NOS: 2840-2843 |
| CHRD | Chordin | SEQ ID NOS: 2844-2849 |
| CHRDL1 | Chordin-like 1 | SEQ ID NOS: 2850-2854 |
| CHRDL2 | Chordin-like 2 | SEQ ID NOS: 2855-2863 |
| CHRNA2 | Cholinergic receptor, nicotinic, alpha 2 (neuronal) | SEQ ID NOS: 2864-2872 |
| CHRNA5 | Cholinergic receptor, nicotinic, alpha 5 (neuronal) | SEQ ID NOS: 2873-2876 |
| CHRNB1 | Cholinergic receptor, nicotinic, beta 1 (muscle) | SEQ ID NOS: 2877-2882 |
| CHRND | Cholinergic receptor, nicotinic, delta (muscle) | SEQ ID NOS: 2883-2888 |
| CHST1 | Carbohydrate (keratan sulfate Gal-6) | SEQ ID NO: 2889 |
| | sulfotransferase 1 | |
| CHST10 | Carbohydrate sulfotransferase 10 | SEQ ID NOS: 2890-2897 |
| CHST11 | Carbohydrate (chondroitin 4) sulfotransferase 11 | SEQ ID NOS: 2898-2902 |
| CHST13 | Carbohydrate (chondroitin 4) sulfotransferase 13 | SEQ ID NOS: 2903-2904 |
| CHST4 | Carbohydrate (N-acetylglucosamine 6-O) | SEQ ID NOS: 2905-2906 |
| | sulfotransferase 4 | |
| CHST5 | Carbohydrate (N-acetylglucosamine 6-O) | SEQ ID NOS: 2907-2908 |
| | sulfotransferase 5 | |
| CHST6 | Carbohydrate (N-acetylglucosamine 6-O) | SEQ ID NOS: 2909-2910 |
| | sulfotransferase 6 | |
| CHST7 | Carbohydrate (N-acetylglucosamine 6-O) | SEQ ID NO: 2911 |
| | sulfotransferase 7 | |
| CHST8 | Carbohydrate (N-acetylgalactosamine 4-0) | SEQ ID NOS: 2912-2915 |
| | sulfotransferase 8 | |
| CHSY1 | Chondroitin sulfate synthase 1 | SEQ ID NOS: 2916-2917 |
| CHSY3 | Chondroitin sulfate synthase 3 | SEQ ID NO: 2918 |
| CHTF8 | Chromosome transmission fidelity factor 8 | SEQ ID NOS: 2919-2929 |
| CILP | Cartilage intermediate layer protein, nucleotide | SEQ ID NO: 2930 |
| | pyrophosphohydrolase | |
| CILP2 | Cartilage intermediate layer protein 2 | SEQ ID NOS: 2931-2932 |
| CKLF | Chemokine-like factor | SEQ ID NOS: 2933-2938 |
| CKMT1A | Creatine kinase, mitochondrial 1A | SEQ ID NOS: 2939-2944 |
| CKMT1B | Creatine kinase, mitochondrial 1B | SEQ ID NOS: 2945-2954 |
| CLCA1 | Chloride channel accessory 1 | SEQ ID NOS: 2955-2956 |
| CLCF1 | Cardiotrophin-like cytokine factor 1 | SEQ ID NOS: 2957-2958 |
| CLDN15 | Claudin 15 | SEQ ID NOS: 2959-2964 |
| CLDN7 | Claudin 7 | SEQ ID NOS: 2965-2971 |
| CLDND1 | Claudin domain containing 1 | SEQ ID NOS: 2972-2997 |
| CLEC11A | C-type lectin domain family 11, member A | SEQ ID NOS: 2998-3000 |
| CLEC16A | C-type lectin domain family 16, member A | SEQ ID NOS: 3001-3006 |
| CLEC18A | C-type lectin domain family 18, member A | SEQ ID NOS: 3007-3012 |
| CLEC18B | C-type lectin domain family 18, member B | SEQ ID NOS: 3013-3016 |
| CLEC18C | C-type lectin domain family 18, member C | SEQ ID NOS: 3017-3023 |
| CLEC19A | C-type lectin domain family 19, member A | SEQ ID NOS: 3024-3027 |
| CLEC2B | C-type lectin domain family 2, member B | SEQ ID NOS: 3028-3029 |
| CLEC3A | C-type lectin domain family 3, member A | SEQ ID NOS: 3030-3031 |
| CLEC3B | C-type lectin domain family 3, member B | SEQ ID NOS: 3032-3033 |
| CLGN | Calmegin | SEQ ID NOS: 3034-3036 |
| CLN5 | Ceroid-lipofuscinosis, neuronal 5 | SEQ ID NOS: 3037-3048 |
| CLPS | Colipase, pancreatic | SEQ ID NOS: 3049-3051 |
| CLPSL1 | Colipase-like 1 | SEQ ID NOS: 3052-3053 |
| CLPSL2 | Colipase-like 2 | SEQ ID NOS: 3054-3055 |
| CLPX | Caseinolytic mitochondrial matrix peptidase | SEQ ID NOS: 3056-3058 |
| | chaperone subunit | |
| CLSTN3 | Calsyntenin 3 | SEQ ID NOS: 3059-3065 |
| CLU | Clusterin | SEQ ID NOS: 3066-3079 |
| CLUL1 | Clusterin-like 1 (retinal) | SEQ ID NOS: 3080-3087 |
| CMA1 | Chymase 1, mast cell | SEQ ID NOS: 3088-3089 |
| CMPK1 | Cytidine monophosphate (UMP-CMP) kinase 1, | SEQ ID NOS: 3090-3093 |
| | cytosolic | |
| CNBD1 | Cyclic nucleotide binding domain containing 1 | SEQ ID NOS: 3094-3097 |
| CNDP1 | Carnosine dipeptidase 1 (metallopeptidase M20 | SEQ ID NOS: 3098-3100 |
| | family) | |
| RQCD1 | RCD1 required for cell differentiation1 homolog (S. | SEQ ID NOS: 3101-3107 |
| | pombe) | |
| CNPY2 | Canopy FGF signaling regulator 2 | SEQ ID NOS: 3108-3112 |
| CNPY3 | Canopy FGF signaling regulator 3 | SEQ ID NOS: 3113-3114 |
| CNPY4 | Canopy FGF signaling regulator 4 | SEQ ID NOS: 3115-3117 |
| CNTFR | Ciliary neurotrophic factor receptor | SEQ ID NOS: 3118-3121 |
| CNTN1 | Contactin 1 | SEQ ID NOS: 3122-3131 |
| CNTN2 | Contactin 2 (axonal) | SEQ ID NOS: 3132-3143 |
| CNTN3 | Contactin 3 (plasmacytoma associated) | SEQ ID NO: 3144 |
| CNTN4 | Contactin 4 | SEQ ID NOS: 3145-3153 |
| CNTN5 | Contactin 5 | SEQ ID NOS: 3154-3159 |
| CNTNAP2 | Contactin associated protein-like 2 | SEQ ID NOS: 3160-3163 |
| CNTNAP3 | Contactin associated protein-like 3 | SEQ ID NOS: 3164-3168 |
| CNTNAP3B | Contactin associated protein-like 3B | SEQ ID NOS: 3169-3177 |
| COASY | CoA synthase | SEQ ID NOS: 3178-3187 |
| COCH | Cochlin | SEQ ID NOS: 3188-3199 |
| COG3 | Component of oligomeric golgi complex 3 | SEQ ID NOS: 3200-3203 |
| COL10A1 | Collagen, type X, alpha 1 | SEQ ID NOS: 3204-3207 |
| COL11A1 | Collagen, type XI, alpha 1 | SEQ ID NOS: 3208-3218 |
| COL11A2 | Collagen, type XI, alpha 2 | SEQ ID NOS: 3219-3223 |
| COL12A1 | Collagen, type XII, alpha 1 | SEQ ID NOS: 3224-3231 |
| COL14A1 | Collagen, type XIV, alpha 1 | SEQ ID NOS: 3232-3239 |
| COL15A1 | Collagen, type XV, alpha 1 | SEQ ID NOS: 3240-3241 |
| COL16A1 | Collagen, type XVI, alpha 1 | SEQ ID NOS: 3242-3246 |
| COL18A1 | Collagen, type XVIII, alpha 1 | SEQ ID NOS: 3247-3251 |
| COL19A1 | Collagen, type XIX, alpha 1 | SEQ ID NOS: 3252-3254 |
| COL1A1 | Collagen, type I, alpha 1 | SEQ ID NOS: 3255-3256 |
| COL1A2 | Collagen, type I, alpha 2 | SEQ ID NOS: 3257-3258 |
| COL20A1 | Collagen, type XX, alpha 1 | SEQ ID NOS: 3259-3262 |
| COL21A1 | Collagen, type XXI, alpha 1 | SEQ ID NOS: 3263-3268 |
| COL22A1 | Collagen, type XXII, alpha 1 | SEQ ID NOS: 3269-3271 |
| COL24A1 | Collagen, type XXIV, alpha 1 | SEQ ID NOS: 3272-3275 |
| COL26A1 | Collagen, type XXVI, alpha 1 | SEQ ID NOS: 3276-3277 |
| COL27A1 | Collagen, type XXVII, alpha 1 | SEQ ID NOS: 3278-3280 |
| COL28A1 | Collagen, type XXVIII, alpha 1 | SEQ ID NOS: 3281-3285 |
| COL2A1 | Collagen, type II, alpha 1 | SEQ ID NOS: 3286-3287 |
| COL3A1 | Collagen, type III, alpha 1 | SEQ ID NOS: 3288-3290 |
| COL4A1 | Collagen, type IV, alpha 1 | SEQ ID NOS: 3291-3293 |
| COL4A2 | Collagen, type IV, alpha 2 | SEQ ID NOS: 3294-3296 |
| COL4A3 | Collagen, type IV, alpha 3 (Goodpasture antigen) | SEQ ID NOS: 3297-3300 |
| COL4A4 | Collagen, type IV, alpha 4 | SEQ ID NOS: 3301-3302 |
| COL4A5 | Collagen, type IV, alpha 5 | SEQ ID NOS: 3303-3309 |
| COL4A6 | Collagen, type IV, alpha 6 | SEQ ID NOS: 3310-3315 |
| COL5A1 | Collagen, type V, alpha 1 | SEQ ID NOS: 3316-3318 |
| COL5A2 | Collagen, type V, alpha 2 | SEQ ID NOS: 3319-3320 |
| COL5A3 | Collagen, type V, alpha 3 | SEQ ID NO: 3321 |
| COL6A1 | Collagen, type VI, alpha 1 | SEQ ID NOS: 3322-3323 |
| COL6A2 | Collagen, type VI, alpha 2 | SEQ ID NOS: 3324-3329 |
| COL6A3 | Collagen, type VI, alpha 3 | SEQ ID NOS: 3330-3338 |
| COL6A5 | Collagen, type VI, alpha 5 | SEQ ID NOS: 3339-3343 |
| COL6A6 | Collagen, type VI, alpha 6 | SEQ ID NOS: 3344-3346 |
| COL7A1 | Collagen, type VII, alpha 1 | SEQ ID NOS: 3347-3348 |
| COL8A1 | Collagen, type VIII, alpha 1 | SEQ ID NOS: 3349-3352 |
| COL8A2 | Collagen, type VIII, alpha 2 | SEQ ID NOS: 3353-3355 |
| COL9A1 | Collagen, type IX, alpha 1 | SEQ ID NOS: 3356-3359 |
| COL9A2 | Collagen, type IX, alpha 2 | SEQ ID NOS: 3360-3363 |
| COL9A3 | Collagen, type IX, alpha 3 | SEQ ID NOS: 3364-3365 |
| COLEC10 | Collectin sub-family member 10 (C-type lectin) | SEQ ID NO: 3366 |
| COLEC11 | Collectin sub-family member 11 | SEQ ID NOS: 3367-3376 |
| COLGALT1 | Collagen beta(1-O)galactosyltransferase 1 | SEQ ID NOS: 3377-3379 |
| COLGALT2 | Collagen beta(1-O)galactosyltransferase 2 | SEQ ID NOS: 3380-3382 |
| COLQ | Collagen-like tail subunit (single strand of | SEQ ID NOS: 3383-3387 |
| | homotrimer) of asymmetric acetylcholinesterase | |
| COMP | Cartilage oligomeric matrix protein | SEQ ID NOS: 3388-3390 |
| COPS6 | COP9 signalosome subunit 6 | SEQ ID NOS: 3391-3394 |
| COQ6 | Coenzyme Q6 monooxygenase | SEQ ID NOS: 3395-3402 |
| CORT | Cortistatin | SEQ ID NO: 3403 |
| CP | Ceruloplasmin (ferroxidase) | SEQ ID NOS: 3404-3408 |
| CPA1 | Carboxypeptidase A1 (pancreatic) | SEQ ID NOS: 3409-3413 |
| CPA2 | Carboxypeptidase A2 (pancreatic) | SEQ ID NOS: 3414-3415 |
| CPA3 | Carboxypeptidase A3 (mast cell) | SEQ ID NO: 3416 |
| CPA4 | Carboxypeptidase A4 | SEQ ID NOS: 3417-3422 |
| CPA6 | Carboxypeptidase A6 | SEQ ID NOS: 3423-3425 |
| CPAMD8 | C3 and PZP-like, alpha-2-macroglobulin domain | SEQ ID NOS: 3426-3431 |
| | containing 8 | |
| CPB1 | Carboxypeptidase B1 (tissue) | SEQ ID NOS: 3432-3436 |
| CPB2 | Carboxypeptidase B2 (plasma) | SEQ ID NOS: 3437-3439 |
| CPE | Carboxypeptidase E | SEQ ID NOS: 3440-3444 |
| CPM | Carboxypeptidase M | SEQ ID NOS: 3445-3454 |
| CPN1 | Carboxypeptidase N, polypeptide 1 | SEQ ID NOS: 3455-3456 |
| CPN2 | Carboxypeptidase N, polypeptide 2 | SEQ ID NOS: 3457-3458 |
| CPO | Carboxypeptidase O | SEQ ID NO: 3459 |
| CPQ | Carboxypeptidase Q | SEQ ID NOS: 3460-3465 |
| CPVL | Carboxypeptidase, vitellogenic-like | SEQ ID NOS: 3466-3476 |
| CPXM1 | Carboxypeptidase X (M14 family), member 1 | SEQ ID NO: 3477 |
| CPXM2 | Carboxypeptidase X (M14 family), member 2 | SEQ ID NOS: 3478-3479 |
| CPZ | Carboxypeptidase Z | SEQ ID NOS: 3480-3483 |
| CR1L | Complement component (3b/4b) receptor 1-like | SEQ ID NOS: 3484-3485 |
| CRB2 | Crumbs family member 2 | SEQ ID NOS: 3486-3488 |
| CREG1 | Cellular repressor of E1A-stimulated genes 1 | SEQ ID NO: 3489 |
| CREG2 | Cellular repressor of E1A-stimulated genes 2 | SEQ ID NO: 3490 |
| CRELD1 | Cysteine-rich with EGF-like domains 1 | SEQ ID NOS: 3491-3496 |
| CRELD2 | Cysteine-rich with EGF-like domains 2 | SEQ ID NOS: 3497-3501 |
| CRH | Corticotropin releasing hormone | SEQ ID NO: 3502 |
| CRHBP | Corticotropin releasing hormone binding protein | SEQ ID NOS: 3503-3504 |
| CRHR1 | Corticotropin releasing hormone receptor 1 | SEQ ID NOS: 3505-3516 |
| CRHR2 | Corticotropin releasing hormone receptor 2 | SEQ ID NOS: 3517-3523 |
| CRISP1 | Cysteine-rich secretory protein 1 | SEQ ID NOS: 3524-3527 |
| CRISP2 | Cysteine-rich secretory protein 2 | SEQ ID NOS: 3528-3530 |
| CRISP3 | Cysteine-rich secretory protein 3 | SEQ ID NOS: 3531-3534 |
| CRISPLD2 | Cysteine-rich secretory protein LCCL domain | SEQ ID NOS: 3535-3542 |
| | containing 2 | |
| CRLF1 | Cytokine receptor-like factor 1 | SEQ ID NOS: 3543-3544 |
| CRP | C-reactive protein, pentraxin-related | SEQ ID NOS: 3545-3549 |
| CRTAC1 | Cartilage acidic protein 1 | SEQ ID NOS: 3550-3554 |
| CRTAP | Cartilage associated protein | SEQ ID NOS: 3555-3556 |
| CRY2 | Cryptochrome circadian clock 2 | SEQ ID NOS: 3557-3560 |
| CSAD | Cysteine sulfinic acid decarboxylase | SEQ ID NOS: 3561-3573 |
| CSF1 | Colony stimulating factor 1 (macrophage) | SEQ ID NOS: 3574-3581 |
| CSF1R | Colony stimulating factor 1 receptor | SEQ ID NOS: 3582-3586 |
| CSF2 | Colony stimulating factor 2 (granulocyte- | SEQ ID NO: 3587 |
| | macrophage) | |
| CSF2RA | Colony stimulating factor 2 receptor, alpha, low- | SEQ ID NOS: 3588-3599 |
| | affinity (granulocyte-macrophage) | |
| CSF3 | Colony stimulating factor 3 (granulocyte) | SEQ ID NOS: 3600-3606 |
| CSGALNACT | Chondroitin sulfate N- | SEQ ID NOS: 3607-3615 |
| 1 | acetylgalactosaminyltransferase 1 | |
| CSH1 | Chorionic somatomammotropin hormone 1 | SEQ ID NOS: 3616-3619 |
| | (placental lactogen) | |
| CSH2 | Chorionic somatomammotropin hormone 2 | SEQ ID NOS: 3620-3624 |
| CSHL1 | Chorionic somatomammotropin hormone-like 1 | SEQ ID NOS: 3625-3631 |
| CSN1S1 | Casein alpha s1 | SEQ ID NOS: 3632-3637 |
| CSN2 | Casein beta | SEQ ID NO: 3638 |
| CSN3 | Casein kappa | SEQ ID NO: 3639 |
| CST1 | Cystatin SN | SEQ ID NOS: 3640-3641 |
| CST11 | Cystatin 11 | SEQ ID NOS: 3642-3643 |
| CST2 | Cystatin SA | SEQ ID NO: 3644 |
| CST3 | Cystatin C | SEQ ID NOS: 3645-3647 |
| CST4 | Cystatin S | SEQ ID NO: 3648 |
| CST5 | Cystatin D | SEQ ID NO: 3649 |
| CST6 | Cystatin E/M | SEQ ID NO: 3650 |
| CST7 | Cystatin F (leukocystatin) | SEQ ID NO: 3651 |
| CST8 | Cystatin 8 (cystatin-related epididymal specific) | SEQ ID NOS: 3652-3653 |
| CST9 | Cystatin 9 (testatin) | SEQ ID NO: 3654 |
| CST9L | Cystatin 9-like | SEQ ID NO: 3655 |
| CSTL1 | Cystatin-like 1 | SEQ ID NOS: 3656-3658 |
| CT55 | Cancer/testis antigen 55 | SEQ ID NOS: 3659-3660 |
| CTBS | Chitobiase, di-N-acetyl- | SEQ ID NOS: 3661-3663 |
| CTGF | Connective tissue growth factor | SEQ ID NO: 3664 |
| CTHRC1 | Collagen triple helix repeat containing 1 | SEQ ID NOS: 3665-3668 |
| CTLA4 | Cytotoxic T-lymphocyte-associated protein 4 | SEQ ID NOS: 3669-3672 |
| CTNS | Cystinosin, lysosomal cystine transporter | SEQ ID NOS: 3673-3680 |
| CTRB1 | Chymotrypsinogen B1 | SEQ ID NOS: 3681-3683 |
| CTRB2 | Chymotrypsinogen B2 | SEQ ID NOS: 3684-3687 |
| CTRC | Chymotrypsin C (caldecrin) | SEQ ID NOS: 3688-3689 |
| CTRL | Chymotrypsin-like | SEQ ID NOS: 3690-3692 |
| CTSA | Cathepsin A | SEQ ID NOS: 3693-3701 |
| CTSB | Cathepsin B | SEQ ID NOS: 3702-3726 |
| CTSC | Cathepsin C | SEQ ID NOS: 3727-3731 |
| CTSD | Cathepsin D | SEQ ID NOS: 3732-3742 |
| CTSE | Cathepsin E | SEQ ID NOS: 3743-3744 |
| CTSF | Cathepsin F | SEQ ID NOS: 3745-3748 |
| CTSG | Cathepsin G | SEQ ID NO: 3749 |
| CTSH | Cathepsin H | SEQ ID NOS: 3750-3755 |
| CTSK | Cathepsin K | SEQ ID NOS: 3756-3757 |
| CTSL | Cathepsin L | SEQ ID NOS: 3758-3760 |
| CTSO | Cathepsin O | SEQ ID NO: 3761 |
| CTSS | Cathepsin S | SEQ ID NOS: 3762-3766 |
| CTSV | Cathepsin V | SEQ ID NOS: 3767-3768 |
| CTSW | Cathepsin W | SEQ ID NOS: 3769-3771 |
| CTSZ | Cathepsin Z | SEQ ID NO: 3772 |
| CUBN | Cubilin (intrinsic factor-cobalamin receptor) | SEQ ID NOS: 3773-3776 |
| CUTA | CutA divalent cation tolerance homolog (E. coli) | SEQ ID NOS: 3777-3786 |
| CX3CL1 | Chemokine (C-X3-C motif) ligand 1 | SEQ ID NOS: 3787-3790 |
| CXADR | Coxsackie virus and adenovirus receptor | SEQ ID NOS: 3791-3795 |
| CXCL1 | Chemokine (C-X-C motif) ligand 1 (melanoma growth | SEQ ID NO: 3796 |
| | stimulating activity, alpha) | |
| CXCL10 | Chemokine (C-X-C motif) ligand 10 | SEQ ID NO: 3797 |
| CXCL11 | Chemokine (C-X-C motif) ligand 11 | SEQ ID NOS: 3798-3799 |
| CXCL12 | Chemokine (C-X-C motif) ligand 12 | SEQ ID NOS: 3800-3805 |
| CXCL13 | Chemokine (C-X-C motif) ligand 13 | SEQ ID NO: 3806 |
| CXCL14 | Chemokine (C-X-C motif) ligand 14 | SEQ ID NOS: 3807-3808 |
| CXCL17 | Chemokine (C-X-C motif) ligand 17 | SEQ ID NOS: 3809-3810 |
| CXCL2 | Chemokine (C-X-C motif) ligand 2 | SEQ ID NO: 3811 |
| CXCL3 | Chemokine (C-X-C motif) ligand 3 | SEQ ID NO: 3812 |
| CXCL5 | Chemokine (C-X-C motif) ligand 5 | SEQ ID NO: 3813 |
| CXCL6 | Chemokine (C-X-C motif) ligand 6 | SEQ ID NOS: 3814-3815 |
| CXCL8 | Chemokine (C-X-C motif) ligand 8 | SEQ ID NOS: 3816-3817 |
| CXCL9 | Chemokine (C-X-C motif) ligand 9 | SEQ ID NO: 3818 |
| CXorf36 | Chromosome X open reading frame 36 | SEQ ID NOS: 3819-3820 |
| CYB5D2 | Cytochrome b5 domain containing 2 | SEQ ID NOS: 3821-3824 |
| CYHR1 | Cysteine/histidine-rich 1 | SEQ ID NOS: 3825-3832 |
| CYP17A1 | Cytochrome P450, family 17, subfamily A, | SEQ ID NOS: 3833-3837 |
| | polypeptide 1 | |
| CYP20A1 | Cytochrome P450, family 20, subfamily A, | SEQ ID NOS: 3838-3844 |
| | polypeptide 1 | |
| CYP21A2 | Cytochrome P450, family 21, subfamily A, | SEQ ID NOS: 3845-3852 |
| | polypeptide 2 | |
| CYP26B1 | Cytochrome P450, family 26, subfamily B, | SEQ ID NOS: 3853-3857 |
| | polypeptide 1 | |
| CYP2A6 | Cytochrome P450, family 2, subfamily A, | SEQ ID NOS: 3858-3859 |
| | polypeptide 6 | |
| CYP2A7 | Cytochrome P450, family 2, subfamily A, | SEQ ID NOS: 3860-3862 |
| | polypeptide 7 | |
| CYP2B6 | Cytochrome P450, family 2, subfamily B, | SEQ ID NOS: 3863-3866 |
| | polypeptide 6 | |
| CYP2C18 | Cytochrome P450, family 2, subfamily C, | SEQ ID NOS: 3867-3868 |
| | polypeptide 18 | |
| CYP2C19 | Cytochrome P450, family 2, subfamily C, | SEQ ID NOS: 3869-3870 |
| | polypeptide 19 | |
| CYP2C8 | Cytochrome P450, family 2, subfamily C, | SEQ ID NOS: 3871-3878 |
| | polypeptide 8 | |
| CYP2C9 | Cytochrome P450, family 2, subfamily C, | SEQ ID NOS: 3879-3881 |
| | polypeptide 9 | |
| CYP2E1 | Cytochrome P450, family 2, subfamily E, | SEQ ID NOS: 3882-3887 |
| | polypeptide 1 | |
| CYP2F1 | Cytochrome P450, family 2, subfamily F, | SEQ ID NOS: 3888-3891 |
| | polypeptide 1 | |
| CYP2J2 | Cytochrome P450, family 2, subfamily J, | SEQ ID NO: 3892 |
| | polypeptide 2 | |
| CYP2R1 | Cytochrome P450, family 2, subfamily R, | SEQ ID NOS: 3893-3898 |
| | polypeptide 1 | |
| CYP2S1 | Cytochrome P450, family 2, subfamily S, | SEQ ID NOS: 3899-3904 |
| | polypeptide 1 | |
| CYP2W1 | Cytochrome P450, family 2, subfamily W, | SEQ ID NOS: 3905-3907 |
| | polypeptide 1 | |
| CYP46A1 | Cytochrome P450, family 46, subfamily A, | SEQ ID NOS: 3908-3912 |
| | polypeptide 1 | |
| CYP4F11 | Cytochrome P450, family 4, subfamily F, | SEQ ID NOS: 3913-3917 |
| | polypeptide 11 | |
| CYP4F2 | Cytochrome P450, family 4, subfamily F, | SEQ ID NOS: 3918-3922 |
| | polypeptide 2 | |
| CYR61 | Cysteine-rich, angiogenic inducer, 61 | SEQ ID NO: 3923 |
| CYTL1 | Cytokine-like 1 | SEQ ID NOS: 3924-3926 |
| D2HGDH | D-2-hydroxyglutarate dehydrogenase | SEQ ID NOS: 3927-3935 |
| DAG1 | Dystroglycan 1 (dystrophin-associated glycoprotein | SEQ ID NOS: 3936-3950 |
| | 1) | |
| DAND5 | DAN domain family member 5, BMP antagonist | SEQ ID NOS: 3951-3952 |
| DAO | D-amino-acid oxidase | SEQ ID NOS: 3953-3958 |
| DAZAP2 | DAZ associated protein 2 | SEQ ID NOS: 3959-3967 |
| DBH | Dopamine beta-hydroxylase (dopamine beta- | SEQ ID NOS: 3968-3969 |
| | monooxygenase) | |
| DBNL | Drebrin-like | SEQ ID NOS: 3970-3987 |
| DCD | Dermcidin | SEQ ID NOS: 3988-3990 |
| DCN | Decorin | SEQ ID NOS: 3991-4009 |
| DDIAS | DNA damage-induced apoptosis suppressor | SEQ ID NOS: 4010-4019 |
| DDOST | Dolichyl-diphosphooligosaccharide-protein | SEQ ID NOS: 4020-4023 |
| | glycosyltransferase subunit (non-catalytic) | |
| DDR1 | Discoidin domain receptor tyrosine kinase 1 | SEQ ID NOS: 4024-4069 |
| DDR2 | Discoidin domain receptor tyrosine kinase 2 | SEQ ID NOS: 4070-4075 |
| DDT | D-dopachrome tautomerase | SEQ ID NOS: 4076-4081 |
| DDX17 | DEAD (Asp-Glu-Ala-Asp) box helicase 17 | SEQ ID NOS: 4082-4086 |
| DDX20 | DEAD (Asp-Glu-Ala-Asp) box polypeptide 20 | SEQ ID NOS: 4087-4089 |
| DDX25 | DEAD (Asp-Glu-Ala-Asp) box helicase 25 | SEQ ID NOS: 4090-4096 |
| DDX28 | DEAD (Asp-Glu-Ala-Asp) box polypeptide 28 | SEQ ID NO: 4097 |
| DEAF1 | DEAF1 transcription factor | SEQ ID NOS: 4098-4100 |
| DEF8 | Differentially expressed in FDCP 8 homolog (mouse) | SEQ ID NOS: 4101-4120 |
| DEFA1 | Defensin, alpha 1 | SEQ ID NOS: 4121-4122 |
| DEFA1B | Defensin, alpha 1B | SEQ ID NO: 4123 |
| DEFA3 | Defensin, alpha 3, neutrophil-specific | SEQ ID NO: 4124 |
| DEFA4 | Defensin, alpha 4, corticostatin | SEQ ID NO: 4125 |
| DEFA5 | Defensin, alpha 5, Paneth cell-specific | SEQ ID NO: 4126 |
| DEFA6 | Defensin, alpha 6, Paneth cell-specific | SEQ ID NO: 4127 |
| DEFB1 | Defensin, beta 1 | SEQ ID NO: 4128 |
| DEFB103A | Defensin, beta 103A | SEQ ID NO: 4129 |
| DEFB103B | Defensin, beta 103B | SEQ ID NO: 4130 |
| DEFB104A | Defensin, beta 104A | SEQ ID NO: 4131 |
| DEFB104B | Defensin, beta 104B | SEQ ID NO: 4132 |
| DEFB105A | Defensin, beta 105A | SEQ ID NO: 4133 |
| DEFB105B | Defensin, beta 105B | SEQ ID NO: 4134 |
| DEFB106A | Defensin, beta 106A | SEQ ID NO: 4135 |
| DEFB106B | Defensin, beta 106B | SEQ ID NO: 4136 |
| DEFB107A | Defensin, beta 107A | SEQ ID NO: 4137 |
| DEFB107B | Defensin, beta 107B | SEQ ID NO: 4138 |
| DEFB108B | Defensin, beta 108B | SEQ ID NO: 4139 |
| DEFB110 | Defensin, beta 110 | SEQ ID NOS: 4140-4141 |
| DEFB113 | Defensin, beta 113 | SEQ ID NO: 4142 |
| DEFB114 | Defensin, beta 114 | SEQ ID NO: 4143 |
| DEFB115 | Defensin, beta 115 | SEQ ID NO: 4144 |
| DEFB116 | Defensin, beta 116 | SEQ ID NO: 4145 |
| DEFB118 | Defensin, beta 118 | SEQ ID NO: 4146 |
| DEFB119 | Defensin, beta 119 | SEQ ID NOS: 4147-4149 |
| DEFB121 | Defensin, beta 121 | SEQ ID NO: 4150 |
| DEFB123 | Defensin, beta 123 | SEQ ID NO: 4151 |
| DEFB124 | Defensin, beta 124 | SEQ ID NO: 4152 |
| DEFB125 | Defensin, beta 125 | SEQ ID NO: 4153 |
| DEFB126 | Defensin, beta 126 | SEQ ID NO: 4154 |
| DEFB127 | Defensin, beta 127 | SEQ ID NO: 4155 |
| DEFB128 | Defensin, beta 128 | SEQ ID NO: 4156 |
| DEFB129 | Defensin, beta 129 | SEQ ID NO: 4157 |
| DEFB130 | Defensin, beta 130 | SEQ ID NO: 4158 |
| RP11- | | SEQ ID NO: 4159 |
| 1236K1.1 | | |
| DEFB131 | Defensin, beta 131 | SEQ ID NO: 4160 |
| CTD- | | SEQ ID NO: 4161 |
| 2313N18.7 | | |
| DEFB132 | Defensin, beta 132 | SEQ ID NO: 4162 |
| DEFB133 | Defensin, beta 133 | SEQ ID NO: 4163 |
| DEFB134 | Defensin, beta 134 | SEQ ID NOS: 4164-4165 |
| DEFB135 | Defensin, beta 135 | SEQ ID NO: 4166 |
| DEFB136 | Defensin, beta 136 | SEQ ID NO: 4167 |
| DEFB4A | Defensin, beta 4A | SEQ ID NO: 4168 |
| DEFB4B | Defensin, beta 4B | SEQ ID NO: 4169 |
| C10orf10 | Chromosome 10 open reading frame 10 | SEQ ID NOS: 4170-4171 |
| DGCR2 | DiGeorge syndrome critical region gene 2 | SEQ ID NOS: 4172-4175 |
| DHH | Desert hedgehog | SEQ ID NO: 4176 |
| DHRS4 | Dehydrogenase/reductase (SDR family) member 4 | SEQ ID NOS: 4177-4184 |
| DHRS4L2 | Dehydrogenase/reductase (SDR family) member 4 | SEQ ID NOS: 4185-4194 |
| | like 2 | |
| DHRS7 | Dehydrogenase/reductase (SDR family) member 7 | SEQ ID NOS: 4195-4202 |
| DHRS7C | Dehydrogenase/reductase (SDR family) member 7C | SEQ ID NOS: 4203-4205 |
| DHRS9 | Dehydrogenase/reductase (SDR family) member 9 | SEQ ID NOS: 4206-4213 |
| DHRSX | Dehydrogenase/reductase (SDR family) X-linked | SEQ ID NOS: 4214-4218 |
| DHX29 | DEAH (Asp-Glu-Ala-His) box polypeptide 29 | SEQ ID NOS: 4219-4221 |
| DHX30 | DEAH (Asp-Glu-Ala-His) box helicase 30 | SEQ ID NOS: 4222-4229 |
| DHX8 | DEAH (Asp-Glu-Ala-His) box polypeptide 8 | SEQ ID NOS: 4230-4234 |
| DIO2 | Deiodinase, iodothyronine, type II | SEQ ID NOS: 4235-4244 |
| DIXDC1 | DIX domain containing 1 | SEQ ID NOS: 4245-4248 |
| DKK1 | Dickkopf WNT signaling pathway inhibitor 1 | SEQ ID NO: 4249 |
| DKK2 | Dickkopf WNT signaling pathway inhibitor 2 | SEQ ID NOS: 4250-4252 |
| DKK3 | Dickkopf WNT signaling pathway inhibitor 3 | SEQ ID NOS: 4253-4258 |
| DKK4 | Dickkopf WNT signaling pathway inhibitor 4 | SEQ ID NO: 4259 |
| DKKL1 | Dickkopf-like 1 | SEQ ID NOS: 4260-4265 |
| DLG4 | Discs, large homolog 4 (Drosophila) | SEQ ID NOS: 4266-4274 |
| DLK1 | Delta-like 1 homolog (Drosophila) | SEQ ID NOS: 4275-4278 |
| DLL1 | Delta-like 1 (Drosophila) | SEQ ID NOS: 4279-4280 |
| DLL3 | Delta-like 3 (Drosophila) | SEQ ID NOS: 4281-4283 |
| DMBT1 | Deleted in malignant brain tumors 1 | SEQ ID NOS: 4284-4290 |
| DMKN | Dermokine | SEQ ID NOS: 4291-4337 |
| DMP1 | Dentin matrix acidic phosphoprotein 1 | SEQ ID NOS: 4338-4339 |
| DMRTA2 | DMRT-like family A2 | SEQ ID NOS: 4340-4341 |
| DNAAF5 | Dynein, axonemal, assembly factor 5 | SEQ ID NOS: 4342-4345 |
| DNAH14 | Dynein, axonemal, heavy chain 14 | SEQ ID NOS: 4346-4360 |
| DNAJB11 | DnaJ (Hsp40) homolog, subfamily B, member 11 | SEQ ID NOS: 4361-4362 |
| DNAJB9 | DnaJ (Hsp40) homolog, subfamily B, member 9 | SEQ ID NO: 4363 |
| DNAJC25- | DNAJC25-GNG10 readthrough | SEQ ID NO: 4364 |
| GNG10 | | |
| DNAJC3 | DnaJ (Hsp40) homolog, subfamily C, member 3 | SEQ ID NOS: 4365-4366 |
| DNASE1 | Deoxyribonuclease I | SEQ ID NOS: 4367-4377 |
| DNASE1L1 | Deoxyribonuclease I-like 1 | SEQ ID NOS: 4378-4388 |
| DNASE1L2 | Deoxyribonuclease I-like 2 | SEQ ID NOS: 4389-4394 |
| DNASE1L3 | Deoxyribonuclease I-like 3 | SEQ ID NOS: 4395-4400 |
| DNASE2 | Deoxyribonuclease II, lysosomal | SEQ ID NOS: 4401-4402 |
| DNASE2B | Deoxyribonuclease II beta | SEQ ID NOS: 4403-4404 |
| DPEP1 | Dipeptidase 1 (renal) | SEQ ID NOS: 4405-4409 |
| DPEP2 | Dipeptidase 2 | SEQ ID NOS: 4410-4416 |
| DPEP3 | Dipeptidase 3 | SEQ ID NO: 4417 |
| DPF3 | D4, zinc and double PHD fingers, family 3 | SEQ ID NOS: 4418-4424 |
| DPP4 | Dipeptidyl-peptidase 4 | SEQ ID NOS: 4425-4429 |
| DPP7 | Dipeptidyl-peptidase 7 | SEQ ID NOS: 4430-4435 |
| DPT | Dermatopontin | SEQ ID NO: 4436 |
| DRAXIN | Dorsal inhibitory axon guidance protein | SEQ ID NO: 4437 |
| DSE | Dermatan sulfate epimerase | SEQ ID NOS: 4438-4446 |
| DSG2 | Desmoglein 2 | SEQ ID NOS: 4447-4448 |
| DSPP | Dentin sialophosphoprotein | SEQ ID NOS: 4449-4450 |
| DST | Dystonin | SEQ ID NOS: 4451-4469 |
| DUOX1 | Dual oxidase 1 | SEQ ID NOS: 4470-4474 |
| DYNLT3 | Dynein, light chain, Tctex-type 3 | SEQ ID NOS: 4475-4477 |
| E2F5 | E2F transcription factor 5, p130-binding | SEQ ID NOS: 4478-4484 |
| EBAG9 | Estrogen receptor binding site associated, antigen, 9 | SEQ ID NOS: 4485-4493 |
| EBI3 | Epstein-Barr virus induced 3 | SEQ ID NO: 4494 |
| ECHDC1 | Ethylmalonyl-CoA decarboxylase 1 | SEQ ID NOS: 4495-4513 |
| ECM1 | Extracellular matrix protein 1 | SEQ ID NOS: 4514-4516 |
| ECM2 | Extracellular matrix protein 2, female organ and | SEQ ID NOS: 4517-4520 |
| | adipocyte specific | |
| ECSIT | ECSIT signalling integrator | SEQ ID NOS: 4521-4532 |
| EDDM3A | Epididymal protein 3A | SEQ ID NO: 4533 |
| EDDM3B | Epididymal protein 3B | SEQ ID NO: 4534 |
| EDEM2 | ER degradation enhancer, mannosidase alpha-like 2 | SEQ ID NOS: 4535-4536 |
| EDEM3 | ER degradation enhancer, mannosidase alpha-like 3 | SEQ ID NOS: 4537-4539 |
| EDIL3 | EGF-like repeats and discoidin I-like domains 3 | SEQ ID NOS: 4540-4541 |
| EDN1 | Endothelin 1 | SEQ ID NO: 4542 |
| EDN2 | Endothelin 2 | SEQ ID NO: 4543 |
| EDN3 | Endothelin 3 | SEQ ID NOS: 4544-4549 |
| EDNRB | Endothelin receptor type B | SEQ ID NOS: 4550-4558 |
| EFEMP1 | EGF containing fibulin-like extracellular matrix | SEQ ID NOS: 4559-4569 |
| | protein 1 | |
| EFEMP2 | EGF containing fibulin-like extracellular matrix | SEQ ID NOS: 4570-4581 |
| | protein 2 | |
| EFNA1 | Ephrin-A1 | SEQ ID NOS: 4582-4583 |
| EFNA2 | Ephrin-A2 | SEQ ID NO: 4584 |
| EFNA4 | Ephrin-A4 | SEQ ID NOS: 4585-4587 |
| EGFL6 | EGF-like-domain, multiple 6 | SEQ ID NOS: 4588-4589 |
| EGFL7 | EGF-like-domain, multiple 7 | SEQ ID NOS: 4590-4594 |
| EGFL8 | EGF-like-domain, multiple 8 | SEQ ID NOS: 4595-4597 |
| EGFLAM | EGF-like, fibronectin type III and laminin G domains | SEQ ID NOS: 4598-4606 |
| EGFR | Epidermal growth factor receptor | SEQ ID NOS: 4607-4614 |
| EHBP1 | EH domain binding protein 1 | SEQ ID NOS: 4615-4626 |
| EHF | Ets homologous factor | SEQ ID NOS: 4627-4636 |
| EHMT1 | Euchromatic histone-lysine N-methyltransferase 1 | SEQ ID NOS: 4637-4662 |
| EHMT2 | Euchromatic histone-lysine N-methyltransferase 2 | SEQ ID NOS: 4663-4667 |
| EIF2AK1 | Eukaryotic translation initiation factor 2-alpha | SEQ ID NOS: 4668-4671 |
| | kinase 1 | |
| ELANE | Elastase, neutrophil expressed | SEQ ID NOS: 4672-4673 |
| ELN | Elastin | SEQ ID NOS: 4674-4696 |
| ELP2 | Elongator acetyltransferase complex subunit 2 | SEQ ID NOS: 4697-4709 |
| ELSPBP1 | Epididymal sperm binding protein 1 | SEQ ID NOS: 4710-4715 |
| EMC1 | ER membrane protein complex subunit 1 | SEQ ID NOS: 4716-4722 |
| EMC10 | ER membrane protein complex subunit 10 | SEQ ID NOS: 4723-4729 |
| EMC9 | ER membrane protein complex subunit 9 | SEQ ID NOS: 4730-4733 |
| EMCN | Endomucin | SEQ ID NOS: 4734-4738 |
| EMID1 | EMI domain containing 1 | SEQ ID NOS: 4739-4745 |
| EMILIN1 | Elastin microfibril interfacer 1 | SEQ ID NOS: 4746-4747 |
| EMILIN2 | Elastin microfibril interfacer 2 | SEQ ID NO: 4748 |
| EMILIN3 | Elastin microfibril interfacer 3 | SEQ ID NO: 4749 |
| ENAM | Enamelin | SEQ ID NO: 4750 |
| ENDOG | Endonuclease G | SEQ ID NO: 4751 |
| ENDOU | Endonuclease, polyU-specific | SEQ ID NOS: 4752-4754 |
| ENHO | Energy homeostasis associated | SEQ ID NO: 4755 |
| ENO4 | Enolase family member 4 | SEQ ID NOS: 4756-4760 |
| ENPP6 | Ectonucleotide pyrophosphatase/ | SEQ ID NOS: 4761-4762 |
| | phosphodiesterase 6 | |
| ENPP7 | Ectonucleotide pyrophosphatase/ | SEQ ID NOS: 4763-4764 |
| | phosphodiesterase 7 | |
| ENTPD5 | Ectonucleoside triphosphate diphosphohydrolase 5 | SEQ ID NOS: 4765-4769 |
| ENTPD8 | Ectonucleoside triphosphate diphosphohydrolase 8 | SEQ ID NOS: 4770-4773 |
| EOGT | EGF domain-specific O-linked N-acetylglucosamine | SEQ ID NOS: 4774-4781 |
| | (GlcNAc) transferase | |
| EPCAM | Epithelial cell adhesion molecule | SEQ ID NOS: 4782-4785 |
| EPDR1 | Ependymin related 1 | SEQ ID NOS: 4786-4789 |
| EPGN | Epithelial mitogen | SEQ ID NOS: 4790-4798 |
| EPHA10 | EPH receptor A10 | SEQ ID NOS: 4799-4806 |
| EPHA3 | EPH receptor A3 | SEQ ID NOS: 4807-4809 |
| EPHA4 | EPH receptor A4 | SEQ ID NOS: 4810-4819 |
| EPHA7 | EPH receptor A7 | SEQ ID NOS: 4820-4821 |
| EPHA8 | EPH receptor A8 | SEQ ID NOS: 4822-4823 |
| EPHB2 | EPH receptor B2 | SEQ ID NOS: 4824-4828 |
| EPHB4 | EPH receptor B4 | SEQ ID NOS: 4829-4831 |
| EPHX3 | Epoxide hydrolase 3 | SEQ ID NOS: 4832-4835 |
| EPO | Erythropoietin | SEQ ID NO: 4836 |
| EPPIN | Epididymal peptidase inhibitor | SEQ ID NOS: 4837-4839 |
| EPPIN- | EPPIN-WFDC6 readthrough | SEQ ID NO: 4840 |
| WFDC6 | | |
| EPS15 | Epidermal growth factor receptor pathway | SEQ ID NOS: 4841-4843 |
| | substrate 15 | |
| EPS8L1 | EPS8-like 1 | SEQ ID NOS: 4844-4849 |
| EPX | Eosinophil peroxidase | SEQ ID NO: 4850 |
| EPYC | Epiphycan | SEQ ID NOS: 4851-4852 |
| EQTN | Equatorin, sperm acrosome associated | SEQ ID NOS: 4853-4855 |
| ERAP1 | Endoplasmic reticulum aminopeptidase 1 | SEQ ID NOS: 4856-4861 |
| ERAP2 | Endoplasmic reticulum aminopeptidase 2 | SEQ ID NOS: 4862-4869 |
| ERBB3 | Erb-b2 receptor tyrosine kinase 3 | SEQ ID NOS: 4870-4883 |
| FAM132B | Family with sequence similarity 132, member B | SEQ ID NOS: 4884-4886 |
| ERLIN1 | ER lipid raft associated 1 | SEQ ID NOS: 4887-4889 |
| ERLIN2 | ER lipid raft associated 2 | SEQ ID NOS: 4890-4898 |
| ERN1 | Endoplasmic reticulum to nucleus signaling 1 | SEQ ID NOS: 4899-4900 |
| ERN2 | Endoplasmic reticulum to nucleus signaling 2 | SEQ ID NOS: 4901-4905 |
| ERO1A | Endoplasmic reticulum oxidoreductase alpha | SEQ ID NOS: 4906-4912 |
| ERO1B | Endoplasmic reticulum oxidoreductase beta | SEQ ID NOS: 4913-4915 |
| ERP27 | Endoplasmic reticulum protein 27 | SEQ ID NOS: 4916-4917 |
| ERP29 | Endoplasmic reticulum protein 29 | SEQ ID NOS: 4918-4921 |
| ERP44 | Endoplasmic reticulum protein 44 | SEQ ID NO: 4922 |
| ERV3-1 | Endogenous retrovirus group 3, member 1 | SEQ ID NO: 4923 |
| ESM1 | Endothelial cell-specific molecule 1 | SEQ ID NOS: 4924-4926 |
| ESRP1 | Epithelial splicing regulatory protein 1 | SEQ ID NOS: 4927-4935 |
| EXOG | Endo/exonuclease (5′-3′), endonuclease G-like | SEQ ID NOS: 4936-4949 |
| EXTL1 | Exostosin-like glycosyltransferase 1 | SEQ ID NO: 4950 |
| EXTL2 | Exostosin-like glycosyltransferase 2 | SEQ ID NOS: 4951-4955 |
| F10 | Coagulation factor X | SEQ ID NOS: 4956-4959 |
| F11 | Coagulation factor XI | SEQ ID NOS: 4960-4964 |
| F12 | Coagulation factor XII (Hageman factor) | SEQ ID NO: 4965 |
| F13B | Coagulation factor XIII, B polypeptide | SEQ ID NO: 4966 |
| F2 | Coagulation factor II (thrombin) | SEQ ID NOS: 4967-4969 |
| F2R | Coagulation factor II (thrombin) receptor | SEQ ID NOS: 4970-4971 |
| F2RL3 | Coagulation factor II (thrombin) receptor-like 3 | SEQ ID NOS: 4972-4973 |
| F5 | Coagulation factor V (proaccelerin, labile factor) | SEQ ID NOS: 4974-4975 |
| F7 | Coagulation factor VII (serum prothrombin | SEQ ID NOS: 4976-4979 |
| | conversion accelerator) | |
| F8 | Coagulation factor VIII, procoagulant component | SEQ ID NOS: 4980-4985 |
| F9 | Coagulation factor IX | SEQ ID NOS: 4986-4987 |
| FABP6 | Fatty acid binding protein 6, ileal | SEQ ID NOS: 4988-4990 |
| FAM107B | Family with sequence similarity 107, member B | SEQ ID NOS: 4991-5012 |
| FAM131A | Family with sequence similarity 131, member A | SEQ ID NOS: 5013-5021 |
| FAM171A1 | Family with sequence similarity 171, member A1 | SEQ ID NOS: 5022-5023 |
| FAM171B | Family with sequence similarity 171, member B | SEQ ID NOS: 5024-5025 |
| FAM172A | Family with sequence similarity 172, member A | SEQ ID NOS: 5026-5030 |
| FAM177A1 | Family with sequence similarity 177, member A1 | SEQ ID NOS: 5031-5040 |
| FAM180A | Family with sequence similarity 180, member A | SEQ ID NOS: 5041-5043 |
| FAM189A1 | Family with sequence similarity 189, member A1 | SEQ ID NOS: 5044-5045 |
| FAM198A | Family with sequence similarity 198, member A | SEQ ID NOS: 5046-5048 |
| FAM19A1 | Family with sequence similarity 19 (chemokine (C-C | SEQ ID NOS: 5049-5051 |
| | motif)-like), member A1 | |
| FAM19A2 | Family with sequence similarity 19 (chemokine (C-C | SEQ ID NOS: 5052-5059 |
| | motif)-like), member A2 | |
| FAM19A3 | Family with sequence similarity 19 (chemokine (C-C | SEQ ID NOS: 5060-5061 |
| | motif)-like), member A3 | |
| FAM19A4 | Family with sequence similarity 19 (chemokine (C-C | SEQ ID NOS: 5062-5064 |
| | motif)-like), member A4 | |
| FAM19A5 | Family with sequence similarity 19 (chemokine (C-C | SEQ ID NOS: 5065-5068 |
| | motif)-like), member A5 | |
| FAM20A | Family with sequence similarity 20, member A | SEQ ID NOS: 5069-5072 |
| FAM20C | Family with sequence similarity 20, member C | SEQ ID NO: 5073 |
| FAM213A | Family with sequence similarity 213, member A | SEQ ID NOS: 5074-5079 |
| FAM46B | Family with sequence similarity 46, member B | SEQ ID NO: 5080 |
| FAM57A | Family with sequence similarity 57, member A | SEQ ID NOS: 5081-5086 |
| FAM78A | Family with sequence similarity 78, member A | SEQ ID NOS: 5087-5089 |
| FAM96A | Family with sequence similarity 96, member A | SEQ ID NOS: 5090-5094 |
| FAM9B | Family with sequence similarity 9, member B | SEQ ID NOS: 5095-5098 |
| FAP | Fibroblast activation protein, alpha | SEQ ID NOS: 5099-5105 |
| FAS | Fas cell surface death receptor | SEQ ID NOS: 5106-5115 |
| FAT1 | FAT atypical cadherin 1 | SEQ ID NOS: 5116-5122 |
| FBLN1 | Fibulin 1 | SEQ ID NOS: 5123-5135 |
| FBLN2 | Fibulin 2 | SEQ ID NOS: 5136-5141 |
| FBLN5 | Fibulin 5 | SEQ ID NOS: 5142-5147 |
| FBLN7 | Fibulin 7 | SEQ ID NOS: 5148-5153 |
| FBN1 | Fibrillin 1 | SEQ ID NOS: 5154-5157 |
| FBN2 | Fibrillin 2 | SEQ ID NOS: 5158-5163 |
| FBN3 | Fibrillin 3 | SEQ ID NOS: 5164-5168 |
| FBXW7 | F-box and WD repeat domain containing 7, E3 | SEQ ID NOS: 5169-5179 |
| | ubiquitin protein ligase | |
| FCAR | Fc fragment of IgA receptor | SEQ ID NOS: 5180-5189 |
| FCGBP | Fc fragment of IgG binding protein | SEQ ID NOS: 5190-5192 |
| FCGR1B | Fc fragment of IgG, high affinity Ib, receptor (CD64) | SEQ ID NOS: 5193-5198 |
| FCGR3A | Fc fragment of IgG, low affinity IIIa, receptor (CD16a) | SEQ ID NOS: 5199-5205 |
| FCGRT | Fc fragment of IgG, receptor, transporter, alpha | SEQ ID NOS: 5206-5216 |
| FCMR | Fc fragment of IgM receptor | SEQ ID NOS: 5217-5223 |
| FCN1 | Ficolin (collagen/fibrinogen domain containing) 1 | SEQ ID NOS: 5224-5225 |
| FCN2 | Ficolin (collagen/fibrinogen domain containing | SEQ ID NOS: 5226-5227 |
| | lectin) 2 | |
| FCN3 | Ficolin (collagen/fibrinogen domain containing) 3 | SEQ ID NOS: 5228-5229 |
| FCRL1 | Fc receptor-like 1 | SEQ ID NOS: 5230-5232 |
| FCRL3 | Fc receptor-like 3 | SEQ ID NOS: 5233-5238 |
| FCRL5 | Fc receptor-like 5 | SEQ ID NOS: 5239-5241 |
| FCRLA | Fc receptor-like A | SEQ ID NOS: 5242-5253 |
| FCRLB | Fc receptor-like B | SEQ ID NOS: 5254-5258 |
| FDCSP | Follicular dendritic cell secreted protein | SEQ ID NO: 5259 |
| FETUB | Fetuin B | SEQ ID NOS: 5260-5266 |
| FGA | Fibrinogen alpha chain | SEQ ID NOS: 5267-5269 |
| FGB | Fibrinogen beta chain | SEQ ID NOS: 5270-5272 |
| FGF10 | Fibroblast growth factor 10 | SEQ ID NOS: 5273-5274 |
| FGF17 | Fibroblast growth factor 17 | SEQ ID NOS: 5275-5276 |
| FGF18 | Fibroblast growth factor 18 | SEQ ID NO: 5277 |
| FGF19 | Fibroblast growth factor 19 | SEQ ID NO: 5278 |
| FGF21 | Fibroblast growth factor 21 | SEQ ID NOS: 5279-5280 |
| FGF22 | Fibroblast growth factor 22 | SEQ ID NOS: 5281-5282 |
| FGF23 | Fibroblast growth factor 23 | SEQ ID NO: 5283 |
| FGF3 | Fibroblast growth factor 3 | SEQ ID NO: 5284 |
| FGF4 | Fibroblast growth factor 4 | SEQ ID NO: 5285 |
| FGF5 | Fibroblast growth factor 5 | SEQ ID NOS: 5286-5288 |
| FGF7 | Fibroblast growth factor 7 | SEQ ID NOS: 5289-5293 |
| FGF8 | Fibroblast growth factor 8 (androgen-induced) | SEQ ID NOS: 5294-5299 |
| FGFBP1 | Fibroblast growth factor binding protein 1 | SEQ ID NO: 5300 |
| FGFBP2 | Fibroblast growth factor binding protein 2 | SEQ ID NO: 5301 |
| FGFBP3 | Fibroblast growth factor binding protein 3 | SEQ ID NO: 5302 |
| FGFR1 | Fibroblast growth factor receptor 1 | SEQ ID NOS: 5303-5325 |
| FGFR2 | Fibroblast growth factor receptor 2 | SEQ ID NOS: 5326-5347 |
| FGFR3 | Fibroblast growth factor receptor 3 | SEQ ID NOS: 5348-5355 |
| FGFR4 | Fibroblast growth factor receptor 4 | SEQ ID NOS: 5356-5365 |
| FGFRL1 | Fibroblast growth factor receptor-like 1 | SEQ ID NOS: 5366-5371 |
| FGG | Fibrinogen gamma chain | SEQ ID NOS: 5372-5377 |
| FGL1 | Fibrinogen-like 1 | SEQ ID NOS: 5378-5384 |
| FGL2 | Fibrinogen-like 2 | SEQ ID NOS: 5385-5386 |
| FHL1 | Four and a half LIM domains 1 | SEQ ID NOS: 5387-5414 |
| FHOD3 | Formin homology 2 domain containing 3 | SEQ ID NOS: 5415-5421 |
| FIBIN | Fin bud initiation factor homolog (zebrafish) | SEQ ID NO: 5422 |
| FICD | FIC domain containing | SEQ ID NOS: 5423-5426 |
| FJX1 | Four jointed box 1 | SEQ ID NO: 5427 |
| FKBP10 | FK506 binding protein 10, 65 kDa | SEQ ID NOS: 5428-5433 |
| FKBP11 | FK506 binding protein 11, 19 kDa | SEQ ID NOS: 5434-5440 |
| FKBP14 | FK506 binding protein 14, 22 kDa | SEQ ID NOS: 5441-5443 |
| FKBP2 | FK506 binding protein 2, 13 kDa | SEQ ID NOS: 5444-5447 |
| FKBP7 | FK506 binding protein 7 | SEQ ID NOS: 5448-5453 |
| FKBP9 | FK506 binding protein 9, 63 kDa | SEQ ID NOS: 5454-5457 |
| FLT1 | Fms-related tyrosine kinase 1 | SEQ ID NOS: 5458-5466 |
| FLT4 | Fms-related tyrosine kinase 4 | SEQ ID NOS: 5467-5471 |
| FMO1 | Flavin containing monooxygenase 1 | SEQ ID NOS: 5472-5476 |
| FMO2 | Flavin containing monooxygenase 2 (non-functional) | SEQ ID NOS: 5477-5479 |
| FMO3 | Flavin containing monooxygenase 3 | SEQ ID NOS: 5480-5482 |
| FMO5 | Flavin containing monooxygenase 5 | SEQ ID NOS: 5483-5489 |
| FMOD | Fibromodulin | SEQ ID NO: 5490 |
| FN1 | Fibronectin 1 | SEQ ID NOS: 5491-5503 |
| FNDC1 | Fibronectin type III domain containing 1 | SEQ ID NOS: 5504-5505 |
| FNDC7 | Fibronectin type III domain containing 7 | SEQ ID NOS: 5506-5507 |
| FOCAD | Focadhesin | SEQ ID NOS: 5508-5514 |
| FOLR2 | Folate receptor 2 (fetal) | SEQ ID NOS: 5515-5524 |
| FOLR3 | Folate receptor 3 (gamma) | SEQ ID NOS: 5525-5529 |
| FOXRED2 | FAD-dependent oxidoreductase domain containing 2 | SEQ ID NOS: 5530-5533 |
| FP325331.1 | Uncharacterized protein UNQ6126/PRO20091 | SEQ ID NO: 5534 |
| CH507- | | SEQ ID NOS: 5535-5541 |
| 9B2.3 | | |
| FPGS | Folylpolyglutamate synthase | SEQ ID NOS: 5542-5548 |
| FRAS1 | Fraser extracellular matrix complex subunit 1 | SEQ ID NOS: 5549-5554 |
| FREM1 | FRAS1 related extracellular matrix 1 | SEQ ID NOS: 5555-5559 |
| FREM3 | FRAS1 related extracellular matrix 3 | SEQ ID NO: 5560 |
| FRMPD2 | FERM and PDZ domain containing 2 | SEQ ID NOS: 5561-5564 |
| FRZB | Frizzled-related protein | SEQ ID NO: 5565 |
| FSHB | Follicle stimulating hormone, beta polypeptide | SEQ ID NOS: 5566-5568 |
| FSHR | Follicle stimulating hormone receptor | SEQ ID NOS: 5569-5572 |
| FST | Follistatin | SEQ ID NOS: 5573-5576 |
| FSTL1 | Follistatin-like 1 | SEQ ID NOS: 5577-5580 |
| FSTL3 | Follistatin-like 3 (secreted glycoprotein) | SEQ ID NOS: 5581-5586 |
| FSTL4 | Follistatin-like 4 | SEQ ID NOS: 5587-5589 |
| FSTL5 | Follistatin-like 5 | SEQ ID NOS: 5590-5592 |
| FTCDNL1 | Formiminotransferase cyclodeaminase N-terminal | SEQ ID NOS: 5593-5596 |
| | like | |
| FUCA1 | Fucosidase, alpha-L-1, tissue | SEQ ID NO: 5597 |
| FUCA2 | Fucosidase, alpha-L-2, plasma | SEQ ID NOS: 5598-5599 |
| FURIN | Furin (paired basic amino acid cleaving enzyme) | SEQ ID NOS: 5600-5606 |
| FUT10 | Fucosyltransferase 10 (alpha (1,3) | SEQ ID NOS: 5607-5609 |
| | fucosyltransferase) | |
| FUT11 | Fucosyltransferase 11 (alpha (1,3) | SEQ ID NOS: 5610-5611 |
| | fucosyltransferase) | |
| FXN | Frataxin | SEQ ID NOS: 5612-5619 |
| FXR1 | Fragile X mental retardation, autosomal homolog 1 | SEQ ID NOS: 5620-5632 |
| FXYD3 | FXYD domain containing ion transport regulator 3 | SEQ ID NOS: 5633-5645 |
| GABBR1 | Gamma-aminobutyric acid (GABA) B receptor, 1 | SEQ ID NOS: 5646-5657 |
| GABRA1 | Gamma-aminobutyric acid (GABA) A receptor, | SEQ ID NOS: 5658-5673 |
| | alpha 1 | |
| GABRA2 | Gamma-aminobutyric acid (GABA) A receptor, | SEQ ID NOS: 5674-5688 |
| | alpha 2 | |
| GABRA5 | Gamma-aminobutyric acid (GABA) A receptor, | SEQ ID NOS: 5689-5697 |
| | alpha 5 | |
| GABRG3 | Gamma-aminobutyric acid (GABA) A receptor, | SEQ ID NOS: 5698-5703 |
| | gamma 3 | |
| GABRP | Gamma-aminobutyric acid (GABA) A receptor, pi | SEQ ID NOS: 5704-5712 |
| GAL | Galanin/GMAP prepropeptide | SEQ ID NO: 5713 |
| GAL3ST1 | Galactose-3-O-sulfotransferase 1 | SEQ ID NOS: 5714-5735 |
| GAL3ST2 | Galactose-3-O-sulfotransferase 2 | SEQ ID NO: 5736 |
| GAL3ST3 | Galactose-3-O-sulfotransferase 3 | SEQ ID NOS: 5737-5738 |
| GALC | Galactosylceramidase | SEQ ID NOS: 5739-5748 |
| GALNS | Galactosamine (N-acetyl)-6-sulfatase | SEQ ID NOS: 5749-5754 |
| GALNT10 | Polypeptide N-acetylgalactosaminyltransferase 10 | SEQ ID NOS: 5755-5758 |
| GALNT12 | Polypeptide N-acetylgalactosaminyltransferase 12 | SEQ ID NOS: 5759-5760 |
| GALNT15 | Polypeptide N-acetylgalactosaminyltransferase 15 | SEQ ID NOS: 5761-5764 |
| GALNT2 | Polypeptide N-acetylgalactosaminyltransferase 2 | SEQ ID NO: 5765 |
| GALNT6 | Polypeptide N-acetylgalactosaminyltransferase 6 | SEQ ID NOS: 5766-5777 |
| GALNT8 | Polypeptide N-acetylgalactosaminyltransferase 8 | SEQ ID NOS: 5778-5781 |
| GALNTL6 | Polypeptide N-acetylgalactosaminyltransferase- | SEQ ID NOS: 5782-5785 |
| | like 6 | |
| GALP | Galanin-like peptide | SEQ ID NOS: 5786-5788 |
| GANAB | Glucosidase, alpha; neutral AB | SEQ ID NOS: 5789-5797 |
| GARS | Glycyl-tRNA synthetase | SEQ ID NOS: 5798-5801 |
| GAS1 | Growth arrest-specific 1 | SEQ ID NO: 5802 |
| GAS6 | Growth arrest-specific 6 | SEQ ID NO: 5803 |
| GAST | Gastrin | SEQ ID NO: 5804 |
| PDDC1 | Parkinson disease 7 domain containing 1 | SEQ ID NOS: 5805-5813 |
| GBA | Glucosidase, beta, acid | SEQ ID NOS: 5814-5817 |
| GBGT1 | Globoside alpha-1,3-N- | SEQ ID NOS: 5818-5826 |
| | acetylgalactosaminyltransferase 1 | |
| GC | Group-specific component (vitamin D binding | SEQ ID NOS: 5827-5831 |
| | protein) | |
| GCG | Glucagon | SEQ ID NOS: 5832-5833 |
| GCGR | Glucagon receptor | SEQ ID NOS: 5834-5836 |
| GCNT7 | Glucosaminyl (N-acetyl) transferase family | SEQ ID NOS: 5837-5838 |
| | member 7 | |
| GCSH | Glycine cleavage system protein H (aminomethyl | SEQ ID NOS: 5839-5847 |
| | carrier) | |
| GDF1 | Growth differentiation factor 1 | SEQ ID NO: 5848 |
| GDF10 | Growth differentiation factor 10 | SEQ ID NO: 5849 |
| GDF11 | Growth differentiation factor 11 | SEQ ID NOS: 5850-5851 |
| GDF15 | Growth differentiation factor 15 | SEQ ID NOS: 5852-5854 |
| GDF2 | Growth differentiation factor 2 | SEQ ID NO: 5855 |
| GDF3 | Growth differentiation factor 3 | SEQ ID NO: 5856 |
| GDF5 | Growth differentiation factor 5 | SEQ ID NOS: 5857-5858 |
| GDF6 | Growth differentiation factor 6 | SEQ ID NOS: 5859-5861 |
| GDF7 | Growth differentiation factor 7 | SEQ ID NO: 5862 |
| GDF9 | Growth differentiation factor 9 | SEQ ID NOS: 5863-5867 |
| GDNF | Glial cell derived neurotrophic factor | SEQ ID NOS: 5868-5875 |
| GFOD2 | Glucose-fructose oxidoreductase domain | SEQ ID NOS: 5876-5881 |
| | containing 2 | |
| GFPT2 | Glutamine-fructose-6-phosphate transaminase 2 | SEQ ID NOS: 5882-5884 |
| GFRA2 | GDNF family receptor alpha 2 | SEQ ID NOS: 5885-5891 |
| GFRA4 | GDNF family receptor alpha 4 | SEQ ID NOS: 5892-5894 |
| GGA2 | Golgi-associated, gamma adaptin ear containing, | SEQ ID NOS: 5895-5903 |
| | ARF binding protein 2 | |
| GGH | Gamma-glutamyl hydrolase (conjugase, | SEQ ID NO: 5904 |
| | folylpolygammaglutamyl hydrolase) | |
| GGT1 | Gamma-glutamyltransferase 1 | SEQ ID NOS: 5905-5927 |
| GGT5 | Gamma-glutamyltransferase 5 | SEQ ID NOS: 5928-5932 |
| GH1 | Growth hormone 1 | SEQ ID NOS: 5933-5937 |
| GH2 | Growth hormone 2 | SEQ ID NOS: 5938-5942 |
| GHDC | GH3 domain containing | SEQ ID NOS: 5943-5950 |
| GHRH | Growth hormone releasing hormone | SEQ ID NOS: 5951-5953 |
| GHRHR | Growth hormone releasing hormone receptor | SEQ ID NOS: 5954-5959 |
| GHRL | Ghrelin/obestatin prepropeptide | SEQ ID NOS: 5960-5970 |
| GIF | Gastric intrinsic factor (vitamin B synthesis) | SEQ ID NOS: 5971-5972 |
| GIP | Gastric inhibitory polypeptide | SEQ ID NO: 5973 |
| GKN1 | Gastrokine 1 | SEQ ID NO: 5974 |
| GKN2 | Gastrokine 2 | SEQ ID NOS: 5975-5976 |
| GLA | Galactosidase, alpha | SEQ ID NOS: 5977-5978 |
| GLB1 | Galactosidase, beta 1 | SEQ ID NOS: 5979-5987 |
| GLB1L | Galactosidase, beta 1-like | SEQ ID NOS: 5988-5995 |
| GLB1L2 | Galactosidase, beta 1-like 2 | SEQ ID NOS: 5996-5997 |
| GLCE | Glucuronic acid epimerase | SEQ ID NOS: 5998-5999 |
| GLG1 | Golgi glycoprotein 1 | SEQ ID NOS: 6000-6007 |
| GLIPR1 | GLI pathogenesis-related 1 | SEQ ID NOS: 6008-6011 |
| GLIPR1L1 | GLI pathogenesis-related 1 like 1 | SEQ ID NOS: 6012-6015 |
| GLIS3 | GLIS family zinc finger 3 | SEQ ID NOS: 6016-6024 |
| GLMP | Glycosylated lysosomal membrane protein | SEQ ID NOS: 6025-6033 |
| GLRB | Glycine receptor, beta | SEQ ID NOS: 6034-6039 |
| GLS | Glutaminase | SEQ ID NOS: 6040-6047 |
| GLT6D1 | Glycosyltransferase 6 domain containing 1 | SEQ ID NOS: 6048-6049 |
| GLTPD2 | Glycolipid transfer protein domain containing 2 | SEQ ID NO: 6050 |
| GLUD1 | Glutamate dehydrogenase 1 | SEQ ID NO: 6051 |
| GM2A | GM2 ganglioside activator | SEQ ID NOS: 6052-6054 |
| GML | Glycosylphosphatidylinositol anchored molecule like | SEQ ID NOS: 6055-6056 |
| GNAS | GNAS complex locus | SEQ ID NOS: 6057-6078 |
| GNLY | Granulysin | SEQ ID NOS: 6079-6082 |
| GNPTG | N-acetylglucosamine-1-phosphate transferase, | SEQ ID NOS: 6083-6087 |
| | gamma subunit | |
| GNRH1 | Gonadotropin-releasing hormone 1 (luteinizing- | SEQ ID NOS: 6088-6089 |
| | releasing hormone) | |
| GNRH2 | Gonadotropin-releasing hormone 2 | SEQ ID NOS: 6090-6093 |
| GNS | Glucosamine (N-acetyl)-6-sulfatase | SEQ ID NOS: 6094-6099 |
| GOLM1 | Golgi membrane protein 1 | SEQ ID NOS: 6100-6104 |
| GORAB | Golgin, RAB6-interacting | SEQ ID NOS: 6105-6107 |
| GOT2 | Glutamic-oxaloacetic transaminase 2, mitochondrial | SEQ ID NOS: 6108-6110 |
| GP2 | Glycoprotein 2 (zymogen granule membrane) | SEQ ID NOS: 6111-6119 |
| GP6 | Glycoprotein VI (platelet) | SEQ ID NOS: 6120-6123 |
| GPC2 | Glypican 2 | SEQ ID NOS: 6124-6125 |
| GPC5 | Glypican 5 | SEQ ID NOS: 6126-6128 |
| GPC6 | Glypican 6 | SEQ ID NOS: 6129-6130 |
| GPD2 | Glycerol-3-phosphate dehydrogenase 2 | SEQ ID NOS: 6131-6139 |
| | (mitochondrial) | |
| GPER1 | G protein-coupled estrogen receptor 1 | SEQ ID NOS: 6140-6146 |
| GPHA2 | Glycoprotein hormone alpha 2 | SEQ ID NOS: 6147-6149 |
| GPHB5 | Glycoprotein hormone beta 5 | SEQ ID NOS: 6150-6151 |
| GPIHBP1 | Glycosylphosphatidylinositol anchored high density | SEQ ID NO: 6152 |
| | lipoprotein binding protein 1 | |
| GPLD1 | Glycosylphosphatidylinositol specific phospholipase | SEQ ID NO: 6153 |
| | D1 | |
| GPNMB | Glycoprotein (transmembrane) nmb | SEQ ID NOS: 6154-6156 |
| GPR162 | G protein-coupled receptor 162 | SEQ ID NOS: 6157-6160 |
| GPX3 | Glutathione peroxidase 3 | SEQ ID NOS: 6161-6168 |
| GPX4 | Glutathione peroxidase 4 | SEQ ID NOS: 6169-6179 |
| GPX5 | Glutathione peroxidase 5 | SEQ ID NOS: 6180-6181 |
| GPX6 | Glutathione peroxidase 6 | SEQ ID NOS: 6182-6184 |
| GPX7 | Glutathione peroxidase 7 | SEQ ID NO: 6185 |
| GREM1 | Gremlin 1, DAN family BMP antagonist | SEQ ID NOS: 6186-6188 |
| GREM2 | Gremlin 2, DAN family BMP antagonist | SEQ ID NO: 6189 |
| GRHL3 | Grainyhead-like transcription factor 3 | SEQ ID NOS: 6190-6195 |
| GRIA2 | Glutamate receptor, ionotropic, AMPA 2 | SEQ ID NOS: 6196-6207 |
| GRIA3 | Glutamate receptor, ionotropic, AMPA 3 | SEQ ID NOS: 6208-6213 |
| GRIA4 | Glutamate receptor, ionotropic, AMPA 4 | SEQ ID NOS: 6214-6225 |
| GRIK2 | Glutamate receptor, ionotropic, kainate 2 | SEQ ID NOS: 6226-6234 |
| GRIN2B | Glutamate receptor, ionotropic, N-methyl D- | SEQ ID NOS: 6235-6238 |
| | aspartate 2B | |
| GRM2 | Glutamate receptor, metabotropic 2 | SEQ ID NOS: 6239-6242 |
| GRM3 | Glutamate receptor, metabotropic 3 | SEQ ID NOS: 6243-6247 |
| GRM5 | Glutamate receptor, metabotropic 5 | SEQ ID NOS: 6248-6252 |
| GRN | Granulin | SEQ ID NOS: 6253-6268 |
| GRP | Gastrin-releasing peptide | SEQ ID NOS: 6269-6273 |
| DFNA5 | Deafness, autosomal dominant 5 | SEQ ID NOS: 6274-6282 |
| GSG1 | Germ cell associated 1 | SEQ ID NOS: 6283-6291 |
| GSN | Gelsolin | SEQ ID NOS: 6292-6300 |
| GTDC1 | Glycosyltransferase-like domain containing 1 | SEQ ID NOS: 6301-6314 |
| GTPBP10 | GTP-binding protein 10 (putative) | SEQ ID NOS: 6315-6323 |
| GUCA2A | Guanylate cyclase activator 2A (guanylin) | SEQ ID NO: 6324 |
| GUCA2B | Guanylate cyclase activator 2B (uroguanylin) | SEQ ID NO: 6325 |
| GUSB | Glucuronidase, beta | SEQ ID NOS: 6326-6330 |
| GVQW1 | GVQW motif containing 1 | SEQ ID NO: 6331 |
| GXYLT1 | Glucoside xylosyltransferase 1 | SEQ ID NOS: 6332-6333 |
| GXYLT2 | Glucoside xylosyltransferase 2 | SEQ ID NOS: 6334-6336 |
| GYPB | Glycophorin B (MNS blood group) | SEQ ID NOS: 6337-6345 |
| GZMA | Granzyme A (granzyme 1, cytotoxic T-lymphocyte- | SEQ ID NO: 6346 |
| | associated serine esterase 3) | |
| GZMB | Granzyme B (granzyme 2, cytotoxic T-lymphocyte- | SEQ ID NOS: 6347-6355 |
| | associated serine esterase 1) | |
| GZMH | Granzyme H (cathepsin G-like 2, protein h-CCPX) | SEQ ID NOS: 6356-6358 |
| GZMK | Granzyme K (granzyme 3; tryptase II) | SEQ ID NO: 6359 |
| GZMM | Granzyme M (lymphocyte met-ase 1) | SEQ ID NOS: 6360-6361 |
| H6PD | Hexose-6-phosphate dehydrogenase (glucose 1- | SEQ ID NOS: 6362-6363 |
| | dehydrogenase) | |
| HABP2 | Hyaluronan binding protein 2 | SEQ ID NOS: 6364-6365 |
| HADHB | Hydroxyacyl-CoA dehydrogenase/3-ketoacyl-CoA | SEQ ID NOS: 6366-6372 |
| | thiolase/enoyl-CoA hydratase (trifunctional protein), | |
| | beta subunit | |
| HAMP | Hepcidin antimicrobial peptide | SEQ ID NOS: 6373-6374 |
| HAPLN1 | Hyaluronan and proteoglycan link protein 1 | SEQ ID NOS: 6375-6381 |
| HAPLN2 | Hyaluronan and proteoglycan link protein 2 | SEQ ID NOS: 6382-6383 |
| HAPLN3 | Hyaluronan and proteoglycan link protein 3 | SEQ ID NOS: 6384-6387 |
| HAPLN4 | Hyaluronan and proteoglycan link protein 4 | SEQ ID NO: 6388 |
| HARS2 | Histidyl-tRNA synthetase 2, mitochondrial | SEQ ID NOS: 6389-6404 |
| HAVCR1 | Hepatitis A virus cellular receptor 1 | SEQ ID NOS: 6405-6409 |
| HCCS | Holocytochrome c synthase | SEQ ID NOS: 6410-6412 |
| HCRT | Hypocretin (orexin) neuropeptide precursor | SEQ ID NO: 6413 |
| CECR5 | Cat eye syndrome chromosome region, candidate 5 | SEQ ID NOS: 6414-6416 |
| HEATR5A | HEAT repeat containing 5A | SEQ ID NOS: 6417-6423 |
| HEPH | Hephaestin | SEQ ID NOS: 6424-6431 |
| HEXA | Hexosaminidase A (alpha polypeptide) | SEQ ID NOS: 6432-6441 |
| HEXB | Hexosaminidase B (beta polypeptide) | SEQ ID NOS: 6442-6447 |
| HFE2 | Hemochromatosis type 2 (juvenile) | SEQ ID NOS: 6448-6454 |
| HGF | Hepatocyte growth factor (hepapoietin A; scatter | SEQ ID NOS: 6455-6465 |
| | factor) | |
| HGFAC | HGF activator | SEQ ID NOS: 6466-6467 |
| HHIP | Hedgehog interacting protein | SEQ ID NOS: 6468-6469 |
| HHIPL1 | HHIP-like 1 | SEQ ID NOS: 6470-6471 |
| HHIPL2 | HHIP-like 2 | SEQ ID NO: 6472 |
| HHLA1 | HERV-H LTR-associating 1 | SEQ ID NOS: 6473-6474 |
| HHLA2 | HERV-H LTR-associating 2 | SEQ ID NOS: 6475-6485 |
| HIBADH | 3-hydroxyisobutyrate dehydrogenase | SEQ ID NOS: 6486-6488 |
| HINT2 | Histidine triad nucleotide binding protein 2 | SEQ ID NO: 6489 |
| HLA-A | Major histocompatibility complex, class I, A | SEQ ID NOS: 6490-6494 |
| HLA-C | Major histocompatibility complex, class I, C | SEQ ID NOS: 6495-6499 |
| HLA-DOA | Major histocompatibility complex, class II, DO alpha | SEQ ID NOS: 6500-6501 |
| HLA-DPA1 | Major histocompatibility complex, class II, DP | SEQ ID NOS: 6502-6505 |
| | alpha 1 | |
| HLA-DQA1 | Major histocompatibility complex, class II, DQ | SEQ ID NOS: 6506-6511 |
| | alpha 1 | |
| HLA-DQB1 | Major histocompatibility complex, class II, DQ beta 1 | SEQ ID NOS: 6512-6517 |
| HLA-DQB2 | Major histocompatibility complex, class II, DQ beta 2 | SEQ ID NOS: 6518-6521 |
| HMCN1 | Hemicentin 1 | SEQ ID NOS: 6522-6523 |
| HMCN2 | Hemicentin 2 | SEQ ID NOS: 6524-6527 |
| HMGCL | 3-hydroxymethyl-3-methylglutaryl-CoA lyase | SEQ ID NOS: 6528-6531 |
| HMSD | Histocompatibility (minor) serpin domain containing | SEQ ID NOS: 6532-6533 |
| HP | Haptoglobin | SEQ ID NOS: 6534-6547 |
| HPR | Haptoglobin-related protein | SEQ ID NOS: 6548-6550 |
| HPSE | Heparanase | SEQ ID NOS: 6551-6557 |
| HPSE2 | Heparanase 2 (inactive) | SEQ ID NOS: 6558-6563 |
| HPX | Hemopexin | SEQ ID NOS: 6564-6565 |
| HRC | Histidine rich calcium binding protein | SEQ ID NOS: 6566-6568 |
| HRG | Histidine-rich glycoprotein | SEQ ID NO: 6569 |
| HS2ST1 | Heparan sulfate 2-O-sulfotransferase 1 | SEQ ID NOS: 6570-6572 |
| HS3ST1 | Heparan sulfate (glucosamine) 3-O- | SEQ ID NOS: 6573-6575 |
| | sulfotransferase 1 | |
| HS6ST1 | Heparan sulfate 6-O-sulfotransferase 1 | SEQ ID NO: 6576 |
| HS6ST3 | Heparan sulfate 6-O-sulfotransferase 3 | SEQ ID NOS: 6577-6578 |
| HSD11B1L | Hydroxysteroid (11-beta) dehydrogenase 1-like | SEQ ID NOS: 6579-6597 |
| HSD17811 | Hydroxysteroid (17-beta) dehydrogenase 11 | SEQ ID NOS: 6598-6599 |
| HSD17B7 | Hydroxysteroid (17-beta) dehydrogenase 7 | SEQ ID NOS: 6600-6604 |
| HSP90B1 | Heat shock protein 90 kDa beta (Grp94), member 1 | SEQ ID NOS: 6605-6610 |
| HSPA13 | Heat shock protein 70 kDa family, member 13 | SEQ ID NO: 6611 |
| HSPA5 | Heat shock 70 kDa protein 5 (glucose-regulated | SEQ ID NO: 6612 |
| | protein, 78 kDa) | |
| HSPG2 | Heparan sulfate proteoglycan 2 | SEQ ID NOS: 6613-6617 |
| HTATIP2 | HIV-1 Tat interactive protein 2, 30 kDa | SEQ ID NOS: 6618-6625 |
| HTN1 | Histatin 1 | SEQ ID NOS: 6626-6628 |
| HTN3 | Histatin 3 | SEQ ID NOS: 6629-6631 |
| HTRA1 | HtrA serine peptidase 1 | SEQ ID NOS: 6632-6633 |
| HTRA3 | HtrA serine peptidase 3 | SEQ ID NOS: 6634-6635 |
| HTRA4 | HtrA serine peptidase 4 | SEQ ID NO: 6636 |
| HYAL1 | Hyaluronoglucosaminidase 1 | SEQ ID NOS: 6637-6645 |
| HYAL2 | Hyaluronoglucosaminidase 2 | SEQ ID NOS: 6646-6654 |
| HYAL3 | Hyaluronoglucosaminidase 3 | SEQ ID NOS: 6655-6661 |
| HYOU1 | Hypoxia up-regulated 1 | SEQ ID NOS: 6662-6676 |
| IAPP | Islet amyloid polypeptide | SEQ ID NOS: 6677-6681 |
| IBSP | Integrin-binding sialoprotein | SEQ ID NO: 6682 |
| ICAM1 | Intercellular adhesion molecule 1 | SEQ ID NOS: 6683-6685 |
| ICAM2 | Intercellular adhesion molecule 2 | SEQ ID NOS: 6686-6696 |
| ICAM4 | Intercellular adhesion molecule 4 (Landsteiner- | SEQ ID NOS: 6697-6699 |
| | Wiener blood group) | |
| ID1 | Inhibitor of DNA binding 1, dominant negative helix- | SEQ ID NOS: 6700-6701 |
| | loop-helix protein | |
| IDE | Insulin-degrading enzyme | SEQ ID NOS: 6702-6705 |
| IDNK | IdnK, gluconokinase homolog (E. coli) | SEQ ID NOS: 6706-6711 |
| IDS | Iduronate 2-sulfatase | SEQ ID NOS: 6712-6717 |
| IDUA | Iduronidase, alpha-L- | SEQ ID NOS: 6718-6723 |
| IFI27L2 | Interferon, alpha-inducible protein 27-like 2 | SEQ ID NOS: 6724-6725 |
| IFI30 | Interferon, gamma-inducible protein 30 | SEQ ID NOS: 6726-6727 |
| IFNA1 | Interferon, alpha 1 | SEQ ID NO: 6728 |
| IFNA10 | Interferon, alpha 10 | SEQ ID NO: 6729 |
| IFNA13 | Interferon, alpha 13 | SEQ ID NOS: 6730-6731 |
| IFNA14 | Interferon, alpha 14 | SEQ ID NO: 6732 |
| IFNA16 | Interferon, alpha 16 | SEQ ID NO: 6733 |
| IFNA17 | Interferon, alpha 17 | SEQ ID NO: 6734 |
| IFNA2 | Interferon, alpha 2 | SEQ ID NO: 6735 |
| IFNA21 | Interferon, alpha 21 | SEQ ID NO: 6736 |
| IFNA4 | Interferon, alpha 4 | SEQ ID NO: 6737 |
| IFNA5 | Interferon, alpha 5 | SEQ ID NO: 6738 |
| IFNA6 | Interferon, alpha 6 | SEQ ID NOS: 6739-6740 |
| IFNA7 | Interferon, alpha 7 | SEQ ID NO: 6741 |
| IFNA8 | Interferon, alpha 8 | SEQ ID NO: 6742 |
| IFNAR1 | Interferon (alpha, beta and omega) receptor 1 | SEQ ID NOS: 6743-6744 |
| IFNB1 | Interferon, beta 1, fibroblast | SEQ ID NO: 6745 |
| IFNE | Interferon, epsilon | SEQ ID NO: 6746 |
| IFNG | Interferon, gamma | SEQ ID NO: 6747 |
| IFNGR1 | Interferon gamma receptor 1 | SEQ ID NOS: 6748-6758 |
| IFNL1 | Interferon, lambda 1 | SEQ ID NO: 6759 |
| IFNL2 | Interferon, lambda 2 | SEQ ID NO: 6760 |
| IFNL3 | Interferon, lambda 3 | SEQ ID NOS: 6761-6762 |
| IFNLR1 | Interferon, lambda receptor 1 | SEQ ID NOS: 6763-6767 |
| IFNW1 | Interferon, omega 1 | SEQ ID NO: 6768 |
| IGF1 | Insulin-like growth factor 1 (somatomedin C) | SEQ ID NOS: 6769-6774 |
| IGF2 | Insulin-like growth factor 2 | SEQ ID NOS: 6775-6782 |
| IGFALS | Insulin-like growth factor binding protein, acid labile | SEQ ID NOS: 6783-6785 |
| | subunit | |
| IGFBP1 | Insulin-like growth factor binding protein 1 | SEQ ID NOS: 6786-6788 |
| IGFBP2 | Insulin-like growth factor binding protein 2, 36 kDa | SEQ ID NOS: 6789-6792 |
| IGFBP3 | Insulin-like growth factor binding protein 3 | SEQ ID NOS: 6793-6800 |
| IGFBP4 | Insulin-like growth factor binding protein 4 | SEQ ID NO: 6801 |
| IGFBP5 | Insulin-like growth factor binding protein 5 | SEQ ID NOS: 6802-6803 |
| IGFBP6 | Insulin-like growth factor binding protein 6 | SEQ ID NOS: 6804-6806 |
| IGFBP7 | Insulin-like growth factor binding protein 7 | SEQ ID NOS: 6807-6808 |
| IGFBPL1 | Insulin-like growth factor binding protein-like 1 | SEQ ID NO: 6809 |
| IGFL1 | IGF-like family member 1 | SEQ ID NO: 6810 |
| IGFL2 | IGF-like family member 2 | SEQ ID NOS: 6811-6813 |
| IGFL3 | IGF-like family member 3 | SEQ ID NO: 6814 |
| IGFLR1 | IGF-like family receptor 1 | SEQ ID NOS: 6815-6823 |
| IGIP | IgA-inducing protein | SEQ ID NO: 6824 |
| IGLON5 | IgLON family member 5 | SEQ ID NO: 6825 |
| IGSF1 | Immunoglobulin superfamily, member 1 | SEQ ID NOS: 6826-6831 |
| IGSF10 | Immunoglobulin superfamily, member 10 | SEQ ID NOS: 6832-6833 |
| IGSF11 | Immunoglobulin superfamily, member 11 | SEQ ID NOS: 6834-6841 |
| IGSF21 | Immunoglobin superfamily, member 21 | SEQ ID NO: 6842 |
| IGSF8 | Immunoglobulin superfamily, member 8 | SEQ ID NOS: 6843-6846 |
| IGSF9 | Immunoglobulin superfamily, member 9 | SEQ ID NOS: 6847-6849 |
| IHH | Indian hedgehog | SEQ ID NO: 6850 |
| IL10 | Interleukin 10 | SEQ ID NOS: 6851-6852 |
| IL11 | Interleukin 11 | SEQ ID NOS: 6853-6856 |
| IL11RA | Interleukin 11 receptor, alpha | SEQ ID NOS: 6857-6867 |
| IL12B | Interleukin 12B | SEQ ID NO: 6868 |
| IL12RB1 | Interleukin 12 receptor, beta 1 | SEQ ID NOS: 6869-6874 |
| IL12RB2 | Interleukin 12 receptor, beta 2 | SEQ ID NOS: 6875-6879 |
| IL13 | Interleukin 13 | SEQ ID NOS: 6880-6881 |
| IL13RA1 | Interleukin 13 receptor, alpha 1 | SEQ ID NOS: 6882-6883 |
| IL15RA | Interleukin 15 receptor, alpha | SEQ ID NOS: 6884-6901 |
| IL17A | Interleukin 17A | SEQ ID NO: 6902 |
| IL17B | Interleukin 17B | SEQ ID NO: 6903 |
| IL17C | Interleukin 17C | SEQ ID NO: 6904 |
| IL17D | Interleukin 17D | SEQ ID NOS: 6905-6907 |
| IL17F | Interleukin 17F | SEQ ID NO: 6908 |
| IL17RA | Interleukin 17 receptor A | SEQ ID NOS: 6909-6910 |
| IL17RC | Interleukin 17 receptor C | SEQ ID NOS: 6911-6926 |
| IL17RE | Interleukin 17 receptor E | SEQ ID NOS: 6927-6933 |
| IL18BP | Interleukin 18 binding protein | SEQ ID NOS: 6934-6944 |
| IL18R1 | Interleukin 18 receptor 1 | SEQ ID NOS: 6945-6948 |
| IL18RAP | Interleukin 18 receptor accessory protein | SEQ ID NOS: 6949-6951 |
| IL19 | Interleukin 19 | SEQ ID NOS: 6952-6954 |
| IL1R1 | Interleukin 1 receptor, type I | SEQ ID NOS: 6955-6967 |
| IL1R2 | Interleukin 1 receptor, type II | SEQ ID NOS: 6968-6971 |
| IL1RAP | Interleukin 1 receptor accessory protein | SEQ ID NOS: 6972-6985 |
| IL1RL1 | Interleukin 1 receptor-like 1 | SEQ ID NOS: 6986-6991 |
| IL1RL2 | Interleukin 1 receptor-like 2 | SEQ ID NOS: 6992-6994 |
| IL1RN | Interleukin 1 receptor antagonist | SEQ ID NOS: 6995-6999 |
| IL2 | Interleukin 2 | SEQ ID NO: 7000 |
| IL20 | Interleukin 20 | SEQ ID NOS: 7001-7003 |
| IL20RA | Interleukin 20 receptor, alpha | SEQ ID NOS: 7004-7010 |
| IL21 | Interleukin 21 | SEQ ID NOS: 7011-7012 |
| IL22 | Interleukin 22 | SEQ ID NOS: 7013-7014 |
| IL22RA2 | Interleukin 22 receptor, alpha 2 | SEQ ID NOS: 7015-7017 |
| IL23A | Interleukin 23, alpha subunit p19 | SEQ ID NO: 7018 |
| IL24 | Interleukin 24 | SEQ ID NOS: 7019-7024 |
| IL25 | Interleukin 25 | SEQ ID NOS: 7025-7026 |
| IL26 | Interleukin 26 | SEQ ID NO: 7027 |
| IL27 | Interleukin 27 | SEQ ID NOS: 7028-7029 |
| IL2RB | Interleukin 2 receptor, beta | SEQ ID NOS: 7030-7034 |
| IL3 | Interleukin 3 | SEQ ID NO: 7035 |
| IL31 | Interleukin 31 | SEQ ID NO: 7036 |
| IL31RA | Interleukin 31 receptor A | SEQ ID NOS: 7037-7044 |
| IL32 | Interleukin 32 | SEQ ID NOS: 7045-7074 |
| IL34 | Interleukin 34 | SEQ ID NOS: 7075-7078 |
| IL3RA | Interleukin 3 receptor, alpha (low affinity) | SEQ ID NOS: 7079-7081 |
| IL4 | Interleukin 4 | SEQ ID NOS: 7082-7084 |
| IL4I1 | Interleukin 4 induced 1 | SEQ ID NOS: 7085-7092 |
| IL4R | Interleukin 4 receptor | SEQ ID NOS: 7093-7106 |
| IL5 | Interleukin 5 | SEQ ID NOS: 7107-7108 |
| IL5RA | Interleukin 5 receptor, alpha | SEQ ID NOS: 7109-7118 |
| IL6 | Interleukin 6 | SEQ ID NOS: 7119-7125 |
| IL6R | Interleukin 6 receptor | SEQ ID NOS: 7126-7131 |
| IL6ST | Interleukin 6 signal transducer | SEQ ID NOS: 7132-7141 |
| IL7 | Interleukin 7 | SEQ ID NOS: 7142-7149 |
| IL7R | Interleukin 7 receptor | SEQ ID NOS: 7150-7156 |
| IL9 | Interleukin 9 | SEQ ID NO: 7157 |
| ILDR1 | Immunoglobulin-like domain containing receptor 1 | SEQ ID NOS: 7158-7162 |
| ILDR2 | Immunoglobulin-like domain containing receptor 2 | SEQ ID NOS: 7163-7169 |
| IMP4 | IMP4, U3 small nucleolar ribonucleoprotein | SEQ ID NOS: 7170-7175 |
| IMPG1 | Interphotoreceptor matrix proteoglycan 1 | SEQ ID NOS: 7176-7179 |
| INHA | Inhibin, alpha | SEQ ID NO: 7180 |
| INHBA | Inhibin, beta A | SEQ ID NOS: 7181-7183 |
| INHBB | Inhibin, beta B | SEQ ID NO: 7184 |
| INHBC | Inhibin, beta C | SEQ ID NO: 7185 |
| INHBE | Inhibin, beta E | SEQ ID NOS: 7186-7187 |
| INPP5A | Inositol polyphosphate-5-phosphatase A | SEQ ID NOS: 7188-7192 |
| INS | Insulin | SEQ ID NOS: 7193-7197 |
| INS-IGF2 | INS-IGF2 readthrough | SEQ ID NOS: 7198-7199 |
| INSL3 | Insulin-like 3 (Leydig cell) | SEQ ID NOS: 7200-7202 |
| INSL4 | Insulin-like 4 (placenta) | SEQ ID NO: 7203 |
| INSL5 | Insulin-like 5 | SEQ ID NO: 7204 |
| INSL6 | Insulin-like 6 | SEQ ID NO: 7205 |
| INTS3 | Integrator complex subunit 3 | SEQ ID NOS: 7206-7211 |
| IPO11 | Importin 11 | SEQ ID NOS: 7212-7220 |
| IPO9 | Importin 9 | SEQ ID NOS: 7221-7222 |
| IQCF6 | IQ motif containing F6 | SEQ ID NOS: 7223-7224 |
| IRAK3 | Interleukin-1 receptor-associated kinase 3 | SEQ ID NOS: 7225-7227 |
| IRS4 | Insulin receptor substrate 4 | SEQ ID NO: 7228 |
| ISLR | Immunoglobulin superfamily containing leucine-rich | SEQ ID NOS: 7229-7232 |
| | repeat | |
| ISLR2 | Immunoglobulin superfamily containing leucine-rich | SEQ ID NOS: 7233-7242 |
| | repeat 2 | |
| ISM1 | Isthmin 1, angiogenesis inhibitor | SEQ ID NO: 7243 |
| ISM2 | Isthmin 2 | SEQ ID NOS: 7244-7249 |
| ITGA4 | Integrin, alpha 4 (antigen CD49D, alpha 4 subunit of | SEQ ID NOS: 7250-7252 |
| | VLA-4 receptor) | |
| ITGA9 | Integrin, alpha 9 | SEQ ID NOS: 7253-7255 |
| ITGAL | Integrin, alpha L (antigen CD11A (p180), lymphocyte | SEQ ID NOS: 7256-7265 |
| | function-associated antigen 1; alpha polypeptide) | |
| ITGAX | Integrin, alpha X (complement component 3 | SEQ ID NOS: 7266-7268 |
| | receptor 4 subunit) | |
| ITGB1 | Integrin, beta 1 (fibronectin receptor, beta | SEQ ID NOS: 7269-7284 |
| | polypeptide, antigen CD29 includes MDF2, MSK12) | |
| ITGB2 | Integrin, beta 2 (complement component 3 receptor | SEQ ID NOS: 7285-7301 |
| | 3 and 4 subunit) | |
| ITGB3 | Integrin, beta 3 (platelet glycoprotein IIIa, antigen | SEQ ID NOS: 7302-7304 |
| | CD61) | |
| ITGB7 | Integrin, beta 7 | SEQ ID NOS: 7305-7312 |
| ITGBL1 | Integrin, beta-like 1 (with EGF-like repeat domains) | SEQ ID NOS: 7313-7318 |
| ITIH1 | Inter-alpha-trypsin inhibitor heavy chain 1 | SEQ ID NOS: 7319-7324 |
| ITIH2 | Inter-alpha-trypsin inhibitor heavy chain 2 | SEQ ID NOS: 7325-7327 |
| ITIH3 | Inter-alpha-trypsin inhibitor heavy chain 3 | SEQ ID NOS: 7328-7330 |
| ITIH4 | Inter-alpha-trypsin inhibitor heavy chain family, | SEQ ID NOS: 7331-7334 |
| | member 4 | |
| ITIH5 | Inter-alpha-trypsin inhibitor heavy chain family, | SEQ ID NOS: 7335-7338 |
| | member 5 | |
| ITIH6 | Inter-alpha-trypsin inhibitor heavy chain family, | SEQ ID NO: 7339 |
| | member 6 | |
| ITLN1 | Intelectin 1 (galactofuranose binding) | SEQ ID NO: 7340 |
| ITLN2 | Intelectin 2 | SEQ ID NO: 7341 |
| IZUMO1R | IZUMO1 receptor, JUNO | SEQ ID NOS: 7342-7343 |
| IZUMO4 | IZUMO family member 4 | SEQ ID NOS: 7344-7350 |
| AMICA1 | Adhesion molecule, interacts with CXADR antigen 1 | SEQ ID NOS: 7351-7359 |
| JCHAIN | Joining chain of multimeric IgA and IgM | SEQ ID NOS: 7360-7365 |
| JMJD8 | Jumonji domain containing 8 | SEQ ID NOS: 7366-7370 |
| JSRP1 | Junctional sarcoplasmic reticulum protein 1 | SEQ ID NO: 7371 |
| KANSL2 | KAT8 regulatory NSL complex subunit 2 | SEQ ID NOS: 7372-7382 |
| KAZALD1 | Kazal-type serine peptidase inhibitor domain 1 | SEQ ID NO: 7383 |
| KCNIP3 | Kv channel interacting protein 3, calsenilin | SEQ ID NOS: 7384-7386 |
| KCNK7 | Potassium channel, two pore domain subfamily K, | SEQ ID NOS: 7387-7392 |
| | member 7 | |
| KCNN4 | Potassium channel, calcium activated | SEQ ID NOS: 7393-7398 |
| | intermediate/small conductance subfamily N alpha, | |
| | member 4 | |
| KCNU1 | Potassium channel, subfamily U, member 1 | SEQ ID NOS: 7399-7403 |
| KCP | Kielin/chordin-like protein | SEQ ID NOS: 7404-7407 |
| KDELC1 | KDEL (Lys-Asp-Glu-Leu) containing 1 | SEQ ID NO: 7408 |
| KDELC2 | KDEL (Lys-Asp-Glu-Leu) containing 2 | SEQ ID NOS: 7409-7412 |
| KDM1A | Lysine (K)-specific demethylase 1A | SEQ ID NOS: 7413-7416 |
| KDM3B | Lysine (K)-specific demethylase 3B | SEQ ID NOS: 7417-7420 |
| KDM6A | Lysine (K)-specific demethylase 6A | SEQ ID NOS: 7421-7430 |
| KDM7A | Lysine (K)-specific demethylase 7A | SEQ ID NOS: 7431-7432 |
| KDSR | 3-ketodihydrosphingosine reductase | SEQ ID NOS: 7433-7439 |
| KERA | Keratocan | SEQ ID NO: 7440 |
| KIAA0100 | KIAA0100 | SEQ ID NOS: 7441-7446 |
| KIAA0319 | KIAA0319 | SEQ ID NOS: 7447-7452 |
| KIAA1324 | KIAA1324 | SEQ ID NOS: 7453-7461 |
| KIFC2 | Kinesin family member C2 | SEQ ID NOS: 7462-7464 |
| KIR2DL4 | Killer cell immunoglobulin-like receptor, two | SEQ ID NOS: 7465-7471 |
| | domains, long cytoplasmic tail, 4 | |
| KIR3DX1 | Killer cell immunoglobulin-like receptor, three | SEQ ID NOS: 7472-7476 |
| | domains, X1 | |
| KIRREL2 | Kin of IRRE like 2 (Drosophila) | SEQ ID NOS: 7477-7481 |
| KISS1 | KiSS-1 metastasis-suppressor | SEQ ID NOS: 7482-7483 |
| KLHL11 | Kelch-like family member 11 | SEQ ID NO: 7484 |
| KLHL22 | Kelch-like family member 22 | SEQ ID NOS: 7485-7491 |
| KLK1 | Kallikrein 1 | SEQ ID NOS: 7492-7493 |
| KLK10 | Kallikrein-related peptidase 10 | SEQ ID NOS: 7494-7498 |
| KLK11 | Kallikrein-related peptidase 11 | SEQ ID NOS: 7499-7507 |
| KLK12 | Kallikrein-related peptidase 12 | SEQ ID NOS: 7508-7514 |
| KLK13 | Kallikrein-related peptidase 13 | SEQ ID NOS: 7515-7523 |
| KLK14 | Kallikrein-related peptidase 14 | SEQ ID NOS: 7524-7525 |
| KLK15 | Kallikrein-related peptidase 15 | SEQ ID NOS: 7526-7530 |
| KLK2 | Kallikrein-related peptidase 2 | SEQ ID NOS: 7531-7543 |
| KLK3 | Kallikrein-related peptidase 3 | SEQ ID NOS: 7544-7555 |
| KLK4 | Kallikrein-related peptidase 4 | SEQ ID NOS: 7556-7560 |
| KLK5 | Kallikrein-related peptidase 5 | SEQ ID NOS: 7561-7564 |
| KLK6 | Kallikrein-related peptidase 6 | SEQ ID NOS: 7565-7571 |
| KLK7 | Kallikrein-related peptidase 7 | SEQ ID NOS: 7572-7576 |
| KLK8 | Kallikrein-related peptidase 8 | SEQ ID NOS: 7577-7584 |
| KLK9 | Kallikrein-related peptidase 9 | SEQ ID NOS: 7585-7586 |
| KLKB1 | Kallikrein B, plasma (Fletcher factor) 1 | SEQ ID NOS: 7587-7591 |
| SETD8 | SET domain containing (lysine methyltransferase) 8 | SEQ ID NOS: 7592-7595 |
| KNDC1 | Kinase non-catalytic C-lobe domain (KIND) | SEQ ID NOS: 7596-7597 |
| | containing 1 | |
| KNG1 | Kininogen 1 | SEQ ID NOS: 7598-7602 |
| KRBA2 | KRAB-A domain containing 2 | SEQ ID NOS: 7603-7606 |
| KREMEN2 | Kringle containing transmembrane protein 2 | SEQ ID NOS: 7607-7612 |
| KRTDAP | Keratinocyte differentiation-associated protein | SEQ ID NOS: 7613-7614 |
| L1CAM | L1 cell adhesion molecule | SEQ ID NOS: 7615-7624 |
| L3MBTL2 | L(3)mbt-like 2 (Drosophila) | SEQ ID NOS: 7625-7629 |
| LACRT | Lacritin | SEQ ID NOS: 7630-7632 |
| LACTB | Lactamase, beta | SEQ ID NOS: 7633-7635 |
| LAG3 | Lymphocyte-activation gene 3 | SEQ ID NOS: 7636-7637 |
| LAIR2 | Leukocyte-associated immunoglobulin-like | SEQ ID NOS: 7638-7641 |
| | receptor 2 | |
| LALBA | Lactalbumin, alpha- | SEQ ID NOS: 7642-7643 |
| LAMA1 | Laminin, alpha 1 | SEQ ID NOS: 7644-7645 |
| LAMA2 | Laminin, alpha 2 | SEQ ID NOS: 7646-7649 |
| LAMA3 | Laminin, alpha 3 | SEQ ID NOS: 7650-7659 |
| LAMA4 | Laminin, alpha 4 | SEQ ID NOS: 7660-7674 |
| LAMAS | Laminin, alpha 5 | SEQ ID NOS: 7675-7677 |
| LAMB1 | Laminin, beta 1 | SEQ ID NOS: 7678-7682 |
| LAMB2 | Laminin, beta 2 (laminin S) | SEQ ID NOS: 7683-7685 |
| LAMB3 | Laminin, beta 3 | SEQ ID NOS: 7686-7690 |
| LAMB4 | Laminin, beta 4 | SEQ ID NOS: 7691-7694 |
| LAMC1 | Laminin, gamma 1 (formerly LAMB2) | SEQ ID NOS: 7695-7696 |
| LAMC2 | Laminin, gamma 2 | SEQ ID NOS: 7697-7698 |
| LAMC3 | Laminin, gamma 3 | SEQ ID NOS: 7699-7700 |
| LAMP3 | Lysosomal-associated membrane protein 3 | SEQ ID NOS: 7701-7704 |
| GYLTL1B | Glycosyltransferase-like 1B | SEQ ID NOS: 7705-7710 |
| LAT | Linker for activation of T cells | SEQ ID NOS: 7711-7720 |
| LAT2 | Linker for activation of T cells family, member 2 | SEQ ID NOS: 7721-7729 |
| LBP | Lipopolysaccharide binding protein | SEQ ID NO: 7730 |
| LCAT | Lecithin-cholesterol acyltransferase | SEQ ID NOS: 7731-7737 |
| LCN1 | Lipocalin 1 | SEQ ID NOS: 7738-7739 |
| LCN10 | Lipocalin 10 | SEQ ID NOS: 7740-7745 |
| LCN12 | Lipocalin 12 | SEQ ID NOS: 7746-7748 |
| LCN15 | Lipocalin 15 | SEQ ID NO: 7749 |
| LCN2 | Lipocalin 2 | SEQ ID NOS: 7750-7752 |
| LCN6 | Lipocalin 6 | SEQ ID NOS: 7753-7754 |
| LCN8 | Lipocalin 8 | SEQ ID NOS: 7755-7756 |
| LCN9 | Lipocalin 9 | SEQ ID NOS: 7757-7758 |
| LCORL | Ligand dependent nuclear receptor corepressor-like | SEQ ID NOS: 7759-7764 |
| LDLR | Low density lipoprotein receptor | SEQ ID NOS: 7765-7773 |
| LDLRAD2 | Low density lipoprotein receptor class A domain | SEQ ID NOS: 7774-7775 |
| | containing 2 | |
| LEAP2 | Liver expressed antimicrobial peptide 2 | SEQ ID NO: 7776 |
| LECT2 | Leukocyte cell-derived chemotaxin 2 | SEQ ID NOS: 7777-7780 |
| LEFTY1 | Left-right determination factor 1 | SEQ ID NOS: 7781-7782 |
| LEFTY2 | Left-right determination factor 2 | SEQ ID NOS: 7783-7784 |
| LEP | Leptin | SEQ ID NO: 7785 |
| LFNG | LFNG O-fucosylpeptide 3-beta-N- | SEQ ID NOS: 7786-7791 |
| | acetylglucosaminyltransferase | |
| LGALS3BP | Lectin, galactoside-binding, soluble, 3 binding | SEQ ID NOS: 7792-7806 |
| | protein | |
| LGI1 | Leucine-rich, glioma inactivated 1 | SEQ ID NOS: 7807-7825 |
| LGI2 | Leucine-rich repeat LGI family, member 2 | SEQ ID NOS: 7826-7827 |
| LGI3 | Leucine-rich repeat LGI family, member 3 | SEQ ID NOS: 7828-7831 |
| LGI4 | Leucine-rich repeat LGI family, member 4 | SEQ ID NOS: 7832-7835 |
| LGMN | Legumain | SEQ ID NOS: 7836-7849 |
| LGR4 | Leucine-rich repeat containing G protein-coupled | SEQ ID NOS: 7850-7852 |
| | receptor 4 | |
| LHB | Luteinizing hormone beta polypeptide | SEQ ID NO: 7853 |
| LHCGR | Luteinizing hormone/choriogonadotropin receptor | SEQ ID NOS: 7854-7858 |
| LIF | Leukemia inhibitory factor | SEQ ID NOS: 7859-7860 |
| LIFR | Leukemia inhibitory factor receptor alpha | SEQ ID NOS: 7861-7865 |
| LILRA1 | Leukocyte immunoglobulin-like receptor, subfamily | SEQ ID NOS: 7866-7867 |
| | A (with TM domain), member 1 | |
| LILRA2 | Leukocyte immunoglobulin-like receptor, subfamily | SEQ ID NOS: 7868-7874 |
| | A (with TM domain), member 2 | |
| LILRB3 | Leukocyte immunoglobulin-like receptor, subfamily | SEQ ID NOS: 7875-7879 |
| | B (with TM and ITIM domains), member 3 | |
| LIME1 | Lck interacting transmembrane adaptor 1 | SEQ ID NOS: 7880-7885 |
| LINGO1 | Leucine rich repeat and Ig domain containing 1 | SEQ ID NOS: 7886-7896 |
| LIPA | Lipase A, lysosomal acid, cholesterol esterase | SEQ ID NOS: 7897-7901 |
| LIPC | Lipase, hepatic | SEQ ID NOS: 7902-7905 |
| LIPF | Lipase, gastric | SEQ ID NOS: 7906-7909 |
| LIPG | Lipase, endothelial | SEQ ID NOS: 7910-7915 |
| LIPH | Lipase, member H | SEQ ID NOS: 7916-7920 |
| LIPK | Lipase, family member K | SEQ ID NO: 7921 |
| LIPM | Lipase, family member M | SEQ ID NOS: 7922-7923 |
| LIPN | Lipase, family member N | SEQ ID NO: 7924 |
| LMAN2 | Lectin, mannose-binding 2 | SEQ ID NOS: 7925-7929 |
| LMNTD1 | Lamin tail domain containing 1 | SEQ ID NOS: 7930-7940 |
| LNX1 | Ligand of numb-protein X 1, E3 ubiquitin protein | SEQ ID NOS: 7941-7947 |
| | ligase | |
| LOX | Lysyl oxidase | SEQ ID NOS: 7948-7950 |
| LOXL1 | Lysyl oxidase-like 1 | SEQ ID NOS: 7951-7952 |
| LOXL2 | Lysyl oxidase-like 2 | SEQ ID NOS: 7953-7961 |
| LOXL3 | Lysyl oxidase-like 3 | SEQ ID NOS: 7962-7968 |
| LOXL4 | Lysyl oxidase-like 4 | SEQ ID NO: 7969 |
| LPA | Lipoprotein, Lp(a) | SEQ ID NOS: 7970-7972 |
| LPL | Lipoprotein lipase | SEQ ID NOS: 7973-7977 |
| LPO | Lactoperoxidase | SEQ ID NOS: 7978-7984 |
| LRAT | Lecithin retinol acyltransferase | SEQ ID NOS: 7985-7987 |
| | (phosphatidylcholine-retinol O-acyltransferase) | |
| LRCH3 | Leucine-rich repeats and calponin homology (CH) | SEQ ID NOS: 7988-7996 |
| | domain containing 3 | |
| LRCOL1 | Leucine rich colipase-like 1 | SEQ ID NOS: 7997-8000 |
| LRFN4 | Leucine rich repeat and fibronectin type III domain | SEQ ID NOS: 8001-8002 |
| | containing 4 | |
| LRFN5 | Leucine rich repeat and fibronectin type III domain | SEQ ID NOS: 8003-8005 |
| | containing 5 | |
| LRG1 | Leucine-rich alpha-2-glycoprotein 1 | SEQ ID NO: 8006 |
| LRP1 | Low density lipoprotein receptor-related protein 1 | SEQ ID NOS: 8007-8012 |
| LRP11 | Low density lipoprotein receptor-related protein 11 | SEQ ID NOS: 8013-8014 |
| LRP1B | Low density lipoprotein receptor-related protein 1B | SEQ ID NOS: 8015-8018 |
| LRP2 | Low density lipoprotein receptor-related protein 2 | SEQ ID NOS: 8019-8020 |
| LRP4 | Low density lipoprotein receptor-related protein 4 | SEQ ID NOS: 8021-8022 |
| LRPAP1 | Low density lipoprotein receptor-related protein | SEQ ID NOS: 8023-8024 |
| | associated protein 1 | |
| LRRC17 | Leucine rich repeat containing 17 | SEQ ID NOS: 8025-8027 |
| LRRC32 | Leucine rich repeat containing 32 | SEQ ID NOS: 8028-8031 |
| LRRC3B | Leucine rich repeat containing 3B | SEQ ID NOS: 8032-8036 |
| LRRC4B | Leucine rich repeat containing 4B | SEQ ID NOS: 8037-8039 |
| LRRC70 | Leucine rich repeat containing 70 | SEQ ID NOS: 8040-8041 |
| LRRN3 | Leucine rich repeat neuronal 3 | SEQ ID NOS: 8042-8045 |
| LRRTM1 | Leucine rich repeat transmembrane neuronal 1 | SEQ ID NOS: 8046-8052 |
| LRRTM2 | Leucine rich repeat transmembrane neuronal 2 | SEQ ID NOS: 8053-8055 |
| LRRTM4 | Leucine rich repeat transmembrane neuronal 4 | SEQ ID NOS: 8056-8061 |
| LRTM2 | Leucine-rich repeats and transmembrane domains 2 | SEQ ID NOS: 8062-8066 |
| LSR | Lipolysis stimulated lipoprotein receptor | SEQ ID NOS: 8067-8077 |
| LST1 | Leukocyte specific transcript 1 | SEQ ID NOS: 8078-8095 |
| LTA | Lymphotoxin alpha | SEQ ID NOS: 8096-8097 |
| LTBP1 | Latent transforming growth factor beta binding | SEQ ID NOS: 8098-8107 |
| | protein 1 | |
| LTBP2 | Latent transforming growth factor beta binding | SEQ ID NOS: 8108-8111 |
| | protein 2 | |
| LTBP3 | Latent transforming growth factor beta binding | SEQ ID NOS: 8112-8124 |
| | protein 3 | |
| LTBP4 | Latent transforming growth factor beta binding | SEQ ID NOS: 8125-8140 |
| | protein 4 | |
| LTBR | Lymphotoxin beta receptor (TNFR superfamily, | SEQ ID NOS: 8141-8146 |
| | member 3) | |
| LTF | Lactotransferrin | SEQ ID NOS: 8147-8151 |
| LTK | Leukocyte receptor tyrosine kinase | SEQ ID NOS: 8152-8155 |
| LUM | Lumican | SEQ ID NO: 8156 |
| LUZP2 | Leucine zipper protein 2 | SEQ ID NOS: 8157-8160 |
| LVRN | Laeverin | SEQ ID NOS: 8161-8166 |
| LY6E | Lymphocyte antigen 6 complex, locus E | SEQ ID NOS: 8167-8180 |
| LY6G5B | Lymphocyte antigen 6 complex, locus G5B | SEQ ID NOS: 8181-8182 |
| LY6G6D | Lymphocyte antigen 6 complex, locus G6D | SEQ ID NOS: 8183-8184 |
| LY6G6E | Lymphocyte antigen 6 complex, locus G6E | SEQ ID NOS: 8185-8188 |
| | (pseudogene) | |
| LY6H | Lymphocyte antigen 6 complex, locus H | SEQ ID NOS: 8189-8192 |
| LY6K | Lymphocyte antigen 6 complex, locus K | SEQ ID NOS: 8193-8196 |
| RP11- | | SEQ ID NO: 8197 |
| 520P18.5 | | |
| LY86 | Lymphocyte antigen 86 | SEQ ID NOS: 8198-8199 |
| LY96 | Lymphocyte antigen 96 | SEQ ID NOS: 8200-8201 |
| LYG1 | Lysozyme G-like 1 | SEQ ID NOS: 8202-8203 |
| LYG2 | Lysozyme G-like 2 | SEQ ID NOS: 8204-8209 |
| LYNX1 | Ly6/neurotoxin 1 | SEQ ID NOS: 8210-8214 |
| LYPD1 | LY6/PLAUR domain containing 1 | SEQ ID NOS: 8215-8217 |
| LYPD2 | LY6/PLAUR domain containing 2 | SEQ ID NO: 8218 |
| LYPD4 | LY6/PLAUR domain containing 4 | SEQ ID NOS: 8219-8221 |
| LYPD6 | LY6/PLAUR domain containing 6 | SEQ ID NOS: 8222-8226 |
| LYPD6B | LY6/PLAUR domain containing 6B | SEQ ID NOS: 8227-8233 |
| LYPD8 | LY6/PLAUR domain containing 8 | SEQ ID NOS: 8234-8235 |
| LYZ | Lysozyme | SEQ ID NOS: 8236-8238 |
| LYZL4 | Lysozyme-like 4 | SEQ ID NOS: 8239-8240 |
| LYZL6 | Lysozyme-like 6 | SEQ ID NOS: 8241-8243 |
| M6PR | Mannose-6-phosphate receptor (cation dependent) | SEQ ID NOS: 8244-8254 |
| MAD1L1 | MAD1 mitotic arrest deficient-like 1 (yeast) | SEQ ID NOS: 8255-8267 |
| MAG | Myelin associated glycoprotein | SEQ ID NOS: 8268-8273 |
| MAGT1 | Magnesium transporter 1 | SEQ ID NOS: 8274-8277 |
| MALSU1 | Mitochondrial assembly of ribosomal large subunit 1 | SEQ ID NO: 8278 |
| MAMDC2 | MAM domain containing 2 | SEQ ID NO: 8279 |
| MAN2B1 | Mannosidase, alpha, class 2B, member 1 | SEQ ID NOS: 8280-8285 |
| MAN2B2 | Mannosidase, alpha, class 2B, member 2 | SEQ ID NOS: 8286-8288 |
| MANBA | Mannosidase, beta A, lysosomal | SEQ ID NOS: 8289-8302 |
| MANEAL | Mannosidase, endo-alpha-like | SEQ ID NOS: 8303-8307 |
| MANF | Mesencephalic astrocyte-derived neurotrophic | SEQ ID NOS: 8308-8309 |
| | factor | |
| MANSC1 | MANSC domain containing 1 | SEQ ID NOS: 8310-8313 |
| MAP3K9 | Mitogen-activated protein kinase 9 | SEQ ID NOS: 8314-8319 |
| MASP1 | Mannan-binding lectin serine peptidase 1 (C4/C2 | SEQ ID NOS: 8320-8327 |
| | activating component of Ra-reactive factor) | |
| MASP2 | Mannan-binding lectin serine peptidase 2 | SEQ ID NOS: 8328-8329 |
| MATN1 | Matrilin 1, cartilage matrix protein | SEQ ID NO: 8330 |
| MATN2 | Matrilin 2 | SEQ ID NOS: 8331-8343 |
| MATN3 | Matrilin 3 | SEQ ID NOS: 8344-8345 |
| MATN4 | Matrilin 4 | SEQ ID NOS: 8346-8350 |
| MATR3 | Matrin 3 | SEQ ID NOS: 8351-8378 |
| MAU2 | MAU2 sister chromatid cohesion factor | SEQ ID NOS: 8379-8381 |
| MAZ | MYC-associated zinc finger protein (purine-binding | SEQ ID NOS: 8382-8396 |
| | transcription factor) | |
| MBD6 | Methyl-CpG binding domain protein 6 | SEQ ID NOS: 8397-8408 |
| MBL2 | Mannose-binding lectin (protein C) 2, soluble | SEQ ID NO: 8409 |
| MBNL1 | Muscleblind-like splicing regulator 1 | SEQ ID NOS: 8410-8428 |
| MCCC1 | Methylcrotonoyl-CoA carboxylase 1 (alpha) | SEQ ID NOS: 8429-8440 |
| MCCD1 | Mitochondrial coiled-coil domain 1 | SEQ ID NO: 8441 |
| MCEE | Methylmalonyl CoA epimerase | SEQ ID NOS: 8442-8445 |
| MCF2L | MCF.2 cell line derived transforming sequence-like | SEQ ID NOS: 8446-8467 |
| MCFD2 | Multiple coagulation factor deficiency 2 | SEQ ID NOS: 8468-8479 |
| MDFIC | MyoD family inhibitor domain containing | SEQ ID NOS: 8480-8487 |
| MDGA1 | MAM domain containing | SEQ ID NOS: 8488-8493 |
| | glycosylphosphatidylinositol anchor 1 | |
| MDK | Midkine (neurite growth-promoting factor 2) | SEQ ID NOS: 8494-8503 |
| MED20 | Mediator complex subunit 20 | SEQ ID NOS: 8504-8508 |
| MEGF10 | Multiple EGF-like-domains 10 | SEQ ID NOS: 8509-8512 |
| MEGF6 | Multiple EGF-like-domains 6 | SEQ ID NOS: 8513-8516 |
| MEI1 | Meiotic double-stranded break formation protein 1 | SEQ ID NOS: 8517-8520 |
| MEI4 | Meiotic double-stranded break formation protein 4 | SEQ ID NO: 8521 |
| MEIS1 | Meis homeobox 1 | SEQ ID NOS: 8522-8527 |
| MEIS3 | Meis homeobox 3 | SEQ ID NOS: 8528-8537 |
| MFI2 | Antigen p97 (melanoma associated) identified by | SEQ ID NOS: 8538-8540 |
| | monoclonal antibodies 133.2 and 96.5 | |
| MEPE | Matrix extracellular phosphoglycoprotein | SEQ ID NOS: 8541-8547 |
| MESDC2 | Mesoderm development candidate 2 | SEQ ID NOS: 8548-8552 |
| MEST | Mesoderm specific transcript | SEQ ID NOS: 8553-8566 |
| MET | MET proto-oncogene, receptor tyrosine kinase | SEQ ID NOS: 8567-8572 |
| METRN | Meteorin, glial cell differentiation regulator | SEQ ID NOS: 8573-8577 |
| METRNL | Meteorin, glial cell differentiation regulator-like | SEQ ID NOS: 8578-8581 |
| METTL17 | Methyltransferase like 17 | SEQ ID NOS: 8582-8592 |
| METTL24 | Methyltransferase like 24 | SEQ ID NO: 8593 |
| METTL7B | Methyltransferase like 7B | SEQ ID NOS: 8594-8595 |
| METTL9 | Methyltransferase like 9 | SEQ ID NOS: 8596-8604 |
| MEX3C | Mex-3 RNA binding family member C | SEQ ID NOS: 8605-8607 |
| MFAP2 | Microfibrillar-associated protein 2 | SEQ ID NOS: 8608-8609 |
| MFAP3 | Microfibrillar-associated protein 3 | SEQ ID NOS: 8610-8614 |
| MFAP3L | Microfibrillar-associated protein 3-like | SEQ ID NOS: 8615-8624 |
| MFAP4 | Microfibrillar-associated protein 4 | SEQ ID NOS: 8625-8627 |
| MFAP5 | Microfibrillar associated protein 5 | SEQ ID NOS: 8628-8638 |
| MFGE8 | Milk fat globule-EGF factor 8 protein | SEQ ID NOS: 8639-8645 |
| MFNG | MFNG O-fucosylpeptide 3-beta-N- | SEQ ID NOS: 8646-8653 |
| | acetylglucosaminyltransferase | |
| MGA | MGA, MAX dimerization protein | SEQ ID NOS: 8654-8662 |
| MGAT2 | Mannosyl (alpha-1,6-)-glycoprotein beta-1,2-N- | SEQ ID NO: 8663 |
| | acetylglucosaminyltransferase | |
| MGAT3 | Mannosyl (beta-1,4-)-glycoprotein beta-1,4-N- | SEQ ID NOS: 8664-8666 |
| | acetylglucosaminyltransferase | |
| MGAT4A | Mannosyl (alpha-1,3-)-glycoprotein beta-1,4-N- | SEQ ID NOS: 8667-8671 |
| | acetylglucosaminyltransferase, isozyme A | |
| MGAT4B | Mannosyl (alpha-1,3-)-glycoprotein beta-1,4-N- | SEQ ID NOS: 8672-8682 |
| | acetylglucosaminyltransferase, isozyme B | |
| MGAT4D | MGAT4 family, member D | SEQ ID NOS: 8683-8688 |
| MGLL | Monoglyceride lipase | SEQ ID NOS: 8689-8698 |
| MGP | Matrix Gla protein | SEQ ID NOS: 8699-8701 |
| MGST2 | Microsomal glutathione S-transferase 2 | SEQ ID NOS: 8702-8705 |
| MIA | Melanoma inhibitory activity | SEQ ID NOS: 8706-8711 |
| MIA2 | Melanoma inhibitory activity 2 | SEQ ID NO: 8712 |
| MIA3 | Melanoma inhibitory activity family, member 3 | SEQ ID NOS: 8713-8717 |
| MICU1 | Mitochondrial calcium uptake 1 | SEQ ID NOS: 8718-8727 |
| MIER1 | Mesoderm induction early response 1, | SEQ ID NOS: 8728-8736 |
| | transcriptional regulator | |
| MINOS1- | MINOS1-NBL1 readthrough | SEQ ID NOS: 8737-8739 |
| NBL1 | | |
| MINPP1 | Multiple inositol-polyphosphate phosphatase 1 | SEQ ID NOS: 8740-8742 |
| MLEC | Malectin | SEQ ID NOS: 8743-8746 |
| MLN | Motilin | SEQ ID NOS: 8747-8749 |
| MLXIP | MLX interacting protein | SEQ ID NOS: 8750-8755 |
| MLXIPL | MLX interacting protein-like | SEQ ID NOS: 8756-8763 |
| MMP1 | Matrix metallopeptidase 1 | SEQ ID NO: 8764 |
| MMP10 | Matrix metallopeptidase 10 | SEQ ID NOS: 8765-8766 |
| MMP11 | Matrix metallopeptidase 11 | SEQ ID NOS: 8767-8770 |
| MMP12 | Matrix metallopeptidase 12 | SEQ ID NO: 8771 |
| MMP13 | Matrix metallopeptidase 13 | SEQ ID NOS: 8772-8774 |
| MMP14 | Matrix metallopeptidase 14 (membrane-inserted) | SEQ ID NOS: 8775-8777 |
| MMP17 | Matrix metallopeptidase 17 (membrane-inserted) | SEQ ID NOS: 8778-8785 |
| MMP19 | Matrix metallopeptidase 19 | SEQ ID NOS: 8786-8791 |
| MMP2 | Matrix metallopeptidase 2 | SEQ ID NOS: 8792-8799 |
| MMP20 | Matrix metallopeptidase 20 | SEQ ID NO: 8800 |
| MMP21 | Matrix metallopeptidase 21 | SEQ ID NO: 8801 |
| MMP25 | Matrix metallopeptidase 25 | SEQ ID NOS: 8802-8803 |
| MMP26 | Matrix metallopeptidase 26 | SEQ ID NOS: 8804-8805 |
| MMP27 | Matrix metallopeptidase 27 | SEQ ID NO: 8806 |
| MMP28 | Matrix metallopeptidase 28 | SEQ ID NOS: 8807-8812 |
| MMP3 | Matrix metallopeptidase 3 | SEQ ID NOS: 8813-8815 |
| MMP7 | Matrix metallopeptidase 7 | SEQ ID NO: 8816 |
| MMP8 | Matrix metallopeptidase 8 | SEQ ID NOS: 8817-8822 |
| MMP9 | Matrix metallopeptidase 9 | SEQ ID NO: 8823 |
| MMRN1 | Multimerin 1 | SEQ ID NOS: 8824-8826 |
| MMRN2 | Multimerin 2 | SEQ ID NOS: 8827-8831 |
| MOXD1 | Monooxygenase, DBH-like 1 | SEQ ID NOS: 8832-8834 |
| C6orf25 | Chromosome 6 open reading frame 25 | SEQ ID NOS: 8835-8842 |
| MPO | Myeloperoxidase | SEQ ID NOS: 8843-8844 |
| MPPED1 | Metallophosphoesterase domain containing 1 | SEQ ID NOS: 8845-8848 |
| MPZL1 | Myelin protein zero-like 1 | SEQ ID NOS: 8849-8853 |
| MR1 | Major histocompatibility complex, class I-related | SEQ ID NOS: 8854-8859 |
| MRPL2 | Mitochondrial ribosomal protein L2 | SEQ ID NOS: 8860-8864 |
| MRPL21 | Mitochondrial ribosomal protein L21 | SEQ ID NOS: 8865-8871 |
| MRPL22 | Mitochondrial ribosomal protein L22 | SEQ ID NOS: 8872-8876 |
| MRPL24 | Mitochondrial ribosomal protein L24 | SEQ ID NOS: 8877-8881 |
| MRPL27 | Mitochondrial ribosomal protein L27 | SEQ ID NOS: 8882-8887 |
| MRPL32 | Mitochondrial ribosomal protein L32 | SEQ ID NOS: 8888-8890 |
| MRPL34 | Mitochondrial ribosomal protein L34 | SEQ ID NOS: 8891-8895 |
| MRPL35 | Mitochondrial ribosomal protein L35 | SEQ ID NOS: 8896-8899 |
| MRPL52 | Mitochondrial ribosomal protein L52 | SEQ ID NOS: 8900-8910 |
| MRPL55 | Mitochondrial ribosomal protein L55 | SEQ ID NOS: 8911-8936 |
| MRPS14 | Mitochondrial ribosomal protein S14 | SEQ ID NOS: 8937-8938 |
| MRPS22 | Mitochondrial ribosomal protein S22 | SEQ ID NOS: 8939-8947 |
| MRPS28 | Mitochondrial ribosomal protein S28 | SEQ ID NOS: 8948-8955 |
| MS4A14 | Membrane-spanning 4-domains, subfamily A, | SEQ ID NOS: 8956-8966 |
| | member 14 | |
| MS4A3 | Membrane-spanning 4-domains, subfamily A, | SEQ ID NOS: 8967-8971 |
| | member 3 (hematopoietic cell-specific) | |
| MSH3 | MutS homolog 3 | SEQ ID NO: 8972 |
| MSH5 | MutS homolog 5 | SEQ ID NOS: 8973-8984 |
| MSLN | Mesothelin | SEQ ID NOS: 8985-8992 |
| MSMB | Microseminoprotein, beta- | SEQ ID NOS: 8993-8994 |
| MSRA | Methionine sulfoxide reductase A | SEQ ID NOS: 8995-9002 |
| MSRB2 | Methionine sulfoxide reductase B2 | SEQ ID NOS: 9003-9004 |
| MSRB3 | Methionine sulfoxide reductase B3 | SEQ ID NOS: 9005-9018 |
| MST1 | Macrophage stimulating 1 | SEQ ID NOS: 9019-9020 |
| MSTN | Myostatin | SEQ ID NO: 9021 |
| MT1G | Metallothionein 1G | SEQ ID NOS: 9022-9025 |
| MTHFD2 | Methylenetetrahydrofolate dehydrogenase (NADP + | SEQ ID NOS: 9026-9030 |
| | dependent) 2, methenyltetrahydrofolate | |
| | cyclohydrolase | |
| MTMR14 | Myotubularin related protein 14 | SEQ ID NOS: 9031-9041 |
| MTRNR2L11 | MT-RNR2-like 11 (pseudogene) | SEQ ID NO: 9042 |
| MTRR | 5-methyltetrahydrofolate-homocysteine | SEQ ID NOS: 9043-9055 |
| | methyltransferase reductase | |
| MTTP | Microsomal triglyceride transfer protein | SEQ ID NOS: 9056-9066 |
| MTX2 | Metaxin 2 | SEQ ID NOS: 9067-9071 |
| MUC1 | Mucin 1, cell surface associated | SEQ ID NOS: 9072-9097 |
| MUC13 | Mucin 13, cell surface associated | SEQ ID NOS: 9098-9099 |
| MUC20 | Mucin 20, cell surface associated | SEQ ID NOS: 9100-9104 |
| MUC3A | Mucin 3A, cell surface associated | SEQ ID NOS: 9105-9107 |
| MUC5AC | Mucin 5AC, oligomeric mucus/gel-forming | SEQ ID NO: 9108 |
| MUC5B | Mucin 5B, oligomeric mucus/gel-forming | SEQ ID NOS: 9109-9110 |
| MUC6 | Mucin 6, oligomeric mucus/gel-forming | SEQ ID NOS: 9111-9114 |
| MUC7 | Mucin 7, secreted | SEQ ID NOS: 9115-9118 |
| MUCL1 | Mucin-like 1 | SEQ ID NOS: 9119-9121 |
| MXRA5 | Matrix-remodelling associated 5 | SEQ ID NO: 9122 |
| MXRA7 | Matrix-remodelling associated 7 | SEQ ID NOS: 9123-9129 |
| MYDGF | Myeloid-derived growth factor | SEQ ID NOS: 9130-9132 |
| MYL1 | Myosin, light chain 1, alkali; skeletal, fast | SEQ ID NOS: 9133-9134 |
| MYOC | Myocilin, trabecular meshwork inducible | SEQ ID NOS: 9135-9136 |
| | glucocorticoid response | |
| MYRFL | Myelin regulatory factor-like | SEQ ID NOS: 9137-9141 |
| MZB1 | Marginal zone B and B1 cell-specific protein | SEQ ID NOS: 9142-9146 |
| N4BP2L2 | NEDD4 binding protein 2-like 2 | SEQ ID NOS: 9147-9152 |
| NAA38 | N(alpha)-acetyltransferase 38, NatC auxiliary subunit | SEQ ID NOS: 9153-9158 |
| NAAA | N-acylethanolamine acid amidase | SEQ ID NOS: 9159-9164 |
| NAGA | N-acetylgalactosaminidase, alpha- | SEQ ID NOS: 9165-9167 |
| NAGLU | N-acetylglucosaminidase, alpha | SEQ ID NOS: 9168-9172 |
| NAGS | N-acetylglutamate synthase | SEQ ID NOS: 9173-9174 |
| NAPSA | Napsin A aspartic peptidase | SEQ ID NOS: 9175-9177 |
| CARKD | Carbohydrate kinase domain containing | SEQ ID NOS: 9178-9179 |
| APOA1BP | Apolipoprotein A-I binding protein | SEQ ID NOS: 9180-9182 |
| NBL1 | Neuroblastoma 1, DAN family BMP antagonist | SEQ ID NOS: 9183-9196 |
| NCAM1 | Neural cell adhesion molecule 1 | SEQ ID NOS: 9197-9216 |
| NCAN | Neurocan | SEQ ID NOS: 9217-9218 |
| NCBP2-AS2 | NCBP2 antisense RNA 2 (head to head) | SEQ ID NO: 9219 |
| NCSTN | Nicastrin | SEQ ID NOS: 9220-9229 |
| NDNF | Neuron-derived neurotrophic factor | SEQ ID NOS: 9230-9232 |
| NDP | Norrie disease (pseudoglioma) | SEQ ID NOS: 9233-9235 |
| NDUFA10 | NADH dehydrogenase (ubiquinone) 1 alpha | SEQ ID NOS: 9236-9245 |
| | subcomplex, 10, 42 kDa | |
| NDUFB5 | NADH dehydrogenase (ubiquinone) 1 beta | SEQ ID NOS: 9246-9254 |
| | subcomplex, 5, 16 kDa | |
| NDUFS8 | NADH dehydrogenase (ubiquinone) Fe—S protein 8, | SEQ ID NOS: 9255-9264 |
| | 23 kDa (NADH-coenzyme Q reductase) | |
| NDUFV1 | NADH dehydrogenase (ubiquinone) flavoprotein 1, | SEQ ID NOS: 9265-9278 |
| | 51 kDa | |
| NECAB3 | N-terminal EF-hand calcium binding protein 3 | SEQ ID NOS: 9279-9288 |
| PVRL1 | Poliovirus receptor-related 1 (herpesvirus entry | SEQ ID NOS: 9289-9291 |
| | mediator C) | |
| NELL1 | Neural EGFL like 1 | SEQ ID NOS: 9292-9295 |
| NELL2 | Neural EGFL like 2 | SEQ ID NOS: 9296-9310 |
| NENF | Neudesin neurotrophic factor | SEQ ID NO: 9311 |
| NETO1 | Neuropilin (NRP) and tolloid (TLL)-like 1 | SEQ ID NOS: 9312-9316 |
| NFASC | Neurofascin | SEQ ID NOS: 9317-9331 |
| NFE2L1 | Nuclear factor, erythroid 2-like 1 | SEQ ID NOS: 9332-9350 |
| NFE2L3 | Nuclear factor, erythroid 2-like 3 | SEQ ID NOS: 9351-9352 |
| NGEF | Neuronal guanine nucleotide exchange factor | SEQ ID NOS: 9353-9358 |
| NGF | Nerve growth factor (beta polypeptide) | SEQ ID NO: 9359 |
| NGLY1 | N-glycanase 1 | SEQ ID NOS: 9360-9366 |
| NGRN | Neugrin, neurite outgrowth associated | SEQ ID NOS: 9367-9368 |
| NHLRC3 | NHL repeat containing 3 | SEQ ID NOS: 9369-9371 |
| NID1 | Nidogen 1 | SEQ ID NOS: 9372-9373 |
| NID2 | Nidogen 2 (osteonidogen) | SEQ ID NOS: 9374-9376 |
| NKG7 | Natural killer cell granule protein 7 | SEQ ID NOS: 9377-9381 |
| NLGN3 | Neuroligin 3 | SEQ ID NOS: 9382-9386 |
| NLGN4Y | Neuroligin 4, Y-linked | SEQ ID NOS: 9387-9393 |
| NLRP5 | NLR family, pyrin domain containing 5 | SEQ ID NOS: 9394-9396 |
| NMB | Neuromedin B | SEQ ID NOS: 9397-9398 |
| NME1 | NME/NM23 nucleoside diphosphate kinase 1 | SEQ ID NOS: 9399-9405 |
| NME1- | NME1-NME2 readthrough | SEQ ID NOS: 9406-9408 |
| NME2 | | |
| NME3 | NME/NM23 nucleoside diphosphate kinase 3 | SEQ ID NOS: 9409-9413 |
| NMS | Neuromedin S | SEQ ID NO: 9414 |
| NMU | Neuromedin U | SEQ ID NOS: 9415-9418 |
| NOA1 | Nitric oxide associated 1 | SEQ ID NO: 9419 |
| NODAL | Nodal growth differentiation factor | SEQ ID NOS: 9420-9421 |
| NOG | Noggin | SEQ ID NO: 9422 |
| NOMO3 | NODAL modulator 3 | SEQ ID NOS: 9423-9429 |
| NOS1AP | Nitric oxide synthase 1 (neuronal) adaptor protein | SEQ ID NOS: 9430-9434 |
| NOTCH3 | Notch 3 | SEQ ID NOS: 9435-9438 |
| NOTUM | Notum pectinacetylesterase homolog (Drosophila) | SEQ ID NOS: 9439-9441 |
| NOV | Nephroblastoma overexpressed | SEQ ID NO: 9442 |
| NPB | Neuropeptide B | SEQ ID NOS: 9443-9444 |
| NPC2 | Niemann-Pick disease, type C2 | SEQ ID NOS: 9445-9453 |
| NPFF | Neuropeptide FF-amide peptide precursor | SEQ ID NO: 9454 |
| NPFFR2 | Neuropeptide FF receptor 2 | SEQ ID NOS: 9455-9458 |
| NPHS1 | Nephrosis 1, congenital, Finnish type (nephrin) | SEQ ID NOS: 9459-9460 |
| NPNT | Nephronectin | SEQ ID NOS: 9461-9471 |
| NPPA | Natriuretic peptide A | SEQ ID NOS: 9472-9474 |
| NPPB | Natriuretic peptide B | SEQ ID NO: 9475 |
| NPPC | Natriuretic peptide C | SEQ ID NOS: 9476-9477 |
| NPS | Neuropeptide S | SEQ ID NO: 9478 |
| NPTX1 | Neuronal pentraxin I | SEQ ID NO: 9479 |
| NPTX2 | Neuronal pentraxin II | SEQ ID NO: 9480 |
| NPTXR | Neuronal pentraxin receptor | SEQ ID NOS: 9481-9482 |
| NPVF | Neuropeptide VF precursor | SEQ ID NO: 9483 |
| NPW | Neuropeptide W | SEQ ID NOS: 9484-9486 |
| NPY | Neuropeptide Y | SEQ ID NOS: 9487-9489 |
| NQO2 | NAD(P)H dehydrogenase, quinone 2 | SEQ ID NOS: 9490-9498 |
| NRCAM | Neuronal cell adhesion molecule | SEQ ID NOS: 9499-9511 |
| NRG1 | Neuregulin 1 | SEQ ID NOS: 9512-9529 |
| NRN1L | Neuritin 1-like | SEQ ID NOS: 9530-9532 |
| NRP1 | Neuropilin 1 | SEQ ID NOS: 9533-9546 |
| NRP2 | Neuropilin 2 | SEQ ID NOS: 9547-9553 |
| NRTN | Neurturin | SEQ ID NO: 9554 |
| NRXN1 | Neurexin 1 | SEQ ID NOS: 9555-9585 |
| NRXN2 | Neurexin 2 | SEQ ID NOS: 9586-9594 |
| NT5C3A | 5′-nucleotidase, cytosolic IIIA | SEQ ID NOS: 9595-9605 |
| NT5DC3 | 5′-nucleotidase domain containing 3 | SEQ ID NOS: 9606-9608 |
| NT5E | 5′-nucleotidase, ecto (CD73) | SEQ ID NOS: 9609-9613 |
| NTF3 | Neurotrophin 3 | SEQ ID NOS: 9614-9615 |
| NTF4 | Neurotrophin 4 | SEQ ID NOS: 9616-9617 |
| NTM | Neurotrimin | SEQ ID NOS: 9618-9627 |
| NTN1 | Netrin 1 | SEQ ID NOS: 9628-9629 |
| NTN3 | Netrin 3 | SEQ ID NO: 9630 |
| NTN4 | Netrin 4 | SEQ ID NOS: 9631-9635 |
| NTN5 | Netrin 5 | SEQ ID NOS: 9636-9637 |
| NTNG1 | Netrin G1 | SEQ ID NOS: 9638-9644 |
| NTNG2 | Netrin G2 | SEQ ID NOS: 9645-9646 |
| NTS | Neurotensin | SEQ ID NOS: 9647-9648 |
| NUBPL | Nucleotide binding protein-like | SEQ ID NOS: 9649-9655 |
| NUCB1 | Nucleobindin 1 | SEQ ID NOS: 9656-9662 |
| NUCB2 | Nucleobindin 2 | SEQ ID NOS: 9663-9678 |
| NUDT19 | Nudix (nucleoside diphosphate linked moiety X)-type | SEQ ID NO: 9679 |
| | motif 19 | |
| NUDT9 | Nudix (nucleoside diphosphate linked moiety X)-type | SEQ ID NOS: 9680-9684 |
| | motif 9 | |
| NUP155 | Nucleoporin 155 kDa | SEQ ID NOS: 9685-9688 |
| NUP214 | Nucleoporin 214 kDa | SEQ ID NOS: 9689-9700 |
| NUP85 | Nucleoporin 85 kDa | SEQ ID NOS: 9701-9715 |
| NXPE3 | Neurexophilin and PC-esterase domain family, | SEQ ID NOS: 9716-9721 |
| | member 3 | |
| NXPE4 | Neurexophilin and PC-esterase domain family, | SEQ ID NOS: 9722-9723 |
| | member 4 | |
| NXPH1 | Neurexophilin 1 | SEQ ID NOS: 9724-9727 |
| NXPH2 | Neurexophilin 2 | SEQ ID NO: 9728 |
| NXPH3 | Neurexophilin 3 | SEQ ID NOS: 9729-9730 |
| NXPH4 | Neurexophilin 4 | SEQ ID NOS: 9731-9732 |
| NYX | Nyctalopin | SEQ ID NOS: 9733-9734 |
| OAF | Out at first homolog | SEQ ID NOS: 9735-9736 |
| OBP2A | Odorant binding protein 2A | SEQ ID NOS: 9737-9743 |
| OBP2B | Odorant binding protein 2B | SEQ ID NOS: 9744-9747 |
| OC90 | Otoconin 90 | SEQ ID NO: 9748 |
| OCLN | Occludin | SEQ ID NOS: 9749-9751 |
| ODAM | Odontogenic, ameloblast asssociated | SEQ ID NOS: 9752-9755 |
| C4orf26 | Chromosome 4 open reading frame 26 | SEQ ID NOS: 9756-9759 |
| OGG1 | 8-oxoguanine DNA glycosylase | SEQ ID NOS: 9760-9773 |
| OGN | Osteoglycin | SEQ ID NOS: 9774-9776 |
| OIT3 | Oncoprotein induced transcript 3 | SEQ ID NOS: 9777-9778 |
| OLFM1 | Olfactomedin 1 | SEQ ID NOS: 9779-9789 |
| OLFM2 | Olfactomedin 2 | SEQ ID NOS: 9790-9793 |
| OLFM3 | Olfactomedin 3 | SEQ ID NOS: 9794-9796 |
| OLFM4 | Olfactomedin 4 | SEQ ID NO: 9797 |
| OLFML1 | Olfactomedin-like 1 | SEQ ID NOS: 9798-9801 |
| OLFML2A | Olfactomedin-like 2A | SEQ ID NOS: 9802-9804 |
| OLFML2B | Olfactomedin-like 2B | SEQ ID NOS: 9805-9809 |
| OLFML3 | Olfactomedin-like 3 | SEQ ID NOS: 9810-9812 |
| OMD | Osteomodulin | SEQ ID NO: 9813 |
| OMG | Oligodendrocyte myelin glycoprotein | SEQ ID NO: 9814 |
| OOSP2 | Oocyte secreted protein 2 | SEQ ID NOS: 9815-9816 |
| OPCML | Opioid binding protein/cell adhesion molecule-like | SEQ ID NOS: 9817-9821 |
| PROL1 | Proline rich, lacrimal 1 | SEQ ID NO: 9822 |
| OPTC | Opticin | SEQ ID NOS: 9823-9824 |
| ORAI1 | ORAI calcium release-activated calcium modulator 1 | SEQ ID NO: 9825 |
| ORM1 | Orosomucoid 1 | SEQ ID NO: 9826 |
| ORM2 | Orosomucoid 2 | SEQ ID NO: 9827 |
| ORMDL2 | ORMDL sphingolipid biosynthesis regulator 2 | SEQ ID NOS: 9828-9831 |
| OS9 | Osteosarcoma amplified 9, endoplasmic reticulum | SEQ ID NOS: 9832-9846 |
| | lectin | |
| OSCAR | Osteoclast associated, immunoglobulin-like receptor | SEQ ID NOS: 9847-9857 |
| OSM | Oncostatin M | SEQ ID NOS: 9858-9860 |
| OSMR | Oncostatin M receptor | SEQ ID NOS: 9861-9865 |
| OSTN | Osteocrin | SEQ ID NOS: 9866-9867 |
| OTOA | Otoancorin | SEQ ID NOS: 9868-9873 |
| OTOG | Otogelin | SEQ ID NOS: 9874-9876 |
| OTOGL | Otogelin-like | SEQ ID NOS: 9877-9883 |
| OTOL1 | Otolin 1 | SEQ ID NO: 9884 |
| OTOR | Otoraplin | SEQ ID NO: 9885 |
| OTOS | Otospiralin | SEQ ID NOS: 9886-9887 |
| OVCH1 | Ovochymase 1 | SEQ ID NOS: 9888-9890 |
| OVCH2 | Ovochymase 2 (gene/pseudogene) | SEQ ID NOS: 9891-9892 |
| OVGP1 | Oviductal glycoprotein 1, 120 kDa | SEQ ID NO: 9893 |
| OXCT1 | 3-oxoacid CoA transferase 1 | SEQ ID NOS: 9894-9897 |
| OXCT2 | 3-oxoacid CoA transferase 2 | SEQ ID NO: 9898 |
| OXNAD1 | Oxidoreductase NAD-binding domain containing 1 | SEQ ID NOS: 9899-9905 |
| OXT | Oxytocin/neurophysin I prepropeptide | SEQ ID NO: 9906 |
| P3H1 | Prolyl 3-hydroxylase 1 | SEQ ID NOS: 9907-9911 |
| P3H2 | Prolyl 3-hydroxylase 2 | SEQ ID NOS: 9912-9915 |
| P3H3 | Prolyl 3-hydroxylase 3 | SEQ ID NO: 9916 |
| P3H4 | Prolyl 3-hydroxylase family member 4 (non- | SEQ ID NOS: 9917-9921 |
| | enzymatic) | |
| P4HA1 | Prolyl 4-hydroxylase, alpha polypeptide I | SEQ ID NOS: 9922-9926 |
| P4HA2 | Prolyl 4-hydroxylase, alpha polypeptide II | SEQ ID NOS: 9927-9941 |
| P4HA3 | Prolyl 4-hydroxylase, alpha polypeptide III | SEQ ID NOS: 9942-9946 |
| P4HB | Prolyl 4-hydroxylase, beta polypeptide | SEQ ID NOS: 9947-9958 |
| PAEP | Progestagen-associated endometrial protein | SEQ ID NOS: 9959-9967 |
| PAM | Peptidylglycine alpha-amidating monooxygenase | SEQ ID NOS: 9968-9981 |
| PAMR1 | Peptidase domain containing associated with muscle | SEQ ID NOS: 9982-9988 |
| | regeneration 1 | |
| PAPLN | Papilin, proteoglycan-like sulfated glycoprotein | SEQ ID NOS: 9989-9996 |
| PAPPA | Pregnancy-associated plasma protein A, | SEQ ID NO: 9997 |
| | pappalysin 1 | |
| PAPPA2 | Pappalysin 2 | SEQ ID NOS: 9998-9999 |
| PARP15 | Poly (ADP-ribose) polymerase family, member 15 | SEQ ID NOS: 10000-10003 |
| PARVB | Parvin, beta | SEQ ID NOS: 10004-10008 |
| PATE1 | Prostate and testis expressed 1 | SEQ ID NOS: 10009-10010 |
| PATE2 | Prostate and testis expressed 2 | SEQ ID NOS: 10011-10012 |
| PATE3 | Prostate and testis expressed 3 | SEQ ID NO: 10013 |
| PATE4 | Prostate and testis expressed 4 | SEQ ID NOS: 10014-10015 |
| PATL2 | Protein associated with topoisomerase II homolog 2 | SEQ ID NOS: 10016-10021 |
| | (yeast) | |
| PAX2 | Paired box 2 | SEQ ID NOS: 10022-10027 |
| PAX4 | Paired box 4 | SEQ ID NOS: 10028-10034 |
| PCCB | Propionyl CoA carboxylase, beta polypeptide | SEQ ID NOS: 10035-10049 |
| PCDH1 | Protocadherin 1 | SEQ ID NOS: 10050-10055 |
| PCDH12 | Protocadherin 12 | SEQ ID NOS: 10056-10057 |
| PCDH15 | Protocadherin-related 15 | SEQ ID NOS: 10058-10091 |
| PCDHA1 | Protocadherin alpha 1 | SEQ ID NOS: 10092-10094 |
| PCDHA10 | Protocadherin alpha 10 | SEQ ID NOS: 10095-10097 |
| PCDHA11 | Protocadherin alpha 11 | SEQ ID NOS: 10098-10100 |
| PCDHA6 | Protocadherin alpha 6 | SEQ ID NOS: 10101-10103 |
| PCDHB12 | Protocadherin beta 12 | SEQ ID NOS: 10104-10106 |
| PCDHGA11 | Protocadherin gamma subfamily A, 11 | SEQ ID NOS: 10107-10109 |
| PCF11 | PCF11 cleavage and polyadenylation factor subunit | SEQ ID NOS: 10110-10114 |
| PCOLCE | Procollagen C-endopeptidase enhancer | SEQ ID NO: 10115 |
| PCOLCE2 | Procollagen C-endopeptidase enhancer 2 | SEQ ID NOS: 10116-10119 |
| PCSK1 | Proprotein convertase subtilisin/kexin type 1 | SEQ ID NOS: 10120-10122 |
| PCSK1N | Proprotein convertase subtilisin/kexin type 1 | SEQ ID NO: 10123 |
| | inhibitor | |
| PCSK2 | Proprotein convertase subtilisin/kexin type 2 | SEQ ID NOS: 10124-10126 |
| PCSK4 | Proprotein convertase subtilisin/kexin type 4 | SEQ ID NOS: 10127-10129 |
| PCSK5 | Proprotein convertase subtilisin/kexin type 5 | SEQ ID NOS: 10130-10134 |
| PCSK9 | Proprotein convertase subtilisin/kexin type 9 | SEQ ID NO: 10135 |
| PCYOX1 | Prenylcysteine oxidase 1 | SEQ ID NOS: 10136-10140 |
| PCYOX1L | Prenylcysteine oxidase 1 like | SEQ ID NOS: 10141-10145 |
| PDE11A | Phosphodiesterase 11A | SEQ ID NOS: 10146-10151 |
| PDE2A | Phosphodiesterase 2A, cGMP-stimulated | SEQ ID NOS: 10152-10173 |
| PDE7A | Phosphodiesterase 7A | SEQ ID NOS: 10174-10177 |
| PDF | Peptide deformylase (mitochondrial) | SEQ ID NO: 10178 |
| PDGFA | Platelet-derived growth factor alpha polypeptide | SEQ ID NOS: 10179-10182 |
| PDGFB | Platelet-derived growth factor beta polypeptide | SEQ ID NOS: 10183-10186 |
| PDGFC | Platelet derived growth factor C | SEQ ID NOS: 10187-10190 |
| PDGFD | Platelet derived growth factor D | SEQ ID NOS: 10191-10193 |
| PDGFRA | Platelet-derived growth factor receptor, alpha | SEQ ID NOS: 10194-10200 |
| | polypeptide | |
| PDGFRB | Platelet-derived growth factor receptor, beta | SEQ ID NOS: 10201-10204 |
| | polypeptide | |
| PDGFRL | Platelet-derived growth factor receptor-like | SEQ ID NOS: 10205-10206 |
| PDHA1 | Pyruvate dehydrogenase (lipoamide) alpha 1 | SEQ ID NOS: 10207-10215 |
| PDIA2 | Protein disulfide isomerase family A, member 2 | SEQ ID NOS: 10216-10219 |
| PDIA3 | Protein disulfide isomerase family A, member 3 | SEQ ID NOS: 10220-10223 |
| PDIA4 | Protein disulfide isomerase family A, member 4 | SEQ ID NOS: 10224-10225 |
| PDIA5 | Protein disulfide isomerase family A, member 5 | SEQ ID NOS: 10226-10229 |
| PDIA6 | Protein disulfide isomerase family A, member 6 | SEQ ID NOS: 10230-10236 |
| PDILT | Protein disulfide isomerase-like, testis expressed | SEQ ID NOS: 10237-10238 |
| PDYN | Prodynorphin | SEQ ID NOS: 10239-10241 |
| PDZD8 | PDZ domain containing 8 | SEQ ID NO: 10242 |
| PDZRN4 | PDZ domain containing ring finger 4 | SEQ ID NOS: 10243-10245 |
| PEAR1 | Platelet endothelial aggregation receptor 1 | SEQ ID NOS: 10246-10249 |
| PEBP4 | Phosphatidylethanolamine-binding protein 4 | SEQ ID NOS: 10250-10251 |
| PECAM1 | Platelet/endothelial cell adhesion molecule 1 | SEQ ID NOS: 10252-10255 |
| PENK | Proenkephalin | SEQ ID NOS: 10256-10261 |
| PET117 | PET117 homolog | SEQ ID NO: 10262 |
| PF4 | Platelet factor 4 | SEQ ID NO: 10263 |
| PF4V1 | Platelet factor 4 variant 1 | SEQ ID NO: 10264 |
| PFKP | Phosphofructokinase, platelet | SEQ ID NOS: 10265-10273 |
| PFN1 | Profilin 1 | SEQ ID NOS: 10274-10276 |
| PGA3 | Pepsinogen 3, group I (pepsinogen A) | SEQ ID NOS: 10277-10280 |
| PGA4 | Pepsinogen 4, group I (pepsinogen A) | SEQ ID NOS: 10281-10283 |
| PGA5 | Pepsinogen 5, group I (pepsinogen A) | SEQ ID NOS: 10284-10286 |
| PGAM5 | PGAM family member 5, serine/threonine protein | SEQ ID NOS: 10287-10290 |
| | phosphatase, mitochondrial | |
| PGAP3 | Post-GPI attachment to proteins 3 | SEQ ID NOS: 10291-10298 |
| PGC | Progastricsin (pepsinogen C) | SEQ ID NOS: 10299-10302 |
| PGF | Placental growth factor | SEQ ID NOS: 10303-10306 |
| PGLYRP1 | Peptidoglycan recognition protein 1 | SEQ ID NO: 10307 |
| PGLYRP2 | Peptidoglycan recognition protein 2 | SEQ ID NOS: 10308-10311 |
| PGLYRP3 | Peptidoglycan recognition protein 3 | SEQ ID NO: 10312 |
| PGLYRP4 | Peptidoglycan recognition protein 4 | SEQ ID NOS: 10313-10314 |
| PHACTR1 | Phosphatase and actin regulator 1 | SEQ ID NOS: 10315-10321 |
| PHB | Prohibitin | SEQ ID NOS: 10322-10330 |
| PI15 | Peptidase inhibitor 15 | SEQ ID NOS: 10331-10332 |
| PI3 | Peptidase inhibitor 3, skin-derived | SEQ ID NO: 10333 |
| PIANP | PILR alpha associated neural protein | SEQ ID NOS: 10334-10339 |
| PIGK | Phosphatidylinositol glycan anchor biosynthesis, | SEQ ID NOS: 10340-10343 |
| | class K | |
| PIGL | Phosphatidylinositol glycan anchor biosynthesis, | SEQ ID NOS: 10344-10351 |
| | class L | |
| PIGT | Phosphatidylinositol glycan anchor biosynthesis, | SEQ ID NOS: 10352-10406 |
| | class T | |
| PIGZ | Phosphatidylinositol glycan anchor biosynthesis, | SEQ ID NOS: 10407-10409 |
| | class Z | |
| PIK3AP1 | Phosphoinositide-3-kinase adaptor protein 1 | SEQ ID NOS: 10410-10412 |
| PIK3IP1 | Phosphoinositide-3-kinase interacting protein 1 | SEQ ID NOS: 10413-10416 |
| PILRA | Paired immunoglobin-like type 2 receptor alpha | SEQ ID NOS: 10417-10421 |
| PILRB | Paired immunoglobin-like type 2 receptor beta | SEQ ID NOS: 10422-10433 |
| PINLYP | Phospholipase A2 inhibitor and LY6/PLAUR domain | SEQ ID NOS: 10434-10438 |
| | containing | |
| PIP | Prolactin-induced protein | SEQ ID NO: 10439 |
| PIWIL4 | Piwi-like RNA-mediated gene silencing 4 | SEQ ID NOS: 10440-10444 |
| PKDCC | Protein kinase domain containing, cytoplasmic | SEQ ID NOS: 10445-10446 |
| PKHD1 | Polycystic kidney and hepatic disease 1 (autosomal | SEQ ID NOS: 10447-10448 |
| | recessive) | |
| PLA1A | Phospholipase A1 member A | SEQ ID NOS: 10449-10453 |
| PLA2G10 | Phospholipase A2, group X | SEQ ID NOS: 10454-10455 |
| PLA2G12A | Phospholipase A2, group XIIA | SEQ ID NOS: 10456-10458 |
| PLA2G12B | Phospholipase A2, group XIIB | SEQ ID NO: 10459 |
| PLA2G15 | Phospholipase A2, group XV | SEQ ID NOS: 10460-10467 |
| PLA2G1B | Phospholipase A2, group IB (pancreas) | SEQ ID NOS: 10468-10470 |
| PLA2G2A | Phospholipase A2, group IIA (platelets, synovial | SEQ ID NOS: 10471-10472 |
| | fluid) | |
| PLA2G2C | Phospholipase A2, group IIC | SEQ ID NOS: 10473-10474 |
| PLA2G2D | Phospholipase A2, group IID | SEQ ID NOS: 10475-10476 |
| PLA2G2E | Phospholipase A2, group IIE | SEQ ID NO: 10477 |
| PLA2G3 | Phospholipase A2, group III | SEQ ID NO: 10478 |
| PLA2G5 | Phospholipase A2, group V | SEQ ID NO: 10479 |
| PLA2G7 | Phospholipase A2, group VII (platelet-activating | SEQ ID NOS: 10480-10481 |
| | factor acetylhydrolase, plasma) | |
| PLA2R1 | Phospholipase A2 receptor 1, 180 kDa | SEQ ID NOS: 10482-10483 |
| PLAC1 | Placenta-specific 1 | SEQ ID NO: 10484 |
| PLAC9 | Placenta-specific 9 | SEQ ID NOS: 10485-10487 |
| PLAT | Plasminogen activator, tissue | SEQ ID NOS: 10488-10496 |
| PLAU | Plasminogen activator, urokinase | SEQ ID NOS: 10497-10499 |
| PLAUR | Plasminogen activator, urokinase receptor | SEQ ID NOS: 10500-10511 |
| PLBD1 | Phospholipase B domain containing 1 | SEQ ID NOS: 10512-10514 |
| PLBD2 | Phospholipase B domain containing 2 | SEQ ID NOS: 10515-10517 |
| PLG | Plasminogen | SEQ ID NOS: 10518-10520 |
| PLGLB1 | Plasminogen-like B1 | SEQ ID NOS: 10521-10524 |
| PLGLB2 | Plasminogen-like B2 | SEQ ID NOS: 10525-10526 |
| PLOD1 | Procollagen-lysine, 2-oxoglutarate 5-dioxygenase 1 | SEQ ID NOS: 10527-10529 |
| PLOD2 | Procollagen-lysine, 2-oxoglutarate 5-dioxygenase 2 | SEQ ID NOS: 10530-10535 |
| PLOD3 | Procollagen-lysine, 2-oxoglutarate 5-dioxygenase 3 | SEQ ID NOS: 10536-10542 |
| PLTP | Phospholipid transfer protein | SEQ ID NOS: 10543-10547 |
| PLXNA4 | Plexin A4 | SEQ ID NOS: 10548-10551 |
| PLXNB2 | Plexin B2 | SEQ ID NOS: 10552-10560 |
| PM20D1 | Peptidase M20 domain containing 1 | SEQ ID NO: 10561 |
| PMCH | Pro-melanin-concentrating hormone | SEQ ID NO: 10562 |
| PMEL | Premelanosome protein | SEQ ID NOS: 10563-10574 |
| PMEPA1 | Prostate transmembrane protein, androgen | SEQ ID NOS: 10575-10581 |
| | induced 1 | |
| PNLIP | Pancreatic lipase | SEQ ID NO: 10582 |
| PNLIPRP1 | Pancreatic lipase-related protein 1 | SEQ ID NOS: 10583-10591 |
| PNLIPRP3 | Pancreatic lipase-related protein 3 | SEQ ID NO: 10592 |
| PNOC | Prepronociceptin | SEQ ID NOS: 10593-10595 |
| PNP | Purine nucleoside phosphorylase | SEQ ID NOS: 10596-10599 |
| PNPLA4 | Patatin-like phospholipase domain containing 4 | SEQ ID NOS: 10600-10603 |
| PODNL1 | Podocan-like 1 | SEQ ID NOS: 10604-10615 |
| POFUT1 | Protein O-fucosyltransferase 1 | SEQ ID NOS: 10616-10617 |
| POFUT2 | Protein O-fucosyltransferase 2 | SEQ ID NOS: 10618-10623 |
| POGLUT1 | Protein O-glucosyltransferase 1 | SEQ ID NOS: 10624-10628 |
| POLL | Polymerase (DNA directed), lambda | SEQ ID NOS: 10629-10641 |
| POMC | Proopiomelanocortin | SEQ ID NOS: 10642-10646 |
| POMGNT2 | Protein O-linked mannose N- | SEQ ID NOS: 10647-10648 |
| | acetylglucosaminyltransferase 2 (beta 1,4-) | |
| PON1 | Paraoxonase 1 | SEQ ID NOS: 10649-10650 |
| PON2 | Paraoxonase 2 | SEQ ID NOS: 10651-10663 |
| PON3 | Paraoxonase 3 | SEQ ID NOS: 10664-10669 |
| POSTN | Periostin, osteoblast specific factor | SEQ ID NOS: 10670-10675 |
| PPBP | Pro-platelet basic protein (chemokine (C-X-C motif) | SEQ ID NO: 10676 |
| | ligand 7) | |
| PPIB | Peptidylprolyl isomerase B (cyclophilin B) | SEQ ID NO: 10677 |
| PPIC | Peptidylprolyl isomerase C (cyclophilin C) | SEQ ID NO: 10678 |
| PPOX | Protoporphyrinogen oxidase | SEQ ID NOS: 10679-10689 |
| PPP1CA | Protein phosphatase 1, catalytic subunit, alpha | SEQ ID NOS: 10690-10695 |
| | isozyme | |
| PPT1 | Palmitoyl-protein thioesterase 1 | SEQ ID NOS: 10696-10712 |
| PPT2 | Palmitoyl-protein thioesterase 2 | SEQ ID NOS: 10713-10720 |
| PPY | Pancreatic polypeptide | SEQ ID NOS: 10721-10725 |
| PRAC2 | Prostate cancer susceptibility candidate 2 | SEQ ID NOS: 10726-10727 |
| PRADC1 | Protease-associated domain containing 1 | SEQ ID NO: 10728 |
| PRAP1 | Proline-rich acidic protein 1 | SEQ ID NOS: 10729-10730 |
| PRB1 | Proline-rich protein BstNI subfamily 1 | SEQ ID NOS: 10731-10734 |
| PRB2 | Proline-rich protein BstNI subfamily 2 | SEQ ID NOS: 10735-10736 |
| PRB3 | Proline-rich protein BstNI subfamily 3 | SEQ ID NOS: 10737-10738 |
| PRB4 | Proline-rich protein BstNI subfamily 4 | SEQ ID NOS: 10739-10742 |
| PRCD | Progressive rod-cone degeneration | SEQ ID NOS: 10743-10744 |
| PRCP | Prolylcarboxypeptidase (angiotensinase C) | SEQ ID NOS: 10745-10756 |
| PRDM12 | PR domain containing 12 | SEQ ID NO: 10757 |
| PRDX4 | Peroxiredoxin 4 | SEQ ID NOS: 10758-10761 |
| PRELP | Proline/arginine-rich end leucine-rich repeat protein | SEQ ID NO: 10762 |
| PRF1 | Perforin 1 (pore forming protein) | SEQ ID NOS: 10763-10765 |
| PRG2 | Proteoglycan 2, bone marrow (natural killer cell | SEQ ID NOS: 10766-10768 |
| | activator, eosinophil granule major basic protein) | |
| PRG3 | Proteoglycan 3 | SEQ ID NO: 10769 |
| PRG4 | Proteoglycan 4 | SEQ ID NOS: 10770-10775 |
| PRH1 | Proline-rich protein Haelll subfamily 1 | SEQ ID NOS: 10776-10778 |
| PRH2 | Proline-rich protein Haelll subfamily 2 | SEQ ID NOS: 10779-10780 |
| PRKAG1 | Protein kinase, AMP-activated, gamma 1 non- | SEQ ID NOS: 10781-10795 |
| | catalytic subunit | |
| PRKCSH | Protein kinase C substrate 80K-H | SEQ ID NOS: 10796-10805 |
| PRKD1 | Protein kinase D1 | SEQ ID NOS: 10806-10811 |
| PRL | Prolactin | SEQ ID NOS: 10812-10814 |
| PRLH | Prolactin releasing hormone | SEQ ID NO: 10815 |
| PRLR | Prolactin receptor | SEQ ID NOS: 10816-10834 |
| PRNP | Prion protein | SEQ ID NOS: 10835-10838 |
| PRNT | Prion protein (testis specific) | SEQ ID NO: 10839 |
| PROC | Protein C (inactivator of coagulation factors Va and | SEQ ID NOS: 10840-10847 |
| | VIIIa) | |
| PROK1 | Prokineticin 1 | SEQ ID NO: 10848 |
| PROK2 | Prokineticin 2 | SEQ ID NOS: 10849-10850 |
| PROM1 | Prominin 1 | SEQ ID NOS: 10851-10862 |
| PROS1 | Protein S (alpha) | SEQ ID NOS: 10863-10866 |
| PROZ | Protein Z, vitamin K-dependent plasma glycoprotein | SEQ ID NOS: 10867-10868 |
| PRR27 | Proline rich 27 | SEQ ID NOS: 10869-10872 |
| PRR4 | Proline rich 4 (lacrimal) | SEQ ID NOS: 10873-10875 |
| PRRG2 | Proline rich Gla (G-carboxyglutamic acid) 2 | SEQ ID NOS: 10876-10878 |
| PRRT3 | Proline-rich transmembrane protein 3 | SEQ ID NOS: 10879-10881 |
| PRRT4 | Proline-rich transmembrane protein 4 | SEQ ID NOS: 10882-10888 |
| PRSS1 | Protease, serine, 1 (trypsin 1) | SEQ ID NOS: 10889-10892 |
| PRSS12 | Protease, serine, 12 (neurotrypsin, motopsin) | SEQ ID NO: 10893 |
| PRSS16 | Protease, serine, 16 (thymus) | SEQ ID NOS: 10894-10901 |
| PRSS2 | Protease, serine, 2 (trypsin 2) | SEQ ID NOS: 10902-10905 |
| PRSS21 | Protease, serine, 21 (testisin) | SEQ ID NOS: 10906-10911 |
| PRSS22 | Protease, serine, 22 | SEQ ID NOS: 10912-10914 |
| PRSS23 | Protease, serine, 23 | SEQ ID NOS: 10915-10918 |
| PRSS27 | Protease, serine 27 | SEQ ID NOS: 10919-10921 |
| PRSS3 | Protease, serine, 3 | SEQ ID NOS: 10922-10926 |
| PRSS33 | Protease, serine, 33 | SEQ ID NOS: 10927-10930 |
| PRSS35 | Protease, serine, 35 | SEQ ID NO: 10931 |
| PRSS36 | Protease, serine, 36 | SEQ ID NOS: 10932-10935 |
| PRSS37 | Protease, serine, 37 | SEQ ID NOS: 10936-10939 |
| PRSS38 | Protease, serine, 38 | SEQ ID NO: 10940 |
| PRSS42 | Protease, serine, 42 | SEQ ID NOS: 10941-10942 |
| PRSS48 | Protease, serine, 48 | SEQ ID NOS: 10943-10944 |
| PRSS50 | Protease, serine, 50 | SEQ ID NO: 10945 |
| PRSS53 | Protease, serine, 53 | SEQ ID NO: 10946 |
| PRSS54 | Protease, serine, 54 | SEQ ID NOS: 10947-10951 |
| PRSS55 | Protease, serine, 55 | SEQ ID NOS: 10952-10954 |
| PRSS56 | Protease, serine, 56 | SEQ ID NOS: 10955-10956 |
| PRSS57 | Protease, serine, 57 | SEQ ID NOS: 10957-10958 |
| PRSS58 | Protease, serine, 58 | SEQ ID NOS: 10959-10960 |
| PRSS8 | Protease, serine, 8 | SEQ ID NOS: 10961-10964 |
| PRTG | Protogenin | SEQ ID NOS: 10965-10968 |
| PRTN3 | Proteinase 3 | SEQ ID NOS: 10969-10970 |
| PSAP | Prosaposin | SEQ ID NOS: 10971-10974 |
| PSAPL1 | Prosaposin-like 1 (gene/pseudogene) | SEQ ID NO: 10975 |
| PSG1 | Pregnancy specific beta-1-glycoprotein 1 | SEQ ID NOS: 10976-10983 |
| PSG11 | Pregnancy specific beta-1-glycoprotein 11 | SEQ ID NOS: 10984-10988 |
| PSG2 | Pregnancy specific beta-1-glycoprotein 2 | SEQ ID NOS: 10989-10990 |
| PSG3 | Pregnancy specific beta-1-glycoprotein 3 | SEQ ID NOS: 10991-10994 |
| PSG4 | Pregnancy specific beta-1-glycoprotein 4 | SEQ ID NOS: 10995-11006 |
| PSG5 | Pregnancy specific beta-1-glycoprotein 5 | SEQ ID NOS: 11007-11012 |
| PSG6 | Pregnancy specific beta-1-glycoprotein 6 | SEQ ID NOS: 11013-11018 |
| PSG7 | Pregnancy specific beta-1-glycoprotein 7 | SEQ ID NOS: 11019-11021 |
| | (gene/pseudogene) | |
| PSG8 | Pregnancy specific beta-1-glycoprotein 8 | SEQ ID NOS: 11022-11026 |
| PSG9 | Pregnancy specific beta-1-glycoprotein 9 | SEQ ID NOS: 11027-11034 |
| PSMD1 | Proteasome 26S subunit, non-ATPase 1 | SEQ ID NOS: 11035-11042 |
| PSORS1C2 | Psoriasis susceptibility 1 candidate 2 | SEQ ID NO: 11043 |
| PSPN | Persephin | SEQ ID NOS: 11044-11045 |
| PTGDS | Prostaglandin D2 synthase 21 kDa (brain) | SEQ ID NOS: 11046-11050 |
| PTGIR | Prostaglandin I2 (prostacyclin) receptor (IP) | SEQ ID NOS: 11051-11055 |
| PTGS1 | Prostaglandin-endoperoxide synthase 1 | SEQ ID NOS: 11056-11064 |
| | (prostaglandin G/H synthase and cyclooxygenase) | |
| PTGS2 | Prostaglandin-endoperoxide synthase 2 | SEQ ID NOS: 11065-11066 |
| | (prostaglandin G/H synthase and cyclooxygenase) | |
| PTH | Parathyroid hormone | SEQ ID NOS: 11067-11068 |
| PTH2 | Parathyroid hormone 2 | SEQ ID NO: 11069 |
| PTHLH | Parathyroid hormone-like hormone | SEQ ID NOS: 11070-11078 |
| PTK7 | Protein tyrosine kinase 7 (inactive) | SEQ ID NOS: 11079-11094 |
| PTN | Pleiotrophin | SEQ ID NOS: 11095-11096 |
| PTPRA | Protein tyrosine phosphatase, receptor type, A | SEQ ID NOS: 11097-11104 |
| PTPRB | Protein tyrosine phosphatase, receptor type, B | SEQ ID NOS: 11105-11112 |
| PTPRC | Protein tyrosine phosphatase, receptor type, C | SEQ ID NOS: 11113-11123 |
| PTPRCAP | Protein tyrosine phosphatase, receptor type, C- | SEQ ID NO: 11124 |
| | associated protein | |
| PTPRD | Protein tyrosine phosphatase, receptor type, D | SEQ ID NOS: 11125-11136 |
| PTPRF | Protein tyrosine phosphatase, receptor type, F | SEQ ID NOS: 11137-11144 |
| PTPRJ | Protein tyrosine phosphatase, receptor type, J | SEQ ID NOS: 11145-11150 |
| PTPRO | Protein tyrosine phosphatase, receptor type, O | SEQ ID NOS: 11151-11159 |
| PTPRS | Protein tyrosine phosphatase, receptor type, S | SEQ ID NOS: 11160-11167 |
| PTTG1IP | Pituitary tumor-transforming 1 interacting protein | SEQ ID NOS: 11168-11171 |
| PTX3 | Pentraxin 3, long | SEQ ID NO: 11172 |
| PTX4 | Pentraxin 4, long | SEQ ID NOS: 11173-11175 |
| PVR | Poliovirus receptor | SEQ ID NOS: 11176-11181 |
| PXDN | Peroxidasin | SEQ ID NOS: 11182-11186 |
| PXDNL | Peroxidasin-like | SEQ ID NOS: 11187-11189 |
| PXYLP1 | 2-phosphoxylose phosphatase 1 | SEQ ID NOS: 11190-11202 |
| PYY | Peptide YY | SEQ ID NOS: 11203-11204 |
| PZP | Pregnancy-zone protein | SEQ ID NOS: 11205-11206 |
| QPCT | Glutaminyl-peptide cyclotransferase | SEQ ID NOS: 11207-11209 |
| QPRT | Quinolinate phosphoribosyltransferase | SEQ ID NOS: 11210-11211 |
| QRFP | Pyroglutamylated RFamide peptide | SEQ ID NOS: 11212-11213 |
| QSOX1 | Quiescin Q6 sulfhydryl oxidase 1 | SEQ ID NOS: 11214-11217 |
| R3HDML | R3H domain containing-like | SEQ ID NO: 11218 |
| RAB26 | RAB26, member RAS oncogene family | SEQ ID NOS: 11219-11222 |
| RAB36 | RAB36, member RAS oncogene family | SEQ ID NOS: 11223-11225 |
| RAB9B | RAB9B, member RAS oncogene family | SEQ ID NO: 11226 |
| RAET1E | Retinoic acid early transcript 1E | SEQ ID NOS: 11227-11232 |
| RAET1G | Retinoic acid early transcript 1G | SEQ ID NOS: 11233-11235 |
| RAMP2 | Receptor (G protein-coupled) activity modifying | SEQ ID NOS: 11236-11240 |
| | protein 2 | |
| RAPGEF5 | Rap guanine nucleotide exchange factor (GEF) 5 | SEQ ID NOS: 11241-11247 |
| RARRES1 | Retinoic acid receptor responder (tazarotene | SEQ ID NOS: 11248-11249 |
| | induced) 1 | |
| RARRES2 | Retinoic acid receptor responder (tazarotene | SEQ ID NOS: 11250-11253 |
| | induced) 2 | |
| RASA2 | RAS p21 protein activator 2 | SEQ ID NOS: 11254-11256 |
| RBM3 | RNA binding motif (RNP1, RRM) protein 3 | SEQ ID NOS: 11257-11259 |
| RBP3 | Retinol binding protein 3, interstitial | SEQ ID NO: 11260 |
| RBP4 | Retinol binding protein 4, plasma | SEQ ID NOS: 11261-11264 |
| RCN1 | Reticulocalbin 1, EF-hand calcium binding domain | SEQ ID NOS: 11265-11268 |
| RCN2 | Reticulocalbin 2, EF-hand calcium binding domain | SEQ ID NOS: 11269-11272 |
| RCN3 | Reticulocalbin 3, EF-hand calcium binding domain | SEQ ID NOS: 11273-11276 |
| RCOR1 | REST corepressor 1 | SEQ ID NOS: 11277-11278 |
| RDH11 | Retinol dehydrogenase 11 (all-trans/9-cis/11-cis) | SEQ ID NOS: 11279-11286 |
| RDH12 | Retinol dehydrogenase 12 (all-trans/9-cis/11-cis) | SEQ ID NOS: 11287-11288 |
| RDH13 | Retinol dehydrogenase 13 (all-trans/9-cis) | SEQ ID NOS: 11289-11297 |
| RDH5 | Retinol dehydrogenase 5 (11-cis/9-cis) | SEQ ID NOS: 11298-11302 |
| RDH8 | Retinol dehydrogenase 8 (all-trans) | SEQ ID NOS: 11303-11304 |
| REG1A | Regenerating islet-derived 1 alpha | SEQ ID NO: 11305 |
| REG1B | Regenerating islet-derived 1 beta | SEQ ID NOS: 11306-11307 |
| REG3A | Regenerating islet-derived 3 alpha | SEQ ID NOS: 11308-11310 |
| REG3G | Regenerating islet-derived 3 gamma | SEQ ID NOS: 11311-11313 |
| REG4 | Regenerating islet-derived family, member 4 | SEQ ID NOS: 11314-11317 |
| RELN | Reelin | SEQ ID NOS: 11318-11321 |
| RELT | RELT tumor necrosis factor receptor | SEQ ID NOS: 11322-11325 |
| REN | Renin | SEQ ID NOS: 11326-11327 |
| REPIN1 | Replication initiator 1 | SEQ ID NOS: 11328-11341 |
| REPS2 | RALBP1 associated Eps domain containing 2 | SEQ ID NOS: 11342-11343 |
| RET | Ret proto-oncogene | SEQ ID NOS: 11344-11349 |
| RETN | Resistin | SEQ ID NOS: 11350-11352 |
| RETNLB | Resistin like beta | SEQ ID NO: 11353 |
| RETSAT | Retinol saturase (all-trans-retinol 13,14-reductase) | SEQ ID NOS: 11354-11358 |
| RFNG | RFNG O-fucosylpeptide 3-beta-N- | SEQ ID NOS: 11359-11361 |
| | acetylglucosaminyltransferase | |
| RGCC | Regulator of cell cycle | SEQ ID NO: 11362 |
| RGL4 | Ral guanine nucleotide dissociation stimulator-like 4 | SEQ ID NOS: 11363-11369 |
| RGMA | Repulsive guidance molecule family member a | SEQ ID NOS: 11370-11379 |
| RGMB | Repulsive guidance molecule family member b | SEQ ID NOS: 11380-11381 |
| RHOQ | Ras homolog family member Q | SEQ ID NOS: 11382-11386 |
| RIC3 | RIC3 acetylcholine receptor chaperone | SEQ ID NOS: 11387-11394 |
| HRSP12 | Heat-responsive protein 12 | SEQ ID NOS: 11395-11398 |
| RIMS1 | Regulating synaptic membrane exocytosis 1 | SEQ ID NOS: 11399-11414 |
| RIPPLY1 | Ripply transcriptional repressor 1 | SEQ ID NOS: 11415-11416 |
| RLN1 | Relaxin 1 | SEQ ID NO: 11417 |
| RLN2 | Relaxin 2 | SEQ ID NOS: 11418-11419 |
| RLN3 | Relaxin 3 | SEQ ID NOS: 11420-11421 |
| RMDN1 | Regulator of microtubule dynamics 1 | SEQ ID NOS: 11422-11435 |
| RNASE1 | Ribonuclease, RNase A family, 1 (pancreatic) | SEQ ID NOS: 11436-11440 |
| RNASE10 | Ribonuclease, RNase A family, 10 (non-active) | SEQ ID NOS: 11441-11442 |
| RNASE11 | Ribonuclease, RNase A family, 11 (non-active) | SEQ ID NOS: 11443-11453 |
| RNASE12 | Ribonuclease, RNase A family, 12 (non-active) | SEQ ID NO: 11454 |
| RNASE13 | Ribonuclease, RNase A family, 13 (non-active) | SEQ ID NO: 11455 |
| RNASE2 | Ribonuclease, RNase A family, 2 (liver, eosinophil- | SEQ ID NO: 11456 |
| | derived neurotoxin) | |
| RNASE3 | Ribonuclease, RNase A family, 3 | SEQ ID NO: 11457 |
| RNASE4 | Ribonuclease, RNase A family, 4 | SEQ ID NOS: 11458-11460 |
| RNASE6 | Ribonuclease, RNase A family, k6 | SEQ ID NO: 11461 |
| RNASE7 | Ribonuclease, RNase A family, 7 | SEQ ID NOS: 11462-11463 |
| RNASE8 | Ribonuclease, RNase A family, 8 | SEQ ID NO: 11464 |
| RNASE9 | Ribonuclease, RNase A family, 9 (non-active) | SEQ ID NOS: 11465-11475 |
| RNASEH1 | Ribonuclease H1 | SEQ ID NOS: 11476-11478 |
| RNASET2 | Ribonuclease T2 | SEQ ID NOS: 11479-11486 |
| RNF146 | Ring finger protein 146 | SEQ ID NOS: 11487-11498 |
| RNF148 | Ring finger protein 148 | SEQ ID NOS: 11499-11500 |
| RNF150 | Ring finger protein 150 | SEQ ID NOS: 11501-11505 |
| RNF167 | Ring finger protein 167 | SEQ ID NOS: 11506-11516 |
| RNF220 | Ring finger protein 220 | SEQ ID NOS: 11517-11523 |
| RNF34 | Ring finger protein 34, E3 ubiquitin protein ligase | SEQ ID NOS: 11524-11531 |
| RNLS | Renalase, FAD-dependent amine oxidase | SEQ ID NOS: 11532-11534 |
| RNPEP | Arginyl aminopeptidase (aminopeptidase B) | SEQ ID NOS: 11535-11540 |
| ROR1 | Receptor tyrosine kinase-like orphan receptor 1 | SEQ ID NOS: 11541-11543 |
| RPL3 | Ribosomal protein L3 | SEQ ID NOS: 11544-11549 |
| RPLP2 | Ribosomal protein, large, P2 | SEQ ID NOS: 11550-11552 |
| RPN2 | Ribophorin II | SEQ ID NOS: 11553-11559 |
| RPS27L | Ribosomal protein S27-like | SEQ ID NOS: 11560-11565 |
| RS1 | Retinoschisin 1 | SEQ ID NO: 11566 |
| RSF1 | Remodeling and spacing factor 1 | SEQ ID NOS: 11567-11573 |
| RSPO1 | R-spondin 1 | SEQ ID NOS: 11574-11577 |
| RSPO2 | R-spondin 2 | SEQ ID NOS: 11578-11585 |
| RSPO3 | R-spondin 3 | SEQ ID NOS: 11586-11587 |
| RSPO4 | R-spondin 4 | SEQ ID NOS: 11588-11589 |
| RSPRY1 | Ring finger and SPRY domain containing 1 | SEQ ID NOS: 11590-11596 |
| RTBDN | Retbindin | SEQ ID NOS: 11597-11609 |
| RTN4RL1 | Reticulon 4 receptor-like 1 | SEQ ID NO: 11610 |
| RTN4RL2 | Reticulon 4 receptor-like 2 | SEQ ID NOS: 11611-11613 |
| SAA1 | Serum amyloid A1 | SEQ ID NOS: 11614-11616 |
| SAA2 | Serum amyloid A2 | SEQ ID NOS: 11617-11622 |
| SAA4 | Serum amyloid A4, constitutive | SEQ ID NO: 11623 |
| SAP30 | Sin3A-associated protein, 30 kDa | SEQ ID NO: 11624 |
| SAR1A | Secretion associated, Ras related GTPase 1A | SEQ ID NOS: 11625-11631 |
| SARAF | Store-operated calcium entry-associated regulatory | SEQ ID NOS: 11632-11642 |
| | factor | |
| SARM1 | Sterile alpha and TIR motif containing 1 | SEQ ID NOS: 11643-11646 |
| SATB1 | SATB homeobox 1 | SEQ ID NOS: 11647-11659 |
| SAXO2 | Stabilizer of axonemal microtubules 2 | SEQ ID NOS: 11660-11664 |
| SBSN | Suprabasin | SEQ ID NOS: 11665-11667 |
| SBSPON | Somatomedin B and thrombospondin, type 1 | SEQ ID NO: 11668 |
| | domain containing | |
| SCARF1 | Scavenger receptor class F, member 1 | SEQ ID NOS: 11669-11673 |
| SCG2 | Secretogranin II | SEQ ID NOS: 11674-11676 |
| SCG3 | Secretogranin III | SEQ ID NOS: 11677-11679 |
| SCG5 | Secretogranin V | SEQ ID NOS: 11680-11684 |
| SCGB1A1 | Secretoglobin, family 1A, member 1 (uteroglobin) | SEQ ID NOS: 11685-11686 |
| SCGB1C1 | Secretoglobin, family 1C, member 1 | SEQ ID NO: 11687 |
| SCGB1C2 | Secretoglobin, family 1C, member 2 | SEQ ID NO: 11688 |
| SCGB1D1 | Secretoglobin, family 1D, member 1 | SEQ ID NO: 11689 |
| SCGB1D2 | Secretoglobin, family 1D, member 2 | SEQ ID NO: 11690 |
| SCGB1D4 | Secretoglobin, family 1D, member 4 | SEQ ID NO: 11691 |
| SCGB2A1 | Secretoglobin, family 2A, member 1 | SEQ ID NO: 11692 |
| SCGB2A2 | Secretoglobin, family 2A, member 2 | SEQ ID NOS: 11693-11694 |
| SCGB2B2 | Secretoglobin, family 2B, member 2 | SEQ ID NOS: 11695-11696 |
| SCGB3A1 | Secretoglobin, family 3A, member 1 | SEQ ID NO: 11697 |
| SCGB3A2 | Secretoglobin, family 3A, member 2 | SEQ ID NOS: 11698-11699 |
| SCN1B | Sodium channel, voltage gated, type I beta subunit | SEQ ID NOS: 11700-11705 |
| SCN3B | Sodium channel, voltage gated, type III beta subunit | SEQ ID NOS: 11706-11710 |
| SCPEP1 | Serine carboxypeptidase 1 | SEQ ID NOS: 11711-11718 |
| SCRG1 | Stimulator of chondrogenesis 1 | SEQ ID NOS: 11719-11720 |
| SCT | Secretin | SEQ ID NO: 11721 |
| SCUBE1 | Signal peptide, CUB domain, EGF-like 1 | SEQ ID NOS: 11722-11725 |
| SCUBE2 | Signal peptide, CUB domain, EGF-like 2 | SEQ ID NOS: 11726-11732 |
| SCUBE3 | Signal peptide, CUB domain, EGF-like 3 | SEQ ID NO: 11733 |
| SDC1 | Syndecan 1 | SEQ ID NOS: 11734-11738 |
| SDF2 | Stromal cell-derived factor 2 | SEQ ID NOS: 11739-11741 |
| SDF2L1 | Stromal cell-derived factor 2-like 1 | SEQ ID NO: 11742 |
| SDF4 | Stromal cell derived factor 4 | SEQ ID NOS: 11743-11746 |
| SDHAF2 | Succinate dehydrogenase complex assembly factor 2 | SEQ ID NOS: 11747-11754 |
| SDHAF4 | Succinate dehydrogenase complex assembly factor 4 | SEQ ID NO: 11755 |
| SDHB | Succinate dehydrogenase complex, subunit B, iron | SEQ ID NOS: 11756-11758 |
| | sulfur (Ip) | |
| SDHD | Succinate dehydrogenase complex, subunit D, | SEQ ID NOS: 11759-11768 |
| | integral membrane protein | |
| SEC14L3 | SEC14-like lipid binding 3 | SEQ ID NOS: 11769-11775 |
| SEC16A | SEC16 homolog A, endoplasmic reticulum export | SEQ ID NOS: 11776-11782 |
| | factor | |
| SEC16B | SEC16 homolog B, endoplasmic reticulum export | SEQ ID NOS: 11783-11786 |
| | factor | |
| SEC22C | SEC22 homolog C, vesicle trafficking protein | SEQ ID NOS: 11787-11799 |
| SEC31A | SEC31 homolog A, COPII coat complex component | SEQ ID NOS: 11800-11829 |
| SECISBP2 | SECIS binding protein 2 | SEQ ID NOS: 11830-11834 |
| SECTM1 | Secreted and transmembrane 1 | SEQ ID NOS: 11835-11842 |
| SEL1L | Sel-1 suppressor of lin-12-like (C. elegans) | SEQ ID NOS: 11843-11845 |
| SEPT15 | 15 kDa selenoprotein | SEQ ID NOS: 11846-11852 |
| SELM | Selenoprotein M | SEQ ID NOS: 11853-11855 |
| SEPN1 | Selenoprotein N, 1 | SEQ ID NOS: 11856-11859 |
| SELO | Selenoprotein O | SEQ ID NOS: 11860-11861 |
| SEPP1 | Selenoprotein P, plasma, 1 | SEQ ID NOS: 11862-11867 |
| SEMA3A | Sema domain, immunoglobulin domain (Ig), short | SEQ ID NOS: 11868-11872 |
| | basic domain, secreted, (semaphorin) 3A | |
| SEMA3B | Sema domain, immunoglobulin domain (Ig), short | SEQ ID NOS: 11873-11879 |
| | basic domain, secreted, (semaphorin) 3B | |
| SEMA3C | Sema domain, immunoglobulin domain (Ig), short | SEQ ID NOS: 11880-11884 |
| | basic domain, secreted, (semaphorin) 3C | |
| SEMA3E | Sema domain, immunoglobulin domain (Ig), short | SEQ ID NOS: 11885-11889 |
| | basic domain, secreted, (semaphorin) 3E | |
| SEMA3F | Sema domain, immunoglobulin domain (Ig), short | SEQ ID NOS: 11890-11896 |
| | basic domain, secreted, (semaphorin) 3F | |
| SEMA3G | Sema domain, immunoglobulin domain (Ig), short | SEQ ID NOS: 11897-11899 |
| | basic domain, secreted, (semaphorin) 3G | |
| SEMA4A | Sema domain, immunoglobulin domain (Ig), | SEQ ID NOS: 11900-11908 |
| | transmembrane domain (TM) and short cytoplasmic | |
| | domain, (semaphorin) 4A | |
| SEMA4B | Sema domain, immunoglobulin domain (Ig), | SEQ ID NOS: 11909-11919 |
| | transmembrane domain (TM) and short cytoplasmic | |
| | domain, (semaphorin) 4B | |
| SEMA4C | Sema domain, immunoglobulin domain (Ig), | SEQ ID NOS: 11920-11922 |
| | transmembrane domain (TM) and short cytoplasmic | |
| | domain, (semaphorin) 4C | |
| SEMA4D | Sema domain, immunoglobulin domain (Ig), | SEQ ID NOS: 11923-11936 |
| | transmembrane domain (TM) and short cytoplasmic | |
| | domain, (semaphorin) 4D | |
| SEMA4F | Sema domain, immunoglobulin domain (Ig), | SEQ ID NOS: 11937-11945 |
| | transmembrane domain (TM) and short cytoplasmic | |
| | domain, (semaphorin) 4F | |
| SEMA4G | Sema domain, immunoglobulin domain (Ig), | SEQ ID NOS: 11946-11953 |
| | transmembrane domain (TM) and short cytoplasmic | |
| | domain, (semaphorin) 4G | |
| SEMA5A | Sema domain, seven thrombospondin repeats (type | SEQ ID NOS: 11954-11955 |
| | 1 and type 1-like), transmembrane domain (TM) and | |
| | short cytoplasmic domain, (semaphorin) 5A | |
| SEMA6A | Sema domain, transmembrane domain (TM), and | SEQ ID NOS: 11956-11963 |
| | cytoplasmic domain, (semaphorin) 6A | |
| SEMA6C | Sema domain, transmembrane domain (TM), and | SEQ ID NOS: 11964-11969 |
| | cytoplasmic domain, (semaphorin) 6C | |
| SEMA6D | Sema domain, transmembrane domain (TM), and | SEQ ID NOS: 11970-11983 |
| | cytoplasmic domain, (semaphorin) 6D | |
| SEMG1 | Semenogelin I | SEQ ID NO: 11984 |
| SEMG2 | Semenogelin II | SEQ ID NO: 11985 |
| SEPT9 | Septin 9 | SEQ ID NOS: 11986-12022 |
| SERPINA1 | Serpin peptidase inhibitor, clade A (alpha-1 | SEQ ID NOS: 12023-12039 |
| | antiproteinase, antitrypsin), member 1 | |
| SERPINA10 | Serpin peptidase inhibitor, clade A (alpha-1 | SEQ ID NOS: 12040-12043 |
| | antiproteinase, antitrypsin), member 10 | |
| SERPINA11 | Serpin peptidase inhibitor, clade A (alpha-1 | SEQ ID NO: 12044 |
| | antiproteinase, antitrypsin), member 11 | |
| SERPINA12 | Serpin peptidase inhibitor, clade A (alpha-1 | SEQ ID NOS: 12045-12046 |
| | antiproteinase, antitrypsin), member 12 | |
| SERPINA3 | Serpin peptidase inhibitor, clade A (alpha-1 | SEQ ID NOS: 12047-12053 |
| | antiproteinase, antitrypsin), member 3 | |
| SERPINA4 | Serpin peptidase inhibitor, clade A (alpha-1 | SEQ ID NOS: 12054-12056 |
| | antiproteinase, antitrypsin), member 4 | |
| SERPINA5 | Serpin peptidase inhibitor, clade A (alpha-1 | SEQ ID NOS: 12057-12068 |
| | antiproteinase, antitrypsin), member 5 | |
| SERPINA6 | Serpin peptidase inhibitor, clade A (alpha-1 | SEQ ID NOS: 12069-12071 |
| | antiproteinase, antitrypsin), member 6 | |
| SERPINA7 | Serpin peptidase inhibitor, clade A (alpha-1 | SEQ ID NOS: 12072-12073 |
| | antiproteinase, antitrypsin), member 7 | |
| SERPINA9 | Serpin peptidase inhibitor, clade A (alpha-1 | SEQ ID NOS: 12074-12080 |
| | antiproteinase, antitrypsin), member 9 | |
| SERPINB2 | Serpin peptidase inhibitor, clade B (ovalbumin), | SEQ ID NOS: 12081-12085 |
| | member 2 | |
| SERPINC1 | Serpin peptidase inhibitor, clade C (antithrombin), | SEQ ID NOS: 12086-12087 |
| | member 1 | |
| SERPIND1 | Serpin peptidase inhibitor, clade D (heparin | SEQ ID NOS: 12088-12089 |
| | cofactor), member 1 | |
| SERPINE1 | Serpin peptidase inhibitor, clade E (nexin, | SEQ ID NO: 12090 |
| | plasminogen activator inhibitor type 1), member 1 | |
| SERPINE2 | Serpin peptidase inhibitor, clade E (nexin, | SEQ ID NOS: 12091-12097 |
| | plasminogen activator inhibitor type 1), member 2 | |
| SERPINE3 | Serpin peptidase inhibitor, clade E (nexin, | SEQ ID NOS: 12098-12101 |
| | plasminogen activator inhibitor type 1), member 3 | |
| SERPINF1 | Serpin peptidase inhibitor, clade F (alpha-2 | SEQ ID NOS: 12102-12110 |
| | antiplasmin, pigment epithelium derived factor), | |
| | member 1 | |
| SERPINF2 | Serpin peptidase inhibitor, clade F (alpha-2 | SEQ ID NOS: 12111-12115 |
| | antiplasmin, pigment epithelium derived factor), | |
| | member 2 | |
| SERPING1 | Serpin peptidase inhibitor, clade G (C1 inhibitor), | SEQ ID NOS: 12116-12126 |
| | member 1 | |
| SERPINH1 | Serpin peptidase inhibitor, clade H (heat shock | SEQ ID NOS: 12127-12141 |
| | protein 47), member 1, (collagen binding protein 1) | |
| SERPINI1 | Serpin peptidase inhibitor, clade I (neuroserpin), | SEQ ID NOS: 12142-12146 |
| | member 1 | |
| SERPINI2 | Serpin peptidase inhibitor, clade I (pancpin), | SEQ ID NOS: 12147-12153 |
| | member 2 | |
| SEZ6L2 | Seizure related 6 homolog (mouse)-like 2 | SEQ ID NOS: 12154-12160 |
| SFRP1 | Secreted frizzled-related protein 1 | SEQ ID NOS: 12161-12162 |
| SFRP2 | Secreted frizzled-related protein 2 | SEQ ID NO: 12163 |
| SFRP4 | Secreted frizzled-related protein 4 | SEQ ID NOS: 12164-12165 |
| SFRP5 | Secreted frizzled-related protein 5 | SEQ ID NO: 12166 |
| SFTA2 | Surfactant associated 2 | SEQ ID NOS: 12167-12168 |
| SFTPA1 | Surfactant protein A1 | SEQ ID NOS: 12169-12173 |
| SFTPA2 | Surfactant protein A2 | SEQ ID NOS: 12174-12178 |
| SFTPB | Surfactant protein B | SEQ ID NOS: 12179-12183 |
| SFTPD | Surfactant protein D | SEQ ID NOS: 12184-12185 |
| SFXN5 | Sideroflexin 5 | SEQ ID NOS: 12186-12190 |
| SGCA | Sarcoglycan, alpha (50 kDa dystrophin-associated | SEQ ID NOS: 12191-12198 |
| | glycoprotein) | |
| SGSH | N-sulfoglucosamine sulfohydrolase | SEQ ID NOS: 12199-12207 |
| SH3RF3 | SH3 domain containing ring finger 3 | SEQ ID NO: 12208 |
| SHBG | Sex hormone-binding globulin | SEQ ID NOS: 12209-12227 |
| SHE | Src homology 2 domain containing E | SEQ ID NOS: 12228-12230 |
| SHH | Sonic hedgehog | SEQ ID NOS: 12231-12234 |
| SHKBP1 | SH3KBP1 binding protein 1 | SEQ ID NOS: 12235-12250 |
| SIAE | Sialic acid acetylesterase | SEQ ID NOS: 12251-12253 |
| SIDT2 | SID1 transmembrane family, member 2 | SEQ ID NOS: 12254-12263 |
| SIGLEC10 | Sialic acid binding Ig-like lectin 10 | SEQ ID NOS: 12264-12272 |
| SIGLEC6 | Sialic acid binding Ig-like lectin 6 | SEQ ID NOS: 12273-12278 |
| SIGLEC7 | Sialic acid binding Ig-like lectin 7 | SEQ ID NOS: 12279-12283 |
| SIGLECL1 | SIGLEC family like 1 | SEQ ID NOS: 12284-12289 |
| SIGMAR1 | Sigma non-opioid intracellular receptor 1 | SEQ ID NOS: 12290-12293 |
| SIL1 | SIL1 nucleotide exchange factor | SEQ ID NOS: 12294-12302 |
| SIRPB1 | Signal-regulatory protein beta 1 | SEQ ID NOS: 12303-12315 |
| SIRPD | Signal-regulatory protein delta | SEQ ID NOS: 12316-12318 |
| SLAMF1 | Signaling lymphocytic activation molecule family | SEQ ID NOS: 12319-12321 |
| | member 1 | |
| SLAMF7 | SLAM family member 7 | SEQ ID NOS: 12322-12330 |
| SLC10A3 | Solute carrier family 10, member 3 | SEQ ID NOS: 12331-12335 |
| SLC15A3 | Solute carrier family 15 (oligopeptide transporter), | SEQ ID NOS: 12336-12341 |
| | member 3 | |
| SLC25A14 | Solute carrier family 25 (mitochondrial carrier, | SEQ ID NOS: 12342-12348 |
| | brain), member 14 | |
| SLC25A25 | Solute carrier family 25 (mitochondrial carrier; | SEQ ID NOS: 12349-12355 |
| | phosphate carrier), member 25 | |
| SLC2A5 | Solute carrier family 2 (facilitated glucose/fructose | SEQ ID NOS: 12356-12364 |
| | transporter), member 5 | |
| SLC35E3 | Solute carrier family 35, member E3 | SEQ ID NOS: 12365-12366 |
| SLC39A10 | Solute carrier family 39 (zinc transporter), | SEQ ID NOS: 12367-12373 |
| | member 10 | |
| SLC39A14 | Solute carrier family 39 (zinc transporter), | SEQ ID NOS: 12374-12384 |
| | member 14 | |
| SLC39A4 | Solute carrier family 39 (zinc transporter), member 4 | SEQ ID NOS: 12385-12387 |
| SLC39A5 | Solute carrier family 39 (zinc transporter), member 5 | SEQ ID NOS: 12388-12394 |
| SLC3A1 | Solute carrier family 3 (amino acid transporter heavy | SEQ ID NOS: 12395-12404 |
| | chain), member 1 | |
| SLC51A | Solute carrier family 51, alpha subunit | SEQ ID NOS: 12405-12409 |
| SLC52A2 | Solute carrier family 52 (riboflavin transporter), | SEQ ID NOS: 12410-12420 |
| | member 2 | |
| SLC5A6 | Solute carrier family 5 (sodium/multivitamin and | SEQ ID NOS: 12421-12431 |
| | iodide cotransporter), member 6 | |
| SLC6A9 | Solute carrier family 6 (neurotransmitter | SEQ ID NOS: 12432-12439 |
| | transporter, glycine), member 9 | |
| SLC8A1 | Solute carrier family 8 (sodium/calcium exchanger), | SEQ ID NOS: 12440-12451 |
| | member 1 | |
| SLC8B1 | Solute carrier family 8 (sodium/lithium/calcium | SEQ ID NOS: 12452-12462 |
| | exchanger), member B1 | |
| SLC9A6 | Solute carrier family 9, subfamily A (NHE6, cation | SEQ ID NOS: 12463-12474 |
| | proton antiporter 6), member 6 | |
| SLCO1A2 | Solute carrier organic anion transporter family, | SEQ ID NOS: 12475-12488 |
| | member 1A2 | |
| SLIT1 | Slit guidance ligand 1 | SEQ ID NOS: 12489-12492 |
| SLIT2 | Slit guidance ligand 2 | SEQ ID NOS: 12493-12501 |
| SLIT3 | Slit guidance ligand 3 | SEQ ID NOS: 12502-12504 |
| SLITRK3 | SLIT and NTRK-like family, member 3 | SEQ ID NOS: 12505-12507 |
| SLPI | Secretory leukocyte peptidase inhibitor | SEQ ID NO: 12508 |
| SLTM | SAFB-like, transcription modulator | SEQ ID NOS: 12509-12522 |
| SLURP1 | Secreted LY6/PLAUR domain containing 1 | SEQ ID NO: 12523 |
| SMARCA2 | SWI/SNF related, matrix associated, actin dependent | SEQ ID NOS: 12524-12571 |
| | regulator of chromatin, subfamily a, member 2 | |
| SMG6 | SMG6 nonsense mediated mRNA decay factor | SEQ ID NOS: 12572-12583 |
| SMIM7 | Small integral membrane protein 7 | SEQ ID NOS: 12584-12600 |
| SMOC1 | SPARC related modular calcium binding 1 | SEQ ID NOS: 12601-12602 |
| SMOC2 | SPARC related modular calcium binding 2 | SEQ ID NOS: 12603-12607 |
| SMPDL3A | Sphingomyelin phosphodiesterase, acid-like 3A | SEQ ID NOS: 12608-12609 |
| SMPDL3B | Sphingomyelin phosphodiesterase, acid-like 3B | SEQ ID NOS: 12610-12614 |
| SMR3A | Submaxillary gland androgen regulated protein 3A | SEQ ID NO: 12615 |
| SMR3B | Submaxillary gland androgen regulated protein 3B | SEQ ID NOS: 12616-12618 |
| SNED1 | Sushi, nidogen and EGF-like domains 1 | SEQ ID NOS: 12619-12625 |
| SNTB1 | Syntrophin, beta 1 (dystrophin-associated protein | SEQ ID NOS: 12626-12628 |
| | A1, 59 kDa, basic component 1) | |
| SNTB2 | Syntrophin, beta 2 (dystrophin-associated protein | SEQ ID NOS: 12629-12633 |
| | A1, 59 kDa, basic component 2) | |
| SNX14 | Sorting nexin 14 | SEQ ID NOS: 12634-12645 |
| SOD3 | Superoxide dismutase 3, extracellular | SEQ ID NOS: 12646-12647 |
| SOST | Sclerostin | SEQ ID NO: 12648 |
| SOSTDC1 | Sclerostin domain containing 1 | SEQ ID NOS: 12649-12650 |
| SOWAHA | Sosondowah ankyrin repeat domain family member | SEQ ID NO: 12651 |
| | A | |
| SPACA3 | Sperm acrosome associated 3 | SEQ ID NOS: 12652-12654 |
| SPACA4 | Sperm acrosome associated 4 | SEQ ID NO: 12655 |
| SPACA5 | Sperm acrosome associated 5 | SEQ ID NOS: 12656-12657 |
| SPACA5B | Sperm acrosome associated 5B | SEQ ID NO: 12658 |
| SPACA7 | Sperm acrosome associated 7 | SEQ ID NOS: 12659-12662 |
| SPAG11A | Sperm associated antigen 11A | SEQ ID NOS: 12663-12671 |
| SPAG11B | Sperm associated antigen 11B | SEQ ID NOS: 12672-12680 |
| SPARC | Secreted protein, acidic, cysteine-rich (osteonectin) | SEQ ID NOS: 12681-12685 |
| SPARCL1 | SPARC-like 1 (hevin) | SEQ ID NOS: 12686-12695 |
| SPATA20 | Spermatogenesis associated 20 | SEQ ID NOS: 12696-12709 |
| SPESP1 | Sperm equatorial segment protein 1 | SEQ ID NO: 12710 |
| SPINK1 | Serine peptidase inhibitor, Kazal type 1 | SEQ ID NOS: 12711-12712 |
| SPINK13 | Serine peptidase inhibitor, Kazal type 13 (putative) | SEQ ID NOS: 12713-12715 |
| SPINK14 | Serine peptidase inhibitor, Kazal type 14 (putative) | SEQ ID NOS: 12716-12717 |
| SPINK2 | Serine peptidase inhibitor, Kazal type 2 (acrosin- | SEQ ID NOS: 12718-12723 |
| | trypsin inhibitor) | |
| SPINK4 | Serine peptidase inhibitor, Kazal type 4 | SEQ ID NOS: 12724-12725 |
| SPINK5 | Serine peptidase inhibitor, Kazal type 5 | SEQ ID NOS: 12726-12731 |
| SPINK6 | Serine peptidase inhibitor, Kazal type 6 | SEQ ID NOS: 12732-12734 |
| SPINK7 | Serine peptidase inhibitor, Kazal type 7 (putative) | SEQ ID NOS: 12735-12736 |
| SPINK8 | Serine peptidase inhibitor, Kazal type 8 (putative) | SEQ ID NO: 12737 |
| SPINK9 | Serine peptidase inhibitor, Kazal type 9 | SEQ ID NOS: 12738-12739 |
| SPINT1 | Serine peptidase inhibitor, Kunitz type 1 | SEQ ID NOS: 12740-12747 |
| SPINT2 | Serine peptidase inhibitor, Kunitz type, 2 | SEQ ID NOS: 12748-12755 |
| SPINT3 | Serine peptidase inhibitor, Kunitz type, 3 | SEQ ID NO: 12756 |
| SPINT4 | Serine peptidase inhibitor, Kunitz type 4 | SEQ ID NO: 12757 |
| SPOCK1 | Sparc/osteonectin, cwcv and kazal-like domains | SEQ ID NOS: 12758-12761 |
| | proteoglycan (testican) 1 | |
| SPOCK2 | Sparc/osteonectin, cwcv and kazal-like domains | SEQ ID NOS: 12762-12765 |
| | proteoglycan (testican) 2 | |
| SPOCK3 | Sparc/osteonectin, cwcv and kazal-like domains | SEQ ID NOS: 12766-12791 |
| | proteoglycan (testican) 3 | |
| SPON1 | Spondin 1, extracellular matrix protein | SEQ ID NO: 12792 |
| SPON2 | Spondin 2, extracellular matrix protein | SEQ ID NOS: 12793-12802 |
| SPP1 | Secreted phosphoprotein 1 | SEQ ID NOS: 12803-12807 |
| SPP2 | Secreted phosphoprotein 2, 24 kDa | SEQ ID NOS: 12808-12810 |
| SPRN | Shadow of prion protein homolog (zebrafish) | SEQ ID NO: 12811 |
| SPRYD3 | SPRY domain containing 3 | SEQ ID NOS: 12812-12815 |
| SPRYD4 | SPRY domain containing 4 | SEQ ID NO: 12816 |
| SPTY2D1- | SPTY2D1 antisense RNA 1 | SEQ ID NOS: 12817-12822 |
| AS1 | | |
| SPX | Spexin hormone | SEQ ID NOS: 12823-12824 |
| SRGN | Serglycin | SEQ ID NO: 12825 |
| SRL | Sarcalumenin | SEQ ID NOS: 12826-12828 |
| SRP14 | Signal recognition particle 14 kDa (homologous Alu | SEQ ID NOS: 12829-12832 |
| | RNA binding protein) | |
| SRPX | Sushi-repeat containing protein, X-linked | SEQ ID NOS: 12833-12836 |
| SRPX2 | Sushi-repeat containing protein, X-linked 2 | SEQ ID NOS: 12837-12840 |
| SSC4D | Scavenger receptor cysteine rich family, 4 domains | SEQ ID NO: 12841 |
| SSC5D | Scavenger receptor cysteine rich family, 5 domains | SEQ ID NOS: 12842-12845 |
| SSPO | SCO-spondin | SEQ ID NO: 12846 |
| SSR2 | Signal sequence receptor, beta (translocon- | SEQ ID NOS: 12847-12856 |
| | associated protein beta) | |
| SST | Somatostatin | SEQ ID NO: 12857 |
| ST3GAL1 | ST3 beta-galactoside alpha-2,3-sialyltransferase 1 | SEQ ID NOS: 12858-12865 |
| ST3GAL4 | ST3 beta-galactoside alpha-2,3-sialyltransferase 4 | SEQ ID NOS: 12866-12881 |
| ST6GAL1 | ST6 beta-galactosamide alpha-2,6-sialyltranferase 1 | SEQ ID NOS: 12882-12897 |
| ST6GALNAC | ST6 (alpha-N-acetyl-neuraminyl-2,3-beta-galactosyl- | SEQ ID NOS: 12898-12902 |
| 2 | 1,3)-N-acetylgalactosaminide alpha-2,6- | |
| | sialyltransferase 2 | |
| ST6GALNAC | ST6 (alpha-N-acetyl-neuraminyl-2,3-beta-galactosyl- | SEQ ID NOS: 12903-12904 |
| 5 | 1,3)-N-acetylgalactosaminide alpha-2,6- | |
| | sialyltransferase 5 | |
| ST6GALNAC | ST6 (alpha-N-acetyl-neuraminyl-2,3-beta-galactosyl- | SEQ ID NOS: 12905-12912 |
| 6 | 1,3)-N-acetylgalactosaminide alpha-2,6- | |
| | sialyltransferase 6 | |
| ST8SIA2 | ST8 alpha-N-acetyl-neuraminide alpha-2,8- | SEQ ID NOS: 12913-12915 |
| | sialyltransferase 2 | |
| ST8SIA4 | ST8 alpha-N-acetyl-neuraminide alpha-2,8- | SEQ ID NOS: 12916-12918 |
| | sialyltransferase 4 | |
| ST8SIA6 | ST8 alpha-N-acetyl-neuraminide alpha-2,8- | SEQ ID NOS: 12919-12920 |
| | sialyltransferase 6 | |
| STARD7 | StAR-related lipid transfer (START) domain | SEQ ID NOS: 12921-12922 |
| | containing 7 | |
| STATH | Statherin | SEQ ID NOS: 12923-12925 |
| STC1 | Stanniocalcin 1 | SEQ ID NOS: 12926-12927 |
| STC2 | Stanniocalcin 2 | SEQ ID NOS: 12928-12930 |
| STMND1 | Stathmin domain containing 1 | SEQ ID NOS: 12931-12932 |
| C7orf73 | Chromosome 7 open reading frame 73 | SEQ ID NOS: 12933-12934 |
| STOML2 | Stomatin (EPB72)-like 2 | SEQ ID NOS: 12935-12938 |
| STOX1 | Storkhead box 1 | SEQ ID NOS: 12939-12943 |
| STRC | Stereocilin | SEQ ID NOS: 12944-12949 |
| SUCLG1 | Succinate-CoA ligase, alpha subunit | SEQ ID NOS: 12950-12951 |
| SUDS3 | SDS3 homolog, SIN3A corepressor complex | SEQ ID NO: 12952 |
| | component | |
| SULF1 | Sulfatase 1 | SEQ ID NOS: 12953-12963 |
| SULF2 | Sulfatase 2 | SEQ ID NOS: 12964-12968 |
| SUMF1 | Sulfatase modifying factor 1 | SEQ ID NOS: 12969-12973 |
| SUMF2 | Sulfatase modifying factor 2 | SEQ ID NOS: 12974-12987 |
| SUSD1 | Sushi domain containing 1 | SEQ ID NOS: 12988-12993 |
| SUSD5 | Sushi domain containing 5 | SEQ ID NOS: 12994-12995 |
| SVEP1 | Sushi, von Willebrand factor type A, EGF and | SEQ ID NOS: 12996-12998 |
| | pentraxin domain containing 1 | |
| SWSAP1 | SWIM-type zinc finger 7 associated protein 1 | SEQ ID NO: 12999 |
| SYAP1 | Synapse associated protein 1 | SEQ ID NO: 13000 |
| SYCN | Syncollin | SEQ ID NO: 13001 |
| TAC1 | Tachykinin, precursor 1 | SEQ ID NOS: 13002-13004 |
| TAC3 | Tachykinin 3 | SEQ ID NOS: 13005-13014 |
| TAC4 | Tachykinin 4 (hemokinin) | SEQ ID NOS: 13015-13020 |
| TAGLN2 | Transgelin 2 | SEQ ID NOS: 13021-13024 |
| TAPBP | TAP binding protein (tapasin) | SEQ ID NOS: 13025-13030 |
| TAPBPL | TAP binding protein-like | SEQ ID NOS: 13031-13032 |
| TBL2 | Transducin (beta)-like 2 | SEQ ID NOS: 13033-13045 |
| TBX10 | T-box 10 | SEQ ID NO: 13046 |
| TCF12 | Transcription factor 12 | SEQ ID NOS: 13047-13060 |
| TCN1 | Transcobalamin I (vitamin B12 binding protein, R | SEQ ID NO: 13061 |
| | binder family) | |
| TCN2 | Transcobalamin II | SEQ ID NOS: 13062-13065 |
| TCTN1 | Tectonic family member 1 | SEQ ID NOS: 13066-13084 |
| TCTN3 | Tectonic family member 3 | SEQ ID NOS: 13085-13089 |
| TDP2 | Tyrosyl-DNA phosphodiesterase 2 | SEQ ID NOS: 13090-13091 |
| C14orf80 | Chromosome 14 open reading frame 80 | SEQ ID NOS: 13092-13105 |
| TEK | TEK tyrosine kinase, endothelial | SEQ ID NOS: 13106-13110 |
| TEPP | Testis, prostate and placenta expressed | SEQ ID NOS: 13111-13112 |
| TEX101 | Testis expressed 101 | SEQ ID NOS: 13113-13114 |
| TEX264 | Testis expressed 264 | SEQ ID NOS: 13115-13126 |
| C1orf234 | Chromosome 1 open reading frame 234 | SEQ ID NOS: 13127-13129 |
| TF | Transferrin | SEQ ID NOS: 13130-13136 |
| TFAM | Transcription factor A, mitochondrial | SEQ ID NOS: 13137-13139 |
| TFF1 | Trefoil factor 1 | SEQ ID NO: 13140 |
| TFF2 | Trefoil factor 2 | SEQ ID NO: 13141 |
| TFF3 | Trefoil factor 3 (intestinal) | SEQ ID NOS: 13142-13144 |
| TFPI | Tissue factor pathway inhibitor (lipoprotein- | SEQ ID NOS: 13145-13154 |
| | associated coagulation inhibitor) | |
| TFPI2 | Tissue factor pathway inhibitor 2 | SEQ ID NOS: 13155-13156 |
| TG | Thyroglobulin | SEQ ID NOS: 13157-13166 |
| TGFB1 | Transforming growth factor, beta 1 | SEQ ID NOS: 13167-13168 |
| TGFB2 | Transforming growth factor, beta 2 | SEQ ID NOS: 13169-13170 |
| TGFB3 | Transforming growth factor, beta 3 | SEQ ID NOS: 13171-13172 |
| TGFBI | Transforming growth factor, beta-induced, 68 kDa | SEQ ID NOS: 13173-13180 |
| TGFBR1 | Transforming growth factor, beta receptor 1 | SEQ ID NOS: 13181-13190 |
| TGFBR3 | Transforming growth factor, beta receptor III | SEQ ID NOS: 13191-13197 |
| THBS1 | Thrombospondin 1 | SEQ ID NOS: 13198-13199 |
| THBS2 | Thrombospondin 2 | SEQ ID NOS: 13200-13202 |
| THBS3 | Thrombospondin 3 | SEQ ID NOS: 13203-13207 |
| THBS4 | Thrombospondin 4 | SEQ ID NOS: 13208-13209 |
| THOC3 | THO complex 3 | SEQ ID NOS: 13210-13219 |
| THPO | Thrombopoietin | SEQ ID NOS: 13220-13225 |
| THSD4 | Thrombospondin, type I, domain containing 4 | SEQ ID NOS: 13226-13229 |
| THY1 | Thy-1 cell surface antigen | SEQ ID NOS: 13230-13235 |
| TIE1 | Tyrosine kinase with immunoglobulin-like and EGF- | SEQ ID NOS: 13236-13237 |
| | like domains 1 | |
| TIMMDC1 | Translocase of inner mitochondrial membrane | SEQ ID NOS: 13238-13245 |
| | domain containing 1 | |
| TIMP1 | TIMP metallopeptidase inhibitor 1 | SEQ ID NOS: 13246-13250 |
| TIMP2 | TIMP metallopeptidase inhibitor 2 | SEQ ID NOS: 13251-13255 |
| TIMP3 | TIMP metallopeptidase inhibitor 3 | SEQ ID NO: 13256 |
| TIMP4 | TIMP metallopeptidase inhibitor 4 | SEQ ID NO: 13257 |
| TINAGL1 | Tubulointerstitial nephritis antigen-like 1 | SEQ ID NOS: 13258-13260 |
| TINF2 | TERF1 (TRF1)-interacting nuclear factor 2 | SEQ ID NOS: 13261-13270 |
| TLL2 | Tolloid-like 2 | SEQ ID NO: 13271 |
| TLR1 | Toll-like receptor 1 | SEQ ID NOS: 13272-13277 |
| TLR3 | Toll-like receptor 3 | SEQ ID NOS: 13278-13280 |
| TM2D2 | TM2 domain containing 2 | SEQ ID NOS: 13281-13286 |
| TM2D3 | TM2 domain containing 3 | SEQ ID NOS: 13287-13294 |
| TM7SF3 | Transmembrane 7 superfamily member 3 | SEQ ID NOS: 13295-13309 |
| TM95F1 | Transmembrane 9 superfamily member 1 | SEQ ID NOS: 13310-13320 |
| TMCO6 | Transmembrane and coiled-coil domains 6 | SEQ ID NOS: 13321-13328 |
| TMED1 | Transmembrane p24 trafficking protein 1 | SEQ ID NOS: 13329-13335 |
| TMED2 | Transmembrane p24 trafficking protein 2 | SEQ ID NOS: 13336-13338 |
| TMED3 | Transmembrane p24 trafficking protein 3 | SEQ ID NOS: 13339-13342 |
| TMED4 | Transmembrane p24 trafficking protein 4 | SEQ ID NOS: 13343-13345 |
| TMED5 | Transmembrane p24 trafficking protein 5 | SEQ ID NOS: 13346-13349 |
| TMED7 | Transmembrane p24 trafficking protein 7 | SEQ ID NOS: 13350-13351 |
| TMED7- | TMED7-TICAM2 readthrough | SEQ ID NOS: 13352-13353 |
| TICAM2 | | |
| TMEM108 | Transmembrane protein 108 | SEQ ID NOS: 13354-13362 |
| TMEM116 | Transmembrane protein 116 | SEQ ID NOS: 13363-13374 |
| TMEM119 | Transmembrane protein 119 | SEQ ID NOS: 13375-13378 |
| TMEM155 | Transmembrane protein 155 | SEQ ID NOS: 13379-13382 |
| TMEM168 | Transmembrane protein 168 | SEQ ID NOS: 13383-13388 |
| TMEM178A | Transmembrane protein 178A | SEQ ID NOS: 13389-13390 |
| TMEM179 | Transmembrane protein 179 | SEQ ID NOS: 13391-13396 |
| TMEM196 | Transmembrane protein 196 | SEQ ID NOS: 13397-13401 |
| TMEM199 | Transmembrane protein 199 | SEQ ID NOS: 13402-13405 |
| TMEM205 | Transmembrane protein 205 | SEQ ID NOS: 13406-13419 |
| TMEM213 | Transmembrane protein 213 | SEQ ID NOS: 13420-13423 |
| TMEM25 | Transmembrane protein 25 | SEQ ID NOS: 13424-13440 |
| TMEM30C | Transmembrane protein 30C | SEQ ID NO: 13441 |
| TMEM38B | Transmembrane protein 38B | SEQ ID NOS: 13442-13446 |
| TMEM44 | Transmembrane protein 44 | SEQ ID NOS: 13447-13456 |
| TMEM52 | Transmembrane protein 52 | SEQ ID NOS: 13457-13461 |
| TMEM52B | Transmembrane protein 52B | SEQ ID NOS: 13462-13464 |
| TMEM59 | Transmembrane protein 59 | SEQ ID NOS: 13465-13472 |
| TMEM67 | Transmembrane protein 67 | SEQ ID NOS: 13473-13484 |
| TMEM70 | Transmembrane protein 70 | SEQ ID NOS: 13485-13487 |
| TMEM87A | Transmembrane protein 87A | SEQ ID NOS: 13488-13497 |
| TMEM94 | Transmembrane protein 94 | SEQ ID NOS: 13498-13513 |
| TMEM95 | Transmembrane protein 95 | SEQ ID NOS: 13514-13516 |
| TMIGD1 | Transmembrane and immunoglobulin domain | SEQ ID NOS: 13517-13518 |
| | containing 1 | |
| TMPRSS12 | Transmembrane (C-terminal) protease, serine 12 | SEQ ID NOS: 13519-13520 |
| TMPRSS5 | Transmembrane protease, serine 5 | SEQ ID NOS: 13521-13532 |
| TMUB1 | Transmembrane and ubiquitin-like domain | SEQ ID NOS: 13533-13539 |
| | containing 1 | |
| TMX2 | Thioredoxin-related transmembrane protein 2 | SEQ ID NOS: 13540-13547 |
| TMX3 | Thioredoxin-related transmembrane protein 3 | SEQ ID NOS: 13548-13555 |
| TNC | Tenascin C | SEQ ID NOS: 13556-13564 |
| TNFAIP6 | Tumor necrosis factor, alpha-induced protein 6 | SEQ ID NO: 13565 |
| TNFRSF11A | Tumor necrosis factor receptor superfamily, | SEQ ID NOS: 13566-13570 |
| | member 11a, NFKB activator | |
| TNFRSF11B | Tumor necrosis factor receptor superfamily, | SEQ ID NOS: 13571-13572 |
| | member 11b | |
| TNFRSF12A | Tumor necrosis factor receptor superfamily, | SEQ ID NOS: 13573-13578 |
| | member 12A | |
| TNFRSF14 | Tumor necrosis factor receptor superfamily, | SEQ ID NOS: 13579-13585 |
| | member 14 | |
| TNFRSF18 | Tumor necrosis factor receptor superfamily, | SEQ ID NOS: 13586-13589 |
| | member 18 | |
| TNFRSF1A | Tumor necrosis factor receptor superfamily, | SEQ ID NOS: 13590-13598 |
| | member 1A | |
| TNFRSF1B | Tumor necrosis factor receptor superfamily, | SEQ ID NOS: 13599-13600 |
| | member 1B | |
| TNFRSF25 | Tumor necrosis factor receptor superfamily, | SEQ ID NOS: 13601-13612 |
| | member 25 | |
| TNFRSF6B | Tumor necrosis factor receptor superfamily, | SEQ ID NO: 13613 |
| | member 6b, decoy | |
| TNFSF11 | Tumor necrosis factor (ligand) superfamily, | SEQ ID NOS: 13614-13618 |
| | member 11 | |
| TNFSF12 | Tumor necrosis factor (ligand) superfamily, | SEQ ID NOS: 13619-13620 |
| | member 12 | |
| TNFSF12- | TNFSF12-TNFSF13 readthrough | SEQ ID NO: 13621 |
| TNFSF13 | | |
| TNFSF15 | Tumor necrosis factor (ligand) superfamily, | SEQ ID NOS: 13622-13623 |
| | member 15 | |
| TNN | Tenascin N | SEQ ID NOS: 13624-13626 |
| TNR | Tenascin R | SEQ ID NOS: 13627-13629 |
| TNXB | Tenascin XB | SEQ ID NOS: 13630-13636 |
| FAM179B | Family with sequence similarity 179, member B | SEQ ID NOS: 13637-13642 |
| TOMM7 | Translocase of outer mitochondrial membrane 7 | SEQ ID NOS: 13643-13646 |
| | homolog (yeast) | |
| TOP1MT | Topoisomerase (DNA) I, mitochondrial | SEQ ID NOS: 13647-13661 |
| TOR1A | Torsin family 1, member A (torsin A) | SEQ ID NO: 13662 |
| TOR1B | Torsin family 1, member B (torsin B) | SEQ ID NOS: 13663-13664 |
| TOR2A | Torsin family 2, member A | SEQ ID NOS: 13665-13671 |
| TOR3A | Torsin family 3, member A | SEQ ID NOS: 13672-13676 |
| TPD52 | Tumor protein D52 | SEQ ID NOS: 13677-13689 |
| TPO | Thyroid peroxidase | SEQ ID NOS: 13690-13700 |
| TPP1 | Tripeptidyl peptidase I | SEQ ID NOS: 13701-13718 |
| TPSAB1 | Tryptase alpha/beta 1 | SEQ ID NOS: 13719-13721 |
| TPSB2 | Tryptase beta 2 (gene/pseudogene) | SEQ ID NOS: 13722-13724 |
| TPSD1 | Tryptase delta 1 | SEQ ID NOS: 13725-13726 |
| TPST1 | Tyrosylprotein sulfotransferase 1 | SEQ ID NOS: 13727-13729 |
| TPST2 | Tyrosylprotein sulfotransferase 2 | SEQ ID NOS: 13730-13738 |
| TRABD2A | TraB domain containing 2A | SEQ ID NOS: 13739-13741 |
| TRABD2B | TraB domain containing 2B | SEQ ID NO: 13742 |
| TREH | Trehalase (brush-border membrane glycoprotein) | SEQ ID NOS: 13743-13745 |
| TREM1 | Triggering receptor expressed on myeloid cells 1 | SEQ ID NOS: 13746-13749 |
| TREM2 | Triggering receptor expressed on myeloid cells 2 | SEQ ID NOS: 13750-13752 |
| TRH | Thyrotropin-releasing hormone | SEQ ID NOS: 13753-13754 |
| TRIM24 | Tripartite motif containing 24 | SEQ ID NOS: 13755-13756 |
| TRIM28 | Tripartite motif containing 28 | SEQ ID NOS: 13757-13762 |
| TRIO | Trio Rho guanine nucleotide exchange factor | SEQ ID NOS: 13763-13769 |
| TRNP1 | TMF1-regulated nuclear protein 1 | SEQ ID NOS: 13770-13771 |
| TSC22D4 | TSC22 domain family, member 4 | SEQ ID NOS: 13772-13775 |
| TSHB | Thyroid stimulating hormone, beta | SEQ ID NOS: 13776-13777 |
| TSHR | Thyroid stimulating hormone receptor | SEQ ID NOS: 13778-13785 |
| TSKU | Tsukushi, small leucine rich proteoglycan | SEQ ID NOS: 13786-13790 |
| TSLP | Thymic stromal lymphopoietin | SEQ ID NOS: 13791-13793 |
| TSPAN3 | Tetraspanin 3 | SEQ ID NOS: 13794-13799 |
| TSPAN31 | Tetraspanin 31 | SEQ ID NOS: 13800-13806 |
| TSPEAR | Thrombospondin-type laminin G domain and EAR | SEQ ID NOS: 13807-13810 |
| | repeats | |
| TTC13 | Tetratricopeptide repeat domain 13 | SEQ ID NOS: 13811-13817 |
| TTC19 | Tetratricopeptide repeat domain 19 | SEQ ID NOS: 13818-13823 |
| TTC9B | Tetratricopeptide repeat domain 9B | SEQ ID NO: 13824 |
| TTLL11 | Tubulin tyrosine ligase-like family member 11 | SEQ ID NOS: 13825-13829 |
| TTR | Transthyretin | SEQ ID NOS: 13830-13832 |
| TWSG1 | Twisted gastrulation BMP signaling modulator 1 | SEQ ID NOS: 13833-13835 |
| TXNDC12 | Thioredoxin domain containing 12 (endoplasmic | SEQ ID NOS: 13836-13838 |
| | reticulum) | |
| TXNDC15 | Thioredoxin domain containing 15 | SEQ ID NOS: 13839-13845 |
| TXNDC5 | Thioredoxin domain containing 5 (endoplasmic | SEQ ID NOS: 13846-13847 |
| | reticulum) | |
| TXNRD2 | Thioredoxin reductase 2 | SEQ ID NOS: 13848-13860 |
| TYRP1 | Tyrosinase-related protein 1 | SEQ ID NOS: 13861-13863 |
| UBAC2 | UBA domain containing 2 | SEQ ID NOS: 13864-13868 |
| UBALD1 | UBA-like domain containing 1 | SEQ ID NOS: 13869-13877 |
| UBAP2 | Ubiquitin associated protein 2 | SEQ ID NOS: 13878-13884 |
| UBXN8 | UBX domain protein 8 | SEQ ID NOS: 13885-13891 |
| UCMA | Upper zone of growth plate and cartilage matrix | SEQ ID NOS: 13892-13893 |
| | associated | |
| UCN | Urocortin | SEQ ID NO: 13894 |
| UCN2 | Urocortin 2 | SEQ ID NO: 13895 |
| UCN3 | Urocortin 3 | SEQ ID NO: 13896 |
| UGGT2 | UDP-glucose glycoprotein glucosyltransferase 2 | SEQ ID NOS: 13897-13902 |
| UGT1A10 | UDP glucuronosyltransferase 1 family, polypeptide | SEQ ID NOS: 13903-13904 |
| | A10 | |
| UGT2A1 | UDP glucuronosyltransferase 2 family, polypeptide | SEQ ID NOS: 13905-13909 |
| | A1, complex locus | |
| UGT2B11 | UDP glucuronosyltransferase 2 family, polypeptide | SEQ ID NO: 13910 |
| | B11 | |
| UGT2B28 | UDP glucuronosyltransferase 2 family, polypeptide | SEQ ID NOS: 13911-13912 |
| | B28 | |
| UGT2B4 | UDP glucuronosyltransferase 2 family, polypeptide | SEQ ID NOS: 13913-13916 |
| | B4 | |
| UGT2B7 | UDP glucuronosyltransferase 2 family, polypeptide | SEQ ID NOS: 13917-13920 |
| | B7 | |
| UGT3A1 | UDP glycosyltransferase 3 family, polypeptide A1 | SEQ ID NOS: 13921-13926 |
| UGT3A2 | UDP glycosyltransferase 3 family, polypeptide A2 | SEQ ID NOS: 13927-13930 |
| UGT8 | UDP glycosyltransferase 8 | SEQ ID NOS: 13931-13933 |
| ULBP3 | UL16 binding protein 3 | SEQ ID NOS: 13934-13935 |
| UMOD | Uromodulin | SEQ ID NOS: 13936-13947 |
| UNC5C | Unc-5 netrin receptor C | SEQ ID NOS: 13948-13952 |
| UPK3B | Uroplakin 3B | SEQ ID NOS: 13953-13955 |
| USP11 | Ubiquitin specific peptidase 11 | SEQ ID NOS: 13956-13959 |
| USP14 | Ubiquitin specific peptidase 14 (tRNA-guanine | SEQ ID NOS: 13960-13966 |
| | transglycosylase) | |
| USP3 | Ubiquitin specific peptidase 3 | SEQ ID NOS: 13967-13982 |
| CIRH1A | Cirrhosis, autosomal recessive 1A (cirhin) | SEQ ID NOS: 13983-13992 |
| UTS2 | Urotensin 2 | SEQ ID NOS: 13993-13995 |
| UTS2B | Urotensin 2B | SEQ ID NOS: 13996-14001 |
| UTY | Ubiquitously transcribed tetratricopeptide repeat | SEQ ID NOS: 14002-14014 |
| | containing, Y-linked | |
| UXS1 | UDP-glucuronate decarboxylase 1 | SEQ ID NOS: 14015-14022 |
| VASH1 | Vasohibin 1 | SEQ ID NOS: 14023-14025 |
| VCAN | Versican | SEQ ID NOS: 14026-14032 |
| VEGFA | Vascular endothelial growth factor A | SEQ ID NOS: 14033-14058 |
| VEGFB | Vascular endothelial growth factor B | SEQ ID NOS: 14059-14061 |
| VEGFC | Vascular endothelial growth factor C | SEQ ID NO: 14062 |
| FIGF | C-fos induced growth factor (vascular endothelial | SEQ ID NO: 14063 |
| | growth factor D) | |
| VGF | VGF nerve growth factor inducible | SEQ ID NOS: 14064-14066 |
| VIP | Vasoactive intestinal peptide | SEQ ID NOS: 14067-14069 |
| VIPR2 | Vasoactive intestinal peptide receptor 2 | SEQ ID NOS: 14070-14073 |
| VIT | Vitrin | SEQ ID NOS: 14074-14081 |
| VKORC1 | Vitamin K epoxide reductase complex, subunit 1 | SEQ ID NOS: 14082-14089 |
| VLDLR | Very low density lipoprotein receptor | SEQ ID NOS: 14090-14092 |
| VMO1 | Vitelline membrane outer layer 1 homolog (chicken) | SEQ ID NOS: 14093-14096 |
| VNN1 | Vanin 1 | SEQ ID NO: 14097 |
| VNN2 | Vanin 2 | SEQ ID NOS: 14098-14111 |
| VNN3 | Vanin 3 | SEQ ID NOS: 14112-14123 |
| VOPP1 | Vesicular, overexpressed in cancer, prosurvival | SEQ ID NOS: 14124-14136 |
| | protein 1 | |
| VPREB1 | Pre-B lymphocyte 1 | SEQ ID NOS: 14137-14138 |
| VPREB3 | Pre-B lymphocyte 3 | SEQ ID NOS: 14139-14140 |
| VPS37B | Vacuolar protein sorting 37 homolog B (S. cerevisiae) | SEQ ID NOS: 14141-14143 |
| VPS51 | Vacuolar protein sorting 51 homolog (S. cerevisiae) | SEQ ID NOS: 14144-14155 |
| VSIG1 | V-set and immunoglobulin domain containing 1 | SEQ ID NOS: 14156-14158 |
| VSIG10 | V-set and immunoglobulin domain containing 10 | SEQ ID NOS: 14159-14160 |
| VSTM1 | V-set and transmembrane domain containing 1 | SEQ ID NOS: 14161-14167 |
| VSTM2A | V-set and transmembrane domain containing 2A | SEQ ID NOS: 14168-14171 |
| VSTM2B | V-set and transmembrane domain containing 2B | SEQ ID NO: 14172 |
| VSTM2L | V-set and transmembrane domain containing 2 like | SEQ ID NOS: 14173-14175 |
| VSTM4 | V-set and transmembrane domain containing 4 | SEQ ID NOS: 14176-14177 |
| VTN | Vitronectin | SEQ ID NOS: 14178-14179 |
| VWA1 | Von Willebrand factor A domain containing 1 | SEQ ID NOS: 14180-14183 |
| VWA2 | Von Willebrand factor A domain containing 2 | SEQ ID NOS: 14184-14185 |
| VWA5B2 | Von Willebrand factor A domain containing 5B2 | SEQ ID NOS: 14186-14187 |
| VWA7 | Von Willebrand factor A domain containing 7 | SEQ ID NO: 14188 |
| VWC2 | Von Willebrand factor C domain containing 2 | SEQ ID NO: 14189 |
| VWC2L | Von Willebrand factor C domain containing protein | SEQ ID NOS: 14190-14191 |
| | 2-like | |
| VWCE | Von Willebrand factor C and EGF domains | SEQ ID NOS: 14192-14196 |
| VWDE | Von Willebrand factor D and EGF domains | SEQ ID NOS: 14197-14202 |
| VWF | Von Willebrand factor | SEQ ID NOS: 14203-14205 |
| WDR25 | WD repeat domain 25 | SEQ ID NOS: 14206-14212 |
| WDR81 | WD repeat domain 81 | SEQ ID NOS: 14213-14222 |
| WDR90 | WD repeat domain 90 | SEQ ID NOS: 14223-14230 |
| WFDC1 | WAP four-disulfide core domain 1 | SEQ ID NOS: 14231-14233 |
| WFDC10A | WAP four-disulfide core domain 10A | SEQ ID NO: 14234 |
| WFDC10B | WAP four-disulfide core domain 10B | SEQ ID NOS: 14235-14236 |
| WFDC11 | WAP four-disulfide core domain 11 | SEQ ID NOS: 14237-14239 |
| WFDC12 | WAP four-disulfide core domain 12 | SEQ ID NO: 14240 |
| WFDC13 | WAP four-disulfide core domain 13 | SEQ ID NO: 14241 |
| WFDC2 | WAP four-disulfide core domain 2 | SEQ ID NOS: 14242-14246 |
| WFDC3 | WAP four-disulfide core domain 3 | SEQ ID NOS: 14247-14250 |
| WFDC5 | WAP four-disulfide core domain 5 | SEQ ID NOS: 14251-14252 |
| WFDC6 | WAP four-disulfide core domain 6 | SEQ ID NOS: 14253-14254 |
| WFDC8 | WAP four-disulfide core domain 8 | SEQ ID NOS: 14255-14256 |
| WFIKKN1 | WAP, follistatin/kazal, immunoglobulin, kunitz and | SEQ ID NO: 14257 |
| | netrin domain containing 1 | |
| WFIKKN2 | WAP, follistatin/kazal, immunoglobulin, kunitz and | SEQ ID NOS: 14258-14259 |
| | netrin domain containing 2 | |
| DFNB31 | Deafness, autosomal recessive 31 | SEQ ID NOS: 14260-14263 |
| WIF1 | WNT inhibitory factor 1 | SEQ ID NOS: 14264-14266 |
| WISP1 | WNT1 inducible signaling pathway protein 1 | SEQ ID NOS: 14267-14271 |
| WISP2 | WNT1 inducible signaling pathway protein 2 | SEQ ID NOS: 14272-14274 |
| WISP3 | WNT1 inducible signaling pathway protein 3 | SEQ ID NOS: 14275-14282 |
| WNK1 | WNK lysine deficient protein kinase 1 | SEQ ID NOS: 14283-14296 |
| WNT1 | Wingless-type MMTV integration site family, | SEQ ID NOS: 14297-14298 |
| | member 1 | |
| WNT10B | Wingless-type MMTV integration site family, | SEQ ID NOS: 14299-14303 |
| | member 10B | |
| WNT11 | Wingless-type MMTV integration site family, | SEQ ID NOS: 14304-14306 |
| | member 11 | |
| WNT16 | Wingless-type MMTV integration site family, | SEQ ID NOS: 14307-14308 |
| | member 16 | |
| WNT2 | Wingless-type MMTV integration site family | SEQ ID NOS: 14309-14311 |
| | member 2 | |
| WNT3 | Wingless-type MMTV integration site family, | SEQ ID NO: 14312 |
| | member 3 | |
| WNT3A | Wingless-type MMTV integration site family, | SEQ ID NO: 14313 |
| | member 3A | |
| WNT5A | Wingless-type MMTV integration site family, | SEQ ID NOS: 14314-14317 |
| | member 5A | |
| WNT5B | Wingless-type MMTV integration site family, | SEQ ID NOS: 14318-14324 |
| | member 5B | |
| WNT6 | Wingless-type MMTV integration site family, | SEQ ID NO: 14325 |
| | member 6 | |
| WNT7A | Wingless-type MMTV integration site family, | SEQ ID NO: 14326 |
| | member 7A | |
| WNT7B | Wingless-type MMTV integration site family, | SEQ ID NOS: 14327-14331 |
| | member 7B | |
| WNT8A | Wingless-type MMTV integration site family, | SEQ ID NOS: 14332-14335 |
| | member 8A | |
| WNT8B | Wingless-type MMTV integration site family, | SEQ ID NO: 14336 |
| | member 8B | |
| WNT9A | Wingless-type MMTV integration site family, | SEQ ID NO: 14337 |
| | member 9A | |
| WNT9B | Wingless-type MMTV integration site family, | SEQ ID NOS: 14338-14340 |
| | member 9B | |
| WSB1 | WD repeat and SOCS box containing 1 | SEQ ID NOS: 14341-14350 |
| WSCD1 | WSC domain containing 1 | SEQ ID NOS: 14351-14360 |
| WSCD2 | WSC domain containing 2 | SEQ ID NOS: 14361-14364 |
| XCL1 | Chemokine (C motif) ligand 1 | SEQ ID NO: 14365 |
| XCL2 | Chemokine (C motif) ligand 2 | SEQ ID NO: 14366 |
| XPNPEP2 | X-prolyl aminopeptidase (aminopeptidase P) 2, | SEQ ID NOS: 14367-14368 |
| | membrane-bound | |
| XXYLT1 | Xyloside xylosyltransferase 1 | SEQ ID NOS: 14369-14374 |
| XYLT1 | Xylosyltransferase I | SEQ ID NO: 14375 |
| XYLT2 | Xylosyltransferase II | SEQ ID NOS: 14376-14381 |
| ZFYVE21 | Zinc finger, FYVE domain containing 21 | SEQ ID NOS: 14382-14386 |
| ZG16 | Zymogen granule protein 16 | SEQ ID NO: 14387 |
| ZG16B | Zymogen granule protein 16B | SEQ ID NOS: 14388-14391 |
| ZIC4 | Zic family member 4 | SEQ ID NOS: 14392-14400 |
| ZNF207 | Zinc finger protein 207 | SEQ ID NOS: 14401-14411 |
| ZNF26 | Zinc finger protein 26 | SEQ ID NOS: 14412-14415 |
| ZNF34 | Zinc finger protein 34 | SEQ ID NOS: 14416-14419 |
| ZNF419 | Zinc finger protein 419 | SEQ ID NOS: 14420-14434 |
| ZNF433 | Zinc finger protein 433 | SEQ ID NOS: 14435-14444 |
| ZNF449 | Zinc finger protein 449 | SEQ ID NOS: 14445-14446 |
| ZNF488 | Zinc finger protein 488 | SEQ ID NOS: 14447-14448 |
| ZNF511 | Zinc finger protein 511 | SEQ ID NOS: 14449-14450 |
| ZNF570 | Zinc finger protein 570 | SEQ ID NOS: 14451-14456 |
| ZNF691 | Zinc finger protein 691 | SEQ ID NOS: 14457-14464 |
| ZNF98 | Zinc finger protein 98 | SEQ ID NOS: 14465-14468 |
| ZPBP | Zona pellucida binding protein | SEQ ID NOS: 14469-14472 |
| ZPBP2 | Zona pellucida binding protein 2 | SEQ ID NOS: 14473-14476 |
| ZSCAN29 | Zinc finger and SCAN domain containing 29 | SEQ ID NOS: 14477-14483 |

In certain embodiments, the therapeutic protein is not secreted, but rather functions intracellularly.

In certain embodiments, the therapeutic protein is not secreted, but rather directs a modified cell of the disclosure to a cell niche of a subject's body.

In certain embodiments of the methods of the disclosure, the subject has a disease or disorder and the plurality of therapeutic immune cells or immune precursor cells improves a sign or symptom of the disease or disorder, optionally by providing a therapeutic protein systemically or locally within the subject that acts upon the immune cell, the immune precursor cell or a second cell in the subject. Exemplary therapeutic secreted proteins may be used as a monotherapy or in combination with another therapy in the treatment or prevention of any disease or disorder. These secreted proteins may be used as a monotherapy or in combination with another therapy for enzyme replacement and/or administration of biologic therapeutics.

### Inducible Proapoptotic Polypeptides

Inducible proapoptotic polypeptides of the disclosure are superior to existing inducible polypeptides because the inducible proapoptotic polypeptides of the disclosure are far less immunogenic. While inducible proapoptotic polypeptides of the disclosure are recombinant polypeptides, and, therefore, non-naturally occurring, the sequences that are recombined to produce the inducible proapoptotic polypeptides of the disclosure do not comprise non-human sequences that the host human immune system could recognize as “non-self” and, consequently, induce an immune response in the subject receiving an inducible proapoptotic polypeptide of the disclosure, a cell comprising the inducible proapoptotic polypeptide or a composition comprising the inducible proapoptotic polypeptide or the cell comprising the inducible proapoptotic polypeptide.

Modified cells and/or transposons of the disclosure may comprise an inducible proapoptotic polypeptide comprising (a) a ligand binding region, (b) a linker, and (c) a proapoptotic polypeptide, wherein the inducible proapoptotic polypeptide does not comprise a non-human sequence. In certain embodiments, the non-human sequence comprises a restriction site. In certain embodiments, the ligand binding region may be a multimeric ligand binding region. Inducible proapoptotic polypeptides of the disclosure may also be referred to as an “iC9 safety switch”. In certain embodiments, modified cells and/or transposons of the disclosure may comprise an inducible caspase polypeptide comprising (a) a ligand binding region, (b) a linker, and (c) a caspase polypeptide, wherein the inducible proapoptotic polypeptide does not comprise a non-human sequence. In certain embodiments, modified cells and/or transposons of the disclosure may comprise an inducible caspase polypeptide comprising (a) a ligand binding region, (b) a linker, and (c) a caspase polypeptide, wherein the inducible proapoptotic polypeptide does not comprise a non-human sequence. In certain embodiments, transposons of the disclosure may comprise an inducible caspase polypeptide comprising (a) a ligand binding region, (b) a linker, and (c) a truncated caspase 9 polypeptide, wherein the inducible proapoptotic polypeptide does not comprise a non-human sequence. In certain embodiments of the inducible proapoptotic polypeptides, inducible caspase polypeptides or truncated caspase 9 polypeptides of the disclosure, the ligand binding region may comprise a FK506 binding protein 12 (FKBP12) polypeptide. In certain embodiments, the amino acid sequence of the ligand binding region that comprise a FK506 binding protein 12 (FKBP12) polypeptide may comprise a modification at position 36 of the sequence. The modification may be a substitution of valine (V) for phenylalanine (F) at position 36 (F36V).

In certain embodiments, the FKBP12 polypeptide is encoded by an amino acid sequence comprising

(SEQ ID NO: 14635)
GVQVETISPGDGRTFPKRGQTCVVHYTGMLEDGKKVDSSRDRNKPFKF
MLGKQEVIRGWEEGVAQMSVGQRAKLTISPDVAYGATGHPGIIPPHAT
LVFDVELLKLE.

In certain embodiments, the FKBP12 polypeptide is encoded by a nucleic acid sequence comprising

(SEQ ID NO: 14636)
GGGGTCCAGGTCGAGACTATTTCACCAGGGGATGGGCGAACATTTCCA
AAAAGGGGCCAGACTTGCGTCGTGCATTACACCGGGATGCTGGAGGAC
GGGAAGAAAGTGGACAGCTCCAGGGATCGCAACAAGCCCTTCAAGTTC
ATGCTGGGAAAGCAGGAAGTGATCCGAGGATGGGAGGAAGGCGTGGCA
CAGATGTCAGTCGGCCAGCGGGCCAAACTGACCATTAGCCCTGACTAC
GCTTATGGAGCAACAGGCCACCCAGGGATCATTCCCCCTCATGCCACC
CTGGTCTTCGATGTGGAACTGCTGAAGCTGGAG.

In certain embodiments, the induction agent specific for the ligand binding region may comprise a FK506 binding protein 12 (FKBP12) polypeptide having a substitution of valine (V) for phenylalanine (F) at position 36 (F36V) comprises AP20187 and/or AP1903, both synthetic drugs.

In certain embodiments of the inducible proapoptotic polypeptides, inducible caspase polypeptides or truncated caspase 9 polypeptides of the disclosure, the linker region is encoded by an amino acid comprising GGGGS (SEQ ID NO: 14637) or a nucleic acid sequence comprising GGAGGAGGAGGATCC (SEQ ID NO: 14638). In certain embodiments, the nucleic acid sequence encoding the linker does not comprise a restriction site.

In certain embodiments of the truncated caspase 9 polypeptides of the disclosure, the truncated caspase 9 polypeptide is encoded by an amino acid sequence that does not comprise an arginine (R) at position 87 of the sequence. Alternatively, or in addition, in certain embodiments of the inducible proapoptotic polypeptides, inducible caspase polypeptides or truncated caspase 9 polypeptides of the disclosure, the truncated caspase 9 polypeptide is encoded by an amino acid sequence that does not comprise an alanine (A) at position 282 the sequence. In certain embodiments of the inducible proapoptotic polypeptides, inducible caspase polypeptides or truncated caspase 9 polypeptides of the disclosure, the truncated caspase 9 polypeptide is encoded by an amino acid comprising

(SEQ ID NO: 14639)
GFGDVGALESLRGNADLAYILSMEPCGHCLIINNVNFCRESGLRTRTG
SNIDCEKLRRRFSSLHFMVEVKGDLTAKKMVLALLELAQQDHGALDCC
VVVILSHGCQASHLQFPGAVYGTDGCPVSVEKIVNIFNGTSCPSLGGK
PKLFFIQACGGEQKDHGFEVASTSPEDESPGSNPEPDATPFQEGLRTF
DQLDAISSLPTPSDIFVSYSTFPGFVSWRDPKSGSWYVETLDDIFEQW
AHSEDLQSLLLRVANAVSVKGIYKQMPGCFNFLRKKLFFKTS
or a nucleic acid sequence comprising
(SEQ ID NO: 14640)
TTTGGGGACGTGGGGGCCCTGGAGTCTCTGCGAGGAAATGCCGATCTG
GCTTACATCCTGAGCATGGAACCCTGCGGCCACTGTCTGATCATTAAC
AATGTGAACTTCTGCAGAGAAAGCGGACTGCGAACACGGACTGGCTCC
AATATTGACTGTGAGAAGCTGCGGAGAAGGTTCTCTAGTCTGCACTTT
ATGGTCGAAGTGAAAGGGGATCTGACCGCCAAGAAAATGGTGCTGGCC
CTGCTGGAGCTGGCTCAGCAGGACCATGGAGCTCTGGATTGCTGCGTG
GTCGTGATCCTGTCCCACGGGTGCCAGGCTTCTCATCTGCAGTTCCCC
GGAGCAGTGTACGGAACAGACGGCTGTCCTGTCAGCGTGGAGAAGATC
GTCAACATCTTCAACGGCACTTCTTGCCCTAGTCTGGGGGGAAAGCCA
AAACTGTTCTTTATCCAGGCCTGTGGCGGGGAACAGAAAGATCACGGC
TTCGAGGTGGCCAGCACCAGCCCTGAGGACGAATCACCAGGGAGCAAC
CCTGAACCAGATGCAACTCCATTCCAGGAGGGACTGAGGACCTTTGAC
CAGCTGGATGCTATCTCAAGCCTGCCCACTCCTAGTGACATTTTCGTG
TCTTACAGTACCTTCCCAGGCTTTGTCTCATGGCGCGATCCCAAGTCA
GGGAGCTGGTACGTGGAGACACTGGACGACATCTTTGAACAGTGGGCC
CATTCAGAGGACCTGCAGAGCCTGCTGCTGCGAGTGGCAAACGCTGTC
TCTGTGAAGGGCATCTACAAACAGATGCCCGGGTGCTTCAATTTTCTG
AGAAAGAAACTGTTCTTTAAGACTTCC.

In certain embodiments of the inducible proapoptotic polypeptides, wherein the polypeptide comprises a truncated caspase 9 polypeptide, the inducible proapoptotic polypeptide is encoded by an amino acid sequence comprising

(SEQ ID NO: 14641)
GVQVETISPGDGRTFPKRGQTCVVHYTGMLEDGKKVDSSRDRNKPFKF
MLGKQEVIRGWEEGVAQMSVGQRAKLTISPDVAYGATGHPGIIPPHAT
LVFDVELLKLEGGGGSGFGDVGALESLRGNADLAYILSMEPCGHCLII
NNVNFCRESGLRTRTGSNIDCEKLRRRFSSLHFMVEVKGDLTAKKMVL
ALLELAQQDHGALDCCVVVILSHGCQASHLQFPGAVYGTDGCPVSVEK
IVNIFNGTSCPSLGGKPKLFFIQACGGEQKDHGFEVASTSPEDESPGS
NPEPDATPFQEGLRTFDQLDAIS SLPTP SDIFVSYSTFPGFVSWRD
PKSGSWYVETLDDIFEQWAHSEDLQSLLLRVANAVSVKGIYKQMPGCF
NFLRKKLFFKTS
or the nucleic acid sequence comprising
(SEQ ID NO: 14642)
ggggtccaggtcgagactatttcaccaggggatgggcgaacatttcca
aaaaggggccagacttgcgtcgtgcattacaccgggatgctggaggac
gggaagaaagtggacagctccagggatcgcaacaagcccttcaagttc
atgctgggaaagcaggaagtgatccgaggatgggaggaaggcgtggca
cagatgtcagtcggccagcgggccaaactgaccattagccctgactac
gcttatggagcaacaggccacccagggatcattccccctcatgccacc
ctggtcttcgatgtggaactgctgaagctggagggaggaggaggatcc
ggatttggggacgtgggggccctggagtctctgcgaggaaatgccgat
ctggcttacatcctgagcatggaaccctgcggccactgtctgatcatt
aacaatgtgaacttctgcagagaaagcggactgcgaacacggactggc
tccaatattgactgtgagaagctgcggagaaggttctctagtctgcac
tttatggtcgaagtgaaaggggatctgaccgccaagaaaatggtgctg
gccctgctggagctggctcagcaggaccatggagctctggattgctgc
gtggtcgtgatcctgtcccacgggtgccaggcttctcatctgcagttc
cccggagcagtgtacggaacagacggctgtcctgtcagcgtggagaag
atcgtcaacatcttcaacggcacttcttgccctagtctggggggaaag
ccaaaactgttctttatccaggcctgtggcggggaacagaaagatcac
ggcttcgaggtggccagcaccagccctgaggacgaatcaccagggagc
aaccctgaaccagatgcaactccattccaggagggactgaggaccttt
gaccagctggatgctatctcaagcctgcccactcctagtgacattttc
gtgtcttacagtaccttcccaggctttgtctcatggcgcgatcccaag
tcagggagctggtacgtggagacactggacgacatctttgaacagtgg
gcccattcagaggacctgcagagcctgctgctgcgagtggcaaacgct
gtctctgtgaagggcatctacaaacagatgcccgggtgcttcaattac
tgagaaagaaactgttctttaagacttcc.

### Construct Elements

Transposons and other delivery vectors of the disclosure may comprise at least one self-cleaving peptide(s) located, for example, between one or more of a sequence encoding an inducible proapoptotic polypeptide of the disclosure, a sequence encoding a therapeutic protein of the disclosure and a selection gene of the disclosure.

Transposons and other delivery vectorsof the disclosure may comprise at least two self-cleaving peptide(s), a first self-cleaving peptide located, for example, upstream or immediately upstream of an inducible proapoptotic polypeptide of the disclosure of the disclosure and a second first self-cleaving peptide located, for example, downstream or immediately upstream of an inducible proapoptotic polypeptide of the disclosure of the disclosure.

The at least one self-cleaving peptide may comprise, for example, a T2A peptide, GSG-T2A peptide, an E2A peptide, a GSG-E2A peptide, an F2A peptide, a GSG-F2A peptide, a P2A peptide, or a GSG-P2A peptide. A T2A peptide may comprise an amino acid sequence comprising EGRGSLLTCGDVEENPGP (SEQ ID NO: 14643) or a sequence having at least 70%, 80%, 90%, 95%, or 99% identity to the amino acid sequence comprising EGRGSLLTCGDVEENPGP (SEQ ID NO: 14643). A GSG-T2A peptide may comprise an amino acid sequence comprising GSGEGRGSLLTCGDVEENPGP (SEQ ID NO: 14644) or a sequence having at least 70%, 80%, 90%, 95%, or 99% identity to the amino acid sequence comprising GSGEGRGSLLTCGDVEENPGP (SEQ ID NO: 14644). A GSG-T2A peptide may comprise a nucleic acid sequence comprising

(SEQ ID NO: 14645)
ggatctggagagggaaggggaagcctgctgacctgtggagacgtggagg
aaaacccaggacca.

An E2A peptide may comprise an amino acid sequence comprising QCTNYALLKLAGDVESNPGP (SEQ ID NO: 14646) or a sequence having at least 70%, 80%, 90%, 95%, or 99% identity to the amino acid sequence comprising QCTNYALLKLAGDVESNPGP (SEQ ID NO: 14646). A GSG-E2A peptide may comprise an amino acid sequence comprising GSGQCTNYALLKLAGDVESNPGP (SEQ ID NO: 14647) or a sequence having at least 70%, 80%, 90%, 95%, or 99% identity to the amino acid sequence comprising GSGQCTNYALLKLAGDVESNPGP (SEQ ID NO: 14647). An F2A peptide may comprise an amino acid sequence comprising VKQTLNFDLLKLAGDVESNPGP (SEQ ID NO: 14648) or a sequence having at least 70%, 80%, 90%, 95%, or 99% identity to the amino acid sequence comprising VKQTLNFDLLKLAGDVESNPGP (SEQ ID NO: 14648). A GSG-F2A peptide may comprise an amino acid sequence comprising GSGVKQTLNFDLLKLAGDVESNPGP (SEQ ID NO: 14649) or a sequence having at least 70%, 80%, 90%, 95%, or 99% identity to the amino acid sequence comprising GSGVKQTLNFDLLKLAGDVESNPGP (SEQ ID NO: 14649). A P2A peptide may comprise an amino acid sequence comprising ATNFSLLKQAGDVEENPGP (SEQ ID NO: 14650) or a sequence having at least 70%, 80%, 90%, 95%, or 99% identity to the amino acid sequence comprising ATNFSLLKQAGDVEENPGP (SEQ ID NO: 14650). A GSG-P2A peptide may comprise an amino acid sequence comprising GSGATNFSLLKQAGDVEENPGP (SEQ ID NO: 14651) or a sequence having at least 70%, 80%, 90%, 95%, or 99% identity to the amino acid sequence comprising GSGATNFSLLKQAGDVEENPGP (SEQ ID NO: 14651).

Transposons and other delivery vectors of the disclosure may comprise a first and a second self-cleaving peptide, the first self-cleaving peptide located, for example, upstream of one or more of a sequence encoding a therapeutic protein of the disclosure the second self-cleaving peptide located, for example, downstream of a sequence encoding a therapeutic protein of the disclosure. The first and/or the second self-cleaving peptide may comprise, for example, a T2A peptide, GSG-T2A peptide, an E2A peptide, a GSG-E2A peptide, an F2A peptide, a GSG-F2A peptide, a P2A peptide, or a GSG-P2A peptide. A T2A peptide may comprise an amino acid sequence comprising EGRGSLLTCGDVEENPGP (SEQ ID NO: 14643) or a sequence having at least 70%, 80%, 90%, 95%, or 99% identity to the amino acid sequence comprising EGRGSLLTCGDVEENPGP (SEQ ID NO: 14643). A GSG-T2A peptide may comprise an amino acid sequence comprising GSGEGRGSLLTCGDVEENPGP (SEQ ID NO: 14644) or a sequence having at least 70%, 80%, 90%, 95%, or 99% identity to the amino acid sequence comprising GSGEGRGSLLTCGDVEENPGP (SEQ ID NO: 14644). A GSG-T2A peptide may comprise a nucleic acid sequence comprising

(SEQ ID NO: 14645)
ggatctggagagggaaggggaagcctgctgacctgtggagacgtggagg
aaaacccaggacca.

An E2A peptide may comprise an amino acid sequence comprising QCTNYALLKLAGDVESNPGP (SEQ ID NO: 14646) or a sequence having at least 70%, 80%, 90%, 95%, or 99% identity to the amino acid sequence comprising QCTNYALLKLAGDVESNPGP (SEQ ID NO: 14646). A GSG-E2A peptide may comprise an amino acid sequence comprising GSGQCTNYALLKLAGDVESNPGP (SEQ ID NO: 14647) or a sequence having at least 70%, 80%, 90%, 95%, or 99% identity to the amino acid sequence comprising GSGQCTNYALLKLAGDVESNPGP (SEQ ID NO: 14647). An F2A peptide may comprise an amino acid sequence comprising VKQTLNFDLLKLAGDVESNPGP (SEQ ID NO: 14648) or a sequence having at least 70%, 80%, 90%, 95%, or 99% identity to the amino acid sequence comprising VKQTLNFDLLKLAGDVESNPGP (SEQ ID NO: 14648). A GSG-F2A peptide may comprise an amino acid sequence comprising GSGVKQTLNFDLLKLAGDVESNPGP (SEQ ID NO: 14649) or a sequence having at least 70%, 80%, 90%, 95%, or 99% identity to the amino acid sequence comprising GSGVKQTLNFDLLKLAGDVESNPGP (SEQ ID NO: 14649). A P2A peptide may comprise an amino acid sequence comprising ATNFSLLKQAGDVEENPGP (SEQ ID NO: 14650) or a sequence having at least 70%, 80%, 90%, 95%, or 99% identity to the amino acid sequence comprising ATNFSLLKQAGDVEENPGP (SEQ ID NO: 14650). A GSG-P2A peptide may comprise an amino acid sequence comprising GSGATNFSLLKQAGDVEENPGP (SEQ ID NO: 14651) or a sequence having at least 70%, 80%, 90%, 95%, or 99% identity to the amino acid sequence comprising GSGATNFSLLKQAGDVEENPGP (SEQ ID NO: 14651).

Transposons of the disclosure may comprise a selection gene. The selection gene may encode a gene product essential for cell viability and survival. The selection gene may encode a gene product essential for cell viability and survival when challenged by selective cell culture conditions. Selective cell culture conditions may comprise a compound harmful to cell viability or survival and wherein the gene product confers resistance to the compound.

By “stable transformation” is intended that the polynucleotide construct introduced into a cell integrates into the genome of the host and is capable of being inherited by progeny thereof.

By “transient transformation” is intended that a polynucleotide construct introduced into the host does not integrate into the genome of the host.

All percentages and ratios are calculated based on the total composition unless otherwise indicated.

Every maximum numerical limitation given throughout this disclosure includes every lower numerical limitation, as if such lower numerical limitations were expressly written herein. Every minimum numerical limitation given throughout this disclosure will include every higher numerical limitation, as if such higher numerical limitations were expressly written herein. Every numerical range given throughout this disclosure will include every narrower numerical range that falls within such broader numerical range, as if such narrower numerical ranges were all expressly written herein.

The values disclosed herein are not to be understood as being strictly limited to the exact numerical values recited. Instead, unless otherwise specified, each such value is intended to mean both the recited value and a functionally equivalent range surrounding that value. For example, a value disclosed as “20 μm” is intended to mean “about 20 μm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the disclosure have been illustrated and described, various other changes and modifications can be made without departing from the spirit and scope of the disclosure. The scope of the appended claims includes all such changes and modifications that are within the scope of this disclosure.

### EXAMPLES

In order that the invention disclosed herein may be more efficiently understood, examples are provided below. It should be understood that these examples are for illustrative purposes only and are not to be construed as limiting the invention in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### Example 1: Ex Vivo Genetic Modification of T Cells

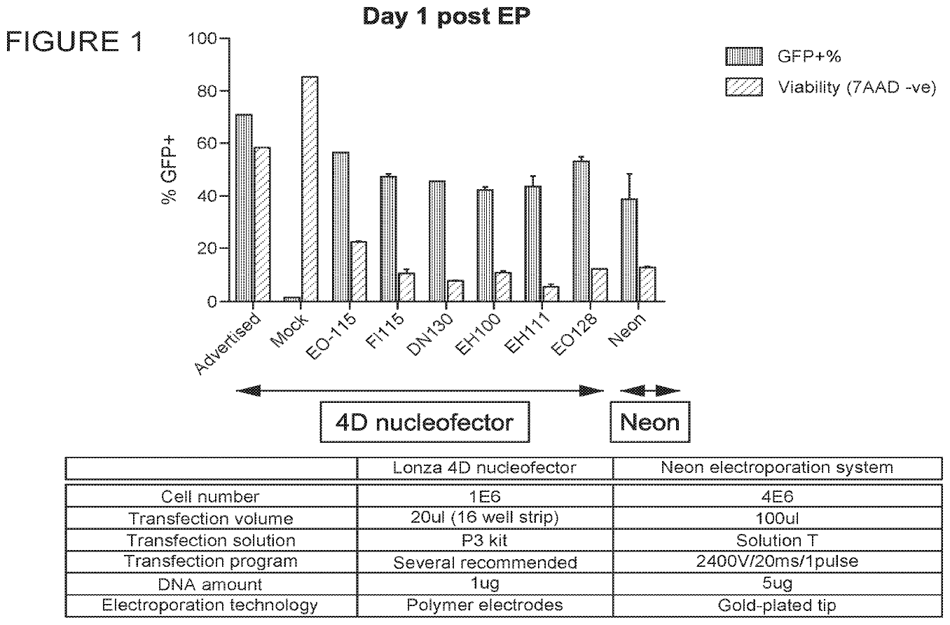

The piggyBac™ (PB) transposon system was used for genetically modifying human lymphocytes for production of autologous CAR-T immunotherapies and other applications. T Lymphocytes purified from patient blood or apheresis product was electroporated with a plasmid DNA transposon and a transposase. Several different electroporation systems have been used for T cell delivery of the transposon system, including the Neon (Thermo Fisher), BTX ECM 830 (Harvard Apparatus), Gene Pulser (BioRad), MaxCyte PulseAgile (MaxCyte), and the Amaxa 2B and Amaxa 4D (Lonza). Some were tested using manufacturer provided or recommended electroporation buffer, as well as several in-house developed buffers. Results were consistent with the prevailing dogma that resting T lymphocytes are particularly refractory to DNA transfection and that there appeared to be an inverse relationship between electroporation efficiency, as measured by GFP expression from the electroporated plasmid, and cell viability. FIG. 1 shows an example of an experiment testing multiple electroporation systems and nucleofection programs.

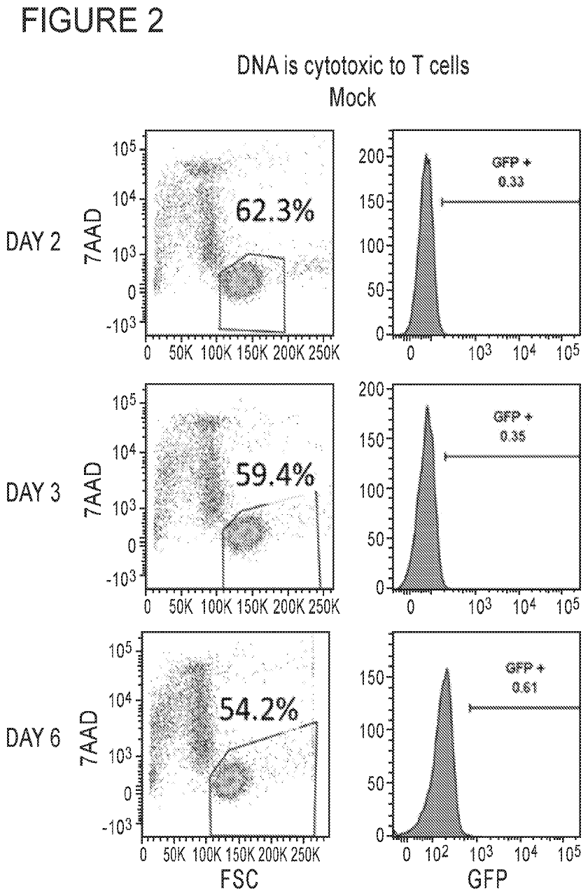

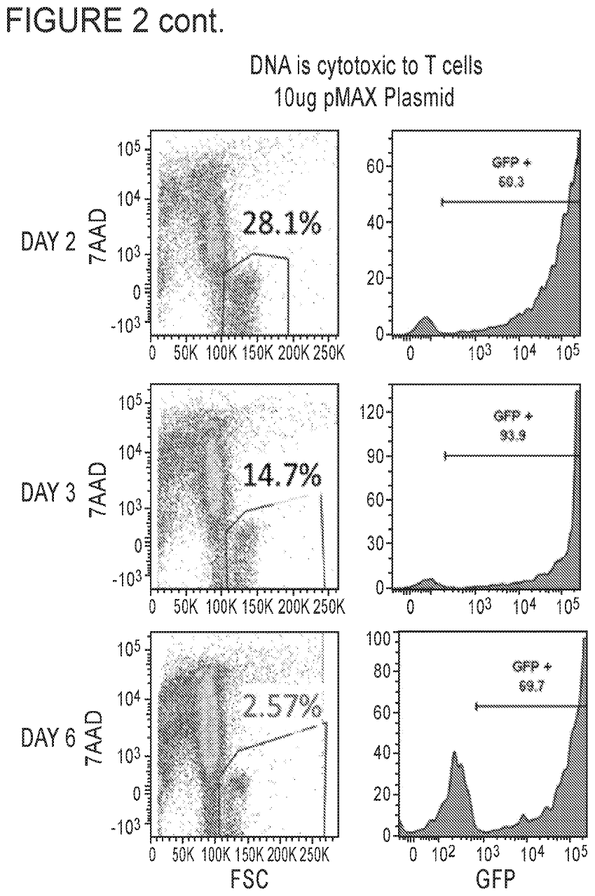

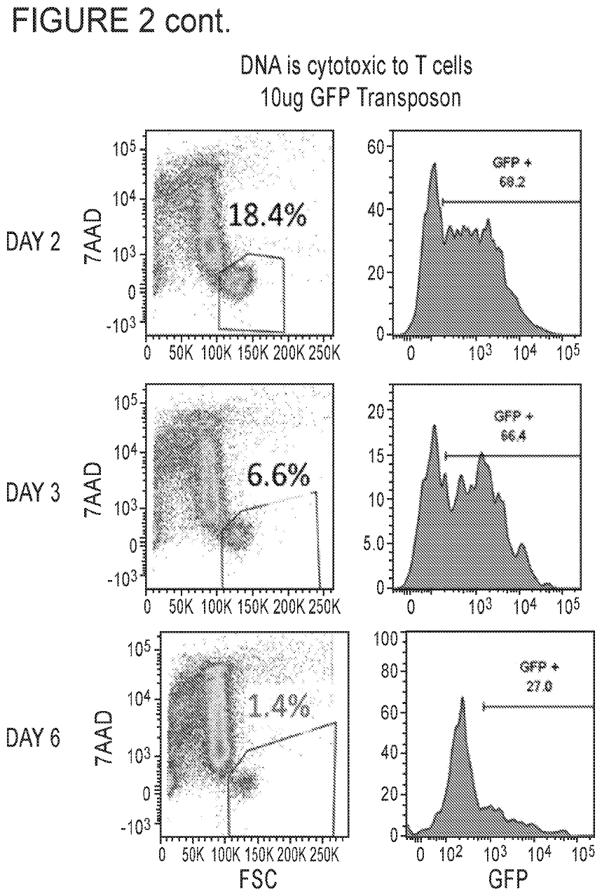

To further test whether or not plasmid DNA was toxic to T cells during nucleofection, primary human T lymphocytes were electroporated with two different DNA plasmids. The first plasmid was a pmaxGFP™ plasmid that is provided as a control plasmid in the Lonza Amaxa nucleofection kit. It is highly purified by HPLC and does not contain endotoxin at detectable levels. The second plasmid was our in-house produced PB transposon encoding a human EF1 alpha promoter driving GFP. Transfection efficiency, as measured by GFP expression from the electroporated plasmid, and cell viability was assessed by FACS at days 2, 3, and 6 post-electroporation. Data are displayed in FIG. 2. While mock electroporated cells (no plasmid DNA) exhibited relatively high levels of cell viability by day 6 post-electroporation, 54%, T cells electroporated with either plasmid were only 1.4-2.6% viable. These data show that plasmid DNA was cytotoxic to T lymphocytes. In addition, these data show that DNA-mediated toxicity was not due to transposon element such as the ITR regions or the core insulators since the pmaxGFP™ plasmid are devoid of these elements and was also cytotoxic at the same DNA concentration. Both plasmids are approximately the same size, meaning that similar amounts of DNA were electroporated into the T cells.

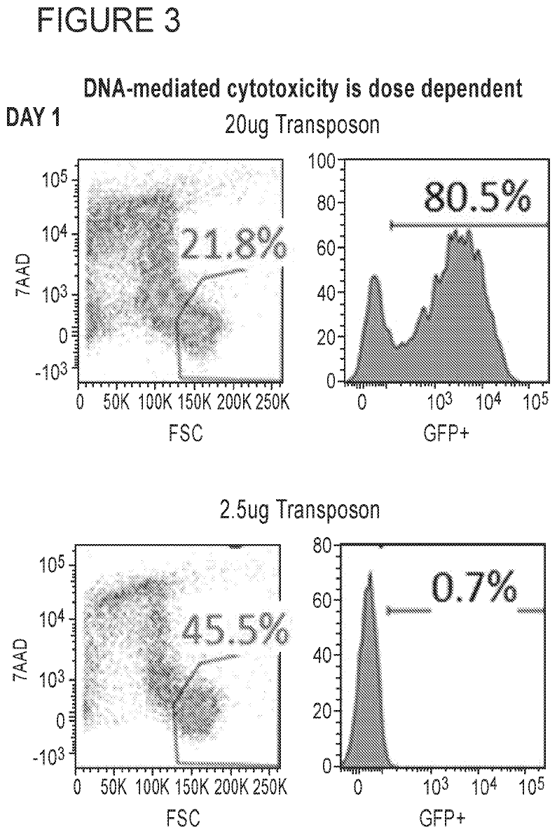

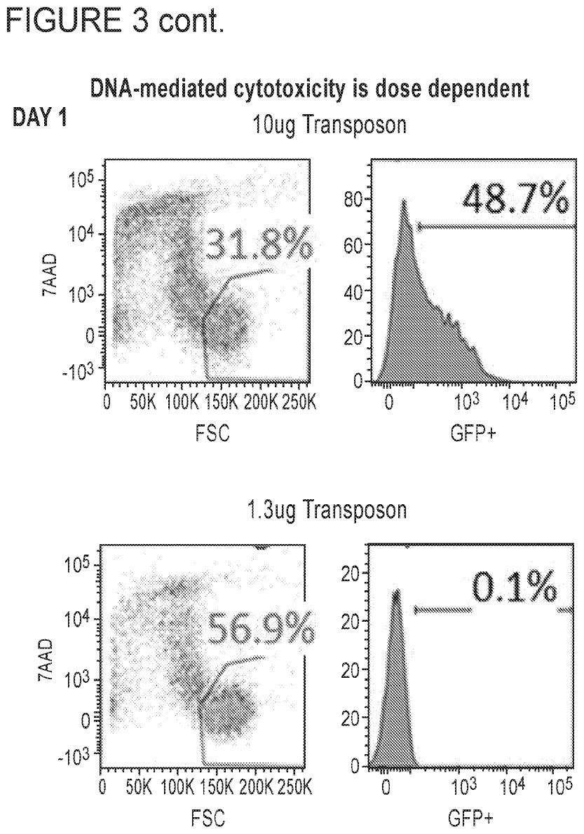

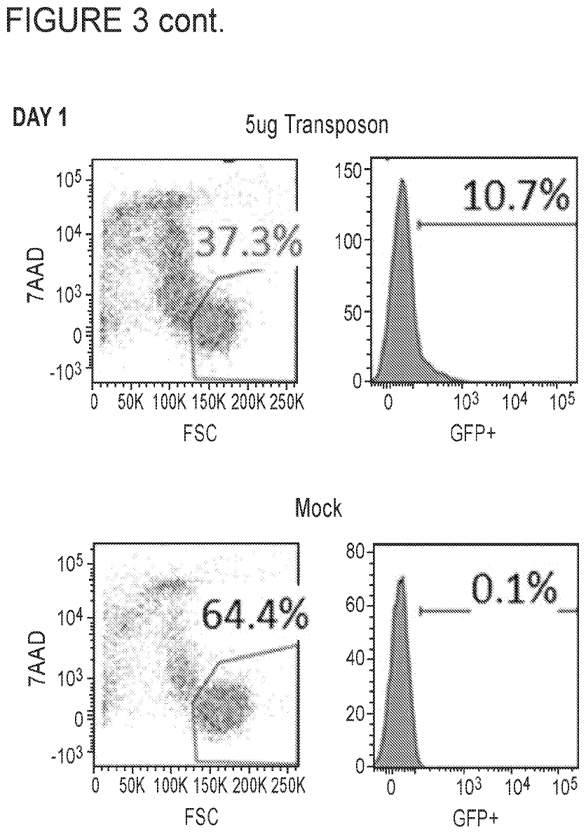

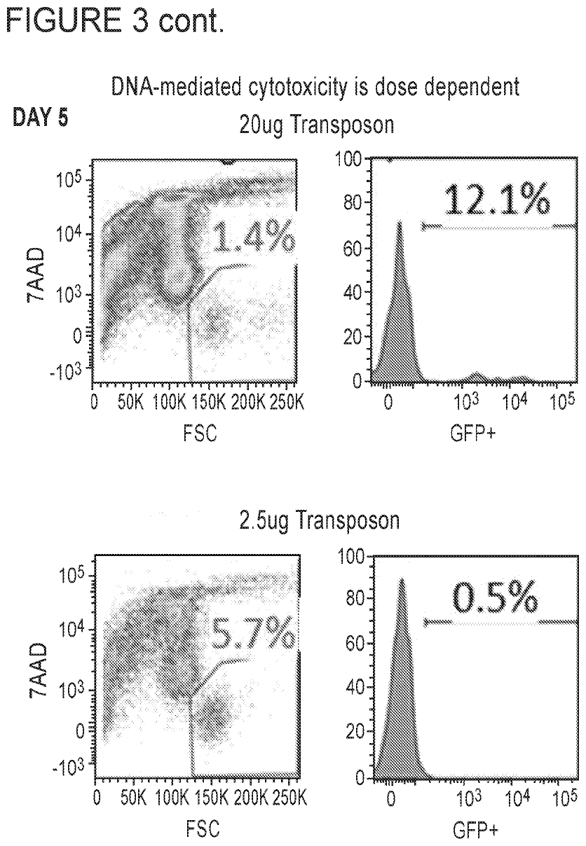

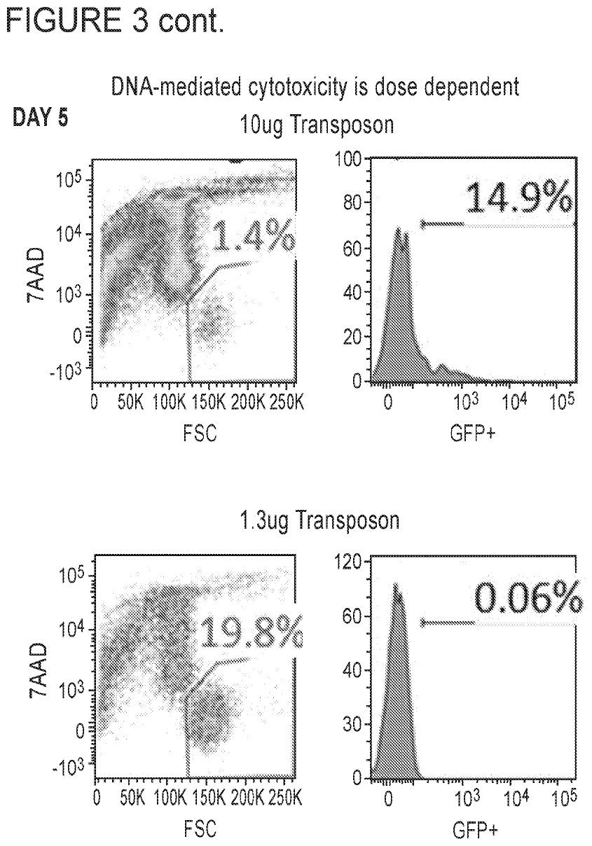

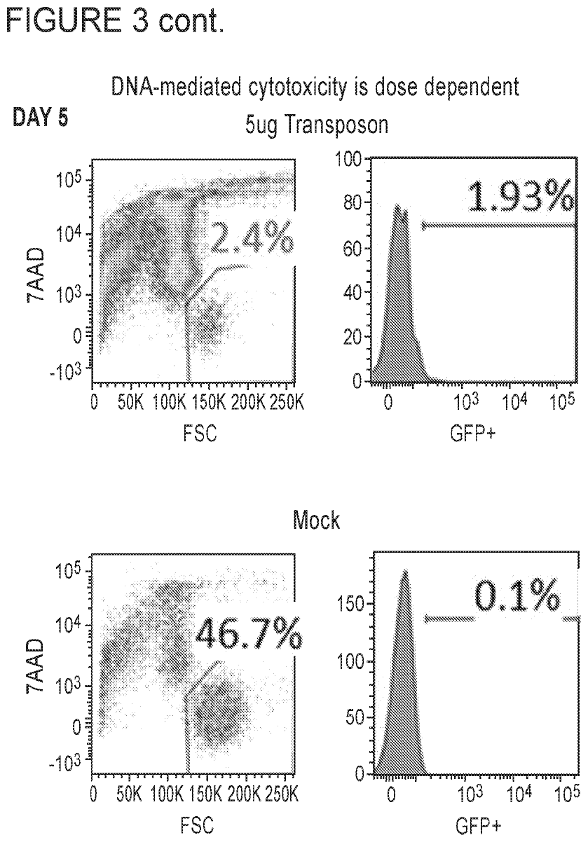

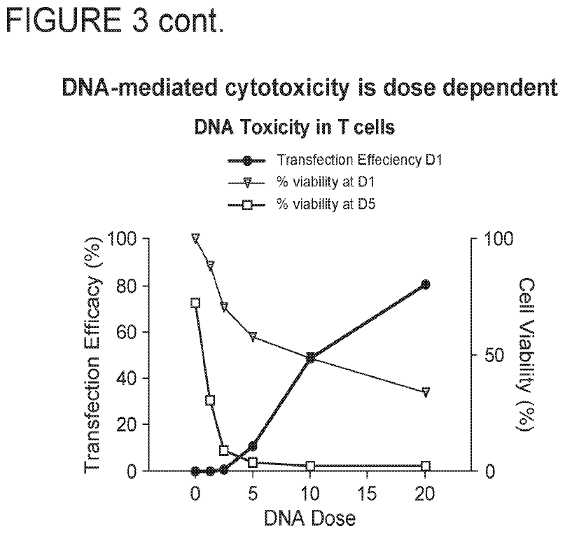

To test whether or not DNA-mediated toxicity in T cells was dose dependent, we performed a titration of our PB-GFP plasmid. FIG. 3 shows that as the dose of plasmid DNA added to the nucleofection reaction was increased incrementally (1.3, 2.5, 5.0, 10.0, and 20.0 μg of plasmid DNA), cell viability decreased as measured at both day 1 and 5 post-nucleofection. Even 1.3 μg of plasmid DNA was responsible for a 2.4-fold decrease in T cell viability by day 4.

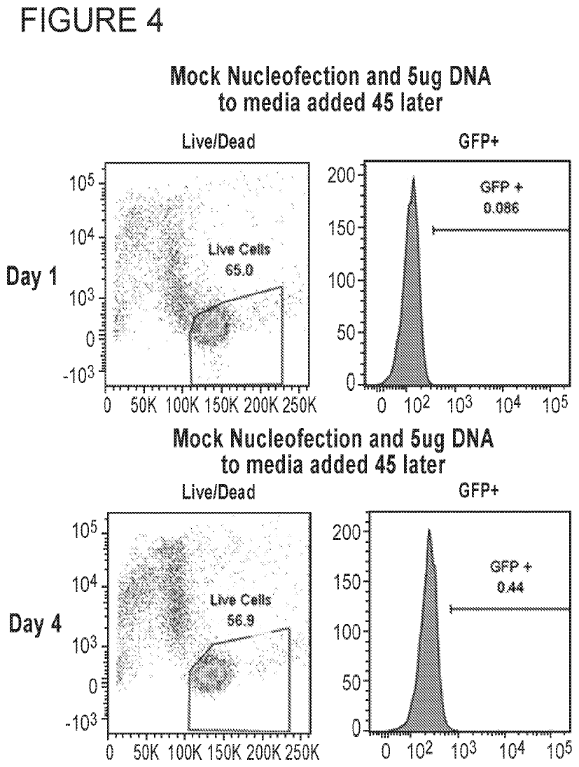

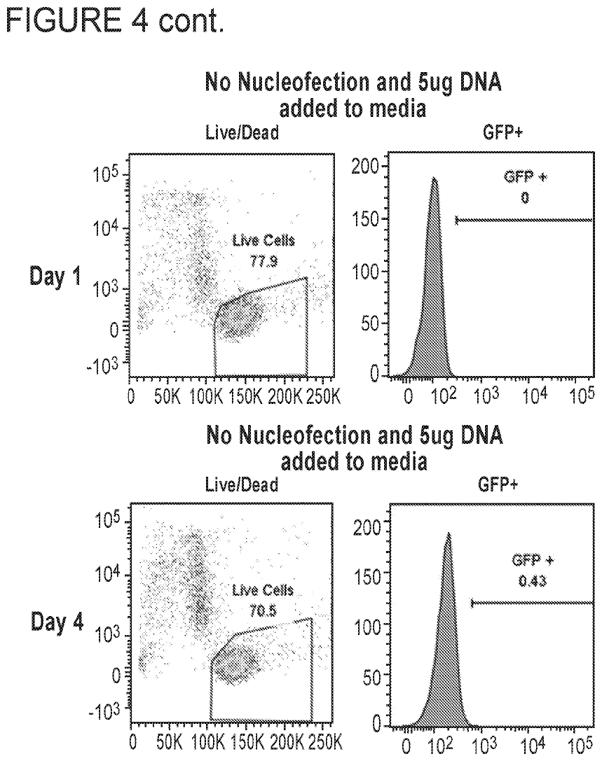

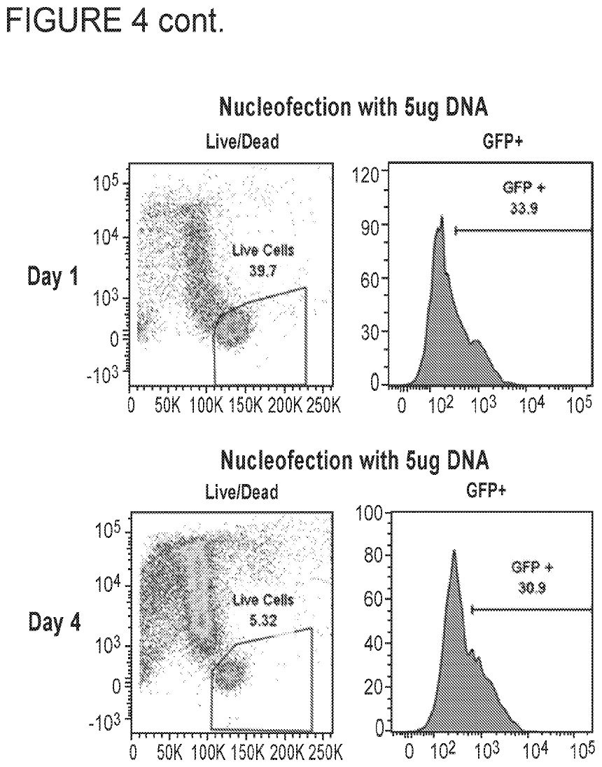

Since it was clear that plasmid DNA is toxic to T cells during nucleofection, we considered whether or not extracellular plasmid DNA was contributing to cell death. FIG. 4 shows that extracellular plasmid DNA was not cytotoxic to T cells. In that experiment, 5 μg of plasmid DNA was added to the cells 45 min post-electroporation and little cell death was observed at day 1 or day 4. Similarly, when 5 μg of plasmid DNA was added to the nucleofection reaction in the absence of electroporation, little cell death was observed. However, when the plasmid DNA was added before the electroporation reaction, the cells exhibited a 2.0-fold reduction in cell viability at day 1 and a 13.2-fold reduction at day 4.

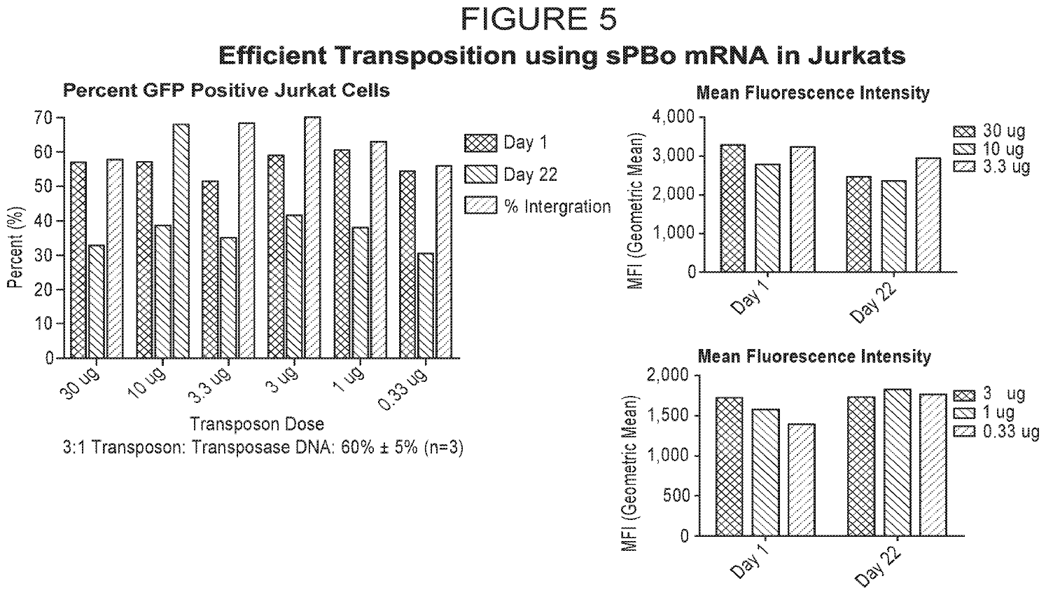

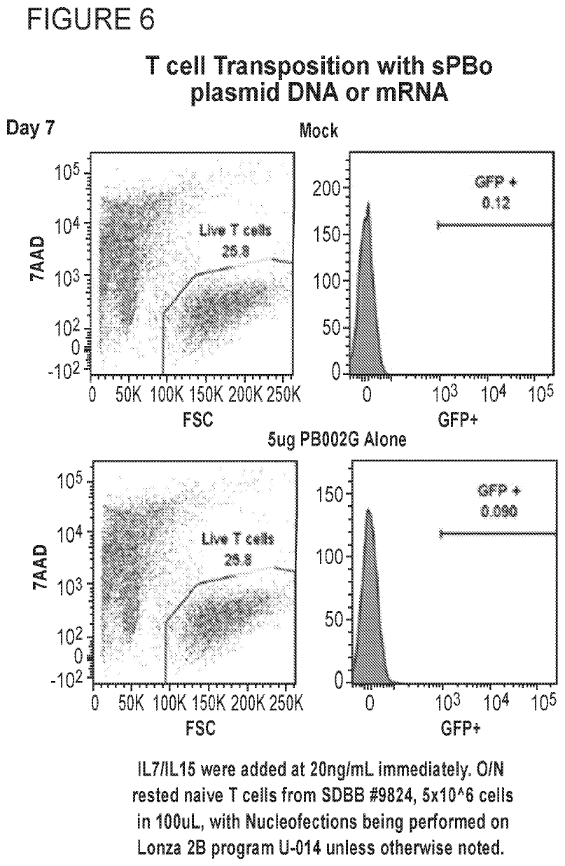

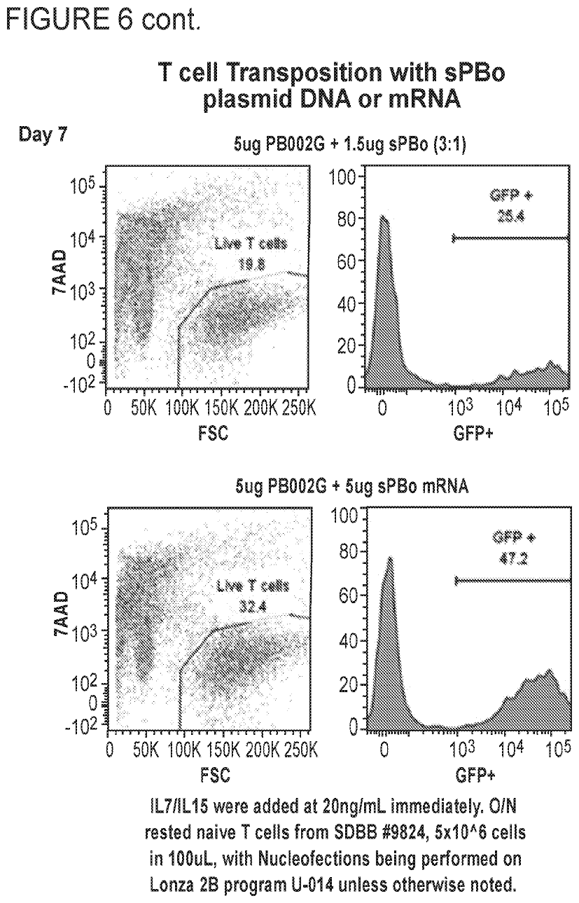

Since DNA-mediated toxicity is dose dependent, we next focused our attention on ways to reduce the total amount of DNA delivered to the T cells that is required for transposition. One relatively straightforward way of achieving this would be to deliver the transposase as encoded in mRNA instead of encoded in DNA. mRNA delivery to primary human T cells is very efficient, resulting in high transfection efficiency and high viability. We subcloned the Super piggyBac™ (SPB) transposase enzyme into our in-house mRNA production vector and produced high quality SPB mRNA. Co-delivery of PB-GFP transposon with various doses of SPB mRNA (30, 10, 3.3, 3, 1, 0.33 μg mRNA) in Jurkat cells demonstrated strong transposition at all doses tested (FIG. 5). These data show that SPB transposase can be delivered and are equally effective as either plasmid DNA or mRNA. In addition, that the amount of SPB mRNA makes little difference in overall transposition efficiency in Jurkats, in either overall percentage of GFP+ cells or in the MFI of GFP expression. To see if this also holds true for T lymphocytes, we delivered PB-GFP with either SPB plasmid DNA, at a 3:1 ratio, or 5 μg of SPB mRNA. Seven (7) days following the nucleofection reaction and the addition of IL7 and IL15, GFP transposition was assessed. FIG. 6 shows that SPB mRNA efficiently mediated transposition of the GFP transposon into T lymphocytes. Importantly, T cell viability was improved when co-delivering the SPB as an mRNA as opposed to a pDNA; 32.4% versus 25.4%, respectively. These data suggest that co-delivery of SPB as mRNA would be dose-sparing in the total amount of plasmid DNA being delivered to T cells and is thus less cytotoxic.

Since the current plasmid transposon also contains a backbone required for plasmid amplification in bacteria, it is possible to significantly reduce the total amount of DNA by excluding this sequence. This may be achieved by restriction digest of the plasmid transposon prior to the nucleofection reaction. In addition, this could be achieved by administering the transposon as a PCR product or as a Doggybone™ DNA, which is a double stranded DNA that is produced in vitro by a mechanism that excludes the initial backbone elements required for bacterial replication of the plasmid.

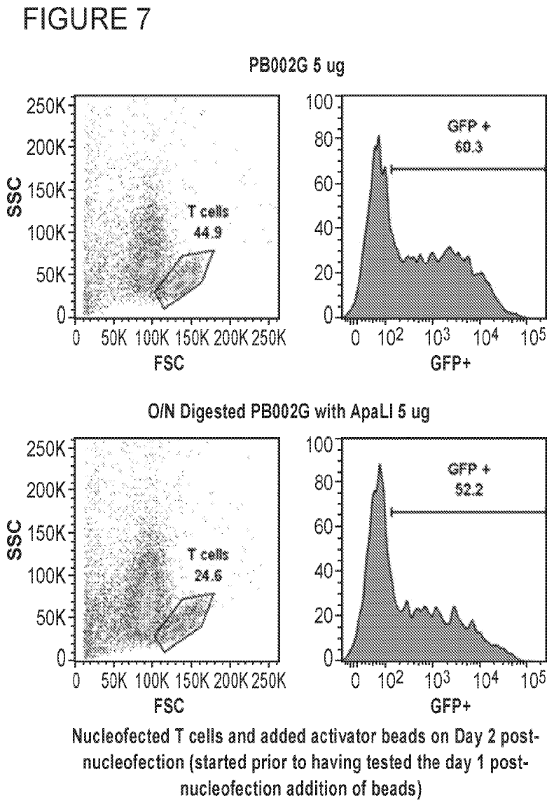

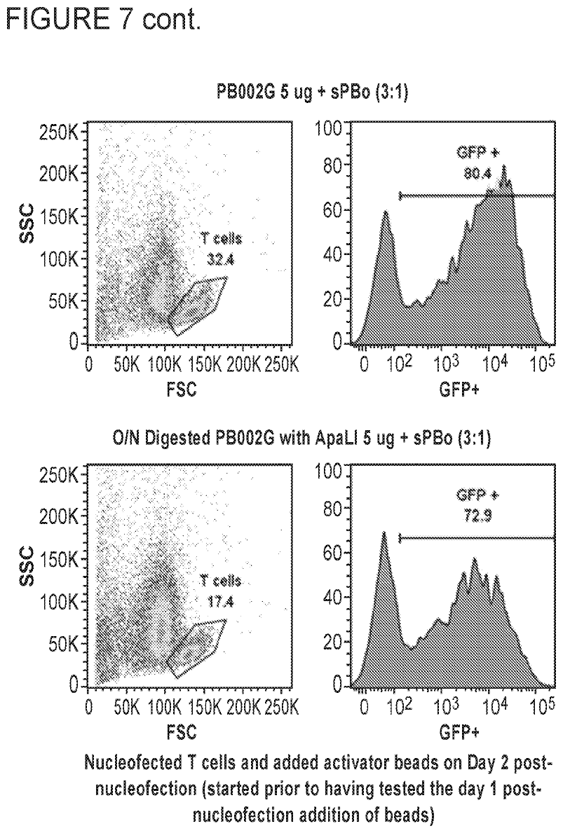

We performed a pilot experiment to see whether or not plasmid transposon needed to be circular, or if it could be delivered to the cell in a linear fashion. To test this, transposon was incubated overnight with a restriction enzyme (ApaLI) to linearize the plasmid. Either uncut or linearized plasmid is electroporated into primary T lymphocytes. GFP expression was assessed 2 days later. FIG. 7 shows that linearized plasmid was also efficiently delivered to the cell nucleus. These data demonstrate that linear transposon products can also be efficiently electroporated into primary human T cells.

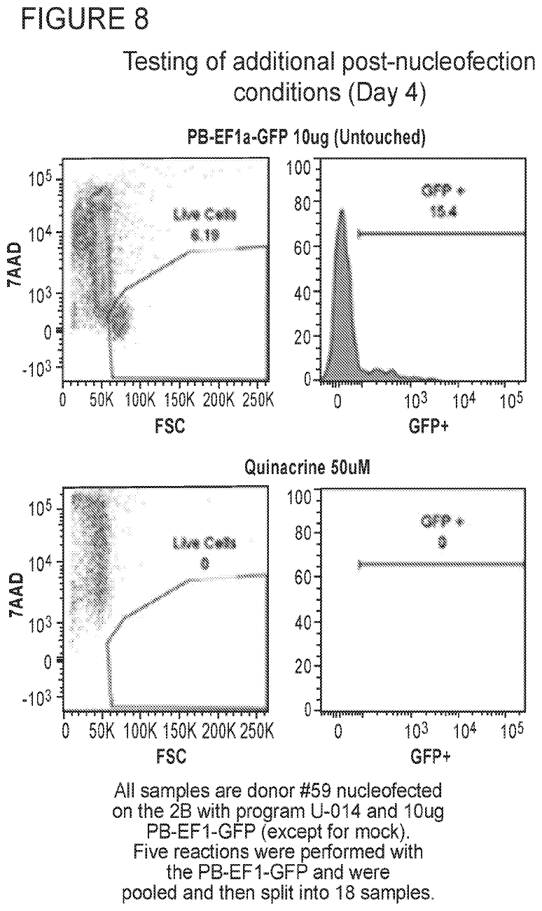

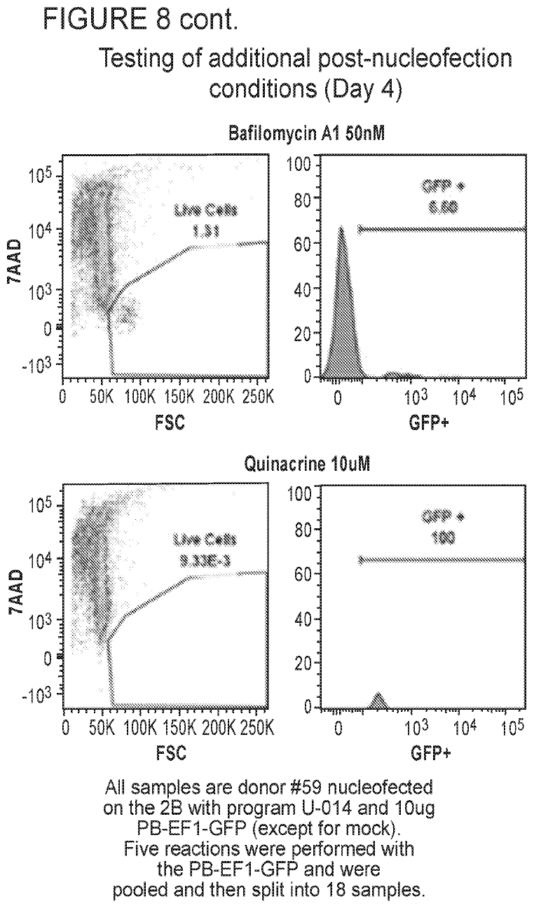

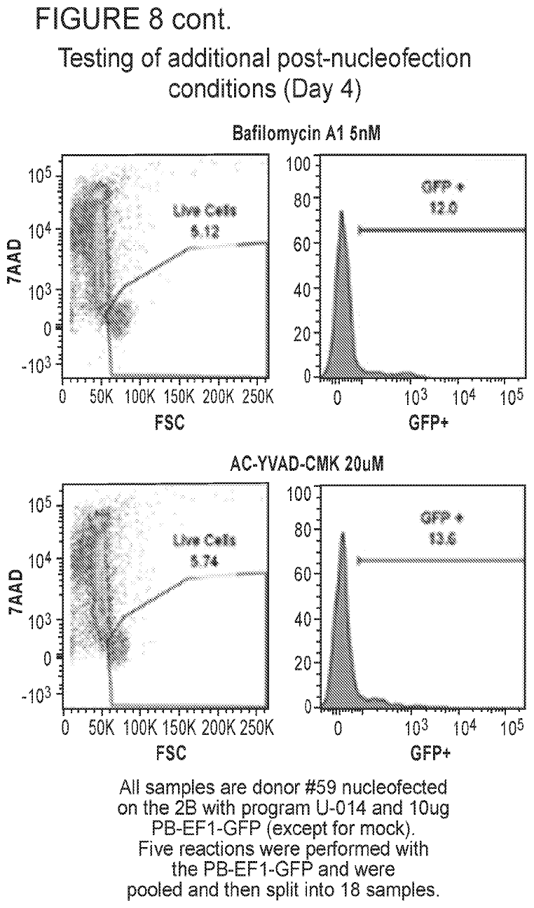

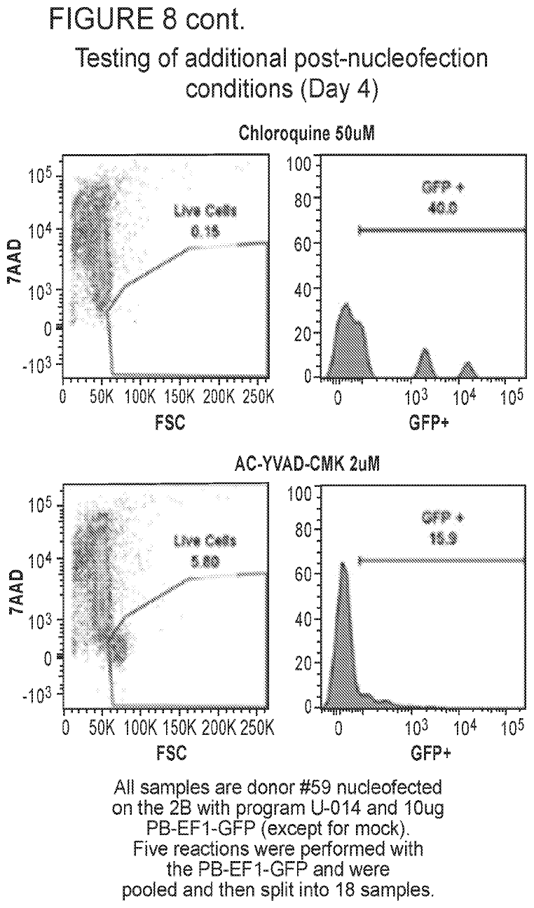

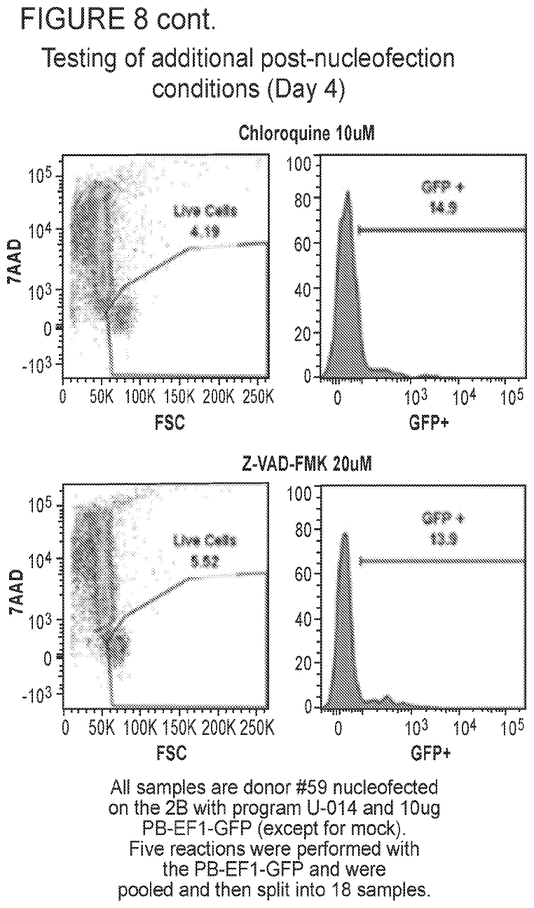

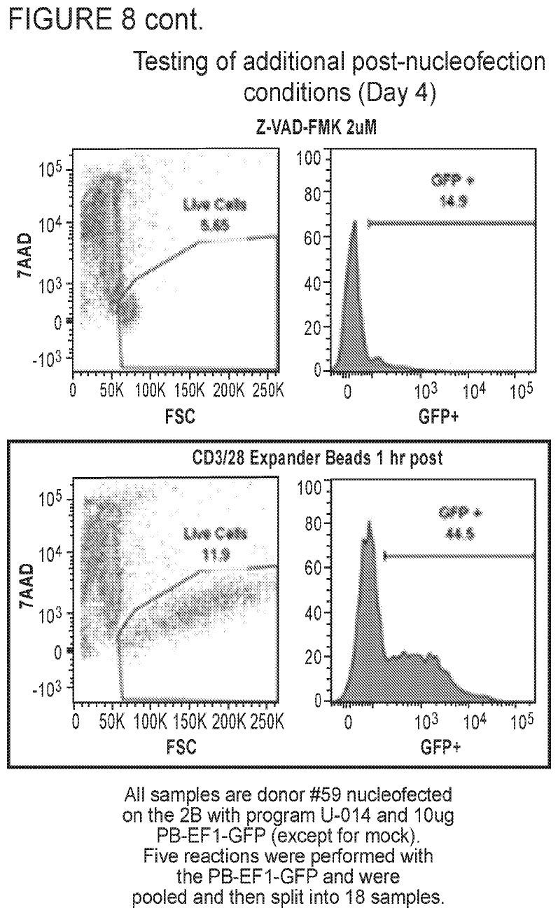

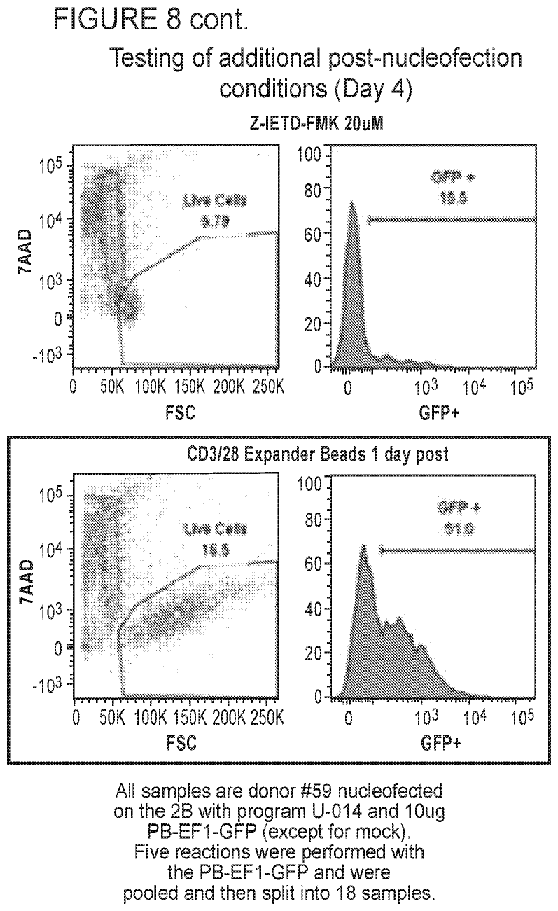

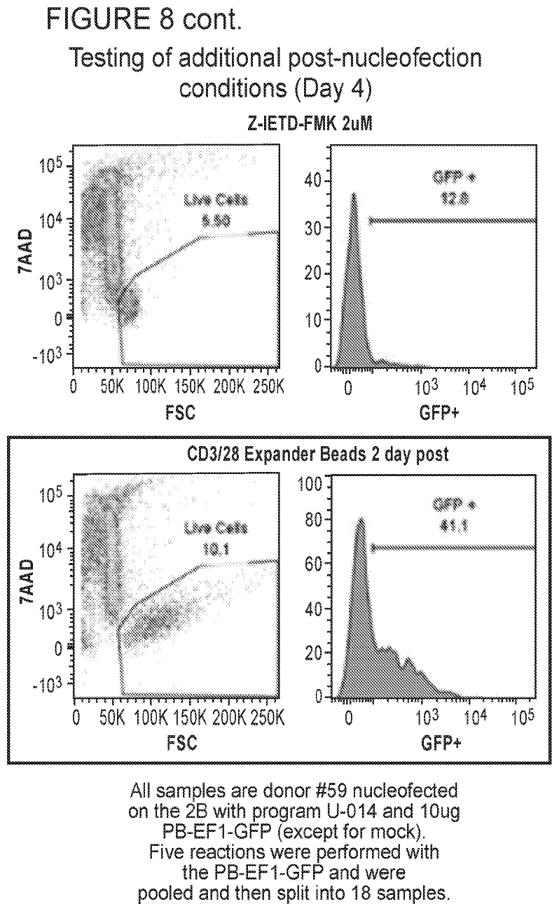

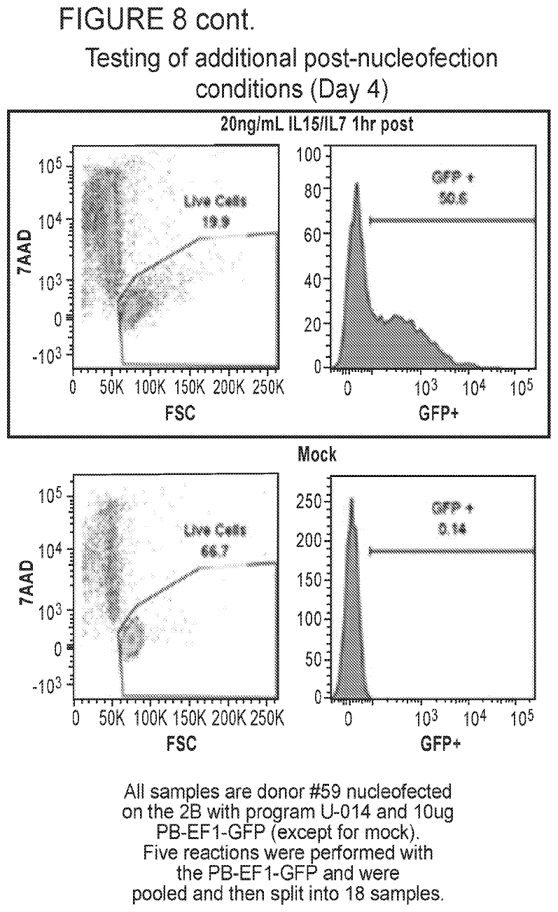

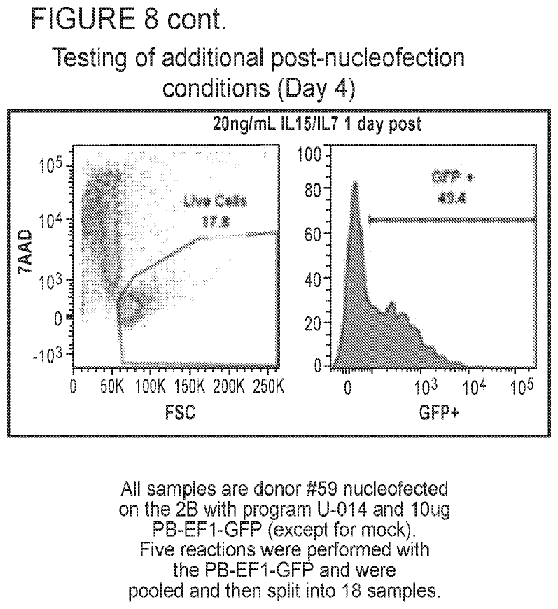

We show above that plasmid DNA is toxic in primary T lymphocytes, but we have observed that this toxic effect is not as dramatic in tumor cell lines and other transformed cells. Based upon this observation, we hypothesized that primary T lymphocytes may be refractory to plasmid DNA transfection due to heightened DNA sensing pathways, which would protect immune cells from infection by viruses and bacteria. If these data are a result of heightened DNA sensing mechanisms, then it may be possible to enhance plasmid transfection efficiency and/or cell viability by the addition of DNA sensing pathway inhibitors to the post-nucleofection reaction. Thus, we tested a number of different reagents that inhibited the TLR-9 pathway, caspase pathway, or those involved in cytoplasmic double stranded DNA sensing. These reagents include Bafilomycin Al, which is an autophagy inhibitor that interferes with endosomal acidification and blocks NFkB signaling by TLR9, Chloroquine, which is a TLR9 antagonist, Quinacrine, which is a TLR9 antagonist and a cGAS antagonist, AC-YVAD-CMK, which is a caspase 1 inhibitor targeting the AIM2 pathway, Z-VAD-FMK, which is a pan caspase inhibitor, Z-IETD-FMK, which is a caspase 8 inhibitor triggered by the TLR9 pathway. In addition, we also tested the stimulation of electroporated T cells by the addition of the cytokines IL7 and IL15, as well as the addition of anti-CD3 anti-CD28 Dynabeads® Human T-Expander CD3/CD28 beads. Results are displayed in FIG. 8. We found that few of the compounds or caspase inhibitors had any positive effect on cell viability at day 4 post-nucleofection at the doses tested. However, we acknowledge that further dosing studies may be required to better test these reagents. It may also be more effective to inhibit these pathways genetically. Two post-nucleofection conditions did enhance viability of the T cells. The addition of IL7 and IL15, whether they were added either 1 hour or 1 day following electroporation, enhanced viability over 3-fold when compared with introduction of the plasmid transposon alone without additional treatment. Furthermore, stimulation of the T cells post-nucleofection using either activator or expander beads also dramatically enhanced T cell viability; stimulation was better when the beads were added 1 hour or 1 day post-nucleofection as compared to adding the beads 2 days post. Lastly, we also tested ROCK inhibitor and the removal of dead cells from the culture using the Dead Cell Removal kit from Miltenyi, but saw no improvement in cell viability.

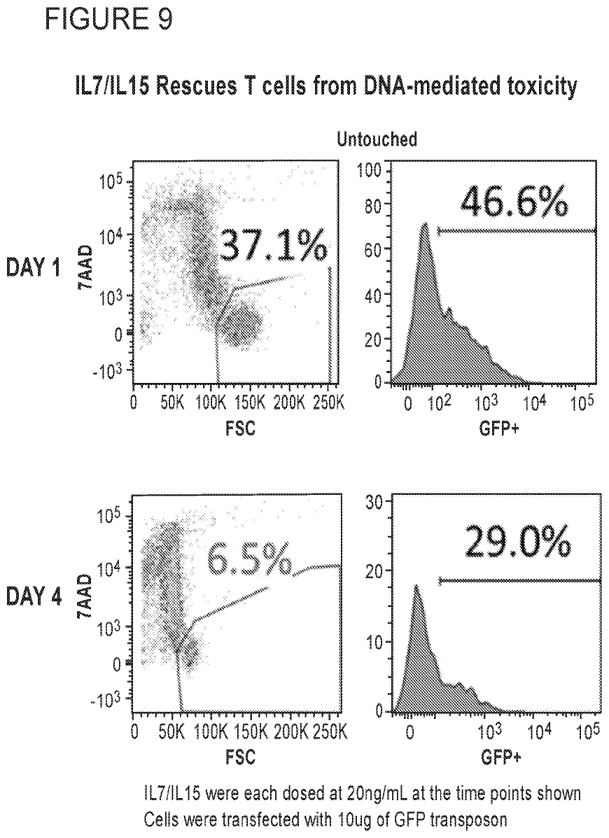

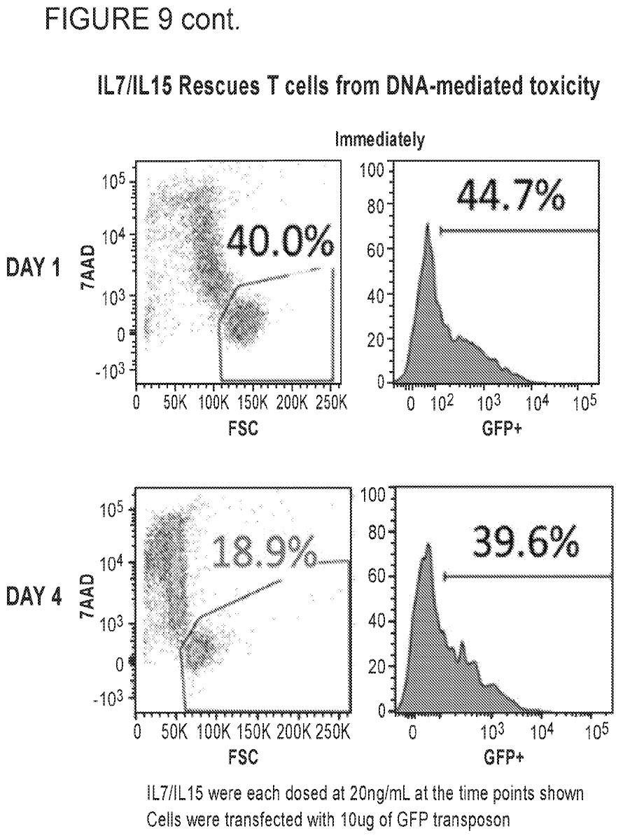

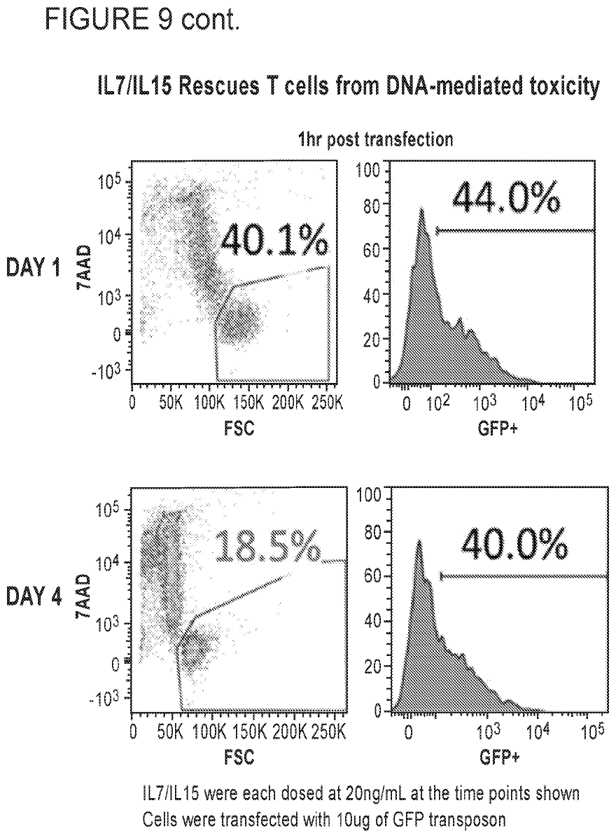

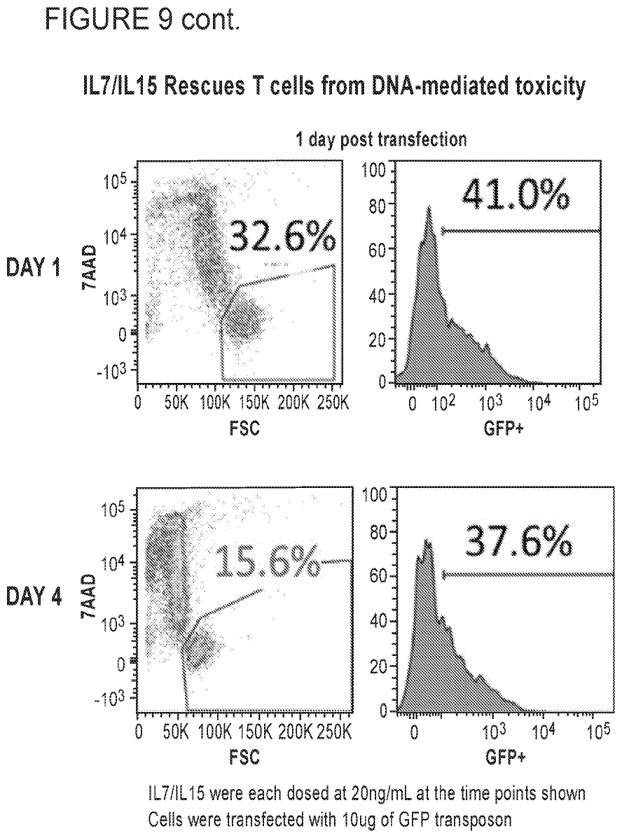

To further expand upon these findings demonstrating that stimulation of the T cells post-nucleofection improves viability, we repeated the study using the addition of the cytokine IL7 and IL15. FIG. 9 shows that the addition of these cytokines each at a dose of 20 ng/mL either immediately following nucleofection or up to 1 hour post enhanced cell viability up to 2.9-fold when compared to no treatment. Addition of these cytokines up to 1 day post-nucleofection also enhanced viability, but not as strong as the prior time points.

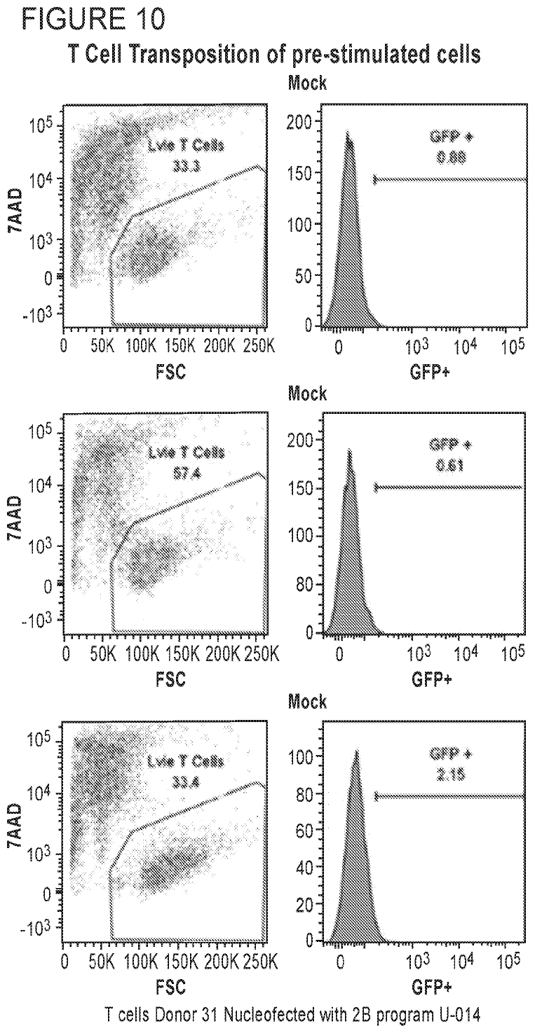

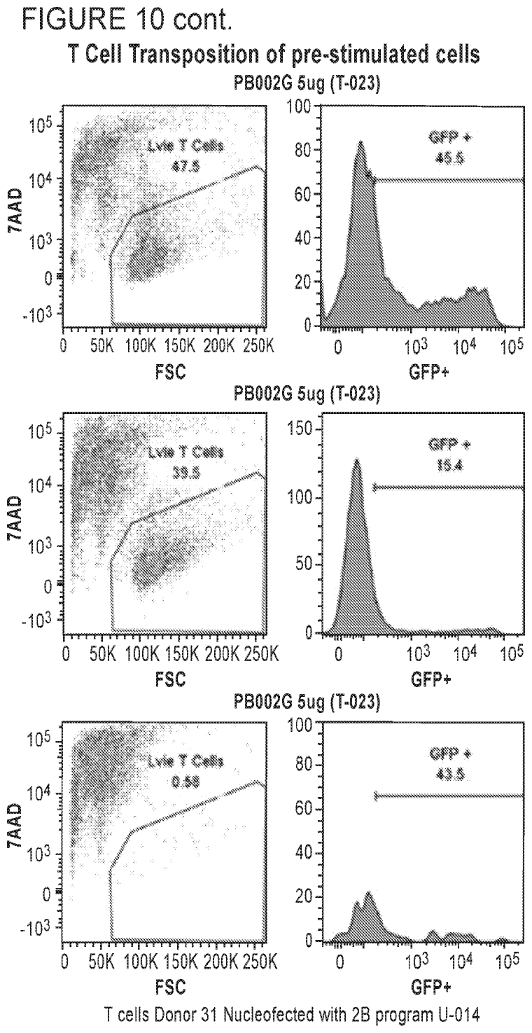

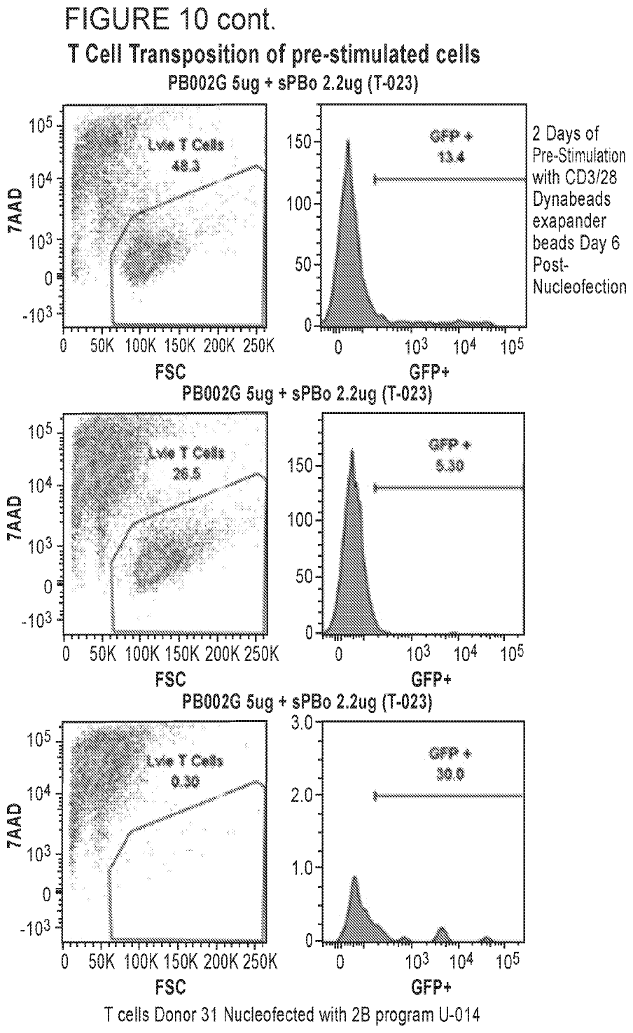

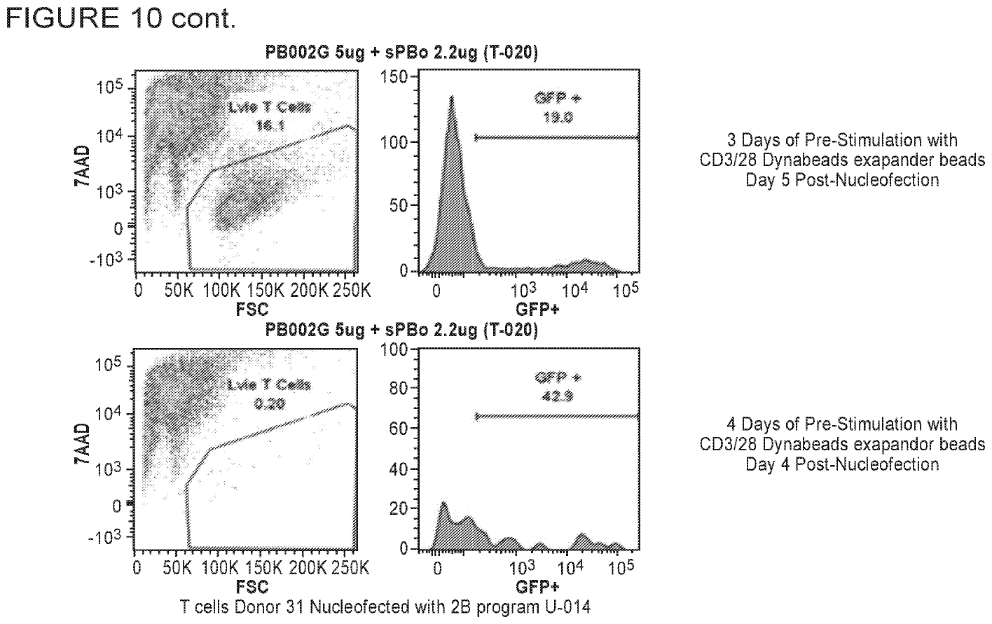

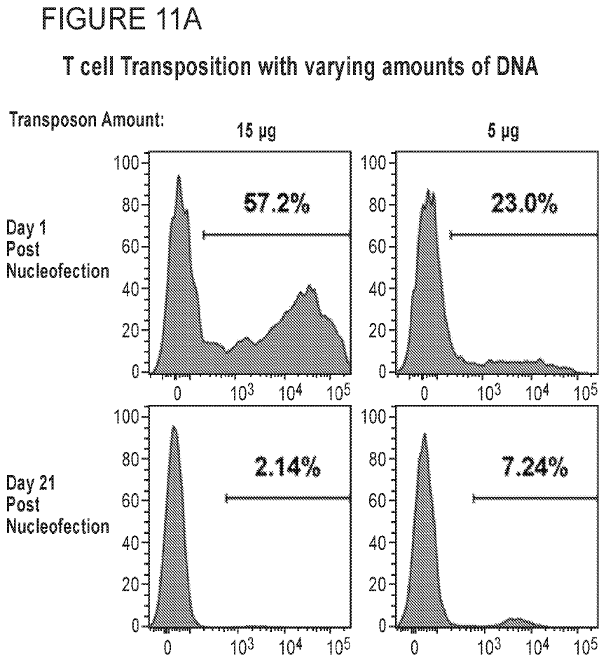

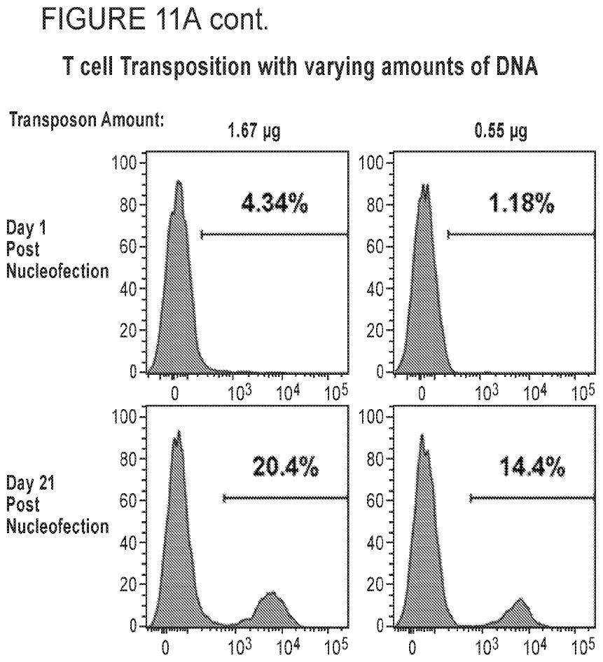

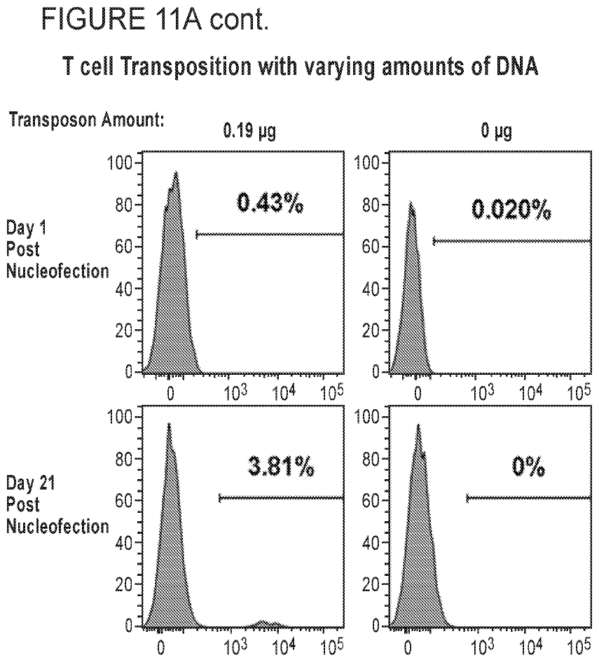

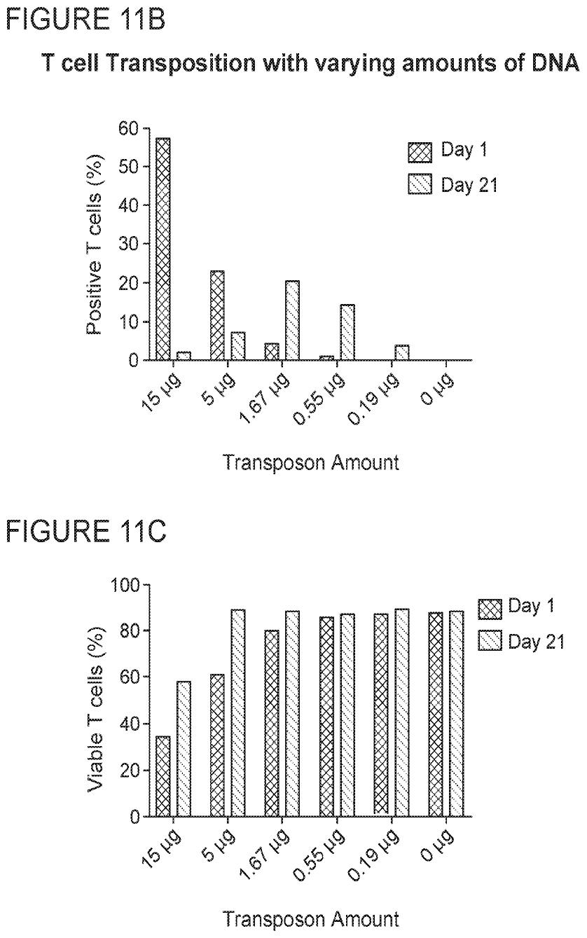

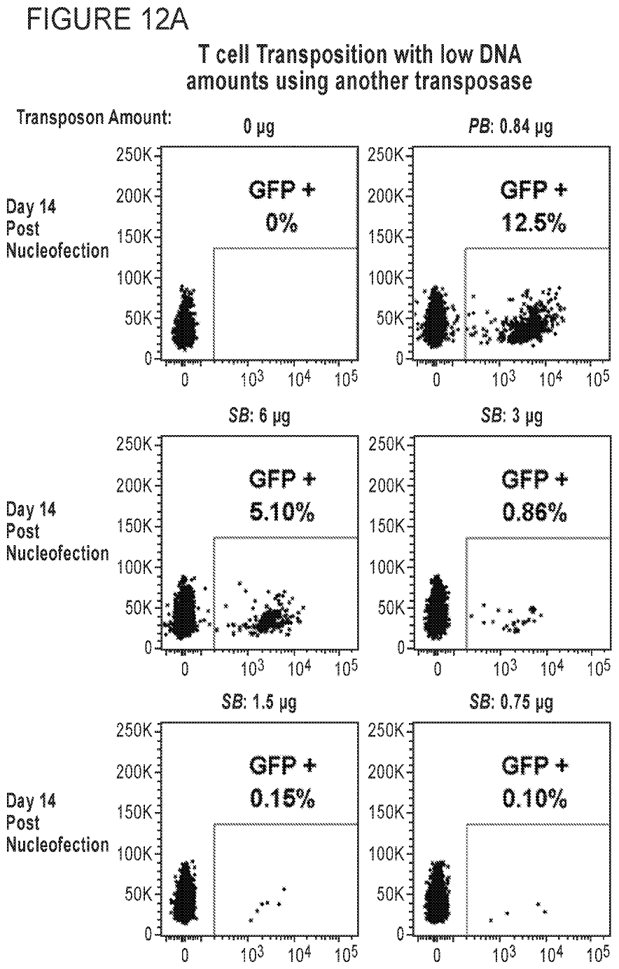

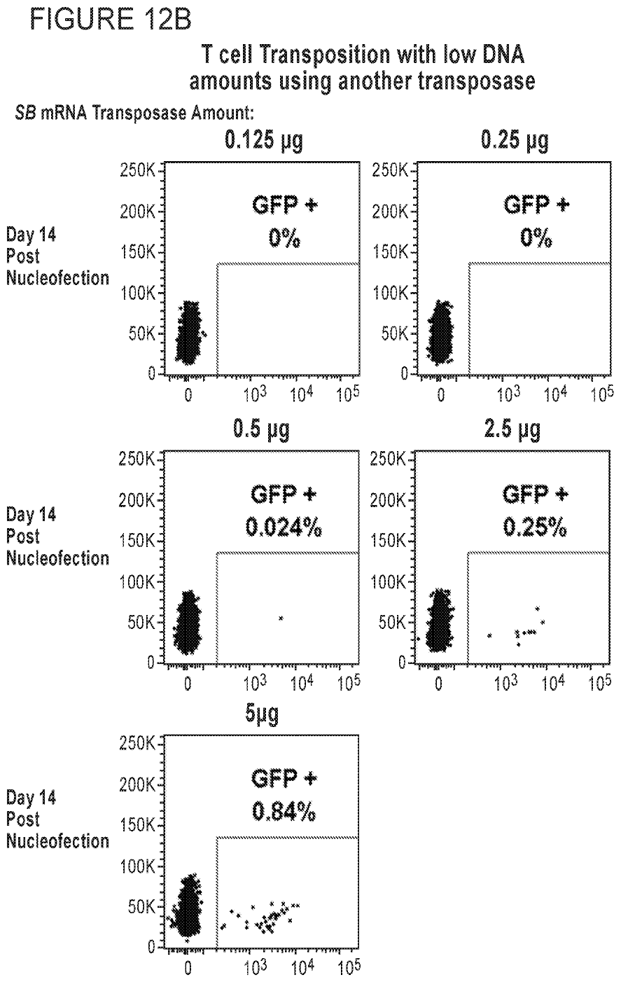

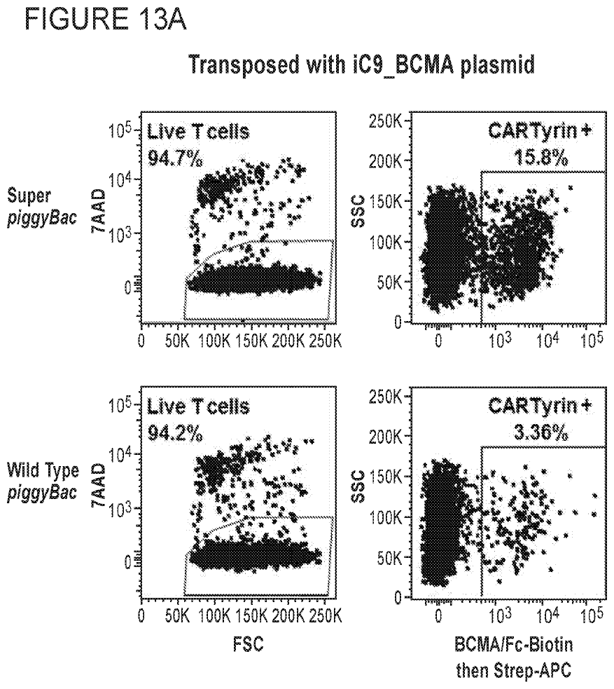

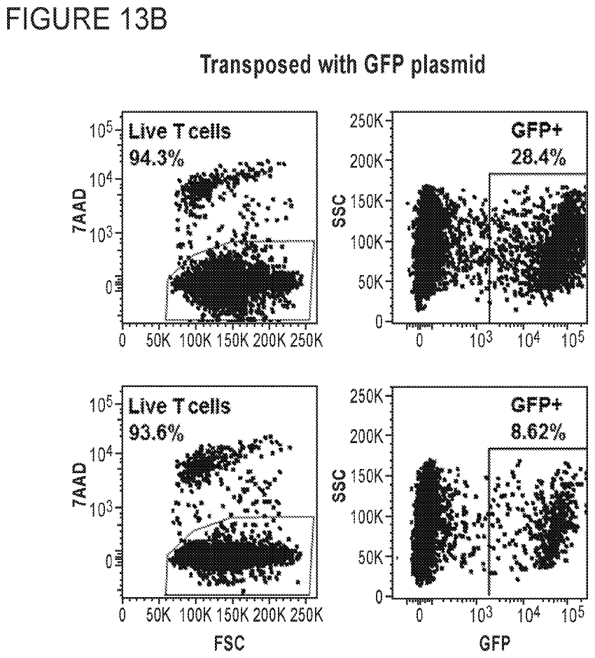

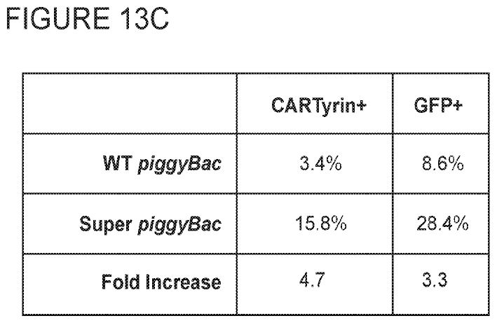

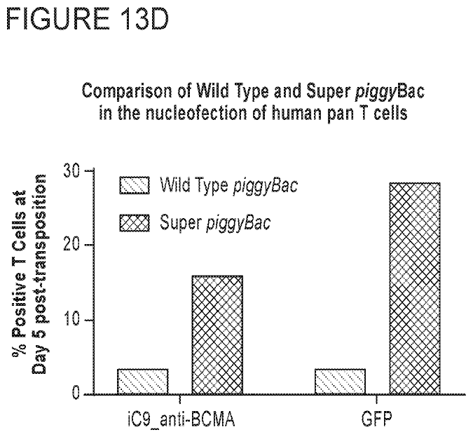

Since we found that immediate stimulation of the T cells post-nucleofection was able to increase cell viability, we hypothesized that stimulating the cells prior to nucleofection may also enhance viability and transfection efficiency. To test this, we stimulated primary T lymphocytes either 2, 3, or 4 days prior to transposon nucleofection. FIG. 10 shows that some level of transposition occurs when the transposon and the transposase are co-delivered after the T cells have been stimulated prior to the nucleofection reaction. The efficacy of pre-stimulation may be influenced by the kinetics of stimulation and may therefore be dependent upon the precise type of expander technology chosen.

### Example 2: Ex Vivo Genetic Modification of NK Cells

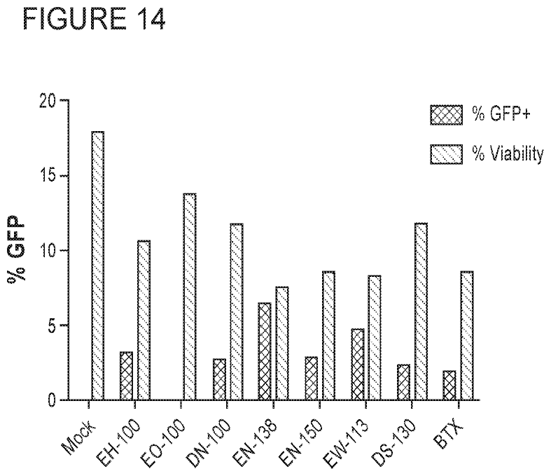

The piggyBac™ (PB) transposon system was used for genetically modifying human NK cells. Non-activated NK cells derived from CD3-depleted leukopheresis (containing CD14/CD19/CD56+ cells) were were electroporated with plasmid piggyBac transposon DNA encoding GFP and mRNA encoding Super piggyBac transposase using the program indicated in FIG. 14 from Lonza 4D nucleofector or BTX ECM 830 (500V, 700 usec pulse length, 0.2 mm electrode gap, one pulse). Transposed cells were co-cultured (stimulated) at day 2 with artificial antigen presenting cells (aAPCs). Fluorescent activated cell sorting (FACS) analysis of GFP percent at day 7 post-EP (day 5 post-stimulation) is shown in FIG. 14. Percent viability is the percentage of 7-Aminoactinomycin (7AAD)-negative cells at day 2 post-EP.

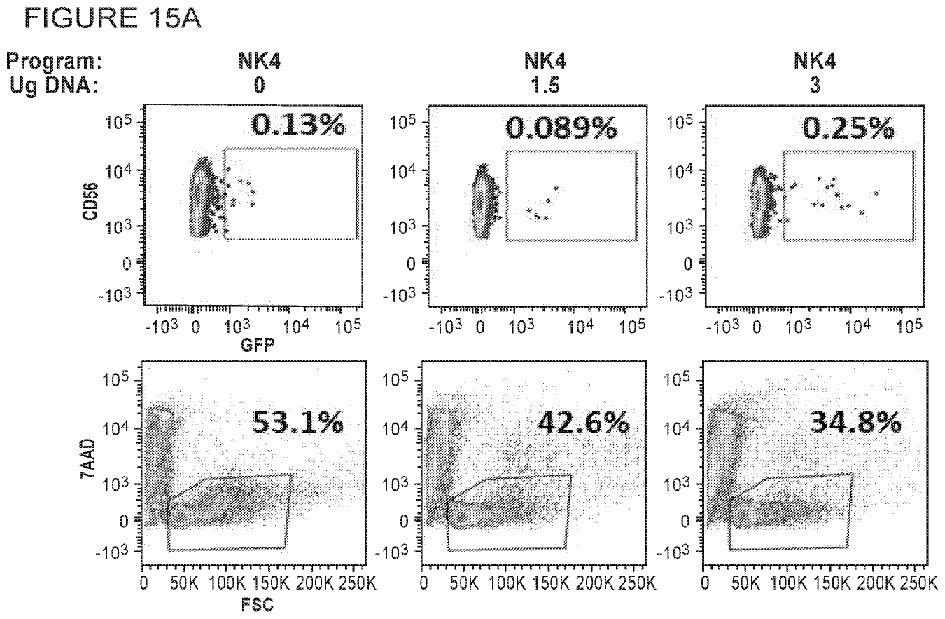

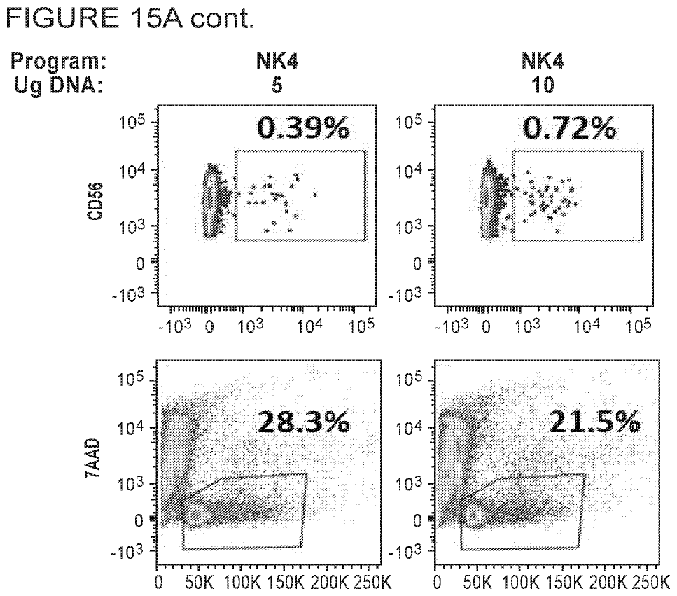

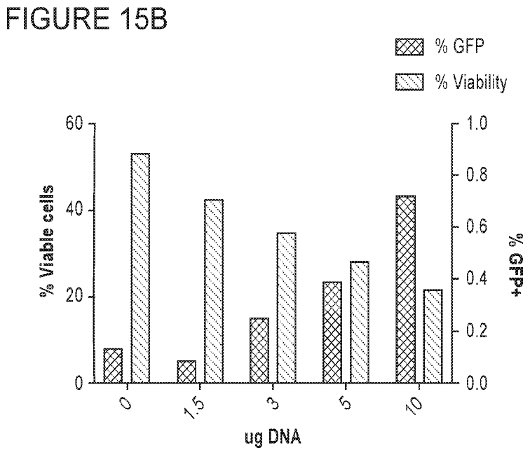

Transposition of non-activated NK cells from CD3-depleted leukopheresis (containing CD14/CD19/CD56+ cells) is shown in FIG. 15. Cells were electroporated with a plasmid piggyBac transposon encoding GFP and 5 ug mRNA encoding Super piggyBac transposase using the indicated Maxcyte electroporator program. Transposed cells were stimulated at day 2 with artificial antigen presenting cells (aAPCs). FACS plots (FIG. 15A) and a bar graph (FIG. 15B) from the analysis of percent GFP+ of CD56+ cells at day 6 post-EP and day 4 post-stimulation are shown. Percent viability is the percentage of 7AAD-negative cells at day 2 post EP.

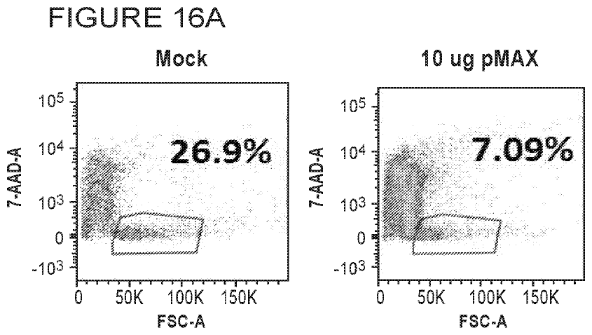

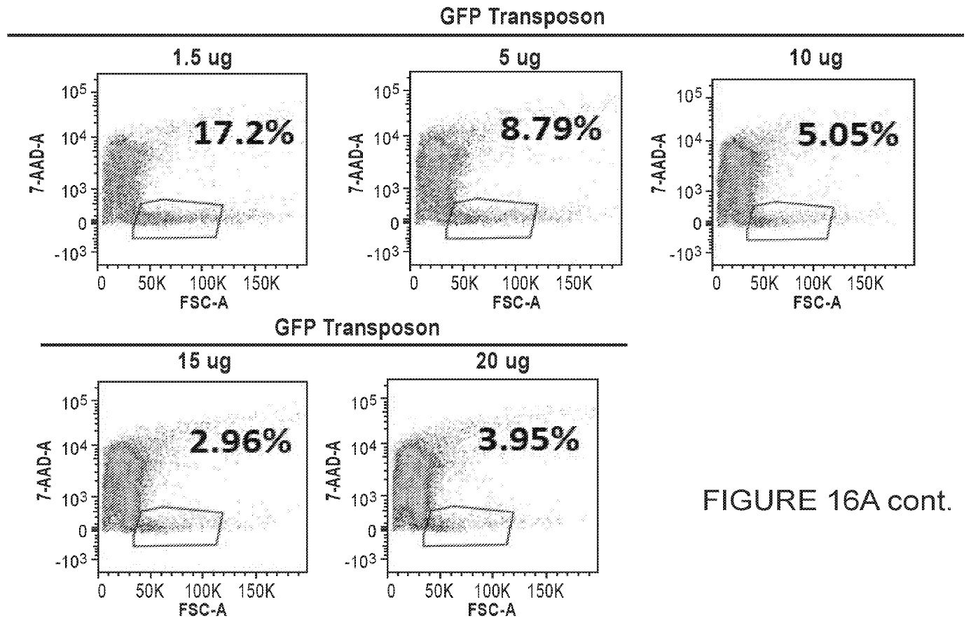

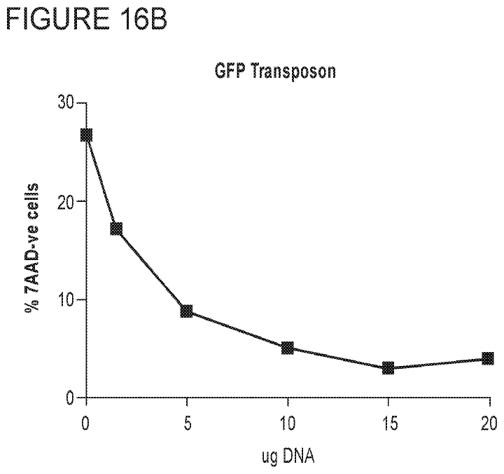

FIG. 16 shows that there is dose-dependent DNA-mediated cytotoxicity in NK cells. FACS analysis of live cells (7AAD-ve/FSC, or Forward Scatter) at day 2 post-EP using Lonza 4D Nucleofector program DN-100. FACS plots (FIG. 16A) are quantified in graph (FIG. 16B). 5x10E6 cells were electroporated per electroporation in 100 uL P3 buffer in cuvettes. Cells were electroporated with no DNA (Mock) or varying amounts of piggyBac GFP transposon co-delivered with 5 ug super piggyBac mRNA.

### Example 3: In Vitro Differentiation of piggyBac Modified HSPCs into B Cells

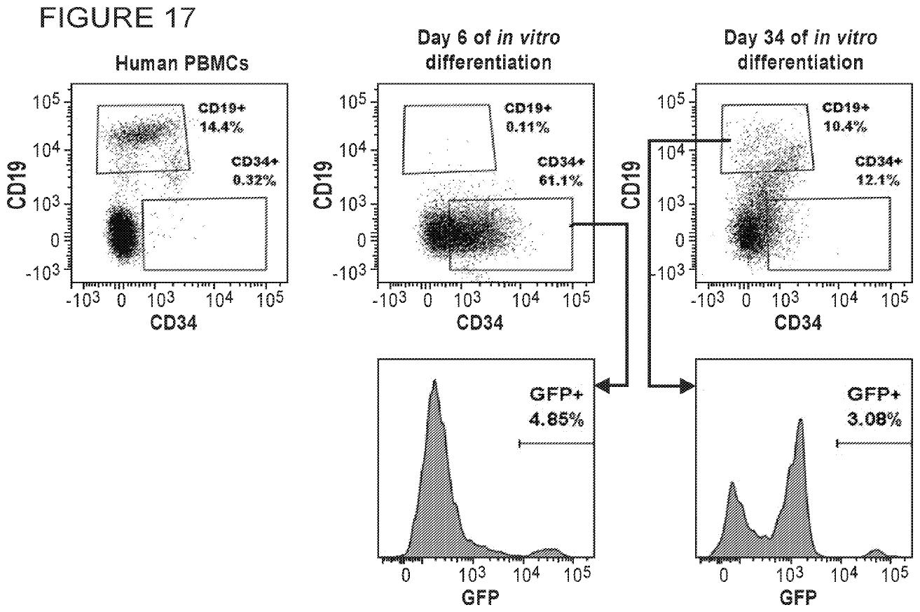

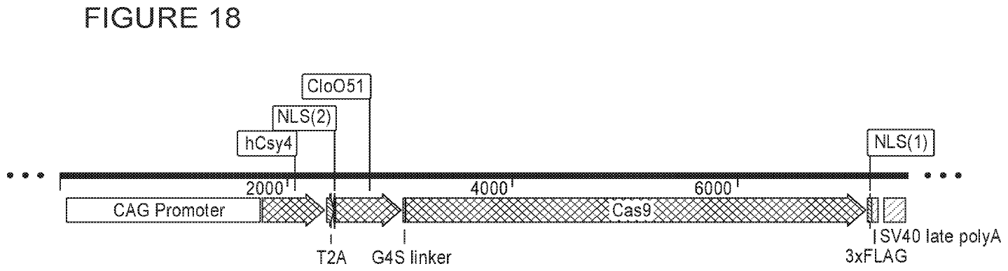

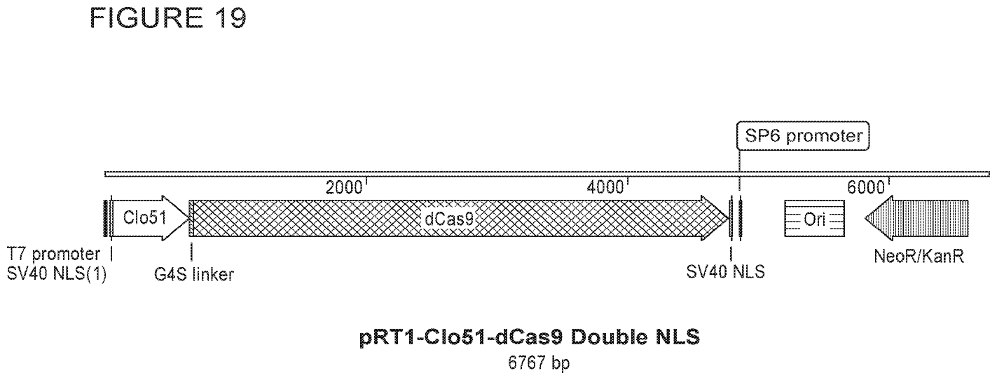

Human CD34+ HSPCs were electroporated with mRNA encoding Super piggyBac along with a piggyBac transposon encoding GFP. After electroporation, HSPCs were primed for B cell differentiation in presence of human IL-3, Flt3L, TPO, SCF, and G-CSF for 5 days. On day 6, cells were transferred to a layer of MS-5 feeder cells and fed bi-weekly, along with transfer to a fresh layer of feeders once per week. On day 34 of the in vitro differentiation process, CD19+B cells were generated and detectable in the culture (FIG. 17). A fraction of the B cells were positive for the GFP piggyBac transgene (FIG. 17, lower right panel) demonstrating that the piggyBac DNA Modification System can be used to modify HSPCs, which can then be later differentiated into more differentiated immune cell types. This technique allows for the derivation of genetically-modified immune cells from hematopoietic progenitors.

1-147. (canceled)

148. A method for the ex-vivo genetic modification of a stem cell comprising delivering to the stem cell:
(a) a nucleic acid or amino acid sequence comprising a sequence encoding a transposase enzyme;
(b) a recombinant and non-naturally occurring DNA sequence comprising a DNA sequence encoding a transposon; and
(c) differentiating the stem cell into an immune cell.

149. The method of claim 148, wherein the stem cell is a hematopoietic stem cell (HSC).

150. The method of claim 148, wherein the stem cell comprises the cell-surface marker phenotype CD34+ and CD38−.

151. The method of claim 148, wherein the stem cell comprises the cell-surface marker phenotype CD34+, CD38−, and CD90+.

152. The method of claim 148, wherein the stem cell comprises the cell-surface marker phenotype CD34+, CD38−, CD90+, and CD45RA−.

153. The method of claim 148, wherein the stem cell comprises the cell-surface marker phenotype CD34+, CD38−, CD90+, CD45RA−, and CD49f+.

154. The method of claim 148, wherein the immune cell is a T-lymphocyte, a Natural Killer (NK) cell, a Cytokine-induced Killer (CIK) cell, a Natural Killer T (NKT) cell, or a B lymphocyte (B Cell).

155. The method of claim 148, wherein the differentiating comprises priming the stem cell with any combination of IL-3, Flt3L, TPO, SCF, or G-CSF.

156. The method of claim 155, wherein the stem cell is primed for at least 3 days.

157. The method of claim 156, wherein the primed stem cell is transferred to a layer of feeder cells and fed bi-weekly.

158. The method of claim 157, wherein the primed stem cell is cultured with the feeder cells for at least 7 days.

159. The method of claim 148, wherein the method further comprises the step of stimulating the stem cell with at least one cytokine.

160. The method of claim 159, wherein the at least one cytokine is IL-2, IL-21, IL-7 or IL-15, or a combination thereof.

161. The method of claim 148, wherein the sequence encoding a transposase enzyme is an mRNA sequence.

162. The method of claim 148, wherein the sequence encoding a transposase enzyme is a DNA sequence.

163. The method of claim 148, wherein the sequence encoding a transposase enzyme is an amino acid sequence.

164. The method of claim 148, wherein the transposon is a piggyBac transposon, piggyBac-like transposon, Sleeping Beauty transposon, Tol2 transposon or Helraiser transposon.

165. The method of claim 148, wherein the transposase is a piggyBac transposase, piggyBac-like transposase, hyperactive piggyBac transposase, Super piggyBac (SPB) transposase, Sleeping Beauty transposase, hyperactive Sleeping Beauty (SB100X) transposase, Tol2 transposase or helitron transposase.

166. The method of claim 148, further comprising administering the immune cells to a subject in need thereof.

167. The method of claim 166, wherein the subject has cancer.
